SEQUENCE LISTING (1149 sequences)

SEQ ID NO: 1 - protein, 271 aa, synthetic construct
  REGION 1..271: note = recombinant human IL-1alpha
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 2 - protein, 269 aa, synthetic construct
  REGION 1..269: note = recombinant human IL-1beta
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 3 - protein, 270 aa, synthetic construct
  REGION 1..270: note = recombinant mouse IL-1alpha
MAKVPDLFEDLKNCYSENEDYSSAIDHLSLNQKSFYDASYGSLHETCTDQFVSLRTSETSKMSNFTFKESRVTVSATSSNGKILKKRRLSFSETFTEDDLQSITHDLEETIQPRSAPYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTDFQIS

SEQ ID NO: 4 - protein, 269 aa, synthetic construct
  REGION 1..269: note = recombinant mouse IL-1beta
MATVPELNCEMPPFDSDENDLFFEVDGPQKMKGCFQTFDLGCPDESIQLQISQQHINKSFRQAVSLIVAVEKLWQLPVSFPWTFQDEDMSTFFSFIFEEEPILCDSWDDDDNLLVCDVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 5 - protein, 177 aa, synthetic construct
  REGION 1..177: note = recombinant human IL-1 receptor antagonist
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 6 - protein, 178 aa, synthetic construct
  REGION 1..178: note = recombinant mouse IL-1 receptor antagonist
MEICWGPYSHLISLLLILLFHSEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 7 - DNA, 20 bp, Streptococcus pyogenes
  source 1..20: mol_type = other DNA
gtatcagcaacgtcaagcaa

SEQ ID NO: 8 - DNA, 20 bp, Streptococcus pyogenes
  source 1..20: mol_type = other DNA
ctgcaggtcatcttcagtga

SEQ ID NO: 9 - DNA, 20 bp, Streptococcus pyogenes
  source 1..20: mol_type = other DNA
tatcagcaacgtcaagcaac

SEQ ID NO: 10 - DNA, 20 bp, Streptococcus pyogenes
  source 1..20: mol_type = other DNA
gccatagcttgcatcataga

SEQ ID NO: 11 - DNA, 20 bp, Streptococcus pyogenes
  source 1..20: mol_type = other DNA
catcaacaagagcttcaggc

SEQ ID NO: 12 - DNA, 20 bp, Streptococcus pyogenes
  source 1..20: mol_type = other DNA
tgctctcatcaggacagccc

SEQ ID NO: 13 - DNA, 20 bp, Streptococcus pyogenes
  source 1..20: mol_type = other DNA
gctcatgtcctcatcctgga

SEQ ID NO: 14 - DNA, 20 bp, Streptococcus pyogenes
  source 1..20: mol_type = other DNA
cctcatcctggaaggtccac

SEQ ID NO: 15 - DNA, 21 bp, Staphylococcus aureus
  source 1..21: mol_type = other DNA
ttactccttaccttccagatc

SEQ ID NO: 16 - DNA, 21 bp, Staphylococcus aureus
  source 1..21: mol_type = other DNA
gaaactcagccgtctcttctt

SEQ ID NO: 17 - DNA, 21 bp, Staphylococcus aureus
  source 1..21: mol_type = other DNA
caacttcaccttcaaggagag

SEQ ID NO: 18 - DNA, 21 bp, Staphylococcus aureus
  source 1..21: mol_type = other DNA
gtgtctttcccgtggaccttc

SEQ ID NO: 19 - DNA, 21 bp, Staphylococcus aureus
  source 1..21: mol_type = other DNA
cacagcttctccacagccaca

SEQ ID NO: 20 - DNA, 21 bp, Staphylococcus aureus
  source 1..21: mol_type = other DNA
gtgctgctgcgagatttgaag

SEQ ID NO: 21 - RNA, 20 nt, Streptococcus pyogenes
  source 1..20: mol_type = other RNA
gtatcagcaacgtcaagcaa

SEQ ID NO: 22 - RNA, 20 nt, Streptococcus pyogenes
  source 1..20: mol_type = other RNA
ctgcaggtcatcttcagtga

SEQ ID NO: 23 - RNA, 20 nt, Streptococcus pyogenes
  source 1..20: mol_type = other RNA
tatcagcaacgtcaagcaac

SEQ ID NO: 24 - RNA, 20 nt, Streptococcus pyogenes
  source 1..20: mol_type = other RNA
gccatagcttgcatcataga

SEQ ID NO: 25 - RNA, 20 nt, Streptococcus pyogenes
  source 1..20: mol_type = other RNA
catcaacaagagcttcaggc

SEQ ID NO: 26 - RNA, 20 nt, Streptococcus pyogenes
  source 1..20: mol_type = other RNA
tgctctcatcaggacagccc

SEQ ID NO: 27 - RNA, 20 nt, Streptococcus pyogenes
  source 1..20: mol_type = other RNA
gctcatgtcctcatcctgga

SEQ ID NO: 28 - RNA, 20 nt, Streptococcus pyogenes
  source 1..20: mol_type = other RNA
cctcatcctggaaggtccac

SEQ ID NO: 29 - RNA, 21 nt, Staphylococcus aureus
  source 1..21: mol_type = other RNA
ttactccttaccttccagatc

SEQ ID NO: 30 - RNA, 21 nt, Staphylococcus aureus
  source 1..21: mol_type = other RNA
gaaactcagccgtctcttctt

SEQ ID NO: 31 - RNA, 21 nt, Staphylococcus aureus
  source 1..21: mol_type = other RNA
caacttcaccttcaaggagag

SEQ ID NO: 32 - RNA, 21 nt, Staphylococcus aureus
  source 1..21: mol_type = other RNA
gtgtctttcccgtggaccttc

SEQ ID NO: 33 - RNA, 21 nt, Staphylococcus aureus
  source 1..21: mol_type = other RNA
cacagcttctccacagccaca

SEQ ID NO: 34 - RNA, 21 nt, Staphylococcus aureus
  source 1..21: mol_type = other RNA
gtgctgctgcgagatttgaag

SEQ ID NO: 35 - RNA, 81 nt, synthetic construct
  misc_feature 1..81: note = Sau Cas9
  source 1..81: mol_type = other RNA
gttatagtactctggaaacagaatctactaaaacaaggcaaaatgccgtgtttatctcgtcaacttgttggcgagattttt

SEQ ID NO: 36 - RNA, 82 nt, synthetic construct
  misc_feature 1..82: note = Spy Cas9
  source 1..82: mol_type = other RNA
gttatagagctagaaatagcaagttaaaataaggctagtccgttatcaacttgaaaaagtggcaccgagtcggtgctttttt

SEQ ID NO: 37 - DNA, 20 bp, Homo sapiens
  source 1..20: mol_type = other DNA
gccatagcttacatgataga

SEQ ID NO: 38 - DNA, 20 bp, Homo sapiens
  source 1..20: mol_type = other DNA
tccttctatcatgtaagcta

SEQ ID NO: 39 - DNA, 20 bp, Homo sapiens
  source 1..20: mol_type = other DNA
ccatgcagccttcatggagt

SEQ ID NO: 40 - DNA, 20 bp, Homo sapiens
  source 1..20: mol_type = other DNA
tccatgcagccttcatggag

SEQ ID NO: 41 - DNA, 20 bp, Homo sapiens
  source 1..20: mol_type = other DNA
agctatggcccactccatga

SEQ ID NO: 42 - DNA, 20 bp, Homo sapiens
  source 1..20: mol_type = other DNA
attgatccatgcagccttca

SEQ ID NO: 43 - DNA, 20 bp, Homo sapiens
  source 1..20: mol_type = other DNA
cccactccatgaaggctgca

SEQ ID NO: 44 - DNA, 20 bp, Homo sapiens
  source 1..20: mol_type = other DNA
gctctccttgaaggtaagct

SEQ ID NO: 45 - DNA, 20 bp, Homo sapiens
  source 1..20: mol_type = other DNA
taccaccatgctctccttga

SEQ ID NO: 46 - DNA, 20 bp, Homo sapiens
  source 1..20: mol_type = other DNA
gcttaccttcaaggagagca

SEQ ID NO: 47 - DNA, 20 bp, Homo sapiens
  source 1..20: mol_type = other DNA
taccttcaaggagagcatgg

SEQ ID NO: 48 - DNA, 20 bp, Homo sapiens
  source 1..20: mol_type = other DNA
atggtggtagtagcaaccaa

SEQ ID NO: 49 - DNA, 20 bp, Homo sapiens
  source 1..20: mol_type = other DNA
tggtggtagtagcaaccaac

SEQ ID NO: 50 - DNA, 20 bp, Homo sapiens
  source 1..20: mol_type = other DNA
cttcttcagaaccttcccgt

SEQ ID NO: 51 - DNA, 20 bp, Homo sapiens
  source 1..20: mol_type = other DNA
ggtagtagcaaccaacggga

SEQ ID NO: 52 - DNA, 20 bp, Homo sapiens
  source 1..20: mol_type = other DNA
ggaaggttctgaagaagaga

SEQ ID NO: 53 - DNA, 20 bp, Homo sapiens
  source 1..20: mol_type = other DNA
ctccaggtcatcatcagtga

SEQ ID NO: 54 - DNA, 20 bp, Homo sapiens
  source 1..20: mol_type = other DNA
catcactgatgatgacctgg

SEQ ID NO: 55 - DNA, 20 bp, Homo sapiens
  source 1..20: mol_type = other DNA
agtcattggcgatggcctcc

SEQ ID NO: 56 - DNA, 20 bp, Homo sapiens
  source 1..20: mol_type = other DNA
ttcctctgagtcattggcga

SEQ ID NO: 57 - DNA, 20 bp, Homo sapiens
  source 1..20: mol_type = other DNA
tcccatgtgtcgaagaagat

SEQ ID NO: 58 - DNA, 20 bp, Homo sapiens
  source 1..20: mol_type = other DNA
aacctatcttcttcgacaca

SEQ ID NO: 59 - DNA, 20 bp, Homo sapiens
  source 1..20: mol_type = other DNA
acctatcttcttcgacacat

SEQ ID NO: 60 - DNA, 20 bp, Homo sapiens
  source 1..20: mol_type = other DNA
cttcgacacatgggataacg

SEQ ID NO: 61 - DNA, 20 bp, Homo sapiens
  source 1..20: mol_type = other DNA
gtgcagttcagtgatcgtac

SEQ ID NO: 62 - DNA, 20 bp, Homo sapiens
  source 1..20: mol_type = other DNA
gatcactgaactgcacgctc

SEQ ID NO: 63 - DNA, 20 bp, Homo sapiens
  source 1..20: mol_type = other DNA
atcactgaactgcacgctcc

SEQ ID NO: 64 - DNA, 20 bp, Homo sapiens
  source 1..20: mol_type = other DNA
caaaaaagcttggtgatgtc

SEQ ID NO: 65 - DNA, 20 bp, Homo sapiens
  source 1..20: mol_type = other DNA
ccatatcctgtccctggagg

SEQ ID NO: 66 - DNA, 20 bp, Homo sapiens
  source 1..20: mol_type = other DNA
ctgaaagctctccacctcca

SEQ ID NO: 67 - DNA, 20 bp, Homo sapiens
  source 1..20: mol_type = other DNA
gctccatatcctgtccctgg

SEQ ID NO: 68 - DNA, 20 bp, Homo sapiens
  source 1..20: mol_type = other DNA
agctctccacctccagggac

SEQ ID NO: 69 - DNA, 20 bp, Homo sapiens
  source 1..20: mol_type = other DNA
gttgctccatatcctgtccc

SEQ ID NO: 70 - DNA, 20 bp, Homo sapiens
  source 1..20: mol_type = other DNA
ggacaggatatggagcaaca

SEQ ID NO: 71 - DNA, 20 bp, Canis sp.
  source 1..20: mol_type = other DNA
gtcacagctcatatcataga

SEQ ID NO: 72 - DNA, 20 bp, Canis sp.
  source 1..20: mol_type = other DNA
acatgcagtcctcatgaagt

SEQ ID NO: 73 - DNA, 20 bp, Canis sp.
  source 1..20: mol_type = other DNA
gacatgcagtcctcatgaag

SEQ ID NO: 74 - DNA, 20 bp, Canis sp.
  source 1..20: mol_type = other DNA
gagctgtgacccacttcatg

SEQ ID NO: 75 - DNA, 20 bp, Canis sp.
  source 1..20: mol_type = other DNA
ggatgtctttgagatttcag

SEQ ID NO: 76 - DNA, 20 bp, Canis sp.
  source 1..20: mol_type = other DNA
attttccttgaaggtaagct

SEQ ID NO: 77 - DNA, 20 bp, Canis sp.
  source 1..20: mol_type = other DNA
gacatcccagcttaccttca

SEQ ID NO: 78 - DNA, 20 bp, Canis sp.
  source 1..20: mol_type = other DNA
cttcaaggaaaatgtggtag

SEQ ID NO: 79 - DNA, 20 bp, Canis sp.
  source 1..20: mol_type = other DNA
gtggtagtggtggcagccaa

SEQ ID NO: 80 - DNA, 20 bp, Canis sp.
  source 1..20: mol_type = other DNA
tggtagtggtggcagccaat

SEQ ID NO: 81 - DNA, 20 bp, Canis sp.
  source 1..20: mol_type = other DNA
cttctttagaatcttcccat

SEQ ID NO: 82 - DNA, 20 bp, Canis sp.
  source 1..20: mol_type = other DNA
ggaagattctaaagaagaga

SEQ ID NO: 83 - DNA, 20 bp, Canis sp.
  source 1..20: mol_type = other DNA
aatgtcttccaggtcatcat

SEQ ID NO: 84 - DNA, 20 bp, Canis sp.
  source 1..20: mol_type = other DNA
attcatcaccgatgatgacc

SEQ ID NO: 85 - DNA, 20 bp, Canis sp.
  source 1..20: mol_type = other DNA
attgccaatgacacagaaga

SEQ ID NO: 86 - DNA, 20 bp, Canis sp.
  source 1..20: mol_type = other DNA
cctcatctaccagagaactg

SEQ ID NO: 87 - DNA, 20 bp, Canis sp.
  source 1..20: mol_type = other DNA
ccacagttctctggtagatg

SEQ ID NO: 88 - DNA, 20 bp, Canis sp.
  source 1..20: mol_type = other DNA
cacagttctctggtagatga

SEQ ID NO: 89 - DNA, 20 bp, Canis sp.
  source 1..20: mol_type = other DNA
gctggtgggagacttgcaac

SEQ ID NO: 90 - DNA, 20 bp, Canis sp.
  source 1..20: mol_type = other DNA
actcttgttacagagctggt

SEQ ID NO: 91 - DNA, 20 bp, Canis sp.
  source 1..20: mol_type = other DNA
gactcttgttacagagctgg

SEQ ID NO: 92 - DNA, 20 bp, Canis sp.
  source 1..20: mol_type = other DNA
tcagactcttgttacagagc

SEQ ID NO: 93 - DNA, 20 bp, Canis sp.
  source 1..20: mol_type = other DNA
agctctgtaacaagagtctg

SEQ ID NO: 94 - DNA, 20 bp, Canis sp.
  source 1..20: mol_type = other DNA
cgtgtcagtcattgtagctt

SEQ ID NO: 95 - DNA, 20 bp, Canis sp.
  source 1..20: mol_type = other DNA
tcctggaggacctgtgggca

SEQ ID NO: 96 - DNA, 20 bp, Canis sp.
  source 1..20: mol_type = other DNA
gctgaagaagccctgcccac

SEQ ID NO: 97 - DNA, 20 bp, Canis sp.
  source 1..20: mol_type = other DNA
catcctcctggaggacctgt

SEQ ID NO: 98 - DNA, 20 bp, Canis sp.
  source 1..20: mol_type = other DNA
tcatcctcctggaggacctg

SEQ ID NO: 99 - DNA, 20 bp, Canis sp.
  source 1..20: mol_type = other DNA
gccctgcccacaggtcctcc

SEQ ID NO: 100 - DNA, 20 bp, Canis sp.
  source 1..20: mol_type = other DNA
ctgcccacaggtcctccagg

SEQ ID NO: 101 - DNA, 20 bp, Canis sp.
  source 1..20: mol_type = other DNA
tcttcaggtcatcctcctgg

SEQ ID NO: 102 - DNA, 20 bp, Canis sp.
  source 1..20: mol_type = other DNA
tgctcttcaggtcatcctcc

SEQ ID NO: 103 - DNA, 20 bp, Canis sp.
  source 1..20: mol_type = other DNA
tgtagcaaaagatgctcttc

SEQ ID NO: 104 - DNA, 20 bp, Canis sp.
  source 1..20: mol_type = other DNA
ttttgctacatctttgaaga

SEQ ID NO: 105 - DNA, 20 bp, Equus caballus
  source 1..20: mol_type = other DNA
gtcatagcttgcatcataga

SEQ ID NO: 106 - DNA, 20 bp, Equus caballus
  source 1..20: mol_type = other DNA
ccatgcagtcctcaggaagt

SEQ ID NO: 107 - DNA, 20 bp, Equus caballus
  source 1..20: mol_type = other DNA
tccatgcagtcctcaggaag

SEQ ID NO: 108 - DNA, 20 bp, Equus caballus
  source 1..20: mol_type = other DNA
aagctatgacccacttcctg

SEQ ID NO: 109 - DNA, 20 bp, Equus caballus
  source 1..20: mol_type = other DNA
aatgtatccatgcagtcctc

SEQ ID NO: 110 - DNA, 20 bp, Equus caballus
  source 1..20: mol_type = other DNA
cccacttcctgaggactgca

SEQ ID NO: 111 - DNA, 20 bp, Equus caballus
  source 1..20: mol_type = other DNA
ggatgtcttagaggtttcag

SEQ ID NO: 112 - DNA, 20 bp, Equus caballus
  source 1..20: mol_type = other DNA
gttcagcttggatgtcttag

SEQ ID NO: 113 - DNA, 20 bp, Equus caballus
  source 1..20: mol_type = other DNA
gctctccttgaagttcagct

SEQ ID NO: 114 - DNA, 20 bp, Equus caballus
  source 1..20: mol_type = other DNA
gacatccaagctgaacttca

SEQ ID NO: 115 - DNA, 20 bp, Equus caballus
  source 1..20: mol_type = other DNA
gctgaacttcaaggagagcg

SEQ ID NO: 116 - DNA, 20 bp, Equus caballus
  source 1..20: mol_type = other DNA
cttcaaggagagcgtggtgc

SEQ ID NO: 117 - DNA, 20 bp, Equus caballus
  source 1..20: mol_type = other DNA
caaggagagcgtggtgctgg

SEQ ID NO: 118 - DNA, 20 bp, Equus caballus
  source 1..20: mol_type = other DNA
gtggtgctggtggcagccaa

SEQ ID NO: 119 - DNA, 20 bp, Equus caballus
  source 1..20: mol_type = other DNA
tggtgctggtggcagccaac

SEQ ID NO: 120 - DNA, 20 bp, Equus caballus
  source 1..20: mol_type = other DNA
cttcttcagagtcttcccgt

SEQ ID NO: 121 - DNA, 20 bp, Equus caballus
  source 1..20: mol_type = other DNA
ggaagactctgaagaagaga

SEQ ID NO: 122 - DNA, 20 bp, Equus caballus
  source 1..20: mol_type = other DNA
aatggcttccaggtcatcat

SEQ ID NO: 123 - DNA, 20 bp, Equus caballus
  source 1..20: mol_type = other DNA
gttcatcaccaatgatgacc

SEQ ID NO: 124 - DNA, 20 bp, Equus caballus
  source 1..20: mol_type = other DNA
ttcttctggatcattggcaa

SEQ ID NO: 125 - DNA, 20 bp, Equus caballus
  source 1..20: mol_type = other DNA
ggtggtgggagatttgcaac

SEQ ID NO: 126 - DNA, 20 bp, Equus caballus
  source 1..20: mol_type = other DNA
agtcttgttgtagaggtggt

SEQ ID NO: 127 - DNA, 20 bp, Equus caballus
  source 1..20: mol_type = other DNA
aagtcttgttgtagaggtgg

SEQ ID NO: 128 - DNA, 20 bp, Equus caballus
  source 1..20: mol_type = other DNA
tgaaagtcttgttgtagagg

SEQ ID NO: 129 - DNA, 20 bp, Equus caballus
  source 1..20: mol_type = other DNA
gtttgaaagtcttgttgtag

SEQ ID NO: 130 - DNA, 20 bp, Equus caballus
  source 1..20: mol_type = other DNA
acatgccatgtcaatcattg

SEQ ID NO: 131 - DNA, 20 bp, Equus caballus
  source 1..20: mol_type = other DNA
catgtcaatcattgtggctg

SEQ ID NO: 132 - DNA, 20 bp, Mus musculus
  source 1..20: mol_type = other DNA
gccatagcttgcatcataga

SEQ ID NO: 133 - DNA, 20 bp, Mus musculus
  source 1..20: mol_type = other DNA
tccttctatgatgcaagcta

SEQ ID NO: 134 - DNA, 20 bp, Mus musculus
  source 1..20: mol_type = other DNA
ggacatctttgacgtttcag

SEQ ID NO: 135 - DNA, 20 bp, Mus musculus
  source 1..20: mol_type = other DNA
gatgtccaacttcaccttca

SEQ ID NO: 136 - DNA, 20 bp, Mus musculus
  source 1..20: mol_type = other DNA
tgtcacccggctctccttga

SEQ ID NO: 137 - DNA, 20 bp, Mus musculus
  source 1..20: mol_type = other DNA
cttcaccttcaaggagagcc

SEQ ID NO: 138 - DNA, 20 bp, Mus musculus
  source 1..20: mol_type = other DNA
acgttgctgatactgtcacc

SEQ ID NO: 139 - DNA, 20 bp, Mus musculus
  source 1..20: mol_type = other DNA
gtatcagcaacgtcaagcaa

SEQ ID NO: 140 - DNA, 20 bp, Mus musculus
  source 1..20: mol_type = other DNA
tatcagcaacgtcaagcaac

SEQ ID NO: 141 - DNA, 20 bp, Mus musculus
  source 1..20: mol_type = other DNA
ggaagattctgaagaagaga

SEQ ID NO: 142 - DNA, 20 bp, Mus musculus
  source 1..20: mol_type = other DNA
ctgcaggtcatcttcagtga

SEQ ID NO: 143 - DNA, 20 bp, Mus musculus
  source 1..20: mol_type = other DNA
accttccagatcatgggtta

SEQ ID NO: 144 - DNA, 20 bp, Mus musculus
  source 1..20: mol_type = other DNA
ctccttaccttccagatcat

SEQ ID NO: 145 - DNA, 20 bp, Mus musculus
  source 1..20: mol_type = other DNA
tccataacccatgatctgga

SEQ ID NO: 146 - DNA, 20 bp, Mus musculus
  source 1..20: mol_type = other DNA
aacccatgatctggaaggta

SEQ ID NO: 147 - DNA, 20 bp, Mus musculus
  source 1..20: mol_type = other DNA
gacagcccaggtcaaaggtt

SEQ ID NO: 148 - DNA, 20 bp, Mus musculus
  source 1..20: mol_type = other DNA
atcaggacagcccaggtcaa

SEQ ID NO: 149 - DNA, 20 bp, Mus musculus
  source 1..20: mol_type = other DNA
tgcttccaaacctttgacct

SEQ ID NO: 150 - DNA, 20 bp, Mus musculus
  source 1..20: mol_type = other DNA
tgctctcatcaggacagccc

SEQ ID NO: 151 - DNA, 20 bp, Mus musculus
  source 1..20: mol_type = other DNA
tgaagctggatgctctcatc

SEQ ID NO: 152 - DNA, 20 bp, Mus musculus
  source 1..20: mol_type = other DNA
gctgctgcgagatttgaagc

SEQ ID NO: 153 - DNA, 20 bp, Mus musculus
  source 1..20: mol_type = other DNA
catcaacaagagcttcaggc

SEQ ID NO: 154 - DNA, 20 bp, Mus musculus
  source 1..20: mol_type = other DNA
gcaggcagtatcactcattg

SEQ ID NO: 155 - DNA, 20 bp, Mus musculus
  source 1..20: mol_type = other DNA
agtatcactcattgtggctg

SEQ ID NO: 156 - DNA, 20 bp, Mus musculus
  source 1..20: mol_type = other DNA
ttgtggctgtggagaagctg

SEQ ID NO: 157 - DNA, 20 bp, Mus musculus
  source 1..20: mol_type = other DNA
aaggtccacgggaaagacac

SEQ ID NO: 158 - DNA, 20 bp, Mus musculus
  source 1..20: mol_type = other DNA
agctacctgtgtctttcccg

SEQ ID NO: 159 - DNA, 20 bp, Mus musculus
  source 1..20: mol_type = other DNA
cctcatcctggaaggtccac

SEQ ID NO: 160 - DNA, 20 bp, Mus musculus
  source 1..20: mol_type = other DNA
tcctcatcctggaaggtcca

SEQ ID NO: 161 - DNA, 20 bp, Mus musculus
  source 1..20: mol_type = other DNA
gctcatgtcctcatcctgga

SEQ ID NO: 162 - DNA, 20 bp, Mus musculus
  source 1..20: mol_type = other DNA
cccgtggaccttccaggatg

SEQ ID NO: 163 - DNA, 20 bp, Mus musculus
  source 1..20: mol_type = other DNA
aggtgctcatgtcctcatcc

SEQ ID NO: 164 - DNA, 20 bp, Mus musculus
  source 1..20: mol_type = other DNA
ttcaaagatgaaggaaaaga

SEQ ID NO: 165 - DNA, 20 bp, Mus musculus
  source 1..20: mol_type = other DNA
agtaccttcttcaaagatga

SEQ ID NO: 166 - skipped

SEQ ID NO: 167 - DNA, 20 bp, Mus musculus
  source 1..20: mol_type = other DNA
ttttccttcatctttgaaga

SEQ ID NO: 168 - RNA, 20 nt, Homo sapiens
  source 1..20: mol_type = other RNA
gccatagcttacatgataga

SEQ ID NO: 169 - RNA, 20 nt, Homo sapiens
  source 1..20: mol_type = other RNA
tccttctatcatgtaagcta

SEQ ID NO: 170 - RNA, 20 nt, Homo sapiens
  source 1..20: mol_type = other RNA
ccatgcagccttcatggagt

SEQ ID NO: 171 - RNA, 20 nt, Homo sapiens
  source 1..20: mol_type = other RNA
tccatgcagccttcatggag

SEQ ID NO: 172 - RNA, 20 nt, Homo sapiens
  source 1..20: mol_type = other RNA
agctatggcccactccatga

SEQ ID NO: 173 - RNA, 20 nt, Homo sapiens
  source 1..20: mol_type = other RNA
attgatccatgcagccttca

SEQ ID NO: 174 - RNA, 20 nt, Homo sapiens
  source 1..20: mol_type = other RNA
cccactccatgaaggctgca

SEQ ID NO: 175 - RNA, 20 nt, Homo sapiens
  source 1..20: mol_type = other RNA
gctctccttgaaggtaagct

SEQ ID NO: 176 - RNA, 20 nt, Homo sapiens
  source 1..20: mol_type = other RNA
taccaccatgctctccttga

SEQ ID NO: 177 - RNA, 20 nt, Homo sapiens
  source 1..20: mol_type = other RNA
gcttaccttcaaggagagca

SEQ ID NO: 178 - RNA, 20 nt, Homo sapiens
  source 1..20: mol_type = other RNA
taccttcaaggagagcatgg

SEQ ID NO: 179 - RNA, 20 nt, Homo sapiens
  source 1..20: mol_type = other RNA
atggtggtagtagcaaccaa

SEQ ID NO: 180 - RNA, 20 nt, Homo sapiens
  source 1..20: mol_type = other RNA
tggtggtagtagcaaccaac

SEQ ID NO: 181 - RNA, 20 nt, Homo sapiens
  source 1..20: mol_type = other RNA
cttcttcagaaccttcccgt

SEQ ID NO: 182 - RNA, 20 nt, Homo sapiens
  source 1..20: mol_type = other RNA
ggtagtagcaaccaacggga

SEQ ID NO: 183 - RNA, 20 nt, Homo sapiens
  source 1..20: mol_type = other RNA
ggaaggttctgaagaagaga

SEQ ID NO: 184 - RNA, 20 nt, Homo sapiens
  source 1..20: mol_type = other RNA
ctccaggtcatcatcagtga

SEQ ID NO: 185 - RNA, 20 nt, Homo sapiens
  source 1..20: mol_type = other RNA
catcactgatgatgacctgg

SEQ ID NO: 186 - RNA, 20 nt, Homo sapiens
  source 1..20: mol_type = other RNA
agtcattggcgatggcctcc

SEQ ID NO: 187 - RNA, 20 nt, Homo sapiens
  source 1..20: mol_type = other RNA
ttcctctgagtcattggcga

SEQ ID NO: 188 - RNA, 20 nt, Homo sapiens
  source 1..20: mol_type = other RNA
tcccatgtgtcgaagaagat

SEQ ID NO: 189 - RNA, 20 nt, Homo sapiens
  source 1..20: mol_type = other RNA
aacctatcttcttcgacaca

SEQ ID NO: 190 - RNA, 20 nt, Homo sapiens
  source 1..20: mol_type = other RNA
acctatcttcttcgacacat

SEQ ID NO: 191 - RNA, 20 nt, Homo sapiens
  source 1..20: mol_type = other RNA
cttcgacacatgggataacg

SEQ ID NO: 192 - RNA, 20 nt, Homo sapiens
  source 1..20: mol_type = other RNA
gtgcagttcagtgatcgtac

SEQ ID NO: 193 - RNA, 20 nt, Homo sapiens
  source 1..20: mol_type = other RNA
gatcactgaactgcacgctc

SEQ ID NO: 194 - RNA, 20 nt, Homo sapiens
  source 1..20: mol_type = other RNA
atcactgaactgcacgctcc

SEQ ID NO: 195 - RNA, 20 nt, Homo sapiens
  source 1..20: mol_type = other RNA
caaaaaagcttggtgatgtc

SEQ ID NO: 196 - RNA, 20 nt, Homo sapiens
  source 1..20: mol_type = other RNA
ccatatcctgtccctggagg

SEQ ID NO: 197 - RNA, 20 nt, Homo sapiens
  source 1..20: mol_type = other RNA
ctgaaagctctccacctcca

SEQ ID NO: 198 - RNA, 20 nt, Homo sapiens
  source 1..20: mol_type = other RNA
gctccatatcctgtccctgg

SEQ ID NO: 199 - RNA, 20 nt, Homo sapiens
  source 1..20: mol_type = other RNA
agctctccacctccagggac

SEQ ID NO: 200 - RNA, 20 nt, Homo sapiens
  source 1..20: mol_type = other RNA
gttgctccatatcctgtccc

SEQ ID NO: 201 - RNA, 20 nt, Homo sapiens
  source 1..20: mol_type = other RNA
ggacaggatatggagcaaca

SEQ ID NO: 202 - RNA, 20 nt, Canis sp.
  source 1..20: mol_type = other RNA
gtcacagctcatatcataga

SEQ ID NO: 203 - RNA, 20 nt, Canis sp.
  source 1..20: mol_type = other RNA
acatgcagtcctcatgaagt

SEQ ID NO: 204 - RNA, 20 nt, Canis sp.
  source 1..20: mol_type = other RNA
gacatgcagtcctcatgaag

SEQ ID NO: 205 - RNA, 20 nt, Canis sp.
  source 1..20: mol_type = other RNA
gagctgtgacccacttcatg

SEQ ID NO: 206 - RNA, 20 nt, Canis sp.
  source 1..20: mol_type = other RNA
ggatgtctttgagatttcag

SEQ ID NO: 207 - RNA, 20 nt, Canis sp.
  source 1..20: mol_type = other RNA
attttccttgaaggtaagct

SEQ ID NO: 208 - RNA, 20 nt, Canis sp.
  source 1..20: mol_type = other RNA
gacatcccagcttaccttca

SEQ ID NO: 209 - RNA, 20 nt, Canis sp.
  source 1..20: mol_type = other RNA
cttcaaggaaaatgtggtag

SEQ ID NO: 210 - RNA, 20 nt, Canis sp.
  source 1..20: mol_type = other RNA
gtggtagtggtggcagccaa

SEQ ID NO: 211 - RNA, 20 nt, Canis sp.
  source 1..20: mol_type = other RNA
tggtagtggtggcagccaat

SEQ ID NO: 212 - RNA, 20 nt, Canis sp.
  source 1..20: mol_type = other RNA
cttctttagaatcttcccat

SEQ ID NO: 213 - RNA, 20 nt, Canis sp.
  source 1..20: mol_type = other RNA
ggaagattctaaagaagaga

SEQ ID NO: 214 - RNA, 20 nt, Canis sp.
  source 1..20: mol_type = other RNA
aatgtcttccaggtcatcat

SEQ ID NO: 215 - RNA, 20 nt, Canis sp.
  source 1..20: mol_type = other RNA
attcatcaccgatgatgacc

SEQ ID NO: 216 - RNA, 21 nt, Canis sp.
  source 1..21: mol_type = other RNA
attgccaatgacacagaagsa

SEQ ID NO: 217 - RNA, 20 nt, Canis sp.
  source 1..20: mol_type = other RNA
cctcatctaccagagaactg

SEQ ID NO: 218 - RNA, 20 nt, Canis sp.
  source 1..20: mol_type = other RNA
ccacagttctctggtagatg

SEQ ID NO: 219 - RNA, 20 nt, Canis sp.
  source 1..20: mol_type = other RNA
cacagttctctggtagatga

SEQ ID NO: 220 - RNA, 20 nt, Canis sp.
  source 1..20: mol_type = other RNA
gctggtgggagacttgcaac

SEQ ID NO: 221 - RNA, 20 nt, Canis sp.
  source 1..20: mol_type = other RNA
actcttgttacagagctggt

SEQ ID NO: 222 - RNA, 20 nt, Canis sp.
  source 1..20: mol_type = other RNA
gactcttgttacagagctgg

SEQ ID NO: 223 - RNA, 20 nt, Canis sp.
  source 1..20: mol_type = other RNA
tcagactcttgttacagagc

SEQ ID NO: 224 - RNA, 20 nt, Canis sp.
  source 1..20: mol_type = other RNA
agctctgtaacaagagtctg

SEQ ID NO: 225 - RNA, 20 nt, Canis sp.
  source 1..20: mol_type = other RNA
cgtgtcagtcattgtagctt

SEQ ID NO: 226 - RNA, 20 nt, Canis sp.
  source 1..20: mol_type = other RNA
tcctggaggacctgtgggca

SEQ ID NO: 227 - RNA, 20 nt, Canis sp.
  source 1..20: mol_type = other RNA
gctgaagaagccctgcccac

SEQ ID NO: 228 - RNA, 20 nt, Canis sp.
  source 1..20: mol_type = other RNA
catcctcctggaggacctgt

SEQ ID NO: 229 - RNA, 20 nt, Canis sp.
  source 1..20: mol_type = other RNA
tcatcctcctggaggacctg

SEQ ID NO: 230 - RNA, 20 nt, Canis sp.
  source 1..20: mol_type = other RNA
gccctgcccacaggtcctcc

SEQ ID NO: 231 - RNA, 20 nt, Canis sp.
  source 1..20: mol_type = other RNA
ctgcccacaggtcctccagg

SEQ ID NO: 232 - RNA, 20 nt, Canis sp.
  source 1..20: mol_type = other RNA
tcttcaggtcatcctcctgg

SEQ ID NO: 233 - RNA, 20 nt, Canis sp.
  source 1..20: mol_type = other RNA
tgctcttcaggtcatcctcc

SEQ ID NO: 234 - RNA, 20 nt, Canis sp.
  source 1..20: mol_type = other RNA
tgtagcaaaagatgctcttc

SEQ ID NO: 235 - RNA, 20 nt, Canis sp.
  source 1..20: mol_type = other RNA
ttttgctacatctttgaaga

SEQ ID NO: 236 - RNA, 20 nt, Equus caballus
  source 1..20: mol_type = other RNA
gtcatagcttgcatcataga

SEQ ID NO: 237 - RNA, 20 nt, Equus caballus
  source 1..20: mol_type = other RNA
ccatgcagtcctcaggaagt

SEQ ID NO: 238 - RNA, 20 nt, Equus caballus
  source 1..20: mol_type = other RNA
tccatgcagtcctcaggaag

SEQ ID NO: 239 - RNA, 20 nt, Equus caballus
  source 1..20: mol_type = other RNA
aagctatgacccacttcctg

SEQ ID NO: 240 - RNA, 20 nt, Equus caballus
  source 1..20: mol_type = other RNA
aatgtatccatgcagtcctc

SEQ ID NO: 241 - RNA, 20 nt, Equus caballus
  source 1..20: mol_type = other RNA
cccacttcctgaggactgca

SEQ ID NO: 242 - RNA, 20 nt, Equus caballus
  source 1..20: mol_type = other RNA
ggatgtcttagaggtttcag

SEQ ID NO: 243 - RNA, 20 nt, Equus caballus
  source 1..20: mol_type = other RNA
gttcagcttggatgtcttag

SEQ ID NO: 244 - RNA, 20 nt, Equus caballus
  source 1..20: mol_type = other RNA
gctctccttgaagttcagct

SEQ ID NO: 245 - RNA, 20 nt, Equus caballus
  source 1..20: mol_type = other RNA
gacatccaagctgaacttca

SEQ ID NO: 246 - RNA, 20 nt, Equus caballus
  source 1..20: mol_type = other RNA
gctgaacttcaaggagagcg

SEQ ID NO: 247 - RNA, 20 nt, Equus caballus
  source 1..20: mol_type = other RNA
cttcaaggagagcgtggtgc

SEQ ID NO: 248 - RNA, 20 nt, Equus caballus
  source 1..20: mol_type = other RNA
caaggagagcgtggtgctgg

SEQ ID NO: 249 - RNA, 20 nt, Equus caballus
  source 1..20: mol_type = other RNA
gtggtgctggtggcagccaa

SEQ ID NO: 250 - RNA, 20 nt, Equus caballus
  source 1..20: mol_type = other RNA
tggtgctggtggcagccaac

SEQ ID NO: 251 - RNA, 20 nt, Equus caballus
  source 1..20: mol_type = other RNA
cttcttcagagtcttcccgt

SEQ ID NO: 252 - RNA, 20 nt, Equus caballus
  source 1..20: mol_type = other RNA
ggaagactctgaagaagaga

SEQ ID NO: 253 - RNA, 20 nt, Equus caballus
  source 1..20: mol_type = other RNA
aatggcttccaggtcatcat

SEQ ID NO: 254 - RNA, 20 nt, Equus caballus
  source 1..20: mol_type = other RNA
gttcatcaccaatgatgacc

SEQ ID NO: 255 - RNA, 20 nt, Equus caballus
  source 1..20: mol_type = other RNA
ttcttctggatcattggcaa

SEQ ID NO: 256 - RNA, 20 nt, Equus caballus
  source 1..20: mol_type = other RNA
ggtggtgggagatttgcaac

SEQ ID NO: 257 - RNA, 20 nt, Equus caballus
  source 1..20: mol_type = other RNA
agtcttgttgtagaggtggt

SEQ ID NO: 258 - RNA, 20 nt, Equus caballus
  source 1..20: mol_type = other RNA
aagtcttgttgtagaggtgg

SEQ ID NO: 259 - RNA, 20 nt, Equus caballus
  source 1..20: mol_type = other RNA
tgaaagtcttgttgtagagg

SEQ ID NO: 260 - RNA, 20 nt, Equus caballus
  source 1..20: mol_type = other RNA
gtttgaaagtcttgttgtag

SEQ ID NO: 261 - RNA, 20 nt, Equus caballus
  source 1..20: mol_type = other RNA
acatgccatgtcaatcattg

SEQ ID NO: 262 - RNA, 20 nt, Equus caballus
  source 1..20: mol_type = other RNA
catgtcaatcattgtggctg

SEQ ID NO: 263 - RNA, 20 nt, Mus musculus
  source 1..20: mol_type = other RNA
gccatagcttgcatcataga

SEQ ID NO: 264 - RNA, 20 nt, Mus musculus
  source 1..20: mol_type = other RNA
tccttctatgatgcaagcta

SEQ ID NO: 265 - RNA, 20 nt, Mus musculus
  source 1..20: mol_type = other RNA
ggacatctttgacgtttcag

SEQ ID NO: 266 - RNA, 20 nt, Mus musculus
  source 1..20: mol_type = other RNA
gatgtccaacttcaccttca

SEQ ID NO: 267 - RNA, 20 nt, Mus musculus
  source 1..20: mol_type = other RNA
tgtcacccggctctccttga

SEQ ID NO: 268 - RNA, 20 nt, Mus musculus
  source 1..20: mol_type = other RNA
cttcaccttcaaggagagcc

SEQ ID NO: 269 - RNA, 20 nt, Mus musculus
  source 1..20: mol_type = other RNA
acgttgctgatactgtcacc

SEQ ID NO: 270 - RNA, 20 nt, Mus musculus
  source 1..20: mol_type = other RNA
gtatcagcaacgtcaagcaa

SEQ ID NO: 271 - RNA, 20 nt, Mus musculus
  source 1..20: mol_type = other RNA
tatcagcaacgtcaagcaac

SEQ ID NO: 272 - RNA, 20 nt, Mus musculus
  source 1..20: mol_type = other RNA
ggaagattctgaagaagaga

SEQ ID NO: 273 - RNA, 20 nt, Mus musculus
  source 1..20: mol_type = other RNA
ctgcaggtcatcttcagtga

SEQ ID NO: 274 - RNA, 20 nt, Mus musculus
  source 1..20: mol_type = other RNA
accttccagatcatgggtta

SEQ ID NO: 275 - RNA, 20 nt, Mus musculus
  source 1..20: mol_type = other RNA
ctccttaccttccagatcat

SEQ ID NO: 276 - RNA, 20 nt, Mus musculus
  source 1..20: mol_type = other RNA
tccataacccatgatctgga

SEQ ID NO: 277 - RNA, 20 nt, Mus musculus
  source 1..20: mol_type = other RNA
aacccatgatctggaaggta

SEQ ID NO: 278 - RNA, 20 nt, Mus musculus
  source 1..20: mol_type = other RNA
gacagcccaggtcaaaggtt

SEQ ID NO: 279 - RNA, 20 nt, Mus musculus
  source 1..20: mol_type = other RNA
atcaggacagcccaggtcaa

SEQ ID NO: 280 - RNA, 20 nt, Mus musculus
  source 1..20: mol_type = other RNA
tgcttccaaacctttgacct

SEQ ID NO: 281 - RNA, 20 nt, Mus musculus
  source 1..20: mol_type = other RNA
tgctctcatcaggacagccc

SEQ ID NO: 282 - RNA, 20 nt, Mus musculus
  source 1..20: mol_type = other RNA
tgaagctggatgctctcatc

SEQ ID NO: 283 - RNA, 20 nt, Mus musculus
  source 1..20: mol_type = other RNA
gctgctgcgagatttgaagc

SEQ ID NO: 284 - RNA, 20 nt, Mus musculus
  source 1..20: mol_type = other RNA
catcaacaagagcttcaggc

SEQ ID NO: 285 - RNA, 20 nt, Mus musculus
  source 1..20: mol_type = other RNA
gcaggcagtatcactcattg

SEQ ID NO: 286 - RNA, 20 nt, Mus musculus
  source 1..20: mol_type = other RNA
agtatcactcattgtggctg

SEQ ID NO: 287 - RNA, 20 nt, Mus musculus
  source 1..20: mol_type = other RNA
ttgtggctgtggagaagctg

SEQ ID NO: 288 - RNA, 20 nt, Mus musculus
  source 1..20: mol_type = other RNA
aaggtccacgggaaagacac

SEQ ID NO: 289 - RNA, 20 nt, Mus musculus
  source 1..20: mol_type = other RNA
agctacctgtgtctttcccg

SEQ ID NO: 290 - RNA, 20 nt, Mus musculus
  source 1..20: mol_type = other RNA
cctcatcctggaaggtccac

SEQ ID NO: 291 - RNA, 20 nt, Mus musculus
  source 1..20: mol_type = other RNA
tcctcatcctggaaggtcca

SEQ ID NO: 292 - RNA, 20 nt, Mus musculus
  source 1..20: mol_type = other RNA
gctcatgtcctcatcctgga

SEQ ID NO: 293 - RNA, 20 nt, Mus musculus
  source 1..20: mol_type = other RNA
cccgtggaccttccaggatg

SEQ ID NO: 294 - RNA, 20 nt, Mus musculus
  source 1..20: mol_type = other RNA
aggtgctcatgtcctcatcc

SEQ ID NO: 295 - RNA, 20 nt, Mus musculus
  source 1..20: mol_type = other RNA
ttcaaagatgaaggaaaaga

SEQ ID NO: 296 - RNA, 20 nt, Mus musculus
  source 1..20: mol_type = other RNA
agtaccttcttcaaagatga

SEQ ID NO: 297 - RNA, 20 nt, Mus musculus
  source 1..20: mol_type = other RNA
ttttccttcatctttgaaga

SEQ ID NO: 298 - RNA, 20 nt, synthetic construct
  misc_feature 1..20: note = IL1A
  source 1..20: mol_type = other RNA
tcaccagtgaaatttgacat

SEQ ID NO: 299 - RNA, 20 nt, synthetic construct
  misc_feature 1..20: note = IL1A
  source 1..20: mol_type = other RNA
atggtggtagtagcaaccaa

SEQ ID NO: 300 - RNA, 20 nt, synthetic construct
  misc_feature 1..20: note = IL1A
  source 1..20: mol_type = other RNA
agccaatgatcagtacctca

SEQ ID NO: 301 - RNA, 20 nt, synthetic construct
  misc_feature 1..20: note = IL1A
  source 1..20: mol_type = other RNA
cagagacagatgatcaatgg

SEQ ID NO: 302 - RNA, 20 nt, synthetic construct
  misc_feature 1..20: note = IL1A
  source 1..20: mol_type = other RNA
tgagactccagacctacgcc

SEQ ID NO: 303 - RNA, 20 nt, synthetic construct
  misc_feature 1..20: note = IL1A
  source 1..20: mol_type = other RNA
ttcaccagtgaaatttgaca

SEQ ID NO: 304 - RNA, 20 nt, synthetic construct
  misc_feature 1..20: note = IL1A
  source 1..20: mol_type = other RNA
ttttagaaatcatcaagcct

SEQ ID NO: 305 - RNA, 20 nt, synthetic construct
  misc_feature 1..20: note = IL1A
  source 1..20: mol_type = other RNA
tatgtaatgcagcagccgtg

SEQ ID NO: 306 - RNA, 20 nt, synthetic construct
  misc_feature 1..20: note = IL1A
  source 1..20: mol_type = other RNA
tggtggtagtagcaaccaac

SEQ ID NO: 307 - RNA, 20 nt, synthetic construct
  misc_feature 1..20: note = IL1A
  source 1..20: mol_type = other RNA
ggccatcgccaatgactcag

SEQ ID NO: 308 - RNA, 20 nt, synthetic construct
  misc_feature 1..20: note = IL1A
  source 1..20: mol_type = other RNA
atgtgaaatacaactttatg

SEQ ID NO: 309 - RNA, 20 nt, synthetic construct
  misc_feature 1..20: note = IL1A
  source 1..20: mol_type = other RNA
gccatagcttacatgataga

SEQ ID NO: 310 - RNA, 20 nt, synthetic construct
  misc_feature 1..20: note = IL1A
  source 1..20: mol_type = other RNA
agctatggcccactccatga

SEQ ID NO: 311 - RNA, 20 nt, synthetic construct
  misc_feature 1..20: note = IL1A
  source 1..20: mol_type = other RNA
atctgaaagtcagtgataga

SEQ ID NO: 312 - RNA, 20 nt, synthetic construct
  misc_feature 1..20: note = IL1A
  source 1..20: mol_type = other RNA
agccgtgaggtactgatcat

SEQ ID NO: 313 - RNA, 20 nt, synthetic construct
  misc_feature 1..20: note = IL1A
  source 1..20: mol_type = other RNA
attcagagacagatgatcaa

SEQ ID NO: 314 - RNA, 20 nt, synthetic construct
  misc_feature 1..20: note = IL1A
  source 1..20: mol_type = other RNA
tgaaagtcagtgatagaggg

SEQ ID NO: 315 - RNA, 20 nt, synthetic construct
  misc_feature 1..20: note = IL1A
  source 1..20: mol_type = other RNA
tggcaataaacaagtttgga

SEQ ID NO: 316 - RNA, 20 nt, Synthetic construct
  source 1..20: mol_type = other RNA
gcacccatgtcaaatttcac

SEQ ID NO: 317 - RNA, 20 nt, Synthetic construct
  source 1..20: mol_type = other RNA
ttcctctgagtcattggcga

SEQ ID NO: 318 - RNA, 20 nt, Synthetic construct
  source 1..20: mol_type = other RNA
atcgccaatgactcagagga

SEQ ID NO: 319 - RNA, 20 nt, Synthetic construct
  source 1..20: mol_type = other RNA
ctcttcttctgggaaactca

SEQ ID NO: 320 - RNA, 20 nt, Synthetic construct
  source 1..20: mol_type = other RNA
aagctaaaaggtgctgacct

SEQ ID NO: 321 - RNA, 20 nt, Synthetic construct
  source 1..20: mol_type = other RNA
ctcgaattatactttgattg

SEQ ID NO: 322 - RNA, 20 nt, Synthetic construct
  source 1..20: mol_type = other RNA
tggaaaatcaggcgtaggtc

SEQ ID NO: 323 - RNA, 20 nt, Synthetic construct
  source 1..20: mol_type = other RNA
cttcttcagaaccttcccgt

SEQ ID NO: 324 - RNA, 20 nt, Synthetic construct
  source 1..20: mol_type = other RNA
gttggtctcactacctgtga

SEQ ID NO: 325 - RNA, 20 nt, Synthetic construct
  source 1..20: mol_type = other RNA
tccttctatcatgtaagcta

SEQ ID NO: 326 - RNA, 20 nt, Synthetic construct
  source 1..20: mol_type = other RNA
gatactggaaaaccaggcgt

SEQ ID NO: 327 - RNA, 20 nt, Synthetic construct
  source 1..20: mol_type = other RNA
taccttcaaggagagcatgg

SEQ ID NO: 328 - RNA, 20 nt, Synthetic construct
  source 1..20: mol_type = other RNA
agaccaaccagtgctgctga

SEQ ID NO: 329 - RNA, 20 nt, Synthetic construct
  source 1..20: mol_type = other RNA
tatctgaaagtcagtgatag

SEQ ID NO: 330 - RNA, 20 nt, Synthetic construct
  source 1..20: mol_type = other RNA
ggcaataaacaagtttggat

SEQ ID NO: 331 - RNA, 20 nt, Synthetic construct
  source 1..20: mol_type = other RNA
catcactgatgatgacctgg

SEQ ID NO: 332 - RNA, 20 nt, Synthetic construct
  source 1..20: mol_type = other RNA
gagatacccaaaaccatcac

SEQ ID NO: 333 - RNA, 20 nt, Synthetic construct
  source 1..20: mol_type = other RNA
ttttgagattcttagaatca

SEQ ID NO: 334 - RNA, 20 nt, Synthetic construct
  source 1..20: mol_type = other RNA
ttgccacaaagcaagactac

SEQ ID NO: 335 - RNA, 20 nt, Synthetic construct
  source 1..20: mol_type = other RNA
tgaccttcagcagcactggt

SEQ ID NO: 336 - RNA, 20 nt, Synthetic construct
  source 1..20: mol_type = other RNA
ctttcagatactggaaaacc

SEQ ID NO: 337 - RNA, 20 nt, Synthetic construct
  source 1..20: mol_type = other RNA
ccatgcagccttcatggagt

SEQ ID NO: 338 - RNA, 20 nt, Synthetic construct
  source 1..20: mol_type = other RNA
ggtaagcttggatgttttag

SEQ ID NO: 339 - RNA, 20 nt, Synthetic construct
  source 1..20: mol_type = other RNA
ttacataatctggatgaaga

SEQ ID NO: 340 - RNA, 20 nt, Synthetic construct
  source 1..20: mol_type = other RNA
ggctgctgcattacataatc

SEQ ID NO: 341 - RNA, 20 nt, Synthetic construct
  source 1..20: mol_type = other RNA
acattgctcaggaagctaaa

SEQ ID NO: 342 - RNA, 20 nt, Synthetic construct
  source 1..20: mol_type = other RNA
gcttaccttcaaggagagca

SEQ ID NO: 343 - RNA, 19 nt, Synthetic construct
  source 1..19: mol_type = other RNA
ttgtggcaataaacaagtt

SEQ ID NO: 344 - RNA, 20 nt, Synthetic construct
  source 1..20: mol_type = other RNA
ctccaggtcatcatcagtga

SEQ ID NO: 345 - RNA, 20 nt, Synthetic construct
  source 1..20: mol_type = other RNA
cacccagtagtcttgctttg

SEQ ID NO: 346 - RNA, 20 nt, Synthetic construct
  source 1..20: mol_type = other RNA
gagtttcccagaagaagagg

SEQ ID NO: 347 - RNA, 20 nt, Synthetic construct
  source 1..20: mol_type = other RNA
agttgtatttcacattgctc

SEQ ID NO: 348 - RNA, 20 nt, Synthetic construct
  source 1..20: mol_type = other RNA
ggtcatcatcagtgatggat

SEQ ID NO: 349 - RNA, 20 nt, Synthetic construct
  source 1..20: mol_type = other RNA
ttcaaacatgtctggaactt

SEQ ID NO: 350 - RNA, 20 nt, Synthetic construct
  source 1..20: mol_type = other RNA
ggtagtagcaaccaacggga

SEQ ID NO: 351 - RNA, 20 nt, Synthetic construct
  source 1..20: mol_type = other RNA
tccatgcagccttcatggag

SEQ ID NO: 352 - RNA, 20 nt, Synthetic construct
  source 1..20: mol_type = other RNA
tcgaattatactttgattga

SEQ ID NO: 353 - RNA, 20 nt, Synthetic construct
  source 1..20: mol_type = other RNA
ggaaggttctgaagaagaga

SEQ ID NO: 354 - RNA, 20 nt, Synthetic construct
  source 1..20: mol_type = other RNA
ttcaggtcttcaaacatgtc

SEQ ID NO: 355 - RNA, 20 nt, Synthetic construct
  source 1..20: mol_type = other RNA
actactgggtgtgcttggca

SEQ ID NO: 356 - RNA, 20 nt, Synthetic construct
  source 1..20: mol_type = other RNA
aacttccaagcttaccttca

SEQ ID NO: 357 - RNA, 20 nt, Synthetic construct
  source 1..20: mol_type = other RNA
cgtgagtttcccagaagaag

SEQ ID NO: 358 - RNA, 20 nt, Synthetic construct
  source 1..20: mol_type = other RNA
tttgattgagggcgtcattc

SEQ ID NO: 359 - RNA, 20 nt, Synthetic construct
  source 1..20: mol_type = other RNA
atccatcactgatgatgacc

SEQ ID NO: 360 - RNA, 20 nt, Synthetic construct
  source 1..20: mol_type = other RNA
ttcccagaagaagaggaggt

SEQ ID NO: 361 - RNA, 20 nt, Synthetic construct
  source 1..20: mol_type = other RNA
gctctccttgaaggtaagct

SEQ ID NO: 362 - RNA, 20 nt, Synthetic construct
  source 1..20: mol_type = other RNA
actgggtgtgcttggcaggg

SEQ ID NO: 363 - RNA, 20 nt, Synthetic construct
  source 1..20: mol_type = other RNA
gggtgcttataagtcatcaa

SEQ ID NO: 364 - RNA, 20 nt, Synthetic construct
  source 1..20: mol_type = other RNA
tgatcatctgtctctgaatc

SEQ ID NO: 365 - RNA, 20 nt, Synthetic construct
  source 1..20: mol_type = other RNA
cagaagaactgttacaggta

SEQ ID NO: 366 - RNA, 20 nt, Synthetic construct
  source 1..20: mol_type = other RNA
aagacctgaagaactgttac

SEQ ID NO: 367 - RNA, 20 nt, Synthetic construct
  source 1..20: mol_type = other RNA
taccaccatgctctccttga

SEQ ID NO: 368 - RNA, 20 nt, Synthetic construct
  source 1..20: mol_type = other RNA
gcaagactactgggtgtgct

SEQ ID NO: 369 - RNA, 20 nt, Synthetic construct
  source 1..20: mol_type = other RNA
attgatccatgcagccttca

SEQ ID NO: 370 - RNA, 20 nt, Synthetic construct
  source 1..20: mol_type = other RNA
caatgactcagaggaaggta

SEQ ID NO: 371 - RNA, 20 nt, Synthetic construct
  source 1..20: mol_type = other RNA
ctgaccttcctctgagtcat

SEQ ID NO: 372 - RNA, 20 nt, Synthetic construct
  source 1..20: mol_type = other RNA
tatcactgactttcagatac

SEQ ID NO: 373 - RNA, 20 nt, Synthetic construct
  source 1..20: mol_type = other RNA
cccactccatgaaggctgca

SEQ ID NO: 374 - RNA, 20 nt, Synthetic construct
  source 1..20: mol_type = other RNA
ctcactacctgtgatggttt

SEQ ID NO: 375 - RNA, 20 nt, Synthetic construct
  source 1..20: mol_type = other RNA
tcactacctgtgatggtttt

SEQ ID NO: 376 - RNA, 20 nt, Synthetic construct
  source 1..20: mol_type = other RNA
cactggttggtcttcatctt

SEQ ID NO: 377 - RNA, 20 nt, Synthetic construct
  source 1..20: mol_type = other RNA
cttacctgtaacagttcttc

SEQ ID NO: 378 - RNA, 20 nt, Synthetic construct
  source 1..20: mol_type = other RNA
actcattggcgatggcctcc

SEQ ID NO: 379 - RNA, 20 nt, Synthetic construct
  source 1..20: mol_type = other RNA
tgccataaagcaagactact

SEQ ID NO: 380 - RNA, 20 nt, Synthetic construct
  source 1..20: mol_type = other RNA
gactactgggtgtgcttggc

SEQ ID NO: 381 - RNA, 20 nt, Synthetic construct
  source 1..20: mol_type = other RNA
caactgaccttcagcagcac

SEQ ID NO: 382 - RNA, 20 nt, Synthetic construct
  source 1..20: mol_type = other RNA
caactgggtgtgcttggcag

SEQ ID NO: 383 - RNA, 20 nt, Synthetic construct
  source 1..20: mol_type = other RNA
tactgggtgtgcttggcagg

SEQ ID NO: 384 - RNA, 20 nt, Synthetic construct
  source 1..20: mol_type = other RNA
gcactggttggtcttcatct

SEQ ID NO: 385 - RNA, 20 nt, Synthetic construct
  source 1..20: mol_type = other RNA
gaccaacctcctcttcttct

SEQ ID NO: 386 - RNA, 20 nt, Synthetic construct
  source 1..20: mol_type = other RNA
gtgatggttttgggtatctc

SEQ ID NO: 387 - RNA, 20 nt, Synthetic construct
  source 1..20: mol_type = other RNA
agaccaacctcctcttcttc

SEQ ID NO: 388 - RNA, 20 nt, Synthetic construct
  source 1..20: mol_type = other RNA
cttcgacacatgggataacg

SEQ ID NO: 389 - RNA, 20 nt, Synthetic construct
  source 1..20: mol_type = other RNA
gtgcagttcagtgatcgtac

SEQ ID NO: 390 - RNA, 20 nt, Synthetic construct
  source 1..20: mol_type = other RNA
catggccacaacaactgacg

SEQ ID NO: 391 - RNA, 20 nt, Synthetic construct
  source 1..20: mol_type = other RNA
ggtggtcggagattcgtagc

SEQ ID NO: 392 - RNA, 20 nt, Synthetic construct
  source 1..20: mol_type = other RNA
ggactcacagcaaaaaagct

SEQ ID NO: 393 - RNA, 20 nt, Synthetic construct
  source 1..20: mol_type = other RNA
acctatcttcttcgacacat

SEQ ID NO: 394 - RNA, 20 nt, Synthetic construct
  source 1..20: mol_type = other RNA
aggtgctgatgtaccagttg

SEQ ID NO: 395 - RNA, 20 nt, Synthetic construct
  source 1..20: mol_type = other RNA
cttatcatctttcaacacgc

SEQ ID NO: 396 - RNA, 20 nt, Synthetic construct
  source 1..20: mol_type = other RNA
tccgaccaccactacagcaa

SEQ ID NO: 397 - RNA, 19 nt, Synthetic construct
  source 1..19: mol_type = other RNA
gtaatagccatcatttcac

SEQ ID NO: 398 - RNA, 20 nt, Synthetic construct
  source 1..20: mol_type = other RNA
gaggtgctgatgtaccagtt

SEQ ID NO: 399 - RNA, 20 nt, Synthetic construct
  source 1..20: mol_type = other RNA
ctgaaagctctccacctcca

SEQ ID NO: 400 - RNA, 20 nt, Synthetic construct
  source 1..20: mol_type = other RNA
aacctatcttcttcgacaca

SEQ ID NO: 401 - RNA, 18 nt, Synthetic construct
  source 1..18: mol_type = other RNA
tttttaacagtggcaatg

SEQ ID NO: 402 - RNA, 20 nt, Synthetic construct
  source 1..20: mol_type = other RNA
ttctccatgtcctttgtaca

SEQ ID NO: 403 - RNA, 20 nt, Synthetic construct
  source 1..20: mol_type = other RNA
aagtaatgacaaaatacctg

SEQ ID NO: 404 - RNA, 20 nt, Synthetic construct
  source 1..20: mol_type = other RNA
aacatgcccgtcttcctggg

SEQ ID NO: 405 - RNA, 20 nt, Synthetic construct
  source 1..20: mol_type = other RNA
ggacaggatatggagcaaca

SEQ ID NO: 406 - RNA, 20 nt, Synthetic construct
  source 1..20: mol_type = other RNA
ttaggaagacacaaattgca

SEQ ID NO: 407 - RNA, 20 nt, Synthetic construct
  source 1..20: mol_type = other RNA
gagctcaggtacttctgcca

SEQ ID NO: 408 - RNA, 19 nt, Synthetic construct
  source 1..19: mol_type = other RNA
ttctccttgtacaaaggaa

SEQ ID NO: 409 - RNA, 20 nt, Synthetic construct
  source 1..20: mol_type = other RNA
atcactgaactgcacgctcc

SEQ ID NO: 410 - RNA, 20 nt, Synthetic construct
  source 1..20: mol_type = other RNA
tgaagggaaagaaggtgctc

SEQ ID NO: 411 - RNA, 21 nt, Synthetic construct
  source 1..21: mol_type = other RNA
catctttcaacaccgcaggac

SEQ ID NO: 412 - RNA, 20 nt, Synthetic construct
  source 1..20: mol_type = other RNA
ctccgaccaccactacagca

SEQ ID NO: 413 - RNA, 20 nt, Synthetic construct
  source 1..20: mol_type = other RNA
tgataagcccactctacagc

SEQ ID NO: 414 - RNA, 20 nt, Synthetic construct
  source 1..20: mol_type = other RNA
gcaggccgcgtcagttgttg

SEQ ID NO: 415 - RNA, 20 nt, Synthetic construct
  source 1..20: mol_type = other RNA
taagcccactctacagctgg

SEQ ID NO: 416 - RNA, 20 nt, Synthetic construct
  source 1..20: mol_type = other RNA
cttacctccagctgtagagt

SEQ ID NO: 417 - RNA, 20 nt, Synthetic construct
  source 1..20: mol_type = other RNA
gctccatatcctgtccctgg

SEQ ID NO: 418 - RNA, 20 nt, Synthetic construct
  source 1..20: mol_type = other RNA
tttcccttcatctttgaaga

SEQ ID NO: 419 - RNA, 20 nt, Synthetic construct
  source 1..20: mol_type = other RNA
gcccttgctgtagtggtggt

SEQ ID NO: 420 - RNA, 20 nt, Synthetic construct
  source 1..20: mol_type = other RNA
gctggatgccgccatccaga

SEQ ID NO: 421 - RNA, 20 nt, Synthetic construct
  source 1..20: mol_type = other RNA
ttcctgggagggaccaaagg

SEQ ID NO: 422 - RNA, 20 nt, Synthetic construct
  source 1..20: mol_type = other RNA
acttacctccagctgtagac

SEQ ID NO: 423 - RNA, 20 nt, Synthetic construct
  source 1..20: mol_type = other RNA
ttgtggccatggacaagctg

SEQ ID NO: 424 - RNA, 20 nt, Synthetic construct
  source 1..20: mol_type = other RNA
gaaaacatgcccgtcttcct

SEQ ID NO: 425 - RNA, 20 nt, Synthetic construct
  source 1..20: mol_type = other RNA
gggcatgttttctgcttgag

SEQ ID NO: 426 - RNA, 20 nt, Synthetic construct
  source 1..20: mol_type = other RNA
tggtgaagtcagttatatcc

SEQ ID NO: 427 - RNA, 20 nt, Synthetic construct
  source 1..20: mol_type = other RNA
tcccatgtgtcgaagaagat

SEQ ID NO: 428 - RNA, 20 nt, Synthetic construct
  source 1..20: mol_type = other RNA
tgaagcccttgctgtagtgg

SEQ ID NO: 429 - RNA, 20 nt, Synthetic construct
  source 1..20: mol_type = other RNA
tgagctcgccagtgaaatga

SEQ ID NO: 430 - RNA, 20 nt, Synthetic construct
  source 1..20: mol_type = other RNA
gatcactgaaacgcacgctc

SEQ ID NO: 431 - RNA, 20 nt, Synthetic construct
  source 1..20: mol_type = other RNA
atcatttcactggcgagctc

SEQ ID NO: 432 - RNA, 20 nt, Synthetic construct
  source 1..20: mol_type = other RNA
ttggtccctccctggaagac

SEQ ID NO: 433 - RNA, 20 nt, Synthetic construct
  source 1..20: mol_type = other RNA
gacctctgccctctggatgg

SEQ ID NO: 434 - RNA, 20 nt, Synthetic construct
  source 1..20: mol_type = other RNA
actaccttcttcaaagatga

SEQ ID NO: 435 - RNA, 20 nt, Synthetic construct
  source 1..20: mol_type = other RNA
gtgaaatgatggcttattac

SEQ ID NO: 436 - RNA, 20 nt, Synthetic construct
  source 1..20: mol_type = other RNA
agcttttttgctgtgagtcc

SEQ ID NO: 437 - RNA, 20 nt, Synthetic construct
  source 1..20: mol_type = other RNA
acatgcccgtcttcctggga

SEQ ID NO: 438 - RNA, 20 nt, Synthetic construct
  source 1..20: mol_type = other RNA
ggtacagattcttttccttg

SEQ ID NO: 439 - RNA, 20 nt, Synthetic construct
  source 1..20: mol_type = other RNA
agctggatgccgccatccag

SEQ ID NO: 440 - RNA, 20 nt, Synthetic construct
  source 1..20: mol_type = other RNA
aggtcctggaaggagcactg

SEQ ID NO: 441 - RNA, 20 nt, Synthetic construct
  source 1..20: mol_type = other RNA
agaggtgctgatgtaccagt

SEQ ID NO: 442 - RNA, 20 nt, Synthetic construct
  source 1..20: mol_type = other RNA
ctacagcaagggcttcaggc

SEQ ID NO: 443 - RNA, 20 nt, Synthetic construct
  source 1..20: mol_type = other RNA
tgacaaaatacctgtggcct

SEQ ID NO: 444 - RNA, 20 nt, Synthetic construct
  source 1..20: mol_type = other RNA
actgaaagctctccacctcc

SEQ ID NO: 445 - RNA, 20 nt, Synthetic construct
  source 1..20: mol_type = other RNA
actttcttctccttgtacaa

SEQ ID NO: 446 - RNA, 20 nt, Synthetic construct
  source 1..20: mol_type = other RNA
ctaccttcttcaaagatgaa

SEQ ID NO: 447 - RNA, 20 nt, Synthetic construct
  source 1..20: mol_type = other RNA
ggacaagctgaggaagatgc

SEQ ID NO: 448 - RNA, 20 nt, Synthetic construct
  source 1..20: mol_type = other RNA
tgccgccatccagagggcag

SEQ ID NO: 449 - RNA, 20 nt, Synthetic construct
  source 1..20: mol_type = other RNA
tggagagctttcagttcata

SEQ ID NO: 450 - RNA, 21 nt, Synthetic construct
  source 1..21: mol_type = other RNA
gatgtaccagttggggaacat

SEQ ID NO: 451 - RNA, 20 nt, Synthetic construct
  source 1..20: mol_type = other RNA
tccttgaggcccaaggccac

SEQ ID NO: 452 - RNA, 20 nt, Synthetic construct
  source 1..20: mol_type = other RNA
gctcaggtcattctcctgga

SEQ ID NO: 453 - RNA, 19 nt, Synthetic construct
  source 1..19: mol_type = other RNA
agtcttacttcatctgttt

SEQ ID NO: 454 - RNA, 20 nt, Synthetic construct
  source 1..20: mol_type = other RNA
caaaaaagcttggtgatgtc

SEQ ID NO: 455 - RNA, 20 nt, Synthetic construct
  source 1..20: mol_type = other RNA
agaaaacatgcccgtcttcc

SEQ ID NO: 456 - RNA, 20 nt, Synthetic construct
  source 1..20: mol_type = other RNA
attcttttccttgaggccca

SEQ ID NO: 457 - RNA, 20 nt, Synthetic construct
  source 1..20: mol_type = other RNA
catcttcctcagcttgtcca

SEQ ID NO: 458 - RNA, 20 nt, Synthetic construct
  source 1..20: mol_type = other RNA
gttgctccatatcctgtccc

SEQ ID NO: 459 - RNA, 20 nt, Synthetic construct
  source 1..20: mol_type = other RNA
tcattctcctggaaggtctg

SEQ ID NO: 460 - RNA, 20 nt, Synthetic construct
  source 1..20: mol_type = other RNA
cattctcctggaaggtctgt

SEQ ID NO: 461 - RNA, 20 nt, Synthetic construct
  source 1..20: mol_type = other RNA
ctctccgcagtgctccttcc

SEQ ID NO: 462 - RNA, 20 nt, Synthetic construct
  source 1..20: mol_type = other RNA
tgatggccctaaacagatga

SEQ ID NO: 463 - RNA, 20 nt, Synthetic construct
  source 1..20: mol_type = other RNA
aggtgctcaggtcattctcc

SEQ ID NO: 464 - RNA, 20 nt, Synthetic construct
  source 1..20: mol_type = other RNA
aaattacccaaagaagaaga

SEQ ID NO: 465 - RNA, 20 nt, Synthetic construct
  source 1..20: mol_type = other RNA
ttcaaagatgaagggaaaga

SEQ ID NO: 466 - RNA, 20 nt, Synthetic construct
  source 1..20: mol_type = other RNA
ctggacctctgccctctgga

SEQ ID NO: 467 - RNA, 20 nt, Synthetic construct
  source 1..20: mol_type = other RNA
gatagaaatcaataacaagc

SEQ ID NO: 468 - RNA, 20 nt, Synthetic construct
  source 1..20: mol_type = other RNA
accactacagcaagggcttc

SEQ ID NO: 469 - RNA, 20 nt, Synthetic construct
  source 1..20: mol_type = other RNA
agtctgcccagttccccaac

SEQ ID NO: 470 - RNA, 19 nt, Synthetic construct
  source 1..19: mol_type = other RNA
tcctggaggtctgtgggca

SEQ ID NO: 471 - RNA, 20 nt, Synthetic construct
  source 1..20: mol_type = other RNA
gtcttcctgggagggaccaa

SEQ ID NO: 472 - RNA, 20 nt, Synthetic construct
  source 1..20: mol_type = other RNA
tgatgtaccagttggggaac

SEQ ID NO: 473 - RNA, 20 nt, Synthetic construct
  source 1..20: mol_type = other RNA
gtcttaccttcatctgttta

SEQ ID NO: 474 - RNA, 20 nt, Synthetic construct
  source 1..20: mol_type = other RNA
acctgtggccttgggcctca

SEQ ID NO: 475 - RNA, 20 nt, Synthetic construct
  source 1..20: mol_type = other RNA
tttggtccctcccaggaaga

SEQ ID NO: 476 - RNA, 20 nt, Synthetic construct
  source 1..20: mol_type = other RNA
gcctgaagcccttgctgtag

SEQ ID NO: 477 - RNA, 20 nt, Synthetic construct
  source 1..20: mol_type = other RNA
gcagaggtccaggtcctgga

SEQ ID NO: 478 - RNA, 20 nt, Synthetic construct
  source 1..20: mol_type = other RNA
ccacctccagggacaggata

SEQ ID NO: 479 - RNA, 20 nt, Synthetic construct
  source 1..20: mol_type = other RNA
agctctccacctccagggac

SEQ ID NO: 480 - RNA, 20 nt, Synthetic construct
  source 1..20: mol_type = other RNA
tccctgcccacagaccttcc

SEQ ID NO: 481 - RNA, 20 nt, Synthetic construct
  source 1..20: mol_type = other RNA
gcagtgctccttccaggacc

SEQ ID NO: 482 - RNA, 20 nt, synthetic construct
  source 1..20: mol_type = other RNA
ccatatcctgtccctggagg

SEQ ID NO: 483 - RNA, 20 nt, Synthetic construct
  source 1..20: mol_type = other RNA
gagaaaatacctgtggcctt

SEQ ID NO: 484 - RNA, 20 nt, Synthetic construct
  source 1..20: mol_type = other RNA
ctcctggaaggtctgtgggc

SEQ ID NO: 485 - RNA, 20 nt, Synthetic construct
  source 1..20: mol_type = other RNA
cgcgtcagttgttgtggcca

SEQ ID NO: 486 - RNA, 20 nt, Synthetic construct
  source 1..20: mol_type = other RNA
agttatatcctggccgcctt

SEQ ID NO: 487 - RNA, 20 nt, Synthetic construct
  source 1..20: mol_type = other RNA
ggccgcctttggtccctccc

SEQ ID NO: 488 - RNA, 20 nt, Synthetic construct
  source 1..20: mol_type = other RNA
catccagagggcagaggtcc

SEQ ID NO: 489 - RNA, 20 nt, Synthetic construct
  source 1..20: mol_type = other RNA
gagggcagaggtccaggtcc

SEQ ID NO: 490 - RNA, 20 nt, Synthetic construct
  source 1..20: mol_type = other RNA
gggagggaccaaaggcggcc

SEQ ID NO: 491 - RNA, 20 nt, Synthetic construct
  source 1..20: mol_type = other RNA
accttcttctttgaagctga

SEQ ID NO: 492 - RNA, 20 nt, Synthetic construct
  source 1..20: mol_type = other RNA
cgcttttccatcttcttctt

SEQ ID NO: 493 - RNA, 20 nt, Synthetic construct
  source 1..20: mol_type = other RNA
ggacctggacctctgccctc

SEQ ID NO: 494 - RNA, 18 nt, Synthetic construct
  source 1..18: mol_type = other RNA
ccttctttgggtaatttt

SEQ ID NO: 495 - RNA, 20 nt, Synthetic construct
  source 1..20: mol_type = other RNA
gcttttccatcttcttcttt

SEQ ID NO: 496 - RNA, 20 nt, Synthetic construct
  source 1..20: mol_type = other RNA
ttcttctttgggtaattttt

SEQ ID NO: 497 - RNA, 20 nt, Synthetic construct
  source 1..20: mol_type = other RNA
gaaatgtgaaggtgagacca

SEQ ID NO: 498 - RNA, 20 nt, Synthetic construct
  source 1..20: mol_type = other RNA
tgatggccctggaaatgtga

SEQ ID NO: 499 - RNA, 20 nt, Synthetic construct
  source 1..20: mol_type = other RNA
ggtctcaccttcacatttcc

SEQ ID NO: 500 - RNA, 20 nt, Synthetic construct
  source 1..20: mol_type = other RNA
gtctcaccttcacatttcca

SEQ ID NO: 501 - RNA, 20 nt, Synthetic construct
  source 1..20: mol_type = other RNA
aaatgtgaaggtgagaccat

SEQ ID NO: 502 - RNA, 20 nt, Synthetic construct
  source 1..20: mol_type = other RNA
gacctattctttgaagctga

SEQ ID NO: 503 - RNA, 20 nt, Synthetic construct
  source 1..20: mol_type = other RNA
ttctttgaagctgatggccc

SEQ ID NO: 504 - RNA, 20 nt, Synthetic construct
  source 1..20: mol_type = other RNA
ggccatcagcttcaaagaat

SEQ ID NO: 505 - RNA, 20 nt, Synthetic construct
  source 1..20: mol_type = other RNA
cgtgtcagtcattgtagctt

SEQ ID NO: 506 - RNA, 20 nt, Synthetic construct
  source 1..20: mol_type = other RNA
actcttgttacagagctggt

SEQ ID NO: 507 - RNA, 20 nt, Synthetic construct
  source 1..20: mol_type = other RNA
cctcatctaccagagaactg

SEQ ID NO: 508 - RNA, 20 nt, Synthetic construct
  source 1..20: mol_type = other RNA
agacctgaaccacagttctc

SEQ ID NO: 509 - RNA, 20 nt, Synthetic construct
  source 1..20: mol_type = other RNA
gctggtgggagacttgcaac

SEQ ID NO: 510 - RNA, 20 nt, Synthetic construct
  source 1..20: mol_type = other RNA
cacagttctctggtagatga

SEQ ID NO: 511 - RNA, 20 nt, Synthetic construct
  source 1..20: mol_type = other RNA
aggtcttggcagcagcactg

SEQ ID NO: 512 - RNA, 20 nt, Synthetic construct
  source 1..20: mol_type = other RNA
tcagactcttgttacagagc

SEQ ID NO: 513 - RNA, 20 nt, Synthetic construct
  source 1..20: mol_type = other RNA
agctctgtaacaagagtctg

SEQ ID NO: 514 - RNA, 20 nt, Synthetic construct
  source 1..20: mol_type = other RNA
gagaactgtggttcaggtct

SEQ ID NO: 515 - RNA, 20 nt, Synthetic construct
  source 1..20: mol_type = other RNA
gcagcactgtggagagacca

SEQ ID NO: 516 - RNA, 20 nt, Synthetic construct
  source 1..20: mol_type = other RNA
ccacagttctctggtagatg

SEQ ID NO: 517 - RNA, 20 nt, Synthetic construct
  source 1..20: mol_type = other RNA
ctaccagagaactgtggttc

SEQ ID NO: 518 - RNA, 21 nt, Synthetic construct
  source 1..21: mol_type = other RNA
catcctccttggaggacctgt

SEQ ID NO: 519 - RNA, 20 nt, Synthetic construct
  source 1..20: mol_type = other RNA
gactcttgttacagagctgg

SEQ ID NO: 520 - RNA, 20 nt, Synthetic construct
  source 1..20: mol_type = other RNA
ttttgctacatctttgaaga

SEQ ID NO: 521 - RNA, 20 nt, Synthetic construct
  source 1..20: mol_type = other RNA
tgtagcaaaagatgctcttc

SEQ ID NO: 522 - RNA, 20 nt, Synthetic construct
  source 1..20: mol_type = other RNA
tcctggaggacctgtgggca

SEQ ID NO: 523 - RNA, 20 nt, Synthetic construct
  source 1..20: mol_type = other RNA
gctgaagaagccctgcccac

SEQ ID NO: 524 - RNA, 20 nt, Synthetic construct
  source 1..20: mol_type = other RNA
tgctcttcaggtcatcctcc

SEQ ID NO: 525 - RNA, 20 nt, Synthetic construct
  source 1..20: mol_type = other RNA
ctcctggaggacctgtgggc

SEQ ID NO: 526 - RNA, 20 nt, Synthetic construct
  source 1..20: mol_type = other RNA
ctgcccacaggtcctccagg

SEQ ID NO: 527 - RNA, 20 nt, Synthetic construct
  source 1..20: mol_type = other RNA
tcatcctcctggaggacctg

SEQ ID NO: 528 - RNA, 20 nt, Synthetic construct
  source 1..20: mol_type = other RNA
tcttcaggtcatcctcctgg

SEQ ID NO: 529 - RNA, 20 nt, Synthetic construct
  source 1..20: mol_type = other RNA
gccctgcccacaggtcctcc

SEQ ID NO: 530 - RNA, 20 nt, Synthetic construct
  source 1..20: mol_type = other RNA
tgatgcagccatgcaatcgg

SEQ ID NO: 531 - RNA, 20 nt, Synthetic construct
  source 1..20: mol_type = other RNA
cttgcagtccaccgattgca

SEQ ID NO: 532 - RNA, 20 nt, Synthetic construct
  source 1..20: mol_type = other RNA
atcggtggactgcaagttac

SEQ ID NO: 533 - RNA, 21 nt, Synthetic construct
  source 1..21: mol_type = other RNA
gtgcaacaaacaaggtaacgg

SEQ ID NO: 534 - RNA, 20 nt, Synthetic construct
  source 1..20: mol_type = other RNA
cttcgggctctccacctcaa

SEQ ID NO: 535 - RNA, 20 nt, Synthetic construct
  source 1..20: mol_type = other RNA
tcgggctctccacctcaatg

SEQ ID NO: 536 - RNA, 20 nt, Synthetic construct
  source 1..20: mol_type = other RNA
ggacataagccacaaatacc

SEQ ID NO: 537 - RNA, 19 nt, Synthetic construct
  source 1..19: mol_type = other RNA
tagacagcaaaggtatttg

SEQ ID NO: 538 - RNA, 20 nt, Synthetic construct
  source 1..20: mol_type = other RNA
gagtgatgcagccatgcaat

SEQ ID NO: 539 - RNA, 20 nt, Synthetic construct
  source 1..20: mol_type = other RNA
ttcgggctctccacctcaat

SEQ ID NO: 540 - RNA, 20 nt, Synthetic construct
  source 1..20: mol_type = other RNA
tgtgaacaaacaaggtaacg

SEQ ID NO: 541 - RNA, 20 nt, Synthetic construct
  source 1..20: mol_type = other RNA
atgtgaacaaacaaggtaac

SEQ ID NO: 542 - RNA, 20 nt, Synthetic construct
  source 1..20: mol_type = other RNA
ggggaaaatgtgaacaaaca

SEQ ID NO: 543 - RNA, 20 nt, Synthetic construct
  source 1..20: mol_type = other RNA
gtctaactcatatgagcttc

SEQ ID NO: 544 - RNA, 20 nt, Synthetic construct
  source 1..20: mol_type = other RNA
catatggattagacagcacc

SEQ ID NO: 545 - RNA, 20 nt, Synthetic construct
  source 1..20: mol_type = other RNA
tgttcacattttccccattg

SEQ ID NO: 546 - RNA, 20 nt, Synthetic construct
  source 1..20: mol_type = other RNA
tgtctaactcatctgagctt

SEQ ID NO: 547 - RNA, 20 nt, Synthetic construct
  source 1..20: mol_type = other RNA
agatgataggttctgaaatg

SEQ ID NO: 548 - RNA, 20 nt, Synthetic construct
  source 1..20: mol_type = other RNA
gcatctgttttgcagatgat

SEQ ID NO: 549 - RNA, 21 nt, Synthetic construct
  source 1..21: mol_type = other RNA
tcaccattttccccattgagg

SEQ ID NO: 550 - RNA, 20 nt, Synthetic construct
  source 1..20: mol_type = other RNA
aatgtgaacaaacaaggtaa

SEQ ID NO: 551 - RNA, 20 nt, Synthetic construct
  source 1..20: mol_type = other RNA
tatcatctgcaaaacagatg

SEQ ID NO: 552 - RNA, 20 nt, Synthetic construct
  source 1..20: mol_type = other RNA
tgaccatctctctctgaatc

SEQ ID NO: 553 - RNA, 20 nt, Synthetic construct
  source 1..20: mol_type = other RNA
ttacctgattcagagagaga

SEQ ID NO: 554 - RNA, 20 nt, Synthetic construct
  source 1..20: mol_type = other RNA
gacatcccagcttaccttca

SEQ ID NO: 555 - RNA, 20 nt, Synthetic construct
  source 1..20: mol_type = other RNA
gtcacagctcatatcataga

SEQ ID NO: 556 - RNA, 20 nt, Synthetic construct
  source 1..20: mol_type = other RNA
atgacacagaagaaggtaag

SEQ ID NO: 557 - RNA, 20 nt, Synthetic construct
  source 1..20: mol_type = other RNA
cactaccacattttccttga

SEQ ID NO: 558 - RNA, 20 nt, Synthetic construct
  source 1..20: mol_type = other RNA
aatgtcttccaggtcatcat

SEQ ID NO: 559 - RNA, 20 nt, Synthetic construct
  source 1..20: mol_type = other RNA
tggtagtggtggcagccaat

SEQ ID NO: 560 - RNA, 20 nt, Synthetic construct
  source 1..20: mol_type = other RNA
atcatagaaggatttctatg

SEQ ID NO: 561 - RNA, 20 nt, Synthetic construct
  source 1..20: mol_type = other RNA
ggatgtctttgagatttcag

SEQ ID NO: 562 - RNA, 20 nt, Synthetic construct
  source 1..20: mol_type = other RNA
cattttccttgaaggtaagc

SEQ ID NO: 563 - RNA, 20 nt, Synthetic construct
  source 1..20: mol_type = other RNA
attttccttgaaggtaagct

SEQ ID NO: 564 - RNA, 20 nt, Synthetic construct
  source 1..20: mol_type = other RNA
acatgcagtcctcatgaagt

SEQ ID NO: 565 - RNA, 20 nt, Synthetic construct
  source 1..20: mol_type = other RNA
tgtcattggcaatgtcttcc

SEQ ID NO: 566 - RNA, 20 nt, Synthetic construct
  source 1..20: mol_type = other RNA
gtggtagtggtggcagccaa

SEQ ID NO: 567 - RNA, 20 nt, Synthetic construct
  source 1..20: mol_type = other RNA
gagctgtgacccacttcatg

SEQ ID NO: 568 - RNA, 20 nt, Synthetic construct
  source 1..20: mol_type = other RNA
cttaccttcttctgtgtcat

SEQ ID NO: 569 - RNA, 20 nt, Synthetic construct
  source 1..20: mol_type = other RNA
tagaaggatttctaggagga

SEQ ID NO: 570 - RNA, 20 nt, Synthetic construct
  source 1..20: mol_type = other RNA
gcttaccttcaaggaaaatg

SEQ ID NO: 571 - RNA, 20 nt, Synthetic construct
  source 1..20: mol_type = other RNA
ggaacattctaaagaagaga

SEQ ID NO: 572 - RNA, 20 nt, Synthetic construct
  source 1..20: mol_type = other RNA
attcatcaccgatgatgacc

SEQ ID NO: 573 - RNA, 20 nt, Synthetic construct
  source 1..20: mol_type = other RNA
cttctttagaatcttcccat

SEQ ID NO: 574 - RNA, 20 nt, Synthetic construct
  source 1..20: mol_type = other RNA
gacatgcagtcctcatgaag

SEQ ID NO: 575 - RNA, 20 nt, Synthetic construct
  source 1..20: mol_type = other RNA
attgccaatgacacagaaga

SEQ ID NO: 576 - RNA, 20 nt, Synthetic construct
  source 1..20: mol_type = other RNA
cttcaaggaaaatgtggtag

SEQ ID NO: 577 - RNA, 20 nt, Synthetic construct
  source 1..20: mol_type = other RNA
caaggaaaatgtggtagtgg

SEQ ID NO: 578 - RNA, 20 nt, Synthetic construct
  source 1..20: mol_type = other RNA
agtatagttcgacaaacagg

SEQ ID NO: 579 - RNA, 20 nt, Synthetic construct
  source 1..20: mol_type = other RNA
tctgtaatgcagcagtcatg

SEQ ID NO: 580 - RNA, 20 nt, Synthetic construct
  source 1..20: mol_type = other RNA
ttacagaatttggatgatgc

SEQ ID NO: 581 - RNA, 20 nt, Synthetic construct
  source 1..20: mol_type = other RNA
gtcgaacaatactttgattg

SEQ ID NO: 582 - RNA, 20 nt, Synthetic construct
  source 1..20: mol_type = other RNA
aagttgtatgctactgatct

SEQ ID NO: 583 - RNA, 20 nt, Synthetic construct
  source 1..20: mol_type = other RNA
caaagtatagttcgacaaac

SEQ ID NO: 584 - RNA, 20 nt, Synthetic construct
  source 1..20: mol_type = other RNA
gttgtcattcaggatgaatt

SEQ ID NO: 585 - RNA, 20 nt, Synthetic construct
  source 1..20: mol_type = other RNA
atgaaaaatacaactatata

SEQ ID NO: 586 - RNA, 20 nt, Synthetic construct
  source 1..20: mol_type = other RNA
gaagttgtatgctactgatc

SEQ ID NO: 587 - RNA, 20 nt, Synthetic construct
  source 1..20: mol_type = other RNA
ggttgtcattcaggatgaat

SEQ ID NO: 588 - RNA, 20 nt, Synthetic construct
  source 1..20: mol_type = other RNA
tttgattgaggttgtcattc

SEQ ID NO: 589 - RNA, 20 nt, Synthetic construct
  source 1..20: mol_type = other RNA
agttgtatttttcattgtta

SEQ ID NO: 590 - RNA, 20 nt, Synthetic construct
  source 1..20: mol_type = other RNA
gactgctgcattacagaatt

SEQ ID NO: 591 - RNA, 36 nt, Mus musculus
  source 1..36: mol_type = other RNA
ggactgccctctatgacagacttccagatatcataa

SEQ ID NO: 592 - RNA, 48 nt, Homo sapiens
  source 1..48: mol_type = other RNA
gggccaccctctatcactgactttcagatactggaaaaccaggcgtag

SEQ ID NO: 593 - RNA, 48 nt, Equus caballus
  source 1..48: mol_type = other RNA
gggcaaccctctatcactgactttcagatattggacaaccagttttga

SEQ ID NO: 594 - RNA, 48 nt, Felis catus
  source 1..48: mol_type = other RNA
ggactaccctctgtcactgactttcagatactggaaacccagtcttga

SEQ ID NO: 595 - RNA, 48 nt, Canis sp.
  source 1..48: mol_type = other RNA
gggcaaccctctatcactgactttcggttactggaaacccagccttga

SEQ ID NO: 596 - RNA, 41 nt, Mus musculus
  source 1..41: mol_type = other RNA
aggacataattgacttcaccatggaatccgtgtcttcctaa

SEQ ID NO: 597 - RNA, 46 nt, Homo sapiens
  source 1..46: mol_type = other RNA
cggccaggatataactgacttcaccatgcaatttgtgtcttcctaa

SEQ ID NO: 598 - RNA, 43 nt, Canis sp.
  source 1..43: mol_type = other RNA
tggccaggatataactgacttcaccatggaattctcttcctag

SEQ ID NO: 599 - RNA, 42 nt, Felis catus
  source 1..42: mol_type = other RNA
tggtcaggatataactgacttcatcatggaaagcgcttccta

SEQ ID NO: 600 - RNA, 46 nt, Equus caballus
  source 1..46: mol_type = other RNA
cggccgggacataactgacttcatcatggaaatcacctctgcctaa

SEQ ID NO: 601 - RNA, 53 nt, Synthetic construct
  source 1..53: mol_type = other RNA
  misc_difference 26^27: replace = a
tacctgattcagagacagatgatcaaatggaggaactgtcttcttcattttca

SEQ ID NO: 602 - RNA, 53 nt, Synthetic construct
  source 1..53: mol_type = other RNA
  misc_difference 26^27: replace = t
atggagtgggccatagcttacatgattagaaggatttctgtgaggaaggaaaa

SEQ ID NO: 603 - RNA, 53 nt, Synthetic construct; identical to SEQ ID NO: 601
  source 1..53: mol_type = other RNA
  misc_difference 26^27: replace = a

SEQ ID NO: 604 - RNA, 53 nt, Synthetic construct; identical to SEQ ID NO: 602
  source 1..53: mol_type = other RNA
  misc_difference 26^27: replace = t

SEQ ID NO: 605 - RNA, 53 nt, Synthetic construct
  source 1..53: mol_type = other RNA
  misc_difference 26^27: replace = a
ctttgaagctgatggccctaaacagaacgaaggtaagactatgggcccaactc

SEQ ID NO: 606 - RNA, 53 nt, Synthetic construct
  source 1..53: mol_type = other RNA
  misc_difference 26^27: replace = t
tgctgtagtggtggtcggagattcgttagctggatgccgccatccagagggca

SEQ ID NO: 607 - RNA, 53 nt, Synthetic construct
  source 1..53: mol_type = other RNA
  misc_difference 26^27: replace = a
catttcagaacctatcttcttcgacaacatgggataacgaggcttatgtgcac

SEQ ID NO: 608 - RNA, 53 nt, Synthetic construct
  source 1..53: mol_type = other RNA
  misc_difference 26^27: replace = a
acctatcttcttcgacacatgggataaacgaggcttatgtgcacgatgcacct

SEQ ID NO: 609 - RNA, 53 nt, Synthetic construct
  source 1..53: mol_type = other RNA
  misc_difference 26^27: replace = t
tcccggagcgtccagttcagtgatcgttacaggtgcatcgtgcacataagcct

SEQ ID NO: 610 - RNA, 53 nt, Synthetic construct
  source 1..53: mol_type = other RNA
  misc_difference 26^27: replace = a
ttctgaaattgaccatctctctctgaaatcaggtaagtaaatgactgcaattc

SEQ ID NO: 611 - RNA, 53 nt, Synthetic construct
  source 1..53: mol_type = other RNA
  misc_difference 26^27: replace = t
aatctcaaagacatcccagcttacctttcaaggaaaatgtggtagtggtggca

SEQ ID NO: 612 - RNA, 53 nt, Synthetic construct
  source 1..53: mol_type = other RNA
  misc_difference 26^27: replace = c
ctcaatcaaagtatagttcgacaaaccaggaggaaattacctcatgactgctg

SEQ ID NO: 613 - RNA, 52 nt, Synthetic construct
  source 1..52: mol_type = other RNA
  misc_difference 25^26: replace = c
catccaaattcttaatgcagcagtccatgaggtaatttcctcctgtttgtcg

SEQ ID NO: 614 - RNA, 53 nt, Synthetic construct; identical to SEQ ID NO: 610
  source 1..53: mol_type = other RNA
  misc_difference 26^27: replace = a

SEQ ID NO: 615 - RNA, 53 nt, Synthetic construct; identical to SEQ ID NO: 611
  source 1..53: mol_type = other RNA
  misc_difference 26^27: replace = t

SEQ ID NO: 616 - RNA, 53 nt, Synthetic construct
  source 1..53: mol_type = other RNA
  misc_difference 26^27: replace = t
ctttgaagctgatggccctggaaatgttgaaggtgagaccatgggcttagctc

SEQ ID NO: 617 - RNA, 53 nt, Synthetic construct
  source 1..53: mol_type = other RNA
  misc_difference 26^27: replace = t
atgcctcagactcttgttacagacgttggtgggagacttgcaactggatgccc

SEQ ID NO: 618 - RNA, 53 nt, Synthetic construct; identical to SEQ ID NO: 616
  source 1..53: mol_type = other RNA
  misc_difference 26^27: replace = t

SEQ ID NO: 619 - RNA, 53 nt, Synthetic construct; identical to SEQ ID NO: 617
  source 1..53: mol_type = other RNA
  misc_difference 26^27: replace = t

SEQ ID NO: 620 - DNA, 20 bp, Synthetic construct
  source 1..20: mol_type = other DNA
cattgggaggatgcttagga

SEQ ID NO: 621 - DNA, 20 bp, Synthetic construct
  source 1..20: mol_type = other DNA
ggctgctttctctccaacag

SEQ ID NO: 622 - DNA, 20 bp, Synthetic construct
  source 1..20: mol_type = other DNA
aggaagcctgtgtctggttg

SEQ ID NO: 623 - DNA, 20 bp, Synthetic construct
  source 1..20: mol_type = other DNA
tggcatcgtgagataagctg

SEQ ID NO: 624 - DNA, 20 bp, Synthetic construct
  source 1..20: mol_type = other DNA
tggtttcaggaaaacccaag

SEQ ID NO: 625 - DNA, 20 bp, Synthetic construct
  source 1..20: mol_type = other DNA
gcagtatggccaagaaagga

SEQ ID NO: 626 - DNA, 20 bp, Synthetic construct
  source 1..20: mol_type = other DNA
ctgggcaagaacattggatt

SEQ ID NO: 627 - RNA, 20 nt, Homo sapiens
  source 1..20: mol_type = other RNA
agctggatgccgccatccag

SEQ ID NO: 628 - RNA, 20 nt, Homo sapiens
  source 1..20: mol_type = other RNA
accactacagcaagggcttc

SEQ ID NO: 629 - RNA, 20 nt, Homo sapiens
  source 1..20: mol_type = other RNA
catggccacaacaactgacg

SEQ ID NO: 630 - RNA, 20 nt, Homo sapiens
  source 1..20: mol_type = other RNA
ggtggtcggagattcgtagc

SEQ ID NO: 631 - RNA, 20 nt, Homo sapiens
  source 1..20: mol_type = other RNA
ctacagcaagggcttcaggc

SEQ ID NO: 632 - RNA, 20 nt, Homo sapiens
  source 1..20: mol_type = other RNA
gacctctgccctctggatgg

SEQ ID NO: 633 - RNA, 20 nt, Homo sapiens
  source 1..20: mol_type = other RNA
ctctccgcagtgctccttcc

SEQ ID NO: 634 - RNA, 20 nt, Homo sapiens
  source 1..20: mol_type = other RNA
cattctcctggaaggtctgt

SEQ ID NO: 635 - RNA, 20 nt, Homo sapiens
  source 1..20: mol_type = other RNA
atggtggtagtagcaaccaa

SEQ ID NO: 636 - RNA, 20 nt, Homo sapiens
  source 1..20: mol_type = other RNA
gccatagcttacatgataga

SEQ ID NO: 637 - RNA, 20 nt, Homo sapiens
  source 1..20: mol_type = other RNA
cagagacagatgatcaatgg

SEQ ID NO: 638 - RNA, 20 nt, Homo sapiens
  source 1..20: mol_type = other RNA
attcagagacagatgatcaa

SEQ ID NO: 639 - RNA, 20 nt, Homo sapiens
  source 1..20: mol_type = other RNA
tggtggtagtagcaaccaac

SEQ ID NO: 640 - RNA, 20 nt, Homo sapiens
  source 1..20: mol_type = other RNA
ctccaggtcatcatcagtga

SEQ ID NO: 641 - RNA, 20 nt, Equus caballus
  source 1..20: mol_type = other RNA
tggtgctggtggcagccaac

SEQ ID NO: 642 - RNA, 20 nt, Equus caballus
  source 1..20: mol_type = other RNA
cttcttcagagtcttcccgt

SEQ ID NO: 643 - RNA, 20 nt, Equus caballus
  source 1..20: mol_type = other RNA
ttacctgagtcagagagaga

SEQ ID NO: 644 - RNA, 20 nt, Equus caballus
  source 1..20: mol_type = other RNA
aagctatgacccacttcctg

SEQ ID NO: 645 - RNA, 20 nt, Equus caballus
  source 1..20: mol_type = other RNA
ccatgcagtcctcaggaagt

SEQ ID NO: 646 - RNA, 20 nt, Canis sp.
  source 1..20: mol_type = other RNA
acatgcagtcctcatgaagt

SEQ ID NO: 647 - RNA, 20 nt, Canis sp.
  source 1..20: mol_type = other RNA
gacatcccagcttaccttca

SEQ ID NO: 648 - RNA, 20 nt, Canis sp.
  source 1..20: mol_type = other RNA
gagctgtgacccacttcatg

SEQ ID NO: 649 - RNA, 20 nt, Canis sp.
  source 1..20: mol_type = other RNA
cattttccttgaaggtaagc

SEQ ID NO: 650 - RNA, 20 nt, Canis sp.
  source 1..20: mol_type = other RNA
tggtagtggtggcagccaat

SEQ ID NO: 651 - RNA, 20 nt, Canis sp.
  source 1..20: mol_type = other RNA
attttccttgaaggtaagct

SEQ ID NO: 652 - RNA, 20 nt, Canis sp.
  source 1..20: mol_type = other RNA
cttctttagaatcttcccat

SEQ ID NO: 653 - RNA, 20 nt, Canis sp.
  source 1..20: mol_type = other RNA
ggatgtctttgagatttcag

SEQ ID NO: 654 - RNA, 20 nt, Canis sp.
  source 1..20: mol_type = other RNA
gtggtagtggtggcagccaa

SEQ ID NO: 655 - RNA, 20 nt, Canis sp.
  source 1..20: mol_type = other RNA
cactaccacattttccttga

SEQ ID NO: 656 - RNA, 20 nt, Felis catus
  source 1..20: mol_type = other RNA
ggtaagctggggtgtcttag

SEQ ID NO: 657 - RNA, 20 nt, Felis catus
  source 1..20: mol_type = other RNA
attcctcactgctgatgacc

SEQ ID NO: 658 - RNA, 20 nt, Felis catus
  source 1..20: mol_type = other RNA
tggtgatggtggcagccaat

SEQ ID NO: 659 - RNA, 20 nt, Felis catus
  source 1..20: mol_type = other RNA
cttccaggtcatcagcagtg

SEQ ID NO: 660 - RNA, 20 nt, Equus caballus
  source 1..20: mol_type = other RNA
tgaaagtcttgttgtaaagt

SEQ ID NO: 661 - RNA, 20 nt, Equus caballus
  source 1..20: mol_type = other RNA
gacctcagctccatgggcga

SEQ ID NO: 662 - RNA, 20 nt, Equus caballus
  source 1..20: mol_type = other RNA
ctggatgcccccatcgccca

SEQ ID NO: 663 - RNA, 20 nt, Equus caballus
  source 1..20: mol_type = other RNA
ccccatcgcccatggagctg

SEQ ID NO: 664 - RNA, 20 nt, Equus caballus
  source 1..20: mol_type = other RNA
aagtcttgttgtaaagttgg

SEQ ID NO: 665 - RNA, 20 nt, Canis sp.
  source 1..20: mol_type = other RNA
cgtgtcagtcattgtagctt

SEQ ID NO: 666 - RNA, 20 nt, Canis sp.
  source 1..20: mol_type = other RNA
ccacagttctctggtagatg

SEQ ID NO: 667 - RNA, 20 nt, Canis sp.
  source 1..20: mol_type = other RNA
agacctgaaccacagttctc

SEQ ID NO: 668 - RNA, 20 nt, Canis sp.
  source 1..20: mol_type = other RNA
cctcatctaccagagaactg

SEQ ID NO: 669 - RNA, 20 nt, Canis sp.
  source 1..20: mol_type = other RNA
tcagactcttgttacagagc

SEQ ID NO: 670 - RNA, 20 nt, Felis catus
  source 1..20: mol_type = other RNA
gtagtaagccatcatttcac

SEQ ID NO: 671 - RNA, 20 nt, Felis catus
  source 1..20: mol_type = other RNA
gagtcttaggcatgccgtgt

SEQ ID NO: 672 - RNA, 20 nt, Felis catus
  source 1..20: mol_type = other RNA
aaacctgagccacagttttc

SEQ ID NO: 673 - RNA, 20 nt, Felis catus
  source 1..20: mol_type = other RNA
atcatttcactggtgagttc

SEQ ID NO: 674 - RNA, 20 nt, Felis catus
  source 1..20: mol_type = other RNA
actcttgttgtcgggctggt

SEQ ID NO: 675 - RNA, 20 nt, Felis catus
  source 1..20: mol_type = other RNA
gactcttgttgtcgggctgg

SEQ ID NO: 676 - RNA, 20 nt, Felis catus
  source 1..20: mol_type = other RNA
cctcatctcccagaaaactg

SEQ ID NO: 677 - RNA, 20 nt, Felis catus
  source 1..20: mol_type = other RNA
tgagaatgacctgttctttg

SEQ ID NO: 678 - RNA, 20 nt, Felis catus
  source 1..20: mol_type = other RNA
taagactcttgttgtcgggc

SEQ ID NO: 679 - RNA, 20 nt, Felis catus
  source 1..20: mol_type = other RNA
aacctgagccacagttttct

SEQ ID NO: 680 - protein, 1434 aa, Synthetic construct
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKAAALEHHHHHH

SEQ ID NO: 681 - RNA, 20 nt, Synthetic construct
  source 1..20: mol_type = other RNA
atggtggtagtagcaaccaa

SEQ ID NO: 682 - RNA, 20 nt, Synthetic construct
  source 1..20: mol_type = other RNA
cagagacagatgatcaatgg

SEQ ID NO: 683 - RNA, 20 nt, Synthetic construct
  source 1..20: mol_type = other RNA
tatgtaatgcagcagccgtg

SEQ ID NO: 684 - RNA, 20 nt, Synthetic construct
  source 1..20: mol_type = other RNA
tggtggtagtagcaaccaac

SEQ ID NO: 685 - RNA, 20 nt, Synthetic construct
  source 1..20: mol_type = other RNA
ggccatcgccaatgactcag

SEQ ID NO: 686 - RNA, 20 nt, Synthetic construct
  source 1..20: mol_type = other RNA
agccaatgatcagtacctca

SEQ ID NO: 687 - RNA, 20 nt, Synthetic construct
  source 1..20: mol_type = other RNA
agctatggcccactccatga

SEQ ID NO: 688 - RNA, 20 nt, Synthetic construct
  source 1..20: mol_type = other RNA
taccttcaaggagagcatgg

SEQ ID NO: 689 - RNA, 20 nt, Synthetic construct
  source 1..20: mol_type = other RNA
ggtagtagcaaccaacggga

SEQ ID NO: 690 - RNA, 20 nt, Synthetic construct
  source 1..20: mol_type = other RNA
gccatagcttacatgataga

SEQ ID NO: 691 - RNA, 20 nt, Synthetic construct
  source 1..20: mol_type = other RNA
attgagagacagatgatcaa

SEQ ID NO: 692 - RNA, 20 nt, Synthetic construct
  source 1..20: mol_type = other RNA
aagctaaaaggtgctgacct

SEQ ID NO: 693 - RNA, 20 nt, Synthetic construct
  source 1..20: mol_type = other RNA
cttcttcagaaccttcccgt

SEQ ID NO: 694 - RNA, 20 nt, Synthetic construct
  source 1..20: mol_type = other RNA
acattgctcaggaagctaaa

SEQ ID NO: 695 - RNA, 20 nt, Synthetic construct
  source 1..20: mol_type = other RNA
agccgtgaggtactgatcat

SEQ ID NO: 696 - RNA, 20 nt, Synthetic construct
  source 1..20: mol_type = other RNA
gggtgcttataagtcatcaa

SEQ ID NO: 697 - RNA, 20 nt, Synthetic construct
  source 1..20: mol_type = other RNA
gcttaccttcaaggagagca

SEQ ID NO: 698 - RNA, 20 nt, Synthetic construct
  source 1..20: mol_type = other RNA
taccaccatgctctccttga

SEQ ID NO: 699 - RNA, 20 nt, Synthetic construct
  source 1..20: mol_type = other RNA
ctccaggtcatcatcagtga

SEQ ID NO: 700 - RNA, 20 nt, Synthetic construct
  source 1..20: mol_type = other RNA
atcgccaatgactcagagga

SEQ ID NO: 701 - RNA, 20 nt, Synthetic construct
  source 1..20: mol_type = other RNA
ctcgaattatactttgattg

SEQ ID NO: 702 - RNA, 20 nt, Synthetic construct
  source 1..20: mol_type = other RNA
gctctccttgaaggtaagct

SEQ ID NO: 703 - RNA, 20 nt, Synthetic construct
  source 1..20: mol_type = other RNA
catcactgatgatgacctgg

SEQ ID NO: 704 - RNA, 20 nt, Synthetic construct
  source 1..20: mol_type = other RNA
tcgaattatactttgattga

SEQ ID NO: 705 - RNA, 20 nt, Synthetic construct
  source 1..20: mol_type = other RNA
attgatccatgcagccttca

SEQ ID NO: 706 - RNA, 20 nt, Synthetic construct
  source 1..20: mol_type = other RNA
cccactccatgaaggctgca

SEQ ID NO: 707 - RNA, 20 nt, Synthetic construct
  source 1..20: mol_type = other RNA
tttgattgagggcgtcattc

SEQ ID NO: 708 - RNA, 20 nt, Synthetic construct
  source 1..20: mol_type = other RNA
tccttctatcatgtaagcta

SEQ ID NO: 709 - RNA, 20 nt, Synthetic construct
  source 1..20: mol_type = other RNA
ggtcatcatcagtgatggat

SEQ ID NO: 710 - RNA, 20 nt, Synthetic construct
  source 1..20: mol_type = other RNA
cttaccttcctctgagtcat

SEQ ID NO: 711 - RNA, 20 nt, Synthetic construct
  source 1..20: mol_type = other RNA
ggtggtcggagattcgtagc

SEQ ID NO: 712 - RNA, 20 nt, Synthetic construct
  source 1..20: mol_type = other RNA
catggccacaacaactgacg

SEQ ID NO: 713 - RNA, 20 nt, Synthetic construct
  source 1..20: mol_type = other RNA
cttcgacacatgggataacg

SEQ ID NO: 714 - RNA, 20 nt, Synthetic construct
  source 1..20: mol_type = other RNA
gtgcagttcagtgatcgtac

SEQ ID NO: 715 - RNA, 20 nt, Synthetic construct
  source 1..20: mol_type = other RNA
tgagctcgccagtgaaatga

SEQ ID NO: 716 - RNA, 20 nt, Synthetic construct
  source 1..20: mol_type = other RNA
gatcactgaactgcacgctc

SEQ ID NO: 717 - RNA, 20 nt, Synthetic construct
  source 1..20: mol_type = other RNA
acctatcttcttcgacacat

SEQ ID NO: 718 - RNA, 20 nt, Synthetic construct
  source 1..20: mol_type = other RNA
gacctctgccctctggatgg

SEQ ID NO: 719 - RNA, 20 nt, Synthetic construct
  source 1..20: mol_type = other RNA
gctggatgccgccatccaga

SEQ ID NO: 720 - RNA, 20 nt, Synthetic construct
  source 1..20: mol_type = other RNA
tccgaccaccactacagcaa

SEQ ID NO: 721 - RNA, 20 nt, Synthetic construct
  source 1..20: mol_type = other RNA
tgaagcccttgctgtagtgg

SEQ ID NO: 722 - RNA, 20 nt, Synthetic construct
  source 1..20: mol_type = other RNA
ttctccatgtcctttgtaca

SEQ ID NO: 723 - RNA, 20 nt, Synthetic construct
  source 1..20: mol_type = other RNA
cttatcatctttcaacacgc

SEQ ID NO: 724 - RNA, 20 nt, Synthetic construct
  source 1..20: mol_type = other RNA
agctggatgccgccatccag

SEQ ID NO: 725 - RNA, 19 nt, Synthetic construct
  source 1..19: mol_type = other RNA
ttgtggcatggacaagctg

SEQ ID NO: 726 - RNA, 20 nt, Synthetic construct
  source 1..20: mol_type = other RNA
gctcaggtcattctcctgga

SEQ ID NO: 727 - RNA, 20 nt, Synthetic construct
  source 1..20: mol_type = other RNA
gctccatatcctgtccctgg

SEQ ID NO: 728 - RNA, 20 nt, Synthetic construct
  source 1..20: mol_type = other RNA
ctccgaccaccactacagca

SEQ ID NO: 729 - RNA, 20 nt, Synthetic construct
  source 1..20: mol_type = other RNA
ctacagcaagggcttcaggc

SEQ ID NO: 730 - RNA, 20 nt, Synthetic construct
  source 1..20: mol_type = other RNA
aacctatcttcttcgacaca

SEQ ID NO: 731 - RNA, 20 nt, Synthetic construct
  source 1..20: mol_type = other RNA
atcactgaactgcacgctcc

SEQ ID NO: 732 - RNA, 20 nt, Synthetic construct
  source 1..20: mol_type = other RNA
gcagaggtccaggtcctgga

SEQ ID NO: 733 - RNA, 20 nt, Synthetic construct
  source 1..20: mol_type = other RNA
tcccatgtgtcgaagaagat

SEQ ID NO: 734 - RNA, 20 nt, Synthetic construct
  source 1..20: mol_type = other RNA
caaaaaagcttggtgatgtc

SEQ ID NO: 735 - RNA, 20 nt, Synthetic construct
  source 1..20: mol_type = other RNA
atcatttcactggcgagctc

SEQ ID NO: 736 - RNA, 20 nt, Synthetic construct
  source 1..20: mol_type = other RNA
gtaataagccatcatttcac

SEQ ID NO: 737 - RNA, 20 nt, Synthetic construct
  source 1..20: mol_type = other RNA
catcttcctcagcttgtcca

SEQ ID NO: 738 - RNA, 20 nt, Synthetic construct
  source 1..20: mol_type = other RNA
agcttttttgctgtgagtcc

SEQ ID NO: 739 - RNA, 20 nt, Synthetic construct
  source 1..20: mol_type = other RNA
tccttgaggcccaaggccac

SEQ ID NO: 740 - RNA, 20 nt, Synthetic construct
  source 1..20: mol_type = other RNA
taagcccactctacagctgg

SEQ ID NO: 741 - RNA, 20 nt, Canis sp.
  source 1..20: mol_type = other RNA
gtcgaactatactttgattg

SEQ ID NO: 742 - RNA, 20 nt, Canis sp.
  source 1..20: mol_type = other RNA
ttgccagtgaagtttgacat

SEQ ID NO: 743 - RNA, 20 nt, Canis sp.
  source 1..20: mol_type = other RNA
agtatagttcgacaaacagg

SEQ ID NO: 744 - RNA, 20 nt, Canis sp.
  source 1..20: mol_type = other RNA
tggtagtggtggcagccaat

SEQ ID NO: 745 - RNA, 20 nt, Canis sp.
  source 1..20: mol_type = other RNA
acatgcagtcctcatgaagt

SEQ ID NO: 746 - RNA, 20 nt, Canis sp.
  source 1..20: mol_type = other RNA
aagttgtatgctactgatct

SEQ ID NO: 747 - RNA, 20 nt, Canis sp.
  source 1..20: mol_type = other RNA
gagctgtgacccacttcatg

SEQ ID NO: 748 - RNA, 20 nt, Canis sp.
  source 1..20: mol_type = other RNA
caaagtatagttcgacaaac

SEQ ID NO: 749 - RNA, 20 nt, Canis sp.
  source 1..20: mol_type = other RNA
gtggtagtggtggcagccaa

SEQ ID NO: 750 - RNA, 20 nt, Canis sp.
  source 1..20: mol_type = other RNA
cctgaagaactgttacaggt

SEQ ID NO: 751 - RNA, 20 nt, Canis sp.
  source 1..20: mol_type = other RNA
gtcacagctcatatcataga

SEQ ID NO: 752 - RNA, 20 nt, Canis sp.
  source 1..20: mol_type = other RNA
tctgtaatgcagcagtcatg

SEQ ID NO: 753 - RNA, 20 nt, Canis sp.
  source 1..20: mol_type = other RNA
attttccttgaaggtaagct

SEQ ID NO: 754 - RNA, 20 nt, Canis sp.
  source 1..20: mol_type = other RNA
gacatgcagtcctcatgaag

SEQ ID NO: 755 - RNA, 20 nt, Canis sp.
  source 1..20: mol_type = other RNA
aatgtcttccaggtcatcat

SEQ ID NO: 756 - RNA, 20 nt, Canis sp.
  source 1..20: mol_type = other RNA
ggatgtctttgagatttcag

SEQ ID NO: 757 - RNA, 20 nt, Canis sp.
  source 1..20: mol_type = other RNA
ccactgacctttagcaatac

SEQ ID NO: 758 - RNA, 20 nt, Canis sp.
  source 1..20: mol_type = other RNA
attcatcaccgatgatgacc

SEQ ID NO: 759 - RNA, 20 nt, Canis sp.
  source 1..20: mol_type = other RNA
gctcccatgtcaaacttcac

SEQ ID NO: 760 - RNA, 20 nt, Canis sp.
  source 1..20: mol_type = other RNA
tttgccagtgaagtttgaca

SEQ ID NO: 761 - RNA, 20 nt, Canis sp.
  source 1..20: mol_type = other RNA
gactgctgcattacagaatt

SEQ ID NO: 762 - RNA, 20 nt, Canis sp.
  source 1..20: mol_type = other RNA
gacatcccagcttaccttca

SEQ ID NO: 763 - RNA, 20 nt, Canis sp.
  source 1..20: mol_type = other RNA
gcttaccttcaaggaaaatg

SEQ ID NO: 764 - RNA, 20 nt, Canis sp.
  source 1..20: mol_type = other RNA
gaagttgtatgctactgatc

SEQ ID NO: 765 - RNA, 20 nt, Canis sp.
  source 1..20: mol_type = other RNA
gttgtcattcaggatgaatt

SEQ ID NO: 766 - RNA, 20 nt, Canis sp.
  source 1..20: mol_type = other RNA
cttctttagaatcttcccat

SEQ ID NO: 767 - RNA, 20 nt, Canis sp.
  source 1..20: mol_type = other RNA
aagacctgaagaactgttac

SEQ ID NO: 768 - RNA, 20 nt, Canis sp.
  source 1..20: mol_type = other RNA
ttcaaagaggtcaggaactt

SEQ ID NO: 769 - RNA, 20 nt, Canis sp.
  source 1..20: mol_type = other RNA
tttgatattcttagagtcac

SEQ ID NO: 770 - RNA, 20 nt, Canis sp.
  source 1..20: mol_type = other RNA
agttcttcaggtcttcaaag

SEQ ID NO: 771 - RNA, 20 nt, Canis sp.
  source 1..20: mol_type = other RNA
tgatgcagccatgcaatcgg

SEQ ID NO: 772 - RNA, 20 nt, Canis sp.
  source 1..20: mol_type = other RNA
cttgcagtccaccgattgca

SEQ ID NO: 773 - RNA, 20 nt, Canis sp.
  source 1..20: mol_type = other RNA
tcgggctctccacctcaatg

SEQ ID NO: 774 - RNA, 20 nt, Canis sp.
  source 1..20: mol_type = other RNA
aaatgtgaaggtgagaccat

SEQ ID NO: 775 - RNA, 20 nt, Canis sp.
  source 1..20: mol_type = other RNA
ccacatgagctttgtgcacg

SEQ ID NO: 776 - RNA, 20 nt, Canis sp.
  source 1..20: mol_type = other RNA
cctcatctaccagagaactg

SEQ ID NO: 777 - RNA, 20 nt, Canis sp.
  source 1..20: mol_type = other RNA
actgttgttacagagctggt

SEQ ID NO: 778 - RNA, 20 nt, Canis sp.
  source 1..20: mol_type = other RNA
gagtgatgcagccatgcaat

SEQ ID NO: 779 - RNA, 20 nt, Canis sp.
  source 1..20: mol_type = other RNA
tggcttactccaggtcagtg

SEQ ID NO: 780 - RNA, 20 nt, Canis sp.
  source 1..20: mol_type = other RNA
actcacctctagctgtaggg

SEQ ID NO: 781 - RNA, 20 nt, Canis sp.
  source 1..20: mol_type = other RNA
tgatggccctggaaatgtga

SEQ ID NO: 782 - RNA, 20 nt, Canis sp.
  source 1..20: mol_type = other RNA
atcggtggactgcaagttac

SEQ ID NO: 783 - RNA, 20 nt, Canis sp.
  source 1..20: mol_type = other RNA
cacagttctctggtagatga

SEQ ID NO: 784 - RNA, 20 nt, Canis sp.
  source 1..20: mol_type = other RNA
cgaactcaccagtgaaatga

SEQ ID NO: 785 - RNA, 20 nt, Canis sp.
  source 1..20: mol_type = other RNA
ctcacctctagctgtagggt

SEQ ID NO: 786 - RNA, 20 nt, Canis sp.
  source 1..20: mol_type = other RNA
cttcgggctctccacctcaa

SEQ ID NO: 787 - RNA, 20 nt, Canis sp.
  source 1..20: mol_type = other RNA
gctggtgggagacttgcaac

SEQ ID NO: 788 - RNA, 20 nt, Canis sp.
  source 1..20: mol_type = other RNA
ttccacatgagctttgtgca

SEQ ID NO: 789 - RNA, 20 nt, Canis sp.
  source 1..20: mol_type = other RNA
aaagcccaccctacagctag

SEQ ID NO: 790 - RNA, 20 nt, Canis sp.
  source 1..20: mol_type = other RNA
catatgagttagacagcacc

SEQ ID NO: 791 - RNA, 20 nt, Canis sp.
  source 1..20: mol_type = other RNA
ctgtcctgtgtgatgaagga

SEQ ID NO: 792 - RNA, 20 nt, Canis sp.
  source 1..20: mol_type = other RNA
gaaatgtgaaggtgagacca

SEQ ID NO: 793 - RNA, 20 nt, Canis sp.
  source 1..20: mol_type = other RNA
aagtaataacaagatacctg

SEQ ID NO: 794 - RNA, 20 nt, Canis sp.
  source 1..20: mol_type = other RNA
cgtgtcagtcattgtagctt

SEQ ID NO: 795 - RNA, 20 nt, Canis sp.
  source 1..20: mol_type = other RNA
ctttccatccttcatcacac

SEQ ID NO: 796 - RNA, 20 nt, Canis sp.
  source 1..20: mol_type = other RNA
gagaactgtggttcaggtct

SEQ ID NO: 797 - RNA, 20 nt, Canis sp.
  source 1..20: mol_type = other RNA
ggagtaagccatcatttcac

SEQ ID NO: 798 - RNA, 20 nt, Canis sp.
  source 1..20: mol_type = other RNA
gtgaaatgatggcttactcc

SEQ ID NO: 799 - RNA, 20 nt, Canis sp.
  source 1..20: mol_type = other RNA
ttcgggctctccacctcaat

SEQ ID NO: 800 - RNA, 20 nt, Canis sp.
  source 1..20: mol_type = other RNA
agctcatgtggaacaccact

SEQ ID NO: 801 - RNA, 20 nt, Equus caballus
  source 1..20: mol_type = other RNA
aagttgtaatgtactgatcg

SEQ ID NO: 802 - RNA, 21 nt, Equus caballus
  source 1..21: mol_type = other RNA
catcattcatgagtacactgg

SEQ ID NO: 803 - RNA, 20 nt, Equus caballus
  source 1..20: mol_type = other RNA
gggcatcattcagagtacac

SEQ ID NO: 804 - RNA, 20 nt, Equus caballus
  source 1..20: mol_type = other RNA
ggaagttgtaatgtactgat

SEQ ID NO: 805 - RNA, 20 nt, Equus caballus
  source 1..20: mol_type = other RNA
ttgccagtgaaatttgacat

SEQ ID NO: 806 - RNA, 20 nt, Equus caballus
  source 1..20: mol_type = other RNA
gtcatagcttgcatcataga

SEQ ID NO: 807 - RNA, 20 nt, Equus caballus
  source 1..20: mol_type = other RNA
aagctatgacccacttcctg

SEQ ID NO: 808 - RNA, 20 nt, Equus caballus
  source 1..20: mol_type = other RNA
gctgaacttcaaggagagcg

SEQ ID NO: 809 - RNA, 20 nt, Equus caballus
  source 1..20: mol_type = other RNA
gtggtgctggtggcagccaa

SEQ ID NO: 810 - RNA, 20 nt, Equus caballus
  source 1..20: mol_type = other RNA
cttcttcagagtcttcccgt

SEQ ID NO: 811 - RNA, 20 nt, Equus caballus
  source 1..20: mol_type = other RNA
aggacagacctttagcagta

SEQ ID NO: 812 - RNA, 20 nt, Equus caballus
  source 1..20: mol_type = other RNA
agtgtaattcgagacacatc

SEQ ID NO: 813 - RNA, 20 nt, Equus caballus
  source 1..20: mol_type = other RNA
ggacagacctttagcagtac

SEQ ID NO: 814 - RNA, 20 nt, Equus caballus
  source 1..20: mol_type = other RNA
tggtgctggtggcagccaac

SEQ ID NO: 815 - RNA, 20 nt, Equus caballus
  source 1..20: mol_type = other RNA
ctcgaattacactttgattg

SEQ ID NO: 816 - RNA, 20 nt, Equus caballus
  source 1..20: mol_type = other RNA
cctgaagaactgttacaggt

SEQ ID NO: 817 - RNA, 20 nt, Equus caballus
  source 1..20: mol_type = other RNA
gctctccttgaagttcagct

SEQ ID NO: 818 - RNA, 20 nt, Equus caballus
  source 1..20: mol_type = other RNA
gaagttgtaatgtagtgatc

SEQ ID NO: 819 - RNA, 20 nt, Equus caballus
  source 1..20: mol_type = other RNA
cccacttcctgaggactgca

SEQ ID NO: 820 - RNA, 20 nt, Equus caballus
  source 1..20: mol_type = other RNA
ccatgcagtcctcaggaagt

SEQ ID NO: 821 - RNA, 20 nt, Equus caballus
  source 1..20: mol_type = other RNA
gttcagcttggatgtcttag

SEQ ID NO: 822 - RNA, 20 nt, Equus caballus
  source 1..20: mol_type = other RNA
aatggcttccaggtcatcta

SEQ ID NO: 823 - RNA, 20 nt, Equus caballus
  source 1..20: mol_type = other RNA
tcgaattacactttgattga

SEQ ID NO: 824 - RNA, 20 nt, Equus caballus
  source 1..20: mol_type = other RNA
gcacccatgtcaaatttcac

SEQ ID NO: 825 - RNA, 20 nt, Equus caballus
  source 1..20: mol_type = other RNA
aatgtatccatgcagtcctc

SEQ ID NO: 826 - RNA, 20 nt, Equus caballus
  source 1..20: mol_type = other RNA
ggatgtcttagaggtttcag

SEQ ID NO: 827 - RNA, 20 nt, Equus caballus
  source 1..20: mol_type = other RNA
gggtgcttatacatcagaag

SEQ ID NO: 828 - RNA, 20 nt, Equus caballus
  source 1..20: mol_type = other RNA
agatgaacccgtactgctaa

SEQ ID NO: 829 - RNA, 20 nt, Equus caballus
  source 1..20: mol_type = other RNA
tcaggtcttcaaagaggtca

SEQ ID NO: 830 - RNA, 20 nt, Equus caballus
  source 1..20: mol_type = other RNA
gacatccaagctgaacttca

SEQ ID NO: 831 - RNA, 20 nt, Equus caballus
  source 1..20: mol_type = other RNA
gcatgtcgtgtcaatcattg

SEQ ID NO: 832 - RNA, 20 nt, Equus caballus
  source 1..20: mol_type = other RNA
ctcagctccatgggcgatgg

SEQ ID NO: 833 - RNA, 20 nt, Equus caballus
  source 1..20: mol_type = other RNA
aagtcttgttgtaaagttgg

SEQ ID NO: 834 - RNA, 20 nt, Equus caballus
  source 1..20: mol_type = other RNA
ctgcaggctctccacctcaa

SEQ ID NO: 835 - RNA, 20 nt, Equus caballus
  source 1..20: mol_type = other RNA
ggtttgtattctctccattg

SEQ ID NO: 836 - RNA, 20 nt, Equus caballus
  source 1..20: mol_type = other RNA
ctcacctccagctgtagggt

SEQ ID NO: 837 - RNA, 20 nt, Equus caballus
  source 1..20: mol_type = other RNA
attcagtgaactgcagactc

SEQ ID NO: 838 - RNA, 20 nt, Equus caballus
  source 1..20: mol_type = other RNA
gggatttatggtatatgtcc

SEQ ID NO: 839 - RNA, 20 nt, Equus caballus
  source 1..20: mol_type = other RNA
cataaatccctggtgctgtc

SEQ ID NO: 840 - RNA, 20 nt, Equus caballus
  source 1..20: mol_type = other RNA
actcacctccagctctaggg

SEQ ID NO: 841 - RNA, 20 nt, Equus caballus
  source 1..20: mol_type = other RNA
gttggtgggagaattgaagc

SEQ ID NO: 842 - RNA, 20 nt, Equus caballus
  source 1..20: mol_type = other RNA
ccttccaggatgatgacttg

SEQ ID NO: 843 - RNA, 20 nt, Equus caballus
  source 1..20: mol_type = other RNA
acatgcaccggacagcacca

SEQ ID NO: 844 - RNA, 20 nt, Equus caballus
  source 1..20: mol_type = other RNA
ttctgcatgagctttgtgca

SEQ ID NO: 845 - RNA, 20 nt, Equus caballus
  source 1..20: mol_type = other RNA
gaagcccaccctacagctgg

SEQ ID NO: 846 - RNA, 20 nt, Equus caballus
  source 1..20: mol_type = other RNA
ctggatgcccccatcgccca

SEQ ID NO: 847 - RNA, 20 nt, Equus caballus
  source 1..20: mol_type = other RNA
agtcttgttgtaaagttggt

SEQ ID NO: 848 - RNA, 20 nt, Equus caballus
  source 1..20: mol_type = other RNA
ggacatataccataaatccc

SEQ ID NO: 849 - RNA, 21 nt, Equus caballus
  source 1..21: mol_type = other RNA
aagaaacctgtacctgtcttg

SEQ ID NO: 850 - RNA, 20 nt, Equus caballus
  source 1..20: mol_type = other RNA
tgtcttgtgggatgaaagat

SEQ ID NO: 851 - RNA, 20 nt, Equus caballus
  source 1..20: mol_type = other RNA
gatacccgttccctgctcac

SEQ ID NO: 852 - RNA, 20 nt, Equus caballus
  source 1..20: mol_type = other RNA
ggagagaatacaaaccaaca

SEQ ID NO: 853 - RNA, 20 nt, Equus caballus
  source 1..20: mol_type = other RNA
gttcttttccttgaggccca

SEQ ID NO: 854 - RNA, 20 nt, Equus caballus
  source 1..20: mol_type = other RNA
catctttcatcccacaagac

SEQ ID NO: 855 - RNA, 20 nt, Equus caballus
  source 1..20: mol_type = other RNA
gcagtaagtcatcatgtcac

SEQ ID NO: 856 - RNA, 20 nt, Equus caballus
  source 1..20: mol_type = other RNA
tgaaagtcttgttgtaaagt

SEQ ID NO: 857 - RNA, 20 nt, Equus caballus
  source 1..20: mol_type = other RNA
agaacctgtacctgtcttgt

SEQ ID NO: 858 - RNA, 20 nt, Equus caballus
  source 1..20: mol_type = other RNA
ctgtcttgtgggatgaaaga

SEQ ID NO: 859 - RNA, 21 nt, Equus caballus
  source 1..21: mol_type = other RNA
tgggaagcccaccctacacgc

SEQ ID NO: 860 - RNA, 20 nt, Equus caballus
  source 1..20: mol_type = other RNA
ttcactcacctccagctgta

SEQ ID NO: 861 - RNA, 20 nt, Felis catus
  source 1..20: mol_type = other RNA
aagttgggtgctactgatct

SEQ ID NO: 862 - RNA, 20 nt, Felis catus
  source 1..20: mol_type = other RNA
gccagtgaaattggacatgg

SEQ ID NO: 863 - RNA, 20 nt, Felis catus
  source 1..20: mol_type = other RNA
cttccaggtcatcagcagtg

SEQ ID NO: 864 - RNA, 20 nt, Felis catus
  source 1..20: mol_type = other RNA
ttcgaattacactttgactg

SEQ ID NO: 865 - RNA, 20 nt, Felis catus
  source 1..20: mol_type = other RNA
tctgtaatgcagcggctgcg

SEQ ID NO: 866 - RNA, 20 nt, Felis catus
  source 1..20: mol_type = other RNA
gcttaccctcaagaagagtg

SEQ ID NO: 867 - RNA, 20 nt, Felis catus
  source 1..20: mol_type = other RNA
tggtgatggtggcagccaat

SEQ ID NO: 868 - RNA, 20 nt, Felis catus
  source 1..20: mol_type = other RNA
ctcatagcttgcatcataga

SEQ ID NO: 869 - RNA, 20 nt, Felis catus
  source 1..20: mol_type = other RNA
ctgtacagtcctcatgaagt

SEQ ID NO: 870 - RNA, 20 nt, Felis catus
  source 1..20: mol_type = other RNA
ttgccagtgaaatttgacat

SEQ ID NO: 871 - RNA, 20 nt, Felis catus
  source 1..20: mol_type = other RNA
cctgaagaactgttacaggt

SEQ ID NO: 872 - RNA, 20 nt, Felis catus
  source 1..20: mol_type = other RNA
aagctatgacccacttcatg

SEQ ID NO: 873 - RNA, 20 nt, Felis catus
  source 1..20: mol_type = other RNA
ttagaggtttcagaagtact

SEQ ID NO: 874 - RNA, 20 nt, Felis catus
  source 1..20: mol_type = other RNA
ctcttcttgagggtaagctg

SEQ ID NO: 875 - RNA, 20 nt, Felis catus
  source 1..20: mol_type = other RNA
actcttcttgagggtaagct

SEQ ID NO: 876 - RNA, 20 nt, Felis catus
  source 1..20: mol_type = other RNA
gtggtgatggtggcagccaa

SEQ ID NO: 877 - RNA, 20 nt, Felis catus
  source 1..20: mol_type = other RNA
aaagtgtaattcgaaaagca

SEQ ID NO: 878 - RNA, 20 nt, Felis catus
  source 1..20: mol_type = other RNA
cgactgacctttagcaatac

SEQ ID NO: 879 - RNA, 20 nt, Felis catus
  source 1..20: mol_type = other RNA
agtgtaattcgaaaagcagg

SEQ ID NO: 880 - RNA, 20 nt, Felis catus
  source 1..20: mol_type = other RNA
agccgctgcattacagaatc

SEQ ID NO: 881 - RNA, 20 nt, Felis catus
  source 1..20: mol_type = other RNA
atccagattctgtaatgcag

SEQ ID NO: 882 - RNA, 20 nt, Felis catus
  source 1..20: mol_type = other RNA
ggtaagctggggtgtcttag

SEQ ID NO: 883 - RNA, 20 nt, Felis catus
  source 1..20: mol_type = other RNA
gaagttgggtgctactgatc

SEQ ID NO: 884 - RNA, 20 nt, Felis catus
  source 1..20: mol_type = other RNA
caaagtgtaattcgaaaagc

SEQ ID NO: 885 - RNA, 20 nt, Felis catus
  source 1..20: mol_type = other RNA
tgccagtgaaatttgacatg

SEQ ID NO: 886 - RNA, 20 nt, Felis catus
  source 1..20: mol_type = other RNA
gcccccatctcaaatttcac

SEQ ID NO: 887 - RNA, 20 nt, Felis catus
  source 1..20: mol_type = other RNA
tctgtacagtcctcatgaag

SEQ ID NO: 888 - RNA, 20 nt, Felis catus
  source 1..20: mol_type = other RNA
catcaccacactcttcttga

SEQ ID NO: 889 - RNA, 20 nt, Felis catus
  source 1..20: mol_type = other RNA
attcctcactgctgatgacc

SEQ ID NO: 890 - RNA, 20 nt, Felis catus
  source 1..20: mol_type = other RNA
ttttcactgctatagaagtt

SEQ ID NO: 891 - RNA, 20 nt, Felis catus
  source 1..20: mol_type = other RNA
gagtcttaggcatgccgtgt

SEQ ID NO: 892 - RNA, 20 nt, Felis catus
  source 1..20: mol_type = other RNA
catggccacaatgaccgaca

SEQ ID NO: 893 - RNA, 20 nt, Felis catus
  source 1..20: mol_type = other RNA
tgatgcggccatacaatcac

SEQ ID NO: 894 - RNA, 20 nt, Felis catus
  source 1..20: mol_type = other RNA
acccatcatctgtgacacgt

SEQ ID NO: 895 - RNA, 20 nt, Felis catus
  source 1..20: mol_type = other RNA
ccgcatgagctttgtgcacg

SEQ ID NO: 896 - RNA, 20 nt, Felis catus
  source 1..20: mol_type = other RNA
ggttcatattctgtccattg

SEQ ID NO: 897 - RNA, 20 nt, Felis catus
  source 1..20: mol_type = other RNA
catgagctttgtgcacgggg

SEQ ID NO: 898 - RNA, 20 nt, Felis catus
  source 1..20: mol_type = other RNA
cgtgtagtcctgtgattgta

SEQ ID NO: 899 - RNA, 20 nt, Felis catus
  source 1..20: mol_type = other RNA
ttccgcatgagctttgtgca

SEQ ID NO: 900 - RNA, 20 nt, Felis catus
  source 1..20: mol_type = other RNA
gcatgccgtgtcggtcattg

SEQ ID NO: 901 - RNA, 20 nt, Felis catus
  source 1..20: mol_type = other RNA
tgtctggctcatacgagctt

SEQ ID NO: 902 - RNA, 20 nt, Felis catus
  source 1..20: mol_type = other RNA
ggtactcacctccagctgta

SEQ ID NO: 903 - RNA, 20 nt, Felis catus
  source 1..20: mol_type = other RNA
catggccacttcagaaacct

SEQ ID NO: 904 - RNA, 20 nt, Felis catus
  source 1..20: mol_type = other RNA
aacccatcatctgtgacacg

SEQ ID NO: 905 - RNA, 20 nt, Felis catus
  source 1..20: mol_type = other RNA
gtctggctcatacgagcttc

SEQ ID NO: 906 - RNA, 20 nt, Felis catus
  source 1..20: mol_type = other RNA
cttcgggctctccacctcaa

SEQ ID NO: 907 - RNA, 20 nt, Felis catus
  source 1..20: mol_type = other RNA
tccgcatgagctttgtgcac

SEQ ID NO: 908 - RNA, 20 nt, Felis catus
  source 1..20: mol_type = other RNA
actcacctccagctgtaggg

SEQ ID NO: 909 - RNA, 20 nt, Felis catus
  source 1..20: mol_type = other RNA
cgtatgagccagacagcacc

SEQ ID NO: 910 - RNA, 20 nt, Felis catus
  source 1..20: mol_type = other RNA
ggacagaatatgaaccaaca

SEQ ID NO: 911 - RNA, 20 nt, Felis catus
  source 1..20: mol_type = other RNA
aacgtccccagttaccttgt

SEQ ID NO: 912 - RNA, 20 nt, Felis catus
  source 1..20: mol_type = other RNA
gaaacccaccctacagctgg

SEQ ID NO: 913 - RNA, 20 nt, Felis catus
  source 1..20: mol_type = other RNA
ctcacctccagctgtagggt

SEQ ID NO: 914 - RNA, 20 nt, Felis catus
  source 1..20: mol_type = other RNA
gtagtagaccatcatttcac

SEQ ID NO: 915 - RNA, 20 nt, Felis catus
  source 1..20: mol_type = other RNA
gtctcaccttcatcttttcg

SEQ ID NO: 916 - RNA, 20 nt, Felis catus
  source 1..20: mol_type = other RNA
cctcatctcccagaaaactg

SEQ ID NO: 917 - RNA, 20 nt, Felis catus
  source 1..20: mol_type = other RNA
agcccgacaacaagagtctt

SEQ ID NO: 918 - RNA, 20 nt, Felis catus
  source 1..20: mol_type = other RNA
atctgtgacacgtgggatga

SEQ ID NO: 919 - RNA, 20 nt, Felis catus
  source 1..20: mol_type = other RNA
tgtcctgtgtgatgaaagat

SEQ ID NO: 920 - RNA, 20 nt, Felis catus
  source 1..20: mol_type = other RNA
actcttgttgtcgggctggt

SEQ ID NO: 921 - RNA, 20 nt, Synthetic construct
  source 1..20: mol_type = other RNA
acttttgtttcagagctggt

SEQ ID NO: 922 - RNA, 20 nt, Synthetic construct
  source 1..20: mol_type = other RNA
cctcatgctacagagctggt

SEQ ID NO: 923 - RNA, 20 nt, Synthetic construct
  source 1..20: mol_type = other RNA
gtgcttgttacagagctggt

SEQ ID NO: 924 - RNA, 20 nt, Homo sapiens
  source 1..20: mol_type = other RNA
gtgcagttcagcgatcgtac

SEQ ID NO: 925 - RNA, 20 nt, Homo sapiens
  source 1..20: mol_type = other RNA
tgatggccctaaacagatga

SEQ ID NO: 926 - RNA, 20 nt, Homo sapiens
  source 1..20: mol_type = other RNA
acctatcttcttcgacacat

SEQ ID NO: 927 - RNA, 20 nt, Homo sapiens
  source 1..20: mol_type = other RNA
cttcgacacatgggacaacg

SEQ ID NO: 928 - RNA, 20 nt, Homo sapiens
  source 1..20: mol_type = other RNA
catgggcacaacaactgacg

SEQ ID NO: 929 - RNA, 20 nt, Homo sapiens
  source 1..20: mol_type = other RNA
aggtcctggaaggagcactg

SEQ ID NO: 930 - RNA, 20 nt, Canis sp.
  source 1..20: mol_type = other RNA
tgaccatctctctctgaatc

SEQ ID NO: 931 - RNA, 20 nt, Canis sp.
  source 1..20: mol_type = other RNA
agtatagttcgacaaacagg

SEQ ID NO: 932 - RNA, 20 nt, Canis sp.
  source 1..20: mol_type = other RNA
tctgtaatgcagcagtcatg

SEQ ID NO: 933 - RNA, 20 nt, Canis sp.
  source 1..20: mol_type = other RNA
cgacggccctggaaatgtga

SEQ ID NO: 934 - RNA, 20 nt, Canis sp.
  source 1..20: mol_type = other RNA
cacatccctccttaccttca

SEQ ID NO: 935 - RNA, 20 nt, Canis sp.
  source 1..20: mol_type = other RNA
taaataccagcccaccttca

SEQ ID NO: 936 - RNA, 20 nt, Canis sp.
  source 1..20: mol_type = other RNA
gtggacccagcttaccttca

SEQ ID NO: 937 - RNA, 20 nt, Canis sp.
  source 1..20: mol_type = other RNA
acttttgtttcagagctggt

SEQ ID NO: 938 - RNA, 20 nt, Canis sp.
  source 1..20: mol_type = other RNA
cctcacgctacagagctggt

SEQ ID NO: 939 - RNA, 20 nt, Homo sapiens
  source 1..20: mol_type = other RNA
caacatcttcttcgacacat

SEQ ID NO: 940 - RNA, 20 nt, Canis sp.
  source 1..20: mol_type = other RNA
tgatggccctggaaatgtga

SEQ ID NO: 941 - protein, 263 aa, Synthetic construct
MAKVPDLFEDLKNCYSENEEYSSEIDHLSLNQKSFYDMSCDPLHEDCMSLSTSEISKTSQLTFKENVVVVAANGKILKKRRLSLSQFITDDDLEDIANDTEEVIMKPRSVAYNFHNNEKYNYIRIIKSQFILNDNLNQSIVRQTGGNYLMTAALQNLDDAVKFDMGAYTSEDSKLPVTLRISKTRLFVSAQNEDEPVLLKEMPETPKTIRDDETNLLFFWERHGSKHYSVAQPKLPIATKRKLVHMARGQPSITDFRLLETQP

SEQ ID NO: 942 - protein, 91 aa, Synthetic construct
MAKVPDLPEDLKNCYSENEEYSSEIDHLSLNQKSFYDMSCDDPLHEDCMSLSTSEISKTSQLTPQGKCGSGGSQWEDSKEETVEFKSIHHR

SEQ ID NO: 943 - RNA, 20 nt, Synthetic construct
  source 1..20: mol_type = other RNA
tgaccatctctctctgaatc

SEQ ID NO: 944 - RNA, 20 nt, Synthetic construct
  source 1..20: mol_type = other RNA
acatgcagtcctcatgaagt

SEQ ID NO: 945 - RNA, 20 nt, Synthetic construct
  source 1..20: mol_type = other RNA
gacatcccagcttaccttca

SEQ ID NO: 946 - RNA, 20 nt, Synthetic construct
  source 1..20: mol_type = other RNA
gtcgaactatactttgattg

SEQ ID NO: 947 - RNA, 20 nt, Synthetic construct
  source 1..20: mol_type = other RNA
agtatagttcgacaaacagg

SEQ ID NO: 948 - RNA, 20 nt, Synthetic construct
  source 1..20: mol_type = other RNA
aagttgtacgctactgatct

SEQ ID NO: 949 - RNA, 20 nt, Synthetic construct
  source 1..20: mol_type = other RNA
caaagtatagttcgacaaac

SEQ ID NO: 950 - RNA, 20 nt, Synthetic construct
  source 1..20: mol_type = other RNA
tctgtaatgcagcagtcatg

SEQ ID NO: 951 - RNA, 20 nt, Synthetic construct
  source 1..20: mol_type = other RNA
cttgcagtccaccgattgca

SEQ ID NO: 952 - RNA, 20 nt, Synthetic construct
  source 1..20: mol_type = other RNA
ttcgggctctccacctcaat

SEQ ID NO: 953 - RNA, 20 nt, Synthetic construct
  source 1..20: mol_type = other RNA
tgatgcagccatgcaatcgg

SEQ ID NO: 954 - RNA, 20 nt, Synthetic construct
  source 1..20: mol_type = other RNA
tcgggctctccacctcaatg

SEQ ID NO: 955 - RNA, 21 nt, Synthetic construct
  source 1..21: mol_type = other RNA
gagtgacgcagcccatgcaat

SEQ ID NO: 956 - RNA, 20 nt, Synthetic construct
  source 1..20: mol_type = other RNA
tcagactcttgttacagagc

SEQ ID NO: 957 - RNA, 20 nt, Synthetic construct
  source 1..20: mol_type = other RNA
actcttgttacagagctggt

SEQ ID NO: 958 - RNA, 20 nt, Synthetic construct
  source 1..20: mol_type = other RNA
tgatggccctggaaatgtga

SEQ ID NO: 959 - DNA, 59 bp, Mus musculus
  source 1..59: mol_type = other DNA
atggccaaagttcctgacttgtttgaagacctaaagaactgttacagtgaaaacgaaga

SEQ ID NO: 960 - DNA, 60 bp, Homo sapiens
  source 1..60: mol_type = other DNA
atggccaaagttccagacatgtttgaagacctgaagaactgttacagtgaaaatgaagaa

SEQ ID NO: 961 - DNA, 60 bp, Equus caballus
  source 1..60: mol_type = other DNA
atggcgaaagtccctgacctctttgaagacctgaagaactgttacagtgaaaatgaagac

SEQ ID NO: 962 - DNA, 60 bp, Felis catus
  source 1..60: mol_type = other DNA
atggccaaagttcctgacctctttgaagacctgaagaactgttacagtgaaaatgaagaa

SEQ ID NO: 963 - DNA, 60 bp, Canis sp.; identical to SEQ ID NO: 962
  source 1..60: mol_type = other DNA

SEQ ID NO: 964 - DNA, 60 bp, Mus musculus
  source 1..60: mol_type = other DNA
tacagttctgccattgaccatctctctctgaatcagaaatccttctatgatgcaagctat

SEQ ID NO: 965 - DNA, 60 bp, Homo sapiens
  source 1..60: mol_type = other DNA
gacagttcctccattgatcatctgtctctgaatcagaaatccttctatcatgtaagctat

SEQ ID NO: 966 - DNA, 60 bp, Equus caballus
  source 1..60: mol_type = other DNA
tacagttctgaaattgaccatctctctctgactcagaaatccttctatgatgcaagctat

SEQ ID NO: 967 - DNA, 60 bp, Felis catus
  source 1..60: mol_type = other DNA
tacagttctgaaattgaccatctcactctgaatcagaaatccttctatgatgcaagctat

SEQ ID NO: 968 - DNA, 60 bp, Canis sp.
  source 1..60: mol_type = other DNA
tacagttctgaaattgaccatctctctctgaatcagaaatccttctatgatatgagctgt

SEQ ID NO: 969 - DNA, 60 bp, Mus musculus
  source 1..60: mol_type = other DNA
ggctcacttcatgagacttgcacagatcagtttgtatctctgagaacctctgaaacgtca

SEQ ID NO: 970 - DNA, 60 bp, Homo sapiens
  source 1..60: mol_type = other DNA
ggcccactccatgaaggctgcatggatcaatctgtgtctctgagtatctctgaaacctct

SEQ ID NO: 971 - DNA, 60 bp, Equus caballus
  source 1..60: mol_type = other DNA
gacccacttcctgaggactgcatggatacatttatgtctctgagcacctctgaaacctct

SEQ ID NO: 972 - DNA, 60 bp, Felis catus
  source 1..60: mol_type = other DNA
gacccacttcatgaggactgtacagataaattcatgtctccgagtacttctgaaacctct

SEQ ID NO: 973 - DNA, 48 bp, Canis sp.
  source 1..48: mol_type = other DNA
gacccacttcatgaggactgcatgtctctgagtacctctgaaatctca

SEQ ID NO: 974 - DNA, 60 bp, Mus musculus
  source 1..60: mol_type = other DNA
aagatgtccaacttcaccttcaaggagagccgggtgacagtatcagcaacgtcaagcaac

SEQ ID NO: 975 - DNA, 51 bp, Homo sapiens
  source 1..51: mol_type = other DNA
aaaacatccaagcttaccttcaaggagagcatggtggtagtagcaaccaac

SEQ ID NO: 976 - DNA, 51 bp, Equus caballus
  source 1..51: mol_type = other DNA
aagacatccaagctgaacttcaaggagagcgtggtgctggtggcagccaac

SEQ ID NO: 977 - DNA, 51 bp, Felis catus
  source 1..51: mol_type = other DNA
aagacaccccagcttaccctcaagaagagtgtggtgatggtggcagccaat

SEQ ID NO: 978 - DNA, 51 bp, Canis sp.
  source 1..51: mol_type = other DNA
aagacatcccagcttaccttcaaggaaaatgtggtagtggtggcagccaat

SEQ ID NO: 979 - DNA, 60 bp, Mus musculus
  source 1..60: mol_type = other DNA
gggaagattctgaagaagagacggctgagtttcagtgagaccttcactgaagatgacctg

SEQ ID NO: 980 - DNA, 60 bp, Homo sapiens
  source 1..60: mol_type = other DNA
gggaaggttctgaagaagagacggttgagtttaagccaatccatcactgatgatgacctg

SEQ ID NO: 981 - DNA, 60 bp, Equus caballus
  source 1..60: mol_type = other DNA
gggaagactctgaagaagagacggttgagtttaaatcagttcatcaccaatgatgacctg

SEQ ID NO: 982 - DNA, 60 bp, Felis catus
  source 1..60: mol_type = other DNA
gggaagattctgaagaagagacggttgagtttaaatcaattcctcactgctgatgacctg

SEQ ID NO: 983 - DNA, 60 bp, Canis sp.
  source 1..60: mol_type = other DNA
gggaagattctaaagaagagacggttgagtttaagtcaattcatcaccgatgatgacctg

SEQ ID NO: 984 - DNA, 57 bp, Mus musculus
  source 1..57: mol_type = other DNA
cagtccataacccatgatctggaagagaccatccaacccagatcagcaccttacacc

SEQ ID NO: 985 - DNA, 60 bp, Homo sapiens
  source 1..60: mol_type = other DNA
gaggccatcgccaatgactcagaggaagaaatcatcaagcctaggtcagcaccttttagc

SEQ ID NO: 986 - DNA, 60 bp, Equus caballus
  source 1..60: mol_type = other DNA
gaagccattgccaatgatccagaagaaggaatcatcaagccccgatcagtacattacaac

SEQ ID NO: 987 - DNA, 60 bp, Felis catus
  source 1..60: mol_type = other DNA
gaagccattgccaacgaagtagaagaagaaatcatgaagcccagatcagtagcacccaac

SEQ ID NO: 988 - DNA, 60 bp, Canis sp.
  source 1..60: mol_type = other DNA
gaagacattgccaatgacacagaagaagtaatcatgaagcccagatcagtagcatacaac

SEQ ID NO: 989 - DNA, 60 bp, Mus musculus
  source 1..60: mol_type = other DNA
taccagagtgatttgagatacaaactgatgaagctcgtcaggcagaagtttgtcatgaat

SEQ ID NO: 990 - DNA, 60 bp, Homo sapiens
  source 1..60: mol_type = other DNA
ttcctgagcaatgtgaaatacaactttatgaggatcatcaaatacgaattcatcctgaat

SEQ ID NO: 991 - DNA, 60 bp, Equus caballus
  source 1..60: mol_type = other DNA
ttccagagcaatacaaaatacaactttatgaggatcgtcaaccaccagtgtactctgaat

SEQ ID NO: 992 - DNA, 60 bp, Felis catus
  source 1..60: mol_type = other DNA
ttctatagcagtgaaaaatacaactatcagaagatcatcaaatcccaattcatcctgaat

SEQ ID NO: 993 - DNA, 60 bp, Canis sp.
  source 1..60: mol_type = other DNA
ttccataacaatgaaaaatacaactatataaggatcatcaaatcccaattcatcctgaat

SEQ ID NO: 994 - DNA, 60 bp, Mus musculus
  source 1..60: mol_type = other DNA
gattccctcaaccaaactatatatcaggatgtggacaaacactatctcagcaccacttgg

SEQ ID NO: 995 - DNA, 57 bp, Homo sapiens
  source 1..57: mol_type = other DNA
gacgccctcaatcaaagtataattcgagccaatgatcagtacctcacggctgctgca

SEQ ID NO: 996 - DNA, 60 bp, Equus caballus
  source 1..60: mol_type = other DNA
gatgccctcaatcaaagtgtaattcgagacacatcaggtcaatatcttgcgactgctgca

SEQ ID NO: 997 - DNA, 60 bp, Felis catus
  source 1..60: mol_type = other DNA
gacaacctcagtcaaagtgtaattcgaaaagcagggggaaaatacctcgcagccgctgca

SEQ ID NO: 998 - DNA, 60 bp, Canis sp.
  source 1..60: mol_type = other DNA
gacaacctcaatcaaagtatagttcgacaaacaggaggaaattacctcatgactgctgca

SEQ ID NO: 999 - DNA, 60 bp, Mus musculus
  source 1..60: mol_type = other DNA
ttaaatgacctgcaacaggaagtaaaatttgacatgtatgcctactcgtcgggaggagac

SEQ ID NO: 1000 - DNA, 60 bp, Homo sapiens
  source 1..60: mol_type = other DNA
ttacataatctggatgaagcagtgaaatttgacatgggtgcttataagtcatcaaaggat

SEQ ID NO: 1001 - DNA, 57 bp, Equus caballus
  source 1..57: mol_type = other DNA
ttaaataatctggacgacgcagtgaaatttgacatgggtgcttatacatcagaagag

SEQ ID NO: 1002 - DNA, 57 bp, Felis catus
  source 1..57: mol_type = other DNA
ttacagaatctggatgatgcagtgaaatttgacatgggggcttatacatcaaaagaa

SEQ ID NO: 1003 - DNA, 54 bp, Canis sp.
  source 1..54: mol_type = other DNA
ttacagaatttggatgatgcagtgaagtttgacatgggagcttacacatcagaa

SEQ ID NO: 1004 - DNA, 60 bp, Mus musculus
  source 1..60: mol_type = other DNA
gactctaaatatcctgttactctaaaaatctcagattcacaactgttcgtgagcgctcaa

SEQ ID NO: 1005 - DNA, 60 bp, Homo sapiens
  source 1..60: mol_type = other DNA
gatgctaaaattaccgtgattctaagaatctcaaaaactcaattgtatgtgactgcccaa

SEQ ID NO: 1006 - DNA, 60 bp, Equus caballus
  source 1..60: mol_type = other DNA
gattctcaacttcctgtgactctaagaatctcaaaaactcgactgtttgtgagtgcccaa

SEQ ID NO: 1007 - DNA, 60 bp, Felis catus
  source 1..60: mol_type = other DNA
gattctaaacttcctgtgactctcagaatctcaaaaactcgactgtttgtgagtgcccaa

SEQ ID NO: 1008 - DNA, 60 bp, Canis sp.
  source 1..60: mol_type = other DNA
gattctaaacttcctgtgactctaagaatatcaaaaactcgactctttgtgagtgcccaa

SEQ ID NO: 1009 - DNA, 60 bp, Mus musculus
  source 1..60: mol_type = other DNA
ggagaagaccagcccgtgttgctgaaggagttgccagaaacaccaaaactcatcacaggt

SEQ ID NO: 1010 - DNA, 60 bp, Homo sapiens
  source 1..60: mol_type = other DNA
gatgaagaccaaccagtgctgctgaaggagatgcctgagatacccaaaaccatcacaggt

SEQ ID NO: 1011 - DNA, 57 bp, Equus caballus
  source 1..57: mol_type = other DNA
aatgaagatgaacccgtactgctaaaggagatgcctgacacacccaaaactatcaaa

SEQ ID NO: 1012 - DNA, 58 bp, Felis catus
  source 1..58: mol_type = other DNA
aatgaagatgagcctgtattgctaaaggagatgcctgagacacccaaaaccatcagag

SEQ ID NO: 1013 - DNA, 58 bp, Canis sp.
  source 1..58: mol_type = other DNA
aatgaagatgaacctgtattgctaaaggagatgcctgagacacccaaaactatcagag

SEQ ID NO: 1014 - DNA, 60 bp, Mus musculus
  source 1..60: mol_type = other DNA
agtgagaccgacctcattttcttctggaaaagtatcaactctaagaactacttcacatca

SEQ ID NO: 1015 - DNA, 60 bp, Homo sapiens
  source 1..60: mol_type = other DNA
agtgagaccaacctcctcttcttctgggaaactcacggcactaagaactatttcacatca

SEQ ID NO: 1016 - DNA, 59 bp, Equus caballus
  source 1..59: mol_type = other DNA
atgagaccaacctcctcttcttctgggaacgtcacggctctaagaactacttcaaatcg

SEQ ID NO: 1017 - DNA, 59 bp, Felis catus
  source 1..59: mol_type = other DNA
atgagaccaaccttctcttcttctgggaacgtcatggcagtaagaactacttcaaatca

SEQ ID NO: 1018 - DNA, 59 bp, Canis sp.
  source 1..59: mol_type = other DNA
atgagacaaaccttcttttcttttgggagcgtcatggcagtaagcactacttcaaatca

SEQ ID NO: 1019 - DNA, 60 bp, Mus musculus
  source 1..60: mol_type = other DNA
gctgcttatccagagctgtttattgccaccaaagaacaaagtcgggtgcacctggcacgg

SEQ ID NO: 1020 - DNA, 60 bp, Homo sapiens
  source 1..60: mol_type = other DNA
gttgcccatccaaacttgtttattgccacaaagcaagactactgggtgtgcttggcaggg

SEQ ID NO: 1021 - DNA, 60 bp, Equus caballus
  source 1..60: mol_type = other DNA
gttgcccatccaaagttgtttattgccacaaagcagggaaaactggtgcacatggcaagg

SEQ ID NO: 1022 - DNA, 60 bp, Felis catus
  source 1..60: mol_type = other DNA
gttgcccatccaaagttgttcattgccacacaggaagaacaactggtgcacatggcaaga

SEQ ID NO: 1023 - DNA, 60 bp, Canis sp.
  source 1..60: mol_type = other DNA
gttgcccagcccaagttgttcattgccacacaggaacgaaaactggtgcacatggcaaga

SEQ ID NO: 1024 - DNA, 60 bp, Canis sp.
  source 1..60: mol_type = other DNA
atgtttcctctgcacccaaaggaaggttctttcatgcattcactcattcatttaaagatt

SEQ ID NO: 1025 - DNA, 59 bp, Canis sp.
  source 1..59: mol_type = other DNA
gtttctttacatgcctgccatgtaccaggtgctaagggaaactcacatatgaaggcagc

SEQ ID NO: 1026 - DNA, 39 bp, Felis catus
  source 1..39: mol_type = other DNA
atgagattgaaattggcagagagcagatctctcgaggca

SEQ ID NO: 1027 - DNA, 60 bp, Canis sp.
  source 1..60: mol_type = other DNA
cctgggggagactggcctggcaggaatcatttattttctcctctttacgcaggtttctaa

SEQ ID NO: 1028 - DNA, 59 bp, Felis catus
  source 1..59: mol_type = other DNA
cagcagcccactccgggattctattctctccagtcagtcttcattactcaggtttctga

SEQ ID NO: 1029 - DNA, 48 bp, Mus musculus
  source 1..48: mol_type = other DNA
ggcaactgttcctgaactcaactgtgaaatgccaccttttgacagtga

SEQ ID NO: 1030 - DNA, 53 bp, Homo sapiens
  source 1..53: mol_type = other DNA
atggcagaagtacctgagctcgccagtgaaatgatggcttattacagtggcaa

SEQ ID NO: 1031 - DNA, 60 bp, Canis sp.
  source 1..60: mol_type = other DNA
agcagccatggcagcagtacccgaactcaccagtgaaatgatggcttactccagtaacaa

SEQ ID NO: 1032 - DNA, 57 bp, Felis catus
  source 1..57: mol_type = other DNA
agtggccatggcagcagtacctgaactcaccagtgaaatgatggcttactacagtga

SEQ ID NO: 1033 - DNA, 50 bp, Equus caballus
  source 1..50: mol_type = other DNA
atggcagcagtacccgacaccagtgacatgatgacttactgcagcggcaa

SEQ ID NO: 1034 - DNA, 60 bp, Mus musculus
  source 1..60: mol_type = other DNA
tgagaatgacctgttctttgaagttgacggaccccaaaagatgaagggctgcttccaaac

SEQ ID NO: 1035 - DNA, 60 bp, Homo sapiens
  source 1..60: mol_type = other DNA
tgaggatgacttgttctttgaagctgatggccctaaacagatgaagtgctccttccagga

SEQ ID NO: 1036 - DNA, 60 bp, Canis sp.
  source 1..60: mol_type = other DNA
tgagaatgacctattctttgaagctgatggccctggaaatgtgaagtgctgctgccaaga

SEQ ID NO: 1037 - DNA, 60 bp, Felis catus
  source 1..60: mol_type = other DNA
tgagaatgacctgttctttgaggctgatggccccgaaaagatgaagggcagcctccaaaa

SEQ ID NO: 1038 - DNA, 60 bp, Equus caballus
  source 1..60: mol_type = other DNA
tgagaatgacctgttctttgaggaggatggcccaaaacagatgaagggcagcttccaaga

SEQ ID NO: 1039 - DNA, 57 bp, Mus musculus
  source 1..57: mol_type = other DNA
ctttgacctgggctgtcctgatgagagcatccagcttcaaatctcgcagcagcacat

SEQ ID NO: 1040 - DNA, 57 bp, Homo sapiens
  source 1..57: mol_type = other DNA
cctggacctctgccctctggatggcggcatccagctacgaatctccgaccaccacta

SEQ ID NO: 1041 - DNA, 60 bp, Canis sp.
  source 1..60: mol_type = other DNA
cctgaaccacagttctctggtagatgagggcatccagttgcaagtctcccaccagctctg

SEQ ID NO: 1042 - DNA, 60 bp, Felis catus
  source 1..60: mol_type = other DNA
cctgagccacagttttctgggagatgagggcatccagttgcaaatctcccaccagcccga

SEQ ID NO: 1043 - DNA, 60 bp, Equus caballus
  source 1..60: mol_type = other DNA
cctggacctcagctccatgggcgatgggggcatccagcttcaattctcccaccaacttta

SEQ ID NO: 1044 - DNA, 60 bp, Mus musculus
  source 1..60: mol_type = other DNA
caacaagagcttcaggcaggcagtatcactcattgtggctgtggagaagctgtggcagct

SEQ ID NO: 1045 - DNA, 60 bp, Homo sapiens
  source 1..60: mol_type = other DNA
cagcaagggcttcaggcaggccgcgtcagttgttgtggccatggacaagctgaggaagat

SEQ ID NO: 1046 - DNA, 58 bp, Canis sp.
  source 1..58: mol_type = other DNA
taacaagagtctgaggcatttcgtgtcagtcattgtagctttggagaagctgaagaag

SEQ ID NO: 1047 - DNA, 60 bp, Felis catus
  source 1..60: mol_type = other DNA
caacaagagtcttaggcatgccgtgtcggtcattgtggccatggagaaactgaagaagat

SEQ ID NO: 1048 - DNA, 60 bp, Equus caballus
  source 1..60: mol_type = other DNA
caacaagactttcaagcatgtcgtgtcaatcattgtggctatggagaagctgaagaagat

SEQ ID NO: 1049 - DNA, 60 bp, Mus musculus
  source 1..60: mol_type = other DNA
acctgtgtctttcccgtggaccttccaggatgaggacatgagcaccttcttttccttcat

SEQ ID NO: 1050 - DNA, 60 bp, Homo sapiens
  source 1..60: mol_type = other DNA
gctggttccctgcccacagaccttccaggagaatgacctgagcaccttctttcccttcat

SEQ ID NO: 1051 - DNA, 50 bp, Canis sp.
  source 1..50: mol_type = other DNA
tgcccacaggtcctccaggaggatgacctgaagagcatcttttgctacat

SEQ ID NO: 1052 - DNA, 60 bp, Felis catus
  source 1..60: mol_type = other DNA
atcttttccctgctcacaacccctccaggatgaggacctgaagagcctcttttgctgcat

SEQ ID NO: 1053 - DNA, 60 bp, Equus caballus
  source 1..60: mol_type = other DNA
acccgttccctgctcacaggccttccaggatgatgacttgaggagcctcttttctgtcat

SEQ ID NO: 1054 - DNA, 60 bp, Mus musculus
  source 1..60: mol_type = other DNA
ctttgaagaagagcccatcctctgtgactcatgggatgatgatgataacctgctggtgtg

SEQ ID NO: 1055 - DNA, 54 bp, Homo sapiens
  source 1..54: mol_type = other DNA
ctttgaagaagaacctatcttcttcgacacatgggataacgaggcttatgtgca

SEQ ID NO: 1056 - DNA, 51 bp, Canis sp.
  source 1..51: mol_type = other DNA
ctttgaagaagaacctatcatctgcaaaacagatgcggataattttatgag

SEQ ID NO: 1057 - DNA, 51 bp, Felis catus
  source 1..51: mol_type = other DNA
ctttgaagaagaacccatcatctgtgacacgtgggatgacggttttgtgtg

SEQ ID NO: 1058 - DNA, 51 bp, Equus caballus
  source 1..51: mol_type = other DNA
ctttgaagaagaacccatcatctgtgacaactgggatgatgattatgtgtg

SEQ ID NO: 1059 - DNA, 60 bp, Mus musculus
  source 1..60: mol_type = other DNA
tgacgttcccattagacaactgcactacaggctccgagatgaacaacaaaaaagcctcgt

SEQ ID NO: 1060 - DNA, 60 bp, Homo sapiens
  source 1..60: mol_type = other DNA
cgatgcacctgtacgatcactgaactgcacgctccgggactcacagcaaaaaagcttggt

SEQ ID NO: 1061 - DNA, 60 bp, Canis sp.
  source 1..60: mol_type = other DNA
tgatgcagccatgcaatcggtggactgcaagttacaggacataagccacaaatacctggt

SEQ ID NO: 1062 - DNA, 60 bp, Felis catus
  source 1..60: mol_type = other DNA
tgatgcggccatacaatcacaggactacacgttccgagacataagccaaaagagcctggt

SEQ ID NO: 1063 - DNA, 60 bp, Equus caballus
  source 1..60: mol_type = other DNA
tgatgcagctgtgcattcagtgaactgcagactccgggacatataccataaatccctggt

SEQ ID NO: 1064 - DNA, 60 bp, Mus musculus
  source 1..60: mol_type = other DNA
gctgtcggacccatatgagctgaaagctctccacctcaatggacagaatatcaaccaaca

SEQ ID NO: 1065 - DNA, 60 bp, Homo sapiens
  source 1..60: mol_type = other DNA
gatgtctggtccatatgaactgaaagctctccacctccagggacaggatatggagcaaca

SEQ ID NO: 1066 - DNA, 60 bp, Canis sp.
  source 1..60: mol_type = other DNA
gctgtctaactcatatgagcttcgggctctccacctcaatggggaaaatgtgaacaaaca

SEQ ID NO: 1067 - DNA, 60 bp, Felis catus
  source 1..60: mol_type = other DNA
gctgtctggctcatacgagcttcgggctctccacctcaatggacagaatatgaaccaaca

SEQ ID NO: 1068 - DNA, 60 bp, Equus caballus
  source 1..60: mol_type = other DNA
gctgtccggtgcatgtgagctgcaggctgtccacctcaatggagagaatacaaaccaaca

SEQ ID NO: 1069 - DNA, 60 bp, Mus musculus
  source 1..60: mol_type = other DNA
agtgatattctccatgagctttgtacaaggagaaccaagcaacgacaaaatacctgtggc

SEQ ID NO: 1070 - DNA, 60 bp, Homo sapiens
  source 1..60: mol_type = other DNA
agtggtgttctccatgtcctttgtacaaggagaagaaagtaatgacaaaatacctgtggc

SEQ ID NO: 1071 - DNA, 60 bp, Canis sp.
  source 1..60: mol_type = other DNA
agtggtgttccacatgagctttgtgcacggggatgaaagtaataacaagatacctgtggt

SEQ ID NO: 1072 - DNA, 60 bp, Felis catus
  source 1..60: mol_type = other DNA
agtggtgttccgcatgagctttgtgcacggggaggaaaatagtaagaagataccagtagt

SEQ ID NO: 1073 - DNA, 60 bp, Equus caballus
  source 1..60: mol_type = other DNA
agtggtgttctgcatgagctttgtgcaaggagaagaagagactgacaagatacctgtggc

SEQ ID NO: 1074 - DNA, 60 bp, Mus musculus
  source 1..60: mol_type = other DNA
cttgggcctcaaaggaaagaatctatacctgtcctgtgtaatgaaagacggcacacccac

SEQ ID NO: 1075 - DNA, 60 bp, Homo sapiens
  source 1..60: mol_type = other DNA
cttgggcctcaaggaaaagaatctgtacctgtcctgcgtgttgaaagatgataagcccac

SEQ ID NO: 1076 - DNA, 60 bp, Canis sp.
  source 1..60: mol_type = other DNA
cttgggcatcaaacaaaagaatctgtacctgtcctgtgtgatgaaggatggaaagcccac

SEQ ID NO: 1077 - DNA, 60 bp, Felis catus
  source 1..60: mol_type = other DNA
gttgtgcatcaagaaaaataacctgtacctgtcctgtgtgatgaaagatgggaaacccac

SEQ ID NO: 1078 - DNA, 60 bp, Equus caballus
  source 1..60: mol_type = other DNA
cttgggcctcaaggaaaagaacctgtacctgtcttgtgggatgaaagatgggaagcccac

SEQ ID NO: 1079 - DNA, 60 bp, Mus musculus
  source 1..60: mol_type = other DNA
cctgcagctggagagtgtggatcccaagcaatacccaaagaagaagatggaaaaacggtt

SEQ ID NO: 1080 - DNA, 60 bp, Homo sapiens
  source 1..60: mol_type = other DNA
tctacagctggagagtgtagatcccaaaaattacccaaagaagaagatggaaaagcgatt

SEQ ID NO: 1081 - DNA, 60 bp, Canis sp.
  source 1..60: mol_type = other DNA
cctacagctagagaaggtagaccccaaagtctacccaaagaggaagatggaaaagcgatt

SEQ ID NO: 1082 - DNA, 60 bp, Felis catus
  source 1..60: mol_type = other DNA
cctacagctggagatgttagaccccaaagtttacccaaagaagaagatggaaaagagatt

SEQ ID NO: 1083 - DNA, 60 bp, Equus caballus
  source 1..60: mol_type = other DNA
cctacagctggagacagtagaccccaatacttacccaaagaggaaaatggaaaagcgatt

SEQ ID NO: 1084 - DNA, 60 bp, Mus musculus
  source 1..60: mol_type = other DNA
tgtcttcaacaagatagaagtcaagagcaaagtggagtttgagtctgcagagttccccaa

SEQ ID NO: 1085 - DNA, 60 bp, Homo sapiens
  source 1..60: mol_type = other DNA
tgtcttcaacaagatagaaatcaataacaagctggaatttgagtctgcccagttccccaa

SEQ ID NO: 1086 - DNA, 60 bp, Canis sp.
  source 1..60: mol_type = other DNA
tgtcttcaacaagatagaaatcaagaacacagtggaatttgagtcttctcagtaccctaa

SEQ ID NO: 1087 - DNA, 60 bp, Felis catus
  source 1..60: mol_type = other DNA
tgtcttcaacaagacagaaatcaagggcaatgtggaatttgagtcttcccagttccccaa

SEQ ID NO: 1088 - DNA, 60 bp, Equus caballus
  source 1..60: mol_type = other DNA
tgtcttcaacaagatggaaatcaagggcaacgtggaatttgagtctgcaatgtaccccaa

SEQ ID NO: 1089 - DNA, 60 bp, Mus musculus
  source 1..60: mol_type = other DNA
ctggtacatcagcacctcacaagcagagcacaagcctgtcttcctgggaaacaacagtgg

SEQ ID NO: 1090 - DNA, 60 bp, Homo sapiens
  source 1..60: mol_type = other DNA
ctggtacatcagcacctctcaagcagaaaacatgcccgtcttcctgggagggaccaaagg

SEQ ID NO: 1091 - DNA, 60 bp, Canis sp.
  source 1..60: mol_type = other DNA
ctggtacatcagcacctctcaagtcgaaggaatgcctgtcttcctaggaaataccagagg

SEQ ID NO: 1092 - DNA, 60 bp, Felis catus
  source 1..60: mol_type = other DNA
ctggtacatcagcacctctcaagcagaagaaatgcctgtcttcctaggaaataccaaagg

SEQ ID NO: 1093 - DNA, 60 bp, Equus caballus
  source 1..60: mol_type = other DNA
ctggtacatcagcacctctcaagcagaaaaaaagcctgtcttcctaggaaataccagagg

SEQ ID NO: 1094 - DNA, 29 bp, Homo sapiens
  source 1..29: mol_type = other DNA
gttcctccattgatcatctgtctctgaat

SEQ ID NO: 1095 - DNA, 29 bp, Homo sapiens
  source 1..29: mol_type = other DNA
aatccttctatcatgtaagctatggccca

SEQ ID NO: 1096 - DNA, 29 bp, Canis sp.
  source 1..29: mol_type = other DNA
aattgaccatctctctctgaatcaggtaa

SEQ ID NO: 1097 - DNA, 29 bp, Canis sp.
  source 1..29: mol_type = other DNA
aaagacatcccagcttaccttcaaggaaa

SEQ ID NO: 1098 - DNA, 29 bp, Canis sp.
  source 1..29: mol_type = other DNA
caaagtatagttcgacaaacaggaggaaa

SEQ ID NO: 1099 - DNA, 29 bp, Canis sp.
  source 1..29: mol_type = other DNA
ttacctcatgactgctgcattacagaatt

SEQ ID NO: 1100 - DNA, 29 bp, Mus musculus
  source 1..29: mol_type = other DNA
acagtatcagcaacgtcaagcaacgggaa

SEQ ID NO: 1101 - DNA, 29 bp, Mus musculus
  source 1..29: mol_type = other DNA
cagtatcagcaacgtcaagcaacgggaag

SEQ ID NO: 1102 - DNA, 29 bp, Mus musculus
  source 1..29: mol_type = other DNA
agaccttcactgaagatgacctgcagtcc

SEQ ID NO: 1103 - DNA, 29 bp, Mus musculus
  source 1..29: mol_type = other DNA
aatccttctatgatgcaagctatggctca

SEQ ID NO: 1104 - DNA, 29 bp, Homo sapiens
  source 1..29: mol_type = other DNA
agctgatggccctaaacagatgaaggtaa

SEQ ID NO: 1105 - DNA, 29 bp, Homo sapiens
  source 1..29: mol_type = other DNA
catccagctacgaatctccgaccaccact

SEQ ID NO: 1106 - DNA, 29 bp, Homo sapiens
  source 1..29: mol_type = other DNA
agaacctatcttcttcgacacatgggata

SEQ ID NO: 1107 - DNA, 29 bp, Homo sapiens
  source 1..29: mol_type = other DNA
cttcttcgacacatgggataacgaggctt

SEQ ID NO: 1108 - DNA, 29 bp, Homo sapiens
  source 1..29: mol_type = other DNA
gcacctgtacgatcactgaactgcacgct

SEQ ID NO: 1109 - DNA, 29 bp, Homo sapiens
  source 1..29: mol_type = other DNA
aggccgcgtcagttgttgtggccatggac

SEQ ID NO: 1110 - DNA, 29 bp, Homo sapiens
  source 1..29: mol_type = other DNA
tctccgcagtgctccttccaggacctgga

SEQ ID NO: 1111 - DNA, 29 bp, Canis sp.
  source 1..29: mol_type = other DNA
agctgatggccctggaaatgtgaaggtga

SEQ ID NO: 1112 - DNA, 29 bp, Canis sp.
  source 1..29: mol_type = other DNA
tctcccaccagctctgtaacaagagtctg

SEQ ID NO: 1113 - DNA, 29 bp, Mus musculus
  source 1..29: mol_type = other DNA
gcacatcaacaagagcttcaggcaggcag

SEQ ID NO: 1114 - DNA, 29 bp, Mus musculus
  source 1..29: mol_type = other DNA
ggaccttccaggatgaggacatgagcacc

SEQ ID NO: 1115 - DNA, 29 bp, Mus musculus
  source 1..29: mol_type = other DNA
tgacctgggctgtcctgatgagagcatcc

SEQ ID NO: 1116 - protein, 6 aa, Canis sp.
DHLSLN

SEQ ID NO: 1117 - protein, 6 aa, Canis sp.
MSCDPL

SEQ ID NO: 1118 - protein, 6 aa, Canis sp.
TSQLTF

SEQ ID NO: 1119 - protein, 6 aa, Canis sp.
IMKPRS

SEQ ID NO: 1120 - protein, 6 aa, Canis sp.
LNDNLN

SEQ ID NO: 1121 - protein, 6 aa, Canis sp.
QSIVRQ

SEQ ID NO: 1122 - protein, 6 aa, Canis sp.
SIVRQT

SEQ ID NO: 1123 - protein, 6 aa, Canis sp.
GGNYLM

SEQ ID NO: 1124 - DNA, 31 bp, Homo sapiens
  source 1..31: mol_type = other DNA
attcagagacagatgatcaaatggaggaact

SEQ ID NO: 1125 - DNA, 31 bp, Homo sapiens
  source 1..31: mol_type = other DNA
tgggccatagcttacatgattagaaggattt

SEQ ID NO: 1126 - DNA, 31 bp, Homo sapiens
  source 1..31: mol_type = other DNA
agctgatggccctaaacagaatgaaggtaag

SEQ ID NO: 1127 - DNA, 33 bp, Homo sapiens
  source 1..33: mol_type = other DNA
agtggtggtcggagattcgtagctggatgccgc

SEQ ID NO: 1128 - DNA, 31 bp, Homo sapiens
  source 1..31: mol_type = other DNA
agaacctatcttcttcgacatgggataacga

SEQ ID NO: 1129 - DNA, 31 bp, Homo sapiens
  source 1..31: mol_type = other DNA
cttcttcgacacatgggataaagaaggctta

SEQ ID NO: 1130 - DNA, 32 bp, Homo sapiens
  source 1..32: mol_type = other DNA
agcgtgcagttcagtgatctacaggtgcatcg

SEQ ID NO: 1131 - DNA, 32 bp, Homo sapiens
  source 1..32: mol_type = other DNA
gtccatggccacaacaactacgcggcctgcct

SEQ ID NO: 1132 - DNA, 31 bp, Homo sapiens
  source 1..31: mol_type = other DNA
tccaggtcctggaaggagcaactgcggagag

SEQ ID NO: 1133 - DNA, 31 bp, Canis sp.
  source 1..31: mol_type = other DNA
aattgaccatctctctctgaaatcaggtaag

SEQ ID NO: 1134 - DNA, 31 bp, Canis sp.
  source 1..31: mol_type = other DNA
aaagacatcccagcttacctttcaaggaaaa

SEQ ID NO: 1135 - DNA, 31 bp, Canis sp.
  source 1..31: mol_type = other DNA
caaagtatagttcgacaaaccaggaggaaat

SEQ ID NO: 1136 - DNA, 31 bp, Canis sp.
  source 1..31: mol_type = other DNA
aattctgtaatgcagcagtccatcaggtaat

SEQ ID NO: 1137 - DNA, 31 bp, Canis sp.
  source 1..31: mol_type = other DNA
agctgatggccctggaaatgaaggtgagacc

SEQ ID NO: 1138 - DNA, 31 bp, Canis sp.
  source 1..31: mol_type = other DNA
cagatccttgttacagagcttggtgggagac

SEQ ID NO: 1139 - protein, 7 aa, Canis sp.
MSDAAMQ

SEQ ID NO: 1140 - protein, 7 aa, Canis sp.
ELRALHL

SEQ ID NO: 1141 - protein, 7 aa, Canis sp.
SDAAMQS

SEQ ID NO: 1142 - protein, 7 aa, Canis sp.
LRALHLN

SEQ ID NO: 1143 - protein, 7 aa, Canis sp.
QLQVSHQ

SEQ ID NO: 1144 - protein, 7 aa, Canis sp.
LQVSHQL

SEQ ID NO: 1145 - protein, 7 aa, Canis sp.
ADGPGNV

SEQ ID NO: 1146 - DNA, 20 bp, Canis sp.
  source 1..20: mol_type = other DNA
actcttgttacagagctggt

SEQ ID NO: 1147 - DNA, 23 bp, Canis sp.
  source 1..23: mol_type = other DNA
actcttgttacagagctggtngg

SEQ ID NO: 1148 - DNA, 23 bp, Canis sp.
  source 1..23: mol_type = other DNA
actcttgttacagagctggtnga

SEQ ID NO: 1149 - DNA, 23 bp, Canis sp.
  source 1..23: mol_type = other DNA
actcttgttacagagctggtnag

(19) United States
(12) Patent Application Publication
(10) Pub. No.: US 2023/0235321 A1
(43) Pub. Date: Jul. 27, 2023

(54) GENE EDITING TO IMPROVE JOINT FUNCTION

(71) Applicant: ORTHOBIO THERAPEUTICS, INC., Edwards, CO (US)

(72) Inventors: Peter J. Millett, Edwards, CO (US); Iain Alasdair Russell, Cambridge (GB); Matthew J. Allen, Swaffham Bulbeck (GB); George Gentsch, Cambridge (GB)

(21) Appl. No.: 17/867,486

(22) Filed: Jul. 18, 2022

Related U.S. Application Data
(60) Provisional application No. 63/222,972, filed on Jul. 17, 2021.
(60) Provisional application No. 63/300,822, filed on Jan. 19, 2022.
(60) Provisional application No. 63/326,571, filed on Apr. 1, 2022.

(51) Int. Cl. C12N 15/11; A61P 19/02; C12N 9/22; A61K 38/46; A61K 31/7105

(52) U.S. Cl. CPC C12N 15/11; A61P 19/02; C12N 9/22; A61K 38/465; A61K 31/7105; C12N 2310/20; C12N 2800/80; C12N 2310/316

(57) ABSTRACT

The present disclosure provides compositions and methods for treating joint disorders that are characterized by an inflammatory component. In some aspects, the compositions and methods are to prevent the progression of osteoarthritis and other arthritides and to treat osteoarthritis and other arthritides in a mammalian joint.

### CROSS REFERENCE TO RELATED APPLICATIONS

This application claims priority to U.S. Provisional Patent Application No. 63/222,972, filed Jul. 17, 2021, U.S. Provisional Patent Application No. 63/300,822, filed Jan. 19, 2022, and U.S. Provisional Patent Application No. 63/326,571, filed Apr. 1, 2022, the contents of which are hereby incorporated by reference, in their entireties, for all purposes.

### FIELD

Compositions and Methods for treating synovial joint dysfunction are described herein. In addition, methods for gene-editing synovial cells and/or synoviocytes, chondrocytes, synovial macrophages, and synovial fibroblasts, and uses of gene-edited synovial cells and/or synoviocytes, chondrocytes, synovial macrophages, and synovial fibroblasts, in the treatment of diseases such as osteoarthritis are disclosed herein.

### REFERENCE TO SEQUENCE LISTING SUBMITTED ELECTRONICALLY

This application contains a Sequence Listing in XML format submitted electronically herewith via EFS-Web. The contents of the XML copy, created on 123994-5001-WO60, is named “123994-5001-WO60” and is 1,034,491 bytes in size. The Sequence Listing is incorporated herein by reference in its entirety.

### BACKGROUND

Treatment of osteoarthritis, degenerative joint disease, and other joint dysfunction is complex and there are few long term options for either symptomatic relief or restoring joint function. Osteoarthritis (OA) is the leading cause of disability due to pain. Neogi, Osteoarthritis Cartilage 2013; 21:1145-53. All mammal species are affected: working animals, domestic pets, and their owners all suffer OA-related discomfort, pain, and disability, depending on the degree of disease progression.

OA is a complex disease characterized by a progressive course of disability. Systemic inflammation is associated with OA and with OA disease progression. Inflammation is driven by increased levels of pro-inflammatory cytokines. New methods and compositions to treat this disease are acutely needed. Disclosed herein are compositions and methods useful for treating OA as well as other inflammatory joint disorders.

### SUMMARY

The present disclosure provides compositions and methods for treating joint disorders that are characterized by an inflammatory component. In some aspects, the compositions and methods are to prevent the progression of osteoarthritis and other arthritides and to treat osteoarthritis and other arthritides in a mammalian joint. According to exemplary embodiments, at least a portion of the joint synovial cells and/or synoviocytes, chondrocytes, synovial macrophages, or synovial fibroblasts are gene-edited to reduce the expression of inflammatory cytokines. In some aspects, at least a portion of the joint synovial cells and/or synoviocytes, chondrocytes, synovial macrophages, or synovial fibroblasts, are gene-edited to reduce the expression of IL-1α, IL-1β, or both IL-1α, IL-1β.

In some embodiments, the gene-editing causes expression of one or more cytokine and/or growth factor genes to be silenced or reduced in at least a portion of the cells comprising a mammalian joint. In some aspects, the cells are synovial cells. In some aspects, the cells are synovial fibroblasts. In some aspects, the cells are synoviocytes. In some aspects, the cells are chondrocytes. In some aspects, the cells are synovial macrophages.

In some embodiments, the one or more cytokine and/or growth factor genes is/are selected from the group comprising IL-1α, and IL-1β.

In some embodiments, the gene-editing comprises the use of a programmable nuclease that mediates the generation of a double-strand or single-strand break at said one or more cytokine and/or growth factor genes.

In some embodiments, the gene-editing comprises one or more methods selected from a CRISPR method, a TALE method, a zinc finger method, and a combination thereof.

In some embodiments, the gene-editing comprises a CRISPR method.

In some embodiments, the CRISPR method is a CRISPR-Cas9 method.

In some embodiments, the gene-editing comprises a TALE method.

In some embodiments, the gene-editing comprises a zinc finger method.

In some embodiments, the gene-editing causes expression of one or more cytokine and/or growth factor genes to be silenced or reduced in at least a portion of the cells comprising the joint. In some embodiments, the portion of cells edited are synoviocytes. In an aspect, the portion of cells edited are synovial fibroblasts. In some embodiments, the portion of cells edited are synoviocytes. In some embodiments, the portion of cells edited are chondrocytes. In some embodiments, the portion of cells edited are synovial macrophages.

In some embodiments, an adeno-associated virus (AAV) delivery system is used to deliver the gene-editing system. In some embodiments, the AAV delivery system is injected into a joint.

Some aspects of the present disclosure provide a pharmaceutical composition for the treatment or prevention of a joint disease or condition comprising a gene-editing system and a pharmaceutically acceptable carrier. In an aspect, the gene-editing system comprises one or more nucleic acids targeting one or more genetic locus selected from the group consisting of IL-1α, IL-1β, TNF-α, IL-6, IL-8, and IL-18.

In some embodiments, the gene-editing system comprises a composition for the treatment or prevention of a joint disease or condition, comprising an RNA-guided nuclease or a nucleic acid encoding an RNA-guided nuclease and at least one guide RNA or a nucleic acid encoding at least one guide RNA targeting an IL-1α, or IL-1β gene, wherein the guide RNA specifically binds a target sequence that is adjacent to a protospacer adjacent motif (PAM) sequence for the Cas9 protein.

In some embodiments, at least one guide RNA targets a human IL-1α and comprises a crRNA sequence having at least 85%, 90%, 95%, or 100% identity to a sequence selected from the group consisting of SEQ ID NOs: 168-187, 298-387, and 681-710.

In some embodiments, at least one guide RNA targets a human IL-1β gene and comprises a crRNA sequence having at least 85%, 90%, 95%, or 100% identity to a sequence selected from the group consisting of SEQ ID NOs: 188-201, 388-496, and 711-740.

In some embodiments, at least one guide RNA targets a canine IL-1α gene and comprises a crRNA sequence having at least 85%, 90%, 95%, or 100% identity to a sequence selected from the group consisting of SEQ ID NOs: 202-216, 552-590, and 741-770.

In some embodiments, at least one guide RNA targets a canine IL-1β gene and comprises a crRNA sequence having at least 85%, 90%, 95%, or 100% identity to a sequence selected from the group consisting of SEQ ID NOs: 217-235, 497-551, and 771-800.

In some embodiments, the gene-editing system comprises one or more lipid nanoparticles (LNP) collectively comprising an RNA-guided nuclease or the nucleic acid encoding the RNA-guided nuclease and at least one guide RNA or the nucleic acid encoding the at least one guide RNA.

In some embodiments, the LNP comprise a first plurality of LNP comprising a first nucleic acid, encapsulating the nucleic acid encoding the RNA-guided nuclease; and a second plurality of LNP comprising a second nucleic acid, encapsulating the nucleic acid encoding the at least one guide RNA.

In some embodiments, the LNP comprise a first plurality of LNP, encapsulating the RNA-guided nuclease and a second plurality of LNP comprising a second nucleic acid, encapsulating the nucleic acid encoding the at least one guide RNA.

In some embodiments, the LNP comprise a single nucleic acid, wherein the single nucleic acid encodes the RNA-guided nuclease and the at least one guide RNA.

In some embodiments, the gene-editing system comprises one or more liposomes collectively comprising the RNA-guided nuclease or the nucleic acid encoding the RNA-guided nuclease and at least one guide RNA or the nucleic acid encoding the at least one guide RNA.

In some embodiments, the nucleic acid encoding the RNA-guided nuclease and/or the nucleic acid encoding the at least one guide RNA are present in a naked state.

In some embodiments, the RNA-guided nuclease in the gene-editing system is a Cas9 protein. In some embodiments, the Cas9 protein is spCas9. In some embodiments, the Cas9 protein is espCas9. In other embodiments, the Cas9 protein is saCas9.

An embodiment provides a method of treating canine lameness, the method comprising administering a gene-editing composition, wherein the composition causes expression of IL-1α and IL-1β to be silenced or reduced in a portion of a lame joint's synoviocytes, chondrocytes, synovial macrophages, or synovial fibroblasts.

An embodiment provides a method for treating a joint disease or condition in a subject in need thereof. In some embodiments, the joint disease or condition is arthritis. In some embodiments, the joint disease or condition is osteoarthritis.

In some embodiments, the gene-editing composition is formulated for parenteral administration. In some embodiments, the gene-editing composition is formulated for intra-articular injection within a joint of a subject.

In some embodiments, the above method further comprises one or more features recited in any of the methods and compositions described herein.

### DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIG. 1A illustrates an agarose gel electrophoresis analysis of 100 ng mouse DNA (gBlocks, Integrated DNA Technologies) designed against the Mus musculus Il1a and Il1b genes, cleaved by 0.5 μg SpyCas9 (TrueCut™ Cas9 protein v2, ThermoFisher Scientific) and 200 ng Phosphorothioate-modified single guide (sg)RNAs targeted against the Il1a gene (#43-46) and Il1b gene (#47-50) in vitro.

FIG. 1B illustrates an agarose gel electrophoresis analysis of 100 ng mouse DNA (gBlocks, Integrated DNA Technologies) designed against the Mus musculus Il1a and Il1b genes, cleaved by 0.5 μg SauCas9 (GeneSnipper™ Cas9, BioVision) and 200 ng Phosphorothioate-modified guide sgRNAs against the Il1a (#51-53) and IL1b (#54-56) genes.

FIGS. 2A, 2B, 2C, and 2D collectively illustrate graphs displaying editing efficiencies of SpyCas9 and SauCas9 used with a range of guide RNA's in J774.2 (“J”) and NIH3T3 (“N”) cells; FIG. 2A: in vivo cleavage of Il1a, edited with 4×sgRNAs (Spy Cas9) in two separate pools (Pool 1 and 2), across two cell lines, NIH 3T3 (“N”), and J774.2 (“J”); FIG. 2B: in vivo cleavage of Il1b, edited with 4×sgRNAs (Spy Cas9) in two separate pools (Pool 1 and 2), across two cell lines, NIH 3T3 (“N”), and J774.2 (“J”); FIG. 2C: in vivo cleavage of Il1a, edited with 3× sgRNAs (Sau Cas9) in two separate pools (Pool 1 and 2), across two cell lines, NIH 3T3 (“N”), and J774.2 (“J”); FIG. 2D: in vivo cleavage of Il1b, edited with 3× sgRNAs (saCas9) in two separate pools (Pool 1 and 2), across two cell lines, NIH 3T3 (“N”), and J774.2 (“J”); editing efficiencies determined using deconvolution of Sanger sequencing traces (ICE tool, Synthego) of each pool.

FIG. 3 illustrates GFP expression measured using the IVIS system. Flux values were based on a region of interest centred on the animal's injected knee joint. Data are presented as mean (SD) for four specimens per group.

FIG. 4 illustrates the design of a study as described in Example 5 of the present disclosure.

FIG. 5 illustrates the in-life outcome measurements obtained in a study as described in Example 5 of the present disclosure.

FIG. 6 illustrates the change in body weight of mice treated with an intra-articular (IA) injection of PBS, AAV-6 with a scrambled vector, AAV-6 with CRISPR-Cas guides 1 and 2, AAV-5 with a scrambled vector, or AAV-5 with CRISPR-Cas guides 1 and 2 in a study as described in Example 5 of the present disclosure.

FIGS. 7A and 7B collectively illustrate (A) change in knee caliper measurements from baseline of mouse joints over time, and (B) mean difference in ankle caliper measurements with AUC in mice treated with an intra-articular (IA) injection of PBS, AAV-6 with a scrambled vector, AAV-6 with CRISPR-Cas guides 1 and 2, AAV-5 with a scrambled vector, or AAV-5 with CRISPR-Cas guides 1 and 2 in a study as described in Example 5 of the present disclosure.

FIGS. 8A and 8B collectively illustrate (A) change in von Frey measurements, and (B) mean absolute threshold in von Frey measurements obtained from mice treated with an intra-articular (IA) injection of PBS, AAV-6 with a scrambled vector, AAV-6 with CRISPR-Cas guides 1 and 2, AAV-5 with a scrambled vector, or AAV-5 with CRISPR-Cas guides 1 and 2 in a study as described in Example 5 of the present disclosure.

FIG. 9 illustrate results of a qPCR assay for IL-1β expression in synovial fluid obtained from mice treated with an intra-articular (IA) injection of PBS, AAV-6 with a scrambled vector, AAV-6 with CRISPR-Cas guides 1 and 2, AAV-5 with a scrambled vector, or AAV-5 with CRISPR-Cas guides 1 and 2 in a study as described in Example 5 of the present disclosure.

FIGS. 10A, 10B, 10C, and 10D collectively illustrate immunohistochemistry for murine IL-1β in synovial tissue of MSU injected animals (A, B) pre-treated with PBS, and (C, D) treated with CRISPR. FIGS. 10B and 10D show isotype controls for each of FIGS. 10A and 10C, respectively.

FIGS. 11A, 11B, and 11C collectively illustrate an alignment between the mouse, human, equine, feline, and canine IL-1 alpha genes.

FIGS. 12A, 12B, 12C, and 12D collectively illustrate an alignment between the mouse, human, equine, feline, and canine IL-1 beta genes.

FIGS. 13A, 13B, 13C, and 13D collectively illustrate example CRISPR/Cas9 crRNA sequences designed for editing the human IL-1 alpha gene.

FIGS. 14A, 14B, 14C, 14D, and 14E collectively illustrate example CRISPR/Cas9 crRNA sequences designed for editing the human IL-1 beta gene.

FIGS. 15A, 15B, and 15C collectively illustrate example CRISPR/Cas9 crRNA sequences designed for editing the canine IL-1 alpha gene.

FIGS. 16A and 16B collectively illustrate example CRISPR/Cas9 crRNA sequences designed for editing the canine IL-1 beta gene.

FIGS. 17A, 17B, 17C, and 17D collectively illustrate the results of cell-based and in-silico gene editing analysis of crRNA sequences targeting the human IL-1 alpha gene (FIG. 7A), human IL-1 beta gene (FIG. 7B), canine IL-1 alpha gene (FIG. 7C), and canine IL-1 beta gene (FIG. 7D), as described in Example 8. ° CRISPR cut position within the translation frame of amino acids (AA). * Optimized score from Doench, Fusi et al. (2016). This score is optimized for 20 bp guides with NGG. Score spans from 0 to 100. Higher is better. ** Specificity score from Hsu et al. (2013). Score spans from 0 to 100. Higher is better. *** This score is based on experiments in U2OS. A high precision score (>0.4) implies that DNA repair outcomes are uniform and enriched for just a handful of unique genotypes. **** This score is based on experiments in U2OS. A high (>80%) frameshift frequency will tend to knock a protein-coding gene out of frame. The typical genomic frameshift frequency is above 66% because 1-bp insertions and 1-2 bp deletions are particularly common repair outcomes. \hat{ } Combined score=(Off-target score+Precision score*100+Frameshift)/3. † Pipe symbol ‘|’ indicates CRISPR cut site. Curly braces ‘{ }’ indicate insertion. Hyphen ‘-’ indicates deletion. $ Potential off-target sites. Scoring according to Hsu et al. (2013). The on-target site has a score of 100.

FIGS. 18A, 18B, 18C, and 18D collectively illustrate canine IL-1 alpha (FIGS. 18A and 18B) and canine IL-1 beta (FIGS. 18C and 18D) release from non-edited (control) and double IL-1α/IL-1β KO (edited) canine chondrocytes 6 hours (FIGS. 18A and 18C) and 24 hours (FIGS. 18B and 18D) after exposure to PBS or LPS, as described in Example 9.

FIGS. 19A, 19B, 19C, and 19D collectively illustrate human IL-1 alpha (FIGS. 19A and 19B) and canine IL-1 beta (FIGS. 19C and 19D) release from non-edited (control) and double IL-1α/IL-1β KO (edited) canine chondrocytes 6 hours (FIGS. 19A and 19C) and 24 hours (FIGS. 19B and 19D) after exposure to PBS or LPS, as described in Example 9.

FIG. 20 illustrates the results of a tissue-specific splicing and expression analysis of human IL1A (IL-1a) gene.

FIG. 21 illustrates the results of a tissue-specific splicing and expression analysis of human IL1B (IL-1b) gene.

FIG. 22 illustrates the results of an in silico analysis of human IL-1a- and IL-1b-targeted gRNA targeting domains. On-target score (see Doench et al.) is optimized for 20-bp gRNA with NGG protospacer adjacent motif (PAM). Score spans from 0 to 1. Precision score is based on experiments in U2OS cells. A high precision score (>0.4) implies that DNA repair outcomes are uniform and enriched for just a handful of unique genotypes. Frameshift percentage is based on experiments in U2OS cells. A high (>80%) frameshift frequency will tend to knock a protein-coding gene out of frame. The typical genomic frameshift frequency is above 66% because 1-bp insertions and 1-2 bp deletions are particularly common repair outcomes. Off-target score from CRISPOR assess the number of matches in the genome with a given number of mismatches. Mismatches in Seed sequence have a more deleterious effect.

Numbers #237:
(SEQ ID NO: 498)
tgatggccctggaaatgtga;
#238:
(SEQ ID NO: 630)
ggtggtcggagattcgtagc;
#248:
(SEQ ID NO: 717)
acctatcttcttcgacacat;
#249
(SEQ ID NO: 60)
cttcgacacatgggataacg;
#250
(SEQ ID NO: 61)
gtgcagttcagtgatcgtac;
#259
(SEQ ID NO: 390)
catggccacaacaactgacg;
#260
(SEQ ID NO: 440)
aggtcctggaaggagcactg.

FIGS. 23A, 23B, 23C, and 23D collectively illustrate results of splicing and functional analyses on canine IL1A (IL-1α) and IL1B (IL-1b) genes. The reference canine genome assembly (CanFam3.1) was used for these analyses. gRNA designs may be tailored to the individual breed, as necessary.

FIG. 24 illustrates the results of an in silico analysis of canine IL-1a- and IL-1b-targeted gRNA targeting domains. Numbers #239: tgaccatctctctctgaatc (SEQ ID NO:552); #240: gacatcccagcttaccttca (SEQ ID NO:554); #251: agtatagttcgacaaacagg (SEQ ID NO:578); #252 tctgtaatgcagcagtcatg (SEQ ID NO:579); #241 tgatggccctggaaatgtga (SEQ ID NO:781); #242 actcttcttgagggtaagct (SEQ ID NO:875).

FIGS. 25A, 25B, and 25C collectively illustrate knockdown efficacy of selected gRNA targeting domains in human chondrocytes (FIG. 25A), canine chondrocytes (FIG. 25B) and synoviocytes (FIG. 25C). Genomic DNA was collected from pooled cells at 8-10 days post-administration prior to sequencing analysis.

FIG. 26 illustrates the results of an in silico analysis of the off-target effects for lead candidate gRNA targeting domains in canine cells. sgRNA #240-1: cacatccctccttaccttca (SEQ ID NO:934); sgRNA #240-2: taaataccagcccaccttca (SEQ ID NO:935); sgRNA #240-3: gtggacccagcttaccttca (SEQ ID NO:936); sgRNA #242-1: acttttgtttcagagctggt (SEQ ID NO:937); sgRNA #242-2: cctcatgctacagagctggt (SEQ ID NO:922); sgRNA #242-3: gtgcttgttacagagctggt (SEQ ID NO:923).

FIGS. 27A and 27B collectively illustrate the efficacy of enhanced-specificity Cas9 (espCas9) to abrogate the off-target editing of sgRNA 242 in canine cells. FIG. 27A shows the effects with canonical spCas9, resulting in strong on-target editing but high off-target activity also. FIG. 27B shows that espCas9 maintain the high on-target efficiency without off-target effects.

FIGS. 28A and 28B collectively illustrate editing activity for sgRNA targeting targeting IL-1α and IL-1b in human (A) and canine (B) chondrocytes. For FIG. 28A, numbers #235:

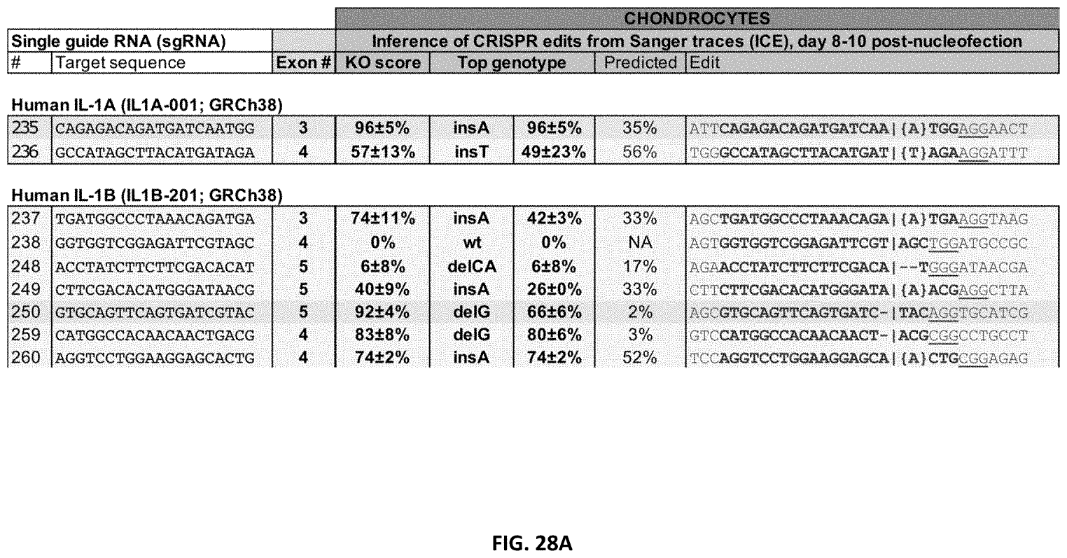

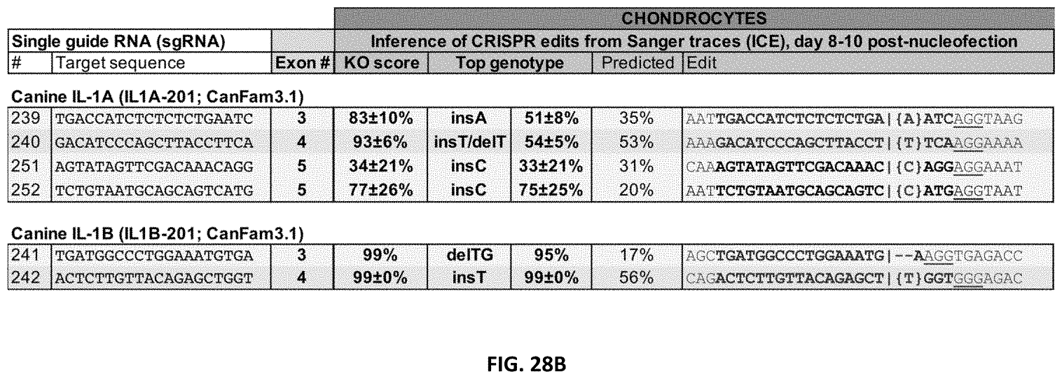

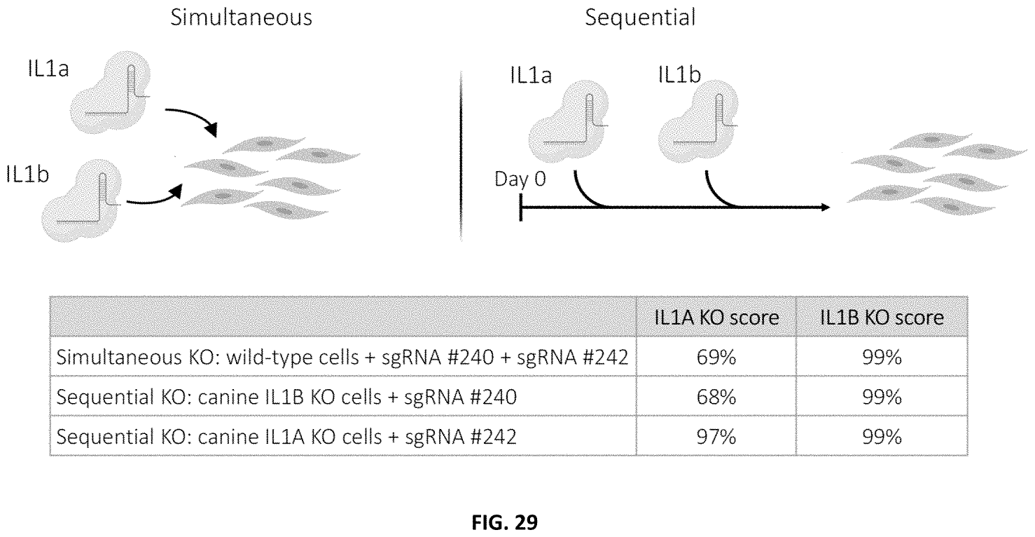

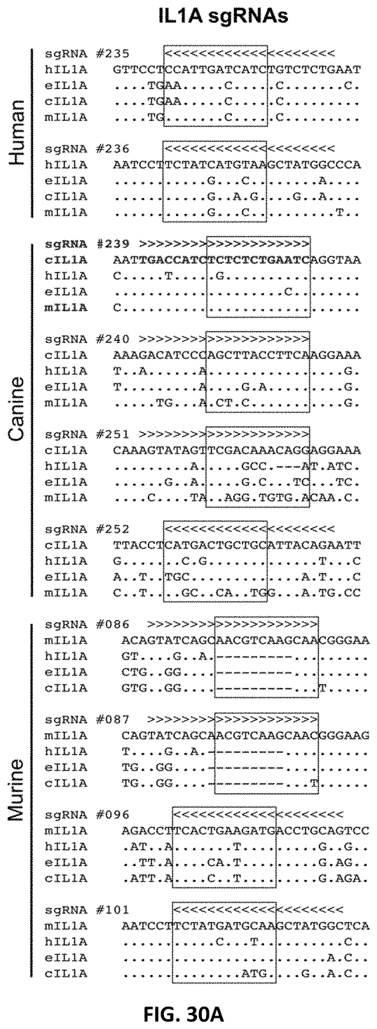

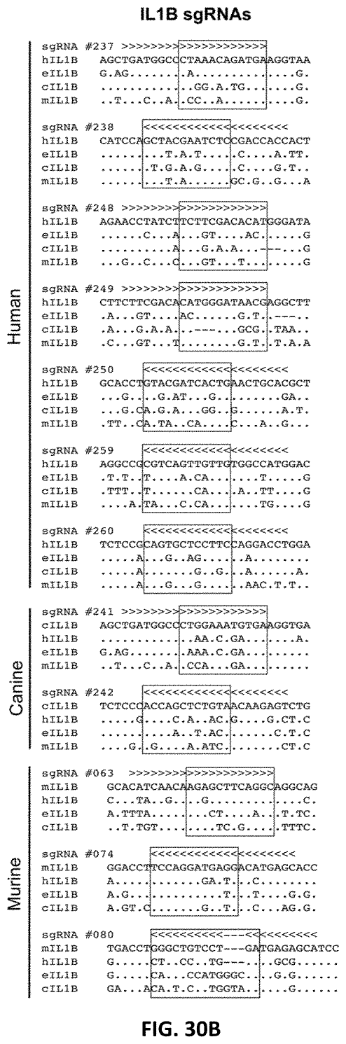

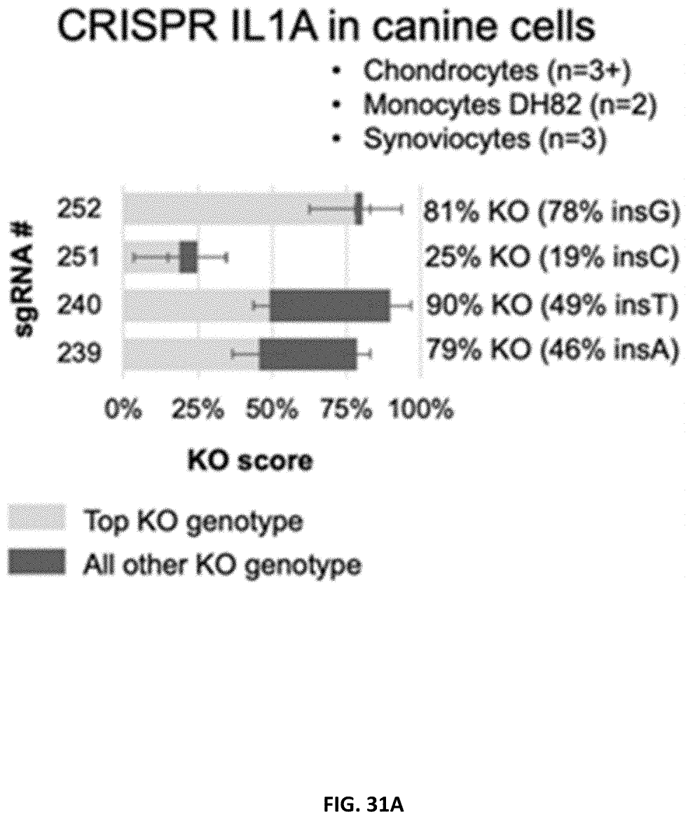

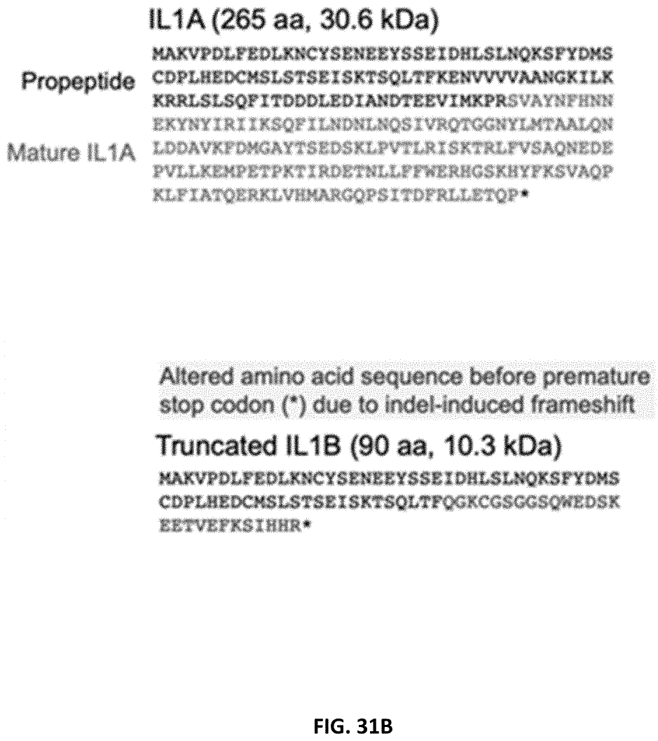

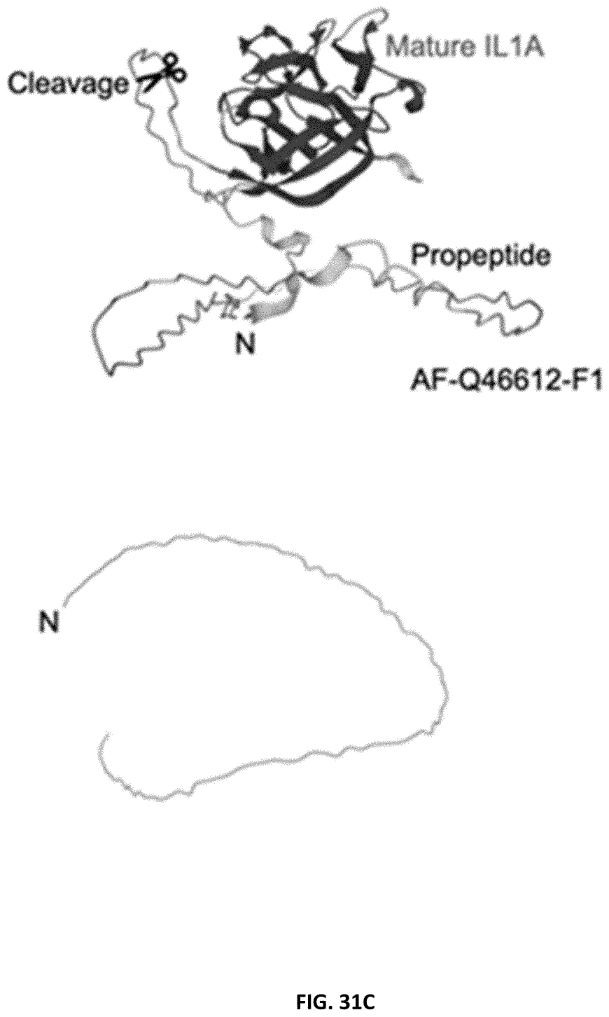

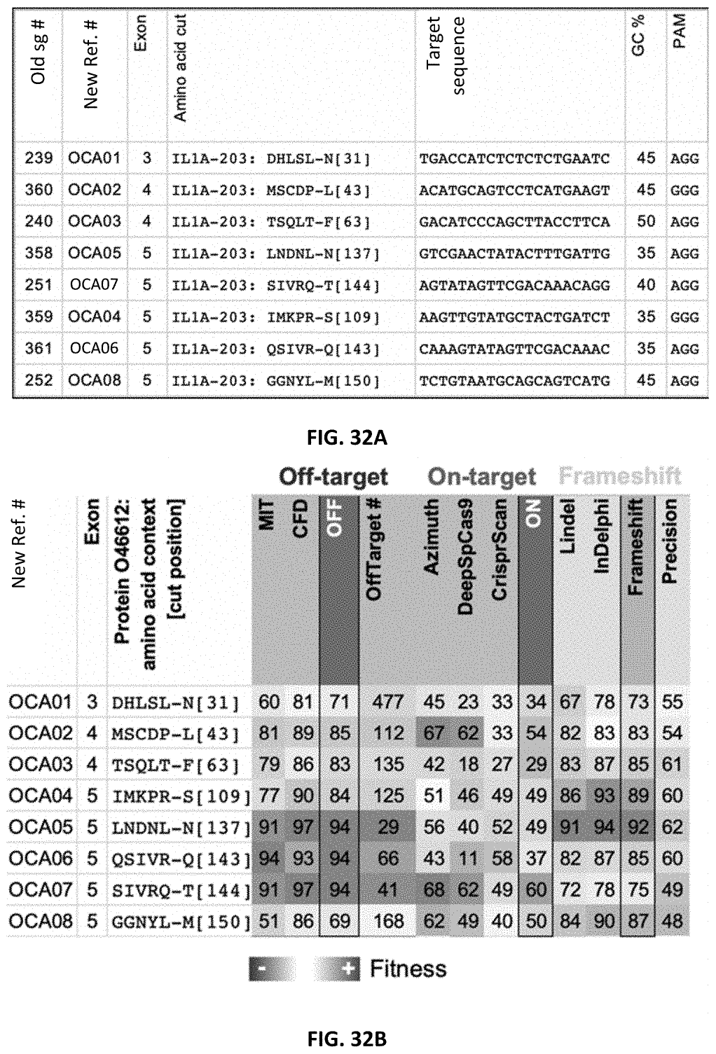

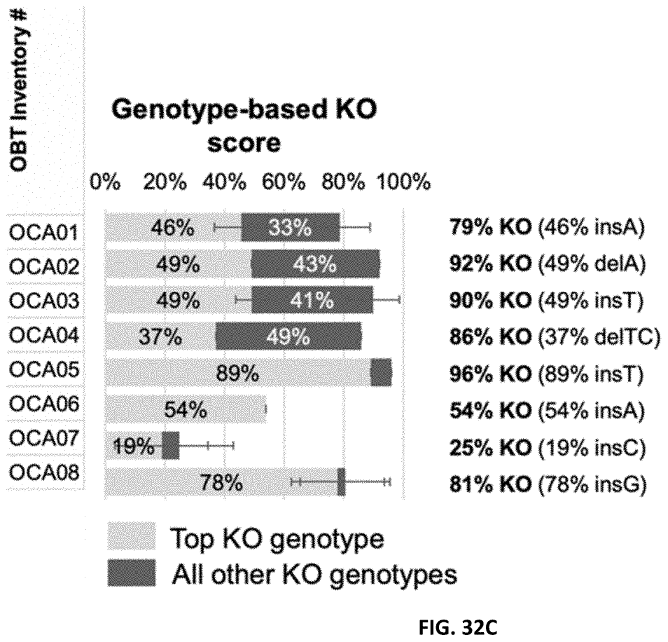

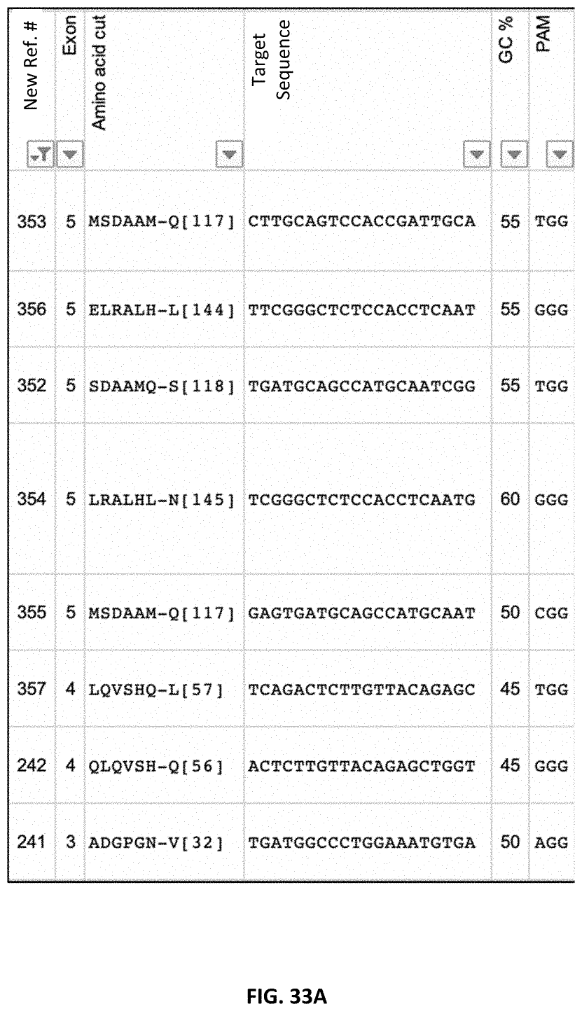

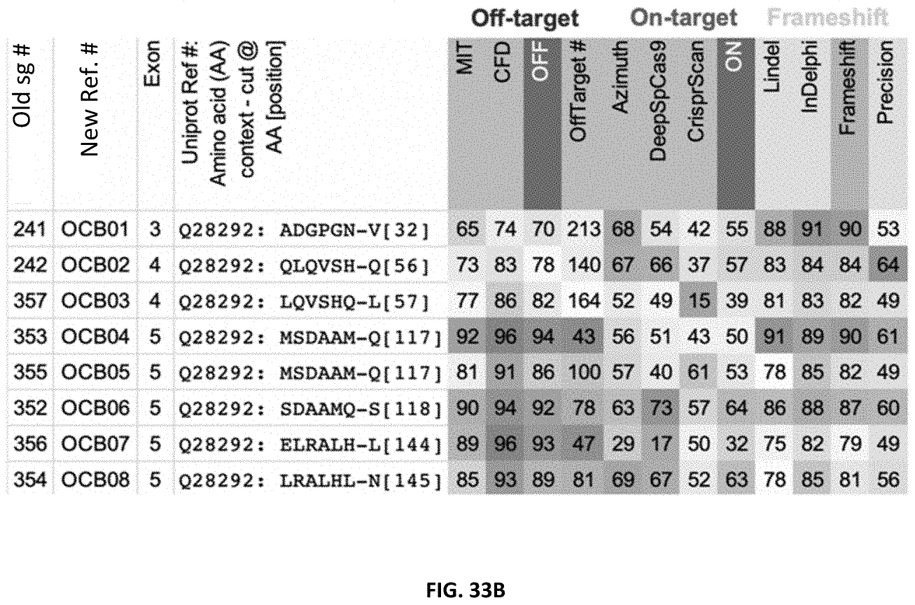

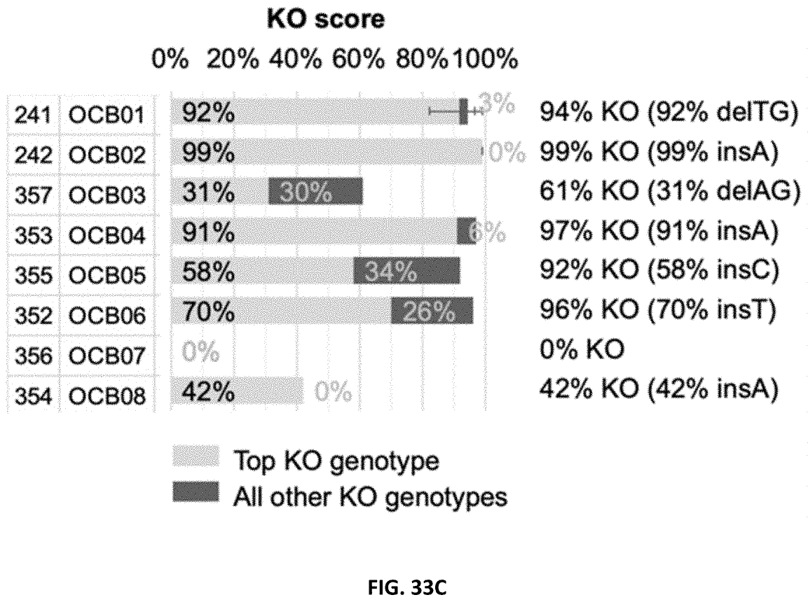

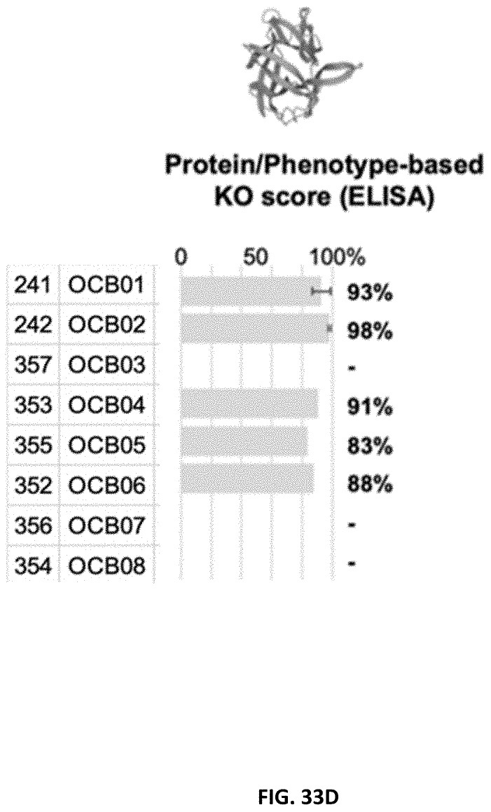

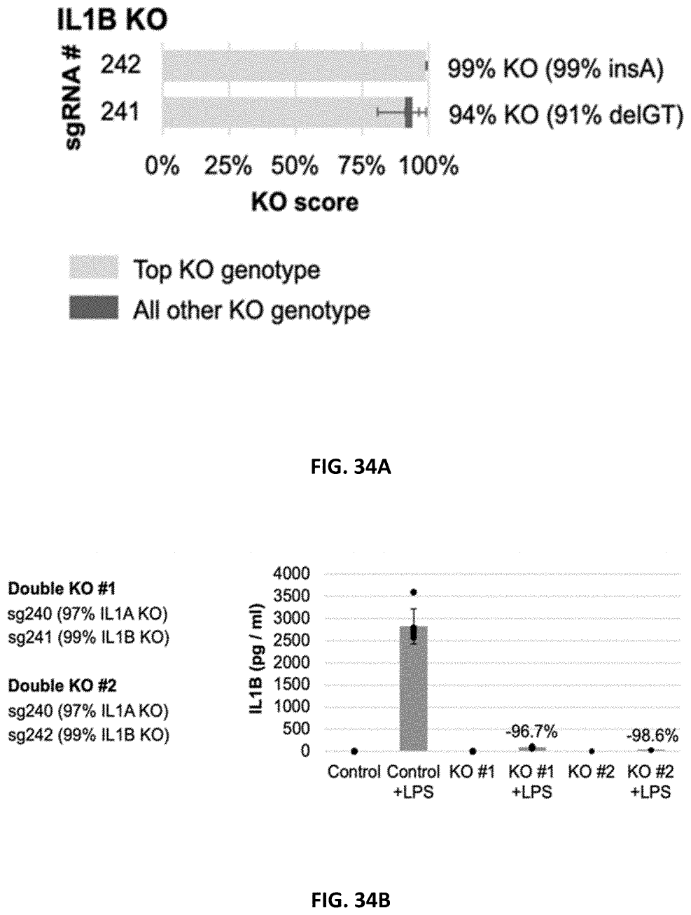

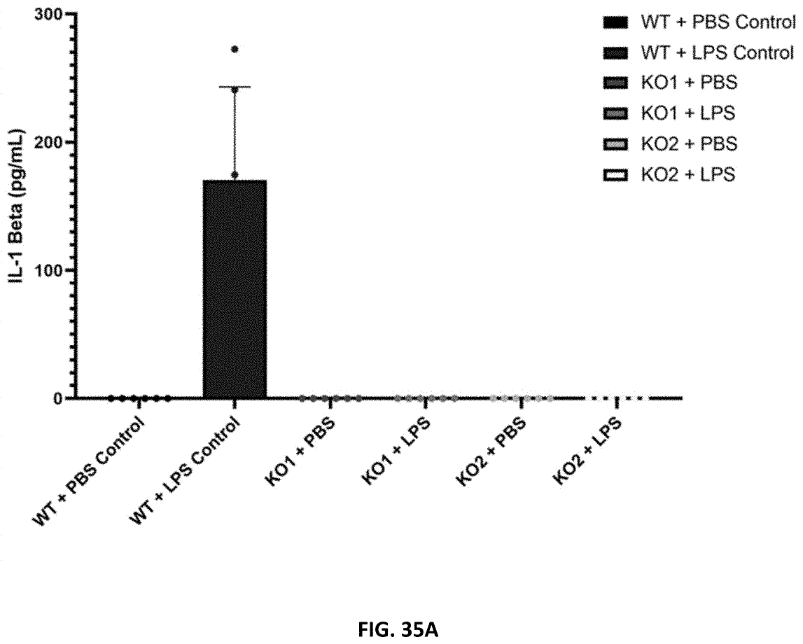

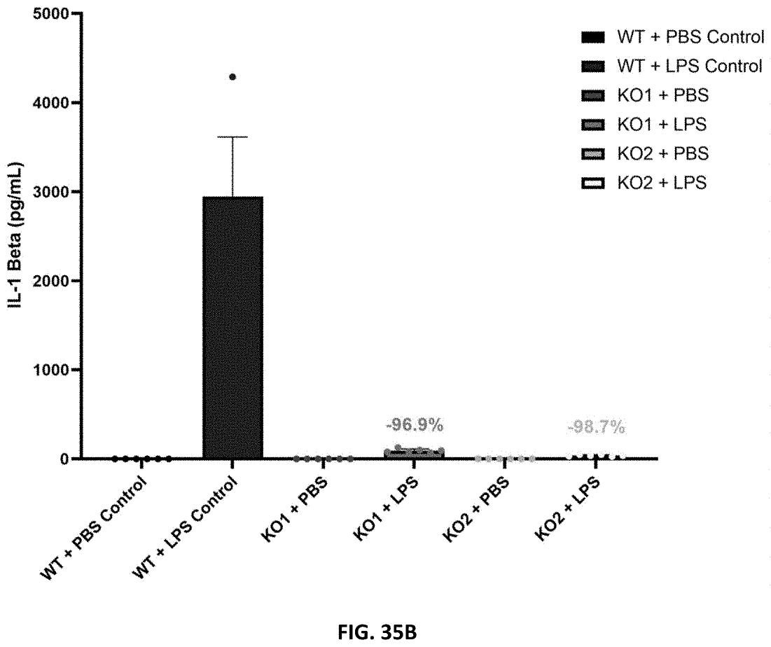

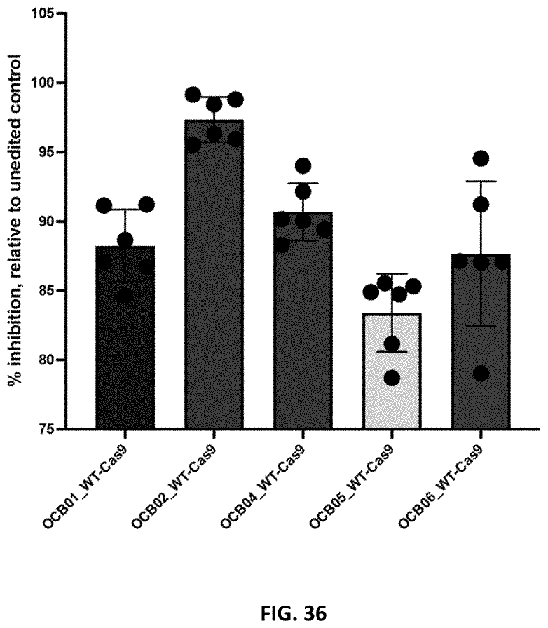

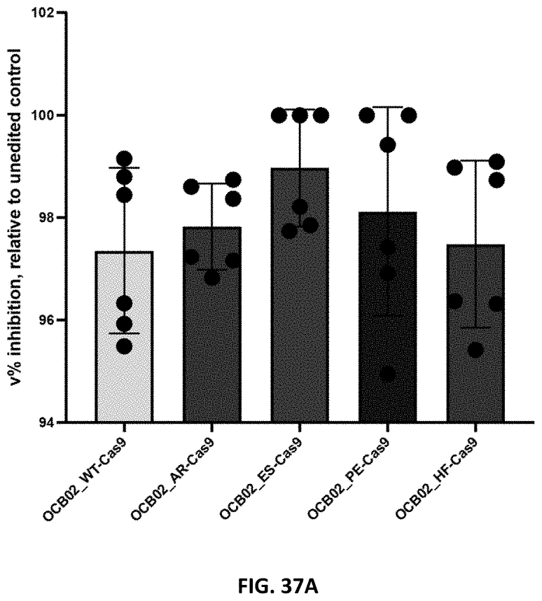

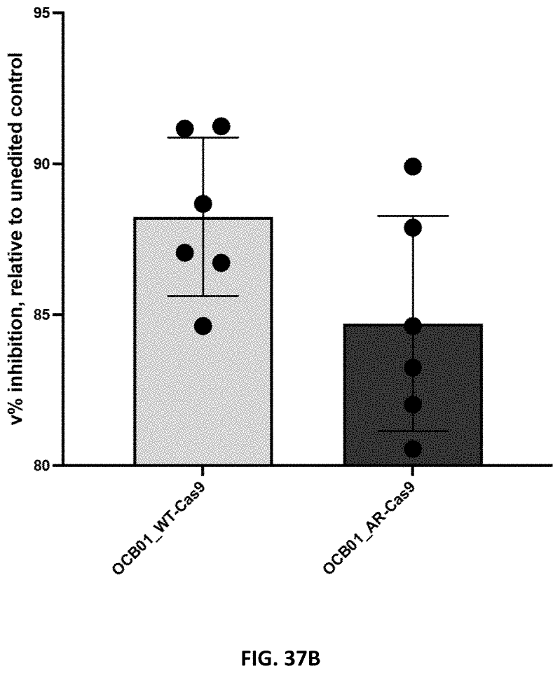

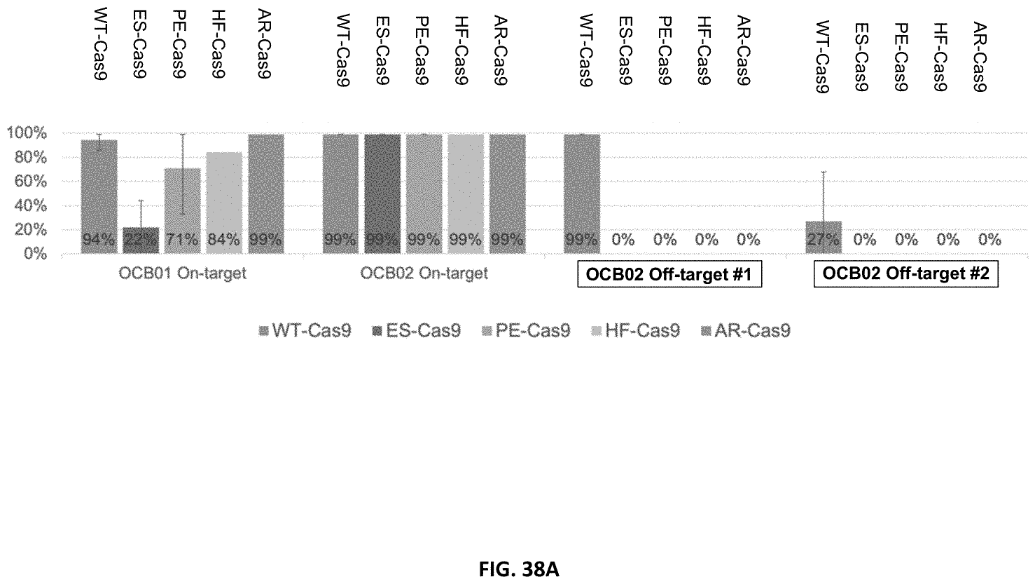

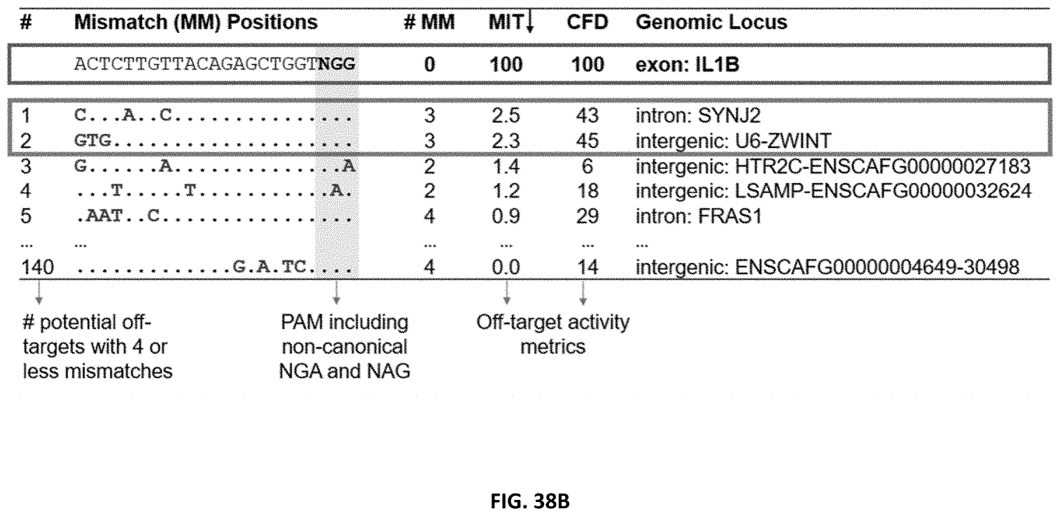

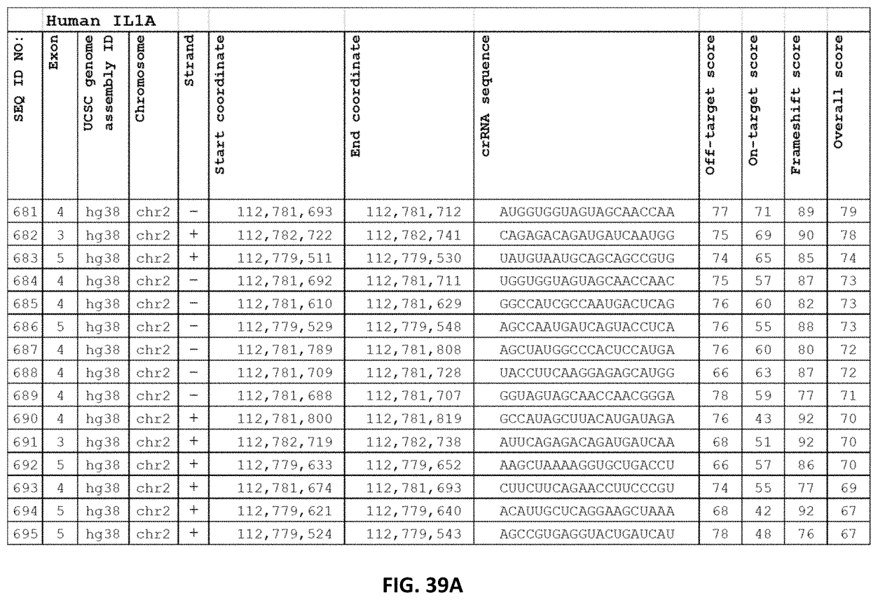

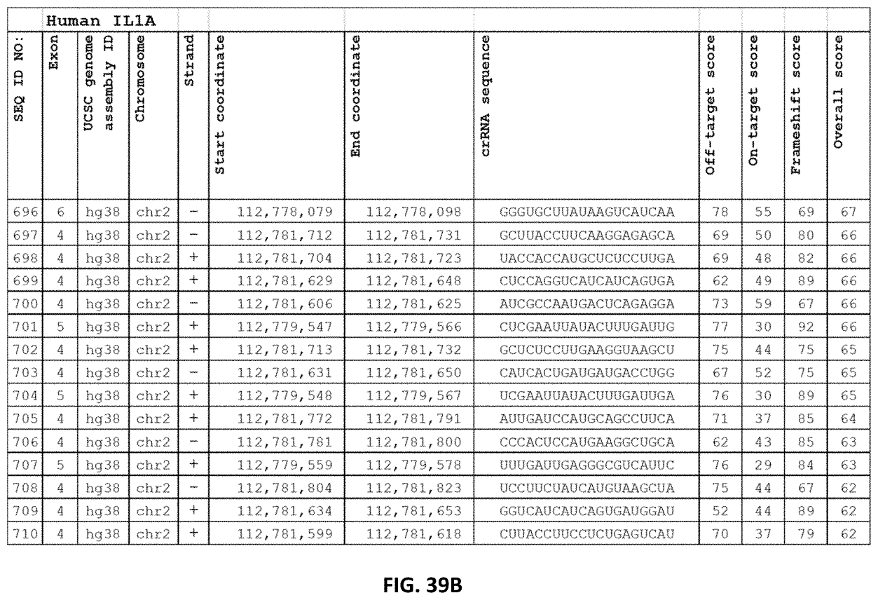

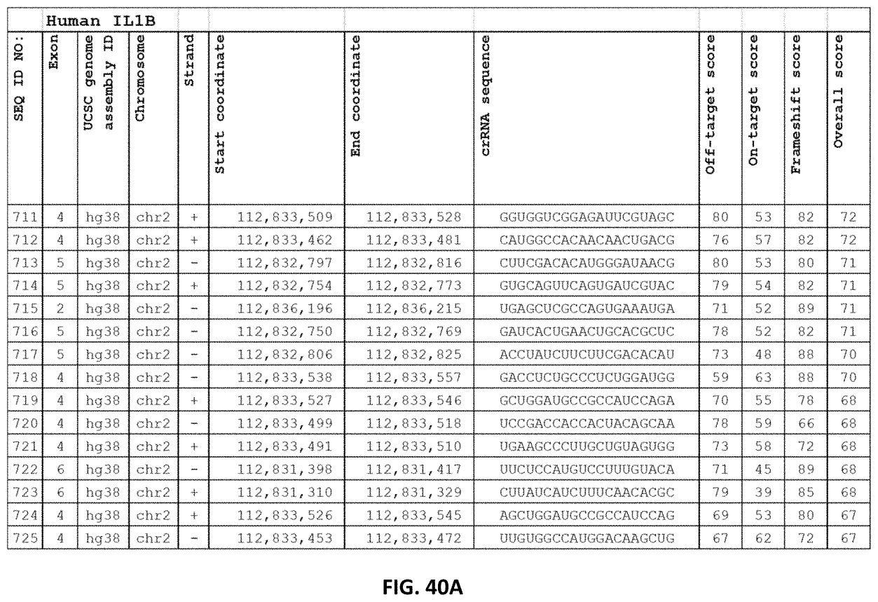

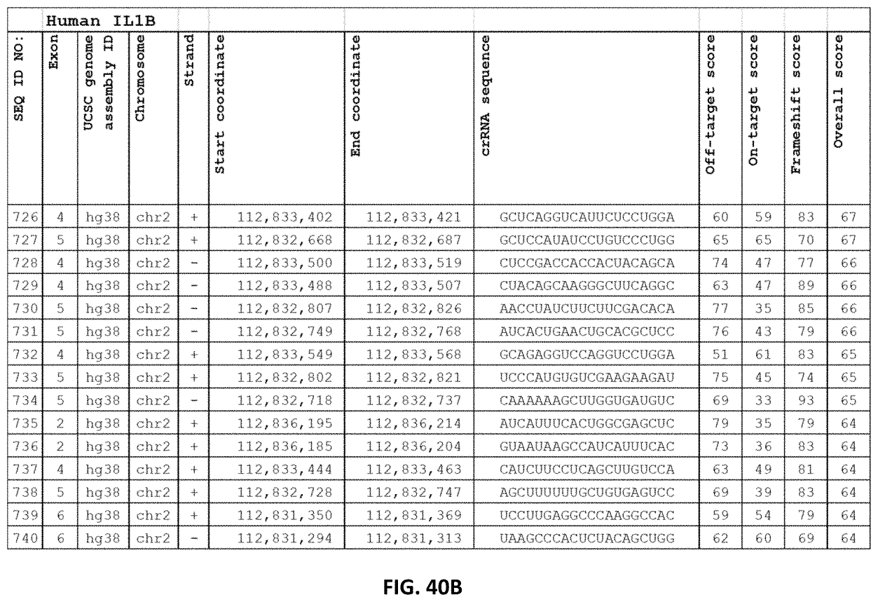

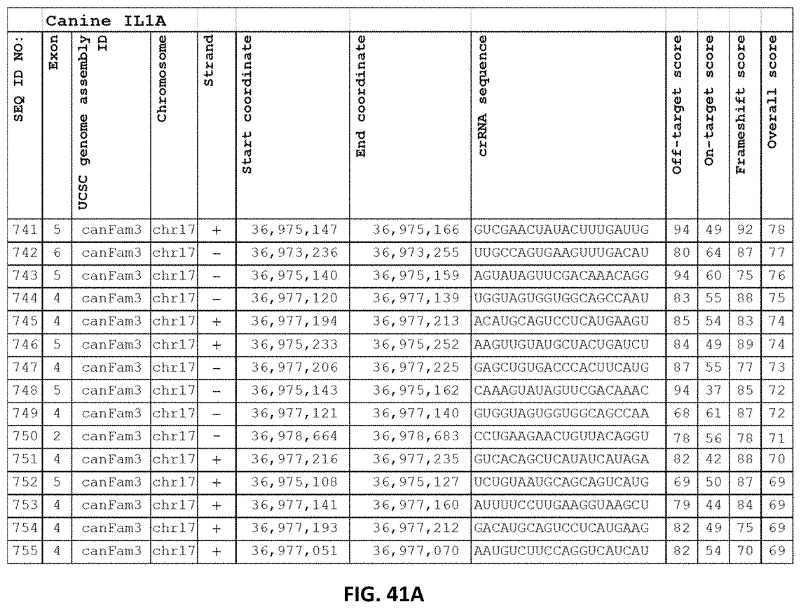

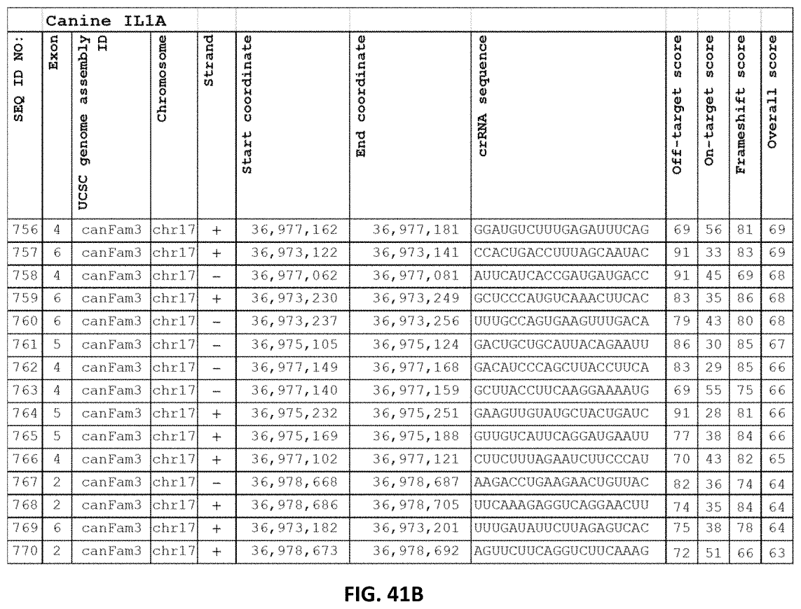

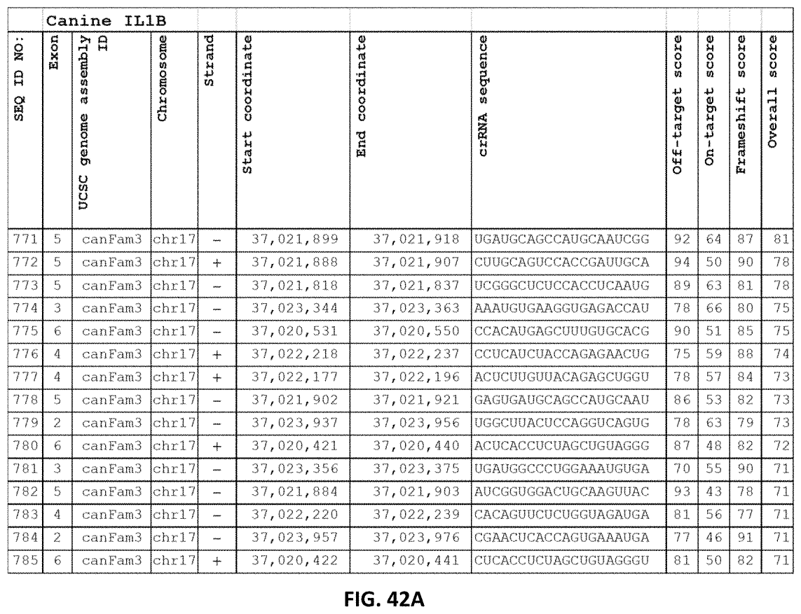

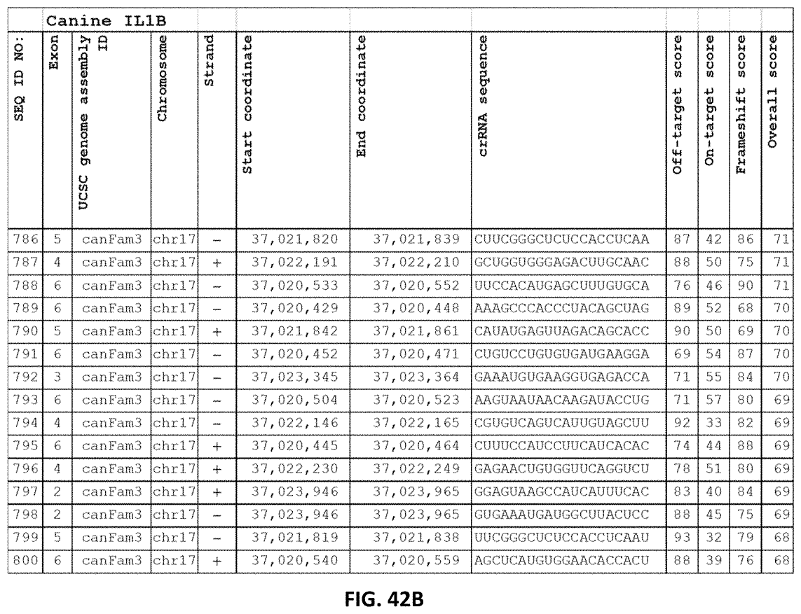

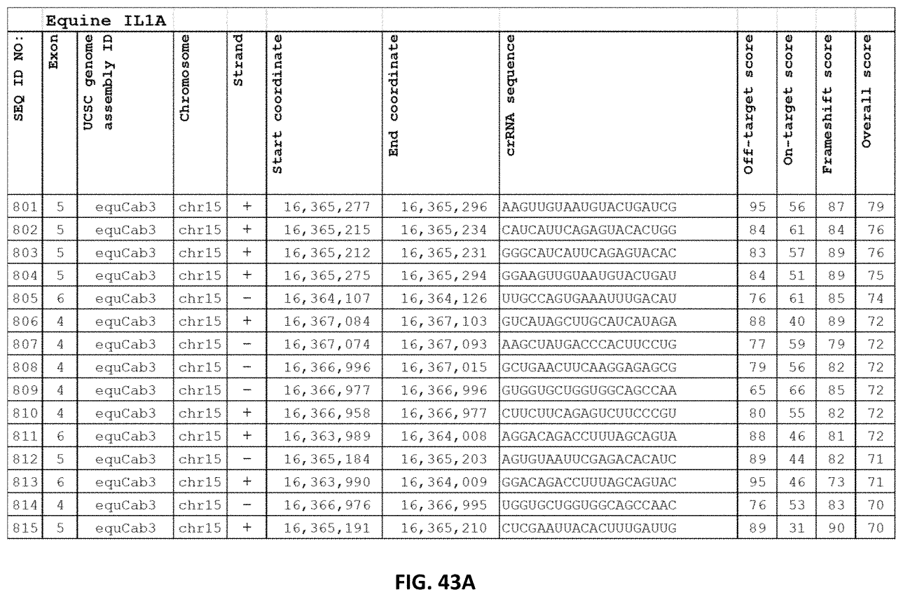

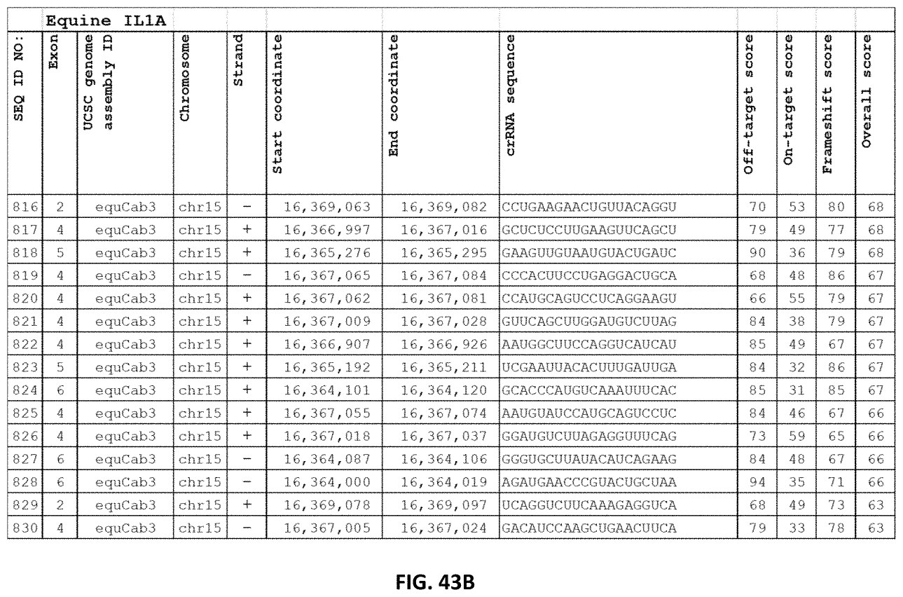

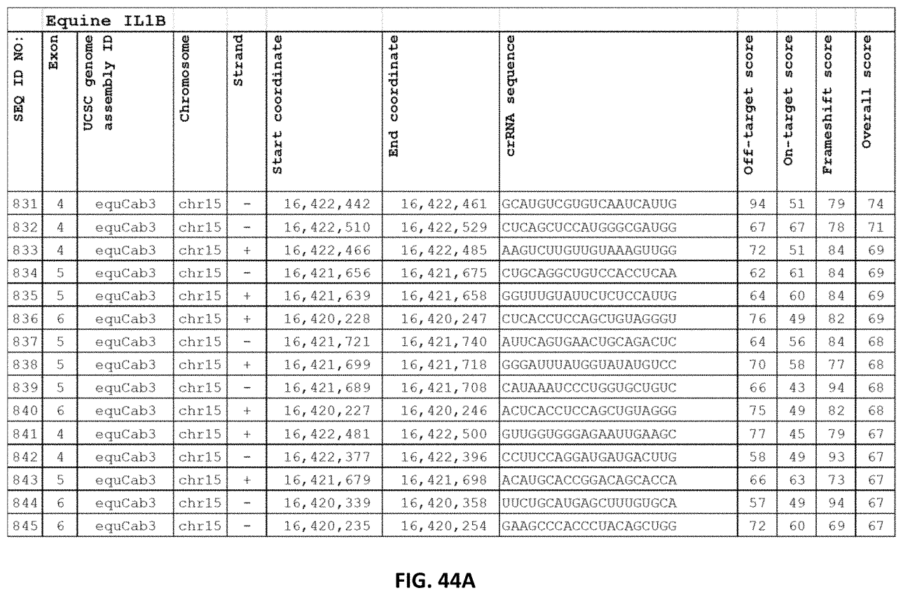

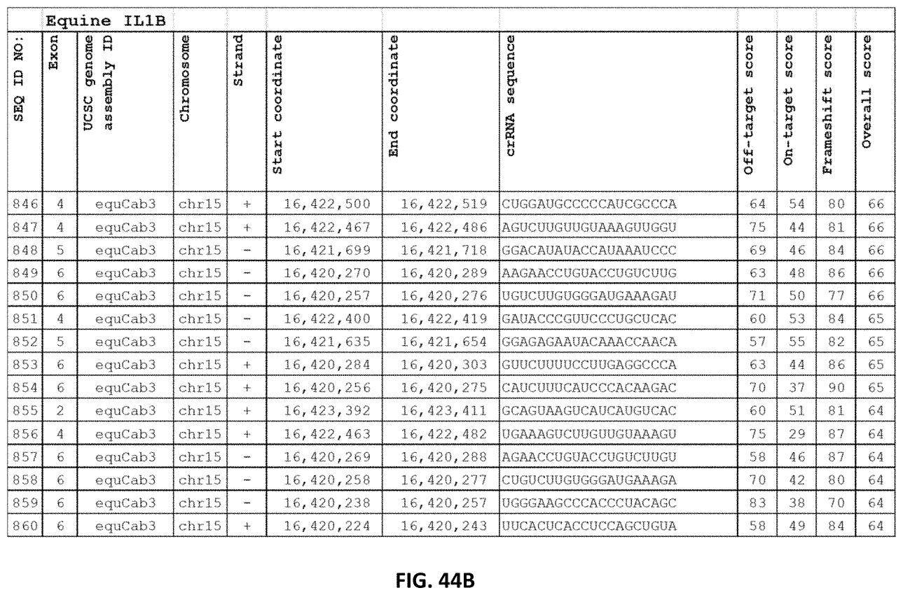

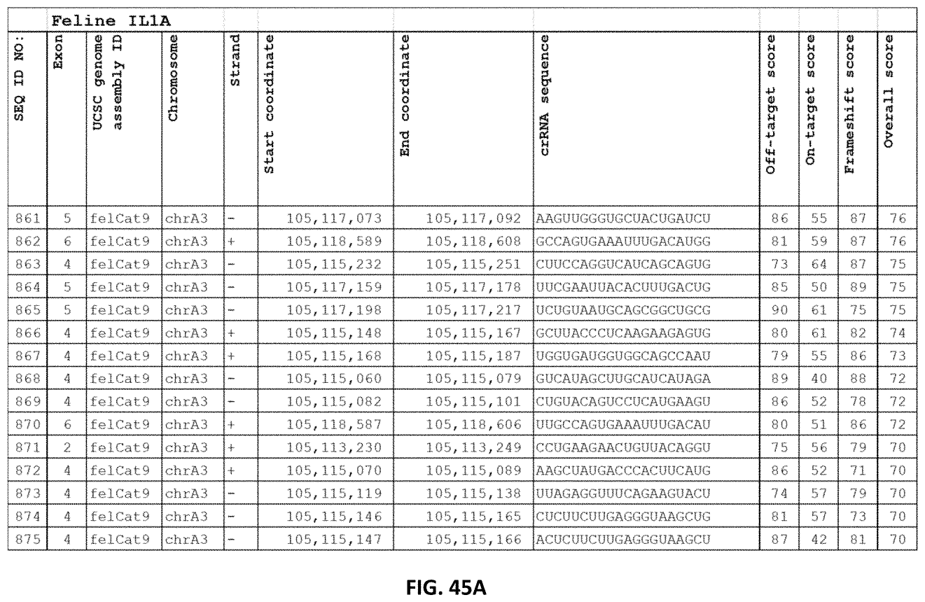

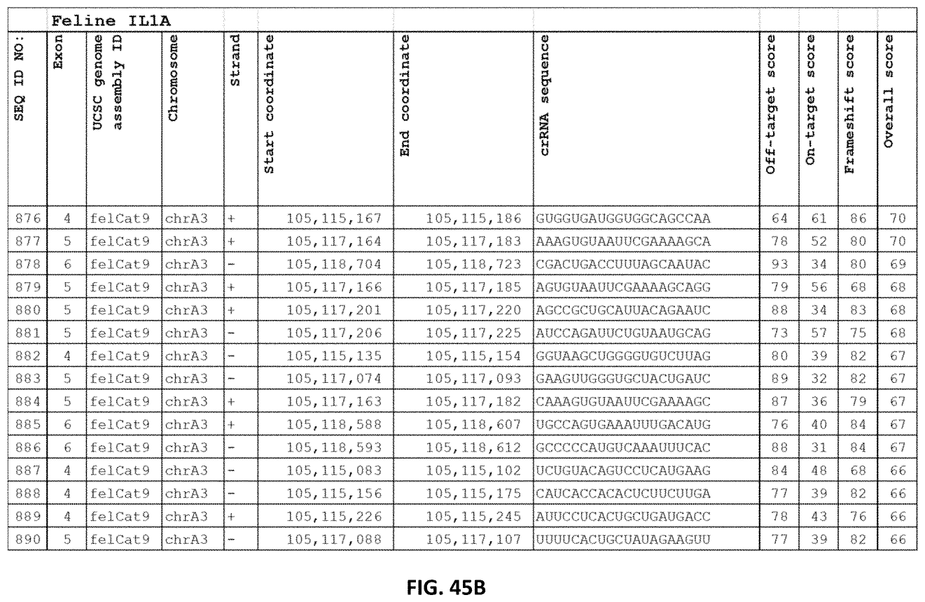

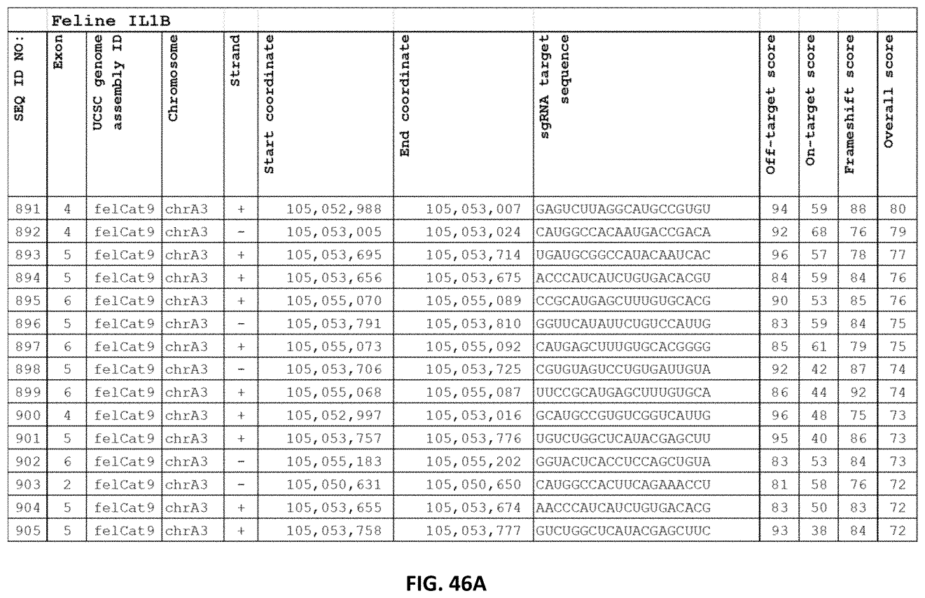

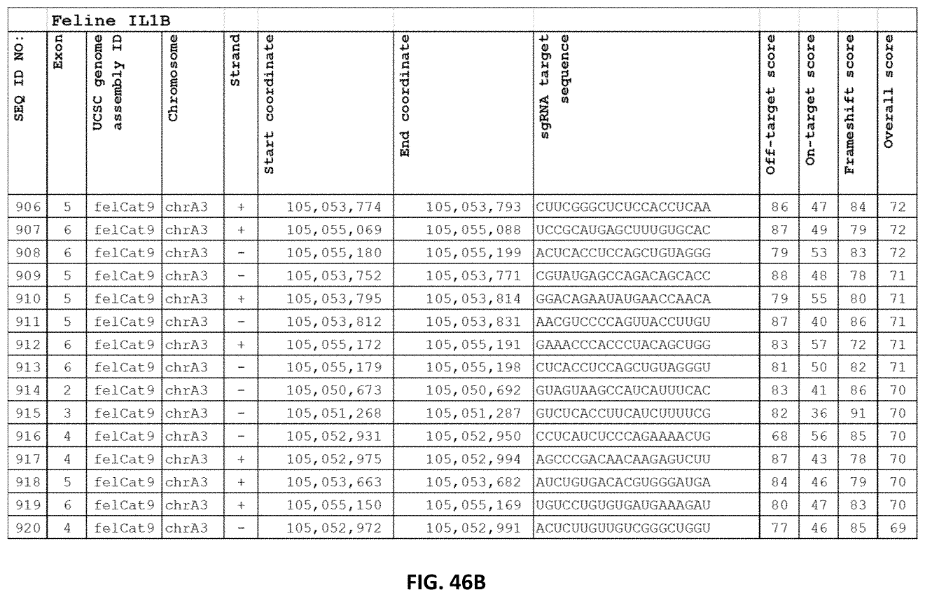

(SEQ ID NO: 682, left column)
cagagacagatgatcaatgg,
(SEQ ID NO: 1124, right column)
ATTCAGAGACAGATGATCAAATGGAGGAACT;
#236:
(SEQ ID NO: 636, left column)
gccatagcttacatgataga,
(SEQ ID NO: 1125, right column)
TGGGCCATAGCTTACATGATTAGAAGGATTT;
#237:
(SEQ ID NO: 498, left column)
tgatggccctggaaatgtga,
(SEQ ID NO: 1126, right column)
AGCTGATGGCCCTAAACAGAATGAAGGTAAG;
#238
(SEQ ID NO: 391, left column)
ggtggtcggagattcgtagc
(SEQ ID NO: 1127, right column
AGTGGTGGTCGGAGATTCGTAGCTGGATGCCGC;
#248
(SEQ ID NO: 190, left column)
acctatcttcttcgacacat,
(SEQ ID NO: 1128, right column
AGTGGTGGTCGGAGATTCGTAGCTGGATGCCGC;
#249
(SEQ ID NO: 60, left column)
cttcgacacatgggataacg,
(SEQ ID NO: 1129, right column)
CTTCTTCGACACATGGGATAAAGAAGGCTTA;
#250
(SEQ ID NO: 61)
gtgcagttcagtgatcgtac,
(SEQ ID NO: 1130, right column)
AGCGTGCAGTTCAGTGATCTACAGGTGCATCG;
#259
(SEQ ID NO: 390, left column)
catggccacaacaactgacg,
(SEQ ID NO: 1130, right column)
GTCCATGGCCACAACAACTACGCGGCCTGCCT;
#260
(SEQ ID NO: 440, left column)
aggtcctggaaggagcactg,
(SEQ ID NO: 1132)
TCCAGGTCCTGGAAGGAGCAACTGCGGAGAG.
For FIG. 28B,
numbers #239:
(SEQ ID NO: 552, left column)
tgaccatctctctctgaatc,
(SEQ ID NO: 1133, right column)
AATTGACCATCTCTCTCTGAAATCAGGTAAG;
#240:
(SEQ ID NO: 554, left column)
gacatcccagcttaccttca,
(SEQ ID NO: 1134, right column)
AAAGACATCCCAGCTTACCTTTCAAGGAAAA;
#251:
(SEQ ID NO: 578, left column)
agtatagttcgacaaacagg,
(SEQ ID NO: 1135, right column)
CAAAGTATAGTTCGACAAACCAGGAGGAAAT;
#252
(SEQ ID NO: 579, left column)
tctgtaatgcagcagtcatg,
(SEQ ID NO: 1136, right column)
AATTCTGTAATGCAGCAGTCCATCAGGTAAT;
#241
(SEQ ID NO: 781, left column)
tgatggccctggaaatgtga,
(SEQ ID NO: 1137, right column)
AGCTGATGGCCCTGGAAATGAAGGTGAGACC;
#242
(SEQ ID NO: 875, left column)
actcttcttgagggtaagct,
(SEQ ID NO: 1138, right column)
CAGATCCTTGTTACAGAGCTTGGTGGGAGAC.

FIG. 29 illustrates results of co-administrating lead candidate sgRNAs in canine cells either simultaneously or sequentially.

FIGS. 30A and 30 B illustrate sequence alignments of IL1A (A) and IL1B (B) genomic sequences from human, equine, murine, and canine for potential cross-species targets with a single sgRNA. The sequences are: #235

(SEQ ID NO: 1094)
GTTCCTCCATTGATCATCTGTCTCTGAAT;
#236
(SEQ ID NO: 1095)
AATCCTTCTATCATGTAAGCTATGGCCCA;
#239
(SEQ ID NO: 1096)
AATTGACCATCTCTCTCTGAATCAGGTAA;
#240
(SEQ ID NO: 1097)
AAAGACATCCCAGCTTACCTTCAAGGAAA;
#251
(SEQ ID NO: 1098)
CAAAGTATAGTTCGACAAACAGGAGGAAA;
#252
(SEQ ID NO: 1099)
TTACCTCATGACTGCTGCATTACAGAATT;
#086
(SEQ ID NO: 1100)
ACAGTATCAGCAACGTCAAGCAACGGGAA;
#087
(SEQ ID NO: 1101)
CAGTATCAGCAACGTCAAGCAACGGGAAG;
#096
(SEQ ID NO: 1102)
AGACCTTCACTGAAGATGACCTGCAGTCC;
#101
(SEQ ID NO: 1103)
AATCCTTCTATGATGCAAGCTATGGCTCA;
#237
(SEQ ID NO: 1104)
AGCTGATGGCCCTAAACAGATGAAGGTAA;
#238
(SEQ ID NO: 1105)
CATCCAGCTACGAATCTCCGACCACCACT;
#248
(SEQ ID NO: 1106)
AGAACCTATCTTCTTCGACACATGGGATA;
#249
(SEQ ID NO: 1107)
CTTCTTCGACACATGGGATAACGAGGCTT;
#250
(SEQ ID NO: 1108)
GCACCTGTACGATCACTGAACTGCACGCT;
#259
(SEQ ID NO: 1109)
AGGCCGCGTCAGTTGTTGTGGCCATGGAC;
#260
(SEQ ID NO: 1110)
TCTCCGCAGTGCTCCTTCCAGGACCTGGA;
#241
(SEQ ID NO: 1111)
AGCTGATGGCCCTGGAAATGTGAAGGTGA;
#242
(SEQ ID NO: 1112)
TCTCCCACCAGCTCTGTAACAAGAGTCTG;
#063
(SEQ ID NO: 1113)
GCACATCAACAAGAGCTTCAGGCAGGCAG;
#074
(SEQ ID NO: 1114)
GGACCTTCCAGGATGAGGACATGAGCACC;
#080
(SEQ ID NO: 1115)
TGACCTGGGCTGTCCTGATGAGAGCATCC.

FIGS. 31A, 31B, and 31C collectively illustrate CRISPR-mediated editing of canine IL-1A. Specifically, FIG. 31A shows mean KO scores determined by inference of CRISPR edits from Sanger traces in canine chondrocytes (n=3+), fibroblast-like synoviocytes (n=3) and monocytes DH82 (n=2). Stacked bar graph shows the top and all remaining KO genotypes of sgRNAs assembled with wild-type Cas9 in light and dark, respectively. FIG. 31B shows the amino acid sequence of wild-type and truncated canine IL1A prior to (top) and after (bottom) CRISPR-mediated genome editing. Wild-type IL1A includes a propeptide, which is cleaved off to make functional (mature) protein (highlighted). FIG. 31C illustrates the predicted three-dimensional structure of wild-type and truncated IL1A, as predicted by AlphaFold2. N marks the N-terminus, the canonical translational start.

FIGS. 32A, 32B, and 32C collectively illustrate design and testing of eight canine IL1A-targeting sgRNAs: sg239/OCA01 (SEQ ID NO:552), sg360/OCA02 (SEQ ID NO:564), sg240/OCA03 (SEQ ID NO:554), sg359/OCA04 (SEQ ID NO:746), sg358/OCA05 (SEQ ID NO:741), sg251/OCA07 (SEQ ID NO:743), sg361/OCA06 (SEQ ID NO:748), and sg252/OCA08 (SEQ ID NO:752). FIG. 32A shows the target sequence edited by the guide (equivalent to the crRNA sequence in the guide, except that the guide sequence includes uracil in place pf thymidine). FIG. 32B shows scores for predicted on-target, off-target, and frameshift effects for each of the gRNA, as described in Example 16. Sequences are shown as DHLSLN (SEQ ID NO:1116); MSCDPL (SEQ ID NO:1117); TSQLTF (SEQ ID NO: 1118); IMKPRS (SEQ ID NO: 1119; LNDNLN (SEQ ID NO: 1120); QSIVRQ (SEQ ID NO:1121); SIVRQT (SEQ ID NO:1122); GGNYLM (SEQ ID NO:1123). FIG. 32C shows results of in vitro editing assays analyzed by sequencing of edited targets.

FIGS. 33A, 33B, 33C, and 33D collectively illustrate design and testing of eight canine IL1B-targeting sgRNAs: sg241/OCB01 (SEQ ID NO: 498), sg242/OCB02 (SEQ ID NO: 506), sg352/OCB06 (SEQ ID NO: 771), sg353/OCB04 (SEQ ID NO: 531), sg354/OCB08 (SEQ ID NO: 773), sg355/OCB05 (SEQ ID NO: 778), sg356/OCB07 (SEQ ID NO: 539), and sg357/OCB03 (SEQ ID NO: 669). FIG. 33A shows the target sequence edited by the guide (equivalent to the crRNA sequence in the guide, except that the guide sequence includes uracil in place pf thymidine); #353 (SEQ ID NO:772), #356 (SEQ ID NO:539), #352 (SEQ ID NO:771), #354 (SEQ ID NO:535), #355 (SEQ ID NO:538), #357 (SEQ ID NO:956), #242 (SEQ ID NO:920), and #241 (SEQ ID NO:462) and the amino acid cut positions: #353 MSDAAM-Q (SEQ ID NO:1139); #356 ELRALH-L (SEQ ID NO:1140); #352 SDAAMQ-S(SEQ ID NO:1141); #354 LRALHL-N(SEQ ID NO:1142); #355 MSDAAM-Q (SEQ ID NO:1139); #357 LQVSHQ-L (SEQ ID NO:1144); #242 QLQVSH-Q (SEQ ID NO:1143); #241 ADGPGN-V (SEQ ID NO:1145). FIG. 33B shows scores for predicted on-target, off-target, and frameshift effects for each of the gRNA, as described in Example 17, and the amino acid cut positions: #353 MSDAAM-Q (SEQ ID NO:1139); #356 ELRALH-L (SEQ ID NO:1140); #352 SDAAMQ-S(SEQ ID NO: 1141); #354 LRALHL-N (SEQ ID NO:1142); #355 MSDAAM-Q (SEQ ID NO:1139); #357 LQVSHQ-L (SEQ ID NO:1144); #242 QLQVSH-Q (SEQ ID NO:1143); #241 ADGPGN-V (SEQ ID NO:1145). FIG. 33C shows results of in vitro editing assays analyzed by sequencing of edited targets. FIG. 33D shows results of anti-IL-1B ELISA assays on the supernatant of canine monocytes targeted by the editing constructs.

FIGS. 34A and 34B collectively illustrate performance of IL1B-targeting sgRNA #241 (OCB01) and #242 (OCB02), as measured by mean IL1B KO scores inferred from Sanger-sequencing CRISPR-mediated genomic edits in chondrocytes, synoviocytes and monocytes DH82 (FIG. 34A) and ELISA results for IL1B in either control or two IL1A/B double KOs monocytes generated from different combinations of sgRNAs (FIG. 34B; Sanger sequence-inferred KO scores for each combination at left).

FIGS. 35A and 35B collectively illustrate the impact of electroporation on canine monocytes with either OCB02/sg242 (KO1), or OCB01/sg241 (KO2), as measured by supernatant ELISA at either (A) 6 and (B) 24 hours following challenge with lipopolysaccharide (LPS).

FIG. 36 illustrates a comparison of editing efficiencies for 5 potential gRNAs targeting canine IL-1 beta in monocytes, as measured by Abcam IL-1 beta ELISA kit.

FIGS. 37A and 37 B collectively illustrate the effect of different Cas9 mutants with enhanced specificity, including (A) a comparison of on-target editing efficiency with the OCB02 gRNA plus either standard Cas9 or enhanced specificity/fidelity Cas9 variants and (B) a comparison of the on-target editing efficiency of OCB01 with the ARCas9 variant or wild-type Cas9.

FIGS. 38A and 38B collectively illustrate the in vitro performance of Cas9 mutants with enhanced specificity. FIG. 38A illustrates editing efficiency for on-targets and off-targets, where percentages represent the KO scores and editing efficiencies, respectively. FIG. 38B shows predicted off-target editing sites for OCB01; genomic loci

(SEQ ID NO: 1146)
ACTCTTGTTACAGAGCTGGT,
(SEQ ID NO: 1147)
ACTCTTGTTACAGAGCTGGTNGG,
(SEQ ID NO: 1148)
ACTCTTGTTACAGAGCTGGTNGA,
and
(SEQ ID NO: 1149)
ACTCTTGTTACAGAGCTGGTNAG.

FIGS. 39A and 39B collectively illustrate example CRISPR/Cas9 crRNA sequences designed for editing the human IL-1 alpha gene.

FIGS. 40A and 40B collectively illustrate example CRISPR/Cas9 crRNA sequences designed for editing the human IL-1 beta gene.

FIGS. 41A and 41B collectively illustrate example CRISPR/Cas9 crRNA sequences designed for editing the canine IL-1 alpha gene.

FIGS. 42A and 42B collectively illustrate example CRISPR/Cas9 crRNA sequences designed for editing the canine IL-1 beta gene.

FIGS. 43A and 43B collectively illustrate example CRISPR/Cas9 crRNA sequences designed for editing the equine IL-1 alpha gene.

FIGS. 44A and 44B collectively illustrate example CRISPR/Cas9 crRNA sequences designed for editing the equine IL-1 beta gene.

FIGS. 45A and 45B collectively illustrate example CRISPR/Cas9 crRNA sequences designed for editing the feline IL-1 alpha gene.

FIGS. 46A and 46B collectively illustrate example CRISPR/Cas9 crRNA sequences designed for editing the feline IL-1 beta gene.

While the above-identified drawing sets forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

As described herein, embodiments of the present disclosure provide compositions and methods for improving joint function and treating joint disease. In particular embodiments, compositions and methods are provided to gene-edit synovial fibroblasts, synoviocytes, chondrocytes, or synovial macrophages to reduce expression of inflammatory cytokines, for example, IL-1α, IL-1β, TNF-α, IL-6, IL-8, IL-18, one or more matrix metalloproteinases (MMPs), or one or more component of the NLRP3 inflammasome. Embodiments are used for treating osteoarthritis and other inflammatory joint diseases. Embodiments are further useful for treating canine lameness due to osteoarthritis. Embodiments are further useful for treating equine lameness due to joint disease. Embodiments are also useful for treating post-traumatic arthritis, gout, pseudogout, and other inflammation-mediated or immune-mediated joint diseases.

### Definitions

Unless defined otherwise, all technical and scientific terms used herein have the same meaning as is commonly understood by one of skill in the art to which this disclosure belongs. All patents and publications referred to herein are incorporated by reference in their entireties.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “ex vivo” refers to an event which involves treating or performing a procedure on a cell, tissue and/or organ which has been removed from a subject's body. Aptly, the cell, tissue and/or organ may be returned to the subject's body in a method of surgery or treatment.

The term “IL-1” (also referred to herein as “IL1”) refers to the pro-inflammatory cytokine known as interleukin-1, and includes all forms of IL-1, including IL1-α and IL-1β, human and mammalian forms, conservative amino acid substitutions, glycoforms, biosimilars, and variants thereof. IL-1α and IL-1β bind to the same receptor molecule, which is called type I IL-1 receptor (IL-1R1). There is a third ligand of this receptor: Interleukin 1 receptor antagonist (IL-1Ra), which does not activate downstream signaling; therefore, acting as an inhibitor of IL-1α and IL-1β signaling by competing with them for binding sites of the receptor. See, e.g., Dinarello, Blood 117: 3720-32 (2011) and Weber et al., Science Signaling 3(105): cm1, doi:10.1126/scisignal.3105cm1. IL-1 is described, e.g., in Dinarello, Cytokine Growth Factor Rev. 8:253-65 (1997), the disclosures of which are incorporated by reference herein. For example, the term IL-1 encompasses human, recombinant forms of IL-1.

TABLE 1
Example amino acid sequences.

| Identifier | Sequence (One-Letter Amino Acid Symbols) |
|---|---|
| SEQ ID NO: 1 | 10 20 30 40 50 |
| recombinant | MAKVPDMFED LKNCYSENEE DSSSIDHLSL NQKSFYHVSY GPLHEGCMDQ |
| human IL-1alpha | 60 70 80 90 100 |
| (rhIL-1α) | SVSLSISETS KTSKLTFKES MVVVATNGKV LKKRRLSLSQ SITDDDLEAI |
| | 110 120 130 140 150 |
| | ANDSEEEIIK PRSAPFSFLS NVKYNFMRII KYEFILNDAL NQSIIRANDQ |
| | 160 170 180 190 200 |
| | YLTAAALHNL DEAVKFDMGA YKSSKDDAKI TVILRISKTQ LYVTAQDEDQ |
| | 210 220 230 240 250 |
| | PVLLKEMPEI PKTITGSETN LLFFWETHGT KNYFTSVAHP NLFIATKQDY |
| | 260 270 |
| | WVCLAGGPPS ITDFQILENQ A |
| SEQ ID NO: 2 | 10 20 30 40 50 |
| recombinant | MAEVPELASE MMAYYSGNED DLFFEADGPK QMKCSFQDLD LCPLDGGIQL |
| human IL-1beta | 60 70 80 90 100 |
| (rhIL-1β) | RISDHHYSKG FRQAASVVVA MDKLRKMLVP CPQTFQENDL STFFPFIFEE |
| | 110 120 130 140 150 |
| | EPIFFDTWDN EAYVHDAPVR SLNCTLRDSQ QKSLVMSGPY ELKALHLQGQ |
| | 160 170 180 190 200 |
| | DMEQQVVFSM SFVQGEESND KIPVALGLKE KNLYLSCVLK DDKPTLQLES |
| | 210 220 230 240 250 |
| | VDPKNYPKKK MEKRFVFNKI EINNKLEFES AQFPNWYIST SQAENMPVFL |
| | 260 |
| | GGTKGGQDIT DFTMQFVSS |
| SEQ ID NO: 3 | 10 20 30 40 50 |
| recombinant | MAKVPDLFED LKNCYSENED YSSAIDHLSL NQKSFYDASY GSLHETCTDQ |
| mouse IL-1alpha | 60 70 80 90 100 |
| (rmIL-1α) | FVSLRTSETS KMSNFTFKES RVTVSATSSN GKILKKRRLS FSETFTEDDL |
| | 110 120 130 140 150 |
| | QSITHDLEET IQPRSAPYTY QSDLRYKLMK LVRQKFVMND SLNQTIYQDV |
| | 160 170 180 190 200 |
| | DKHYLSTTWL NDLQQEVKFD MYAYSSGGDD SKYPVTLKIS DSQLFVSAQG |
| | 210 220 230 240 250 |
| | EDQPVLLKEL PETPKLITGS ETDLIFFWKS INSKNYFTSA AYPELFIATK |
| | 260 270 |
| | EQSRVHLARG LPSMTDFQIS |
| SEQ ID NO: 4 | 10 20 30 40 50 |
| recombinant | MATVPELNCE MPPFDSDEND LFFEVDGPQK MKGCFQTFDL GCPDESIQLQ |
| mouse IL-1beta | 60 70 80 90 100 |
| (rmIL-1β) | ISQQHINKSF RQAVSLIVAV EKLWQLPVSF PWTFQDEDMS TFFSFIFEEE |
| | 110 120 130 140 150 |
| | PILCDSWDDD DNLLVCDVPI RQLHYRLRDE QQKSLVLSDP YELKALHLNG |
| | 160 170 180 190 200 |
| | QNINQQVIFS MSFVQGEPSN DKIPVALGLK GKNLYLSCVM KDGTPTLQLE |
| | 210 220 230 240 250 |
| | SVDPKQYPKK KMEKRFVFNK IEVKSKVEFE SAEFPNWYIS TSQAEHKPVF |
| | 260 |
| | LGNNSGQDII DFTMESVSS |
| SEQ ID NO: 5 | 10 20 30 40 50 |
| recombinant | MEICRGLRSH LITLLLFLFH SETICRPSGR KSSKMQAFRI WDVNQKTFYL |
| human IL-1 | 60 70 80 90 100 |
| receptor | RNNQLVAGYL QGPNVNLEEK IDVVPIEPHA LFLGIHGGKM CLSCVKSGDE |
| antagonist | 110 120 130 140 150 |
| (rhIL-1Ra) | TRLQLEAVNI TDLSENRKQD KRFAFIRSDS GPTTSFESAA CPGWFLCTAM |
| | 160 170 |
| | EADQPVSLTN MPDEGVMVTK FYFQEDE |
| SEQ ID NO: 6 | 10 20 30 40 50 |
| recombinant | MEICWGPYSH LISLLLILLF HSEAACRPSG KRPCKMQAFR IWDTNQKTFY |
| mouse IL-1 | 60 70 80 90 100 |
| receptor | LRNNQLIAGY LQGPNIKLEE KIDMVPIDLH SVFLGIHGGK LCLSCAKSGD |
| antagonist | 110 120 130 140 150 |
| (rmIL-1Ra) | DIKLQLEEVN ITDLSKNKEE DKRFTFIRSE KGPTTSFESA ACPGWFLCTT |
| | 160 170 |
| | LEADRPVSLT NTPEEPLIVT KFYFQEDQ |

The term “NLRP3 inflammasome” refers to the multiprotein complex responsible for the activation of some inflammatory responses. The NLRP3 inflammasome promotes the production of functional pro-inflammatory cytokines, for example, IL-1β and IL-18. Core components of the NLRP3 inflammasome are NLRP3, ASC (apoptosis-associated speck-like protein containing a CARD), and caspase-1, as described by Lee et al., Lipids Health Dis. 16:271 (2017) and Groslambert and Py, J. Inflamm. Res. 11:359-374 (2018).

The terms “matrix metalloproteinase” and “MMP” are defined to be any one of the members of the matrix metalloproteinase family of zinc-endopeptidaes, for example, as characterized by Fanjul-Femandez et al., Biochem. Biophys. Acta 1803:3-19 (2010). In the art, family members are frequently referred to as archetypical MMPs, gelatinases, matrilysins, and/or furin-activatable MMPs. As used herein, the “matrix metalloproteinase” and “MMP” encompass the entire MMP family, including, but not limited to MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-23, MMP-25, MMP-26, MMP-27 and MMP-28.

The terms “co-administration,” “co-administering,” “administered in combination with,” “administering in combination with,” “simultaneous,” and “concurrent,” as used herein, encompass administration of two or more active pharmaceutical ingredients (in a preferred embodiment of the present disclosure, for example, at least one anti-inflammatory compound in combination with a viral vector functionally engineered to deliver a gene-editing nucleic acid as described herein) to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “effective amount” or “therapeutically effective amount” refers to that amount of a composition or combination of compositions as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compositions chosen, the dosing regimen to be followed, whether the composition is administered in combination with other compositions or compounds, timing of administration, the tissue to which it is administered, and the physical delivery system in which the composition is carried.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. For example, a composition, method, or system of the present disclosure may be administered as a prophylactic treatment to a subject that has a predisposition for a given condition (e.g., arthritis). “Treatment”, as used herein, covers any treatment of a disease in a mammal, particularly in a human, canine, feline, or equine, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine. It is understood that compositions and methods of the present disclosure are applicable to treat all mammals, including, but not limited to human, canine, feline, equine, and bovine subjects.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The terms “polynucleotide,” “nucleotide,” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, rRNA, tRNA, lncRNA, RNA antagomirs, and inhibitory DNA or RNA (RNAi, e.g., small or short hairpin (sh)RNA, microRNA (miRNA), aptamers, small or short interfering (si)RNA, trans-splicing RNA, or antisense RNA). Polynucleotides also include non-coding RNA, which include for example, but are not limited to, RNAi, miRNAs, lncRNAs, RNA antagomirs, aptamers, and any other non-coding RNAs known to those of skill in the art. Polynucleotides include naturally occurring, synthetic, and intentionally altered or modified polynucleotides as well as analogues and derivatives. The term “polynucleotide” also refers to a polymeric form of nucleotides of any length, including deoxyribonucleotides or ribonucleotides, or analogs thereof, and is synonymous with nucleic acid sequence. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment as described herein encompassing a polynucleotide encompasses both the double-stranded form and each of two complementary single-stranded forms known or predicted to make up the double-stranded form. Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

The term “gene” or “nucleotide sequence encoding a polypeptide” refers to the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons). For example, a gene includes a polynucleotide containing at least one open reading frame capable of encoding a particular protein or polypeptide after being transcribed and translated.

The term “homologous” in terms of a nucleotide sequence includes a nucleotide (nucleic acid) sequence that is either identical or substantially similar to a known reference sequence. In one embodiment, the term “homologous nucleotide sequence” is used to characterize a sequence having nucleic acid sequence that is at least 70%, at least 75%, at least 80%, at least 85%, at least 90%, at least 95%, at least 96%, at least 97%, at least 98%, at least 99%, or 100% identical to a known reference sequence.

“Heterologous” means derived from a genotypically distinct entity from the rest of the entity to which it is being compared to. For example, a polynucleotide introduced by genetic engineering techniques into a plasmid or vector derived from a different species is a heterologous polynucleotide. A promoter removed from its native coding sequence and operatively linked to a coding sequence it is not naturally found linked to a heterologous promoter. Although the term “heterologous” is not always used herein in reference to polynucleotides, reference to a polynucleotide even in the absence of the modifier “heterologous” is intended to include heterologous polynucleotides in spite of the omission.

The terms “sequence identity,” “percent identity,” and “sequence percent identity” (or synonyms thereof, e.g., “99% identical”) in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. ClustalW and ClustalX may be used to produce alignments, Larkin et al., Bioinformatics 23:2947-2948 (2007); Goujon et al., Nucleic Acids Research, 38 Suppl:W 695-9 (2010); and, McWilliam et al., Nucleic Acids Research 41 (Web Server issue):W 597-600 (2013). One skilled in the art can determine appropriate parameters for maximal alignment by particular alignment software. In certain embodiments, the default parameters of the alignment software are used.

As used herein, the term “variant” encompasses but is not limited to antibodies or fusion proteins which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody. The term variant also includes pegylated antibodies or proteins.

“Joint disease” is defined as measurable abnormalities in the cells or tissues of the joint that could lead to illness, for example, metabolic and molecular derangements triggering anatomical and/or physiological changes in the joint. Including, but not limited to, radiographic detection of joint space narrowing, subchondral sclerosis, subchondral cysts, and osteophyte formation.

“Joint illness” is defined in human subjects as symptoms that drive the subject to seek medical intervention, for example, subject reported pain, stiffness, swelling, or immobility. For non-human mammals, “joint illness” is defined, for example, as lameness, observable changes in gait, weight bearing, allodynia, or exploratory behavior.

As used herein, a sgRNA (single guide RNA) is a RNA, preferably a synthetic RNA, composed of a targeting sequence and scaffold. It is used to guide Cas9 to a specific genomic locus in genome engineering experiments. The sgRNA can be administered or formulated, e.g., as a synthetic RNA, or as a nucleic acid comprising a sequence encoding the gRNA, which is then expressed in the target cells. As would be evident to one of ordinary skill in the art, various tools may be used to design and/or optimize the sequence of a sgRNA, for example to increase the specificity and/or precision of genomic editing. In general, candidate sgRNAs may be designed by identifying a sequence within the target region that has a high predicted on-target efficiency and low off-target efficiency based on any of the available web-based tools. Candidate sgRNAs may be further assessed by manual inspection and/or experimental screening. Examples of web-based tools include, without limitation, CRISPR seek, CRISPR Design Tool, Cas-OFFinder, E-CRISP, ChopChop, CasOT, CRISPR direct, CRISPOR, BREAKING-CAS, CrispRGold, and CCTop. See, e.g., Safari, et al. Current Pharma. Biotechol. (2017) 18(13), which is incorporated by reference herein in its entirety for all purposes. Such tools are also described, for example, in PCT Publication No. WO2014093701A1 and Liu, et al., “Computational approached for effective CRISPR guide RNA design and evaluation”, Comput Struct Biotechnol J., 2020; 18: 35-44, each of which is incorporated by reference herein in its entirety for all purposes.

As used herein, “Cas9” refers to CRISPR Associated Protein; the Cas9 nuclease is the active enzyme for the Type II CRISPR system. “nCas9” refers to a Cas9 that has one of the two nuclease domains inactivated, i.e., either the RuvC or HNH domain. nCas9 is capable of cleaving only one strand of target DNA (a “nickase”). The term “Cas9” refers to an RNA-guided double-stranded DNA-binding nuclease protein or nickase protein, or a variant thereof. Herein, “Cas9” refers to both naturally-occurring and recombinant Cas9s. Wild-type Cas9 nuclease has two functional domains, e.g., RuvC and HNH, that cut different DNA strands. Cas9 enzymes described herein can comprise a HNH or HNH-like nuclease domain and/or a RuvC or RuvC-like nuclease domain. Cas9 can induce double-strand breaks in genomic DNA (target locus) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species.

As used herein, “PAM” refers to a Protospacer Adjacent Motif and is necessary for Cas9 to bind target DNA, and immediately follows the target sequence. The Cas9 can be administered or formulated, e.g., as a protein (e.g., a recombinant protein), or as a nucleic acid comprising a sequence encoding the Cas9 protein, which is then expressed in the target cells. Naturally occurring Cas9 molecules recognize specific PAM sequences (e.g., the PAM recognition sequences for S. pyogenes, S. thermophilus, S. mutans, S. aureus and N. meningitidis). In an embodiment, a Cas9 molecule has the same PAM specificities as a naturally occurring Cas9 molecule. In other embodiments, a Cas9 molecule has a PAM specificity not associated with a naturally occurring Cas9 molecule. In other embodiments, a Cas9 molecule's PAM specificity is not associated with the naturally occurring Cas9 molecule to which it has the closest sequence homology. For example, a naturally occurring Cas9 molecule can be altered such that the PAM sequence recognition is altered to decrease off target sites, improve specificity, or eliminate a PAM recognition requirement. In an embodiment, a Cas9 molecule may be altered (e.g., to lengthen a PAM recognition sequence, improve Cas9 specificity to high level of identity, to decrease off target sites, and/or increase specificity). In an embodiment, the length of the PAM recognition sequence is at least 4, 5, 6, 7, 8, 9, 10 or 15 amino acids in length. In some embodiments, a Cas9 molecule may be altered to ablate PAM recognition.

An “expression cassette” is a nucleic acid construct, generated recombinantly or synthetically, with a series of specified nucleic acid elements that permit transcription of a particular polynucleotide sequence in a host cell. An expression cassette or vector may be part of a plasmid, viral genome, or nucleic acid fragment. Typically, an expression cassette or vector includes a polynucleotide to be transcribed, operably linked to a promoter.

The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators), as well as those that confer certain binding affinity or antigenicity to the recombinant protein produced from the expression vector.

The term “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

An “isolated” plasmid, nucleic acid, vector, virus, virion, host cell, or other substance refers to a preparation of the substance devoid of at least some of the other components present where the substance or a similar substance naturally occurs or from which it is initially prepared. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this disclosure are increasingly more isolated. An isolated plasmid, nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

An “AAV vector” as used herein refers to an AAV vector nucleic acid sequence encoding for various nucleic acid sequences, including in some embodiments a variant or chimeric capsid polypeptide (i.e., the AAV vector comprises a nucleic acid sequence encoding for a variant or chimeric capsid polypeptide). AAV vectors can also comprise a heterologous nucleic acid sequence not of AAV origin as part of the nucleic acid insert. This heterologous nucleic acid sequence typically comprises a sequence of interest for the genetic transformation of a cell. In general, the heterologous nucleic acid sequence is flanked by at least one, and generally by two AAV inverted terminal repeat sequences (ITRs). In certain embodiments, a Cas sequence, a guide RNA sequence, and any other genetic element (e.g., a promoter sequence, a PAM sequence, and the like) may be on the same AAV vector or on two or more different AAV vectors when administered to a subject. In certain embodiments, a Cas sequence, a guide RNA sequence, and any other genetic element (e.g., a promoter sequence, a PAM sequence, and the like) may be on two or more different AAV vectors when administered to a subject, and the AAV may be the same serotype, or the AAV may be two or more different serotypes (e.g., AAV5 and AAV6).

An “AAV virion” or “AAV virus” or “AAV viral particle” or “AAV vector particle” refers to a viral particle composed of at least one AAV capsid polypeptide and an encapsidated polynucleotide AAV transfer vector. If the particle comprises a heterologous nucleic acid (i.e. a polynucleotide other than a wild-type AAV genome, such as a transgene to be delivered to a cell), it can be referred to as an “AAV vector particle” or simply an “AAV vector”. Thus, production of AAV virion or AAV particle necessarily includes production of AAV vector as such a vector is contained within an AAV virion or AAV particle.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, or the like, which is nontoxic and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

The term “pharmaceutically acceptable excipient” is intended to include vehicles and carriers capable of being co-administered with a compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present disclosure.

As used herein, the term “a”, “an”, or “the” generally is construed to cover both the singular and the plural forms.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that compositions, amounts, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The term “substantially” as used herein can refer to a majority of, or mostly, as in at least about 50%, 60%, 70%, 80%, 90%, 95%, 96%, 97%, 98%, 99%, 99.5%, 99.9%, 99.99%, or at least about 99.999% or more.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed methods and compositions. All compositions, methods, and kits described herein that embody the present disclosure can, in alternate embodiments, be more specifically defined by any of the transitional terms “comprising,” “consisting essentially of,” and “consisting of.”

A subject treated by any of the methods or compositions described herein can be of any age and can be an adult, infant or child. In some cases, the subject is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., without limitation, between 2 and 20 years old, between 20 and 40 years old, or between 40 and 90 years old). The subject can be a human or non-human subject. A particular class of subjects that can benefit from the compositions and methods of the present disclosure include subjects over the age of 40, 50, or 60 years. Another class of subjects that can benefit from the compositions and methods of the present disclosure are subjects that have arthritis (e.g., osteoarthritis).

Any of the compositions disclosed herein can be administered to a non-human subject, such as a laboratory or farm animal. Non-limiting examples of a non-human subject include laboratory or research animals, pets, wild or domestic animals, farm animals, etc., e.g., a dog, a goat, a guinea pig, a hamster, a mouse, a pig, a non-human primate (e.g., a gorilla, an ape, an orangutan, a lemur, a baboon, etc.), a rat, a sheep, a horse, a cow, or the like.

CRISPR/Cas Systems—Minimum Requirements

In one aspect, clustered regularly interspaced short palindromic repeats and CRISPR-associated RNA-guided nuclease-related methods, components and compositions of the disclosure (hereafter, CRISPR/Cas systems) minimally require at least one isolated or non-naturally-occurring protein component (e.g., a Cas protein) and at least one isolated or non-naturally-occurring nucleic acid component (e.g., a guide RNA (gRNA)) to effectuate augmentation of a ‘nucleic acid sequence (e.g., genomic DNA).

In some embodiments, a CRISPR/Cas system effectuates the alteration of a targeted gene or locus in a eukaryotic cell by effecting an alteration of the sequence at a target position (e.g., by creating an insertion or deletion (collectively, an indel) resulting in loss-of-function of (i.e., knocking out) the affected gene or allele; e.g., a nucleotide substitution resulting in a truncation, nonsense mutation, or other type of loss-of-function of an encoded gene product of, for example, the encoded IL1A or IL1B mRNA or protein; a deletion of one or more nucleotides resulting in a truncation, nonsense mutation, or other type of loss-of-function of, for example, an encoded IL1A or IL1B gene product; e.g., loss-of-function of the encoded mRNA or protein by a single nucleotide, double nucleotide, or other frame-shifting deletion, or a deletion resulting in a premature stop codon; or an insertion resulting in a truncation, nonsense mutation, or other type of loss-of-function of an encoded gene product, such as, for example, the encoded IL1A or IL1B mRNA or protein; e.g., a single nucleotide, double nucleotide, or other frame-shifting insertions, or an insertion resulting in a premature stop codon. In some embodiments, a CRISPR/Cas system of the present disclosure provides for the alteration (e.g., knocking out) of a gene associated with inflammatory joint diseases (e.g., rheumatoid arthritis or osteoarthritis) by altering the sequence at a target position, e.g., creating an indel that results in nonsense-mediated decay of an encoded gene product, e.g., an encoded transcript.

In one aspect, CRISPR/Cas systems effectuate changes to the sequence of a nucleic acid through nuclease activity. For example, in the case of genomic DNA, the nuclease-guided by a protein-associated exogenous nucleic acid that locates a target position within a targeted gene or locus by sequence complementarity with the target genomic sequence (e.g., CRISPR RNA (crRNA) or a complementary component of a synthetic single guide RNA (sgRNA))—cleaves the genomic DNA upon recognition of particular, nuclease-specific motif called the protospacer adjacent motif (PAM). See generally, Collias, D., & Beisel, C. L. (2021). Nature Communications, 12(1), 1-12.

Nuclease activity (i.e., cleavage) induces a double-strand break (DSB) in the case of genomic DNA. Endogenous cellular mechanisms of DSB repair, namely non-homologous end joining (NHEJ), microhomology-mediated end joining (MMEJ), and homologous recombination, result in erroneous repair at a given target position with some calculable frequency as a result of interference from said components of the CRISPR/Cas system, thereby introducing substitutions or indels into the genomic DNA. See generally Scully, R., et al. (2019). Nature Reviews Molecular Cell Biology, 20(11), 698-714. At some frequency, these indels and/or substitutions may result in frameshifts, nonsense mutations (i.e., early stop codons) or truncations that impact the availability of gene products, such as mRNA and/or protein. In certain embodiments, the CRISPR/Cas system may induce a homology-directed repair (HDR) mechanism leading to insertions of non-random sequences at a target position through the use of templates (e.g., an HDR template) provided to the cell as part of the system along with the nuclease and gRNA. See Bloh, K., & Rivera-Torres, N. (2021). International Journal of Molecular Sciences, 22(8), 3834.

In general, the minimum requirements of the CRISPR/Cas system will be dependent upon the nuclease (i.e., Cas protein) provided therewith. To this extent, these bacterially-derived nucleases have been functionally divided into Types I, III, and V, which all fall into Class 1 and Types II, IV, and VI that are grouped into Class 2.

Class 1 CRISPR/Cas Systems:

The exact components, compositions, and methods for effectuating a change in a targeted nucleic acid sequence using a Class 1 CRISPR/Cas system will vary, but should minimally include: a nuclease (selected from at least Types I, and III), at least one guide RNA selected from 1) sgRNA or 2) a combination of crRNA and tracrRNA. These CRISPR/Cas systems have been categorized together as Class 1 CRISPR/Cas systems due to their similarities in requirements and mode of action within a eukaryotic cell. To this end, compositions, components, and methods among Class 1 constituents may be considered functionally interchangeable, and the following details, provided merely for exemplary purposes, do not represent an exhaustive list of class members:

Cas3 (see Table 2) is the prototypical Type I DNA nuclease that functions as the effector protein as part of a larger complex (the Cascade complex comprising Cse1, Cse2), that is capable of genome editing. See generally He, L., et al. (2020). Genes, 11(2), 208. Unlike other CRISPR/Cas systems, Type I systems localize to the DNA target without the Cas3 nuclease via the Cascade complex, which then recruits Cas3 to cleave DNA upon binding and locating the 3′ PAM. The Cascade complex is also responsible for processing crRNAs such that they can be used to guide it to the target position. Because of this functionality, Cascade has the ability to process multiple arrayed crRNAs from a single molecule. See. Luo, M. (2015). Nucleic Acids Research, 43(1), 674-681. As such, Type I system may be used to edit multiple targeted genes or loci from a single molecule.

Because the natural Cas3 substrate is ssDNA, its function in genomic editing is thought to be as a nickase; however, when targeted in tandem, the resulting edit is a result of blunt end cuts to opposing strands to approximate a blunt-cutting endonuclease, such as Cas9. See Pickar-Oliver, A., & Gersbach, C. A. (2019). Nature Reviews Molecular Cell Biology, 20(8), 490-507.

Like Type I nucleases, the Type III system relies upon an complex of proteins to effect nucleic acid cleavage. Particularly, Cas10 possesses the nuclease activity to cleave ssDNA in prokaryotes. See Tamulaitis, G. Trends in Microbiology, 25(1), 49-61. Interestingly, this CRISPR/Cas system, native to archaea, exhibits dual specificity and targets both ssDNA and ssRNA. Aside from this change, the system functions much like Type I in that the crRNA targets an effector complex (similar to Cascade) in a sequence-dependent manner. Similarly, the effector complex processes crRNAs prior to association. The dual nature of this nuclease makes its applications to genomic editing potentially more powerful, as both genomic DNA and, in some cases, mRNAs with the same sequence may be targeted to silence particular targeted genes.

Class 2 CRISPR/Cas Systems:

The exact components, compositions, and methods for effectuating a change in a targeted nucleic acid sequence using a Class 2 CRISPR/Cas system will vary, but should minimally include: a nuclease (selected from at least Types II, and V), at least one guide RNA selected from 1) sgRNA or 2) a combination of crRNA and tracrRNA. These CRISPR/Cas systems have been categorized together as Class 2 CRISPR/Cas systems due to their similarities in requirements and mode of action within a eukaryotic cell. To this end, compositions, components, and methods among Class 2 constituents may be considered functionally interchangeable, and the following details, provided merely for exemplary purposes, do not represent an exhaustive list of class members:

Type II nucleases are the best-characterized CRISPR/Cas systems, particularly the canonical genomic editing nuclease Cas9 (see Table 2). Multiple Cas9 proteins, derived from various bacterial species, have been isolated. The primary distinction between these nucleases is the PAM, a required recognition site within the targeted dsDNA. After association with a gRNA molecule, the crRNA (or targeting domain of a sgRNA) orients the nuclease at the proper position, but the protein's recognition of the PAM is what induces a cleavage event near that site, resulting in a blunt DSB.

In addition to the naturally-derived Cas9 proteins, several engineered variants have similarly been reported. These range from Cas9 with enhanced specific (i.e., less off-target activity), such as espCas9. Others have been catalytically modified via point mutations in the RuvC (e.g., D10A) and HNH (e.g., H840A) domains such that they induce only single-strand breaks (i.e., Cas9 nickases). See Frock, R. et al. (2015). Nature Biotechnology, 33(2), 179-186. These variants, collectively referred to herein as enhanced specificity Cas9 variants (spCas9), have also been shown to be less error-prone in editing. Such mitigation of off-target effects becomes paramount when selecting for a desired insertion (i.e., a knock in mutation, in which a desired nucleotide sequence is introduced into a target nucleic acid molecule) rather than a deletion. Indeed, less off-target effects may aid in the preferred DNA repair mechanism (HDR, in most instances for knock in mutations). See generally Naeem, M., et al. (2020). Cells, 9(7), 1608.

Additional exemplary further engineered variants of canonical Cas proteins (e.g., mutants, chimeras, and include the following (which are hereby incorporated by reference): WO2015035162A2, WO2019126716A1, WO2019126774A1, WO2014093694A1, and WO2014150624A1.

For the avoidance of doubt, spCas9 collectively refers to any one of the group consisting of espCas9 (also referred to herein as ESCas9 or esCas9), HFCas9, PECas9, arCas9.

Like the canonical Cas9 systems, Type V nucleases only require a synthetic sgRNA with a targeting domain complementary to a genomic sequence to carry out genomic editing. These nucleases contain a RuvC domain but lack the HNH domain of Type II nucleases. Further, Cas12, for example, leaves a staggered cut in the dsDNA substrate distal to the PAM, as compared to Cas9's blunt cut next to the PAM. Both Cas12a, also known as Cpf1, and Cas12b, also known as C2c1 (see Table 2), act as part of larger complex of two gRNA-associated nucleases that) acts on dsDNA as quaternary structure nicking each strand simultaneously. See Zetsche B, et al. Cell. 2015; 163(3):759-771.; see also Liu L, Chen P, Wang M, et al. Mol Cell. 2017; 65(2):310-322. Additionally, Cas12b (C2c1) is a highly accurate nuclease with little tolerance for mismatches. See Yang H, et al. Cell. 2016; 167(7):1814-1828.e12.

TABLE 2
Exemplary list of Cas nucleases and
their requirements

| | | Type of end | |
|---|---|---|---|
| | | generated | Spacer |
| | PAM | (nucleic | length |
| Nuclease (Species) | (5′→3′) | acid target) | (nt) |
| Cas9 (S. pyogenes) | NGG | Blunt (dsDNA) | 20 |
| Cas9 (S. aureus) | NNGRRT | Blunt (dsDNA) | 20 |
| Cas9 (C. jejuni) | NNNNRYAC | Blunt (dsDNA) | 22 |
| Cas9 (S. thermophilus) | NNAGAAW | Blunt (dsDNA) | 20 |
| Cas9 (N. meningitidis) | NNNNGATT | Blunt (dsDNA) | 24 |
| Cas9 (F. novicida) | NGG | Blunt (dsDNA) | 21 |
| Cas12a (L. bacterium) | TTTV | 5′ staggered | 23-25 |
| | | (dsDNA/ssDNA) | |
| Cas12a | TTTN | 5′ staggered | 24 |
| (Acidaminococcus sp.) | | (dsDNA/ssDNA) | |
| Cas3 (E. coli) | CTT/CCT/ | None/blunt | 32 |
| | CAT/CTC | (ssDNA) | |

See generally Wang, J., Zhang, C., & Feng, B. (2020). Journal of Cellular and Molecular Medicine, 24(6), 3256-3270, where N=any nucleotide; R=any purine (A or G); Y=any pyrimidine (C or T); W=A or T; V=A, C or G.

In one aspect, the CRISPR/Cas system of the present disclosure comprises at least one Cas protein derived from one or more of the following selected bacterial genera: Corynebacterium, Sutterella, Legionella, Treponema, Filifactor, Eubacterium, Streptococcus, Lactobacillus, Mycoplasma, Bacteroides, Flavobacterium, Spirochaeta, Azospirillum, Gluconacetobacter, Neisseria, Roseburia, Parvibaculum, Nitratifractor, Campylobacter, Pseudomonas, Streptomyces, Staphylococcus, Francisella, Acidaminococcus, Lachnospiraceae, Leptotrichia, and Prevotella. In some embodiments, the Cas protein is derived from Deltaproteobacteria or Planctomycetes bacterial species.

Some aspects of the present disclosure provide strategies, methods, compositions, and treatment modalities for altering a targeted sequence within a gene locus (e.g., altering the sequence of a wild type and/or of a mutant in a cell or in a patient having or experiencing effects of rheumatoid arthritis, osteoarthritis, or other inflammatory diseases of the joint or cells therein) by insertion or deletion of one or more nucleotides mediated by an RNA-guided nuclease and one or more guide RNAs (gRNAs), resulting in loss of function of the targeted gene product. Such an alteration is alternatively referred to as “knocking out” the gene of interest (i.e., generation of a “knock out”).

In certain embodiments, any region of the IL1A gene (e.g., 5′ untranslated region [UTR], exon 1, exon 2, exon 3, exon 4, exon 5, exon 6, exon 7, intronic regions, intron/exon junctions, the 3′ UTR, or polyadenylation signal) is targeted by an RNA-guided nuclease to alter the gene. In certain embodiments, any region of the IL1B gene (e.g., 5′ untranslated region [UTR], exon 1, exon 2, exon 3, exon 4, exon 5, exon 6, exon 7, intronic regions, intron/exon junctions, the 3′ UTR, or polyadenylation signal) is targeted by an RNA-guided nuclease to alter the gene. In certain embodiments, the targeted gene is selected from inflammatory effectors (e.g., IL1A, IL1B, IL1R1, IL1R2, IL6, IL18, TNF, TGFB1. In some embodiments, any region of the targeted gene (e.g., a promoter region, a 5′ untranslated region, a 3′ untranslated region, an exon, an intron, or an exon/intron border) is targeted by an RNA-guided nuclease to alter the gene. In some embodiments, a non-coding region of the targeted gene (e.g., an enhancer region, a promoter region, an intron, 5′ UTR, 3′ UTR, polyadenylation signal) is targeted to alter the gene.

CRISPR Guide RNAs:

In one aspect, the CRISPR/Cas system of the present disclosure further provides a gRNA molecule (e.g., an isolated or non-naturally occurring RNA molecule) that interacts with the Cas protein. In certain embodiments, the gRNA is an sgRNA, in which the targeting (i.e. complementary) domain, comprising a nucleotide sequence which is complementary with a target domain from a targeted gene, is incorporated into a single RNA molecule with the protein-interacting domain. In certain embodiments, the targeting domain is a crRNA that is provided to a eukaryotic cells with tracrRNA, which acts as a scaffold through interactions with both the crRNA and the nuclease. In some embodiments, the system is further, optionally, comprised of an oligonucleotide—an HDR template with homology to either side of the target position. See Bloh, K., & Rivera-Torres, N, at 3836.

In an embodiment, the targeting domain of the gRNA molecule is configured to orient an associated nuclease such that a cleavage event, (e.g., a double strand break or a single strand break) occurs sufficiently close to a target position, in the targeted gene or locus, thereby facilitating an alteration in the nucleic acid sequence. In some embodiments, the targeting domain is 20 nucleotides in length. In some embodiments, the targeting domain is 14, 15, 16, 17, 18, 19, 20, 21, 22, 23, 24, 25, 26, 27, 28, 29, or 30 nucleotides in length.

In some embodiments, the targeting domain orients the nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of a target position. The double-strand or single-strand break, may be positioned upstream or downstream of a target position, and either within or upstream a functional domain cluster within the targeted gene.

In certain embodiments, a second gRNA molecule, comprising a second targeting domain orients a second associated nuclease such that a cleavage event occurs sufficiently close to a target position, in the targeted gene or locus, thereby facilitating an alteration in the nucleic acid sequence. In an embodiment, the second gRNA molecule targets the same targeted gene or locus as the first gRNA molecule. In other embodiments, the second gRNA molecule targets a different targeted gene or locus as the first gRNA molecule. In some embodiments, the second targeting domain is 20 nucleotides in length. In some embodiments, the second targeting domain is 14, 15, 16, 17, 18, 19, 20, 21, 22, 23, 24, 25, 26, 27, 28, 29, or 30 nucleotides in length.

In some embodiments, the second targeting domain orients the nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of a target position. The double-strand or single-strand break, may be positioned upstream or downstream of a target position, and either within or upstream a functional domain cluster within the targeted gene.

In an embodiment, the targeting domains of the first and second gRNA molecules are configured such that a cleavage event is positioned, independently for each of the gRNA molecules, within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of the respective target position. In an embodiment, the first and second gRNA molecules alter the targeted nucleic acid sequences simultaneously. In an embodiment, the first and second gRNA molecules alter the targeted nucleic acid sequences sequentially.

In an embodiment, a single-strand break is accompanied by a second single-strand break, positioned by the targeting domains of a first and second gRNA molecule, respectively. For example, the targeting domains may orient the associated nucleases such that a cleavage event, (e.g., the two single-strand breaks), are positioned within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of a target position. In an embodiment, the targeting domain of a first and second gRNA molecules are configured to orient associated nucleases such that, for example, two single-strand breaks occurs at the same target position, or within 1, 2, 3, 4, 5, 10, 15, 20, 25, 30, 35, 40, 45, 50 nucleotides of one another, on opposing strands of genomic DNA, thereby essentially approximating a double strand break.

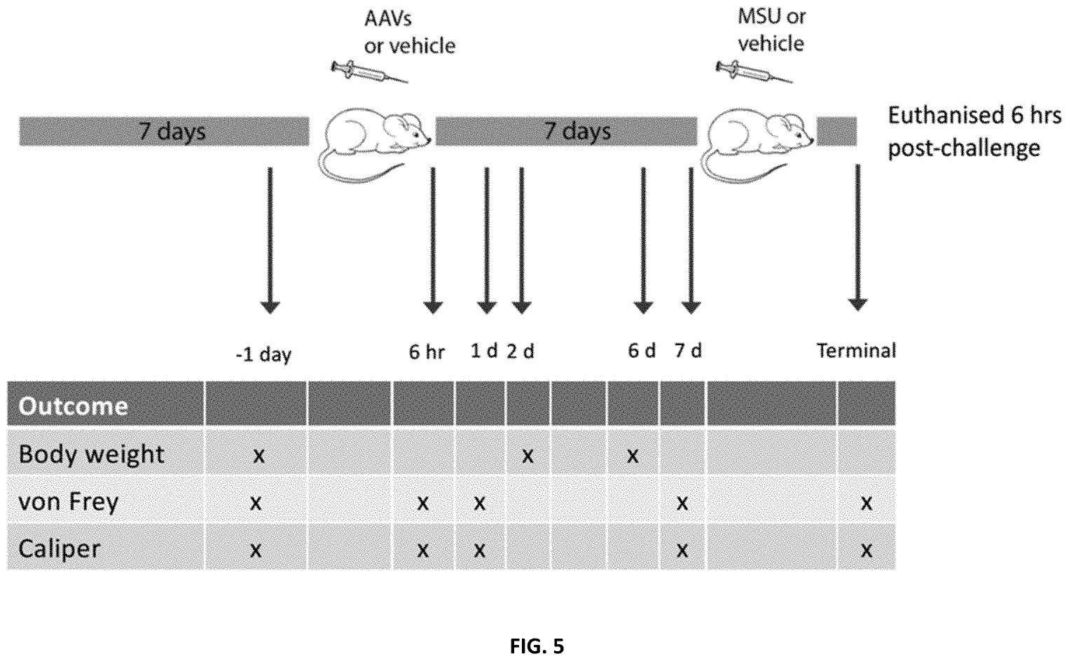

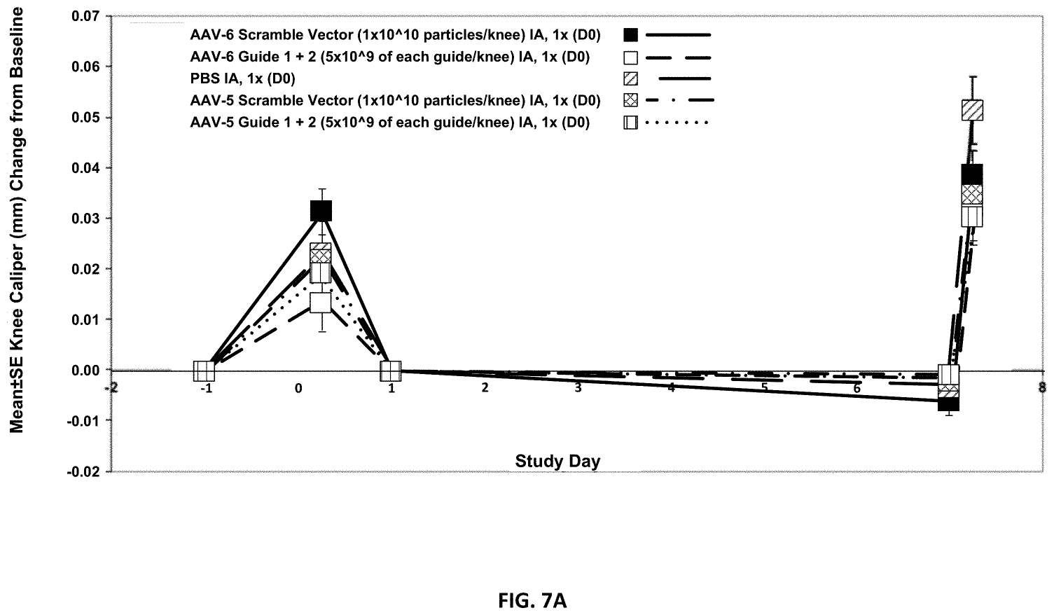

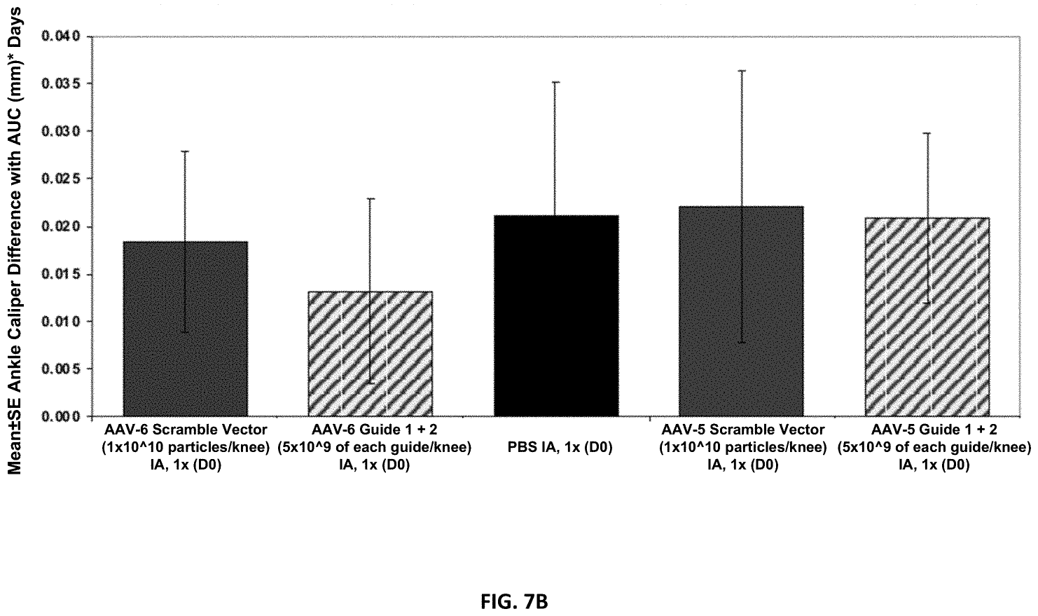

In an embodiment, a nucleic acid encodes a targeting domain of a first gRNA molecule and a targeting domain of a second gRNA molecule selected from sequences in FIG. 5. In an embodiment, a nucleic acid encodes a targeting domain of a first gRNA molecule and a targeting domain of a second gRNA molecule selected from sequences in FIG. 7. In an embodiment, a nucleic acid encodes a first sgRNA molecule. In an embodiment a nucleic acid encodes a second sgRNA molecule. In an embodiment, a nucleic acid encodes a third sgRNA molecule. In an embodiment, a nucleic acid encodes a fourth sgRNA molecule.

In an embodiment, a nucleic acid encodes a crRNA sequence of a first gRNA molecule and a crRNA sequence of a second gRNA molecule selected from sequences in FIG. 5. In an embodiment, a nucleic acid encodes a crRNA sequence of a first gRNA molecule and a crRNA sequence of a second gRNA molecule selected from sequences in FIG. 7. In an embodiment, a nucleic acid encodes a first gRNA molecule comprised of a crRNA sequence and a tracrRNA. In an embodiment, a nucleic acid encodes a second gRNA molecule comprised of a crRNA sequence and a tracrRNA. In an embodiment, a nucleic acid encodes a third gRNA molecule comprised of a crRNA sequence and a tracrRNA. In an embodiment, a nucleic acid encodes a fourth gRNA molecule comprised of a crRNA sequence and a tracrRNA.

In certain embodiments, a nucleic acid encodes a first gRNA molecule and a second gRNA molecule, comprising a chimeric gRNA molecule. In other embodiments, a nucleic acid encodes a first gRNA molecule, a second gRNA molecule, and a third gRNA molecule, comprising a chimeric gRNA molecule. In some embodiments, a nucleic acid encodes a first gRNA molecule, a second gRNA molecule, a third gRNA molecule, and a fourth gRNA molecule, comprising a chimeric gRNA molecule.

In certain embodiments, a nucleic acid may comprise (a) a sequence encoding a first gRNA molecule, comprising a targeting domain that is complementary with a target position in the targeted gene or locus, (b) a sequence encoding a second gRNA molecule, comprising a targeting domain that is complementary with a target position in the second targeted gene or locus, and (c) a sequence encoding an RNA-guided nuclease (e.g., Cas9 or other Cas protein). Optionally, (d) and (e) are sequences encoding a third and fourth gRNA molecule, respectively. In some embodiments, the second targeted gene or locus is the same as the first targeted gene or locus. In other embodiments, the second targeted gene or locus is different from the first targeted gene or locus. In some embodiments, (a), (b), and (c) are encoded within the same nucleic acid molecule (i.e., the same vector, the same viral vector, the same adeno-associated virus (AAV) vector). In some embodiments, (a) and (b) are encoded within the same nucleic acid molecule. In some embodiments, (a), (b) and (d) are encoded within the same nucleic acid molecule. In some embodiments, (a), (b) and (e) are encoded within the same nucleic acid molecule. In some embodiments, (a), (b), (d) and (e) are encoded within the same nucleic acid molecule. In some embodiments, (a), (b), and (c) are encoded within separate nucleic acid molecules. When more than two gRNAs are used, any combination of (a), (b), (c), (d) and (e) may be encoded within a single or separate nucleic acid molecules.

In an embodiment, the nucleic acid molecule is an AAV vector. Exemplary AAV vectors that may be used with any CRISPR/Cas system of the present disclosure include an AAV1 vector, an AAV2 vector, an AAV3 vector, an AAV4 vector, an AAV5 vector, an AAV6 vector, an AAV7 vector, an AAV8 vector, an AAV9 vector, an AAV10 vector.

In some embodiments, (a), (b), and (c) are encoded within the same vector. In some embodiments, (a) and (b) are encoded within the same vector. In some embodiments, (a), (b) and (d) are encoded within the same vector. In some embodiments, (a), (b) and (e) are encoded within the same vector. In some embodiments, (a), (b), (d) and (e) are encoded within the same vector. In some embodiments, (a), (b), and (c) are encoded within separate vectors. When more than two gRNAs are used, any combination of (a), (b), (c), (d) and (e) may be encoded within a single or separate vectors.

In one aspect, the nucleic acid molecules (i.e., those encoding (a), (b), (c), (d) or (e)) are delivered to a target cell (i.e., any combination of the encoded RNA-guided nuclease of (c) and at least one encoded gRNA molecule of (a), (b), (d), or (e) contact a target cell). In some embodiments, said nucleic acid molecules are delivered to a target cell in vivo. In other embodiments, said nucleic acid molecules are delivered to a target cell ex vivo. In some embodiments, said nucleic acid molecules are delivered to a target cell in vitro. In certain embodiments, said nucleic acid molecules are delivered to a target cell as DNA. In other embodiments, said nucleic acid molecules are delivered to a target cell as RNA (e.g., mRNA). In some embodiments, the products of said nucleic acid molecules are delivered as an assembled ribonucleoprotein (RNP).

In an embodiment, contacting a target cell comprises delivering said encoded RNA-guided nuclease of (c), as a protein or mRNA with at least one said nucleic acid molecules selected from (a), (b), (d), and (e).

In an embodiment, contacting a target cell comprises delivering said encoded RNA-guided nuclease of (c), as DNA with at least one said nucleic acid molecules selected from (a), (b), (d), and (e).

In certain embodiments, CRISPR components are delivered are delivered to a target cell via nanoparticles. Exemplary nanoparticles that may be used with all CRISPR/Cas systems disclosed herein include, at least, lipid nanoparticles or liposomes, hydrogel nanoparticles, metalorganic nanoparticles, gold nanoparticles, and magnetic nanoparticles. See generally Xu, C. F., et al. (2021). Advanced Drug Delivery Reviews, 168, 3-29.

Unless otherwise defined, all technical and scientific terms used herein have the same meaning as commonly understood by one of ordinary skill in the art to which this disclosure belongs. Although methods and materials similar or equivalent to those described herein may be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

The present disclosure provides compositions useful for treating joint disorders with an inflammatory component. In some aspects, the compositions are useful to prevent the progression of osteoarthritis and to treat osteoarthritis in a mammalian joint.

In some aspects, the pharmaceutical composition comprises a gene-editing system, wherein the gene-editing system causes expression the at least one genetic locus related to joint function to be silenced or reduced in at least a portion of the cells comprising the joint.

In an aspect, the pharmaceutical composition comprises a gene-editing system, wherein the gene-editing system targets one or more of IL-1α, and IL-1β. In some aspects, the pharmaceutical composition comprises a gene-editing system, wherein the gene-editing system targets one or more of TNF-α, IL-6, IL-8, IL-18, a matrix metalloproteinase (MMP), or components of the NLRP3 inflammasome.

In some aspects, the pharmaceutical composition comprises a gene-editing system, wherein the gene-editing comprises the use of a programmable nuclease that mediates the generation of a double-strand or single-strand break at the at least one locus related to joint function. In some embodiments, the gene-editing system reduces the gene expression of the targeted locus or targeted loci. In some embodiments, the at least one locus related to joint tissue is silenced or reduced in at least a portion of the cells comprising the joint.

In some aspects, the cells comprising the joint are synoviocytes. In some aspects, the cells are synovial macrophages. In some aspects, the cells are synovial fibroblasts. In some aspects at least a portion of the synoviocytes are edited. In some aspects, the cells comprising the joint are chondrocytes.

In an aspect, the pharmaceutical composition targets the one or more cytokine and/or growth factor genes is/are selected from the group comprising IL-1α, IL-1β, TNF-α, IL-6, IL-8, IL-18, a matrix metalloproteinase (MMP), or a component of the NLRP3 inflammasome. In some embodiments, the component of the NLRP3 inflammasome comprises NLRP3, ASC (apoptosis-associated speck-like protein containing a CARD), caspase-1, and combinations thereof.

Pharmaceutical compositions are also provided, wherein the gene-editing causes expression of one or more cytokine and/or growth factor genes to be enhanced in at least a portion of the cells comprising the joint, the cytokine and/or growth factor gene(s) being selected from the group comprising IL-1Ra, TIMP-1, TIMP-2, TIMP-3, TIMP-4, and combinations thereof.

In some embodiments, the pharmaceutical composition provides for gene-editing, wherein the gene-editing comprises the use of a programmable nuclease that mediates the generation of a double-strand or single-strand break at said one or more cytokine and/or growth factor genes. In some embodiments, the gene-editing comprises one or more methods selected from a CRISPR method, a TALE method, a zinc finger method, and a combination thereof.

In an aspect, the gene-editing comprises a CRISPR method. In yet other aspects, the CRISPR method is a CRISPR-Cas9 method. In some aspects, the Cas9 is mutated to enhance function.

### Animal Models of Osteoarthritis

Several animal models for osteoarthritis are known to the art. Exemplary nonlimiting animal models are summarized; however, it is understood that various models may be used. Many different species of animals are used to mimic OA, for example, studies have been conducted on mice, rats, rabbits, guinea pigs, dogs, pigs, horses, and even other animals. See, e.g., Kuyinu et al., J Orthop Surg Res. 11:19 (2016) (hereinafter “Kuyinu, 2016”).

It is understood that the various methods for inducing OA may be used in any mammal. In the mouse, spontaneous, chemically induced, surgically induced, and non-invasive induction are commonly used. E.g., Kuyinu, 2016; Bapat et al., Clin Transl Med. 7:36 (2018) (hereinafter “Bapat, 2018”); and Poulet, Curr Rheumatol Rep 18:40 (2016). In the horse, osteochondral fragment-exercise model, chemical induction, traumatic induction, and induction through overuse are commonly used. In sheep, surgical induction is most common; in the guinea pig, surgical induction, chemical induction, and spontaneous (Durkin Hartley) methods are frequently used. E.g. Bapat, 2018.

The destabilized medial meniscus (DMM) is frequently used in mice to model posttraumatic osteoarthritis, e.g. Culley et al., Methods Mol Biol. 1226:143-73 (2015). The DMM model mimics clinical meniscal injury, a known predisposing factor for the development of human OA, and permits the study of structural and biological changes over the course of the disease. Mice are an attractive model organism, because mouse strains with defined genetic backgrounds may be used. Additionally, knock-out or other genetically manipulated mouse strains may be used to evaluate the importance of various molecular pathways in the response to various OA treatment modalities and regimens. For example, STR/ort mice have features that make the strain particularly susceptible to developing OA, including, increased levels of the inflammatory cytokine IL1β, Bapat et al., Clin Transl Med. 7:36 (2018). These mice commonly develop OA in knee, ankle, elbow, and temporo-mandibular joints, Jaeger et al., Osteoarthritis Cartilage 16:607-614 (2008). Other useful mutant strains of mice are known to the skilled artisan, for example, Col9a1(−/−) mice, Allen et al., Arthritis Rheum, 60:2684-2693 (2009).

Another commonly used surgical model for OA is anterior cruciate ligament transection (ACLT) model. Little and Hunter, Nat Rev Rheumatol., 9(8):485-497 (2013). The subject's ACL is surgically transected causing joint destabilization. The anterior drawer test with the joint flexed is used to confirm that transection of the ligament has occurred. In some cases, other ligaments such as the posterior cruciate ligament, medial collateral ligament, lateral collateral ligament, and/or either meniscus may be transected. As with the DMM model, a variety of mouse strains may be used to investigate various molecular pathways.

Depending on the technical objective, animals of varying size may be selected for use. Rodents are useful because of the short time needed for skeletal maturity and consequently shorter time to develop OA following surgical or other technique to induce OA. Larger animals are particularly useful to evaluate therapeutic interventions. The anatomy in larger animals is very similar to humans; for example, in dogs the cartilage thickness is less than about half the thickness of humans; this striking similarity is exemplary of why such cartilage degeneration and osteochondral defects studies are much more useful in large animal models. E.g. McCoy, Vet. Pathol., 52:803-18 (2015); and, Pelletier et al., Therapy, 7:621-34(2010).

### Gene-Editing Processes

### Overview: Compositions to Gene-Edit Synovial Cells

Embodiments of the present disclosure are directed to methods for gene-editing synovial cells (synoviocytes), the methods comprising one or more steps of gene-editing at least a portion of the synoviocytes in a joint to treat osteoarthritis or other joint disorder. As used herein, “gene-editing,” “gene editing,” and “genome editing” refer to a type of genetic modification in which DNA is permanently modified in the genome of a cell, e.g., DNA is inserted, deleted, modified or replaced within the cell's genome. In some embodiments, gene-editing causes the expression of a DNA sequence to be silenced (sometimes referred to as a gene knockout) or inhibited/reduced (sometimes referred to as a gene knockdown). In other embodiments, gene-editing causes the expression of a DNA sequence to be enhanced (e.g., by causing over-expression). In accordance with embodiments of the present disclosure, gene-editing technology is used to reduce the expression or silence pro-inflammatory genes and/or to enhance the expression of regenerative genes.

### Interleukins

According to additional embodiments, gene-editing methods of the present disclosure may be used to increase the expression of certain interleukins, such as one or more of IL-1α, IL-1β, IL-4, IL-6, IL-8, IL-9, IL-10, IL-13, IL-18, and TNF-α. Certain interleukins have been demonstrated to augment inflammatory responses in joint tissue and are linked to disease progression.

### Expression Constructs

Expression constructs encoding one or both of guide RNAs and/or Cas9 editing enzymes can be administered in any effective carrier, e.g., any formulation or composition capable of effectively delivering the component gene to cells in vivo. Approaches include, for example, electroporation and/or insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells. In some instances, the transferred nucleic acids are stably integrated into the chromosomal DNA of the host. In other instances, particularly for adeno-associated virus vectors, stable integration into the host DNA may be a rare event, resulting into episomal expression of the transgene and transient expression of the transgene.

The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see, e.g., Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573, each of which is incorporated by reference herein in its entirety for all purposes).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors may be derived from any strain of adenovirus (e.g., Ad2, Ad3, Ad5, or Ad7 etc.), including Adenovirus serotypes from other species (e.g., mouse, dog, human, etc.) that are known to those skilled in the art. The virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Helper-dependent (HDAd) vectors can also be produced with all adenoviral sequences deleted except the origin of DNA replication at each end of the viral DNA along with packaging signal at 5-prime end of the genome downstream of the left packaging signal. HDAd vectors are constructed and propagated in the presence of a replication-competent helper adenovirus that provides the required early and late proteins necessary for replication.

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). The identification of Staphylococcus aureus (SaCas9) and other smaller Cas9 enzymes that can be packaged into adeno-associated viral (AAV) vectors that are highly stable and effective in vivo, easily produced, approved by FDA, and tested in multiple clinical trials, paves new avenues for therapeutic gene editing.

In some embodiments, nucleic acids encoding a CRISPR IL-1α or IL-1β gene editing complex (e.g., Cas9 or gRNA) are entrapped in liposomes bearing positive charges on their surface (e.g., lipofectins), which can be tagged with antibodies against cell surface antigens of the target cells. These delivery vehicles can also be used to deliver Cas9 protein/gRNA complexes.

In clinical settings, the gene delivery systems for the nucleic acids encoding a CRISPR IL-1α or IL-1β gene editing complex can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the nucleic acids encoding a CRISPR IL-1α or IL-1β gene editing complex is more limited, with introduction into the subject being quite localized. For example, the nucleic acids encoding a CRISPR IL-1α or IL-1β gene editing complex can be introduced by intra-articular injection into a joint exhibiting joint disease (e.g., osteoarthritis). In some embodiments, the nucleic acids encoding a CRISPR IL-1α or IL-1β gene editing complex are administered during or after surgery; in some embodiments, a controlled-release hydrogel comprising the nucleic acids encoding a CRISPR IL-1α or IL-1β gene editing complex is administered at the conclusion of surgery before closure to prevent reduce or eliminate osteoarthritis by providing a steady dose of the nucleic acids encoding a CRISPR IL-1α or IL-1β gene editing complex over time.

A pharmaceutical preparation of the nucleic acids encoding a CRISPR IL-1α or IL-1β gene editing complex can consist essentially of the gene delivery system (e.g., viral vector(s)) in an acceptable diluent, or can comprise a slow release matrix in which the gene delivery vehicle is embedded. Alternatively, where the complete gene delivery system can be produced intact from recombinant cells, e.g., adeno-associated viral vectors, the pharmaceutical preparation can comprise one or more cells, which produce the gene delivery system.

Preferably, the CRISPR IL-1α or IL-1β editing complex is specific, i.e., induces genomic alterations preferentially at the target site (IL-1α or IL-1β), and does not induce alterations at other sites, or only rarely induces alterations at other sites. In certain embodiments, the CRISPR IL-1α or IL-1β editing complex has an editing efficiency of at least 50%, at least 60%, at least 70%, at least 80%, at least 90%, at least 95%, or at least 99%.

The sgRNAs for use in the CRISPR/Cas system for HR typically include a guide sequence (e.g., crRNA) that is complementary to a target nucleic acid sequence (target gene locus) and a scaffold sequence (e.g., tracrRNA) that interacts with a Cas nuclease (e.g., Cas9 polypeptide) or a variant or fragment thereof. A single guide RNA (sgRNA) can include a crRNA and a tracrRNA.

Exemplary target sequences for inducing genomic alterations in the IL-1α or IL-1β gene by the CRISPR-Cas editing complex are provided in Tables 2 and 12. Exemplary guide RNAs for use with the compositions, methods, and systems of the present disclosure are provided in Tables 3 and 13.

TABLE 13
Exemplary Target IL-1α and IL-1β Gene Sequences

| Ident- | | | | Target Sequence | |
|---|---|---|---|---|---|
| ifier | Genome | Gene | Exon | 5′-3′ | PAM |
| SEQ ID | Homo | IL-1α | 4 | GCCATAGCTTACATGATAGA | AGG |
| NO: 37 | sapiens | | | | |
| SEQ ID | Homo | IL-1α | 4 | TCCTTCTATCATGTAAGCTA | TGG |
| NO: 38 | sapiens | | | | |
| SEQ ID | Homo | IL-1α | 4 | CCATGCAGCCTTCATGGAGT | GGG |
| NO: 39 | sapiens | | | | |
| SEQ ID | Homo | IL-1α | 4 | TCCATGCAGCCTTCATGGAG | TGG |
| NO: 40 | sapiens | | | | |
| SEQ ID | Homo | IL-1α | 4 | AGCTATGGCCCACTCCATGA | AGG |
| NO: 41 | sapiens | | | | |
| SEQ ID | Homo | IL-1α | 4 | ATTGATCCATGCAGCCTTCA | TGG |
| NO: 42 | sapiens | | | | |
| SEQ ID | Homo | IL-1α | 4 | CCCACTCCATGAAGGCTGCA | TGG |
| NO: 43 | sapiens | | | | |
| SEQ ID | Homo | IL-1α | 4 | GCTCTCCTTGAAGGTAAGCT | TGG |
| NO: 44 | sapiens | | | | |
| SEQ ID | Homo | IL-1α | 4 | TACCACCATGCTCTCCTTGA | AGG |
| NO: 45 | sapiens | | | | |
| SEQ ID | Homo | IL-1α | 4 | GCTTACCTTCAAGGAGAGCA | TGG |
| NO: 46 | sapiens | | | | |
| SEQ ID | Homo | IL-1α | 4 | TACCTTCAAGGAGAGCATGG | TGG |
| NO: 47 | sapiens | | | | |
| SEQ ID | Homo | IL-1α | 4 | ATGGTGGTAGTAGCAACCAA | CGG |
| NO: 48 | sapiens | | | | |
| SEQ ID | Homo | IL-1α | 4 | TGGTGGTAGTAGCAACCAAC | GGG |
| NO: 49 | sapiens | | | | |
| SEQ ID | Homo | IL-1α | 4 | CTTCTTCAGAACCTTCCCGT | TGG |
| NO: 50 | sapiens | | | | |
| SEQ ID | Homo | IL-1α | 4 | GGTAGTAGCAACCAACGGGA | AGG |
| NO: 51 | sapiens | | | | |
| SEQ ID | Homo | IL-1α | 4 | GGAAGGTTCTGAAGAAGAGA | CGG |
| NO: 52 | sapiens | | | | |
| SEQ ID | Homo | IL-1α | 4 | CTCCAGGTCATCATCAGTGA | TGG |
| NO: 53 | sapiens | | | | |
| SEQ ID | Homo | IL-1α | 4 | CATCACTGATGATGACCTGG | AGG |
| NO: 54 | sapiens | | | | |
| SEQ ID | Homo | IL-1α | 4 | AGTCATTGGCGATGGCCTCC | AGG |
| NO: 55 | sapiens | | | | |
| SEQ ID | Homo | IL-1α | 4 | TTCCTCTGAGTCATTGGCGA | TGG |
| NO: 56 | sapiens | | | | |
| SEQ ID | Homo | IL-1β | 4 | TCCCATGTGTCGAAGAAGAT | AGG |
| NO: 57 | sapiens | | | | |
| SEQ ID | Homo | IL-1β | 4 | AACCTATCTTCTTCGACACA | TGG |
| NO: 58 | sapiens | | | | |
| SEQ ID | Homo | IL-1β | 4 | ACCTATCTTCTTCGACACAT | GGG |
| NO: 59 | sapiens | | | | |
| SEQ ID | Homo | IL-1β | 4 | CTTCGACACATGGGATAACG | AGG |
| NO: 60 | sapiens | | | | |
| SEQ ID | Homo | IL-1β | 4 | GTGCAGTTCAGTGATCGTAC | AGG |
| NO: 61 | sapiens | | | | |
| SEQ ID | Homo | IL-1β | 4 | GATCACTGAACTGCACGCTC | CGG |
| NO: 62 | sapiens | | | | |
| SEQ ID | Homo | IL-1β | 4 | ATCACTGAACTGCACGCTCC | GGG |
| NO: 63 | sapiens | | | | |
| SEQ ID | Homo | IL-1β | 4 | CAAAAAAGCTTGGTGATGTC | TGG |
| NO: 64 | sapiens | | | | |
| SEQ ID | Homo | IL-1β | 4 | CCATATCCTGTCCCTGGAGG | TGG |
| NO: 65 | sapiens | | | | |
| SEQ ID | Homo | IL-1β | 4 | CTGAAAGCTCTCCACCTCCA | GGG |
| NO: 66 | sapiens | | | | |
| SEQ ID | Homo | IL-1β | 4 | GCTCCATATCCTGTCCCTGG | AGG |
| NO: 67 | sapiens | | | | |
| SEQ ID | Homo | IL-1β | 4 | AGCTCTCCACCTCCAGGGAC | AGG |
| NO: 68 | sapiens | | | | |
| SEQ ID | Homo | IL-1β | 4 | GTTGCTCCATATCCTGTCCC | TGG |
| NO: 69 | sapiens | | | | |
| SEQ ID | Homo | IL-1β | 4 | GGACAGGATATGGAGCAACA | AGG |
| NO: 70 | sapiens | | | | |
| SEQ ID | Canis | IL-1α | 3 | GTCACAGCTCATATCATAGA | AGG |
| NO: 71 | familiaris | | | | |
| SEQ ID | Canis | IL-1α | 3 | ACATGCAGTCCTCATGAAGT | GGG |
| NO: 72 | familiaris | | | | |
| SEQ ID | Canis | IL-1α | 3 | GACATGCAGTCCTCATGAAG | TGG |
| NO: 73 | familiaris | | | | |
| SEQ ID | Canis | IL-1α | 3 | GAGCTGTGACCCACTTCATG | AGG |
| NO: 74 | familiaris | | | | |
| SEQ ID | Canis | IL-1α | 3 | GGATGTCTTTGAGATTTCAG | AGG |
| NO: 75 | familiaris | | | | |
| SEQ ID | Canis | IL-1α | 3 | ATTTTCCTTGAAGGTAAGCT | GGG |
| NO: 76 | familiaris | | | | |
| SEQ ID | Canis | IL-1α | 3 | GACATCCCAGCTTACCTTCA | AGG |
| NO: 77 | familiaris | | | | |
| SEQ ID | Canis | IL-1α | 3 | CTTCAAGGAAAATGTGGTAG | TGG |
| NO: 78 | familiaris | | | | |
| SEQ ID | Canis | IL-1α | 3 | GTGGTAGTGGTGGCAGCCAA | TGG |
| NO: 79 | familiaris | | | | |
| SEQ ID | Canis | IL-1α | 3 | TGGTAGTGGTGGCAGCCAAT | GGG |
| NO: 80 | familiaris | | | | |
| SEQ ID | Canis | IL-1α | 3 | CTTCTTTAGAATCTTCCCAT | TGG |
| NO: 81 | familiaris | | | | |
| SEQ ID | Canis | IL-1α | 3 | GGAAGATTCTAAAGAAGAGA | CGG |
| NO: 82 | familiaris | | | | |
| SEQ ID | Canis | IL-1α | 3 | AATGTCTTCCAGGTCATCAT | CGG |
| NO: 83 | familiaris | | | | |
| SEQ ID | Canis | IL-1α | 3 | ATTCATCACCGATGATGACC | TGG |
| NO: 84 | familiaris | | | | |
| SEQ ID | Canis | IL-1α | 3 | ATTGCCAATGACACAGAAGA | AGG |
| NO: 85 | familiaris | | | | |
| SEQ ID | Canis | IL-1β | 4 | CCTCATCTACCAGAGAACTG | TGG |
| NO: 86 | familiaris | | | | |
| SEQ ID | Canis | IL-1β | 4 | CCACAGTTCTCTGGTAGATG | AGG |
| NO: 87 | familiaris | | | | |
| SEQ ID | Canis | IL-1β | 4 | CACAGTTCTCTGGTAGATGA | GGG |
| NO: 88 | familiaris | | | | |
| SEQ ID | Canis | IL-1β | 4 | GCTGGTGGGAGACTTGCAAC | TGG |
| NO: 89 | familiaris | | | | |
| SEQ ID | Canis | IL-1β | 4 | ACTCTTGTTACAGAGCTGGT | GGG |
| NO: 90 | familiaris | | | | |
| SEQ ID | Canis | IL-1β | 4 | GACTCTTGTTACAGAGCTGG | TGG |
| NO: 91 | familiaris | | | | |
| SEQ ID | Canis | IL-1β | 4 | TCAGACTCTTGTTACAGAGC | TGG |
| NO: 92 | familiaris | | | | |
| SEQ ID | Canis | IL-1β | 4 | AGCTCTGTAACAAGAGTCTG | AGG |
| NO: 93 | familiaris | | | | |
| SEQ ID | Canis | IL-1β | 4 | CGTGTCAGTCATTGTAGCTT | TGG |
| NO: 94 | familiaris | | | | |
| SEQ ID | Canis | IL-1β | 4 | TCCTGGAGGACCTGTGGGCA | GGG |
| NO: 95 | familiaris | | | | |
| SEQ ID | Canis | IL-1β | 4 | GCTGAAGAAGCCCTGCCCAC | AGG |
| NO: 96 | familiaris | | | | |
| SEQ ID | Canis | IL-1β | 4 | CATCCTCCTGGAGGACCTGT | GGG |
| NO: 97 | familiaris | | | | |
| SEQ ID | Canis | IL-1β | 4 | TCATCCTCCTGGAGGACCTG | TGG |
| NO: 98 | familiaris | | | | |
| SEQ ID | Canis | IL-1β | 4 | GCCCTGCCCACAGGTCCTCC | AGG |
| NO: 99 | familiaris | | | | |
| SEQ ID | Canis | IL-1β | 4 | CTGCCCACAGGTCCTCCAGG | AGG |
| NO: 100 | familiaris | | | | |
| SEQ ID | Canis | IL-1β | 4 | TCTTCAGGTCATCCTCCTGG | AGG |
| NO: 101 | familiaris | | | | |
| SEQ ID | Canis | IL-1β | 4 | TGCTCTTCAGGTCATCCTCC | TGG |
| NO: 102 | familiaris | | | | |
| SEQ ID | Canis | IL-1β | 4 | TGTAGCAAAAGATGCTCTTC | AGG |
| NO: 103 | familiaris | | | | |
| SEQ ID | Canis | IL-1β | 4 | TTTTGCTACATCTTTGAAGA | AGG |
| NO: 104 | familiaris | | | | |
| SEQ ID | Equus | IL-1α | 4 | GTCATAGCTTGCATCATAGA | AGG |
| NO: 105 | caballus | | | | |
| SEQ ID | Equus | IL-1α | 4 | CCATGCAGTCCTCAGGAAGT | GGG |
| NO: 106 | caballus | | | | |
| SEQ ID | Equus | IL-1α | 4 | TCCATGCAGTCCTCAGGAAG | TGG |
| NO: 107 | caballus | | | | |
| SEQ ID | Equus | IL-1α | 4 | AAGCTATGACCCACTTCCTG | AGG |
| NO: 108 | caballus | | | | |
| SEQ ID | Equus | IL-1α | 4 | AATGTATCCATGCAGTCCTC | AGG |
| NO: 109 | caballus | | | | |
| SEQ ID | Equus | IL-1α | 4 | CCCACTTCCTGAGGACTGCA | TGG |
| NO: 110 | caballus | | | | |
| SEQ ID | Equus | IL-1α | 4 | GGATGTCTTAGAGGTTTCAG | AGG |
| NO: 111 | caballus | | | | |
| SEQ ID | Equus | IL-1α | 4 | GTTCAGCTTGGATGTCTTAG | AGG |
| NO: 112 | caballus | | | | |
| SEQ ID | Equus | IL-1α | 4 | GCTCTCCTTGAAGTTCAGCT | TGG |
| NO: 113 | caballus | | | | |
| SEQ ID | Equus | IL-1α | 4 | GACATCCAAGCTGAACTTCA | AGG |
| NO: 114 | caballus | | | | |
| SEQ ID | Equus | IL-1α | 4 | GCTGAACTTCAAGGAGAGCG | TGG |
| NO: 115 | caballus | | | | |
| SEQ ID | Equus | IL-1α | 4 | CTTCAAGGAGAGCGTGGTGC | TGG |
| NO: 116 | caballus | | | | |
| SEQ ID | Equus | IL-1α | 4 | CAAGGAGAGCGTGGTGCTGG | TGG |
| NO: 117 | caballus | | | | |
| SEQ ID | Equus | IL-1α | 4 | GTGGTGCTGGTGGCAGCCAA | CGG |
| NO: 118 | caballus | | | | |
| SEQ ID | Equus | IL-1α | 4 | TGGTGCTGGTGGCAGCCAAC | GGG |
| NO: 119 | caballus | | | | |
| SEQ ID | Equus | IL-1α | 4 | CTTCTTCAGAGTCTTCCCGT | TGG |
| NO: 120 | caballus | | | | |
| SEQ ID | Equus | IL-1α | 4 | GGAAGACTCTGAAGAAGAGA | CGG |
| NO: 121 | caballus | | | | |
| SEQ ID | Equus | IL-1α | 4 | AATGGCTTCCAGGTCATCAT | TGG |
| NO: 122 | caballus | | | | |
| SEQ ID | Equus | IL-1α | 4 | GTTCATCACCAATGATGACC | TGG |
| NO: 123 | caballus | | | | |
| SEQ ID | Equus | IL-1α | 4 | TTCTTCTGGATCATTGGCAA | TGG |
| NO: 124 | caballus | | | | |
| SEQ ID | Equus | IL-1β | 4 | GGTGGTGGGAGATTTGCAAC | TGG |
| NO: 125 | caballus | | | | |
| SEQ ID | Equus | IL-1β | 4 | AGTCTTGTTGTAGAGGTGGT | GGG |
| NO: 126 | caballus | | | | |
| SEQ ID | Equus | IL-1β | 4 | AAGTCTTGTTGTAGAGGTGG | TGG |
| NO: 127 | caballus | | | | |
| SEQ ID | Equus | IL-1β | 4 | TGAAAGTCTTGTTGTAGAGG | TGG |
| NO: 128 | caballus | | | | |
| SEQ ID | Equus | IL-1β | 4 | GTTTGAAAGTCTTGTTGTAG | AGG |
| NO: 129 | caballus | | | | |
| SEQ ID | Equus | IL-1β | 4 | ACATGCCATGTCAATCATTG | TGG |
| NO: 130 | caballus | | | | |
| SEQ ID | Equus | IL-1β | 4 | CATGTCAATCATTGTGGCTG | TGG |
| NO: 131 | caballus | | | | |
| SEQ ID | Mus | IL-1α | 4 | GCCATAGCTTGCATCATAGA | AGG |
| NO: 132 | musculus | | | | |
| SEQ ID | Mus | IL-1α | 4 | TCCTTCTATGATGCAAGCTA | TGG |
| NO: 133 | musculus | | | | |
| SEQ ID | Mus | IL-1α | 4 | GGACATCTTTGACGTTTCAG | AGG |
| NO: 134 | musculus | | | | |
| SEQ ID | Mus | IL-1α | 4 | GATGTCCAACTTCACCTTCA | AGG |
| NO: 135 | musculus | | | | |
| SEQ ID | Mus | IL-1α | 4 | TGTCACCCGGCTCTCCTTGA | AGG |
| NO: 136 | musculus | | | | |
| SEQ ID | Mus | IL-1α | 4 | CTTCACCTTCAAGGAGAGCC | GGG |
| NO: 137 | musculus | | | | |
| SEQ ID | Mus | IL-1α | 4 | ACGTTGCTGATACTGTCACC | CGG |
| NO: 138 | musculus | | | | |
| SEQ ID | Mus | IL-1α | 4 | GTATCAGCAACGTCAAGCAA | CGG |
| NO: 139 | musculus | | | | |
| SEQ ID | Mus | IL-1α | 4 | TATCAGCAACGTCAAGCAAC | GGG |
| NO: 140 | musculus | | | | |
| SEQ ID | Mus | IL-1α | 4 | GGAAGATTCTGAAGAAGAGA | CGG |
| NO: 141 | musculus | | | | |
| SEQ ID | Mus | IL-1α | 4 | CTGCAGGTCATCTTCAGTGA | AGG |
| NO: 142 | musculus | | | | |
| SEQ ID | Mus | IL-1α | 4 | ACCTTCCAGATCATGGGTTA | TGG |
| NO: 143 | musculus | | | | |
| SEQ ID | Mus | IL-1α | 4 | CTCCTTACCTTCCAGATCAT | GGG |
| NO: 144 | musculus | | | | |
| SEQ ID | Mus | IL-1α | 4 | TCCATAACCCATGATCTGGA | AGG |
| NO: 145 | musculus | | | | |
| SEQ ID | Mus | IL-1α | 4 | AACCCATGATCTGGAAGGTA | AGG |
| NO: 146 | musculus | | | | |
| SEQ ID | Mus | IL-1β | 4 | GACAGCCCAGGTCAAAGGTT | TGG |
| NO: 147 | musculus | | | | |
| SEQ ID | Mus | IL-1β | 4 | ATCAGGACAGCCCAGGTCAA | AGG |
| NO: 148 | musculus | | | | |
| SEQ ID | Mus | IL-1β | 4 | TGCTTCCAAACCTTTGACCT | GGG |
| NO: 149 | musculus | | | | |
| SEQ ID | Mus | IL-1β | 4 | TGCTCTCATCAGGACAGCCC | AGG |
| NO: 150 | musculus | | | | |
| SEQ ID | Mus | IL-1β | 4 | TGAAGCTGGATGCTCTCATC | AGG |
| NO: 151 | musculus | | | | |
| SEQ ID | Mus | IL-1β | 4 | GCTGCTGCGAGATTTGAAGC | TGG |
| NO: 152 | musculus | | | | |
| SEQ ID | Mus | IL-1β | 4 | CATCAACAAGAGCTTCAGGC | AGG |
| NO: 153 | musculus | | | | |
| SEQ ID | Mus | IL-1β | 4 | GCAGGCAGTATCACTCATTG | TGG |
| NO: 154 | musculus | | | | |
| SEQ ID | Mus | IL-1β | 4 | AGTATCACTCATTGTGGCTG | TGG |
| NO: 155 | musculus | | | | |
| SEQ ID | Mus | IL-1β | 4 | TTGTGGCTGTGGAGAAGCTG | TGG |
| NO: 156 | musculus | | | | |
| SEQ ID | Mus | IL-1β | 4 | AAGGTCCACGGGAAAGACAC | AGG |
| NO: 157 | musculus | | | | |
| SEQ ID | Mus | IL-1β | 4 | AGCTACCTGTGTCTTTCCCG | TGG |
| NO: 158 | musculus | | | | |
| SEQ ID | Mus | IL-1β | 4 | CCTCATCCTGGAAGGTCCAC | GGG |
| NO: 159 | musculus | | | | |
| SEQ ID | Mus | IL-1β | 4 | TCCTCATCCTGGAAGGTCCA | CGG |
| NO: 160 | musculus | | | | |
| SEQ ID | Mus | IL-1β | 4 | GCTCATGTCCTCATCCTGGA | AGG |
| NO: 161 | musculus | | | | |
| SEQ ID | Mus | IL-1β | 4 | CCCGTGGACCTTCCAGGATG | AGG |
| NO: 162 | musculus | | | | |
| SEQ ID | Mus | IL-1β | 4 | AGGTGCTCATGTCCTCATCC | TGG |
| NO: 163 | musculus | | | | |
| SEQ ID | Mus | IL-1β | 4 | TTCAAAGATGAAGGAAAAGA | AGG |
| NO: 164 | musculus | | | | |
| SEQ ID | Mus | IL-1β | 4 | AGTACCTTCTTCAAAGATGA | AGG |
| NO: 165 | musculus | | | | |
| SEQ ID | Mus | IL-1β | 4 | TTTTCCTTCATCTTTGAAGA | AGG |
| NO: 167 | musculus | | | | |

TABLE 14
Exemplary crRNA sequences for CRISPR sgRNAs

| Ident- | | | | Target Sequence | |
|---|---|---|---|---|---|
| ifier | Genome | Gene | Exon | 5′-3′ | PAM |
| SEQ ID | Homo | IL-1α | 4 | GCCAUAGCUUACAUGAUAGA | AGG |
| NO: 168 | sapiens | | | | |
| SEQ ID | Homo | IL-1α | 4 | UCCUUCUAUCAUGUAAGCUA | TGG |
| NO: 169 | sapiens | | | | |
| SEQ ID | Homo | IL-1α | 4 | CCAUGCAGCCUUCAUGGAGU | GGG |
| NO: 170 | sapiens | | | | |
| SEQ ID | Homo | IL-1α | 4 | UCCAUGCAGCCUUCAUGGAG | TGG |
| NO: 171 | sapiens | | | | |
| SEQ ID | Homo | IL-1α | 4 | AGCUAUGGCCCACUCCAUGA | AGG |
| NO: 172 | sapiens | | | | |
| SEQ ID | Homo | IL-1α | 4 | AUUGAUCCAUGCAGCCUUCA | TGG |
| NO: 173 | sapiens | | | | |
| SEQ ID | Homo | IL-1α | 4 | CCCACUCCAUGAAGGCUGCA | TGG |
| NO: 174 | sapiens | | | | |
| SEQ ID | Homo | IL-1α | 4 | GCUCUCCUUGAAGGUAAGCU | TGG |
| NO: 175 | sapiens | | | | |
| SEQ ID | Homo | IL-1α | 4 | UACCACCAUGCUCUCCUUGA | AGG |
| NO: 176 | sapiens | | | | |
| SEQ ID | Homo | IL-1α | 4 | GCUUACCUUCAAGGAGAGCA | TGG |
| NO: 177 | sapiens | | | | |
| SEQ ID | Homo | IL-1α | 4 | UACCUUCAAGGAGAGCAUGG | TGG |
| NO: 178 | sapiens | | | | |
| SEQ ID | Homo | IL-1α | 4 | AUGGUGGUAGUAGCAACCAA | CGG |
| NO: 179 | sapiens | | | | |
| SEQ ID | Homo | IL-1α | 4 | UGGUGGUAGUAGCAACCAAC | GGG |
| NO: 180 | sapiens | | | | |
| SEQ ID | Homo | IL-1α | 4 | CUUCUUCAGAACCUUCCCGU | TGG |
| NO: 181 | sapiens | | | | |
| SEQ ID | Homo | IL-1α | 4 | GGUAGUAGCAACCAACGGGA | AGG |
| NO: 182 | sapiens | | | | |
| SEQ ID | Homo | IL-1α | 4 | GGAAGGUUCUGAAGAAGAGA | CGG |
| NO: 183 | sapiens | | | | |
| SEQ ID | Homo | IL-1α | 4 | CUCCAGGUCAUCAUCAGUGA | TGG |
| NO: 184 | sapiens | | | | |
| SEQ ID | Homo | IL-1α | 4 | CAUCACUGAUGAUGACCUGG | AGG |
| NO: 185 | sapiens | | | | |
| SEQ ID | Homo | IL-1α | 4 | AGUCAUUGGCGAUGGCCUCC | AGG |
| NO: 186 | sapiens | | | | |
| SEQ ID | Homo | IL-1α | 4 | UUCCUCUGAGUCAUUGGCGA | TGG |
| NO: 187 | sapiens | | | | |
| SEQ ID | Homo | IL-1β | 4 | UCCCAUGUGUCGAAGAAGAU | AGG |
| NO: 188 | sapiens | | | | |
| SEQ ID | Homo | IL-1β | 4 | AACCUAUCUUCUUCGACACA | TGG |
| NO: 189 | sapiens | | | | |
| SEQ ID | Homo | IL-1β | 4 | ACCUAUCUUCUUCGACACAU | GGG |
| NO: 190 | sapiens | | | | |
| SEQ ID | Homo | IL-1β | 4 | CUUCGACACAUGGGAUAACG | AGG |
| NO: 191 | sapiens | | | | |
| SEQ ID | Homo | IL-1β | 4 | GUGCAGUUCAGUGAUCGUAC | AGG |
| NO: 192 | sapiens | | | | |
| SEQ ID | Homo | IL-1β | 4 | GAUCACUGAACUGCACGCUC | CGG |
| NO: 193 | sapiens | | | | |
| SEQ ID | Homo | IL-1β | 4 | AUCACUGAACUGCACGCUCC | GGG |
| NO: 194 | sapiens | | | | |
| SEQ ID | Homo | IL-1β | 4 | CAAAAAAGCUUGGUGAUGUC | TGG |
| NO: 195 | sapiens | | | | |
| SEQ ID | Homo | IL-1β | 4 | CCAUAUCCUGUCCCUGGAGG | TGG |
| NO: 196 | sapiens | | | | |
| SEQ ID | Homo | IL-1β | 4 | CUGAAAGCUCUCCACCUCCA | GGG |
| NO: 197 | sapiens | | | | |
| SEQ ID | Homo | IL-1β | 4 | GCUCCAUAUCCUGUCCCUGG | AGG |
| NO: 198 | sapiens | | | | |
| SEQ ID | Homo | IL-1β | 4 | AGCUCUCCACCUCCAGGGAC | AGG |
| NO: 199 | sapiens | | | | |
| SEQ ID | Homo | IL-1β | 4 | GUUGCUCCAUAUCCUGUCCC | TGG |
| NO: 200 | sapiens | | | | |
| SEQ ID | Homo | IL-1β | 4 | GGACAGGAUAUGGAGCAACA | AGG |
| NO: 201 | sapiens | | | | |
| SEQ ID | Canis | IL-1α | 3 | GUCACAGCUCAUAUCAUAGA | AGG |
| NO: 202 | familiaris | | | | |
| SEQ ID | Canis | IL-1α | 3 | ACAUGCAGUCCUCAUGAAGU | GGG |
| NO: 203 | familiaris | | | | |
| SEQ ID | Canis | IL-1α | 3 | GACAUGCAGUCCUCAUGAAG | TGG |
| NO: 204 | familiaris | | | | |
| SEQ ID | Canis | IL-1α | 3 | GAGCUGUGACCCACUUCAUG | AGG |
| NO: 205 | familiaris | | | | |
| SEQ ID | Canis | IL-1α | 3 | GGAUGUCUUUGAGAUUUCAG | AGG |
| NO: 206 | familiaris | | | | |
| SEQ ID | Canis | IL-1α | 3 | AUUUUCCUUGAAGGUAAGCU | GGG |
| NO: 207 | familiaris | | | | |
| SEQ ID | Canis | IL-1α | 3 | GACAUCCCAGCUUACCUUCA | AGG |
| NO: 208 | familiaris | | | | |
| SEQ ID | Canis | IL-1α | 3 | CUUCAAGGAAAAUGUGGUAG | TGG |
| NO: 209 | familiaris | | | | |
| SEQ ID | Canis | IL-1α | 3 | GUGGUAGUGGUGGCAGCCAA | TGG |
| NO: 210 | familiaris | | | | |
| SEQ ID | Canis | IL-1α | 3 | UGGUAGUGGUGGCAGCCAAU | GGG |
| NO: 211 | familiaris | | | | |
| SEQ ID | Canis | IL-1α | 3 | CUUCUUUAGAAUCUUCCCAU | TGG |
| NO: 212 | familiaris | | | | |
| SEQ ID | Canis | IL-1α | 3 | GGAAGAUUCUAAAGAAGAGA | CGG |
| NO: 213 | familiaris | | | | |
| SEQ ID | Canis | IL-1α | 3 | AAUGUCUUCCAGGUCAUCAU | CGG |
| NO: 214 | familiaris | | | | |
| SEQ ID | Canis | IL-1α | 3 | AUUCAUCACCGAUGAUGACC | TGG |
| NO: 215 | familiaris | | | | |
| SEQ ID | Canis | IL-1α | 3 | AUUGCCAAUGACACAGAAGA | AGG |
| NO: 216 | familiaris | | | | |
| SEQ ID | Canis | IL-1β | 4 | CCUCAUCUACCAGAGAACUG | TGG |
| NO: 217 | familiaris | | | | |
| SEQ ID | Canis | IL-1β | 4 | CCACAGUUCUCUGGUAGAUG | AGG |
| NO: 218 | familiaris | | | | |
| SEQ ID | Canis | IL-1β | 4 | CACAGUUCUCUGGUAGAUGA | GGG |
| NO: 219 | familiaris | | | | |
| SEQ ID | Canis | IL-1β | 4 | GCUGGUGGGAGACUUGCAAC | TGG |
| NO: 220 | familiaris | | | | |
| SEQ ID | Canis | IL-1β | 4 | ACUCUUGUUACAGAGCUGGU | GGG |
| NO: 221 | familiaris | | | | |
| SEQ ID | Canis | IL-1β | 4 | GACUCUUGUUACAGAGCUGG | TGG |
| NO: 222 | familiaris | | | | |
| SEQ ID | Canis | IL-1β | 4 | UCAGACUCUUGUUACAGAGC | TGG |
| NO: 223 | familiaris | | | | |
| SEQ ID | Canis | IL-1β | 4 | AGCUCUGUAACAAGAGUCUG | AGG |
| NO: 224 | familiaris | | | | |
| SEQ ID | Canis | IL-1β | 4 | CGUGUCAGUCAUUGUAGCUU | TGG |
| NO: 225 | familiaris | | | | |
| SEQ ID | Canis | IL-1β | 4 | UCCUGGAGGACCUGUGGGCA | GGG |
| NO: 226 | familiaris | | | | |
| SEQ ID | Canis | IL-1β | 4 | GCUGAAGAAGCCCUGCCCAC | AGG |
| NO: 227 | familiaris | | | | |
| SEQ ID | Canis | IL-1β | 4 | CAUCCUCCUGGAGGACCUGU | GGG |
| NO: 228 | familiaris | | | | |
| SEQ ID | Canis | IL-1β | 4 | UCAUCCUCCUGGAGGACCUG | TGG |
| NO: 229 | familiaris | | | | |
| SEQ ID | Canis | IL-1β | 4 | GCCCUGCCCACAGGUCCUCC | AGG |
| NO: 230 | familiaris | | | | |
| SEQ ID | Canis | IL-1β | 4 | CUGCCCACAGGUCCUCCAGG | AGG |
| NO: 231 | familiaris | | | | |
| SEQ ID | Canis | IL-1β | 4 | UCUUCAGGUCAUCCUCCUGG | AGG |
| NO: 232 | familiaris | | | | |
| SEQ ID | Canis | IL-1β | 4 | UGCUCUUCAGGUCAUCCUCC | TGG |
| NO: 233 | familiaris | | | | |
| SEQ ID | Canis | IL-1β | 4 | UGUAGCAAAAGAUGCUCUUC | AGG |
| NO: 234 | familiaris | | | | |
| SEQ ID | Canis | IL-1β | 4 | UUUUGCUACAUCUUUGAAGA | AGG |
| NO: 235 | familiaris | | | | |
| SEQ ID | Equus | IL-1α | 4 | GUCAUAGCUUGCAUCAUAGA | AGG |
| NO: 236 | caballus | | | | |
| SEQ ID | Equus | IL-1α | 4 | CCAUGCAGUCCUCAGGAAGU | GGG |
| NO: 237 | caballus | | | | |
| SEQ ID | Equus | IL-1α | 4 | UCCAUGCAGUCCUCAGGAAG | TGG |
| NO: 238 | caballus | | | | |
| SEQ ID | Equus | IL-1α | 4 | AAGCUAUGACCCACUUCCUG | AGG |
| NO: 239 | caballus | | | | |
| SEQ ID | Equus | IL-1α | 4 | AAUGUAUCCAUGCAGUCCUC | AGG |
| NO: 240 | caballus | | | | |
| SEQ ID | Equus | IL-1α | 4 | CCCACUUCCUGAGGACUGCA | TGG |
| NO: 241 | caballus | | | | |
| SEQ ID | Equus | IL-1α | 4 | GGAUGUCUUAGAGGUUUCAG | AGG |
| NO: 242 | caballus | | | | |
| SEQ ID | Equus | IL-1α | 4 | GUUCAGCUUGGAUGUCUUAG | AGG |
| NO: 243 | caballus | | | | |
| SEQ ID | Equus | IL-1α | 4 | GCUCUCCUUGAAGUUCAGCU | TGG |
| NO: 244 | caballus | | | | |
| SEQ ID | Equus | IL-1α | 4 | GACAUCCAAGCUGAACUUCA | AGG |
| NO: 245 | caballus | | | | |
| SEQ ID | Equus | IL-1α | 4 | GCUGAACUUCAAGGAGAGCG | TGG |
| NO: 246 | caballus | | | | |
| SEQ ID | Equus | IL-1α | 4 | CUUCAAGGAGAGCGUGGUGC | TGG |
| NO: 247 | caballus | | | | |
| SEQ ID | Equus | IL-1α | 4 | CAAGGAGAGCGUGGUGCUGG | TGG |
| NO: 248 | caballus | | | | |
| SEQ ID | Equus | IL-1α | 4 | GUGGUGCUGGUGGCAGCCAA | CGG |
| NO: 249 | caballus | | | | |
| SEQ ID | Equus | IL-1α | 4 | UGGUGCUGGUGGCAGCCAAC | GGG |
| NO: 250 | caballus | | | | |
| SEQ ID | Equus | IL-1α | 4 | CUUCUUCAGAGUCUUCCCGU | TGG |
| NO: 251 | caballus | | | | |
| SEQ ID | Equus | IL-1α | 4 | GGAAGACUCUGAAGAAGAGA | CGG |
| NO: 252 | caballus | | | | |
| SEQ ID | Equus | IL-1α | 4 | AAUGGCUUCCAGGUCAUCAU | TGG |
| NO: 253 | caballus | | | | |
| SEQ ID | Equus | IL-1α | 4 | GUUCAUCACCAAUGAUGACC | TGG |
| NO: 254 | caballus | | | | |
| SEQ ID | Equus | IL-1α | 4 | UUCUUCUGGAUCAUUGGCAA | TGG |
| NO: 255 | caballus | | | | |
| SEQ ID | Equus | IL-1β | 4 | GGUGGUGGGAGAUUUGCAAC | TGG |
| NO: 256 | caballus | | | | |
| SEQ ID | Equus | IL-1β | 4 | AGUCUUGUUGUAGAGGUGGU | GGG |
| NO: 257 | caballus | | | | |
| SEQ ID | Equus | IL-1β | 4 | AAGUCUUGUUGUAGAGGUGG | TGG |
| NO: 258 | caballus | | | | |
| SEQ ID | Equus | IL-1β | 4 | UGAAAGUCUUGUUGUAGAGG | TGG |
| NO: 259 | caballus | | | | |
| SEQ ID | Equus | IL-1β | 4 | GUUUGAAAGUCUUGUUGUAG | AGG |
| NO: 260 | caballus | | | | |
| SEQ ID | Equus | IL-1β | 4 | ACAUGCCAUGUCAAUCAUUG | TGG |
| NO: 261 | caballus | | | | |
| SEQ ID | Equus | IL-1β | 4 | CAUGUCAAUCAUUGUGGCUG | TGG |
| NO: 262 | caballus | | | | |
| SEQ ID | Mus | IL-1α | 4 | GCCAUAGCUUGCAUCAUAGA | AGG |
| NO: 263 | musculus | | | | |
| SEQ ID | Mus | IL-1α | 4 | UCCUUCUAUGAUGCAAGCUA | TGG |
| NO: 264 | musculus | | | | |
| SEQ ID | Mus | IL-1α | 4 | GGACAUCUUUGACGUUUCAG | AGG |
| NO: 265 | musculus | | | | |
| SEQ ID | Mus | IL-1α | 4 | GAUGUCCAACUUCACCUUCA | AGG |
| NO: 266 | musculus | | | | |
| SEQ ID | Mus | IL-1α | 4 | UGUCACCCGGCUCUCCUUGA | AGG |
| NO: 267 | musculus | | | | |
| SEQ ID | Mus | IL-1α | 4 | CUUCACCUUCAAGGAGAGCC | GGG |
| NO: 268 | musculus | | | | |
| SEQ ID | Mus | IL-1α | 4 | ACGUUGCUGAUACUGUCACC | CGG |
| NO: 269 | musculus | | | | |
| SEQ ID | Mus | IL-1α | 4 | GUAUCAGCAACGUCAAGCAA | CGG |
| NO: 270 | musculus | | | | |
| SEQ ID | Mus | IL-1α | 4 | UAUCAGCAACGUCAAGCAAC | GGG |
| NO: 271 | musculus | | | | |
| SEQ ID | Mus | IL-1α | 4 | GGAAGAUUCUGAAGAAGAGA | CGG |
| NO: 272 | musculus | | | | |
| SEQ ID | Mus | IL-1α | 4 | CUGCAGGUCAUCUUCAGUGA | AGG |
| NO: 273 | musculus | | | | |
| SEQ ID | Mus | IL-1α | 4 | ACCUUCCAGAUCAUGGGUUA | TGG |
| NO: 274 | musculus | | | | |
| SEQ ID | Mus | IL-1α | 4 | CUCCUUACCUUCCAGAUCAU | GGG |
| NO: 275 | musculus | | | | |
| SEQ ID | Mus | IL-1α | 4 | UCCAUAACCCAUGAUCUGGA | AGG |
| NO: 276 | musculus | | | | |
| SEQ ID | Mus | IL-1α | 4 | AACCCAUGAUCUGGAAGGUA | AGG |
| NO: 277 | musculus | | | | |
| SEQ ID | Mus | IL-1β | 4 | GACAGCCCAGGUCAAAGGUU | TGG |
| NO: 278 | musculus | | | | |
| SEQ ID | Mus | IL-1β | 4 | AUCAGGACAGCCCAGGUCAA | AGG |
| NO: 279 | musculus | | | | |
| SEQ ID | Mus | IL-1β | 4 | UGCUUCCAAACCUUUGACCU | GGG |
| NO: 280 | musculus | | | | |
| SEQ ID | Mus | IL-1β | 4 | UGCUCUCAUCAGGACAGCCC | AGG |
| NO: 281 | musculus | | | | |
| SEQ ID | Mus | IL-1β | 4 | UGAAGCUGGAUGCUCUCAUC | AGG |
| NO: 282 | musculus | | | | |
| SEQ ID | Mus | IL-1β | 4 | GCUGCUGCGAGAUUUGAAGC | TGG |
| NO: 283 | musculus | | | | |
| SEQ ID | Mus | IL-1β | 4 | CAUCAACAAGAGCUUCAGGC | AGG |
| NO: 284 | musculus | | | | |
| SEQ ID | Mus | IL-1β | 4 | GCAGGCAGUAUCACUCAUUG | TGG |
| NO: 285 | musculus | | | | |
| SEQ ID | Mus | IL-1β | 4 | AGUAUCACUCAUUGUGGCUG | TGG |
| NO: 286 | musculus | | | | |
| SEQ ID | Mus | IL-1β | 4 | UUGUGGCUGUGGAGAAGCUG | TGG |
| NO: 287 | musculus | | | | |
| SEQ ID | Mus | IL-1β | 4 | AAGGUCCACGGGAAAGACAC | AGG |
| NO: 288 | musculus | | | | |
| SEQ ID | Mus | IL-1β | 4 | AGCUACCUGUGUCUUUCCCG | TGG |
| NO: 289 | musculus | | | | |
| SEQ ID | Mus | IL-1β | 4 | CCUCAUCCUGGAAGGUCCAC | GGG |
| NO: 290 | musculus | | | | |
| SEQ ID | Mus | IL-1β | 4 | UCCUCAUCCUGGAAGGUCCA | CGG |
| NO: 291 | musculus | | | | |
| SEQ ID | Mus | IL-1β | 4 | GCUCAUGUCCUCAUCCUGGA | AGG |
| NO: 292 | musculus | | | | |
| SEQ ID | Mus | IL-1β | 4 | CCCGUGGACCUUCCAGGAUG | AGG |
| NO: 293 | musculus | | | | |
| SEQ ID | Mus | IL-1β | 4 | AGGUGCUCAUGUCCUCAUCC | TGG |
| NO: 294 | musculus | | | | |
| SEQ ID | Mus | IL-1β | 4 | UUCAAAGAUGAAGGAAAAGA | AGG |
| NO: 295 | musculus | | | | |
| SEQ ID | Mus | IL-1β | 4 | AGUACCUUCUUCAAAGAUGA | AGG |
| NO: 296 | musculus | | | | |
| SEQ ID | Mus | IL-1β | 4 | UUUUCCUUCAUCUUUGAAGA | AGG |
| NO: 297 | musculus | | | | |

In certain embodiments, the sequence of a guide RNA (e.g., a single guide RNA or sgRNA) may be modified to increase editing efficiency and/or reduce off-target effects. In certain embodiments, the sequence of a guide RNA may vary from the target sequence by about 1 base, about 2 bases, about 3 bases, about 4 bases, about 5 bases, about 5 bases, about 6 bases, about 7 bases, about 8 bases, about 9 bases, about 10 bases, about 15 bases, or greater than about 15 bases. In certain embodiments, the sequence of a guide RNA may vary from the target sequence by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, or greater than about 20%. As used herein, variation form a target sequence may refer to the degree of complementarity.

In certain embodiments, a guide RNA used with a composition, method or system of the present disclosure is identical to a sequence as shown in any one of SEQ ID NO.: 21-34 and SEQ ID NO.: 168-297. In certain embodiments, a guide RNA used with a composition, method or system of the present disclosure is at least about 95% identical to a sequence as shown in any one of SEQ ID NO.: 21-34 and SEQ ID NO.: 168-297. In certain embodiments, a guide RNA used with a composition, method or system of the present disclosure is at least about 90% identical to a sequence as shown in any one of SEQ ID NO.: 21-34 and SEQ ID NO.: 168-297. In certain embodiments, a guide RNA used with a composition, method or system of the present disclosure is at least about 85% identical to a sequence as shown in any one of SEQ ID NO.: 21-34 and SEQ ID NO.: 168-297. In certain embodiments, a guide RNA used with a composition, method or system of the present disclosure is at least about 80% identical to a sequence as shown in any one of SEQ ID NO.: 21-34 and SEQ ID NO.: 168-297. In certain embodiments, a guide RNA used with a composition, method or system of the present disclosure is at least about 75% identical to a sequence as shown in any one of SEQ ID NO.: 21-34 and SEQ ID NO.: 168-297. In certain embodiments, a guide RNA used with a composition, method or system of the present disclosure is at least about 70% identical to a sequence as shown in any one of SEQ ID NO.: 21-34 and SEQ ID NO.: 168-297. In certain embodiments, a guide RNA used with a composition, method or system of the present disclosure is at least about 65% identical to a sequence as shown in any one of SEQ ID NO.: 21-34 and SEQ ID NO.: 168-297. In certain embodiments, a guide RNA used with a composition, method or system of the present disclosure is at least about 60% identical to a sequence as shown in any one of SEQ ID NO.: 21-34 and SEQ ID NO.: 168-297. In certain embodiments, a guide RNA used with a composition, method or system of the present disclosure is at least about 55% identical to a sequence as shown in any one of SEQ ID NO.: 21-34 and SEQ ID NO.: 168-297. In certain embodiments, a guide RNA used with a composition, method or system of the present disclosure is at least about 50% identical to a sequence as shown in any one of SEQ ID NO.: 21-34 and SEQ ID NO.: 168-297. In certain embodiments, a guide RNA used with a composition, method or system of the present disclosure is at least about 45% identical to a sequence as shown in any one of SEQ ID NO.: 21-34 and SEQ ID NO.: 168-297. In certain embodiments, a guide RNA used with a composition, method or system of the present disclosure is at least about 40% identical to a sequence as shown in any one of SEQ ID NO.: 21-34 and SEQ ID NO.: 168-297. In certain embodiments, a guide RNA used with a composition, method or system of the present disclosure is at least about 35% identical to a sequence as shown in any one of SEQ ID NO.: 21-34 and SEQ ID NO.: 168-297. In certain embodiments, a guide RNA used with a composition, method or system of the present disclosure is at least about 35% identical to a sequence as shown in any one of SEQ ID NO.: 21-34 and SEQ ID NO.: 168-297.

In certain embodiments, a guide RNA used with a composition, method or system of the present has 1 base substitution in a sequence as shown in any one of SEQ ID NO.: 21-34 and SEQ ID NO.: 168-297. In certain embodiments, a guide RNA used with a composition, method or system of the present has 2 base substitutions in a sequence as shown in any one of SEQ ID NO.: 21-34 and SEQ ID NO.: 168-297. In certain embodiments, a guide RNA used with a composition, method or system of the present has 3 base substitutions in a sequence as shown in any one of SEQ ID NO.: 21-34 and SEQ ID NO.: 168-297. In certain embodiments, a guide RNA used with a composition, method or system of the present has 4 base substitutions in a sequence as shown in any one of SEQ ID NO.: 21-34 and SEQ ID NO.: 168-297. In certain embodiments, a guide RNA used with a composition, method or system of the present has 4 base substitutions in a sequence as shown in any one of SEQ ID NO.: 21-34 and SEQ ID NO.: 168-297. In certain embodiments, a guide RNA used with a composition, method or system of the present has 6 base substitutions in a sequence as shown in any one of SEQ ID NO.: 21-34 and SEQ ID NO.: 168-297. In certain embodiments, a guide RNA used with a composition, method or system of the present has 7 base substitutions in a sequence as shown in any one of SEQ ID NO.: 21-34 and SEQ ID NO.: 168-297. In certain embodiments, a guide RNA used with a composition, method or system of the present has 8 base substitutions in a sequence as shown in any one of SEQ ID NO.: 21-34 and SEQ ID NO.: 168-297. In certain embodiments, a guide RNA used with a composition, method or system of the present has 9 base substitutions in a sequence as shown in any one of SEQ ID NO.: 21-34 and SEQ ID NO.: 168-297. In certain embodiments, a guide RNA used with a composition, method or system of the present has 10 base substitutions in a sequence as shown in any one of SEQ ID NO.: 21-34 and SEQ ID NO.: 168-297. In certain embodiments, a guide RNA used with a composition, method or system of the present has 11 base substitutions in a sequence as shown in any one of SEQ ID NO.: 21-34 and SEQ ID NO.: 168-297. In certain embodiments, a guide RNA used with a composition, method or system of the present has 12 base substitutions in a sequence as shown in any one of SEQ ID NO.: 21-34 and SEQ ID NO.: 168-297. In certain embodiments, a guide RNA used with a composition, method or system of the present has 13 base substitutions in a sequence as shown in any one of SEQ ID NO.: 21-34 and SEQ ID NO.: 168-297. In certain embodiments, a guide RNA used with a composition, method or system of the present has 14 base substitutions in a sequence as shown in any one of SEQ ID NO.: 21-34 and SEQ ID NO.: 168-297. In certain embodiments, a guide RNA used with a composition, method or system of the present has 15 base substitutions in a sequence as shown in any one of SEQ ID NO.: 21-34 and SEQ ID NO.: 168-297.

In certain embodiments, a guide RNA of the present disclosure is designed to and/or capable of knocking down an expression of a target gene as shown in any one of SEQ ID NO.: 7-20 and SEQ ID NO.: 37-167. In certain embodiments, a guide RNA of the present disclosure is designed to or capable of knocking down the human IL-1α gene by binding to at least a portion of Exon 1 of the human IL-1α gene. In certain embodiments, a guide RNA of the present disclosure is designed to or capable of knocking down the human IL-1α gene by binding to at least a portion of Exon 2 of the human IL-1α gene. In certain embodiments, a guide RNA of the present disclosure is designed to or capable of knocking down the human IL-1α gene by binding to at least a portion of Exon 3 of the human IL-1α gene. In certain embodiments, a guide RNA of the present disclosure is designed to or capable of knocking down the human IL-1α gene by binding to at least a portion of Exon 4 of the human IL-1α gene. In certain embodiments, a guide RNA of the present disclosure is designed to or capable of knocking down the human IL-1α gene by binding to at least a portion of Exon 5 of the human IL-1α gene. In certain embodiments, a guide RNA of the present disclosure is designed to or capable of knocking down the human IL-1α gene by binding to at least a portion of Exon 6 of the human IL-1α gene. In certain embodiments, a guide RNA of the present disclosure is designed to or capable of knocking down the human IL-1α gene by binding to at least a portion of Exon 7 of the human IL-1α gene. In certain embodiments, a guide RNA of the present disclosure is designed to or capable of knocking down the human IL-1α gene by binding to at least a portion of Exon 8 of the human IL-1α gene.

In certain embodiments, a guide RNA of the present disclosure is designed to or capable of knocking down the human IL-1β gene by binding to at least a portion of Exon 1 of the human IL-1β gene. In certain embodiments, a guide RNA of the present disclosure is designed to or capable of knocking down the human IL-1β gene by binding to at least a portion of Exon 2 of the human IL-1β gene. In certain embodiments, a guide RNA of the present disclosure is designed to or capable of knocking down the human IL-1β gene by binding to at least a portion of Exon 3 of the human IL-1β gene. In certain embodiments, a guide RNA of the present disclosure is designed to or capable of knocking down the human IL-1β gene by binding to at least a portion of Exon 4 of the human IL-1β gene. In certain embodiments, a guide RNA of the present disclosure is designed to or capable of knocking down the human IL-1β gene by binding to at least a portion of Exon 5 of the human IL-1β gene. In certain embodiments, a guide RNA of the present disclosure is designed to or capable of knocking down the human IL-1β gene by binding to at least a portion of Exon 6 of the human IL-1β gene. In certain embodiments, a guide RNA of the present disclosure is designed to or capable of knocking down the human IL-1β gene by binding to at least a portion of Exon 7 of the human IL-1β gene.

In certain embodiments, a guide RNA of the present disclosure is designed to and/or capable of knocking down an expression of a target gene as shown in any one of SEQ ID NO.: 7-20 and SEQ ID NO.: 37-167. In certain embodiments, a guide RNA of the present disclosure is designed to or capable of knocking down the Canis familiaris IL-1α gene by binding to at least a portion of Exon 1 of the Canis familiaris IL-1α gene. In certain embodiments, a guide RNA of the present disclosure is designed to or capable of knocking down the Canis familiaris IL-1α gene by binding to at least a portion of Exon 2 of the Canis familiaris IL-1α gene. In certain embodiments, a guide RNA of the present disclosure is designed to or capable of knocking down the Canis familiaris IL-1α gene by binding to at least a portion of Exon 3 of the Canis familiaris IL-1α gene. In certain embodiments, a guide RNA of the present disclosure is designed to or capable of knocking down the Canis familiaris IL-1α gene by binding to at least a portion of Exon 4 of the Canis familiaris IL-1α gene. In certain embodiments, a guide RNA of the present disclosure is designed to or capable of knocking down the Canis familiaris IL-1α gene by binding to at least a portion of Exon 5 of the Canis familiaris IL-1α gene. In certain embodiments, a guide RNA of the present disclosure is designed to or capable of knocking down the Canis familiaris IL-1α gene by binding to at least a portion of Exon 6 of the Canis familiaris IL-1α gene. In certain embodiments, a guide RNA of the present disclosure is designed to or capable of knocking down the Canis familiaris IL-1α gene by binding to at least a portion of Exon 7 of the Canis familiaris IL-1α gene.

In certain embodiments, a guide RNA of the present disclosure is designed to or capable of knocking down the Canis familiaris IL-1β gene by binding to at least a portion of Exon 1 of the Canis familiaris IL-1β gene. In certain embodiments, a guide RNA of the present disclosure is designed to or capable of knocking down the Canis familiaris IL-1β gene by binding to at least a portion of Exon 2 of the Canis familiaris IL-1β gene. In certain embodiments, a guide RNA of the present disclosure is designed to or capable of knocking down the Canis familiaris IL-1β gene by binding to at least a portion of Exon 3 of the Canis familiaris IL-1β gene. In certain embodiments, a guide RNA of the present disclosure is designed to or capable of knocking down the Canis familiaris IL-1β gene by binding to at least a portion of Exon 4 of the Canis familiaris IL-1β gene. In certain embodiments, a guide RNA of the present disclosure is designed to or capable of knocking down the Canis familiaris IL-1β gene by binding to at least a portion of Exon 5 of the Canis familiaris IL-1β gene. In certain embodiments, a guide RNA of the present disclosure is designed to or capable of knocking down the Canis familiaris IL-1β gene by binding to at least a portion of Exon 6 of the Canis familiaris IL-1β gene. In certain embodiments, a guide RNA of the present disclosure is designed to or capable of knocking down the Canis familiaris IL-1β gene by binding to at least a portion of Exon 7 of the Canis familiaris IL-1β gene. In certain embodiments, a guide RNA of the present disclosure is designed to or capable of knocking down the Canis familiaris IL-1β gene by binding to at least a portion of Exon 8 of the Canis familiaris IL-1β gene.

In certain embodiments, a guide RNA of the present disclosure is designed to and/or capable of knocking down an expression of a target gene as shown in any one of SEQ ID NO.: 7-20 and SEQ ID NO.: 37-167. In certain embodiments, a guide RNA of the present disclosure is designed to or capable of knocking down the Equus caballus IL-1α gene by binding to at least a portion of Exon 1 of the Equus caballus IL-1α gene. In certain embodiments, a guide RNA of the present disclosure is designed to or capable of knocking down the Equus caballus IL-1α gene by binding to at least a portion of Exon 2 of the Equus caballus IL-1α gene. In certain embodiments, a guide RNA of the present disclosure is designed to or capable of knocking down the Equus caballus IL-1α gene by binding to at least a portion of Exon 3 of the Equus caballus IL-1α gene. In certain embodiments, a guide RNA of the present disclosure is designed to or capable of knocking down the Equus caballus IL-1α gene by binding to at least a portion of Exon 4 of the Equus caballus IL-1α gene. In certain embodiments, a guide RNA of the present disclosure is designed to or capable of knocking down the Equus caballus IL-1α gene by binding to at least a portion of Exon 5 of the Equus caballus IL-1α gene. In certain embodiments, a guide RNA of the present disclosure is designed to or capable of knocking down the Equus caballus IL-1α gene by binding to at least a portion of Exon 6 of the Equus caballus IL-1α gene. In certain embodiments, a guide RNA of the present disclosure is designed to or capable of knocking down the Equus caballus IL-1α gene by binding to at least a portion of Exon 7 of the Equus caballus IL-1α gene.

In certain embodiments, a guide RNA of the present disclosure is designed to or capable of knocking down the Equus caballus IL-1β gene by binding to at least a portion of Exon 1 of the Equus caballus IL-1β gene. In certain embodiments, a guide RNA of the present disclosure is designed to or capable of knocking down the Equus caballus IL-1β gene by binding to at least a portion of Exon 2 of the Equus caballus IL-1β gene. In certain embodiments, a guide RNA of the present disclosure is designed to or capable of knocking down the Equus caballus IL-1β gene by binding to at least a portion of Exon 3 of the Equus caballus IL-1β gene. In certain embodiments, a guide RNA of the present disclosure is designed to or capable of knocking down the Equus caballus IL-1β gene by binding to at least a portion of Exon 4 of the Equus caballus IL-1β gene. In certain embodiments, a guide RNA of the present disclosure is designed to or capable of knocking down the Equus caballus IL-1β gene by binding to at least a portion of Exon 5 of the Equus caballus IL-1β gene. In certain embodiments, a guide RNA of the present disclosure is designed to or capable of knocking down the Equus caballus IL-1β gene by binding to at least a portion of Exon 6 of the Equus caballus IL-1β gene. In certain embodiments, a guide RNA of the present disclosure is designed to or capable of knocking down the Equus caballus IL-1β gene by binding to at least a portion of Exon 7 of the Equus caballus IL-1β gene.

In certain embodiments, a guide RNA of the present disclosure is designed to and/or capable of knocking down an expression of a target gene as shown in any one of SEQ ID NO.: 7-20 and SEQ ID NO.: 37-167. In certain embodiments, a guide RNA of the present disclosure is designed to or capable of knocking down the Mus musculus IL-1α gene by binding to at least a portion of Exon 1 of the Mus musculus IL-1α gene. In certain embodiments, a guide RNA of the present disclosure is designed to or capable of knocking down the Mus musculus IL-1α gene by binding to at least a portion of Exon 2 of the Mus musculus IL-1α gene. In certain embodiments, a guide RNA of the present disclosure is designed to or capable of knocking down the Mus musculus IL-1α gene by binding to at least a portion of Exon 3 of the Mus musculus IL-1α gene. In certain embodiments, a guide RNA of the present disclosure is designed to or capable of knocking down the Mus musculus IL-1α gene by binding to at least a portion of Exon 4 of the Mus musculus IL-1α gene. In certain embodiments, a guide RNA of the present disclosure is designed to or capable of knocking down the Mus musculus IL-1α gene by binding to at least a portion of Exon 5 of the Mus musculus IL-1α gene. In certain embodiments, a guide RNA of the present disclosure is designed to or capable of knocking down the Mus musculus IL-1α gene by binding to at least a portion of Exon 6 of the Mus musculus IL-1α gene. In certain embodiments, a guide RNA of the present disclosure is designed to or capable of knocking down the Mus musculus IL-1α gene by binding to at least a portion of Exon 7 of the Mus musculus IL-1α gene. In certain embodiments, a guide RNA of the present disclosure is designed to or capable of knocking down the Mus musculus IL-1α gene by binding to at least a portion of Exon 8 of the Mus musculus IL-1α gene.

In certain embodiments, a guide RNA of the present disclosure is designed to or capable of knocking down the Mus musculus IL-1β gene by binding to at least a portion of Exon 1 of the Mus musculus IL-1β gene. In certain embodiments, a guide RNA of the present disclosure is designed to or capable of knocking down the Mus musculus IL-1β gene by binding to at least a portion of Exon 2 of the Mus musculus IL-1β gene. In certain embodiments, a guide RNA of the present disclosure is designed to or capable of knocking down the Mus musculus IL-1β gene by binding to at least a portion of Exon 3 of the Mus musculus IL-1β gene. In certain embodiments, a guide RNA of the present disclosure is designed to or capable of knocking down the Mus musculus IL-1β gene by binding to at least a portion of Exon 4 of the Mus musculus IL-1β gene. In certain embodiments, a guide RNA of the present disclosure is designed to or capable of knocking down the Mus musculus IL-1 gene by binding to at least a portion of Exon 5 of the Mus musculus IL-1β gene. In certain embodiments, a guide RNA of the present disclosure is designed to or capable of knocking down the Mus musculus IL-1β gene by binding to at least a portion of Exon 6 of the Mus musculus IL-1β gene. In certain embodiments, a guide RNA of the present disclosure is designed to or capable of knocking down the Mus musculus IL-1β gene by binding to at least a portion of Exon 7 of the Mus musculus IL-1β gene.

In some instances, the sgRNA is introduced into a cell (e.g., an in vitro cell such as a primary cell for ex vivo therapy, or an in vivo cell such as in a patient) with a recombinant expression vector comprising a nucleotide sequence encoding a Cas nuclease (e.g., Cas9 polypeptide) or a variant or fragment thereof. In some embodiments, the sgRNA is complexed with a Cas nuclease (e.g., a Cas9 polypeptide) or a variant or fragment thereof to form a ribonucleoprotein (RNP)-based delivery system for introduction into a cell (e.g., an in vitro cell such as a primary cell for ex vivo therapy, or an in vivo cell such as in a patient). In other instances, the sgRNA is introduced into a cell (e.g., an in vitro cell such as a primary cell for ex vivo therapy, or an in vivo cell such as in a patient) with an mRNA encoding a Cas nuclease (e.g., Cas9 polypeptide) or a variant or fragment thereof.

Any heterologous or foreign nucleic acid (e.g., target locus-specific sgRNA and/or polynucleotide encoding a Cas9 polynucleotide) can be introduced into a cell using any method known to one skilled in the art. Such methods include, but are not limited to, electroporation, nucleofection, transfection, lipofection, transduction, microinjection, electroinjection, electrofusion, nanoparticle bombardment, transformation, conjugation, and the like.

The nucleic acid sequence of the sgRNA can be any polynucleotide sequence having sufficient complementarity with a target polynucleotide sequence (e.g., target DNA sequence) to hybridize with the target sequence and direct sequence-specific binding of a CRISPR complex to the target sequence. In some embodiments, the degree of complementarity between a guide sequence of the sgRNA and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 35 nucleotides, 40 nucleotides, 45 nucleotides, 50 nucleotides, 75 nucleotides, or more nucleotides in length. In some instances, a guide sequence is about 20 nucleotides in length. In other instances, a guide sequence is about 15 nucleotides in length. In other instances, a guide sequence is about 25 nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

The nucleic acid sequence of a sgRNA can be selected using any of the web-based software described above. Considerations for selecting a DNA-targeting RNA include the PAM sequence for the Cas nuclease (e.g., Cas9 polypeptide) to be used, and strategies for minimizing off-target modifications. Tools, such as the CRISPR Design Tool, can provide sequences for preparing the sgRNA, for assessing target modification efficiency, and/or assessing cleavage at off-target sites. Another consideration for selecting the sequence of a sgRNA includes reducing the degree of secondary structure within the guide sequence. Secondary structure may be determined by any suitable polynucleotide folding algorithm. Some programs are based on calculating the minimal Gibbs free energy. Examples of suitable algorithms include mFold (Zuker and Stiegler, Nucleic Acids Res, 9 (1981), 133-148), UNAFold package (Markham et al, Methods Mol Biol, 2008, 453:3-31) and RNAfold form the ViennaRNa Package.

The sgRNA can be about 10 to about 500 nucleotides, e.g., about 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 105 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 210 nucleotides, 220 nucleotides, 230 nucleotides, 240 nucleotides, 250 nucleotides, 260 nucleotides, 270 nucleotides, 280 nucleotides, 290 nucleotides, 300 nucleotides, 310 nucleotides, 320 nucleotides, 330 nucleotides, 340 nucleotides, 350 nucleotides, 360 nucleotides, 370 nucleotides, 380 nucleotides, 390 nucleotides, 400 nucleotides, 410 nucleotides, 420 nucleotides, 430 nucleotides, 440 nucleotides, 450 nucleotides, 460 nucleotides, 470 nucleotides, 480 nucleotides, 490 nucleotides, or about 500 nucleotides. In some embodiments, the sgRNA is about 20 to about 500 nucleotides, e.g., 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 105 nucleotides 110 nucleotides, 115 nucleotides, 120 nucleotides, 125 nucleotides, 130 nucleotides, 135 nucleotides, 140 nucleotides, 145 nucleotides, 150 nucleotides, 155 nucleotides, 160 nucleotides, 165 nucleotides, 170 nucleotides, 175 nucleotides, 180 nucleotides, 185 nucleotides, 190 nucleotides, 195 nucleotides, 200 nucleotides, 205 nucleotides, 210 nucleotides, 215 nucleotides, 220 nucleotides, 225 nucleotides, 230 nucleotides, 235 nucleotides, 240 nucleotides, 245 nucleotides, 250 nucleotides, 255 nucleotides, 260 nucleotides, 265 nucleotides, 270 nucleotides, 275 nucleotides, 280 nucleotides, 285 nucleotides, 290 nucleotides, 295 nucleotides, 300 nucleotides, 305 nucleotides, 310 nucleotides, 315 nucleotides, 320 nucleotides, 325 nucleotides, 330 nucleotides, 335 nucleotides, 340 nucleotides, 345 nucleotides, 350 nucleotides, 355 nucleotides, 360 nucleotides, 365 nucleotides, 370 nucleotides, 375 nucleotides, 380 nucleotides, 385 nucleotides, 390 nucleotides, 395 nucleotides, 400 nucleotides, 405 nucleotides, 410 nucleotides, 415 nucleotides, 420 nucleotides, 425 nucleotides, 430 nucleotides, 435 nucleotides, 440 nucleotides, 445 nucleotides, 450 nucleotides, 455 nucleotides, 460 nucleotides, 465 nucleotides, 470 nucleotides, 475 nucleotides, 480 nucleotides, 485 nucleotides, 490 nucleotides, 495 nucleotides, or 500 nucleotides. In certain embodiments, the sgRNA is about 20 to about 100 nucleotides, e.g., about 20 nucleotides, e.g., 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 51 nucleotides, 52 nucleotides, 53 nucleotides, 54 nucleotides, 55 nucleotides, 56 nucleotides, 57 nucleotides, 58 nucleotides, 59 nucleotides, 60 nucleotides, 61 nucleotides, 62 nucleotides, 63 nucleotides, 64 nucleotides, 65 nucleotides, 66 nucleotides, 67 nucleotides, 68 nucleotides, 69 nucleotides, 70 nucleotides, 71 nucleotides, 72 nucleotides, 73 nucleotides, 74 nucleotides, 75 nucleotides, 76 nucleotides, 77 nucleotides, 78 nucleotides, 79 nucleotides, 80 nucleotides, 81 nucleotides, 82 nucleotides, 83 nucleotides, 84 nucleotides, 85 nucleotides, 86 nucleotides, 87 nucleotides, 88 nucleotides, 89 nucleotides, 90 nucleotides, 91 nucleotides, 92 nucleotides, 93 nucleotides, 94 nucleotides, 95 nucleotides, 96 nucleotides, 97 nucleotides, 98 nucleotides, 99 nucleotides, or about 100 nucleotides.

The scaffold sequence can be about 10 to about 500 nucleotides, e.g., about 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 105 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 210 nucleotides, 220 nucleotides, 230 nucleotides, 240 nucleotides, 250 nucleotides, 260 nucleotides, 270 nucleotides, 280 nucleotides, 290 nucleotides, 300 nucleotides, 310 nucleotides, 320 nucleotides, 330 nucleotides, 340 nucleotides, 350 nucleotides, 360 nucleotides, 370 nucleotides, 380 nucleotides, 390 nucleotides, 400 nucleotides, 410 nucleotides, 420 nucleotides, 430 nucleotides, 440 nucleotides, 450 nucleotides, 460 nucleotides, 470 nucleotides, 480 nucleotides, 490 nucleotides, or about 500 nucleotides. In some embodiments, the scaffold sequence is about 20 to about 500 nucleotides, e.g., 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 105 nucleotides 110 nucleotides, 115 nucleotides, 120 nucleotides, 125 nucleotides, 130 nucleotides, 135 nucleotides, 140 nucleotides, 145 nucleotides, 150 nucleotides, 155 nucleotides, 160 nucleotides, 165 nucleotides, 170 nucleotides, 175 nucleotides, 180 nucleotides, 185 nucleotides, 190 nucleotides, 195 nucleotides, 200 nucleotides, 205 nucleotides, 210 nucleotides, 215 nucleotides, 220 nucleotides, 225 nucleotides, 230 nucleotides, 235 nucleotides, 240 nucleotides, 245 nucleotides, 250 nucleotides, 255 nucleotides, 260 nucleotides, 265 nucleotides, 270 nucleotides, 275 nucleotides, 280 nucleotides, 285 nucleotides, 290 nucleotides, 295 nucleotides, 300 nucleotides, 305 nucleotides, 310 nucleotides, 315 nucleotides, 320 nucleotides, 325 nucleotides, 330 nucleotides, 335 nucleotides, 340 nucleotides, 345 nucleotides, 350 nucleotides, 355 nucleotides, 360 nucleotides, 365 nucleotides, 370 nucleotides, 375 nucleotides, 380 nucleotides, 385 nucleotides, 390 nucleotides, 395 nucleotides, 400 nucleotides, 405 nucleotides, 410 nucleotides, 415 nucleotides, 420 nucleotides, 425 nucleotides, 430 nucleotides, 435 nucleotides, 440 nucleotides, 445 nucleotides, 450 nucleotides, 455 nucleotides, 460 nucleotides, 465 nucleotides, 470 nucleotides, 475 nucleotides, 480 nucleotides, 485 nucleotides, 490 nucleotides, 495 nucleotides, or 500 nucleotides. In certain embodiments, the scaffold sequence is about 20 to about 100 nucleotides, e.g., about 20 nucleotides, e.g., 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 51 nucleotides, 52 nucleotides, 53 nucleotides, 54 nucleotides, 55 nucleotides, 56 nucleotides, 57 nucleotides, 58 nucleotides, 59 nucleotides, 60 nucleotides, 61 nucleotides, 62 nucleotides, 63 nucleotides, 64 nucleotides, 65 nucleotides, 66 nucleotides, 67 nucleotides, 68 nucleotides, 69 nucleotides, 70 nucleotides, 71 nucleotides, 72 nucleotides, 73 nucleotides, 74 nucleotides, 75 nucleotides, 76 nucleotides, 77 nucleotides, 78 nucleotides, 79 nucleotides, 80 nucleotides, 81 nucleotides, 82 nucleotides, 83 nucleotides, 84 nucleotides, 85 nucleotides, 86 nucleotides, 87 nucleotides, 88 nucleotides, 89 nucleotides, 90 nucleotides, 91 nucleotides, 92 nucleotides, 93 nucleotides, 94 nucleotides, 95 nucleotides, 96 nucleotides, 97 nucleotides, 98 nucleotides, 99 nucleotides, or about 100 nucleotides.

The nucleotides of the sgRNA can include a modification in the ribose (e.g., sugar) group, phosphate group, nucleobase, or any combination thereof. In some embodiments, the modification in the ribose group comprises a modification at the 2′ position of the ribose.

In some embodiments, the nucleotide includes a 2′fluoro-arabino nucleic acid, tricycle-DNA (tc-DNA), peptide nucleic acid, cyclohexene nucleic acid (CeNA), locked nucleic acid (LNA), ethylene-bridged nucleic acid (ENA), a phosphodiamidate morpholino, or a combination thereof.

Modified nucleotides or nucleotide analogues can include sugar- and/or backbone-ribonucleotides (i.e., include modifications to the phosphate-sugar backbone). For example, the phosphodiester linkages of a native or natural RNA may be to include at least one of a nitrogen or sulfur heteroatom. In some backbone-ribonucleotides the phosphoester group connecting to adjacent ribonucleotides may be replaced by a group, e.g., of phosphothioate group. In some sugar-ribonucleotides, the 2′ moiety is a group selected from H, OR, R, halo, SH, SR, H2, HR, R₂ or ON, wherein R is C₁-C6 alkyl, alkenyl or alkynyl and halo is F, Cl, Br, or I.

In some embodiments, the nucleotide contains a sugar modification. Non-limiting examples of sugar modifications include 2′-deoxy-2′-fluoro-oligoribonucleotide (2′-fluoro-2′-deoxycytidine-5′-triphosphate, 2′-fluoro-2′-deoxyuridine-5′-triphosphate), 2′-deoxy-2′-deamine oligoribonucleotide (2′-amino-2′-deoxycytidine-5′-triphosphate, 2′-amino-2′-deoxyuridine-5′-triphosphate), 2′-O-alkyl oligoribonucleotide, 2′-deoxy-2′-C-alkyl oligoribonucleotide (2′-O-methylcytidine-5′-triphosphate, 2′-methyluridine-5′-triphosphate), 2′-C-alkyl oligoribonucleotide, and isomers thereof (2′-aracytidine-5′-triphosphate, 2′-arauridine-5′-triphosphate), azidotriphosphate (2′-azido-2′-deoxycytidine-5′-triphosphate, 2′-azido-2′-deoxyuridine-5′-triphosphate), and combinations thereof.

In some embodiments, the sgRNA contains one or more 2′-fluro, 2′-amino and/or 2′-thio modifications. In some instances, the modification is a 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, 5-amino-allyl-uridine, 5-bromo-uridine, 5-iodo-uridine, 5-methyl-cytidine, ribo-thymidine, 2-aminopurine, 2′-amino-butyryl-pyrene-uridine, 5-fluoro-cytidine, and/or 5-fluoro-uridine.

There are more than 96 naturally occurring nucleoside modifications found on mammalian RNA. See, e.g., Limbach et al., Nucleic Acids Research, 22(12):2183-2196 (1994). The preparation of nucleotides and nucleotides and nucleosides are well-known in the art and described in, e.g., U.S. Pat. Nos. 4,373,071, 4,458,066, 4,500,707, 4,668,777, 4,973,679, 5,047,524, 5,132,418, 5,153,319, 5,262,530, and 5,700,642. Numerous nucleosides and nucleotides that are suitable for use as described herein are commercially available. The nucleoside can be an analogue of a naturally occurring nucleoside. In some cases, the analogue is dihydrouridine, methyladenosine, methylcytidine, methyluridine, methylpseudouridine, thiouridine, deoxycytodine, and deoxyuridine.

In some cases, the sgRNA described herein includes a nucleobase-ribonucleotide, i.e., a ribonucleotide containing at least one non-naturally occurring nucleobase instead of a naturally occurring nucleobase. Non-limiting examples of nucleobases which can be incorporated into nucleosides and nucleotides include m5C (5-methylcytidine), m5U (5-methyluridine), m6A (N6-methyladenosine), s2U (2-thiouridine), Um (2′-O-methyluridine), m1A (1-methyl adenosine), m2A (2-methyladenosine), Am (2-1-O-methyladenosine), ms2m6A (2-methylthio-N6-methyladenosine), i6A (N6-isopentenyl adenosine), ms2i6A (2-methylthio-N6isopentenyladenosine), io6A (N6-(cis-hydroxyisopentenyl) adenosine), ms2io6A (2-methylthio-N6-(cis-hydroxyisopentenyl)adenosine), g6A (N6-glycinylcarbamoyladenosine), t6A (N6-threonyl carbamoyladenosine), ms2t6A (2-methylthio-N6-threonyl carbamoyladenosine), m6t6A (N6-methyl-N6-threonylcarbamoyladenosine), hn6A (N6.-hydroxynorvalylcarbamoyl adenosine), ms2hn6A (2-methylthio-N6-hydroxynorvalyl carbamoyladenosine), Ar(p) (2′-O-ribosyladenosine(phosphate)), I (inosine), mi 1(1-methylinosine), m′lm (1,2′-O-dimethylinosine), m3C (3-methylcytidine), Cm (2T-o-methylcytidine), s2C (2-thiocytidine), ac4C (N4-acetylcytidine), f5C (5-fonnylcytidine), m5Cm (5,2-O-dimethylcytidine), ac4Cm (N4acetyl2TOmethylcytidine), k2C (lysidine), miG (1-methylguanosine), m2G (N2-methylguanosine), m7G (7-methylguanosine), Gm (2′-O-methylguanosine), m22G (N2,N2-dimethylguanosine), m2Gm (N2,2′-O-dimethylguanosine), m22Gm (N2,N2,2′-O-trimethylguanosine), Gr(p) (2′-O-ribosylguanosine(phosphate)), yW (wybutosine), o2yW (peroxywybutosine), OHyW (hydroxywybutosine), OHyW* (under hydroxywybutosine), imG (wyosine), mimG (methylguanosine), Q (queuosine), oQ (epoxyqueuosine), galQ (galtactosyl-queuosine), manQ (mannosyl-queuosine), preQo (7-cyano-7-deazaguanosine), preQi (7-aminomethyl-7-deazaguanosine), G (archaeosine), D (dihydrouridine), m5Um (5,2′-0-dimethyluridine), s4U (4-thiouridine), m5s2U (5-methyl-2-thiouridine), s2Um (2-thio-2′-O-methyluridine), acp3U (3-(3-amino-3-carboxypropyl)uridine), ho5U (5-hydroxyuridine), mo5U (5-methoxyuridine), cmo5U (uridine 5-oxyacetic acid), mcmo5U (uridine 5-oxyacetic acid methyl ester), chm5U (5-(carboxyhydroxymethyl)uridine)), mchm5U (5-(carboxyhydroxymethyl)uridine methyl ester), mcm5U (5-methoxycarbonyl methyluridine), mcm5Um (S-methoxycarbonylmethyl-2-O-methyluridine), mcm5s2U (5-methoxycarbonylmethyl-2-thiouridine), nm5s2U (5-aminomethyl-2-thiouridine), mnm5U (5-methylaminomethyluridine), mnm5s2U (5-methylaminomethyl-2-thiouridine), mnm5se2U (5-methylaminomethyl-2-selenouridine), ncm5U (5-carbamoylmethyl uridine), ncm5Um (5-carbamoylmethyl-2′-O-methyluridine), cmnm5U (5-carboxymethylaminomethyluridine), cnmm5Um (5-carboxymethylaminomethyl-2-L-Omethyluridine), cmnm5s2U (5-carboxymethylaminomethyl-2-thiouridine), m62A (N6,N6-dimethyladenosine), Tm (2′-O-methylinosine), m4C (N4-methylcytidine), m4Cm (N4,2-O-dimethylcytidine), hm5C (5-hydroxymethylcytidine), m3U (3-methyluridine), cm5U (5-carboxymethyluridine), m6Am (N6,T-O-dimethyladenosine), rn62Am (N6,N6,0-2-trimethyladenosine), m2′7G (N2,7-dimethylguanosine), m2′2′7G (N2,N2,7-trimethylguanosine), m3Um (3,2T-O-dimethyluridine), m5D (5-methyldihydrouridine), f5Cm (5-formyl-2′-O-methylcytidine), m1Gm (1,2′-O-dimethylguanosine), m′Am (1,2-O-dimethyl adenosine)irinomethyluridine), tm5s2U (S-taurinomethyl-2-thiouridine)), imG-14 (4-demethyl guanosine), imG2 (isoguanosine), or ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxy cytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, and combinations thereof.

The sgRNA can be synthesized by any method known by one of ordinary skill in the art. In some embodiments, the sgRNA is chemically synthesized. Modified sgRNAs can be synthesized using 2-O-thionocarbamate-protected nucleoside phosphoramidites. Methods are described in, e.g., Dellinger et al., J. American Chemical Society, 133, 11540-11556 (2011); Threlfall et al., Organic & Biomolecular Chemistry, 10, 746-754 (2012); and Dellinger et al, J. American Chemical Society, 125, 940-950 (2003). Modified sgRNAs are commercially available from, e.g., TriLink BioTechnologies (San Diego, Calif.).

Additional detailed description of useful sgRNAs can be found in, e.g., Hendel et al., Nat Biotechnol, 2015, 33(9): 985-989 and Dever et al., Nature, 2016, 539: 384-389, the disclosures are herein incorporated by reference in their entirety for all purposes.

A person having skill in the art will appreciate that a guide RNA as disclosed in the present disclosure may be used in combination with any Cas protein known in the art (e.g., any Cas type, from any suitable organism or bacterial species.

The Cas protein may be a type I, type II, type III, type IV, type V, or type VI Cas protein. The Cas protein may comprise one or more domains. Non-limiting examples of domains include, a guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. The guide nucleic acid recognition and/or binding domain may interact with a guide nucleic acid. The nuclease domain may comprise catalytic activity for nucleic acid cleavage. The nuclease domain may lack catalytic activity to prevent nucleic acid cleavage. The Cas protein may be a chimeric Cas protein that is fused to other proteins or polypeptides. The Cas protein may be a chimera of various Cas proteins, for example, comprising domains from different Cas proteins.

Non-limiting examples of Cas proteins include c2c1, C2c2, c2c3, Cas1, Cas1B, Cas2, Cas3, Cas4, Cas5, Cas5e (CasD), Cash, Cas6e, Cas6f, Cas7, Cas8a, Cas8a1, Cas8a2, Cas8b, Cas8c, Cas9 (Csn1 or Csx12), Cas10, Cas10d, Cas1O, Cas1Od, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and Cul966, and homologs or modified versions thereof.

The Cas protein may be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

The Cas protein may be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida. The term, “derived,” in this instance, is defined as modified from the naturally-occurring variety of bacterial species to maintain a significant portion or significant homology to the naturally-occurring variety of bacterial species. A significant portion may be at least 10 consecutive nucleotides, at least 20 consecutive nucleotides, at least 30 consecutive nucleotides, at least 40 consecutive nucleotides, at least 50 consecutive nucleotides, at least 60 consecutive nucleotides, at least 70 consecutive nucleotides, at least 80 consecutive nucleotides, at least 90 consecutive nucleotides or at least 100 consecutive nucleotides. Significant homology may be at least 50% homologous, at last 60% homologous, at least 70% homologous, at least 80% homologous, at least 90% homologous, or at least 95% homologous. The derived species may be modified while retaining an activity of the naturally-occurring variety.

### Gene Editing Methods

As discussed above, embodiments of the present disclosure provide compositions and methods to treat joint disorders, wherein a portion of the joint cells are genetically modified via gene-editing to treat a joint disorder. Embodiments of the present disclosure embrace genetic editing through nucleotide insertion (RNA or DNA), or recombinant protein insertion, into a population of synoviocytes for both promotion of the expression of one or more proteins and inhibition of the expression of one or more proteins, as well as combinations thereof. Embodiments of the present disclosure also provide methods for delivering gene-editing compositions to joint cells, and in particular delivering gene-editing compositions to synoviocytes. There are several gene-editing technologies that may be used to genetically modify joint cells, which are suitable for use in accordance with the present disclosure.

In some embodiments, a method of genetically modifying joint cells includes the step of stable incorporation of genes for production of one or more proteins. In an embodiment, a method of genetically modifying a portion of a joint's synoviocytes includes the step of retroviral transduction. In an embodiment, a method of genetically modifying a portion of a joint's synoviocytes includes the step of lentiviral transduction. Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein. In an embodiment, a method of genetically modifying a portion of a joint's synoviocytes includes the step of gamma-retroviral transduction. Gamma-retroviral transduction systems are known in the art and are described, e.g., Cepko and Pear, Cur. Prot. Mol. Biol. 1996, 9.9.1-9.9.16, the disclosure of which is incorporated by reference herein. In an embodiment, a method of genetically modifying a portion of a joint's synoviocytes includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100x, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In some aspects, viral vectors or systems are used to introduce a gene-editing system into cells comprising a joint. In some aspects, the cells are synovial fibroblasts. In some aspects, the viral vectors are an AAV vector. In some aspects, the AAV vector comprises a serotype selected from the group consisting of: AAV1, AAV1(Y705+731F+T492V), AAV2(Y444+500+730F+T491V), AAV3(Y705+731F), AAV4, AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), AAV-ShH10, and AAV-DJ/8. In some aspects, the AAV vector comprises a serotype selected from the group consisting of: AAV1, AAV5, AAV6, AAV6 (Y705F/Y731F/T492V), AAV8, AAV9, and AAV9 (Y731F).

In some aspects, the viral vector is a lentivirus. In an aspect, the lentivirus is selected from the group consisting of: human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

In an embodiment, a method of genetically modifying a portion of a joint's synoviocytes includes the step of stable incorporation of genes for production or inhibition (e.g., silencing) of one or more proteins. In an embodiment, a method of genetically modifying a portion of a joint's synoviocytes includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In an embodiment, a method of genetically modifying a portion of a joint's synoviocytes includes the step of transfection using methods described in U.S. Pat. Nos. 5,766,902; 6,025,337; 6,410,517; 6,475,994; and 7,189,705; the disclosures of each of which are incorporated by reference herein.

According to an embodiment, the gene-editing process may comprise the use of a programmable nuclease that mediates the generation of a double-strand or single-strand break at one or more immune checkpoint genes. Such programmable nucleases enable precise genome editing by introducing breaks at specific genomic loci, i.e., they rely on the recognition of a specific DNA sequence within the genome to target a nuclease domain to this location and mediate the generation of a double-strand break at the target sequence. A double-strand break in the DNA subsequently recruits endogenous repair machinery to the break site to mediate genome editing by either non-homologous end-joining (NHEJ) or homology-directed repair (HDR). Thus, the repair of the break can result in the introduction of insertion/deletion mutations that disrupt (e.g., silence, repress, or enhance) the target gene product.

Major classes of nucleases that have been developed to enable site-specific genomic editing include zinc finger nucleases (ZFNs), transcription activator-like nucleases (TALENs), and CRISPR-associated nucleases (e.g., CRISPR-Cas9). These nuclease systems can be broadly classified into two categories based on their mode of DNA recognition: ZFNs and TALENs achieve specific DNA binding via protein-DNA interactions, whereas CRISPR systems, such as Cas9, are targeted to specific DNA sequences by a short RNA guide molecule that base-pairs directly with the target DNA and by protein-DNA interactions. See, e.g., Cox et al., Nature Medicine, 2015, Vol. 21, No. 2.

Non-limiting examples of gene-editing methods that may be used in accordance with the methods of the present disclosure include CRISPR methods, TALE methods, and ZFN methods, which are described in more detail below.

### CRISPR Methods

A pharmaceutical composition for the treatment or prevention of a joint disease or condition comprising a gene-editing system, wherein said gene-editing system targets at least one locus related to joint function, wherein the gene-editing at least a portion of a joint's synoviocytes by a CRISPR method (e.g., CRISPR-Cas9, CRISPR-Cas13a, or CRISPR/Cpf1 (also known as CRISPR-Cas12a). According to particular embodiments, the use of a CRISPR method to gene-edit joint synoviocytes causes expression of one or more immune checkpoint genes to be silenced or reduced in at least a portion of the joint's synoviocytes.

CRISPR stands for “Clustered Regularly Interspaced Short Palindromic Repeats.” A method of using a CRISPR system for gene editing is also referred to herein as a CRISPR method. There are three types of CRISPR systems which incorporate RNAs and Cas proteins, and which may be used in accordance with the present disclosure: Types II, V, and VI. The Type II CRISPR (exemplified by Cas9) is one of the most well-characterized systems.

CRISPR technology was adapted from the natural defense mechanisms of bacteria and archaea (the domain of single-celled microorganisms). These organisms use CRISPR-derived RNA and various Cas proteins, including Cas9, to foil attacks by viruses and other foreign bodies by chopping up and destroying the DNA, or RNA, of a foreign invader. A CRISPR is a specialized region of DNA with two distinct characteristics: the presence of nucleotide repeats and spacers. Repeated sequences of nucleotides are distributed throughout a CRISPR region with short segments of foreign DNA (spacers) interspersed among the repeated sequences. In the type II CRISPR-Cas system, spacers are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Target recognition by the Cas9 protein requires a “seed” sequence within the crRNA and a conserved dinucleotide-containing protospacer adjacent motif (PAM) sequence upstream of the crRNA-binding region. The CRISPR-Cas system can thereby be retargeted to cleave virtually any DNA sequence by redesigning the crRNA. The crRNA and tracrRNA in the native system can be simplified into a single guide RNA (sgRNA) of approximately 100 nucleotides for use in genetic engineering. The CRISPR-Cas system is directly portable to human cells by co-delivery of plasmids expressing the Cas9 endo-nuclease and the necessary crRNA and tracrRNA (or sgRNA) components. Different variants of Cas proteins may be used to reduce targeting limitations (e.g., orthologs of Cas9, such as Cpf1).

CRISPR-Cas Mediated Homologous Recombination

The CRISPR-Cas system for homologous recombination (HR) includes a Cas nuclease (e.g., Cas9 nuclease) or a variant or fragment thereof, a DNA-targeting RNA (e.g., single guide RNA (sgRNA)) containing a guide sequence that targets the Cas nuclease to the target genomic DNA and a scaffold sequence that interacts with the Cas nuclease, and a donor template. The CRISPR-Cas system can be utilized to create a double-strand break at a desired target gene locus in the genome of a cell, and harness the cell's endogenous mechanisms to repair the induced break by homology-directed repair (HDR).

The CRISPR-Cas9 nuclease can facilitate locus-specific chromosomal integration of exogenous DNA delivered by AAV vectors. Typically, the size of the exogenous DNA (e.g., transgene, expression cassette, and the like) that can be integrated is limited by the DNA packaging capacity of an AAV vector which is about 4.0 kb. With the inclusion of two homology arms that are necessary for homologous recombination, a single AAV vector can only deliver less than about 3.7 kb of exogenous DNA. The method described herein allows for the delivery of exogenous DNA that is 4 kb or longer by splitting the nucleotide sequence between two different AAV vectors. The donor templates are designed for sequential homologous recombination events that can integrate and fuse the two parts of the nucleotide sequence.

Homologous recombination of the present disclosure can be performed using an engineered nuclease system for genome editing such as, but not limited to, CRISPR-Cas nucleases, zinc finger nucleases (ZFNs), transcription activator-like effector nucleases (TALENs), engineered mega-nucleases. In one aspect, a CRISPR-Cas-based nuclease system is used. Detailed descriptions of useful nuclease system can be found, e.g., in Gaj et al., Trends Biotechnol, 2013, July: 31(7):397-405.

Any suitable CRISPR/Cas system may be used for the methods and compositions disclosed herein. The CRISPR/Cas system may be referred to using a variety of naming systems. Exemplary naming systems are provided in Makarova, K. S. et al, “An updated evolutionary classification of CRISPR-Cas systems,” Nat Rev Microbiol (2015) 13:722-736 and Shmakov, S. et al, “Discovery and Functional Characterization of Diverse Class 2 CRISPR-Cas Systems,” Mol Cell (2015) 60:1-13. The CRISPR/Cas system may be a type I, a type II, a type III, a type IV, a type V, a type VI system, or any other suitable CRISPR/Cas system. The CRISPR/Cas system as used herein may be a Class 1, Class 2, or any other suitably classified CRISPR/Cas system. The Class 1 CRISPR/Cas system may use a complex of multiple Cas proteins to effect regulation. The Class 1 CRISPR/Cas system may comprise, for example, type I (e.g., I, IA, IB, IC, ID, IE, IF, IU), type III (e.g., III, IIA, IIIB, IIIC, IIID), and type IV (e.g., IV, IVA, IVB) CRISPR/Cas type. The Class 2 CRISPR/Cas system may use a single large Cas protein to effect regulation. The Class 2 CRISPR/Cas systems may comprise, for example, type II (e.g., II, IIA, IIB) and type V CRISPR/Cas type. CRISPR systems may be complementary to each other, and/or can lend functional units in trans to facilitate CRISPR locus targeting.

In some embodiments, a nucleotide sequence encoding the Cas nuclease is present in a recombinant expression vector. In certain instances, the recombinant expression vector is a viral construct, e.g., a recombinant adeno-associated virus construct, a recombinant adenoviral construct, a recombinant lentiviral construct, etc. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. Useful expression vectors are known to those of skill in the art, and many are commercially available. The following vectors are provided by way of example for eukaryotic host cells: pXT1, pSG5, pSVK3, pBPV, pMSG, and pSVLSV40. However, any other vector may be used if it is compatible with the host cell. For example, useful expression vectors containing a nucleotide sequence encoding a Cas9 enzyme are commercially available from, e.g., Addgene, Life Technologies, Sigma-Aldrich, and Origene.

Host cells are necessary for generating infectious AAV vectors as well as for generating AAV virions based on the disclosed AAV vectors. Various host cells are known in the art and find use in the methods of the present disclosure. Any host cells described herein or known in the art can be employed with the compositions and methods described herein.

In some embodiments, the host cell for use in generating infectious virions can be selected from any biological organism, including prokaryotic (e.g., bacterial) cells, and eukaryotic cells, including, insect cells, yeast cells and mammalian cells. A variety of cells, e.g., mammalian cells, including, e.g., murine cells, and primate cells (e.g., human cells) can be used. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, CHO, 293, Vero, NIH 3T3, PC12, Huh-7 Saos, C2C12, RAT1, Sf9, L cells, HT1080, human embryonic kidney (HEK), human embryonic stem cells, human adult tissue stem cells, pluripotent stem cells, induced pluripotent stem cells, reprogrammed stem cells, organoid stem cells, bone marrow stem cells, HLHepG2, HepG2 and primary fibroblast, hepatocyte and myoblast cells derived from mammals including human, monkey, mouse, rat, rabbit, and hamster. The requirement for the cell used is it is capable of infection or transfection by an AAV vector. In some embodiments, the host cell is one that has rep and cap stably transfected in the cell.

In some embodiments, the preparation of a host cell according to the disclosure involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus and AAV genomes, combined with polymerase chain reaction, synthetic methods, and any other suitable methods for providing the desired nucleotide sequence.

In addition to the AAV vector, the host cell can contain sequences to drive expression of the AAV capsid polypeptide (in the host cell and rep (replication) sequences of the same serotype as the serotype of the AAV Inverted Terminal Repeats (ITRs) found in the AAV vector, or a cross-complementing serotype. The AAV capsid and rep (replication) sequences may be independently obtained from an AAV source and may be introduced into the host cell in any manner known to one of skill in the art or as described herein. Additionally, when pseudotyping an AAV vector in an AAV8 capsid for example, the sequences encoding each of the essential rep (replication) proteins may be supplied by AAV8, or the sequences encoding the rep (replication) proteins may be supplied by different AAV serotypes (e.g., AAV1, AAV2, AAV3, AAV4, AAV5, AAV6, AAV7, and/or AAV9).

In some embodiments, the host cell stably contains the capsid protein under the control of a suitable promoter. In some embodiments, the capsid protein is supplied to the host cell in trans. When delivered to the host cell in trans, the capsid protein may be delivered via a plasmid containing the sequences necessary to direct expression of the selected capsid protein in the host cell. In some embodiments, when delivered to the host cell in trans, the vector encoding the capsid protein also carries other sequences required for packaging the AAV, e.g., the rep (replication) sequences.

In some embodiments, the host cell stably contains the rep (replication) sequences under the control of a suitable promoter. In another embodiment, the rep (replication) proteins are supplied to the host cell in trans. When delivered to the host cell in trans, the rep (replication) proteins may be delivered via a plasmid containing the sequences necessary to direct expression of the selected rep (replication) proteins in the host cell. In some embodiments, when delivered to the host cell in trans, the vector encoding the capsid protein (also carries other sequences required for packaging the AAV vector, e.g., the rep (replication) sequences.

In some embodiments, the rep (replication) and capsid sequences may be transfected into the host cell on a single nucleic acid molecule and exist stably in the cell as an unintegrated episome. In another embodiment, the rep (replication) and capsid sequences are stably integrated into the chromosome of the cell. Another embodiment has the rep (replication) and capsid sequences transiently expressed in the host cell. For example, a useful nucleic acid molecule for such transfection comprises, from 5′ to 3′, a promoter, an optional spacer interposed between the promoter and the start site of the rep (replication) gene sequence, an AAV rep (replication) gene sequence, and an AAV capsid gene sequence.

Although the molecule(s) providing rep (replication) and capsid can exist in the host cell transiently (i.e., through transfection), in some embodiments, one or both of the rep (replication) and capsid proteins and the promoter(s) controlling their expression be stably expressed in the host cell, e.g., as an episome or by integration into the chromosome of the host cell. The methods employed for constructing embodiments of the disclosure are conventional genetic engineering or recombinant engineering techniques such as those described in the references above.

A variety of methods of generating AAV virions are known in the art and can be used to generate AAV virions comprising the AAV vectors described herein. Generally, the methods involved inserting or transducing an AAV vector of the disclosure into a host cell capable of packaging the AAV vector into and AAV virion. Exemplary methods are described and referenced below; however, any method known to one of skill in the art can be employed to generate the AAV virions of the disclosure.

An AAV vector comprising a heterologous nucleic acid (e.g., a donor template) and used to generate an AAV virion can be constructed using methods that are well known in the art. See, e.g., Koerber et al. (2009) Mol. Ther., 17:2088; Koerber et al. (2008) Mol Ther., 16: 1703-1709; as well as U.S. Pat. Nos. 7,439,065, 6,951,758, and 6,491,907. For example, the heterologous sequence(s) can be directly inserted into an AAV genome with the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Curr. Topics Microbiol. Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

In order to produce AAV virions, an AAV vector is introduced into a suitable host cell using known techniques, such as by transfection. A number of transfection techniques are generally known in the art. See, e.g., Graham et al. (1973) Virology, 52:456, Sambrook et al. (1989) Molecular Cloning, A Laboratory Manual, Cold Spring Harbor Laboratories, New York, Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi, M. R. (1980) Cell 22:479-488), electroporation (Shigekawa et al. (1988) BioTechniques 6:742-751), liposome mediated gene transfer (Mannino et al. (1988) BioTechniques 6:682-690), lipid-mediated transduction (Felgner et al. (1987) Proc. Natl. Acad. Sci. USA 84:7413-7417), and nucleic acid delivery using high-velocity microprojectiles (Klein et al. (1987) Nature 327:70-73).

Depending on the expression system used, any of a number of transcription and translation control elements, including promoter, transcription enhancers, transcription terminators, and the like, may be used in the expression vector. Useful promoters can be derived from viruses, or any organism, e.g., prokaryotic or eukaryotic organisms. Suitable promoters include, but are not limited to, the SV40 early promoter, mouse mammary tumor virus long terminal repeat (LTR) promoter, adenovirus major late promoter (Ad MLP), a herpes simplex virus (HSV) promoter, a cytomegalovirus (CMV) promoter (such as the CMV immediate early promoter region; CMVIE), a rous sarcoma virus (RSV) promoter, a human U6 small nuclear promoter (U6), an enhanced U6 promoter, and a human HI promoter (HI), etc.

In some embodiments, polynucleotide encoding a Cas nuclease can be used in the present disclosure. Such a polynucleotide (e.g., mRNA) can be commercially obtained from, for example, TriLink BioTechnologies, GE Dharmacon, ThermoFisher, and the like.

In certain embodiments, a Cas nuclease (e.g., Cas9 polypeptide) can be used in the present disclosure. Detailed description of useful Cas9 polypeptides can be found in, e.g., Hendel et al., Nat Biotechnol, 2015, 33(9): 985-989 and Dever et al., Nature, 2016, 539: 384-389, the disclosures are herein incorporated by reference in their entirety for all purposes.

In some embodiments, a Cas nuclease (e.g., Cas9 polypeptide) is complexed with a sgRNA to form a Cas ribonucleoprotein (e.g., Cas9 ribonucleoprotein). The molar ratio of Cas nuclease to sgRNA can be any range that facilitates sequential homologous recombination of the targeting AAV vectors and target genetic locus. In some embodiments, the molar ratio of Cas9 polypeptide to sgRNA is about 1:5; 1:4; 1:3; 1:2.5; 1:2; or 1:1. In other embodiments, the molar ratio of Cas9 polypeptide to sgRNA is about 1:2 to about 1:3. In certain embodiments, the molar ratio of Cas9 polypeptide to sgRNA is about 1:2.5.

The Cas nuclease and variants or fragments thereof can be introduced into a cell (e.g., a cell isolated from a subject, or an in vivo cell such as in a subject) as a Cas polypeptide or a variant or fragment thereof, an mRNA encoding a Cas polypeptide or a variant or fragment thereof, a recombinant expression vector comprising a nucleotide sequence encoding a Cas polypeptide or a variant or fragment thereof, or a Cas ribonucleoprotein. One skilled in the art would recognize that any method of delivering an exogenous polynucleotide, polypeptide, or a ribonucleoprotein can be used. Non-limiting examples of such methods include electroporation, nucleofection, transfection, lipofection, transduction, microinjection, electroinjection, electrofusion, nanoparticle bombardment, transformation, conjugation, and the like.

In one aspect, the present disclosure provides for the use of nanoparticles as a means of delivering CRISPR components to a subject in need thereof. In some embodiments, the nanoparticles are selected from, lipid nanoparticles (LNPs) or liposomes, hydrogel nanoparticles, metalorganic nanoparticles, gold nanoparticles, and magnetic nanoparticles, See, e.g. Xu, C. F., et al. (2021). Advanced Drug Delivery Reviews, 168, 3-29; see also Buschmann et al. (2021). Vaccines 9:65; Kenjo, E., et al. (2021). Nature Communications, 12(1), 7101.

In some embodiments, CRISPR components are delivered by a nanoparticle. Without wishing to be bound by any particular theory, in certain embodiments, nucleic acids, when present in the nanoparticle, are resistant in aqueous solution to degradation with a nuclease. Lipid nanoparticles comprising nucleic acids and their method of preparation is disclosed in at least WO2017/019935, WO2017/049074, WO2017/201346, WO2017/218704, WO2018/006052, WO2018/013525, WO2018/089540, WO2018/119115, WO2018/126084, WO2018/157009, WO2018/170336, WO2018/222890, WO2019/046809, WO2019/089828, WO2020/061284, WO2020/061317, WO2020/081938, WO2020/097511, WO2020/097520, WO2020/097540, WO2020/097548, WO2020/214946, WO2020/219941, WO2020/232276, WO2020/227615, WO2020/061295, WO2021/007278, WO2021/016430, WO2021/021988, EP Patent No. EP 2 972 360, US20200155691, US20200237671, U.S. Pat. Nos. 8,058,069, 8,492,359, 8,822,668, 9,364,435, 9,404,127, 9,504,651, 9,593,077, 9,738,593, 9,868,691, 9,868,692, 9,950,068, 10,138,213, 10,166,298, 10,221,127, 10,238,754, 10,266,485, 10,383,952, 10,730,924, 10,766,852, 11,141,378 and 11,246,933, which are incorporated herein by reference in their entirety for all purposes.

### Lipid Nanoparticle Compositions

In some embodiments, the largest dimension of a nanoparticle composition is 1 micrometer or shorter (e.g., 1 micrometer, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method. Nanoparticle compositions include, for example, lipid nanoparticles (LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers may be functionalized and/or crosslinked to one another. Lipid bilayers may include one or more ligands, proteins, or channels.

In various embodiments, lipid nanoparticles described herein have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic.

In certain embodiments, the lipid nanoparticles described herein comprise one or more components, including a lipid component, and (optionally) a structural component. The lipid component comprises lipids selected from ionizable and/or cationic lipids (i.e., lipids that may have a positive or partial positive charge at physiological pH), neutral lipids (e.g., phospholipids, or sphingolipids), and polymer-conjugated lipids (e.g., PEGylated lipids). In some embodiments, the lipid component comprises a single ionizable lipid. In other embodiments, the lipid component comprises 2, 3, 4, 5, 6, 7, 8, 9, 10, 15, or 20 ionizable lipids. In some embodiments, the lipid component comprises a single neutral lipid. In other embodiments, the lipid component comprises 2, 3, 4, 5, 6, 7, 8, 9, 10, 15, or 20 neutral lipids. In some embodiments, the lipid component comprises a single polymer-conjugated lipid. In other embodiments, the lipid component comprises 2, 3, 4, 5, 6, 7, 8, 9, 10, 15, or 20 polymer-conjugated lipids. In some embodiments, the structural component comprises a single structural lipid. In other embodiments, the structural component comprises 2, 3, 4, 5, 6, 7, 8, 9, 10, 15, or 20 structural lipids. In some embodiments, the lipid component comprises at least one cationic lipid, at least one neutral lipid, and at least one polymer-conjugated lipid. The present disclosure contemplates that the lipid component may comprise any combination of the foregoing constituents.

### Ionizable/Cationic Lipids

In some embodiments, the lipid component comprises an ionizable lipid. In some embodiments, the ionizable lipid is anionic. In other embodiments, the ionizable lipid is a cationic lipid. In some embodiments, the lipid component comprises cationic lipids including, but not limited to, a cationic lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and mixtures thereof.

Non-exhaustive and non-limiting examples of cationic lipids include:

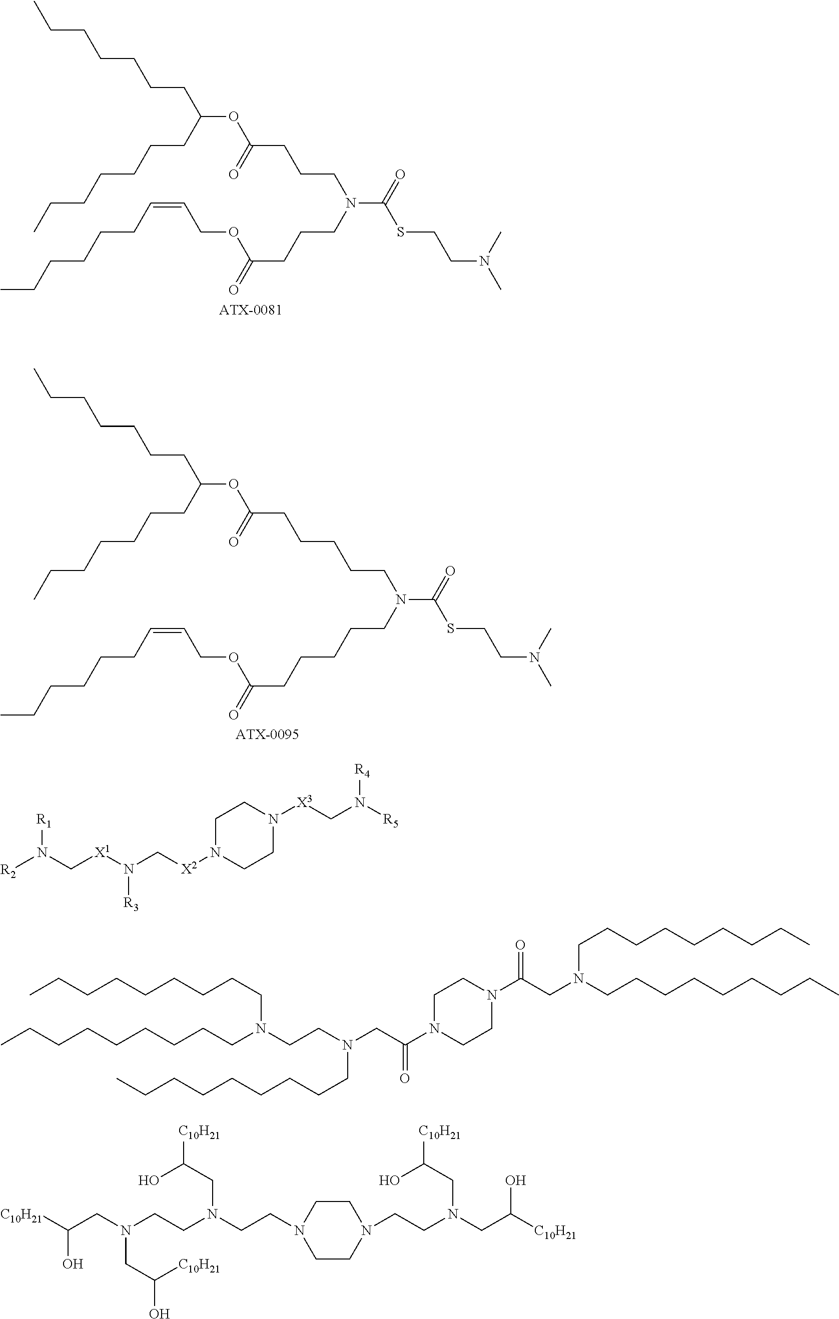

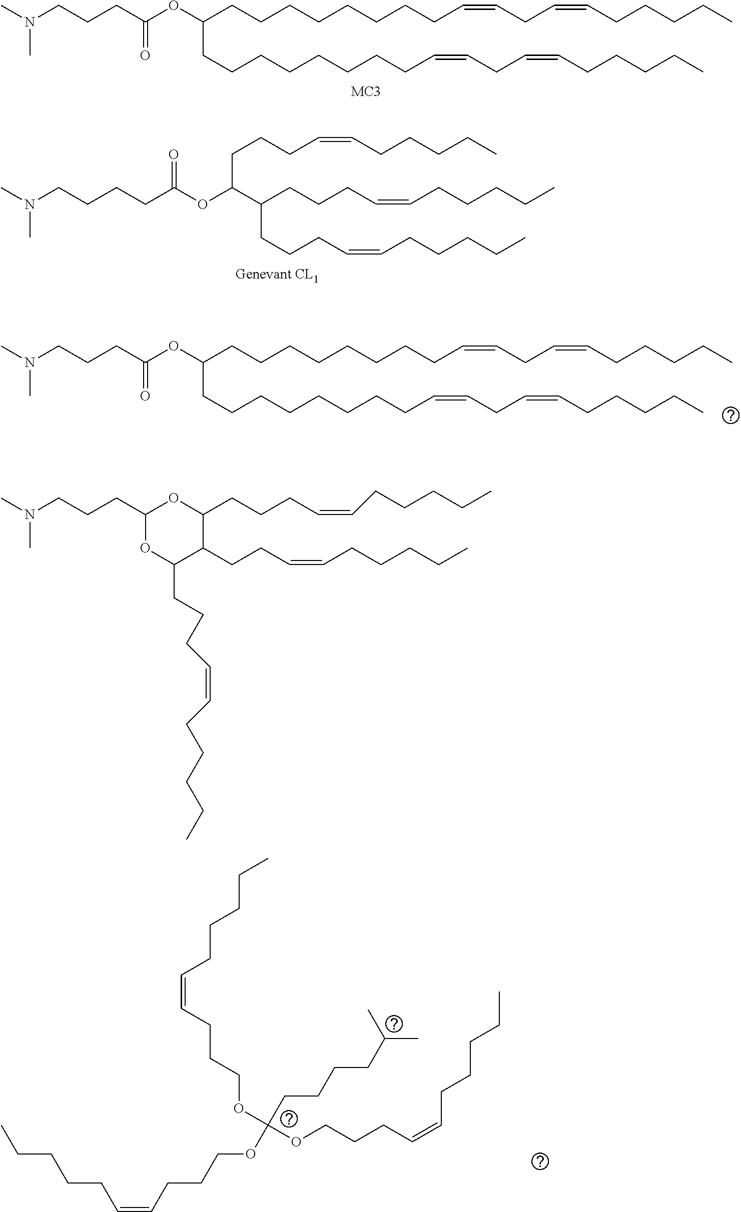

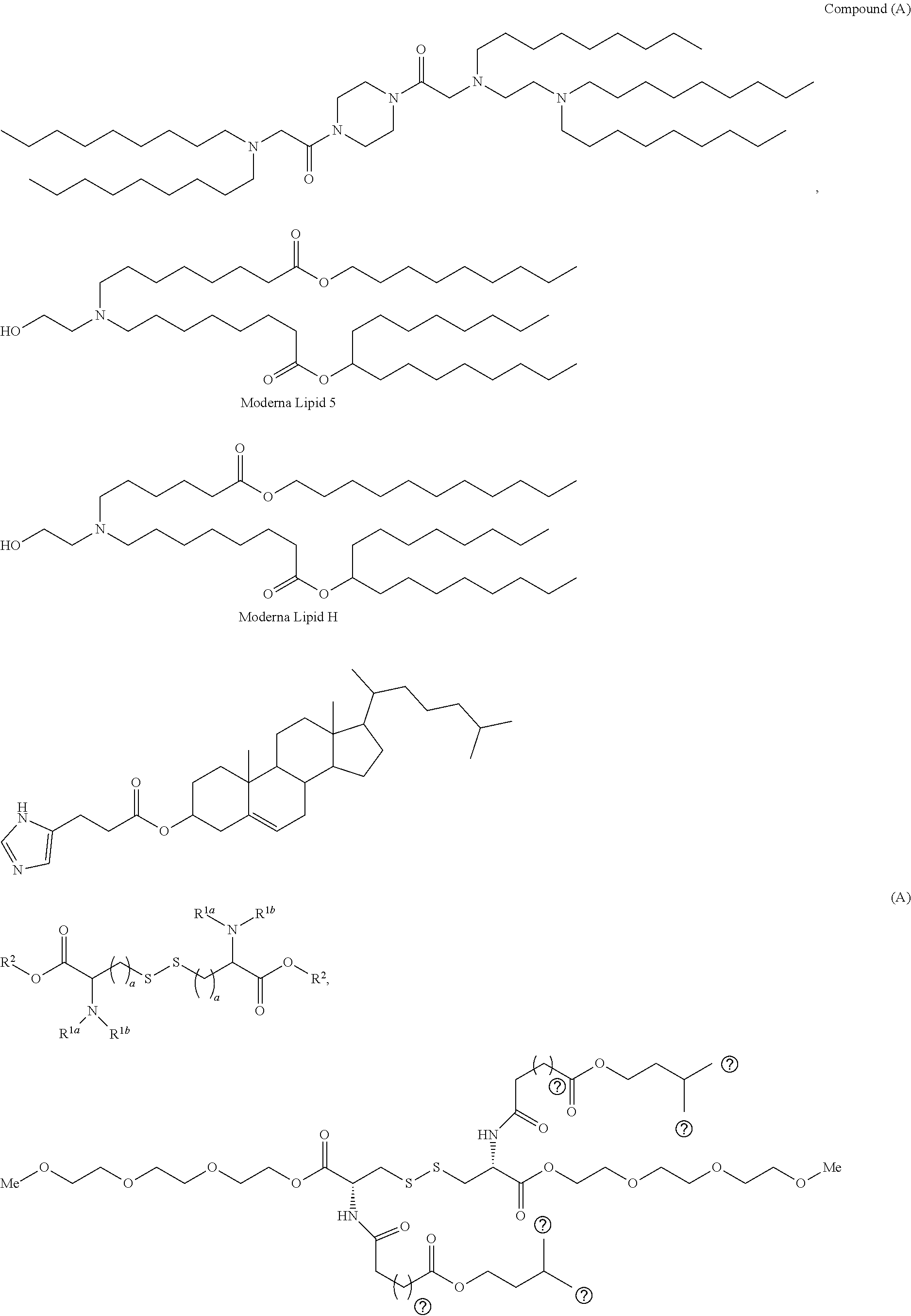

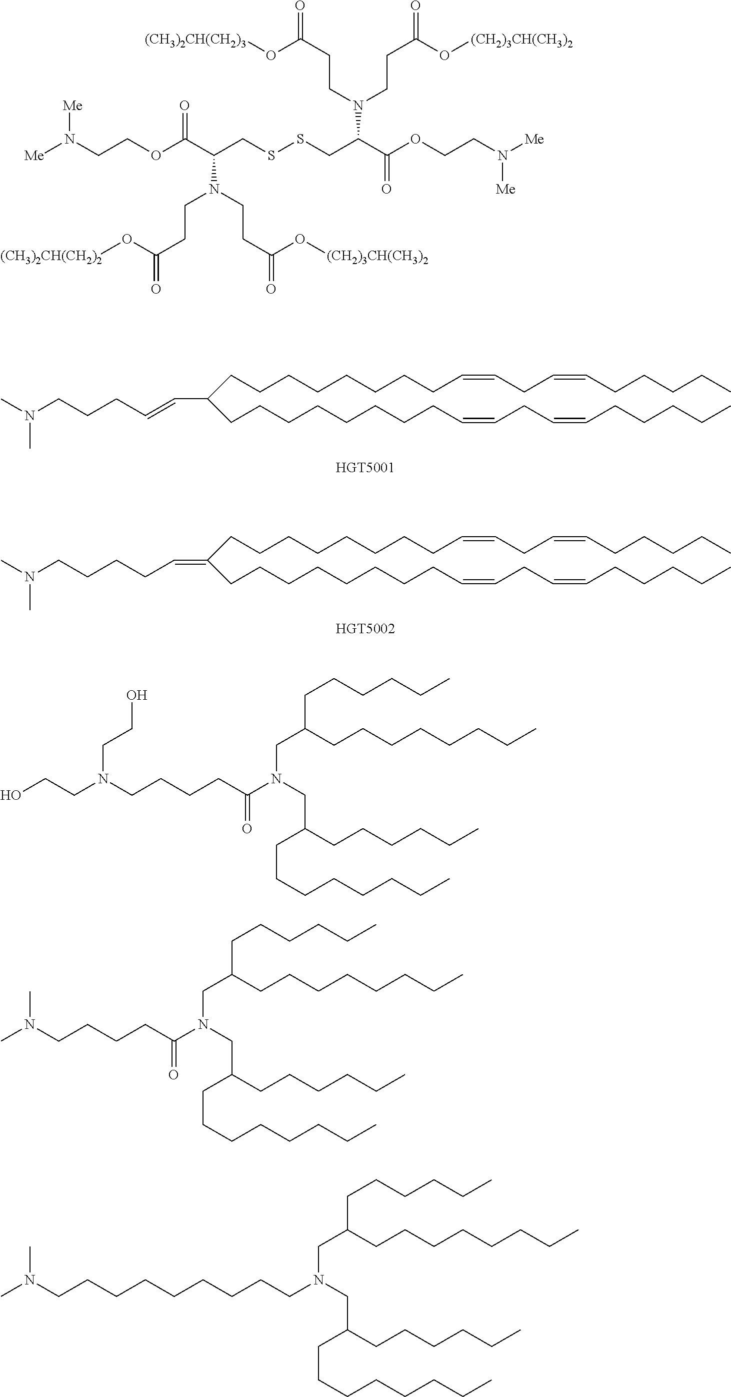

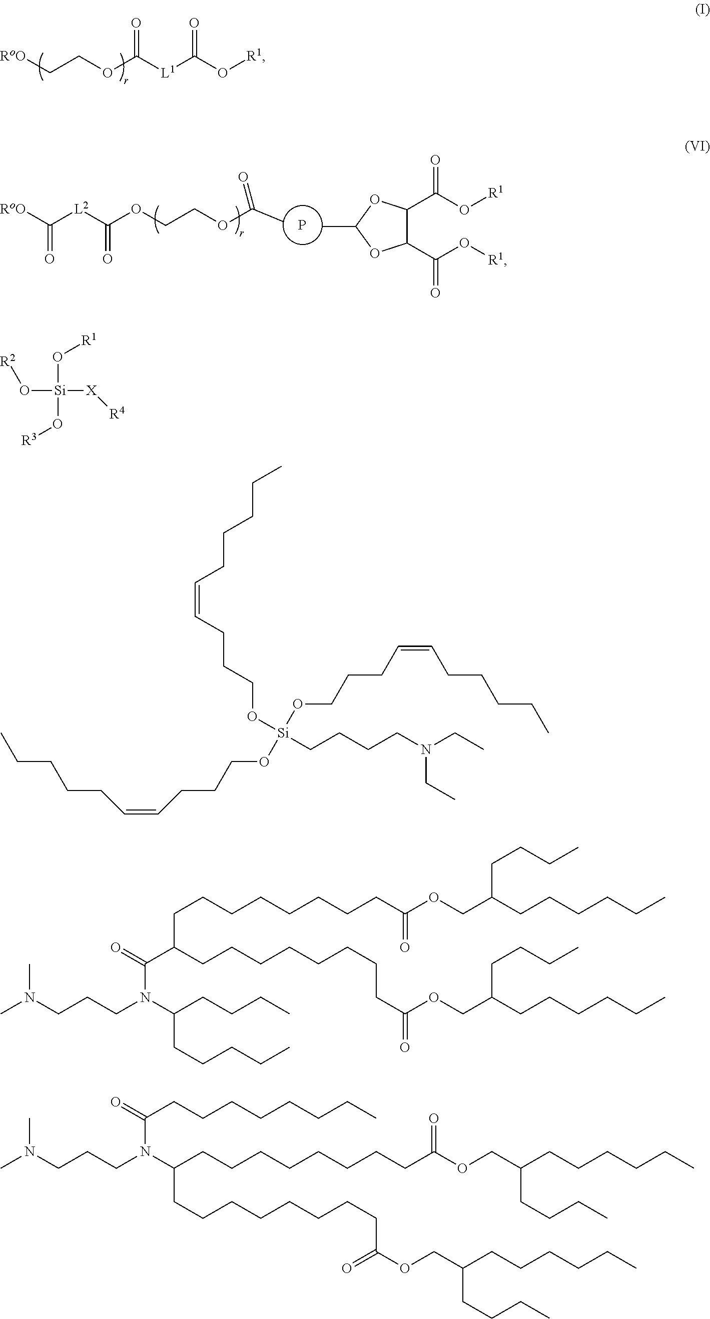

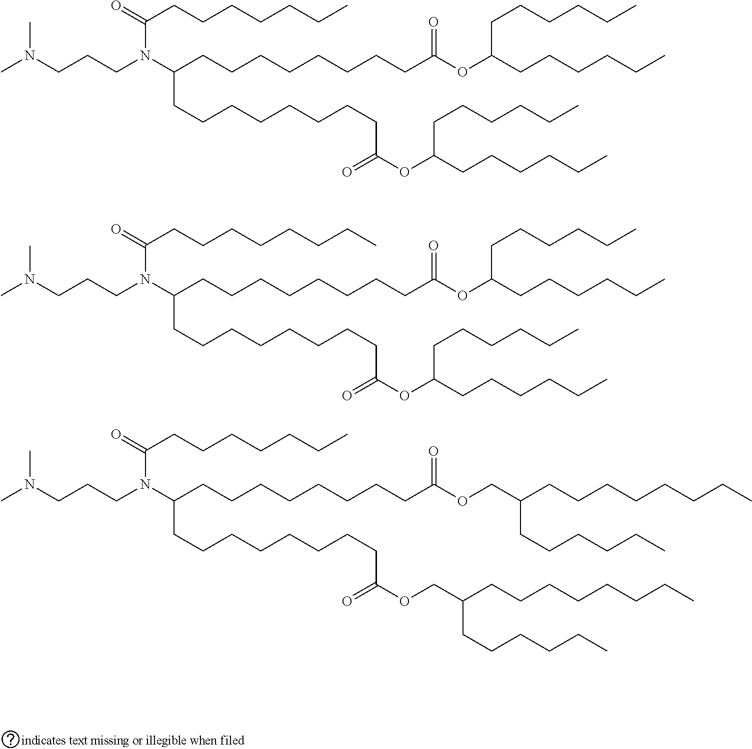

### Neutral Lipids/Phospholipids

In some embodiments, the lipid component further comprises neutral lipids including, but not limited to, a phospholipid selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and mixtures thereof.

### Polymer-Conjugated Lipids

In some embodiments, the lipid component further comprises polymer-conjugated lipids, including, but not limited to, a PEGylated lipid selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA or a PEG-DSPE lipid.

Non-exhaustive and non-limiting examples of PEG lipids include:

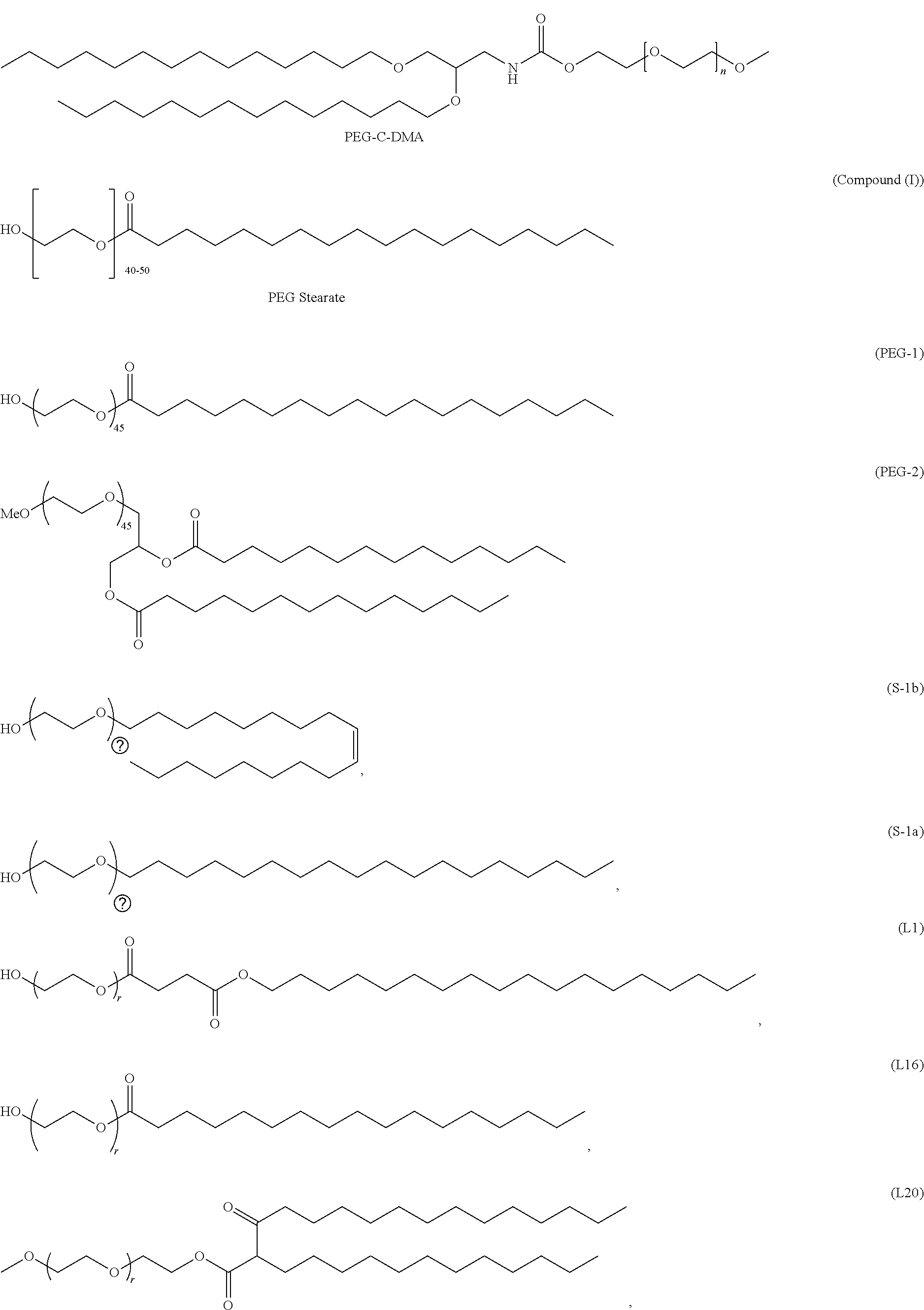

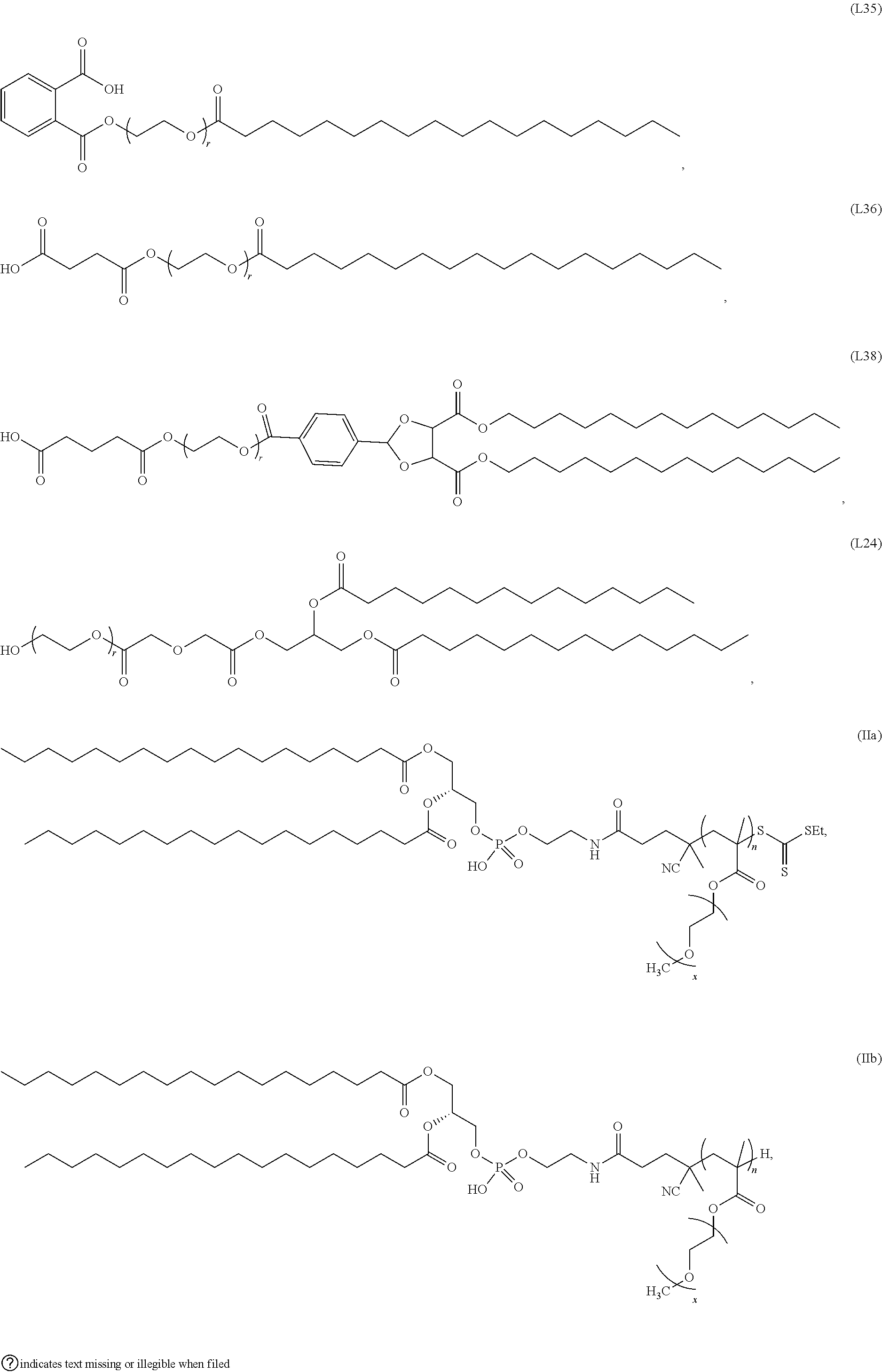

### Structural Lipids/Sterols

In some embodiments, the LNP further comprises a structural component. See generally Patel, S., et al. (2020). Nature Communications, 11(1), 1-13. In some embodiments, the structural component comprises a sterol including, but not limited to, a sterol selected from the group consisting of cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9, 11-dehydroergosterol, tomatidine, tomatine, α-tocopherol, and mixtures thereof. In other embodiments, the structural lipid includes cholesterol and a corticosteroid (e.g., prednisolone, dexamethasone, prednisone, and hydrocortisone), or a combination thereof.

Non-exhaustive and non-limiting examples of structural lipids include.

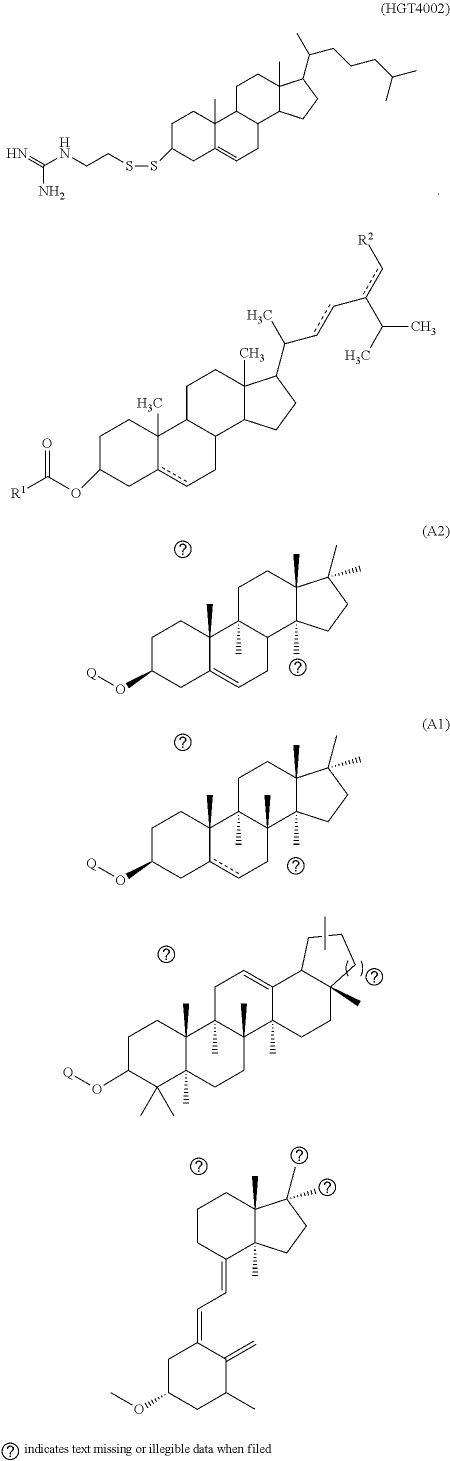

### Formulations

Nanoparticle compositions may include a lipid component and one or more additional components, such as a therapeutic and/or prophylactic. A nanoparticle composition may be designed for one or more specific applications or targets. The elements of a nanoparticle composition may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements.

The lipid component of a nanoparticle composition may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the lipid component of a nanoparticle composition includes an ionizable lipid, a phospholipid, a PEG lipid, and a structural lipid. In certain embodiments, the lipid component of the nanoparticle composition includes about 30 mol % to about 60 mol % ionizable lipid, about 0 mol % to about 30 mol % phospholipid, about 0 mol % to about 10 mol % of PEG lipid, and about 17.5 mol % to about 50 mol % structural lipid, provided that the total mol % does not exceed 100%. In some embodiments, the lipid component of the nanoparticle composition includes about 35 mol % to about 55 mol % compound of ionizable lipid, about 5 mol % to about 25 mol % phospholipid, about 0 mol % to about 10 mol % of PEG lipid, and about 30 mol % to about 40 mol % structural lipid. In a particular embodiment, the lipid component includes about 50 mol % said compound, about 10 mol % phospholipid, about 38.5 mol % structural lipid, and about 1.5 mol % of PEG lipid. In another embodiment, the lipid component includes about 40 mol % said compound, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

In some embodiments, the ionizable lipids comprise between about 20 and about 60 mol % of the lipid component. In other embodiments, the ionizable lipids comprise between about 35 and about 55 mol % of the lipid component. In various embodiments, the ionizable lipids comprise about 20, 21, 22, 23, 24, 25, 26, 27, 28, 29, 30, 35, 40, 45, 50, 55, or 60 mol % of the lipid component.

In some embodiments, the neutral lipids comprise between about 0 and about 30 mol % of the lipid component. In other embodiments, the neutral lipids comprise between about 5 and about 25 mol % of the lipid component. In various embodiments, the neutral lipids comprise about 1, 2, 3, 4, 5, 6, 7, 8, 9, 10, 11, 12, 13, 14, 15, 16, 17, 18, 19, 20, 25, or 30 mol % of the lipid component.

In some embodiments, the polymer-conjugated lipids comprise between about 0 and about 15 mol % of the lipid component. In other embodiments, the polymer-conjugated lipids comprise between about 0.5 and about 10 mol % of the lipid component. In various embodiments, the polymer-conjugated lipids comprise about 0.5, 1, 1.5, 2, 2.5, 3, 3.5, 4, 4.5, 5, 5.5, 6, 6.5, 7, 7.5, 8, 8.5 9, 9.5, 10, or 15 mol % of the lipid component.

In some embodiments, the structural component comprises about 17.5 mol % to about 50 mol % of the lipid component. In other embodiments, the structural component comprises about 30 to about 40 mol % of the lipid component. In various embodiments, the structural component comprises about 17.5, 20, 22.5, 25, 27.5, 30, 31, 32, 33, 34, 35, 36, 37, 38, 39, 40, 41, 42, 43, 44, 45, 46, 47, 48, 49, or 50 mol % of the lipid component.

The structural component may alternatively be expressed as a ratio relative to the lipid component. In some embodiments, the structural component is in a ratio of about 1:1 with the lipid component (sterol:lipids). In other embodiments, the structural component is in a ratio of about 1:5 with the lipid component (sterol:lipids). In various embodiments, the structural component is in a ratio of about 1:1, 1:2, 1:3, 1:4, 1:5, 1:6, 1:7, 1:8, 1:9, 1:10, 1:15, 1:20, or 1:25 with the lipid component (sterol:lipids).

Nanoparticle compositions may be designed for one or more specific applications or targets. For example, a nanoparticle composition may be designed to deliver a therapeutic and/or prophylactic such as an RNA to a particular cell, tissue, organ, or system or group thereof in a mammal's body. Physiochemical properties of nanoparticle compositions may be altered in order to increase selectivity for particular bodily targets. For instance, particle sizes may be adjusted based on the fenestration sizes of different organs. The therapeutic and/or prophylactic included in a nanoparticle composition may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a nanoparticle composition may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian joint.

The amount of a therapeutic and/or prophylactic in a nanoparticle composition may depend on the size, composition, desired target and/or application, or other properties of the nanoparticle composition as well as on the properties of the therapeutic and/or prophylactic. For example, the amount of an RNA useful in a nanoparticle composition may depend on the size, sequence, and other characteristics of the RNA. The relative amounts of a therapeutic and/or prophylactic and other elements (e.g., lipids) in a nanoparticle composition may also vary. In some embodiments, the wt/wt ratio of the lipid component to a therapeutic and/or prophylactic in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a nanoparticle composition may, for example, be measured using absorption spectroscopy (e.g., ultraviolet-visible spectroscopy).

In some embodiments, the therapeutic and/or prophylactic comprises a nucleic acid component. In some embodiments, the nucleic acid component comprises RNA including, but not limited to, RNA selected from the group consisting of messenger RNA (mRNA), CRISPR RNA (crRNA), tracrRNA, single-guide RNA (sgRNA), short interfering RNA (siRNA), antisense oligonucleotides (ASO), and mixtures thereof. In other embodiments, the nucleic acid component comprises DNA including, but not limited to, DNA selected from the group consisting of linear DNA, plasmid DNA, antisense oligonucleotide, and mixtures thereof.

In some embodiments, a nanoparticle composition includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

In some embodiments, the nucleic acid component is comprised of a modified nucleic acid. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

In certain embodiments, the present disclosure comprises methods for treating a joint disease or disorder. In other embodiments, the present disclosure comprises methods for treating osteoarthritis. In some embodiments, the present disclosure comprises methods for treating joint inflammation, the method comprising administering a therapeutically effective amount of a CRISPR-Cas composition encapsulated within or associated with a lipid nanoparticle (LNP), wherein the composition comprises one or more non-naturally occurring polynucleotides encoding a Cas protein and at least one sgRNA. In some embodiments, LNPs are administered intra-articularly.

In certain embodiments, the present disclosure comprises methods for treating fibrosis or scarring. In some embodiments, the fibrosis and/or scarring is postoperative and/or post-surgical fibrosis and/or scarring. In some embodiments, the fibrosis and/or scarring is post-ligament reconstruction. In some embodiments, the fibrosis and/or scarring is post-anterior cruciate ligament (ACL) reconstruction. In some embodiments, the fibrosis and/or scarring is post-autograft anterior cruciate ligament (ACL) reconstruction. In some embodiments, the fibrosis and/or scarring is post-allograft anterior cruciate ligament (ACL) reconstruction. In some embodiments, the fibrosis and/or scarring is due to knee arthrofibrosis. In some embodiments, the fibrosis and/or scarring is due to intra-articular fibrous nodules. In some embodiments, the fibrosis and/or scarring is post-total knee arthroplasty (TKA). In some embodiments, the fibrosis and/or scarring is due to knee arthrofibrosis following TKA. In some embodiments, the fibrosis and/or scarring is post-microdiscectomy. In some embodiments, the fibrosis and/or scarring is due to epidural fibrosis post-microdiscectomy. In some embodiments, the present disclosure comprises methods for treating fibrosis or scarring, the method comprising administering a therapeutically effective amount of a CRISPR-Cas composition encapsulated within or associated with a lipid nanoparticle (LNP), wherein the composition comprises one or more non-naturally occurring polynucleotides encoding a Cas protein and at least one sgRNA. In some embodiments, LNPs are administered locally. In other embodiments, LNPs are administered intra-articularly. In some embodiments, the pharmaceutical composition is administered during a surgery and/or after a surgery.

In certain embodiments, the present disclosure comprises methods for treating low back pain. In other embodiments, the present disclosure comprises methods for treating discogenic disorders. In some embodiments, the present disclosure comprises methods for treating localized nociception, inflammation, or morphological changes associated with back or spine conditions or disorders in a subject in need thereof, the method comprising administering a therapeutically effective amount of a CRISPR-Cas composition encapsulated within or associated with a lipid nanoparticle (LNP), wherein the composition comprises one or more non-naturally occurring polynucleotides encoding a Cas protein and at least one sgRNA. In some embodiments, LNPs are administered intradiscally. In other embodiments, LNPs are administered epidurally.

As used herein, a “lipid component” is that component of a nanoparticle composition that includes one or more lipids. For example, the lipid component may include one or more cationic/ionizable, PEGylated, structural, or other lipids, such as phospholipids.

As used herein, the term “delivering” means providing an entity to a destination. For example, delivering a therapeutic and/or prophylactic to a subject may involve administering a nanoparticle composition including the therapeutic and/or prophylactic to the subject (e.g., by an intravenous, intramuscular, intradermal, subcutaneous, intraarticular, or intradiscal route). Administration of a nanoparticle composition to a mammal or mammalian cell may involve contacting one or more cells with the nanoparticle composition.

As used herein, “naturally occurring” means existing in nature without artificial aid.

As used herein, a “PEG lipid” or “PEGylated lipid” refers to a lipid comprising a polyethylene glycol component. These lipids may also be referred to a PEG-modified lipids.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

### Physical Properties

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvem Instruments Ltd, Malvern, Worcestershire, UK) may also be used to measure multiple characteristics of a nanoparticle composition, such as particle size, polydispersity index, and zeta potential.

The mean size of a nanoparticle composition may be between 10 nm and 1 micrometer, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition may be from about 70 nm to about 100 nm. In a particular embodiment, the mean size may be about 80 nm. In other embodiments, the mean size may be about 100 nm.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic and/or prophylactic in a solution containing the nanoparticle composition before and after breaking up the nanoparticle composition with one or more organic solvents or detergents. Fluorescence may be used to measure the amount of free therapeutic and/or prophylactic (e.g., RNA) in a solution. For the nanoparticle compositions described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A nanoparticle composition may optionally comprise one or more coatings. For example, a nanoparticle composition may be formulated in a capsule, film, or tablet having a coating. A capsule, film, or tablet including a composition described herein may have any useful size, tensile strength, hardness, or density.

Non-limiting examples of genes that may be silenced or inhibited by permanently gene-editing synoviocytes via a CRISPR method include IL-1α, IL-1β, IL-4, IL-9, IL-10, IL-13, and TNF-α.

Non-limiting examples of genes that may be enhanced by permanently gene-editing synoviocytes via a CRISPR method include IL-1α, IL-1β, IL-4, IL-9, IL-10, IL-13, and TNF-α.

Examples of systems, methods, and compositions for altering the expression of a target gene sequence by a CRISPR method, and which may be used in accordance with embodiments of the present disclosure, are described in U.S. Pat. Nos. 8,697,359; 8,993,233; 8,795,965; 8,771,945; 8,889,356; 8,865,406; 8,999,641; 8,945,839; 8,932,814; 8,871,445; 8,906,616; and 8,895,308, which are incorporated by reference herein. Resources for carrying out CRISPR methods, such as plasmids for expressing CRISPR-Cas9 and CRISPR-Cpf1, are commercially available from companies such as GenScript.

In an embodiment, genetic modifications of at least a portion of a joint's synoviocytes, as described herein, may be performed using the CRISPR-Cpf1 system as described in U.S. Pat. No. 9,790,490, the disclosure of which is incorporated by reference herein.

In an embodiment, genetic modifications of at least a portion of a joint's synoviocytes, as described herein, may be performed using a CRISPR-Cas system comprising single vector systems as described in U.S. Pat. No. 9,907,863, the disclosure of which is incorporated by reference herein. TALE Methods

A pharmaceutical composition for the treatment or prevention of a joint disease or condition comprising a gene-editing system, wherein said gene-editing system targets at least one locus related to joint function, wherein the method further comprises gene-editing at least a portion of joint synoviocytes by a TALE method. According to particular embodiments, the use of a TALE method to target at least one locus related to joint function, wherein the gene-editing at least a portion of a joint's synoviocytes. Alternatively, the use of a TALE method during to target at least one locus related to joint function, wherein the gene-editing at least a portion of a joint's synoviocytes to cause expression of at least one locus related to joint function genes to be enhanced in at least a portion of the joint synoviocytes.

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Cellectis Bioresearch (Paris, France), Transposagen Biopharmaceuticals (Lexington, Ky., USA), and Life Technologies (Grand Island, N.Y., USA). TALE and TALEN methods suitable for use in the present disclosure are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein.

Non-limiting examples of genes that may be silenced or inhibited by permanently gene-editing synoviocytes via a TALE method include IL-1α, IL-1β, IL-4, IL-9, IL-10, IL-13, and TNF-α.

Non-limiting examples of genes that may be enhanced by permanently gene-editing synoviocytes via a TALE method include IL-1α, IL-1β, IL-4, IL-9, IL-10, IL-13, and TNF-α.

Examples of systems, methods, and compositions for altering the expression of a target gene sequence by a TALE method, and which may be used in accordance with embodiments of the present disclosure, are described in U.S. Pat. No. 8,586,526, which is incorporated by reference herein.

### Zinc Finger Methods

A pharmaceutical composition for the treatment or prevention of a joint disease or condition comprising a gene-editing system, wherein said gene-editing system targets at least one locus related to joint function, wherein the method further comprises gene-editing at least a portion of joint synoviocytes by a zinc finger or zinc finger nuclease method. According to particular embodiments, the use of a zinc finger method to target at least one locus related to joint function, wherein the gene-editing at least a portion of a joint's synoviocytes. Alternatively, the use of a zinc finger method during to target at least one locus related to joint function, wherein the gene-editing at least a portion of a joint's synoviocytes to cause expression of at least one locus related to joint function genes to be enhanced in at least a portion of the joint synoviocytes.

An individual zinc finger contains approximately 30 amino acids in a conserved ββα configuration. Several amino acids on the surface of the α-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially; Sangamo Biosciences (Richmond, Calif., USA) has developed a propriety platform (CompoZr®) for zinc-finger construction in partnership with Sigma-Aldrich (St. Louis, Mo., USA).

Non-limiting examples of genes that may be silenced or inhibited by permanently gene-editing synoviocytes via a zinc finger method include IL-1α, IL-1β, IL-4, IL-9, IL-10, IL-13, TNF-α. IL-6, IL-8, IL-18, a matrix metalloproteinase (MMP), or a component of the NLRP3 inflammasome. In some embodiments, the component of the NLRP3 inflammasome comprises NLRP3, ASC (apoptosis-associated speck-like protein containing a CARD), caspase-1, and combinations thereof.

Non-limiting examples of genes that may be enhanced by permanently gene-editing synoviocytes via a zinc finger method include group comprising IL-1Ra, TIMP-1, TIMP-2, TIMP-3, TIMP-4, and combinations thereof. In an aspect, the disclosure provides compositions for up-regulation of anti-inflammatory cytokines.

Examples of systems, methods, and compositions for altering the expression of a target gene sequence by a zinc finger method, which may be used in accordance with embodiments of the present disclosure, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, which are incorporated by reference herein.

In some aspects, cells may be gene-edited ex vivo, wherein the gene-editing targets one or more anti-inflammatory cytokine locus. In some aspects, the cells are non-synovial cells. In some aspects, the cells are mesenchymal stem cells. In some aspect, the cells are macrophages. In some aspects, the present disclosure provides for a pharmaceutical composition for the treatment or prevention of a joint disease or condition comprising a population of gene-edited cells, wherein said gene-edited cells are edited by a gene-editing system targeting at least one locus related to joint function. In an aspect, the population of gene-edited cells are injected into a synovial joint.

Other examples of systems, methods, and compositions for altering the expression of a target gene sequence by a zinc finger method, which may be used in accordance with embodiments of the present disclosure, are described in Beane, et al., Mol. Therapy, 2015, 23 1380-1390, the disclosure of which is incorporated by reference herein.

### EXAMPLE EMBODIMENTS

In some embodiments, the present disclosure provides a pharmaceutical composition for the treatment or prevention of a joint disease or condition, the composition including a therapeutically effective amount of one or more nucleic acids encoding a Clustered Regularly Interspaced Short Palindromic Repeats (CRISPR) gene-editing system. The system includes a CRISPR Associated Protein 9 (Cas9) protein, and at least one guide RNA targeting an IL-1α or IL-1β gene, wherein the target sequence is adjacent to a protospacer adjacent motif (PAM) sequence for the Cas9 protein.

In some embodiments, the at least one guide RNA targets a human IL-1α gene, and includes a crRNA sequence that is complementary to a target sequence in exon 2 of the IL-1α gene. In some embodiments, the crRNA sequence forms no more than 5 mismatches with the target sequence in exon 2 of the human IL-1α gene. In some embodiments, the crRNA sequence forms no more than 4 mismatches with the target sequence in exon 2 of the human IL-1α gene. In some embodiments, the crRNA sequence forms no more than 3 mismatches with the target sequence in exon 2 of the human IL-1α gene. In some embodiments, the crRNA sequence forms no more than 2 mismatches with the target sequence in exon 2 of the human IL-1α gene. In some embodiments, the crRNA sequence forms no more than 1 mismatch with the target sequence in exon 2 of the human IL-1α gene. In some embodiments, the crRNA sequence forms no mismatches with the target sequence in exon 2 of the human IL-1α gene.

In some embodiments, the at least one guide RNA targets a human IL-1α gene, and includes a crRNA sequence that is complementary to a target sequence in exon 3 of the IL-1α gene. In some embodiments, the crRNA sequence forms no more than 5 mismatches with the target sequence in exon 3 of the human IL-1α gene. In some embodiments, the crRNA sequence forms no more than 4 mismatches with the target sequence in exon 3 of the human IL-1α gene. In some embodiments, the crRNA sequence forms no more than 3 mismatches with the target sequence in exon 3 of the human IL-1α gene. In some embodiments, the crRNA sequence forms no more than 2 mismatches with the target sequence in exon 3 of the human IL-1α gene. In some embodiments, the crRNA sequence forms no more than 1 mismatch with the target sequence in exon 3 of the human IL-1α gene. In some embodiments, the crRNA sequence forms no mismatches with the target sequence in exon 3 of the human IL-1α gene.

In some embodiments, the at least one guide RNA targets a human IL-1α gene, and includes a crRNA sequence that is complementary to a target sequence in exon 4 of the IL-1α gene. In some embodiments, the crRNA sequence forms no more than 5 mismatches with the target sequence in exon 4 of the human IL-1α gene. In some embodiments, the crRNA sequence forms no more than 4 mismatches with the target sequence in exon 4 of the human IL-1α gene. In some embodiments, the crRNA sequence forms no more than 3 mismatches with the target sequence in exon 4 of the human IL-1α gene. In some embodiments, the crRNA sequence forms no more than 2 mismatches with the target sequence in exon 4 of the human IL-1α gene. In some embodiments, the crRNA sequence forms no more than 1 mismatch with the target sequence in exon 4 of the human IL-1α gene. In some embodiments, the crRNA sequence forms no mismatches with the target sequence in exon 4 of the human IL-1α gene.

In some embodiments, the at least one guide RNA targets a human IL-1α gene, and includes a crRNA sequence that is complementary to a target sequence in exon 5 of the IL-1α gene. In some embodiments, the crRNA sequence forms no more than 5 mismatches with the target sequence in exon 5 of the human IL-1α gene. In some embodiments, the crRNA sequence forms no more than 4 mismatches with the target sequence in exon 5 of the human IL-1α gene. In some embodiments, the crRNA sequence forms no more than 3 mismatches with the target sequence in exon 5 of the human IL-1α gene. In some embodiments, the crRNA sequence forms no more than 2 mismatches with the target sequence in exon 5 of the human IL-1α gene. In some embodiments, the crRNA sequence forms no more than 1 mismatch with the target sequence in exon 5 of the human IL-1α gene. In some embodiments, the crRNA sequence forms no mismatches with the target sequence in exon 5 of the human IL-1α gene.

In some embodiments, the at least one guide RNA targets a human IL-1α gene, and includes a crRNA sequence that is complementary to a target sequence in exon 6 of the IL-1α gene. In some embodiments, the crRNA sequence forms no more than 5 mismatches with the target sequence in exon 6 of the human IL-1α gene. In some embodiments, the crRNA sequence forms no more than 4 mismatches with the target sequence in exon 6 of the human IL-1α gene. In some embodiments, the crRNA sequence forms no more than 3 mismatches with the target sequence in exon 6 of the human IL-1α gene. In some embodiments, the crRNA sequence forms no more than 2 mismatches with the target sequence in exon 6 of the human IL-1α gene. In some embodiments, the crRNA sequence forms no more than 1 mismatch with the target sequence in exon 6 of the human IL-1α gene. In some embodiments, the crRNA sequence forms no mismatches with the target sequence in exon 6 of the human IL-1α gene.

In some embodiments, the at least one guide RNA targets a human IL-1α gene, and includes a crRNA sequence that is complementary to a target sequence in exon 7 of the IL-1α gene. In some embodiments, the crRNA sequence forms no more than 5 mismatches with the target sequence in exon 7 of the human IL-1α gene. In some embodiments, the crRNA sequence forms no more than 4 mismatches with the target sequence in exon 7 of the human IL-1α gene. In some embodiments, the crRNA sequence forms no more than 3 mismatches with the target sequence in exon 7 of the human IL-1α gene. In some embodiments, the crRNA sequence forms no more than 2 mismatches with the target sequence in exon 7 of the human IL-1α gene. In some embodiments, the crRNA sequence forms no more than 1 mismatch with the target sequence in exon 7 of the human IL-1α gene. In some embodiments, the crRNA sequence forms no mismatches with the target sequence in exon 7 of the human IL-1α gene.

In some embodiments, the at least one guide RNA targets a human IL-1α gene, and includes a crRNA sequence that is complementary to a target sequence in exon 2 or exon 3 of the IL-1α gene. In some embodiments, the at least one guide RNA targets a human IL-1α gene, and includes a crRNA sequence that is complementary to a target sequence in exon 2 or exon 4 of the IL-1α gene. In some embodiments, the at least one guide RNA targets a human IL-1α gene, and includes a crRNA sequence that is complementary to a target sequence in exon 2 or exon 5 of the IL-1α gene. In some embodiments, the at least one guide RNA targets a human IL-1α gene, and includes a crRNA sequence that is complementary to a target sequence in exon 2 or exon 6 of the IL-1α gene. In some embodiments, the at least one guide RNA targets a human IL-1α gene, and includes a crRNA sequence that is complementary to a target sequence in exon 2 or exon 7 of the IL-1α gene.

In some embodiments, the at least one guide RNA targets a human IL-1α gene, and includes a crRNA sequence that is complementary to a target sequence in exon 3 or exon 4 of the IL-1α gene. In some embodiments, the at least one guide RNA targets a human IL-1α gene, and includes a crRNA sequence that is complementary to a target sequence in exon 3 or exon 5 of the IL-1α gene. In some embodiments, the at least one guide RNA targets a human IL-1α gene, and includes a crRNA sequence that is complementary to a target sequence in exon 3 or exon 6 of the IL-1α gene. In some embodiments, the at least one guide RNA targets a human IL-1α gene, and includes a crRNA sequence that is complementary to a target sequence in exon 3 or exon 7 of the IL-1α gene.

In some embodiments, the at least one guide RNA targets a human IL-1α gene, and includes a crRNA sequence that is complementary to a target sequence in exon 4 or exon 5 of the IL-1α gene. In some embodiments, the at least one guide RNA targets a human IL-1α gene, and includes a crRNA sequence that is complementary to a target sequence in exon 4 or exon 6 of the IL-1α gene. In some embodiments, the at least one guide RNA targets a human IL-1α gene, and includes a crRNA sequence that is complementary to a target sequence in exon 4 or exon 7 of the IL-1α gene.

In some embodiments, the at least one guide RNA targets a human IL-1α gene, and includes a crRNA sequence that is complementary to a target sequence in exon 5 or exon 6 of the IL-1α gene. In some embodiments, the at least one guide RNA targets a human IL-1α gene, and includes a crRNA sequence that is complementary to a target sequence in exon 5 or exon 7 of the IL-1α gene. In some embodiments, the at least one guide RNA targets a human IL-1α gene, and includes a crRNA sequence that is complementary to a target sequence in exon 6 or exon 7 of the IL-1α gene.

In some embodiments, the at least one guide RNA targets a human IL-1α gene, and includes a crRNA sequence that is complementary to a target sequence in exon 2, exon 3, or exon 4 of the IL-1α gene. In some embodiments, the at least one guide RNA targets a human IL-1α gene, and includes a crRNA sequence that is complementary to a target sequence in exon 2, exon 3, or exon 5 of the IL-1α gene. In some embodiments, the at least one guide RNA targets a human IL-1α gene, and includes a crRNA sequence that is complementary to a target sequence in exon 2, exon 3, or exon 6 of the IL-1α gene. In some embodiments, the at least one guide RNA targets a human IL-1α gene, and includes a crRNA sequence that is complementary to a target sequence in exon 2, exon 3, or exon 7 of the IL-1α gene. In some embodiments, the at least one guide RNA targets a human IL-1α gene, and includes a crRNA sequence that is complementary to a target sequence in exon 2, exon 4, or exon 5 of the IL-1α gene. In some embodiments, the at least one guide RNA targets a human IL-1α gene, and includes a crRNA sequence that is complementary to a target sequence in exon 2, exon 4, or exon 6 of the IL-1α gene. In some embodiments, the at least one guide RNA targets a human IL-1α gene, and includes a crRNA sequence that is complementary to a target sequence in exon 2, exon 4, or exon 7 of the IL-1α gene. In some embodiments, the at least one guide RNA targets a human IL-1α gene, and includes a crRNA sequence that is complementary to a target sequence in exon 2, exon 5, or exon 6 of the IL-1α gene. In some embodiments, the at least one guide RNA targets a human IL-1α gene, and includes a crRNA sequence that is complementary to a target sequence in exon 2, exon 5, or exon 7 of the IL-1α gene. In some embodiments, the at least one guide RNA targets a human IL-1α gene, and includes a crRNA sequence that is complementary to a target sequence in exon 2, exon 6, or exon 7 of the IL-1α gene.

In some embodiments, the at least one guide RNA targets a human IL-1α gene, and includes a crRNA sequence that is complementary to a target sequence in exon 3, exon 4, or exon 5 of the IL-1α gene. In some embodiments, the at least one guide RNA targets a human IL-1α gene, and includes a crRNA sequence that is complementary to a target sequence in exon 3, exon 4, or exon 6 of the IL-1α gene. In some embodiments, the at least one guide RNA targets a human IL-1α gene, and includes a crRNA sequence that is complementary to a target sequence in exon 3, exon 4, or exon 7 of the IL-1α gene. In some embodiments, the at least one guide RNA targets a human IL-1α gene, and includes a crRNA sequence that is complementary to a target sequence in exon 3, exon 5, or exon 6 of the IL-1α gene. In some embodiments, the at least one guide RNA targets a human IL-1α gene, and includes a crRNA sequence that is complementary to a target sequence in exon 3, exon 5, or exon 7 of the IL-1α gene. In some embodiments, the at least one guide RNA targets a human IL-1α gene, and includes a crRNA sequence that is complementary to a target sequence in exon 3, exon 6, or exon 7 of the IL-1α gene.

In some embodiments, the at least one guide RNA targets a human IL-1α gene, and includes a crRNA sequence that is complementary to a target sequence in exon 4, exon 5, or exon 6 of the IL-1α gene. In some embodiments, the at least one guide RNA targets a human IL-1α gene, and includes a crRNA sequence that is complementary to a target sequence in exon 4, exon 5, or exon 7 of the IL-1α gene. In some embodiments, the at least one guide RNA targets a human IL-1α gene, and includes a crRNA sequence that is complementary to a target sequence in exon 4, exon 6, or exon 7 of the IL-1α gene. In some embodiments, the at least one guide RNA targets a human IL-1α gene, and includes a crRNA sequence that is complementary to a target sequence in exon 5, exon 6, or exon 7 of the IL-1α gene.

In some embodiments, the at least one guide RNA targets a human IL-1α gene, and includes a crRNA sequence that is complementary to a target sequence in exon 2, exon 3, exon 4, or exon 5 of the IL-1α gene. In some embodiments, the at least one guide RNA targets a human IL-1α gene, and includes a crRNA sequence that is complementary to a target sequence in exon 2, exon 3, exon 4, or exon 6 of the IL-1α gene. In some embodiments, the at least one guide RNA targets a human IL-1α gene, and includes a crRNA sequence that is complementary to a target sequence in exon 2, exon 3, exon 4, or exon 7 of the IL-1α gene. In some embodiments, the at least one guide RNA targets a human IL-1α gene, and includes a crRNA sequence that is complementary to a target sequence in exon 2, exon 4, exon 5, or exon 6 of the IL-1α gene. In some embodiments, the at least one guide RNA targets a human IL-1α gene, and includes a crRNA sequence that is complementary to a target sequence in exon 2, exon 4, exon 5, or exon 7 of the IL-1α gene. In some embodiments, the at least one guide RNA targets a human IL-1α gene, and includes a crRNA sequence that is complementary to a target sequence in exon 2, exon 4, exon 6, or exon 7 of the IL-1α gene. In some embodiments, the at least one guide RNA targets a human IL-1α gene, and includes a crRNA sequence that is complementary to a target sequence in exon 2, exon 5, exon 6, or exon 7 of the IL-1α gene.

In some embodiments, the at least one guide RNA targets a human IL-1α gene, and includes a crRNA sequence that is complementary to a target sequence in exon 3, exon 4, exon 5, or exon 6 of the IL-1α gene. In some embodiments, the at least one guide RNA targets a human IL-1α gene, and includes a crRNA sequence that is complementary to a target sequence in exon 3, exon 4, exon 5, or exon 7 of the IL-1α gene. In some embodiments, the at least one guide RNA targets a human IL-1α gene, and includes a crRNA sequence that is complementary to a target sequence in exon 3, exon 4, exon 6, or exon 7 of the IL-1α gene. In some embodiments, the at least one guide RNA targets a human IL-1α gene, and includes a crRNA sequence that is complementary to a target sequence in exon 3, exon 5, exon 6, or exon 7 of the IL-1α gene. In some embodiments, the at least one guide RNA targets a human IL-1α gene, and includes a crRNA sequence that is complementary to a target sequence in exon 4, exon 5, exon 6, or exon 7 of the IL-1α gene.

In some embodiments, the at least one guide RNA targets a human IL-1α gene, and includes a crRNA sequence that is complementary to a target sequence in exon 2, exon 3, exon 4, exon 5, or exon 6 of the IL-1α gene. In some embodiments, the at least one guide RNA targets a human IL-1α gene, and includes a crRNA sequence that is complementary to a target sequence in exon 2, exon 3, exon 4, exon 5, or exon 7 of the IL-1α gene. In some embodiments, the at least one guide RNA targets a human IL-1α gene, and includes a crRNA sequence that is complementary to a target sequence in exon 3, exon 4, exon 5, exon 6, or exon 7 of the IL-1α gene. In some embodiments, the at least one guide RNA targets a human IL-1α gene, and includes a crRNA sequence that is complementary to a target sequence in exon 2, exon 4, exon 5, exon 6, or exon 7 of the IL-1α gene. In some embodiments, the at least one guide RNA targets a human IL-1α gene, and includes a crRNA sequence that is complementary to a target sequence in exon 2, exon 3, exon 5, exon 6, or exon 6 of the IL-1α gene. In some embodiments, the at least one guide RNA targets a human IL-1α gene, and includes a crRNA sequence that is complementary to a target sequence in exon 2, exon 3, exon 4, exon 6, or exon 7 of the IL-1α gene. In some embodiments, the at least one guide RNA targets a human IL-1α gene, and includes a crRNA sequence that is complementary to a target sequence in exon 2, exon 3, exon 4, exon 5, exon 6, or exon 7 of the IL-1α gene.

In some embodiments, the at least one guide RNA targets a human IL-1α gene, and includes a crRNA sequence having at least 75% identity to a sequence selected from the group consisting of SEQ ID NOs: 168-187, 298-387, and 681-710. In some embodiments, the crRNA sequence has at least 80% identity to a sequence selected from the group consisting of SEQ ID NOs: 168-187, 298-387, and 681-710. In some embodiments, the crRNA sequence has at least 85% identity to a sequence selected from the group consisting of SEQ ID NOs: 168-187, 298-387, and 681-710. In some embodiments, the crRNA sequence has at least 90% identity to a sequence selected from the group consisting of SEQ ID NOs: 168-187, 298-387, and 681-710. In some embodiments, the crRNA sequence has at least 95% identity to a sequence selected from the group consisting of SEQ ID NOs: 168-187, 298-387, and 681-710. In some embodiments, the crRNA sequence is selected from the group consisting of SEQ ID NOs: 168-187, 298-387, and 681-710.

In some embodiments, the at least one guide RNA targets a human IL-1α gene, and includes a crRNA sequence having at least 75% identity to SEQ ID NO:301. In some embodiments, the crRNA sequence has at least 80% identity to SEQ ID NO:301. In some embodiments, the crRNA sequence has at least 85% identity to SEQ ID NO:301. In some embodiments, the crRNA sequence has at least 90% identity to SEQ ID NO:301. In some embodiments, the crRNA sequence has at least 95% identity to SEQ ID NO:301. In some embodiments, the crRNA sequence is SEQ ID NO:301.

In some embodiments, the at least one guide RNA targets a human IL-1α gene, and includes a crRNA sequence having at least 75% identity to SEQ ID NO:309. In some embodiments, the crRNA sequence has at least 80% identity to SEQ ID NO:309. In some embodiments, the crRNA sequence has at least 85% identity to SEQ ID NO:309. In some embodiments, the crRNA sequence has at least 90% identity to SEQ ID NO:309. In some embodiments, the crRNA sequence has at least 95% identity to SEQ ID NO:309. In some embodiments, the crRNA sequence is SEQ ID NO:309.

In some embodiments, the at least one guide RNA targets a human IL-1β gene, and includes a crRNA sequence having at least 75% identity to a sequence selected from the group consisting of SEQ ID NOs: 188-201, 388-496, and 711-740. In some embodiments, the crRNA sequence has at least 80% identity to a sequence selected from the group consisting of SEQ ID NOs: 188-201, 388-496, and 711-740. In some embodiments, the crRNA sequence has at least 85% identity to a sequence selected from the group consisting of SEQ ID NOs: 188-201, 388-496, and 711-740. In some embodiments, the crRNA sequence has at least 90% identity to a sequence selected from the group consisting of SEQ ID NOs: 188-201, 388-496, and 711-740. In some embodiments, the crRNA sequence has at least 95% identity to a sequence selected from the group consisting of SEQ ID NOs: 188-201, 388-496, and 711-740. In some embodiments, the crRNA sequence is selected from the group consisting of SEQ ID NOs: 188-201, 388-496, and 711-740.

In some embodiments, the at least one guide RNA targets a human IL-1β gene, and includes a crRNA sequence that is complementary to a target sequence in exon 2 of the IL-1β gene. In some embodiments, the crRNA sequence forms no more than 5 mismatches with the target sequence in exon 2 of the human IL-1β gene. In some embodiments, the crRNA sequence forms no more than 4 mismatches with the target sequence in exon 2 of the human IL-1β gene. In some embodiments, the crRNA sequence forms no more than 3 mismatches with the target sequence in exon 2 of the human IL-1β gene. In some embodiments, the crRNA sequence forms no more than 2 mismatches with the target sequence in exon 2 of the human IL-1β gene. In some embodiments, the crRNA sequence forms no more than 1 mismatch with the target sequence in exon 2 of the human IL-1β gene. In some embodiments, the crRNA sequence forms no mismatches with the target sequence in exon 2 of the human IL-1β gene.

In some embodiments, the at least one guide RNA targets a human IL-1β gene, and includes a crRNA sequence that is complementary to a target sequence in exon 3 of the IL-1β gene. In some embodiments, the crRNA sequence forms no more than 5 mismatches with the target sequence in exon 3 of the human IL-1β gene. In some embodiments, the crRNA sequence forms no more than 4 mismatches with the target sequence in exon 3 of the human IL-1β gene. In some embodiments, the crRNA sequence forms no more than 3 mismatches with the target sequence in exon 3 of the human IL-1β gene. In some embodiments, the crRNA sequence forms no more than 2 mismatches with the target sequence in exon 3 of the human IL-1β gene. In some embodiments, the crRNA sequence forms no more than 1 mismatch with the target sequence in exon 3 of the human IL-1β gene. In some embodiments, the crRNA sequence forms no mismatches with the target sequence in exon 3 of the human IL-1β gene.

In some embodiments, the at least one guide RNA targets a human IL-1β gene, and includes a crRNA sequence that is complementary to a target sequence in exon 4 of the IL-1β gene. In some embodiments, the crRNA sequence forms no more than 5 mismatches with the target sequence in exon 4 of the human IL-1β gene. In some embodiments, the crRNA sequence forms no more than 4 mismatches with the target sequence in exon 4 of the human IL-1β gene. In some embodiments, the crRNA sequence forms no more than 3 mismatches with the target sequence in exon 4 of the human IL-1β gene. In some embodiments, the crRNA sequence forms no more than 2 mismatches with the target sequence in exon 4 of the human IL-1β gene. In some embodiments, the crRNA sequence forms no more than 1 mismatch with the target sequence in exon 4 of the human IL-1β gene. In some embodiments, the crRNA sequence forms no mismatches with the target sequence in exon 4 of the human IL-1β gene.

In some embodiments, the at least one guide RNA targets a human IL-1β gene, and includes a crRNA sequence that is complementary to a target sequence in exon 5 of the IL-1β gene. In some embodiments, the crRNA sequence forms no more than 5 mismatches with the target sequence in exon 5 of the human IL-1β gene. In some embodiments, the crRNA sequence forms no more than 4 mismatches with the target sequence in exon 5 of the human IL-1β gene. In some embodiments, the crRNA sequence forms no more than 3 mismatches with the target sequence in exon 5 of the human IL-1β gene. In some embodiments, the crRNA sequence forms no more than 2 mismatches with the target sequence in exon 5 of the human IL-1β gene. In some embodiments, the crRNA sequence forms no more than 1 mismatch with the target sequence in exon 5 of the human IL-1β gene. In some embodiments, the crRNA sequence forms no mismatches with the target sequence in exon 5 of the human IL-1β gene.

In some embodiments, the at least one guide RNA targets a human IL-1β gene, and includes a crRNA sequence that is complementary to a target sequence in exon 6 of the IL-1β gene. In some embodiments, the crRNA sequence forms no more than 5 mismatches with the target sequence in exon 6 of the human IL-1β gene. In some embodiments, the crRNA sequence forms no more than 4 mismatches with the target sequence in exon 6 of the human IL-1β gene. In some embodiments, the crRNA sequence forms no more than 3 mismatches with the target sequence in exon 6 of the human IL-1β gene. In some embodiments, the crRNA sequence forms no more than 2 mismatches with the target sequence in exon 6 of the human IL-1β gene. In some embodiments, the crRNA sequence forms no more than 1 mismatch with the target sequence in exon 6 of the human IL-1β gene. In some embodiments, the crRNA sequence forms no mismatches with the target sequence in exon 6 of the human IL-1β gene.

In some embodiments, the at least one guide RNA targets a human IL-1β gene, and includes a crRNA sequence that is complementary to a target sequence in exon 7 of the IL-1β gene. In some embodiments, the crRNA sequence forms no more than 5 mismatches with the target sequence in exon 7 of the human IL-1β gene. In some embodiments, the crRNA sequence forms no more than 4 mismatches with the target sequence in exon 7 of the human IL-1β gene. In some embodiments, the crRNA sequence forms no more than 3 mismatches with the target sequence in exon 7 of the human IL-1β gene. In some embodiments, the crRNA sequence forms no more than 2 mismatches with the target sequence in exon 7 of the human IL-1β gene. In some embodiments, the crRNA sequence forms no more than 1 mismatch with the target sequence in exon 7 of the human IL-1β gene. In some embodiments, the crRNA sequence forms no mismatches with the target sequence in exon 7 of the human IL-1β gene.

In some embodiments, the at least one guide RNA targets a human IL-1β gene, and includes a crRNA sequence that is complementary to a target sequence in exon 2 or exon 3 of the IL-1β gene. In some embodiments, the at least one guide RNA targets a human IL-1β gene, and includes a crRNA sequence that is complementary to a target sequence in exon 2 or exon 4 of the IL-1β gene. In some embodiments, the at least one guide RNA targets a human IL-1β gene, and includes a crRNA sequence that is complementary to a target sequence in exon 2 or exon 5 of the IL-1β gene. In some embodiments, the at least one guide RNA targets a human IL-1β gene, and includes a crRNA sequence that is complementary to a target sequence in exon 2 or exon 6 of the IL-1β gene. In some embodiments, the at least one guide RNA targets a human IL-1β gene, and includes a crRNA sequence that is complementary to a target sequence in exon 2 or exon 7 of the IL-1β gene.

In some embodiments, the at least one guide RNA targets a human IL-1β gene, and includes a crRNA sequence that is complementary to a target sequence in exon 3 or exon 4 of the IL-1β gene. In some embodiments, the at least one guide RNA targets a human IL-1β gene, and includes a crRNA sequence that is complementary to a target sequence in exon 3 or exon 5 of the IL-1β gene. In some embodiments, the at least one guide RNA targets a human IL-1β gene, and includes a crRNA sequence that is complementary to a target sequence in exon 3 or exon 6 of the IL-1β gene. In some embodiments, the at least one guide RNA targets a human IL-1β gene, and includes a crRNA sequence that is complementary to a target sequence in exon 3 or exon 7 of the IL-1β gene.

In some embodiments, the at least one guide RNA targets a human IL-1β gene, and includes a crRNA sequence that is complementary to a target sequence in exon 4 or exon 5 of the IL-1β gene. In some embodiments, the at least one guide RNA targets a human IL-1β gene, and includes a crRNA sequence that is complementary to a target sequence in exon 4 or exon 6 of the IL-1β gene. In some embodiments, the at least one guide RNA targets a human IL-1β gene, and includes a crRNA sequence that is complementary to a target sequence in exon 4 or exon 7 of the IL-1β gene.

In some embodiments, the at least one guide RNA targets a human IL-1β gene, and includes a crRNA sequence that is complementary to a target sequence in exon 5 or exon 6 of the IL-1β gene. In some embodiments, the at least one guide RNA targets a human IL-1β gene, and includes a crRNA sequence that is complementary to a target sequence in exon 5 or exon 7 of the IL-1β gene. In some embodiments, the at least one guide RNA targets a human IL-1β gene, and includes a crRNA sequence that is complementary to a target sequence in exon 6 or exon 7 of the IL-1β gene.

In some embodiments, the at least one guide RNA targets a human IL-1β gene, and includes a crRNA sequence that is complementary to a target sequence in exon 2, exon 3, or exon 4 of the IL-1β gene. In some embodiments, the at least one guide RNA targets a human IL-1β gene, and includes a crRNA sequence that is complementary to a target sequence in exon 2, exon 3, or exon 5 of the IL-1β gene. In some embodiments, the at least one guide RNA targets a human IL-1β gene, and includes a crRNA sequence that is complementary to a target sequence in exon 2, exon 3, or exon 6 of the IL-1β gene. In some embodiments, the at least one guide RNA targets a human IL-1β gene, and includes a crRNA sequence that is complementary to a target sequence in exon 2, exon 3, or exon 7 of the IL-1β gene. In some embodiments, the at least one guide RNA targets a human IL-1β gene, and includes a crRNA sequence that is complementary to a target sequence in exon 2, exon 4, or exon 5 of the IL-1β gene. In some embodiments, the at least one guide RNA targets a human IL-1β gene, and includes a crRNA sequence that is complementary to a target sequence in exon 2, exon 4, or exon 6 of the IL-1β gene. In some embodiments, the at least one guide RNA targets a human IL-1β gene, and includes a crRNA sequence that is complementary to a target sequence in exon 2, exon 4, or exon 7 of the IL-1β gene. In some embodiments, the at least one guide RNA targets a human IL-1β gene, and includes a crRNA sequence that is complementary to a target sequence in exon 2, exon 5, or exon 6 of the IL-1β gene. In some embodiments, the at least one guide RNA targets a human IL-1β gene, and includes a crRNA sequence that is complementary to a target sequence in exon 2, exon 5, or exon 7 of the IL-1β gene. In some embodiments, the at least one guide RNA targets a human IL-1β gene, and includes a crRNA sequence that is complementary to a target sequence in exon 2, exon 6, or exon 7 of the IL-1β gene.

In some embodiments, the at least one guide RNA targets a human IL-1β gene, and includes a crRNA sequence that is complementary to a target sequence in exon 3, exon 4, or exon 5 of the IL-1β gene. In some embodiments, the at least one guide RNA targets a human IL-1β gene, and includes a crRNA sequence that is complementary to a target sequence in exon 3, exon 4, or exon 6 of the IL-1β gene. In some embodiments, the at least one guide RNA targets a human IL-1β gene, and includes a crRNA sequence that is complementary to a target sequence in exon 3, exon 4, or exon 7 of the IL-1β gene. In some embodiments, the at least one guide RNA targets a human IL-1β gene, and includes a crRNA sequence that is complementary to a target sequence in exon 3, exon 5, or exon 6 of the IL-1β gene. In some embodiments, the at least one guide RNA targets a human IL-1β gene, and includes a crRNA sequence that is complementary to a target sequence in exon 3, exon 5, or exon 7 of the IL-1β gene. In some embodiments, the at least one guide RNA targets a human IL-1β gene, and includes a crRNA sequence that is complementary to a target sequence in exon 3, exon 6, or exon 7 of the IL-1β gene.

In some embodiments, the at least one guide RNA targets a human IL-1β gene, and includes a crRNA sequence that is complementary to a target sequence in exon 4, exon 5, or exon 6 of the IL-1β gene. In some embodiments, the at least one guide RNA targets a human IL-1β gene, and includes a crRNA sequence that is complementary to a target sequence in exon 4, exon 5, or exon 7 of the IL-1β gene. In some embodiments, the at least one guide RNA targets a human IL-1β gene, and includes a crRNA sequence that is complementary to a target sequence in exon 4, exon 6, or exon 7 of the IL-1β gene. In some embodiments, the at least one guide RNA targets a human IL-1β gene, and includes a crRNA sequence that is complementary to a target sequence in exon 5, exon 6, or exon 7 of the IL-1β gene.

In some embodiments, the at least one guide RNA targets a human IL-1β gene, and includes a crRNA sequence that is complementary to a target sequence in exon 2, exon 3, exon 4, or exon 5 of the IL-1β gene. In some embodiments, the at least one guide RNA targets a human IL-1β gene, and includes a crRNA sequence that is complementary to a target sequence in exon 2, exon 3, exon 4, or exon 6 of the IL-1β gene. In some embodiments, the at least one guide RNA targets a human IL-1β gene, and includes a crRNA sequence that is complementary to a target sequence in exon 2, exon 3, exon 4, or exon 7 of the IL-1β gene. In some embodiments, the at least one guide RNA targets a human IL-1β gene, and includes a crRNA sequence that is complementary to a target sequence in exon 2, exon 4, exon 5, or exon 6 of the IL-1β gene. In some embodiments, the at least one guide RNA targets a human IL-1β gene, and includes a crRNA sequence that is complementary to a target sequence in exon 2, exon 4, exon 5, or exon 7 of the IL-1β gene. In some embodiments, the at least one guide RNA targets a human IL-1β gene, and includes a crRNA sequence that is complementary to a target sequence in exon 2, exon 4, exon 6, or exon 7 of the IL-1β gene. In some embodiments, the at least one guide RNA targets a human IL-1β gene, and includes a crRNA sequence that is complementary to a target sequence in exon 2, exon 5, exon 6, or exon 7 of the IL-1β gene.

In some embodiments, the at least one guide RNA targets a human IL-1β gene, and includes a crRNA sequence that is complementary to a target sequence in exon 3, exon 4, exon 5, or exon 6 of the IL-1β gene. In some embodiments, the at least one guide RNA targets a human IL-1β gene, and includes a crRNA sequence that is complementary to a target sequence in exon 3, exon 4, exon 5, or exon 7 of the IL-1β gene. In some embodiments, the at least one guide RNA targets a human IL-1β gene, and includes a crRNA sequence that is complementary to a target sequence in exon 3, exon 4, exon 6, or exon 7 of the IL-1β gene. In some embodiments, the at least one guide RNA targets a human IL-1β gene, and includes a crRNA sequence that is complementary to a target sequence in exon 3, exon 5, exon 6, or exon 7 of the IL-1β gene. In some embodiments, the at least one guide RNA targets a human IL-1β gene, and includes a crRNA sequence that is complementary to a target sequence in exon 4, exon 5, exon 6, or exon 7 of the IL-1β gene.

In some embodiments, the at least one guide RNA targets a human IL-1β gene, and includes a crRNA sequence that is complementary to a target sequence in exon 2, exon 3, exon 4, exon 5, or exon 6 of the IL-1β gene. In some embodiments, the at least one guide RNA targets a human IL-1β gene, and includes a crRNA sequence that is complementary to a target sequence in exon 2, exon 3, exon 4, exon 5, or exon 7 of the IL-1β gene. In some embodiments, the at least one guide RNA targets a human IL-1β gene, and includes a crRNA sequence that is complementary to a target sequence in exon 3, exon 4, exon 5, exon 6, or exon 7 of the IL-1β gene. In some embodiments, the at least one guide RNA targets a human IL-1β gene, and includes a crRNA sequence that is complementary to a target sequence in exon 2, exon 4, exon 5, exon 6, or exon 7 of the IL-1β gene. In some embodiments, the at least one guide RNA targets a human IL-1β gene, and includes a crRNA sequence that is complementary to a target sequence in exon 2, exon 3, exon 5, exon 6, or exon 6 of the IL-1β gene. In some embodiments, the at least one guide RNA targets a human IL-1β gene, and includes a crRNA sequence that is complementary to a target sequence in exon 2, exon 3, exon 4, exon 6, or exon 7 of the IL-1β gene. In some embodiments, the at least one guide RNA targets a human IL-1β gene, and includes a crRNA sequence that is complementary to a target sequence in exon 2, exon 3, exon 4, exon 5, exon 6, or exon 7 of the IL-1β gene.

In some embodiments, the at least one guide RNA targets a human IL-1β gene, and includes a crRNA sequence having at least 75% identity to SEQ ID NO:462. In some embodiments, the crRNA sequence has at least 80% identity to SEQ ID NO:462. In some embodiments, the crRNA sequence has at least 85% identity to SEQ ID NO:462. In some embodiments, the crRNA sequence has at least 90% identity to SEQ ID NO:462. In some embodiments, the crRNA sequence has at least 95% identity to SEQ ID NO:462. In some embodiments, the crRNA sequence is SEQ ID NO:462.

In some embodiments, the at least one guide RNA targets a human IL-1β gene, and includes a crRNA sequence having at least 75% identity to SEQ ID NO:391. In some embodiments, the crRNA sequence has at least 80% identity to SEQ ID NO:391. In some embodiments, the crRNA sequence has at least 85% identity to SEQ ID NO:391. In some embodiments, the crRNA sequence has at least 90% identity to SEQ ID NO:391. In some embodiments, the crRNA sequence has at least 95% identity to SEQ ID NO:391. In some embodiments, the crRNA sequence is SEQ ID NO:391.

In some embodiments, the at least one guide RNA targets a human IL-1β gene, and includes a crRNA sequence having at least 75% identity to SEQ ID NO:393. In some embodiments, the crRNA sequence has at least 80% identity to SEQ ID NO:393. In some embodiments, the crRNA sequence has at least 85% identity to SEQ ID NO:393. In some embodiments, the crRNA sequence has at least 90% identity to SEQ ID NO:393. In some embodiments, the crRNA sequence has at least 95% identity to SEQ ID NO:393. In some embodiments, the crRNA sequence is SEQ ID NO:393.

In some embodiments, the at least one guide RNA targets a human IL-1β gene, and includes a crRNA sequence having at least 75% identity to SEQ ID NO:388. In some embodiments, the crRNA sequence has at least 80% identity to SEQ ID NO:388. In some embodiments, the crRNA sequence has at least 85% identity to SEQ ID NO:388. In some embodiments, the crRNA sequence has at least 90% identity to SEQ ID NO:388. In some embodiments, the crRNA sequence has at least 95% identity to SEQ ID NO:388. In some embodiments, the crRNA sequence is SEQ ID NO:388.

In some embodiments, the at least one guide RNA targets a human IL-1β gene, and includes a crRNA sequence having at least 75% identity to SEQ ID NO:389. In some embodiments, the crRNA sequence has at least 80% identity to SEQ ID NO:389. In some embodiments, the crRNA sequence has at least 85% identity to SEQ ID NO:389. In some embodiments, the crRNA sequence has at least 90% identity to SEQ ID NO:389. In some embodiments, the crRNA sequence has at least 95% identity to SEQ ID NO:389. In some embodiments, the crRNA sequence is SEQ ID NO:389.

In some embodiments, the at least one guide RNA targets a canine IL-1α gene, and includes a crRNA sequence having at least 75% identity to a sequence selected from the group consisting of SEQ ID NOs: 202-216, 552-590, and 741-770. In some embodiments, the crRNA sequence has at least 80% identity to a sequence selected from the group consisting of SEQ ID NOs: 202-216, 552-590, and 741-770. In some embodiments, the crRNA sequence has at least 85% identity to a sequence selected from the group consisting of SEQ ID NOs: 202-216, 552-590, and 741-770. In some embodiments, the crRNA sequence has at least 90% identity to a sequence selected from the group consisting of SEQ ID NOs: 202-216, 552-590, and 741-770. In some embodiments, the crRNA sequence has at least 95% identity to a sequence selected from the group consisting of SEQ ID NOs: 202-216, 552-590, and 741-770. In some embodiments, the crRNA sequence is selected from the group consisting of SEQ ID NOs: 202-216, 552-590, and 741-770.

In some embodiments, the at least one guide RNA targets a canine IL-1α gene, and includes a crRNA sequence having at least 75% identity to SEQ ID NO:552. In some embodiments, the crRNA sequence has at least 80% identity to SEQ ID NO:552. In some embodiments, the crRNA sequence has at least 85% identity to SEQ ID NO:552. In some embodiments, the crRNA sequence has at least 90% identity to SEQ ID NO:552. In some embodiments, the crRNA sequence has at least 95% identity to SEQ ID NO:552. In some embodiments, the crRNA sequence is SEQ ID NO:552.

In some embodiments, the at least one guide RNA targets a canine IL-1α gene, and includes a crRNA sequence having at least 75% identity to SEQ ID NO:554. In some embodiments, the crRNA sequence has at least 80% identity to SEQ ID NO:554. In some embodiments, the crRNA sequence has at least 85% identity to SEQ ID NO:554. In some embodiments, the crRNA sequence has at least 90% identity to SEQ ID NO:554. In some embodiments, the crRNA sequence has at least 95% identity to SEQ ID NO:554. In some embodiments, the crRNA sequence is SEQ ID NO:554.

In some embodiments, the at least one guide RNA targets a canine IL-1α gene, and includes a crRNA sequence having at least 75% identity to SEQ ID NO:578. In some embodiments, the crRNA sequence has at least 80% identity to SEQ ID NO:578. In some embodiments, the crRNA sequence has at least 85% identity to SEQ ID NO:578. In some embodiments, the crRNA sequence has at least 90% identity to SEQ ID NO:578. In some embodiments, the crRNA sequence has at least 95% identity to SEQ ID NO:578. In some embodiments, the crRNA sequence is SEQ ID NO:578.

In some embodiments, the at least one guide RNA targets a canine IL-1α gene, and includes a crRNA sequence having at least 75% identity to SEQ ID NO:579. In some embodiments, the crRNA sequence has at least 80% identity to SEQ ID NO:579. In some embodiments, the crRNA sequence has at least 85% identity to SEQ ID NO:579. In some embodiments, the crRNA sequence has at least 90% identity to SEQ ID NO:579. In some embodiments, the crRNA sequence has at least 95% identity to SEQ ID NO:579. In some embodiments, the crRNA sequence is SEQ ID NO:579.

In some embodiments, the at least one guide RNA targets a canine IL-1β gene, and includes a crRNA sequence having at least 75% identity to a sequence selected from the group consisting of SEQ ID NOs: 217-235, 497-551, and 771-800. In some embodiments, the crRNA sequence has at least 80% identity to a sequence selected from the group consisting of SEQ ID NOs: 217-235, 497-551, and 771-800. In some embodiments, the crRNA sequence has at least 85% identity to a sequence selected from the group consisting of SEQ ID NOs: 217-235, 497-551, and 771-800. In some embodiments, the crRNA sequence has at least 90% identity to a sequence selected from the group consisting of SEQ ID NOs: 217-235, 497-551, and 771-800. In some embodiments, the crRNA sequence has at least 95% identity to a sequence selected from the group consisting of SEQ ID NOs: 217-235, 497-551, and 771-800. In some embodiments, the crRNA sequence is selected from the group consisting of SEQ ID NOs: 217-235, 497-551, and 771-800.

In some embodiments, the at least one guide RNA targets a canine IL-1β gene, and includes a crRNA sequence having at least 75% identity to SEQ ID NO:498. In some embodiments, the crRNA sequence has at least 80% identity to SEQ ID NO:498. In some embodiments, the crRNA sequence has at least 85% identity to SEQ ID NO:498. In some embodiments, the crRNA sequence has at least 90% identity to SEQ ID NO:498. In some embodiments, the crRNA sequence has at least 95% identity to SEQ ID NO:498. In some embodiments, the crRNA sequence is SEQ ID NO:498.

In some embodiments, the at least one guide RNA targets a canine IL-1β gene, and includes a crRNA sequence having at least 75% identity to SEQ ID NO:506. In some embodiments, the crRNA sequence has at least 80% identity to SEQ ID NO:506. In some embodiments, the crRNA sequence has at least 85% identity to SEQ ID NO:506. In some embodiments, the crRNA sequence has at least 90% identity to SEQ ID NO:506. In some embodiments, the crRNA sequence has at least 95% identity to SEQ ID NO:506. In some embodiments, the crRNA sequence is SEQ ID NO:506.

In some embodiments, the pharmaceutical composition includes one or more viral vectors, as described herein, collectively comprising the one or more nucleic acids. In some embodiments, the one or more viral vectors include a recombinant virus selected from a retrovirus, an adenovirus, an adeno-associated virus, a lentivirus, and a herpes simplex virus-1. In some embodiments, the one of more viral vectors include a recombinant adeno-associated virus (AAV). In some embodiments, the recombinant AAV is of serotype 5 (AAV5). In some embodiments, the recombinant AAV is of serotype 6 (AAV6).

In some embodiments, the one of more viral vectors include a first viral vector comprising a first nucleic acid, in the one or more nucleic acids, encoding the Cas9 protein, and a second viral vector comprising a second nucleic acid, in the one or more nucleic acids, encoding the at least one guide RNA. In some embodiments, the one of more viral vectors comprise a viral vector comprising a single nucleic acid, wherein the single nucleic acid encodes the Cas9 protein and the at least one guide RNA.

In some embodiments, the composition includes one or more liposomes collectively comprising the one or more nucleic acids. In some embodiments, the one or more nucleic acids are present in a naked state.

In some embodiments, the Cas9 protein is an S. pyogenes Cas9 polypeptide. In some embodiments, the Cas9 protein is an S. aureus Cas9 polypeptide.

In some embodiments, the composition is formulated for parenteral administration. In some embodiments, the composition is formulated for intra-articular injection within a joint of a subject.

In another aspect, the disclosure provides a method for the treatment or prevention of a joint disease or condition in a subject in need thereof. The method includes administering, to a joint of the subject, a pharmaceutical composition comprising a pharmaceutically effective amount of a composition comprising one or more nucleic acids encoding a Clustered Regularly Interspaced Short Palindromic Repeats (CRISPR) gene-editing system. The system includes a CRISPR Associated Protein 9 (Cas9) protein, and at least one guide RNA targeting an IL-1α or IL-1β gene, wherein the target sequence is adjacent to a protospacer adjacent motif (PAM) sequence for the Cas9 protein.

In some embodiments, the at least one guide RNA targets a human IL-1α gene, and includes a crRNA sequence having at least 75% identity to a sequence selected from the group consisting of SEQ ID NOs: 168-187, 298-387, and 681-710. In some embodiments, the crRNA sequence has at least 80% identity to a sequence selected from the group consisting of SEQ ID NOs: 168-187, 298-387, and 681-710. In some embodiments, the crRNA sequence has at least 85% identity to a sequence selected from the group consisting of SEQ ID NOs: 168-187, 298-387, and 681-710. In some embodiments, the crRNA sequence has at least 90% identity to a sequence selected from the group consisting of SEQ ID NOs: 168-187, 298-387, and 681-710. In some embodiments, the crRNA sequence has at least 95% identity to a sequence selected from the group consisting of SEQ ID NOs: 168-187, 298-387, and 681-710. In some embodiments, the crRNA sequence is selected from the group consisting of SEQ ID NOs: 168-187, 298-387, and 681-710.

In some embodiments, the at least one guide RNA targets a human IL-1α gene, and includes a crRNA sequence having at least 75% identity to SEQ ID NO:301. In some embodiments, the crRNA sequence has at least 80% identity to SEQ ID NO:301. In some embodiments, the crRNA sequence has at least 85% identity to SEQ ID NO:301. In some embodiments, the crRNA sequence has at least 90% identity to SEQ ID NO:301. In some embodiments, the crRNA sequence has at least 95% identity to SEQ ID NO:301. In some embodiments, the crRNA sequence is SEQ ID NO:301.

In some embodiments, the at least one guide RNA targets a human IL-1α gene, and includes a crRNA sequence having at least 75% identity to SEQ ID NO:309. In some embodiments, the crRNA sequence has at least 80% identity to SEQ ID NO:309. In some embodiments, the crRNA sequence has at least 85% identity to SEQ ID NO:309. In some embodiments, the crRNA sequence has at least 90% identity to SEQ ID NO:309. In some embodiments, the crRNA sequence has at least 95% identity to SEQ ID NO:309. In some embodiments, the crRNA sequence is SEQ ID NO:309.

In some embodiments, the at least one guide RNA targets a human IL-1β gene, and includes a crRNA sequence having at least 75% identity to a sequence selected from the group consisting of SEQ ID NOs: 188-201, 388-496, and 711-740. In some embodiments, the crRNA sequence has at least 80% identity to a sequence selected from the group consisting of SEQ ID NOs: 188-201, 388-496, and 711-740. In some embodiments, the crRNA sequence has at least 85% identity to a sequence selected from the group consisting of SEQ ID NOs: 188-201, 388-496, and 711-740. In some embodiments, the crRNA sequence has at least 90% identity to a sequence selected from the group consisting of SEQ ID NOs: 188-201, 388-496, and 711-740. In some embodiments, the crRNA sequence has at least 95% identity to a sequence selected from the group consisting of SEQ ID NOs: 188-201, 388-496, and 711-740. In some embodiments, the crRNA sequence is selected from the group consisting of SEQ ID NOs: 188-201, 388-496, and 711-740.

In some embodiments, the at least one guide RNA targets a human IL-1β gene, and includes a crRNA sequence having at least 75% identity to SEQ ID NO:462. In some embodiments, the crRNA sequence has at least 80% identity to SEQ ID NO:462. In some embodiments, the crRNA sequence has at least 85% identity to SEQ ID NO:462. In some embodiments, the crRNA sequence has at least 90% identity to SEQ ID NO:462. In some embodiments, the crRNA sequence has at least 95% identity to SEQ ID NO:462. In some embodiments, the crRNA sequence is SEQ ID NO:462.

In some embodiments, the at least one guide RNA targets a human IL-1β gene, and includes a crRNA sequence having at least 75% identity to SEQ ID NO:391. In some embodiments, the crRNA sequence has at least 80% identity to SEQ ID NO:391. In some embodiments, the crRNA sequence has at least 85% identity to SEQ ID NO:391. In some embodiments, the crRNA sequence has at least 90% identity to SEQ ID NO:391. In some embodiments, the crRNA sequence has at least 95% identity to SEQ ID NO:391. In some embodiments, the crRNA sequence is SEQ ID NO:391.

In some embodiments, the at least one guide RNA targets a human IL-1β gene, and includes a crRNA sequence having at least 75% identity to SEQ ID NO:393. In some embodiments, the crRNA sequence has at least 80% identity to SEQ ID NO:393. In some embodiments, the crRNA sequence has at least 85% identity to SEQ ID NO:393. In some embodiments, the crRNA sequence has at least 90% identity to SEQ ID NO:393. In some embodiments, the crRNA sequence has at least 95% identity to SEQ ID NO:393. In some embodiments, the crRNA sequence is SEQ ID NO:393.

In some embodiments, the at least one guide RNA targets a human IL-1β gene, and includes a crRNA sequence having at least 75% identity to SEQ ID NO:388. In some embodiments, the crRNA sequence has at least 80% identity to SEQ ID NO:388. In some embodiments, the crRNA sequence has at least 85% identity to SEQ ID NO:388. In some embodiments, the crRNA sequence has at least 90% identity to SEQ ID NO:388. In some embodiments, the crRNA sequence has at least 95% identity to SEQ ID NO:388. In some embodiments, the crRNA sequence is SEQ ID NO:388.

In some embodiments, the at least one guide RNA targets a human IL-1β gene, and includes a crRNA sequence having at least 75% identity to SEQ ID NO:389. In some embodiments, the crRNA sequence has at least 80% identity to SEQ ID NO:389. In some embodiments, the crRNA sequence has at least 85% identity to SEQ ID NO:389. In some embodiments, the crRNA sequence has at least 90% identity to SEQ ID NO:389. In some embodiments, the crRNA sequence has at least 95% identity to SEQ ID NO:389. In some embodiments, the crRNA sequence is SEQ ID NO:389.

In some embodiments, the at least one guide RNA targets a canine IL-1α gene, and includes a crRNA sequence having at least 75% identity to a sequence selected from the group consisting of SEQ ID NOs: 202-216, 552-590, and 741-770. In some embodiments, the crRNA sequence has at least 80% identity to a sequence selected from the group consisting of SEQ ID NOs: 202-216, 552-590, and 741-770. In some embodiments, the crRNA sequence has at least 85% identity to a sequence selected from the group consisting of SEQ ID NOs: 202-216, 552-590, and 741-770. In some embodiments, the crRNA sequence has at least 90% identity to a sequence selected from the group consisting of SEQ ID NOs: 202-216, 552-590, and 741-770. In some embodiments, the crRNA sequence has at least 95% identity to a sequence selected from the group consisting of SEQ ID NOs: 202-216, 552-590, and 741-770. In some embodiments, the crRNA sequence is selected from the group consisting of SEQ ID NOs: 202-216, 552-590, and 741-770.

In some embodiments, the at least one guide RNA targets a canine IL-1α gene, and includes a crRNA sequence having at least 75% identity to SEQ ID NO:552. In some embodiments, the crRNA sequence has at least 80% identity to SEQ ID NO:552. In some embodiments, the crRNA sequence has at least 85% identity to SEQ ID NO:552. In some embodiments, the crRNA sequence has at least 90% identity to SEQ ID NO:552. In some embodiments, the crRNA sequence has at least 95% identity to SEQ ID NO:552. In some embodiments, the crRNA sequence is SEQ ID NO:552.

In some embodiments, the at least one guide RNA targets a canine IL-1α gene, and includes a crRNA sequence having at least 75% identity to SEQ ID NO:554. In some embodiments, the crRNA sequence has at least 80% identity to SEQ ID NO:554. In some embodiments, the crRNA sequence has at least 85% identity to SEQ ID NO:554. In some embodiments, the crRNA sequence has at least 90% identity to SEQ ID NO:554. In some embodiments, the crRNA sequence has at least 95% identity to SEQ ID NO:554. In some embodiments, the crRNA sequence is SEQ ID NO:554.

In some embodiments, the at least one guide RNA targets a canine IL-1α gene, and includes a crRNA sequence having at least 75% identity to SEQ ID NO:578. In some embodiments, the crRNA sequence has at least 80% identity to SEQ ID NO:578. In some embodiments, the crRNA sequence has at least 85% identity to SEQ ID NO:578. In some embodiments, the crRNA sequence has at least 90% identity to SEQ ID NO:578. In some embodiments, the crRNA sequence has at least 95% identity to SEQ ID NO:578. In some embodiments, the crRNA sequence is SEQ ID NO:578.

In some embodiments, the at least one guide RNA targets a canine IL-1α gene, and includes a crRNA sequence having at least 75% identity to SEQ ID NO:579. In some embodiments, the crRNA sequence has at least 80% identity to SEQ ID NO:579. In some embodiments, the crRNA sequence has at least 85% identity to SEQ ID NO:579. In some embodiments, the crRNA sequence has at least 90% identity to SEQ ID NO:579. In some embodiments, the crRNA sequence has at least 95% identity to SEQ ID NO:579. In some embodiments, the crRNA sequence is SEQ ID NO:579.

In some embodiments, the at least one guide RNA targets a canine IL-1α gene, and includes a crRNA sequence that is complementary to a target sequence in exon 2 of the IL-1α gene. In some embodiments, the crRNA sequence forms no more than 5 mismatches with the target sequence in exon 2 of the canine IL-1α gene. In some embodiments, the crRNA sequence forms no more than 4 mismatches with the target sequence in exon 2 of the canine IL-1α gene. In some embodiments, the crRNA sequence forms no more than 3 mismatches with the target sequence in exon 2 of the canine IL-1α gene. In some embodiments, the crRNA sequence forms no more than 2 mismatches with the target sequence in exon 2 of the canine IL-1α gene. In some embodiments, the crRNA sequence forms no more than 1 mismatch with the target sequence in exon 2 of the canine IL-1α gene. In some embodiments, the crRNA sequence forms no mismatches with the target sequence in exon 2 of the canine IL-1α gene.

In some embodiments, the at least one guide RNA targets a canine IL-1α gene, and includes a crRNA sequence that is complementary to a target sequence in exon 3 of the IL-1α gene. In some embodiments, the crRNA sequence forms no more than 5 mismatches with the target sequence in exon 3 of the canine IL-1α gene. In some embodiments, the crRNA sequence forms no more than 4 mismatches with the target sequence in exon 3 of the canine IL-1α gene. In some embodiments, the crRNA sequence forms no more than 3 mismatches with the target sequence in exon 3 of the canine IL-1α gene. In some embodiments, the crRNA sequence forms no more than 2 mismatches with the target sequence in exon 3 of the canine IL-1α gene. In some embodiments, the crRNA sequence forms no more than 1 mismatch with the target sequence in exon 3 of the canine IL-1α gene. In some embodiments, the crRNA sequence forms no mismatches with the target sequence in exon 3 of the canine IL-1α gene.

In some embodiments, the at least one guide RNA targets a canine IL-1α gene, and includes a crRNA sequence that is complementary to a target sequence in exon 4 of the IL-1α gene. In some embodiments, the crRNA sequence forms no more than 5 mismatches with the target sequence in exon 4 of the canine IL-1α gene. In some embodiments, the crRNA sequence forms no more than 4 mismatches with the target sequence in exon 4 of the canine IL-1α gene. In some embodiments, the crRNA sequence forms no more than 3 mismatches with the target sequence in exon 4 of the canine IL-1α gene. In some embodiments, the crRNA sequence forms no more than 2 mismatches with the target sequence in exon 4 of the canine IL-1α gene. In some embodiments, the crRNA sequence forms no more than 1 mismatch with the target sequence in exon 4 of the canine IL-1α gene. In some embodiments, the crRNA sequence forms no mismatches with the target sequence in exon 4 of the canine IL-1α gene.

In some embodiments, the at least one guide RNA targets a canine IL-1α gene, and includes a crRNA sequence that is complementary to a target sequence in exon 5 of the IL-1α gene. In some embodiments, the crRNA sequence forms no more than 5 mismatches with the target sequence in exon 5 of the canine IL-1α gene. In some embodiments, the crRNA sequence forms no more than 4 mismatches with the target sequence in exon 5 of the canine IL-1α gene. In some embodiments, the crRNA sequence forms no more than 3 mismatches with the target sequence in exon 5 of the canine IL-1α gene. In some embodiments, the crRNA sequence forms no more than 2 mismatches with the target sequence in exon 5 of the canine IL-1α gene. In some embodiments, the crRNA sequence forms no more than 1 mismatch with the target sequence in exon 5 of the canine IL-1α gene. In some embodiments, the crRNA sequence forms no mismatches with the target sequence in exon 5 of the canine IL-1α gene.

In some embodiments, the at least one guide RNA targets a canine IL-1α gene, and includes a crRNA sequence that is complementary to a target sequence in exon 6 of the IL-1α gene. In some embodiments, the crRNA sequence forms no more than 5 mismatches with the target sequence in exon 6 of the canine IL-1α gene. In some embodiments, the crRNA sequence forms no more than 4 mismatches with the target sequence in exon 6 of the canine IL-1α gene. In some embodiments, the crRNA sequence forms no more than 3 mismatches with the target sequence in exon 6 of the canine IL-1α gene. In some embodiments, the crRNA sequence forms no more than 2 mismatches with the target sequence in exon 6 of the canine IL-1α gene. In some embodiments, the crRNA sequence forms no more than 1 mismatch with the target sequence in exon 6 of the canine IL-1α gene. In some embodiments, the crRNA sequence forms no mismatches with the target sequence in exon 6 of the canine IL-1α gene.

In some embodiments, the at least one guide RNA targets a canine IL-1α gene, and includes a crRNA sequence that is complementary to a target sequence in exon 7 of the IL-1α gene. In some embodiments, the crRNA sequence forms no more than 5 mismatches with the target sequence in exon 7 of the canine IL-1α gene. In some embodiments, the crRNA sequence forms no more than 4 mismatches with the target sequence in exon 7 of the canine IL-1α gene. In some embodiments, the crRNA sequence forms no more than 3 mismatches with the target sequence in exon 7 of the canine IL-1α gene. In some embodiments, the crRNA sequence forms no more than 2 mismatches with the target sequence in exon 7 of the canine IL-1α gene. In some embodiments, the crRNA sequence forms no more than 1 mismatch with the target sequence in exon 7 of the canine IL-1α gene. In some embodiments, the crRNA sequence forms no mismatches with the target sequence in exon 7 of the canine IL-1α gene.

In some embodiments, the at least one guide RNA targets a canine IL-1α gene, and includes a crRNA sequence that is complementary to a target sequence in exon 2 or exon 3 of the IL-1α gene. In some embodiments, the at least one guide RNA targets a canine IL-1α gene, and includes a crRNA sequence that is complementary to a target sequence in exon 2 or exon 4 of the IL-1α gene. In some embodiments, the at least one guide RNA targets a canine IL-1α gene, and includes a crRNA sequence that is complementary to a target sequence in exon 2 or exon 5 of the IL-1α gene. In some embodiments, the at least one guide RNA targets a canine IL-1α gene, and includes a crRNA sequence that is complementary to a target sequence in exon 2 or exon 6 of the IL-1α gene. In some embodiments, the at least one guide RNA targets a canine IL-1α gene, and includes a crRNA sequence that is complementary to a target sequence in exon 2 or exon 7 of the IL-1α gene.

In some embodiments, the at least one guide RNA targets a canine IL-1α gene, and includes a crRNA sequence that is complementary to a target sequence in exon 3 or exon 4 of the IL-1α gene. In some embodiments, the at least one guide RNA targets a canine IL-1α gene, and includes a crRNA sequence that is complementary to a target sequence in exon 3 or exon 5 of the IL-1α gene. In some embodiments, the at least one guide RNA targets a canine IL-1α gene, and includes a crRNA sequence that is complementary to a target sequence in exon 3 or exon 6 of the IL-1α gene. In some embodiments, the at least one guide RNA targets a canine IL-1α gene, and includes a crRNA sequence that is complementary to a target sequence in exon 3 or exon 7 of the IL-1α gene.

In some embodiments, the at least one guide RNA targets a canine IL-1α gene, and includes a crRNA sequence that is complementary to a target sequence in exon 4 or exon 5 of the IL-1α gene. In some embodiments, the at least one guide RNA targets a canine IL-1α gene, and includes a crRNA sequence that is complementary to a target sequence in exon 4 or exon 6 of the IL-1α gene. In some embodiments, the at least one guide RNA targets a canine IL-1α gene, and includes a crRNA sequence that is complementary to a target sequence in exon 4 or exon 7 of the IL-1α gene.

In some embodiments, the at least one guide RNA targets a canine IL-1α gene, and includes a crRNA sequence that is complementary to a target sequence in exon 5 or exon 6 of the IL-1α gene. In some embodiments, the at least one guide RNA targets a canine IL-1α gene, and includes a crRNA sequence that is complementary to a target sequence in exon 5 or exon 7 of the IL-1α gene. In some embodiments, the at least one guide RNA targets a canine IL-1α gene, and includes a crRNA sequence that is complementary to a target sequence in exon 6 or exon 7 of the IL-1α gene.

In some embodiments, the at least one guide RNA targets a canine IL-1α gene, and includes a crRNA sequence that is complementary to a target sequence in exon 2, exon 3, or exon 4 of the IL-1α gene. In some embodiments, the at least one guide RNA targets a canine IL-1α gene, and includes a crRNA sequence that is complementary to a target sequence in exon 2, exon 3, or exon 5 of the IL-1α gene. In some embodiments, the at least one guide RNA targets a canine IL-1α gene, and includes a crRNA sequence that is complementary to a target sequence in exon 2, exon 3, or exon 6 of the IL-1α gene. In some embodiments, the at least one guide RNA targets a canine IL-1α gene, and includes a crRNA sequence that is complementary to a target sequence in exon 2, exon 3, or exon 7 of the IL-1α gene. In some embodiments, the at least one guide RNA targets a canine IL-1α gene, and includes a crRNA sequence that is complementary to a target sequence in exon 2, exon 4, or exon 5 of the IL-1α gene. In some embodiments, the at least one guide RNA targets a canine IL-1α gene, and includes a crRNA sequence that is complementary to a target sequence in exon 2, exon 4, or exon 6 of the IL-1α gene. In some embodiments, the at least one guide RNA targets a canine IL-1α gene, and includes a crRNA sequence that is complementary to a target sequence in exon 2, exon 4, or exon 7 of the IL-1α gene. In some embodiments, the at least one guide RNA targets a canine IL-1α gene, and includes a crRNA sequence that is complementary to a target sequence in exon 2, exon 5, or exon 6 of the IL-1α gene. In some embodiments, the at least one guide RNA targets a canine IL-1α gene, and includes a crRNA sequence that is complementary to a target sequence in exon 2, exon 5, or exon 7 of the IL-1α gene. In some embodiments, the at least one guide RNA targets a canine IL-1α gene, and includes a crRNA sequence that is complementary to a target sequence in exon 2, exon 6, or exon 7 of the IL-1α gene.

In some embodiments, the at least one guide RNA targets a canine IL-1α gene, and includes a crRNA sequence that is complementary to a target sequence in exon 3, exon 4, or exon 5 of the IL-1α gene. In some embodiments, the at least one guide RNA targets a canine IL-1α gene, and includes a crRNA sequence that is complementary to a target sequence in exon 3, exon 4, or exon 6 of the IL-1α gene. In some embodiments, the at least one guide RNA targets a canine IL-1α gene, and includes a crRNA sequence that is complementary to a target sequence in exon 3, exon 4, or exon 7 of the IL-1α gene. In some embodiments, the at least one guide RNA targets a canine IL-1α gene, and includes a crRNA sequence that is complementary to a target sequence in exon 3, exon 5, or exon 6 of the IL-1α gene. In some embodiments, the at least one guide RNA targets a canine IL-1α gene, and includes a crRNA sequence that is complementary to a target sequence in exon 3, exon 5, or exon 7 of the IL-1α gene. In some embodiments, the at least one guide RNA targets a canine IL-1α gene, and includes a crRNA sequence that is complementary to a target sequence in exon 3, exon 6, or exon 7 of the IL-1α gene.

In some embodiments, the at least one guide RNA targets a canine IL-1α gene, and includes a crRNA sequence that is complementary to a target sequence in exon 4, exon 5, or exon 6 of the IL-1α gene. In some embodiments, the at least one guide RNA targets a canine IL-1α gene, and includes a crRNA sequence that is complementary to a target sequence in exon 4, exon 5, or exon 7 of the IL-1α gene. In some embodiments, the at least one guide RNA targets a canine IL-1α gene, and includes a crRNA sequence that is complementary to a target sequence in exon 4, exon 6, or exon 7 of the IL-1α gene. In some embodiments, the at least one guide RNA targets a canine IL-1α gene, and includes a crRNA sequence that is complementary to a target sequence in exon 5, exon 6, or exon 7 of the IL-1α gene.

In some embodiments, the at least one guide RNA targets a canine IL-1α gene, and includes a crRNA sequence that is complementary to a target sequence in exon 2, exon 3, exon 4, or exon 5 of the IL-1α gene. In some embodiments, the at least one guide RNA targets a canine IL-1α gene, and includes a crRNA sequence that is complementary to a target sequence in exon 2, exon 3, exon 4, or exon 6 of the IL-1α gene. In some embodiments, the at least one guide RNA targets a canine IL-1α gene, and includes a crRNA sequence that is complementary to a target sequence in exon 2, exon 3, exon 4, or exon 7 of the IL-1α gene. In some embodiments, the at least one guide RNA targets a canine IL-1α gene, and includes a crRNA sequence that is complementary to a target sequence in exon 2, exon 4, exon 5, or exon 6 of the IL-1α gene. In some embodiments, the at least one guide RNA targets a canine IL-1α gene, and includes a crRNA sequence that is complementary to a target sequence in exon 2, exon 4, exon 5, or exon 7 of the IL-1α gene. In some embodiments, the at least one guide RNA targets a canine IL-1α gene, and includes a crRNA sequence that is complementary to a target sequence in exon 2, exon 4, exon 6, or exon 7 of the IL-1α gene. In some embodiments, the at least one guide RNA targets a canine IL-1α gene, and includes a crRNA sequence that is complementary to a target sequence in exon 2, exon 5, exon 6, or exon 7 of the IL-1α gene.

In some embodiments, the at least one guide RNA targets a canine IL-1α gene, and includes a crRNA sequence that is complementary to a target sequence in exon 3, exon 4, exon 5, or exon 6 of the IL-1α gene. In some embodiments, the at least one guide RNA targets a canine IL-1α gene, and includes a crRNA sequence that is complementary to a target sequence in exon 3, exon 4, exon 5, or exon 7 of the IL-1α gene. In some embodiments, the at least one guide RNA targets a canine IL-1α gene, and includes a crRNA sequence that is complementary to a target sequence in exon 3, exon 4, exon 6, or exon 7 of the IL-1α gene. In some embodiments, the at least one guide RNA targets a canine IL-1α gene, and includes a crRNA sequence that is complementary to a target sequence in exon 3, exon 5, exon 6, or exon 7 of the IL-1α gene. In some embodiments, the at least one guide RNA targets a canine IL-1α gene, and includes a crRNA sequence that is complementary to a target sequence in exon 4, exon 5, exon 6, or exon 7 of the IL-1α gene.

In some embodiments, the at least one guide RNA targets a canine IL-1α gene, and includes a crRNA sequence that is complementary to a target sequence in exon 2, exon 3, exon 4, exon 5, or exon 6 of the IL-1α gene. In some embodiments, the at least one guide RNA targets a canine IL-1α gene, and includes a crRNA sequence that is complementary to a target sequence in exon 2, exon 3, exon 4, exon 5, or exon 7 of the IL-1α gene. In some embodiments, the at least one guide RNA targets a canine IL-1α gene, and includes a crRNA sequence that is complementary to a target sequence in exon 3, exon 4, exon 5, exon 6, or exon 7 of the IL-1α gene. In some embodiments, the at least one guide RNA targets a canine IL-1α gene, and includes a crRNA sequence that is complementary to a target sequence in exon 2, exon 4, exon 5, exon 6, or exon 7 of the IL-1α gene. In some embodiments, the at least one guide RNA targets a canine IL-1α gene, and includes a crRNA sequence that is complementary to a target sequence in exon 2, exon 3, exon 5, exon 6, or exon 6 of the IL-1α gene. In some embodiments, the at least one guide RNA targets a canine IL-1α gene, and includes a crRNA sequence that is complementary to a target sequence in exon 2, exon 3, exon 4, exon 6, or exon 7 of the IL-1α gene. In some embodiments, the at least one guide RNA targets a canine IL-1α gene, and includes a crRNA sequence that is complementary to a target sequence in exon 2, exon 3, exon 4, exon 5, exon 6, or exon 7 of the IL-1α gene.

In some embodiments, the at least one guide RNA targets a canine IL-1β gene, and includes a crRNA sequence having at least 75% identity to a sequence selected from the group consisting of SEQ ID NOs: 217-235, 497-551, and 771-800. In some embodiments, the crRNA sequence has at least 80% identity to a sequence selected from the group consisting of SEQ ID NOs: 217-235, 497-551, and 771-800. In some embodiments, the crRNA sequence has at least 85% identity to a sequence selected from the group consisting of SEQ ID NOs: 217-235, 497-551, and 771-800. In some embodiments, the crRNA sequence has at least 90% identity to a sequence selected from the group consisting of SEQ ID NOs: 217-235, 497-551, and 771-800. In some embodiments, the crRNA sequence has at least 95% identity to a sequence selected from the group consisting of SEQ ID NOs: 217-235, 497-551, and 771-800. In some embodiments, the crRNA sequence is selected from the group consisting of SEQ ID NOs: 217-235, 497-551, and 771-800.

In some embodiments, the at least one guide RNA targets a canine IL-1β gene, and includes a crRNA sequence having at least 75% identity to SEQ ID NO:498. In some embodiments, the crRNA sequence has at least 80% identity to SEQ ID NO:498. In some embodiments, the crRNA sequence has at least 85% identity to SEQ ID NO:498. In some embodiments, the crRNA sequence has at least 90% identity to SEQ ID NO:498. In some embodiments, the crRNA sequence has at least 95% identity to SEQ ID NO:498. In some embodiments, the crRNA sequence is SEQ ID NO:498.

In some embodiments, the at least one guide RNA targets a canine IL-1β gene, and includes a crRNA sequence having at least 75% identity to SEQ ID NO:506. In some embodiments, the crRNA sequence has at least 80% identity to SEQ ID NO:506. In some embodiments, the crRNA sequence has at least 85% identity to SEQ ID NO:506. In some embodiments, the crRNA sequence has at least 90% identity to SEQ ID NO:506. In some embodiments, the crRNA sequence has at least 95% identity to SEQ ID NO:506. In some embodiments, the crRNA sequence is SEQ ID NO:506.

In some embodiments, the at least one guide RNA targets a canine IL-1β gene, and includes a crRNA sequence that is complementary to a target sequence in exon 2 of the IL-1β gene. In some embodiments, the crRNA sequence forms no more than 5 mismatches with the target sequence in exon 2 of the canine IL-1β gene. In some embodiments, the crRNA sequence forms no more than 4 mismatches with the target sequence in exon 2 of the canine IL-1β gene. In some embodiments, the crRNA sequence forms no more than 3 mismatches with the target sequence in exon 2 of the canine IL-1β gene. In some embodiments, the crRNA sequence forms no more than 2 mismatches with the target sequence in exon 2 of the canine IL-1β gene. In some embodiments, the crRNA sequence forms no more than 1 mismatch with the target sequence in exon 2 of the canine IL-1β gene. In some embodiments, the crRNA sequence forms no mismatches with the target sequence in exon 2 of the canine IL-1β gene.

In some embodiments, the at least one guide RNA targets a canine IL-1β gene, and includes a crRNA sequence that is complementary to a target sequence in exon 3 of the IL-1β gene. In some embodiments, the crRNA sequence forms no more than 5 mismatches with the target sequence in exon 3 of the canine IL-1β gene. In some embodiments, the crRNA sequence forms no more than 4 mismatches with the target sequence in exon 3 of the canine IL-1β gene. In some embodiments, the crRNA sequence forms no more than 3 mismatches with the target sequence in exon 3 of the canine IL-1β gene. In some embodiments, the crRNA sequence forms no more than 2 mismatches with the target sequence in exon 3 of the canine IL-1β gene. In some embodiments, the crRNA sequence forms no more than 1 mismatch with the target sequence in exon 3 of the canine IL-1β gene. In some embodiments, the crRNA sequence forms no mismatches with the target sequence in exon 3 of the canine IL-1β gene.

In some embodiments, the at least one guide RNA targets a canine IL-1β gene, and includes a crRNA sequence that is complementary to a target sequence in exon 4 of the IL-1β gene. In some embodiments, the crRNA sequence forms no more than 5 mismatches with the target sequence in exon 4 of the canine IL-1β gene. In some embodiments, the crRNA sequence forms no more than 4 mismatches with the target sequence in exon 4 of the canine IL-1β gene. In some embodiments, the crRNA sequence forms no more than 3 mismatches with the target sequence in exon 4 of the canine IL-1β gene. In some embodiments, the crRNA sequence forms no more than 2 mismatches with the target sequence in exon 4 of the canine IL-1β gene. In some embodiments, the crRNA sequence forms no more than 1 mismatch with the target sequence in exon 4 of the canine IL-1β gene. In some embodiments, the crRNA sequence forms no mismatches with the target sequence in exon 4 of the canine IL-1β gene.

In some embodiments, the at least one guide RNA targets a canine IL-1β gene, and includes a crRNA sequence that is complementary to a target sequence in exon 5 of the IL-1β gene. In some embodiments, the crRNA sequence forms no more than 5 mismatches with the target sequence in exon 5 of the canine IL-1β gene. In some embodiments, the crRNA sequence forms no more than 4 mismatches with the target sequence in exon 5 of the canine IL-1β gene. In some embodiments, the crRNA sequence forms no more than 3 mismatches with the target sequence in exon 5 of the canine IL-1β gene. In some embodiments, the crRNA sequence forms no more than 2 mismatches with the target sequence in exon 5 of the canine IL-1β gene. In some embodiments, the crRNA sequence forms no more than 1 mismatch with the target sequence in exon 5 of the canine IL-1β gene. In some embodiments, the crRNA sequence forms no mismatches with the target sequence in exon 5 of the canine IL-1β gene.

In some embodiments, the at least one guide RNA targets a canine IL-1β gene, and includes a crRNA sequence that is complementary to a target sequence in exon 6 of the IL-1β gene. In some embodiments, the crRNA sequence forms no more than 5 mismatches with the target sequence in exon 6 of the canine IL-1β gene. In some embodiments, the crRNA sequence forms no more than 4 mismatches with the target sequence in exon 6 of the canine IL-1β gene. In some embodiments, the crRNA sequence forms no more than 3 mismatches with the target sequence in exon 6 of the canine IL-1β gene. In some embodiments, the crRNA sequence forms no more than 2 mismatches with the target sequence in exon 6 of the canine IL-1β gene. In some embodiments, the crRNA sequence forms no more than 1 mismatch with the target sequence in exon 6 of the canine IL-1β gene. In some embodiments, the crRNA sequence forms no mismatches with the target sequence in exon 6 of the canine IL-1β gene.

In some embodiments, the at least one guide RNA targets a canine IL-1β gene, and includes a crRNA sequence that is complementary to a target sequence in exon 7 of the IL-1β gene. In some embodiments, the crRNA sequence forms no more than 5 mismatches with the target sequence in exon 7 of the canine IL-1β gene. In some embodiments, the crRNA sequence forms no more than 4 mismatches with the target sequence in exon 7 of the canine IL-1β gene. In some embodiments, the crRNA sequence forms no more than 3 mismatches with the target sequence in exon 7 of the canine IL-1β gene. In some embodiments, the crRNA sequence forms no more than 2 mismatches with the target sequence in exon 7 of the canine IL-1β gene. In some embodiments, the crRNA sequence forms no more than 1 mismatch with the target sequence in exon 7 of the canine IL-1β gene. In some embodiments, the crRNA sequence forms no mismatches with the target sequence in exon 7 of the canine IL-1β gene.

In some embodiments, the at least one guide RNA targets a canine IL-1β gene, and includes a crRNA sequence that is complementary to a target sequence in exon 2 or exon 3 of the IL-1β gene. In some embodiments, the at least one guide RNA targets a canine IL-1β gene, and includes a crRNA sequence that is complementary to a target sequence in exon 2 or exon 4 of the IL-1β gene. In some embodiments, the at least one guide RNA targets a canine IL-1β gene, and includes a crRNA sequence that is complementary to a target sequence in exon 2 or exon 5 of the IL-1β gene. In some embodiments, the at least one guide RNA targets a canine IL-1β gene, and includes a crRNA sequence that is complementary to a target sequence in exon 2 or exon 6 of the IL-1β gene. In some embodiments, the at least one guide RNA targets a canine IL-1β gene, and includes a crRNA sequence that is complementary to a target sequence in exon 2 or exon 7 of the IL-1β gene.

In some embodiments, the at least one guide RNA targets a canine IL-1β gene, and includes a crRNA sequence that is complementary to a target sequence in exon 3 or exon 4 of the IL-1β gene. In some embodiments, the at least one guide RNA targets a canine IL-1β gene, and includes a crRNA sequence that is complementary to a target sequence in exon 3 or exon 5 of the IL-1β gene. In some embodiments, the at least one guide RNA targets a canine IL-1β gene, and includes a crRNA sequence that is complementary to a target sequence in exon 3 or exon 6 of the IL-1β gene. In some embodiments, the at least one guide RNA targets a canine IL-1β gene, and includes a crRNA sequence that is complementary to a target sequence in exon 3 or exon 7 of the IL-1β gene.

In some embodiments, the at least one guide RNA targets a canine IL-1β gene, and includes a crRNA sequence that is complementary to a target sequence in exon 4 or exon 5 of the IL-1β gene. In some embodiments, the at least one guide RNA targets a canine IL-1β gene, and includes a crRNA sequence that is complementary to a target sequence in exon 4 or exon 6 of the IL-1β gene. In some embodiments, the at least one guide RNA targets a canine IL-1β gene, and includes a crRNA sequence that is complementary to a target sequence in exon 4 or exon 7 of the IL-1β gene.

In some embodiments, the at least one guide RNA targets a canine IL-1β gene, and includes a crRNA sequence that is complementary to a target sequence in exon 5 or exon 6 of the IL-1β gene. In some embodiments, the at least one guide RNA targets a canine IL-1β gene, and includes a crRNA sequence that is complementary to a target sequence in exon 5 or exon 7 of the IL-1β gene. In some embodiments, the at least one guide RNA targets a canine IL-1β gene, and includes a crRNA sequence that is complementary to a target sequence in exon 6 or exon 7 of the IL-1β gene.

In some embodiments, the at least one guide RNA targets a canine IL-1β gene, and includes a crRNA sequence that is complementary to a target sequence in exon 2, exon 3, or exon 4 of the IL-1β gene. In some embodiments, the at least one guide RNA targets a canine IL-1β gene, and includes a crRNA sequence that is complementary to a target sequence in exon 2, exon 3, or exon 5 of the IL-1β gene. In some embodiments, the at least one guide RNA targets a canine IL-1β gene, and includes a crRNA sequence that is complementary to a target sequence in exon 2, exon 3, or exon 6 of the IL-1β gene. In some embodiments, the at least one guide RNA targets a canine IL-1β gene, and includes a crRNA sequence that is complementary to a target sequence in exon 2, exon 3, or exon 7 of the IL-1 gene. In some embodiments, the at least one guide RNA targets a canine IL-1β gene, and includes a crRNA sequence that is complementary to a target sequence in exon 2, exon 4, or exon 5 of the IL-1β gene. In some embodiments, the at least one guide RNA targets a canine IL-1β gene, and includes a crRNA sequence that is complementary to a target sequence in exon 2, exon 4, or exon 6 of the IL-1β gene. In some embodiments, the at least one guide RNA targets a canine IL-1β gene, and includes a crRNA sequence that is complementary to a target sequence in exon 2, exon 4, or exon 7 of the IL-1β gene. In some embodiments, the at least one guide RNA targets a canine IL-1β gene, and includes a crRNA sequence that is complementary to a target sequence in exon 2, exon 5, or exon 6 of the IL-1β gene. In some embodiments, the at least one guide RNA targets a canine IL-1β gene, and includes a crRNA sequence that is complementary to a target sequence in exon 2, exon 5, or exon 7 of the IL-1β gene. In some embodiments, the at least one guide RNA targets a canine IL-1β gene, and includes a crRNA sequence that is complementary to a target sequence in exon 2, exon 6, or exon 7 of the IL-1β gene.

In some embodiments, the at least one guide RNA targets a canine IL-1β gene, and includes a crRNA sequence that is complementary to a target sequence in exon 3, exon 4, or exon 5 of the IL-1β gene. In some embodiments, the at least one guide RNA targets a canine IL-1β gene, and includes a crRNA sequence that is complementary to a target sequence in exon 3, exon 4, or exon 6 of the IL-1β gene. In some embodiments, the at least one guide RNA targets a canine IL-1β gene, and includes a crRNA sequence that is complementary to a target sequence in exon 3, exon 4, or exon 7 of the IL-1β gene. In some embodiments, the at least one guide RNA targets a canine IL-1β gene, and includes a crRNA sequence that is complementary to a target sequence in exon 3, exon 5, or exon 6 of the IL-1β gene. In some embodiments, the at least one guide RNA targets a canine IL-1β gene, and includes a crRNA sequence that is complementary to a target sequence in exon 3, exon 5, or exon 7 of the IL-1β gene. In some embodiments, the at least one guide RNA targets a canine IL-1β gene, and includes a crRNA sequence that is complementary to a target sequence in exon 3, exon 6, or exon 7 of the IL-1β gene.

In some embodiments, the at least one guide RNA targets a canine IL-1β gene, and includes a crRNA sequence that is complementary to a target sequence in exon 4, exon 5, or exon 6 of the IL-1β gene. In some embodiments, the at least one guide RNA targets a canine IL-1β gene, and includes a crRNA sequence that is complementary to a target sequence in exon 4, exon 5, or exon 7 of the IL-1β gene. In some embodiments, the at least one guide RNA targets a canine IL-1β gene, and includes a crRNA sequence that is complementary to a target sequence in exon 4, exon 6, or exon 7 of the IL-1β gene. In some embodiments, the at least one guide RNA targets a canine IL-1β gene, and includes a crRNA sequence that is complementary to a target sequence in exon 5, exon 6, or exon 7 of the IL-1β gene.

In some embodiments, the at least one guide RNA targets a canine IL-1β gene, and includes a crRNA sequence that is complementary to a target sequence in exon 2, exon 3, exon 4, or exon 5 of the IL-1β gene. In some embodiments, the at least one guide RNA targets a canine IL-1β gene, and includes a crRNA sequence that is complementary to a target sequence in exon 2, exon 3, exon 4, or exon 6 of the IL-1β gene. In some embodiments, the at least one guide RNA targets a canine IL-1β gene, and includes a crRNA sequence that is complementary to a target sequence in exon 2, exon 3, exon 4, or exon 7 of the IL-1 gene. In some embodiments, the at least one guide RNA targets a canine IL-1β gene, and includes a crRNA sequence that is complementary to a target sequence in exon 2, exon 4, exon 5, or exon 6 of the IL-1β gene. In some embodiments, the at least one guide RNA targets a canine IL-1β gene, and includes a crRNA sequence that is complementary to a target sequence in exon 2, exon 4, exon 5, or exon 7 of the IL-1β gene. In some embodiments, the at least one guide RNA targets a canine IL-1β gene, and includes a crRNA sequence that is complementary to a target sequence in exon 2, exon 4, exon 6, or exon 7 of the IL-1β gene. In some embodiments, the at least one guide RNA targets a canine IL-1β gene, and includes a crRNA sequence that is complementary to a target sequence in exon 2, exon 5, exon 6, or exon 7 of the IL-1β gene.

In some embodiments, the at least one guide RNA targets a canine IL-1β gene, and includes a crRNA sequence that is complementary to a target sequence in exon 3, exon 4, exon 5, or exon 6 of the IL-1β gene. In some embodiments, the at least one guide RNA targets a canine IL-1β gene, and includes a crRNA sequence that is complementary to a target sequence in exon 3, exon 4, exon 5, or exon 7 of the IL-1β gene. In some embodiments, the at least one guide RNA targets a canine IL-1β gene, and includes a crRNA sequence that is complementary to a target sequence in exon 3, exon 4, exon 6, or exon 7 of the IL-1β gene. In some embodiments, the at least one guide RNA targets a canine IL-1β gene, and includes a crRNA sequence that is complementary to a target sequence in exon 3, exon 5, exon 6, or exon 7 of the IL-1β gene. In some embodiments, the at least one guide RNA targets a canine IL-1β gene, and includes a crRNA sequence that is complementary to a target sequence in exon 4, exon 5, exon 6, or exon 7 of the IL-1β gene.

In some embodiments, the at least one guide RNA targets a canine IL-1β gene, and includes a crRNA sequence that is complementary to a target sequence in exon 2, exon 3, exon 4, exon 5, or exon 6 of the IL-1β gene. In some embodiments, the at least one guide RNA targets a canine IL-1β gene, and includes a crRNA sequence that is complementary to a target sequence in exon 2, exon 3, exon 4, exon 5, or exon 7 of the IL-1β gene. In some embodiments, the at least one guide RNA targets a canine IL-1β gene, and includes a crRNA sequence that is complementary to a target sequence in exon 3, exon 4, exon 5, exon 6, or exon 7 of the IL-1β gene. In some embodiments, the at least one guide RNA targets a canine IL-1β gene, and includes a crRNA sequence that is complementary to a target sequence in exon 2, exon 4, exon 5, exon 6, or exon 7 of the IL-1β gene. In some embodiments, the at least one guide RNA targets a canine IL-1β gene, and includes a crRNA sequence that is complementary to a target sequence in exon 2, exon 3, exon 5, exon 6, or exon 6 of the IL-1β gene. In some embodiments, the at least one guide RNA targets a canine IL-1β gene, and includes a crRNA sequence that is complementary to a target sequence in exon 2, exon 3, exon 4, exon 6, or exon 7 of the IL-1β gene. In some embodiments, the at least one guide RNA targets a canine IL-1β gene, and includes a crRNA sequence that is complementary to a target sequence in exon 2, exon 3, exon 4, exon 5, exon 6, or exon 7 of the IL-1β gene.

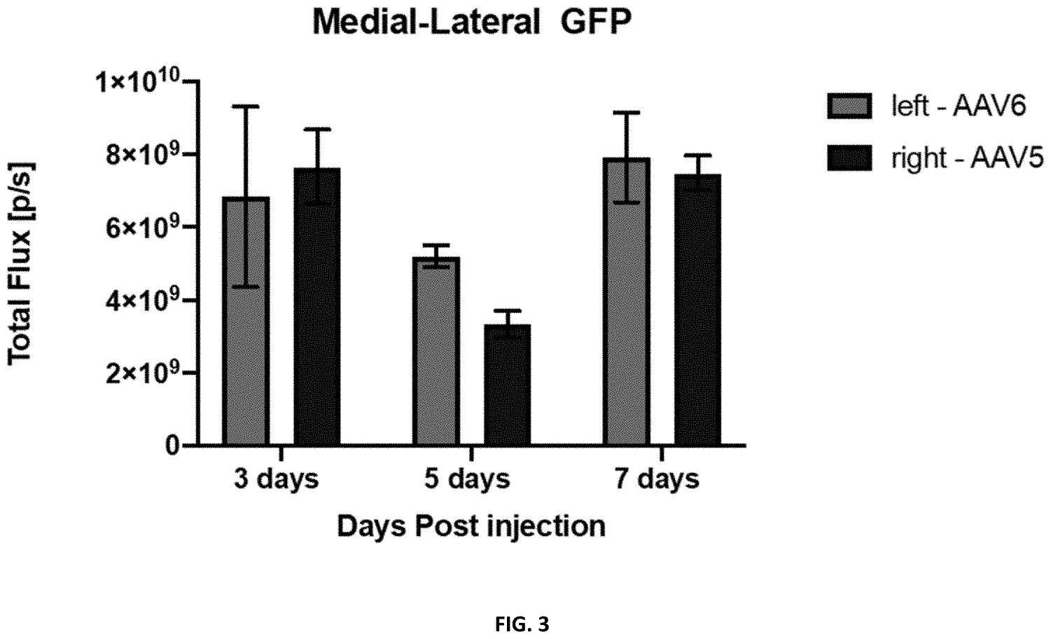

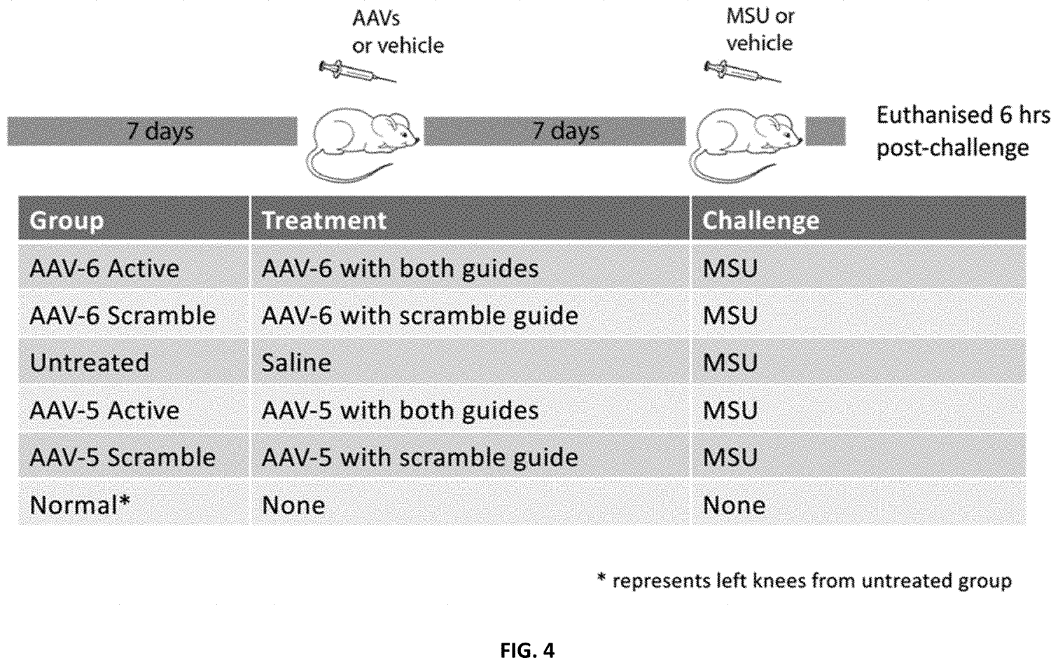

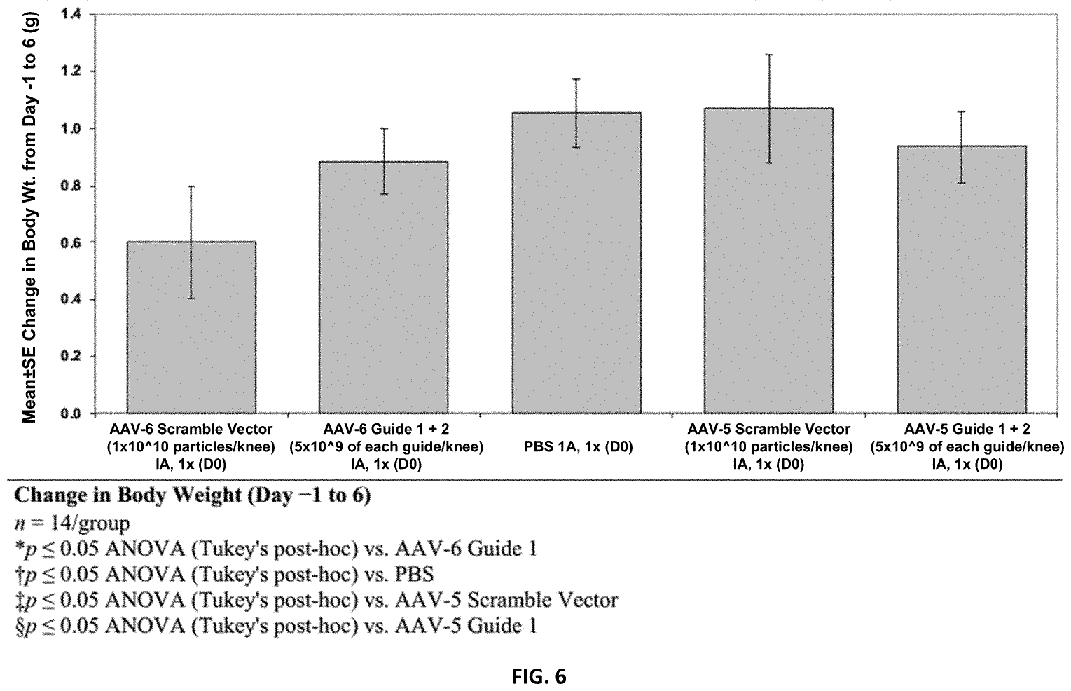

Generally, the crRNA sequences described herein may include one or more nucleotide substitutions, e.g., relative to the reverse complement of the target sequence. Guidance for making nucleotide substitutions can be found, for example, in Jiang et al. and Doudna (Jiang and Doudna, Annu. Rev. Biophys., 46:505-29 (2017)), the content of which is incorporated herein by reference, in its entirety, for all purposes. Specifically, Jiang and Doudna considers molecular structures generated for many different confirmations of the CRISPR/Cas9 system, ranging from apo Cas9 protein (FIG. 3) to Cas9-sgRNA complexes bound to the target strand of an invaded double-stranded DNA molecule (FIGS. 5 and 7), to arrive at a detailed molecule model of CRISPR/Cas9 binding and cleavage in FIG. 6. From these molecular models, the person of ordinary skill in the art would know which nucleotide positions in the crRNA sequence would be more tolerant of mismatches with the target sequence.

For instance, Jiang teaches that the PAM-proximal 10-12 nucleotides, also known as the ‘seed region’ of the crRNA targeting sequence, are most critical for robust CRISPR/Cas9 binding. Specifically, Jiang discloses that mismatches in the seed region “severely impair or completely abrogate target DNA binding and cleavage, whereas close homology in the seed region often leads to off-target binding events even with many mismatches elsewhere,” i.e., in the PAM-distal 8-10 nucleotides. Jiang at 512. Similarly, Jiang teaches that “[p]erfect complementarity between the seed region of sgRNA and target DNA is necessary for Cas9-mediated DNA targeting and cleavage, whereas imperfect base pairing at the nonseed region is much more tolerated for target binding specificity. Id., citations omitted.

Accordingly, in some embodiments, a crRNA sequence used in the compositions and/or methods of the disclosure include one or more nucleotide substitutions, e.g., relative to any of SEQ ID NOs: 298-590, within the PAM-distal 8-10 nucleotides. In some embodiments, a crRNA sequence includes one nucleotide substitution, e.g., relative to any of SEQ ID NOs: 298-590, within the PAM-distal 8-10 nucleotides. In some embodiments, a crRNA sequence includes two nucleotide substitutions, e.g., relative to any of SEQ ID NOs: 298-590, within the PAM-distal 8-10 nucleotides. In some embodiments, a crRNA sequence includes three nucleotide substitutions, e.g., relative to any of SEQ ID NOs: 298-590, within the PAM-distal 8-10 nucleotides. In some embodiments, a crRNA sequence includes four nucleotide substitutions, e.g., relative to any of SEQ ID NOs: 298-590, within the PAM-distal 8-10 nucleotides. In some embodiments, a crRNA sequence includes five nucleotide substitutions, e.g., relative to any of SEQ ID NOs: 298-590, within the PAM-distal 8-10 nucleotides.

Accordingly, in some embodiments, a crRNA sequence used in the compositions and/or methods of the disclosure include one or more nucleotide substitutions, e.g., relative to any of SEQ ID NOs: 298-590, within the first 8 positions of the crRNA sequence. In some embodiments, a crRNA sequence includes one nucleotide substitution, e.g., relative to any of SEQ ID NOs: 298-590, within the first 8 positions of the crRNA sequence. In some embodiments, a crRNA sequence includes two nucleotide substitutions, e.g., relative to any of SEQ ID NOs: 298-590, within the first 8 positions of the crRNA sequence. In some embodiments, a crRNA sequence includes three nucleotide substitutions, e.g., relative to any of SEQ ID NOs: 298-590, within the first 8 positions of the crRNA sequence. In some embodiments, a crRNA sequence includes four nucleotide substitutions, e.g., relative to any of SEQ ID NOs: 298-590, within the first 8 positions of the crRNA sequence. In some embodiments, a crRNA sequence includes five nucleotide substitutions, e.g., relative to any of SEQ ID NOs: 298-590, within the first 8 positions of the crRNA sequence.

Similarly, in some embodiments, a crRNA sequence used in the compositions and/or methods of the disclosure include one or more nucleotide substitutions, e.g., relative to any of SEQ ID NOs: 298-590, within the first 10 positions of the crRNA sequence. In some embodiments, a crRNA sequence includes one nucleotide substitution, e.g., relative to any of SEQ ID NOs: 298-590, within the first 10 positions of the crRNA sequence. In some embodiments, a crRNA sequence includes two nucleotide substitutions, e.g., relative to any of SEQ ID NOs: 298-590, within the first 10 positions of the crRNA sequence. In some embodiments, a crRNA sequence includes three nucleotide substitutions, e.g., relative to any of SEQ ID NOs: 298-590, within the first 10 positions of the crRNA sequence. In some embodiments, a crRNA sequence includes four nucleotide substitutions, e.g., relative to any of SEQ ID NOs: 298-590, within the first 10 positions of the crRNA sequence. In some embodiments, a crRNA sequence includes five nucleotide substitutions, e.g., relative to any of SEQ ID NOs: 298-590, within the first 10 positions of the crRNA sequence.

Further, Jiang and Doudna postulates that base pairing of PAM-distal nucleotides at positions 14-17 of the crRNA targeting sequence are important for cleavage activity, following binding to the target sequence.

Accordingly, in some embodiments, a crRNA sequence used in the compositions and/or methods of the disclosure include one or more nucleotide substitutions, e.g., relative to any of SEQ ID NOs: 298-590, within nucleotide positions 1-3 and 8-10 of the crRNA sequence. In some embodiments, a crRNA sequence includes one nucleotide substitution, e.g., relative to any of SEQ ID NOs: 298-590, within nucleotide positions 1-3 and 8-10 of the crRNA sequence. In some embodiments, a crRNA sequence includes two nucleotide substitutions, e.g., relative to any of SEQ ID NOs: 298-590, within nucleotide positions 1-3 and 8-10 of the crRNA sequence. In some embodiments, a crRNA sequence includes three nucleotide substitutions, e.g., relative to any of SEQ ID NOs: 298-590, within nucleotide positions 1-3 and 8-10 of the crRNA sequence. In some embodiments, a crRNA sequence includes four nucleotide substitutions, e.g., relative to any of SEQ ID NOs: 298-590, within nucleotide positions 1-3 and 8-10 of the crRNA sequence. In some embodiments, a crRNA sequence includes five nucleotide substitutions, e.g., relative to any of SEQ ID NOs: 298-590, within nucleotide positions 1-3 and 8-10 of the crRNA sequence.

Similarly, in some embodiments, a crRNA sequence used in the compositions and/or methods of the disclosure includes one or more nucleotide substitutions, e.g., relative to any of SEQ ID NOs: 298-590, within nucleotide positions 1-3 and 8 of the crRNA sequence. In some embodiments, a crRNA sequence includes one nucleotide substitution, e.g., relative to any of SEQ ID NOs: 298-590, within nucleotide positions 1-3 and 8 of the crRNA sequence. In some embodiments, a crRNA. In some embodiments, a crRNA sequence includes two nucleotide substitutions, e.g., relative to any of SEQ ID NOs: 298-590, within nucleotide positions 1-3 and 8 of the crRNA sequence. In some embodiments, a crRNA. In some embodiments, a crRNA sequence includes three nucleotide substitutions, e.g., relative to any of SEQ ID NOs: 298-590, within nucleotide positions 1-3 and 8 of the crRNA sequence. In some embodiments, a crRNA. In some embodiments, a crRNA sequence includes four nucleotide substitutions, e.g., relative to any of SEQ ID NOs: 298-590, within nucleotide positions 1-3 and 8 of the crRNA sequence. In some embodiments, a crRNA.

In yet other embodiments, a crRNA sequence used in the compositions and/or methods of the disclosure include one or more nucleotide substitutions, e.g., relative to any of SEQ ID NOs: 298-590, throughout the entire sequence of the crRNA, e.g., as determined through experimentation. In some embodiments, a crRNA sequence used in the compositions and/or methods of the disclosure includes one nucleotide substitution, e.g., relative to any of SEQ ID NOs: 298-590, throughout the entire sequence of the crRNA, e.g., as determined through experimentation. In some embodiments, a crRNA sequence used in the compositions and/or methods of the disclosure includes two nucleotide substitutions, e.g., relative to any of SEQ ID NOs: 298-590, throughout the entire sequence of the crRNA, e.g., as determined through experimentation. In some embodiments, a crRNA sequence used in the compositions and/or methods of the disclosure includes three nucleotide substitutions, e.g., relative to any of SEQ ID NOs: 298-590, throughout the entire sequence of the crRNA, e.g., as determined through experimentation. In some embodiments, a crRNA sequence used in the compositions and/or methods of the disclosure includes four nucleotide substitutions, e.g., relative to any of SEQ ID NOs: 298-590, throughout the entire sequence of the crRNA, e.g., as determined through experimentation. In some embodiments, a crRNA sequence used in the compositions and/or methods of the disclosure includes five nucleotide substitutions, e.g., relative to any of SEQ ID NOs: 298-590, throughout the entire sequence of the crRNA, e.g., as determined through experimentation.

In some embodiments, the joint disease or condition is arthritis. In some embodiments, the arthritis is osteoarthritis.

In some embodiments, the administering includes intra-articular injection of the pharmaceutical composition into the joint of the subject. In some embodiments, the pharmaceutical composition is administered during surgery. In some embodiments, the pharmaceutical composition is administered after surgery. In some embodiments, the pharmaceutical composition is a controlled release pharmaceutical composition.

In some embodiments, the pharmaceutical composition includes one or more viral vectors, as described herein, collectively comprising the one or more nucleic acids. In some embodiments, the one or more viral vectors include a recombinant virus selected from a retrovirus, an adenovirus, an adeno-associated virus, a lentivirus, and a herpes simplex virus-1. In some embodiments, the one of more viral vectors include a recombinant adeno-associated virus (AAV). In some embodiments, the recombinant AAV is of serotype 5 (AAV5). In some embodiments, the recombinant AAV is of serotype 6 (AAV6).

In some embodiments, the one of more viral vectors include a first viral vector comprising a first nucleic acid, in the one or more nucleic acids, encoding the Cas9 protein, and a second viral vector comprising a second nucleic acid, in the one or more nucleic acids, encoding the at least one guide RNA. In some embodiments, the one of more viral vectors comprise a viral vector comprising a single nucleic acid, wherein the single nucleic acid encodes the Cas9 protein and the at least one guide RNA.

In some embodiments, the composition includes one or more liposomes collectively comprising the one or more nucleic acids. In some embodiments, the one or more nucleic acids are present in a naked state.

In some embodiments, the Cas9 protein is an S. pyogenes Cas9 polypeptide. In some embodiments, the Cas9 protein is an S. aureus Cas9 polypeptide.

### Methods of Treating Osteoarthritis and Other Diseases

The compositions and methods described herein can be used in a method for treating diseases. In an embodiment, they are for use in treating inflammatory joint disorders. They may also be used in treating other disorders as described herein and in the following paragraphs. In an aspect, the compositions and methods are used to treat osteoarthritis (OA).

In some embodiments, the present disclosure provides a method for the treatment or prevention of a joint disease or condition the method comprising introducing a gene-editing system, wherein the gene-editing system targets at least one locus related to joint function. In some embodiments, the joint disease is osteoarthritis. In an aspect, the method is used to treat a canine with osteoarthritis. In another aspect, the method is used to treat a mammal with degenerative joint disease. In some aspects, the method is used to treat a canine or an equine with a joint disease. In some aspects, the method is used to treat osteoarthritis, post-traumatic arthritis, post-infectious arthritis, rheumatoid arthritis, gout, pseudogout, auto-immune mediated arthritides, inflammatory arthritides, inflammation-mediated and immune-mediated diseases of joints.

In some embodiments, the method further comprises gene-editing a portion of a the joint synoviocytes to reduce or silence the expression of one or more of IL-1α, IL-1β, IL-4, IL-9, IL-10, IL-13, and TNF-α. In an aspect, the method further comprises gene-editing a portion of a the joint synoviocytes to reduce or silence the expression of one or more of IL-1α, IL-1β.

In an aspect, the method further comprises gene-editing, wherein the gene-editing comprises one or more methods selected from a CRISPR method, a TALE method, a zinc finger method, and a combination thereof.

In some aspects, the method further comprises delivering the gene-editing using an AAV vector, a lentiviral vector, or a retroviral vector. In a preferred embodiment, the method further comprises delivering the gene-editing using AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10. In some aspects, the AAV vector comprises a serotype selected from the group consisting of: AAV1, AAV5, AAV6, AAV6 (Y705F/Y731F/T492V), AAV8, AAV9, and AAV9 (Y731F).

### Pharmaceutical Compositions and Methods of Administration

The methods described herein include the use of pharmaceutical compositions comprising CRISPR gene (e.g., IL-1α and/or IL-1β) editing complexes as an active ingredient.

Depending on the method/route of administration, pharmaceutical dosage forms come in several types. These include many kinds of liquid, solid, and semisolid dosage forms. Common pharmaceutical dosage forms include pill, tablet, or capsule, drink or syrup, and natural or herbal form such as plant or food of sorts, among many others. Notably, the route of administration (ROA) for drug delivery is dependent on the dosage form of the substance in question. A liquid pharmaceutical dosage form is the liquid form of a dose of a chemical compound used as a drug or medication intended for administration or consumption.

In one embodiment, a composition of the present disclosure can be delivered to a subject subcutaneously (e.g., intra-articular injection), dermally (e.g., transdermally via patch), and/or via implant. Exemplary pharmaceutical dosage forms include, e.g., pills, osmotic delivery systems, elixirs, emulsions, hydrogels, suspensions, syrups, capsules, tablets, orally dissolving tablets (ODTs), gel capsules, thin films, adhesive topical patches, lollipops, lozenges, chewing gum, dry powder inhalers (DPIs), vaporizers, nebulizers, metered dose inhalers (MDIs), ointments, transdermal patches, intradermal implants, subcutaneous implants, and transdermal implants.

As used herein, “dermal delivery” or “dermal administration” can refer to a route of administration wherein the pharmaceutical dosage form is taken to, or through, the dermis (i.e., layer of skin between the epidermis (with which it makes up the cutis) and subcutaneous tissues). “Subcutaneous delivery” can refer to a route of administration wherein the pharmaceutical dosage form is to or beneath the subcutaneous tissue layer.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy)propane-co-sebacic-acid] (PCPP-SA) matrix, fatty acid dimer-sebacic acid (FAD-SA) copolymer, poly(lactide-co-glycolide)), polyglycolic acid, collagen, polyorthoesters, polyethyleneglycol-coated liposomes, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006 December; 79(3-4): 141-152.

The pharmaceutical compositions can be included in a container, kit, pack, or dispenser together with instructions for administration.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1. Reducing IL-1 Expression by CRISPR Gene-Engineering in a Mouse Model of Osteoarthritis

Sixty C57B mice are selected and distributed into four groups of fifteen mice each. The DMM surgical method is used to induce OA in each of the mice. Once the mice have developed OA, the mice are treated as follows:

Group 1: Direct injection into the OA joint a CRISPR AAV vector engineered to target IL-1α and IL-1β, and silence or reduce the expression of IL-1 protein.

Group 2: Direct injection into the OA joint a CRISPR AAV vector engineered with a “nonsense” payload that will not affect an IL-1 production; a negative control.

Group 3: Direct injection into the OA joint a CRISPR AAV vector engineered to target IL-1Ra, and silence or reduce the expression of IL-1Ra protein.

Group 4: Direct injection into the OA joint sterile buffered saline; a control for the injection process.

The mice are monitored before and after treatment to assess effects on their locomotion, and exploratory activities. Mechanical sensitivity and changes to the gait are also monitored. Allodynia and hind limb grip force may also be monitored.

After about eight weeks, the animals are sacrificed and the OA joint tissue assessed for gross histopathology, and IL-1 expression by IHC. Biomarkers of inflammation are also assessed, for example, MMP-3 expression in the OA joint.

Group 1 mice, treated with a CRISPR AAV vector engineered to target IL-1α and IL-1β, and silence or reduce the expression of IL-1 protein, will show reduced levels of IL-1 by IHC, tissue regeneration by histopathology, and lower levels of inflammation biomarkers than any of the three other Groups. Group 3 mice will show relatively higher levels of inflammation biomarker than any of the other three groups.

### Example 2. Assessing Guide Cutting Efficiency Against Mouse IL1A and IL1B

In Vitro Cleavage Assay

CRISPR guide RNA's (Phosphorothionate-modified sgRNA, Table 4) were designed against Exon 4 of Il1a and Exon 4 of Il1b (Il1a-201 ENSMUST00000028882.1 and Il1b-201 ENSMUST00000028881.13; see Table 3 for target sequences on Exon 4 of Il1a and Exon 4 of Il1b). C57BL/6 mouse genomic DNA was used to amplify Exon 4 of Il1a and Il1b by PCR (Phusion High-Fidelity DNA polymerase, NEB cat #M0530S) Il1a primer fwd: CATTGGGAGGATGCTTAGGA (SEQ ID NO:620), Il1a primer rev: GGCTGCTTTCTCTCCAACAG (SEQ ID NO:621), Il1b primer fwd: AGGAAGCCTGTGTCTGGTTG (SEQ ID NO:622), Il1b primer rev: TGGCATCGTGAGATAAGCTG (SEQ ID NO:623). Amplicons were PCR purified (QiaQuick PCR purification kit cat #28106). Guide cutting efficiency was determined using an in vitro cleavage assay using 100 ng purified PCR product, 200 ng modified guide RNA (Sigma Aldrich) and 0.5 μg TrueCut Spy Cas9 protein V2 (Invitrogen A36498) or 0.5 μg Gene Snipper NLS Sau Cas9 (BioVision Cat #M1281-50-1). The two types of Cas9, S. pyogenes Cas9 and S. aureus Cas9, were compared for their editing capabilities. A 2% agarose gel was used for a qualitative readout of the cleavage assay.

Editing Cell Lines

CRISPR guide RNA's (Phosphorothionate-modified sgRNA, Table 3) were designed against Exon 4 of Il1a and Exon 4 of Il1b (Il1a-201 ENSMUST00000028882.1 and Il1b-201 ENSMUST00000028881.13). Guide RNA cutting efficiency was determined in a pool of J774.2 and NIH3T3 cells using Sanger sequencing and Synthego ICE (see, e.g., Inference of CRISPR Edits from Sanger Trace Data, Hsiau T, Maures T, Waite K, Yang J et al. biorxiv. 2018, which is incorporated by reference herein for all purposes), or TIDE (see, e.g., Easy quantitative assessment of genome editing by sequence trace decomposition, Brinkman E, Chen T, Amendola M and Van Steensel B. Nucleic Acids res 2014, which is incorporated by reference herein for all purposes) web tools to calculate percent editing. The experiment also compared the efficiency of S. pyogenes Cas9 and S. aureus Cas9. The cells were electroporated (Amaxa 4D Nucleofector unit, Lonza) with 5 μg TrueCut Spy Cas9 protein V2 (Invitrogen A36498) or 5 μg EnGen Sau Cas9 protein (NEB M0654T) with 100 pmol modified guide RNA (Sigma Aldrich). SF nucleofector solution and programme CM139 was used for J774.2 cells and SG nucleofector solution and programme EN158 was used for NIH3T3 cells. A cell pellet was taken 3 days' post electroporation and gDNA was extracted from each pool (Qiagen, DNeasy blood and tissue kit, 69506). Exon 4 of Il1a or Il1b was amplified in the appropriate pool by PCR (Phusion High-Fidelity DNA polymerase, NEB, cat #M0530S). Il1a primer fwd: TGGTTTCAGGAAAACCCAAG (SEQ ID NO:624), Il1a primer rev: GCAGTATGGCCAAGAAAGGA (SEQ ID NO:625), Il1b primer fwd: AGGAAGCCTGTGTCTGGTTG (SEQ ID NO:622), Il1b primer rev: CTGGGCAAGAACATTGGATT (SEQ ID NO:626). Amplicons were subjected to Sanger sequencing, and analyzed using either the Synthego ICE or TIDE web tools to determine the absence of wild type sequence in each clone and the presence of indels resulting in a frameshift in the cDNA sequence.

TABLE 3
Target Il1a and Il1b Sequences

| | Guide | | | | | |
|---|---|---|---|---|---|---|
| Identifier | ID | Gene | Exon | Cas9 | Target Sequence 5′-3′ | PAM |
| SEQ ID | sg43 | Il1a | 4 | S.pyogenes | GTATCAGCAACGTCAAGCAA | CGG |
| NO: 7 | | | | | | |
| SEQ ID | sg44 | Il1a | 4 | S.pyogenes | CTGCAGGTCATCTTCAGTGA | AGG |
| NO: 8 | | | | | | |
| SEQ ID | sg45 | Il1a | 4 | S.pyogenes | TATCAGCAACGTCAAGCAAC | GGG |
| NO: 9 | | | | | | |
| SEQ ID | sg46 | Il1a | 4 | S.pyogenes | GCCATAGCTTGCATCATAGA | AGG |
| NO: 10 | | | | | | |
| SEQ ID | sg47 | Il1b | 4 | S.pyogenes | CATCAACAAGAGCTTCAGGC | AGG |
| NO: 11 | | | | | | |
| SEQ ID | sg48 | Il1b | 4 | S.pyogenes | TGCTCTCATCAGGACAGCCC | AGG |
| NO: 12 | | | | | | |
| SEQ ID | sg49 | Il1b | 4 | S.pyogenes | GCTCATGTCCTCATCCTGGA | AGG |
| NO: 13 | | | | | | |
| SEQ ID | sg50 | Il1b | 4 | S.pyogenes | CCTCATCCTGGAAGGTCCAC | GGG |
| NO: 14 | | | | | | |
| SEQ ID | sg51 | Il1a | 4 | S.aureus | TTACTCCTTACCTTCCAGATC | ATGGGT |
| NO: 15 | | | | | | |
| SEQ ID | sg52 | Il1a | 4 | S.aureus | GAAACTCAGCCGTCTCTTCTT | CAGAAT |
| NO: 16 | | | | | | |
| SEQ ID | sg53 | Il1a | 4 | S.aureus | CAACTTCACCTTCAAGGAGAG | CCGGGT |
| NO: 17 | | | | | | |
| SEQ ID | sg54 | Il1b | 4 | S.aureus | GTGTCTTTCCCGTGGACCTTC | CAGGAT |
| NO: 18 | | | | | | |
| SEQ ID | sg55 | Il1b | 4 | S.aureus | CACAGCTTCTCCACAGCCACA | AGTAGT |
| NO: 19 | | | | | | |
| SEQ ID | sg56 | Il1b | 4 | S.aureus | GTGCTGCTGCGAGATTTGAAG | CTGGAT |
| NO: 20 | | | | | | |

TABLE 4
CRISPR Guide RNA’s.

| | Guide | | | | | |
|---|---|---|---|---|---|---|
| Identifier | ID | Gene | Exon | Cas9 | cRNA Sequence 5’-3’ | PAM |
| SEQ ID | sg43 | Il1a | 4 | S.pyogenes | GUAUCAGCAACGUCAAGCAA | CGG |
| NO: 21 | | | | | | |
| SEQ ID | sg44 | Il1a | 4 | S.pyogenes | CUGCAGGUCAUCUUCAGUGA | AGG |
| NO: 22 | | | | | | |
| SEQ ID | sg45 | Il1a | 4 | S.pyogenes | UAUCAGCAACGUCAAGCAAC | GGG |
| NO: 23 | | | | | | |
| SEQ ID | sg46 | Il1a | 4 | S.pyogenes | GCCAUAGCUUGCAUCAUAGA | AGG |
| NO: 24 | | | | | | |
| SEQ ID | sg47 | Il1b | 4 | S.pyogenes | CAUCAACAAGAGCUUCAGGC | AGG |
| NO: 25 | | | | | | |
| SEQ ID | sg48 | Il1b | 4 | S.pyogenes | UGCUCUCAUCAGGACAGCCC | AGG |
| NO: 26 | | | | | | |
| SEQ ID | sg49 | Il1b | 4 | S.pyogenes | GCUCAUGUCCUCAUCCUGGA | AGG |
| NO: 27 | | | | | | |
| SEQ ID | sg50 | Il1b | 4 | S.pyogenes | CCUCAUCCUGGAAGGUCCAC | GGG |
| NO: 28 | | | | | | |
| SEQ ID | sg51 | Il1a | 4 | S.aureus | UUACUCCUUACCUUCCAGAUC | ATGGGT |
| NO: 29 | | | | | | |
| SEQ ID | sg52 | Il1a | 4 | S.aureus | GAAACUCAGCCGUCUCUUCUU | CAGAAT |
| NO: 30 | | | | | | |
| SEQ ID | sg53 | Il1a | 4 | S.aureus | CAACUUCACCUUCAAGGAGAG | CCGGGT |
| NO: 31 | | | | | | |
| SEQ ID | sg54 | Il1b | 4 | S.aureus | GUGUCUUUCCCGUGGACCUUC | CAGGAT |
| NO: 32 | | | | | | |
| SEQ ID | sg55 | Il1b | 4 | S.aureus | CACAGCUUCUCCACAGCCACA | AGTAGT |
| NO: 33 | | | | | | |
| SEQ ID | sg56 | Il1b | 4 | S.aureus | GUGCUGCUGCGAGAUUUGAAG | CTGGAT |
| NO: 34 | | | | | | |

Each cRNA (see, e.g., Table 4) was synthesized as a single guide RNA consisting of the cRNA sequences above fused to the tracrRNA sequences below (see, e.g., SEQ ID Nos: 35-36). In certain embodiments, an A< >U flip is used to increase guide RNA activity.

SauCas9:
(SEQ ID NO: 35)
GUUAUAGUACUCUGGAAACAGAAUCUACUAAAACAAGGCAAAAUGCCGU
GUUUAUCUCGUCAACUUGUUGGCGAGAUUUUU
Spy Cas9:
(SEQ ID NO: 36)
GUUAUAGAGCUAGAAAUAGCAAGUUAAAAUAAGGCUAGUCCGUUAUCAA
CUUGAAAAAGUGGCACCGAGUCGGUGCUUUUUU

In Vitro Cleavage Assay

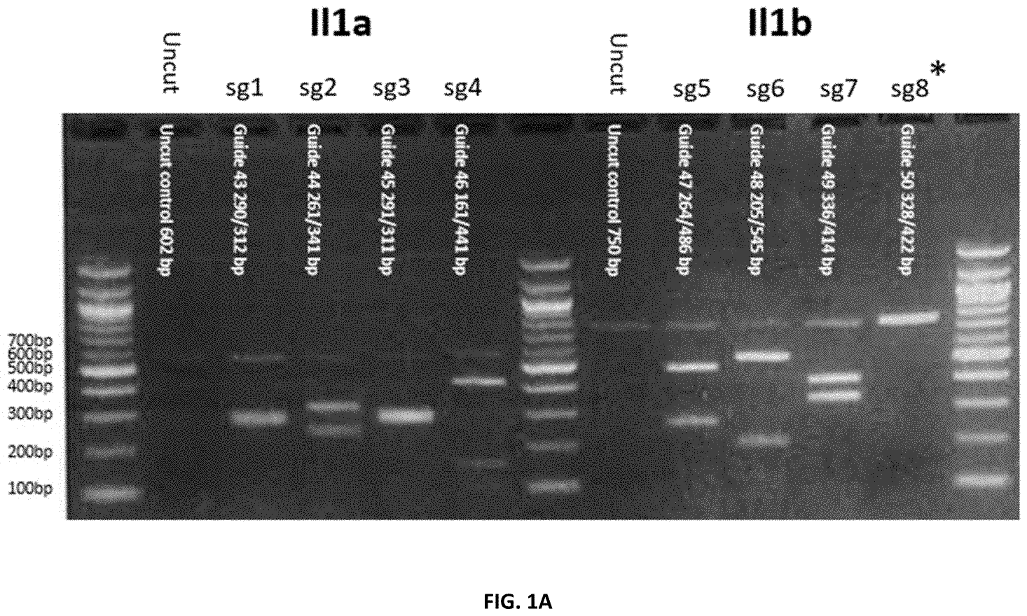

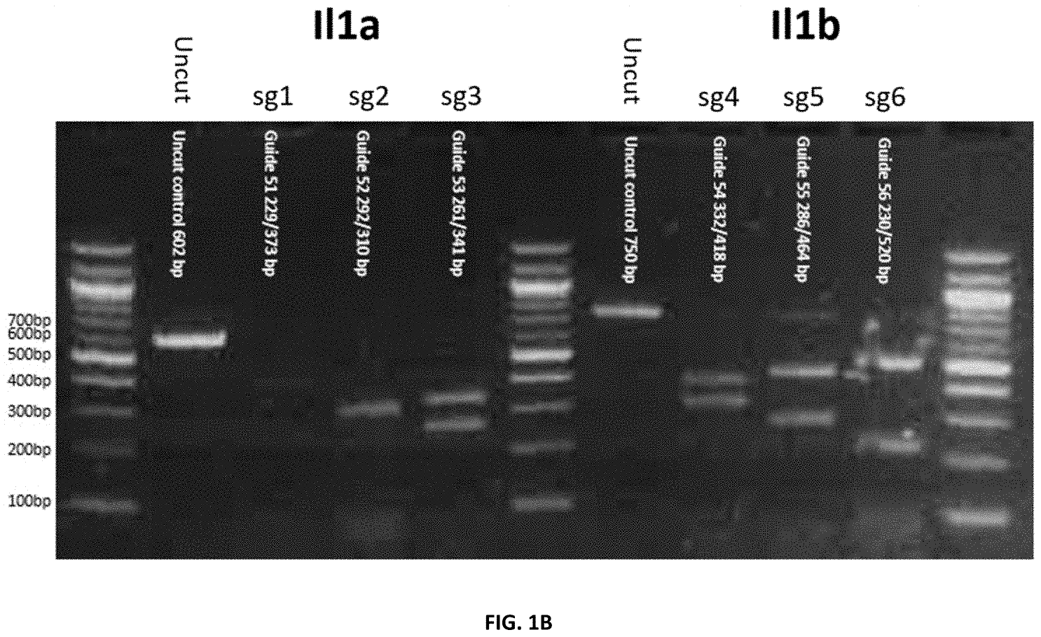

FIG. 1A illustrates agarose gel electrophoresis analysis of 100 ng mouse DNA, cleaved by 0.5 μg Spy Cas9 and 200 ng modified guide RNA's 43-46 for Il1a gene and 47-50 for IL1B. DNA is cut at a specific site by the Cas9 using the guide RNA to create a predictable band pattern on the agarose gel compared to the uncut control (without wishing to be bound by any particular theory, the agarose gel electrophoresis for sg8* appears to show a failed synthesis).

FIG. 1B illustrates agarose gel electrophoresis analysis of 100 ng mouse DNA, cleaved by 0.5 μg Sau Cas9 and 200 ng modified guide RNA's 51-53 for Il1a gene and 54-56 for Il1b. DNA is cut at a specific site by the Cas9 using the guide RNA to create a predictable band pattern on the agarose gel compared to the uncut control.

Editing Cell Lines

Genomic DNA was extracted from the edited pools and the Il1a or Il1b exon 4 was PCR amplified in the appropriate pools. The PCR products were sent for sanger sequencing and then deconvoluted using TIDE or Synthego ICE software. Synthego ICE was used to deconvolute the Spy Cas9 pools. The software can determine the patterns of editing in each pool based on the guide RNA sequence and PAM site. It can distinguish between editing which has caused an in frame deletion that could lead to a truncated functional protein, and editing which has causes a frameshift mutation which will lead to a true knockout. The SauCas9 pools were analysed with TIDE because Synthego ICE software cannot deconvolute SauCas9 editing. TIDE analysis works in a similar way to ICE by determining patterns of editing in a pool based on the guide RNA and PAM site. However, rather than giving a true knockout score, it gives an editing efficiency score, which cannot distinguish between in frame and frameshift editing patterns. Therefore, editing efficiency scores may over represent the guide RNA's ability to knockout a protein. SpyCas9 is the standard protein used in CRISPR gene editing. However, it is 4101 bp compared to Sau Cas9 which is 3156 bp. Due to the size limitations of packaging some viruses, such as AAV, it was decided to compare the editing capabilities of SauCas9 and SpyCas9 to see whether the smaller Sau Cas9 could be used in the vector being designed for this project.

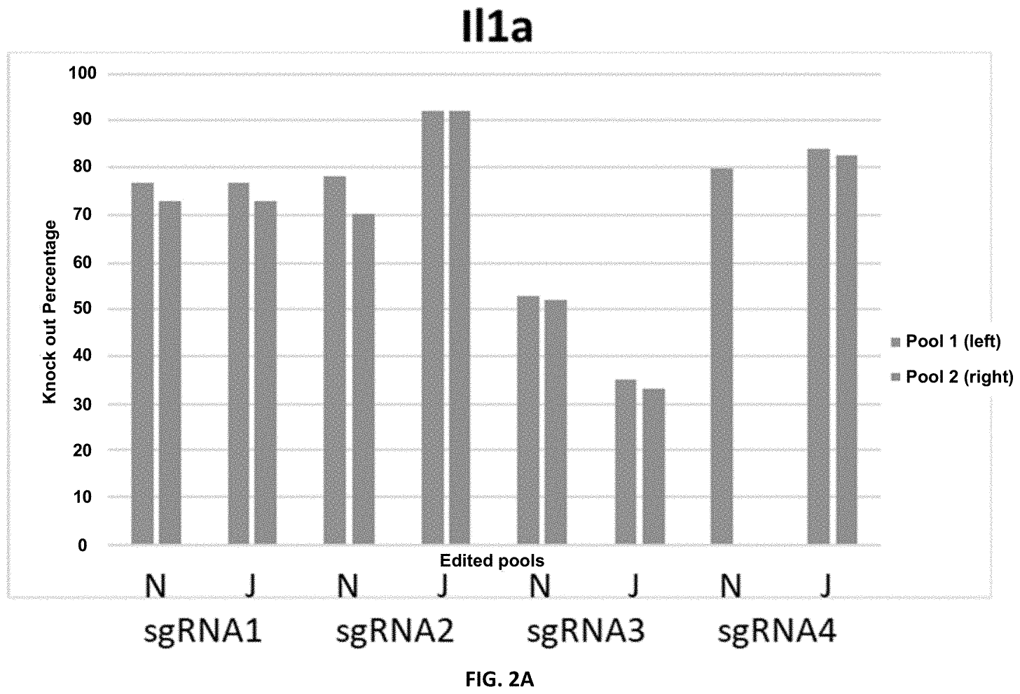

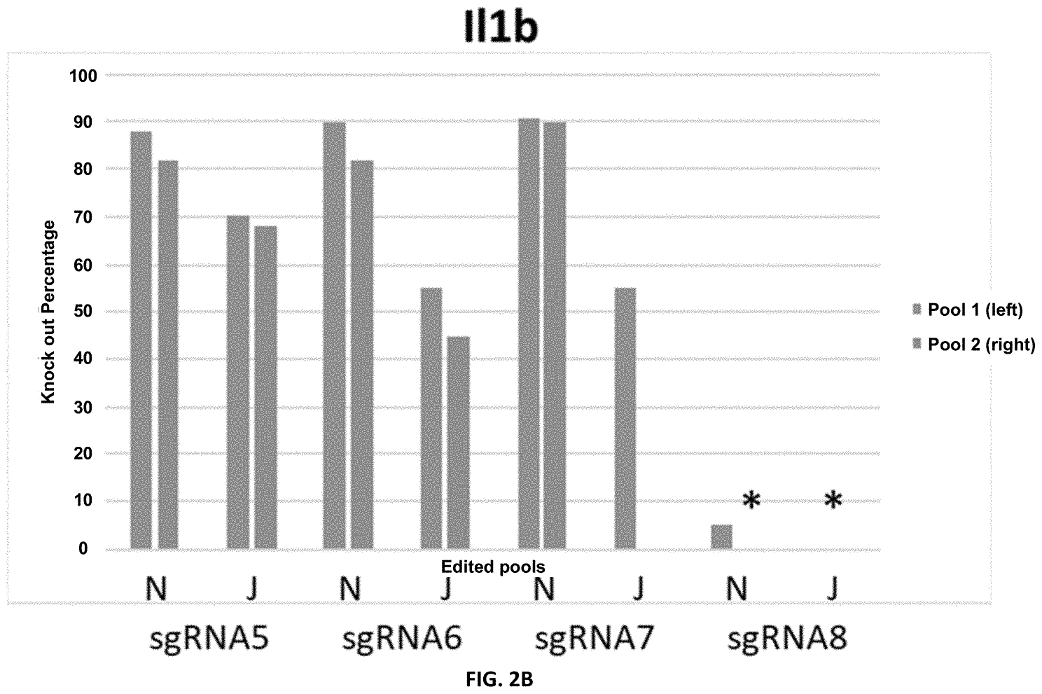

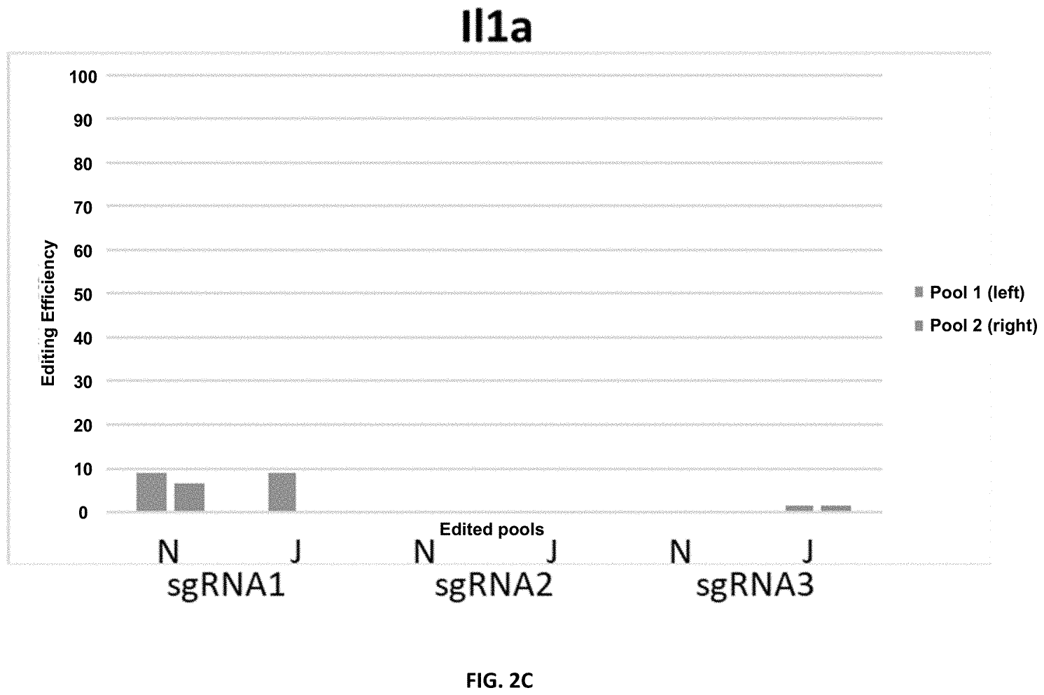

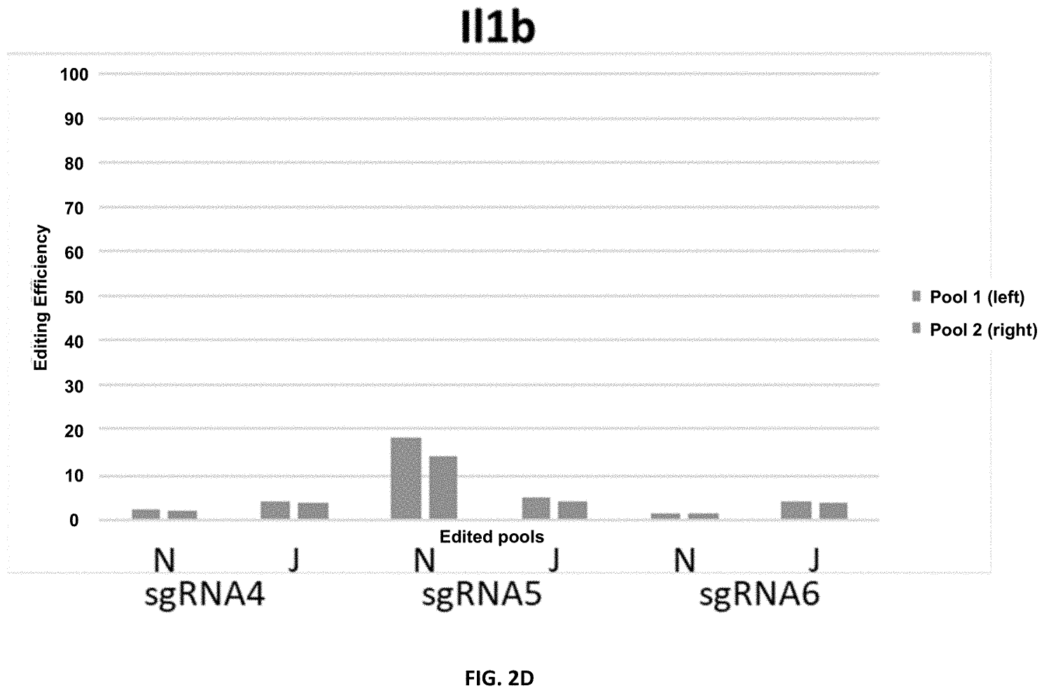

FIGS. 2A-2D illustrate graphs displaying editing efficiencies of Spy Cas9 (FIGS. 2A and 2B) and SauCas9 (FIGS. 2C and 2D) used with a range of guide RNA's in J774.2 (“J”) and NIH3T3 (“N”) cells. Editing efficiencies were determined using Synthego ICE or TIDE sanger deconvolution software. FIG. 2A: knock out efficiency of Il1a using guide RNA 43-46 with SpyCas9 in J774.2 and NIH3T3. Synthego ICE was used to deconvolute the sanger sequence trace and determine knock out efficiency. FIG. 2B: knock out efficiency of Il1b using guide RNA 47-50 with SpyCas9 in J774.2 and NIH3T3; without wishing to be bound by any particular theory, the data for sgRNA8 appears to show a failed synthesis. Synthego ICE was used to deconvolute the sanger sequence trace and determine knock out efficiency. FIG. 2C: knock out efficiency of Il1a using guide RNA 51-53 with saCas9 in J774.2 and NIH3T3. TIDE was used to deconvolute the sanger sequence trace and determine the editing efficiency. FIG. 2D: knock out efficiency of Il1b using guide RNA 54-56 with Sau Cas9 in J774.2 and NIH3T3. TIDE was used to deconvolute the sanger sequence trace and determine the editing efficiency.

### Example 3. Reducing IL-1β Expression by CRISPR Gene-Engineering in a Mouse Uric Acid Model

Time Course Experiment to Determine Optimal Pre-Treatment Time

A pilot experiment is performed to determine optimal pre-treatment time of mice with virus prior to challenging the mice with uric acid. Mice are injected with GFP-labeled AAV5 vector into the knee joint. Viral load is then quantified by PCR and location of viral infection is quantified by histology at 3, 5, and 7 days after infection. A treatment time that yields robust expression of virus inside the joint is selected as the optimal lead time for injecting viral vectors into the mice for the experiments to determine the reduction of IL-1b in a mouse uric acid model by a CRISPR AAV vector engineered to target IL-1b and silence or reduce expression of IL-1b.

Experiment to Confirm CRISPR AAV (AAV-spCas9) Knockdown of lL-1b Expression and Treatment Effect in Uric Acid Model

Mice are selected and distributed into three groups:

Group 1: mice injected with a CRISPR AAV vector (AAV-spCas9) engineered to target IL-1b, and silence or reduce expression of IL-1 protein,

Group 2: mice injected with “scrambled” guide RNA/Cas9 (AAV-spCas9), a CRISPR AAV vector engineered with a payload that will not affect IL-1 production, and Group 3: mice injected with saline.

The mice are then challenged with uric acid after an optimal pre-treatment time. Within 24 hours of injection with uric acid, the animals are sacrificed and the joint tissue is analyzed for cytokine expression (e.g., assessed for IL-1 expression by IHC). The joint tissue may also be assessed for gross histopathology and for expression of biomarkers of inflammation.

Group 1 mice treated with a CRISPR AAV vector engineered to target IL-1b, and silence or reduce the expression of IL-1 protein, will show reduced levels of IL-1 by IHC and lower levels of inflammation biomarkers than any of the two other groups.

### Example 4. Time Course Study of Intra-Articular Injection of AAV in Mice

A study was conducted to evaluate the time course for injecting AAV into the joint of male C57BL/6 mice.

Materials & Methods

Test Article Identification and Preparation—The eGFP AAVPrime™ Purified Adeno-associated Viral Particles: GFP-tagged AAV5 GeneCopoeia™, catalogue No. AB201, lot No. GC08222K1902, 1.18×10¹³ Genome Copies/mL) and AAV6 (GeneCopoeiam, catalogue No. AB401, lot No. GC09242K1905, 5.47×10¹² Genome Copies/mL) we supplied. AAV-particles were shipped on dry ice and were stored at −80° C. immediately upon receipt. Just prior to dosing, the AAV-particles were reconstituted in phosphate buffered saline (PBS without calcium and magnesium: Corning, lot No. 11419005) for IA dosing at 10 μL per knee. See the study protocol (Appendix A) for additional details of test article preparation, storage, and handling.

Test System Identification—Male C57BL/6 mice (N=30) that were 8 to 10 weeks old were obtained from The Jackson Laboratory (Bar Harbor, Me.). The mice weighed approximately 24 to 29 grams (mean of 26 g) at enrollment on study day 0. The animals were identified by a distinct mark at the base of the tail delineating group and animal number. After randomization, all cages were labeled with protocol number, group numbers, and animal numbers with appropriate color-coding (Appendix A).

Environment & Husbandry—Upon arrival, the animals were housed 3 to 5 per cage in polycarbonate cages with wood chip bedding and suspended food and water bottles. The mice were housed either in shoebox cages (static airflow, approximately 70 in2 floor space) with filter tops or in individually ventilated pie cages (passive airflow, approximately 70-75 in2 floor space). Animal care including room, cage, and equipment sanitation conformed to the guidelines cited in the Guide for the Care and Use of Laboratory Animals (8th Edition). National Research Council, National Academy of Sciences, Washington, D.C., 2011, which is incorporated by reference herein in its entirety for all purposes.

The animals were acclimated for 4 days prior to being paced in the study. An attending veterinarian was on site or on call during the live phase of the study. No concurrent medications were given.

During the acclimation and study periods, the animals were housed in a laboratory environment with temperatures ranging 19° C. to 25° C. and relative humidity of 30% to 70%. Automatic timers provided 12 hours of light and 12 hours of dark. The animals were allowed access ad libitum to Envigo Teklad 8640 diet and fresh municipal tap water.

Experimental Design—On study day 0, the mice were randomized by body weight into treatment groups. Following randomization, the animals were dosed by intra-articular (IA) injection as indicated in Table 5. Animal body weights were measured as described in section 8.5.1. The mice were euthanized for necropsy and tissue collection at 3 time points (days 3, 5, and 7) as described below in the section titled ‘Necropsy Specimens’.

TABLE 5
Group and Treatment Information

| | | | Dose Level | Dose | Dose Conc. | Dose | |
|---|---|---|---|---|---|---|---|
| Group | N | Treatment | (particles) | Vol. | (particles/ml) | Route | Regimen |
| 1 | 30 | GFP-tagged | 5 × 10⁹ | 10 μL | 5 × 10₁₁/mL | IA (right | 1 × (Day 0) |
| | | AAV5 | | | | knee) | |
| | | GFP-tagged | 5 × 10⁹ | 10 μL | 5 × 10¹¹/mL | IA (left | 1 × (Day 0) |
| | | AAV6 | | | | knee) | |

Observations, Measurements, and Specimens

Body Weight Measurements—The mice were weighed for randomization on study day 0 and again on days 1, 3, 5, and 7. Body weight measurements can be found in Table 7.

Necropsy Specimens—The mice were necropsied on study days 3, 5, and 7 as indicated in Table 6.

TABLE 6
Necropsy Schedule

| Group | Animal No. | Time-point(s) |
|---|---|---|
| 1 | 1-10 | Day 3 |
| 1 | 11-20 | Day 5 |
| 1 | 21-30 | Day 7 |

At necropsy, the mice were bled to exsanguination via cardiac puncture followed by cervical dislocation. Right and left knees were harvested from all animals. The skin and muscle were removed from the joints while keeping the joint capsule intact. Joints were flash-frozen separately in 15-mL conical tubes labeled with only mouse number, day of collection, and right or left leg. Knee joints were stored frozen at −80° C. for shipment.

Animal Disposition—Animal carcasses were disposed of according to BBP SOPs.

Specimen and Raw Data Storage—Specimens (right and left knee joints), study data, and reports were delivered during or at the completion of the study.

Statement of Effect of Deviations on the Quality and Integrity of the Study—There were no deviations from the study protocol.

Results/Conclusions

On study day 0, male C57BL/6 mice received IA injections of GFP-tagged AAV5 (5×10⁹ particles, 10 μL) into right knees and IA injections of GFP-tagged AAV6 (5×10⁹ particles, 10 μL) into left knees. The animals were weighed on study days 0, 1, 3, 5, and 7. Necropsies were performed on study day 3 (animals 1-10), day 5 (animals 11-20), and day 7 (animals 21-30), and right and left knee joints were collected for shipment. The live portion of this study was completed successfully including animal weighing, dosing, and biological sample collection. All animals survived to study termination.

### REFERENCES

Guide for the Care and Use of Laboratory Animals (8th Edition). National Research Council, National Academy of Sciences, Washington, D.C., 2011, which is incorporated by reference herein in its entirety for all purposes.

TABLE 7
Body Weight and Dose Calculation Data (MTC-UCM-1)

| Treatment | | | | | | |
|---|---|---|---|---|---|---|
| Group | | | | | | |
| Group [symbol] | Day 0 | Day 0 | Day 1 | | Day 3 | |
| C57B1/6 | Body Wt. | Dose Vol. | Body Wt. | Body Wt. % Δ | Body Wt. | Body Wt. % Δ |
| IA, 1 × (D 0) | (g) | 10 μl (ml) | (g) | Baseline | (g) | Baseline |
| 1 | 26.68 | 0.01 | 26.53 | −0.6% | 26.22 | −1.7% |
| 2 | 27.65 | 0.01 | 27.55 | −0.4% | 26.54 | −4.0% |
| 3 | 28.64 | 0.01 | 27.97 | −2.3% | 28.57 | −0.2% |
| 4 | 28.13 | 0.01 | 27.60 | −1.9% | 27.63 | −1.8% |
| 5 | 26.18 | 0.01 | 26.07 | −0.4% | 2 [symbol] .87 | −1.2% |
| 6 | 26.38 | 0.01 | 26.02 | −1.4% | 26.30 | −0.3% |
| 7 | 29.13 | 0.01 | 29.11 | −0.1% | 28.83 | −1.0% |
| 8 | 24.22 | 0.01 | 23.90 | −1.3% | 23.51 | −2.9% |
| 9 | 28.40 | 0.01 | 24.97 | −1.7% | 24.49 | −3.6% |
| 10 | 24.85 | 0.01 | 24.21 | −2.6% | 23.86 | −1.0% |
| 11 | 27.76 | 0.01 | 27.26 | −1.8% | 28.02 | 0.9% |
| 12 | 25.23 | 0.01 | 24.77 | −1.8% | 24.90 | −1.3% |
| 13 | 24.92 | 0.01 | 24.45 | −1.9% | 24.59 | −1.3% |
| 14 | 24.33 | 0.01 | 24.19 | −0.6% | 23.86 | −1.9% |
| 15 | 23.82 | 0.01 | 23.81 | 0.0% | 23.26 | −2.4% |
| 16 | 24.83 | 0.01 | 24.39 | −1.8% | 24.15 | −2.7% |
| 17 | 25.94 | 0.01 | 25.93 | 0.0% | 26.28 | 1.3% |
| 18 | 27.21 | 0.01 | 27.44 | 0.8% | 27.60 | 1.4% |
| 19 | 25.61 | 0.01 | 25.17 | −1.7% | 25.22 | −1.5% |
| 20 | 27.81 | 0.01 | 27.26 | −2.0% | 26.99 | −2.9% |
| 21 | 26.63 | 0.01 | 26.63 | 0.0% | 26.71 | 0.3% |
| 22 | 26.96 | 0.01 | 27.40 | 1.6% | 2 [symbol] .75 | −4.5% |
| 23 | 27.69 | 0.01 | 27.21 | −1.7% | 27.22 | −1.7% |
| 24 | 25.90 | 0.01 | 25.71 | −0.7% | 2 [symbol] .45 | −1.7% |
| 25 | 24.03 | 0.01 | 24.11 | 0.3% | 2 [symbol] .40 | −2.6% |
| 26 | 27.60 | 0.01 | 26.67 | −3.4% | 27.00 | −2.2% |
| 27 | 27.87 | 0.01 | 27.59 | −1.0% | 27.22 | −2.3% |
| 28 | 24.43 | 0.01 | 24.25 | −0.7% | 24.14 | −1.2% |
| 29 | 26.75 | 0.01 | 26.02 | −2.7% | 26.46 | −1.1% |
| 30 | 25.93 | 0.01 | 25.99 | 0.2% | 25.80 | −0.5% |
| Mean | 26.28 | | 26.01 | −1.0% | 25.86 | −1.6% |
| SE | 0.27 | | 0.27 | 0.2% | 0.29 | 0.3% |

| Treatment | | | | | |
|---|---|---|---|---|---|
| Group | | | | | |
| Group [symbol] | Day 5 | | Day 7 | | Change in |
| C57B1/6 | Body Wt. | Body Wt. % Δ | Body Wt. | Body Wt. % Δ | Body weight |
| IA, 1 × (D 0) | (g) | Baseline | (g) | Baseline | from Baseline (g) |
| 1 | | | | | −0.46 |
| 2 | | | | | −1.11 |
| 3 | | | | | −0.07 |
| 4 | | | | | −0.50 |
| 5 | | | | | −0.31 |
| 6 | | | | | −0.08 |
| 7 | | | | | −0.30 |
| 8 | | | | | −0.71 |
| 9 | | | | | −0.91 |
| 10 | | | | | −0.99 |
| 11 | 28.62 | 3.1% | | | 0. [symbol] 6 |
| 12 | 25.04 | −0.8% | | | −0.19 |
| 13 | 24.94 | 0.1% | | | 0.02 |
| 14 | 23.68 | −2.7% | | | −0.65 |
| 15 | 23.71 | −0.5% | | | −0.11 |
| 16 | 24.26 | −2.3% | | | −0.57 |
| 17 | 26.15 | 0.8% | | | 0.21 |
| 18 | 27.88 | 2.5% | | | 0.67 |
| 19 | 25.64 | 0.1% | | | 0.03 |
| 20 | 27.71 | −0.4% | | | −0.10 |
| 21 | 26.44 | −0.7% | 26.70 | 0.3% | 0.07 |
| 22 | 25.7 [symbol] | −4.5% | 25.75 | −4.5% | −1.21 |
| 23 | 27.53 | −0. [symbol] % | 27.33 | −1. [symbol] % | −0.36 |
| 24 | 23.61 | −1.1% | 26.00 | 0.4% | 0.10 |
| 25 | 23.76 | −1.1% | 24.10 | 0.3% | 0.07 |
| 26 | 27.44 | −0.6% | 27.18 | −1.5% | −0.42 |
| 27 | 27.09 | −2.8% | 27.30 | −2.0% | −0.57 |
| 28 | 24.50 | 0.3% | 24.76 | 1.4% | 0.33 |
| 29 | 26.48 | −1.0% | 26.48 | −1. [symbol] % | −0.27 |
| 30 | 25.73 | −0.8% | 25.89 | 0.2% | −0.04 |
| Mean | 25.90 | −0.6% | 26.15 | −0.8% | −0.25 |
| SE | 0.33 | 0.4% | 0.34 | 0.5% | 0.09 |

[symbol]

indicates data missing or illegible when filed

Protocol

Test System

Number of animals: 33 (30+3 extra)

### Species/Strain or Breed: C57BL/6

### Vendor: Jackson

Age/Wt at Arrival: 8-10 weeks old (˜20 grams)

### Gender: Male

Age/Wt Range at Study Initiation: At least 9 weeks by study initiation
Acclimation: Will be acclimated for at least 3 days after arrival at

### BBP

Housing: 3-5 animals/cage

### Study Calendar

| [symbol] | [symbol] | [symbol] | [symbol] | [symbol] | [symbol] | [symbol] |
|---|---|---|---|---|---|---|
| Week 1 | Week 1 | Week 1 | Week 1 | Week 1 | Week 1 | Week 1 |
| Distribute | | | | Weigh & | Weigh | |
| animals on | | | | Randomize. IA | | |
| arrival into | | | | Injections | | |
| groups for [symbol] | | | | | | |
| Week 2 | Week 2 | Week 2 | Week 2 | Week 2 | Week 2 | Week 2 |
| Weigh, | | Weigh, | | Weigh, | | |
| Necropsy | | Necropsy | | Necropsy | | |
| Animals 1-10 | | Animals 11- [symbol] | | Animals 21- [symbol] | | |

[symbol]

indicates data missing or illegible when filed

Materials

| [symbol] | [symbol] | [symbol] |
|---|---|---|
| [symbol] | [symbol] | [symbol] |

[symbol]

indicates data missing or illegible when filed

Test Article and Vehicle Information

Unformulated Test Article Storage Conditions—GFP-tagged AAV5 (Group 1): −80C; GFP-tagged AAV6 (Group 1): −80° C.

Vehicle Information—GFP-tagged AAV5 (Group 1): PBS (w/o Ca & Mg); GFP-tagged AAV6 (Group 1): PBS (w/o Ca & Mg).

Test Article Formulation Instructions & Calculations—GFP-tagged AAV5 (Group 1): Dilute stock to appropriate concentration using PBS; GFP-tagged AAV6 (Group 1): Dilute stock to appropriate concentration using PBS.

Dosing Formulations and Vehicle Storage & Stability—GFP-tagged AAV5 (Group 1): Dilute just prior to injecting; GFP-tagged AAV6 (Group 1): Dilute just prior to injecting.

Disposition of Test Articles Following Dosing—GFP-tagged AAV5 (Group 1): Discard formulations, retain stock solution for future studies; GFP-tagged AAV6 (Group 1): Discard formulations, retain stock solution for future studies.

Live Phase Deliverables

[symbol]

| | | | |
|---|---|---|---|
| [symbol] | [symbol] | [symbol] | [symbol] |

| | | |
|---|---|---|
| Body Weight | Day 0, 1, 3, 5, 7 | All |

[symbol]

indicates data missing or illegible when filed

Necropsy Information

### Sacrifice Schedule: Group 1 An 1-10: Day 3

### Group 1 An 11-20: Day 5

### Group 1 An 21-30: Day 7

Method of Euthanasia: Bleed by cardiac puncture to exsanguinate followed by cervical dislocation.

### Time Points: Not Timed

[symbol]

| [symbol] | [symbol] | [symbol] | [symbol] | [symbol] |
|---|---|---|---|---|
| [symbol] t Injected | All | Remove skin and | Flash Freeze | Ship |
| Knee | | muscle keening | (15 ml conical | |
| | | joint | vial*) | |
| Left Injected | All | Remove skin and | Flash Freeze | Ship |
| Knee | | muscle keening | (15 ml conical | |
| | | joint | vial*) | |

*Label tubes with only mouse number, day of collection, and left or right leg. Samples will be tested without reference to whether they are AAV-2 or AAV-5 injected. Key to be provided only after PCR completion. [symbol]
indicates data missing or illegible when filed

Sample Analysis

Tissue Specimens—Hind limbs from AAV-injected mice were snap-frozen and shipped. On arrival, specimens were transferred to the −80° C. freezer for storage.

GFP Expression in Target Tissues—Hind limbs (paired) were thawed at room temperature and imaged in an IVIS bioluminescence imaging system (Lumina III; Perkin Elmer). GFP fluorescence was quantified using excitation at 488 nm and measuring emission at 510 nm. A total of 4 mice were evaluated at each time point (3 days, 5 days and 7 days). Tissues from the remaining 6 animals at each time point were retained for subsequent confirmation of viral burden using real-time PCR.

Results—As can be seen in FIG. 3, there was high-level expression of GFP within injected knee joints at 3 days post-injection. Viral loads decreased slightly at 5 days, then rose again to 7 days. With the limited sample size in this pilot study there was no significant difference between the behaviours of AAV-5 and AAV-6.

Discussion—The data from this study support the use of either AAV-5 or AAV-6 for intra-articular delivery of CRISPR-Cas9 into the mouse knee joint. The levels of both viral serotypes increased from 5 to 7 day, leaving open the possibility that they may have increased further if the follow-up had been extended to 2 or maybe 3 weeks. Additional work would be needed to confirm this, but the data thus far would suggest that there should be an interval of at least one week before the injection of the vector and challenge with intra-articular monoiodoacetate (MIA) crystals.

Background & Rationale—The monoiodoacetate (MIA)-induced OA model is used in this work for two reasons. First, natural (spontaneous) OA is extremely uncommon in mice, whereas the injection of MIA results in an induced model of OA that is relatively fast in onset, predictable and that provides good clinical correlation to the disease phenotype see in human OA patients, including intra-articular inflammation, pain and cartilage degeneration. Second, in contrast with surgical models such as destabilization of the medial meniscus (DMM) and transection of the anterior cruciate ligament (ACLT), the MIA model does not involve surgical incision of the joint capsule, making it much more relevant to the capsules of human patients with OA.

Injection of MIA crystals in rodents reproduces OA-like lesions and functional impairment that can be analyzed and quantified by techniques such as behavioral testing and objective lameness assessment. MIA is an inhibitor of glyceraldehyde-3-phosphatase and the resulting alterations in cellular glycolysis eventual cause the death of cells within the joint, including chondrocytes. Chondrocyte death manifests as cartilage degeneration and alterations in proteoglycan staining. Mice injected with MIA usually exhibit pain-like behavior within 72 hours, and cartilage loss by around 4 weeks post-injection. Increases in IL-1 expression have been documented within 2-3 days of injection in rats and in mice.

Study Design—Mice are injected unilaterally with either MIA or the saline vehicle control (one joint per animal). Within each group, half of the animals are pre-treated with the AAV-CRISPR-Cas9 vector targeting the mouse IL-1 beta gene, and the other half are injected with an AAV-CRISPR-Cas9 scrambled control. Animals from both groups will be taken off study at one of two time points: an early time point of 48 hours, to allow for assessment of the impact of therapy on the levels of IL-1 within the synovial fluid, and a late time point of 4 weeks to allow for assessment of the impact of therapy on cartilage breakdown and histological evidence of osteoarthritis.

Methods

Experimental Animals—A total of 80 mice are used in this study. The experimental procedures are reviewed and approved by the local IACUC. Mice are housed in micro-isolator cages, fed a standard laboratory animal diet, and allowed access to water ad libitum.

MIA Model & Anti-IL1 Therapy—Mice are acclimated for a period of 7 days ahead of the study. On the first day of the study, mice are anaesthetized with an inhaled mixture of isoflurane in oxygen. Once a surgical plane of anesthesia has been confirmed, the right hind limb is clipped and the skin scrubbed with a surgical antiseptic. 40 mice (Treated) receive an intra-articular injection of the AAV-CRISPR-Cas9 vector targeting IL-1, and the remaining 40 animals (Control) are injected intra-articularly with the AAV-CRISPR-Cas9 scrambled control. Seven days later, half of the animals in each group are injected in the same joint with MIA and half with the saline vehicle. This leads to the establishment of four study groups:

Group 1: Treated-MIA (20 mice)

Group 2: Control-MIA (20 mice)

Group 3: Treated-Vehicle (20 mice)

Group 4: Control-Vehicle (20 mice)

Ten mice per group are euthanised 48 hours after the MIA challenge in order to document IL-1 levels in the knee joint. The remaining animals will be housed for 4 weeks in order to evaluate the effects of therapy on pain behavior (behavioral testing, including von Frey testing), lameness (limb use), joint swelling (caliper measurement) and joint pathology (histopathology).

Euthanasia & Tissue Collection—Mice are killed by exsanguination, followed by cervical dislocation. Joints are opened and either flushed for IL-1 measurement (48-hour group) or immersion fixed in 10% formalin for decalcified histopathology (4-week group).

### Example 5. Efficacy of AAV-6 and AAV-5 Mediated CRISPR Treatment in MSU-Crystal Induced Joint Arthritis in Mice

Introduction and Objectives

The objective of these studies is to identify compounds/proteins that inhibit the inflammation induced by monosodium urate (MSU) crystal induced release of interleukin 1J3 (IL-1J3). This is a simple prescreen that identifies anti-inflammatory activity of various types of anti-inflammatory agents, especially IL-1 pathway blockers like interleukin receptor antagonists or antibodies that block IL-1 or IL1R1 (Torres R, et al. Hyperalgesia, synovitis and multiple biomarkers of inflammation are suppressed by interleukin 1 inhibition in a novel animal model of gouty arthritis. Ann Rheym Dis. 2009; 68(10):1602-1608, which is incorporated by reference herein in its entirety for all purposes). Gout is the most common form of inflammatory arthritis and is increasing in prevalence worldwide (Roddy E and Doherty M. Epidemiology of Gout. Arthritis Research & Therapy. 2010; 12(6):223, which is incorporated by reference herein in its entirety for all purposes). Gouty arthritis is characterized by increased serum urate concentration and deposits of monosodium urate crystals (MSU) in and around the joints, leading to swollen joints and severe pain (Sabina E P, Chandel S, and Rasool M K. Inhibition of monosodium urate crystal-induced inflammation by withaferin A. J Pharm Pharmaceut Sci. 2008; 11(4):46-55, which is incorporated by reference herein in its entirety for all purposes). Current treatments include nonsteroidal anti-inflammatory drugs (NSAIDs), steroids, or colchicine. For some patients these treatments may not be effective in treating gout or have adverse side effects (Sabina, 2008; Getting S J, et al. Activation of melanocortin type 3 receptor as a molecular mechanism for adrenocorticotropic hormone efficacy in gouty arthritis. Arthritis & Rheumatism. 2002; 46(10):2765-2775, which is incorporated by reference herein in its entirety for all purposes). The MSU-induced inflammation model provides a good, simple screening tool for identifying compounds that may have activity in the more complex disease process, such as systemic arthritis and more complex IL-1 driven diseases.

A study was conducted to evaluate the efficacy of adeno-associated virus (AAV)-mediated CRISPR therapy in monosodium urate (MSU) crystal induced inflammation in mice. On study day 0, male C57BL/6 mice were dosed into the right knee with a single (1×) intra-articular (IA) injection of placebo control (diluent, phosphate buffered saline [PBS]), a mixture of two variants of AAV-6 (one carrying Guide RNA 1 and the other carrying Guide RNA 2, 5×109 virus genome [vg] copies per mL), a mixture of two variants of AAV-5 (Guide 1+Guide 2, 5×109 vg/mL), the scrambled AAV-6 control (carrying non-targeting guide RNA, 1×1010 vg/mL), or the scrambled AAV-5 control (1×1010 vg/mL). On study day 7, the mice were given injections into right knee (same joint as treatment) with MSU crystals (25 mg/mL: 250 μg in 10 μL PBS). The mice were euthanized for necropsy approximately 6 hours post-MSU injection on study day 7. Efficacy evaluation was based on animal body weights, von Frey testing, and knee caliper measurements.

Mice treated IA (1× on day 0) with AAV-6 (Guide 1+2: 5×109 vg/guide per knee) showed a statistically significant reduction in referred pain, as measured by von Frey testing, 6 hours after MSU injection on day 7 as compared to mice injected IA with AAV-5 scramble vector (p=0.025) with results being nearly significant as compared to the AAV-6 scramble vector and PBS control groups (p=0.051 and p=0.075, respectively). Area under the curve (AUC) calculations for von Frey assessments did not differ statistically across groups. Animal body weight gain and knee swelling did not differ statistically across groups (Table 8). All animals survived to study termination.

TABLE 8
Summary of Data

| | | von Frey Absolute | Knee Caliper Change |
|---|---|---|---|
| | | Threshold AUC | from Baseline AUC |
| Group Treatment | | (day −1 to [symbol] | (day −1 to 7) − [symbol] |
| 1 | AAV-6 Scramble Vector (1 × 10¹⁰ | 5.92 (0.39) | 0.02 (0.01) |
| | particles/knee), IA, 1× (d0) | | |
| 2 | AAV-6 Guide 1 + 2 (5 × 10⁹ | 6.01 (0.36) | 0.01 (0.01) |
| | of each guide/knee), IA, 1× (d0) | | |
| 3 | PBS, IA (d0) | 6.12 (0.33) | 0.02 (0.01) |
| 4 | AAV-5 Scramble Vector (1 × 10¹⁰ | 5.09 (0.31) | 0.02 (0.01) |
| | particles/knee), IA, 1× (d0) | | |
| 5 | AAV-5 Guide 1 + 2 (5 × 10⁹ | 5 73 (0.39) | 0 02 (0.01) |
| | of each guide/knee), IA, 1× (d0) | | |

Values represent group means and standard errors (SE)
PBS = Phosphate buffered saline control,
AAV = adeno-associated virus,
AUC = Area under the curve
*p < 0.05 ANOVA (Tukey's post-hoc) vs. AAV-6 Guide 1
†p < 0.05 ANOVA (Tukey's post-hoc) vs. PBS
‡p < 0.05 ANOVA (Tukey's post-hoc) vs. AAV-5 Scramble Vector
§p < 0.05 ANOVA (Tukey's post-hoc) vs. AAV-5 Guide 1 [symbol]
indicates data missing or illegible when filed

Summary of Clinical Outcomes—No significant differences were observed between groups over time. No clinical evidence that virus injection provoked a response greater than that seen in the vehicle group. No clinical evidence that virus injection altered the effects of MSU on joint swelling. Specific role of IL-1 in MSU-induced inflammation is unclear, so lack of clinical effect may not be unexpected.

Summary of qPCR—qPCR data confirm that CRISPR editing with AAV-6 or AAV-5 is effective in restoring IL-1 beta mRNA expression to normal levels. Statistical significance is hard to demonstrate given the sample size. Confirmation of this effect can be obtained through IHC analysis of synovial tissues.

Regulatory Compliance

This study was conducted in accordance with the test facility standard operating procedures (SOPs), the World Health Organization Quality Practices in Basic Biomedical Research guidelines, and in compliance with all state and federal regulations, including USDA Animal Welfare Act 9 CFR Parts 1-3. Federal Register 39129, Jul. 22, 1993.

Institutional Animal Care and Use

This study was conducted in accordance with The Guide for the Care & Use of Laboratory Animals (8^{th} Edition). No acceptable alternative test systems were identified for the animals used in this study.

Materials and Methods

Test Article Identification and Preparation

AAV vectors were pre-formulated as a viral particle suspension (>5×10¹² virus genome [vg] copies per mL) in frozen aliquots. The aliquots were stored at −80° C. and reconstituted in diluent (sterile filtered PBS [Corning, lot No. 01420007]) immediately before use. Standard biosafety level 2 (BSL-2) handling was used by personnel handling the AAV vectors prior to injection. The AAV scramble controls were prepared in sterile PBS to form working stocks containing 1×10¹² vg/mL for IA injection at 10 μL/knee to deliver 1×10¹⁰ vg of the scramble control into the knee joint. The active AAV vectors were prepared by mixing equal parts of each of the two active AAV-5 or AAV-6 constructs with sterile PBS to form working stocks containing 5×10¹¹ vg/mL for each of the two guides. The active AAV formulations were injected IA at 10 μL/knee to deliver 5×10⁹ vg of each of the two guides into the knee joint. See the study protocol (Appendix B) for further details of test article preparation, storage, and handling.

The AAV vectors were identified as follows:

AAVPrimeTM Adeno-Associated Virus - Serotype6 (AAV-6)
Particles for sgRNA (GeneCopoeia ™, catalog
No. AA06-MCP001682-AD01-2-200-a, lot No. GC03182K2001)
AAVPrimeTM Adeno-Associated Virus - Serotype6 (AAV-6)
Particles for sgRNA (GeneCopoeia ™, catalog
No. AA06-MCP001682-AD01-2-200-b, lot No. GC03202K2001)
AAVPrimeTM Adeno-Associated Virus - Serotype6 (AAV-6)
Particles for sgRNA (GeneCopoeia ™, catalog
No. AA06-CCPCTR01-AD01-200, lot No. GC03112K2002)
AAVPrimeTM Adeno-Associated Virus - Serotype5 (AAV-5)
Particles for sgRNA (GeneCopoeia ™, catalogue
No. AA05-MCP001682-AD01-2-200A, lot No. GC03182K2002)
AAVPrimeTM Adeno-Associated Virus - Serotype5 (AAV-5)
Particles for sgRNA (GeneCopoeia ™, catalogue
No. AA05-MCP001682-AD01-2-200B, lot No. GC03182K2003)
AAVPrimeTM Adeno-Associated Virus - Serotype5 (AAV-5)
Particles for sgRNA (GeneCopoeiaTM, catalogue
No. AA05-CCPCTR01-AD01-200, lot No. GC03032K2003)

Monosodium urate (MSU) crystals were obtained from Invivogen (catalogue No. Tlrl-25-MSU, lot No. MSU-42-01). MSU crystals were prepared at 25 mg/mL in PBS (without Ca or Mg: Corning, catalogue No. 21-031-CV, lot No. 31719003) in a plastic tube, vortexed for approximately 1 minute, sonicated for approximately 15 to 20 minutes, and vortexed before pipetting and use.

Test System

| | |
|---|---|
| Number of animals: | 75 (70 + 5 extra) |
| Species/Strain or Breed: | C57BL/6 |
| Vendor: | Jackson |
| Age/Wt at Arrival: | 8-10 weeks old (~20 grams) |
| Gender: | Male |
| Age/Wt Range at Study Initiation: | At least 9 weeks by study initiation |
| Acclimation: | Will be acclimated for at least 3 |
| | days after arrival at BBP |
| Housing: | 3-5 animals/cage |

Male C57BL/6 mice (N=70+4 extra) that were 8 to 10 weeks of age were obtained from The Jackson Laboratory (Bar Harbor, Me.). The mice weighed approximately 20 to 29 grams (mean of approx. 25 g) at enrollment on study day −1.

Animals were identified by color-coded dots at the base of the tail delineating animal number. After enrollment, all cages were labeled with protocol number, group number, and animal numbers.

Environment and Husbandry

Upon arrival, the animals were housed 3 to 5 per cage in polycarbonate cages with corncob bedding and suspended food and water bottles. The mice were housed in individually ventilated pie cages (passive airflow, approximately 0.045-0.048 m2 floor space). Animal care including room, cage, and equipment sanitation conformed to the guidelines cited in the Guide for the Care and Use of Laboratory Animals (Guide, 2011) and the applicable BBP SOPs.

The animals were acclimated for 9 days prior to being paced in the study. An attending veterinarian was on site or on call during the live phase of the study. No concurrent medications were given.

During the acclimation and study periods, the animals were housed in a laboratory environment with temperatures ranging 19° C. to 25° C. and relative humidity of 30% to 70%. Automatic timers provided 12 hours of light and 12 hours of dark. The animals were allowed access ad libitum to Envigo Teklad 8640 diet fresh municipal tap water.

Study Design

On study day −1, the animals were randomized by body weight into treatment groups, knees were shaved, and baseline knee caliper measurements were taken. On study day 0, the animals were dosed with treatments (IA into the right knee) as indicated in Table 9. On study day 7, the animals were given IA injections of MSU crystals (a total of 10 μL, 250 μg of MSU) into right knees (same knee as treatments). Body weight measurements were taken as described. Referred pain was measured by von Frey testing at 5 time points as described. Caliper measurements of right knees were taken at 5 time points as described. The animals were euthanized for necropsy following the final behavioral testing on day 7, as described.

TABLE 9
Group and Treatment Information

| | | | | Dose Level | Dose | | Dose Conc. | | |
|---|---|---|---|---|---|---|---|---|---|
| Grp | N | MSU | Treatment | (particles/knee) | Vol. | | (particles/mL) | Dose Route | Regimen |
| 1 | 14 | Yes | AAV-6 | 1 × 10¹⁰ | 10 | μL | 1 × 10¹² | IA (right knee) | 1x (Day 0) |
| | | | Scramble | | | | | | |
| | | | Vector | | | | | | |
| 2 | 14 | Yes | AAV-6 | 5 × 10⁹ | 10 | μL | 1 × 10¹² | IA (right knee) | 1x (Day 0) |
| | | | Guide 1 + 2 | (of each | | | | | |
| | | | | guide) | | | | | |
| 3 | 14 | Yes | PBS | — | 10 | μL | — | IA (right knee) | 1x (Day 0) |
| 4 | 14 | Yes | AAV-5 | 1 × 10¹⁰ | 10 | μL | 1 × 10¹² | IA (right knee) | 1x (Day 0) |
| | | | Scramble | | | | | | |
| | | | Vector | | | | | | |
| 5 | 14 | Yes | AAV-5 | 5 × 10⁹ | 10 | μL | 1 × 10¹² | IA (right knee) | 1x (Day 0) |
| | | | Guide 1 + 2 | (of each | | | | | |
| | | | | guide) | | | | | |

Disease Induction

MSU crystals were prepared at a concentration of 25 mg/mL in sterile PBS. Crystals did solubilize, and injection preparation was carefully mixed prior to use. 10 uL of MSU crystal solution was injected into the right knee joint.

Unformulated MSU Storage Conditions: Room temperature, protected
from light
Vehicle: PBS
Dose Route: IA into the right knee
Dose Volume: 10 uL per knee
MSU Formulation Instructions & Calculations: 0.01 ml/knee * 75
animals = 0.72 ml of 25 mg/ml MSU needed, minimum
MSU Storage & Stability: Stored in glass. Prepared just prior to dosing.
Vortexed before injecting
MSU Disposition Following Dosing: Formulations discarded after dosing.
Unformulated stock retained.

Body Weight Measurements and Live Phase Sampling

The mice were weighed on study day −1 (pre-injection) for randomization, and body weights were measured again on study days 2 and 6. Animal body weight measurements can be found in Table 10.

TABLE 10
Body Weight and Dose Calculation Data (MGA-UCM-2)

| Treatment | | | | | | | |
|---|---|---|---|---|---|---|---|
| Group | | | | | | | |
| Group 1 | | | | | | | |
| AAV -6 | | | Day 2 | | Day 6 | | |
| Scramble Vector | | | | Body | | Body | Change in |
| (1 × 10\hat{ }10 | Day −1 | Day 0 | | Wt. %/ | | Wt. %/ | Body |
| particles/knee) | Body | Dose Vol. | Body | Δ from | Body | Δ from | Weight |
| IA, Ix (D 0) | Wt. (g) | 10 μl (ml) | Wt. (g) | Baseline | Wt. (g) | Baseline | Day −1 to 6 |
| 1 | 25.99 | 0.01 | 24.19 | −6.9% | 24.67 | [symbol] | −1 |
| 2 | 25.00 | 0.01 | 24.64 | −1.4% | 25.32 | 1.3% | 0 |
| 3 | 23.50 | 0.01 | 24.61 | 4.7% | [symbol] | [symbol] | 1 |
| 4 | 23.92 | 0.01 | [symbol] | [symbol] | 23.81 | −0.5% | 0 |
| 5 | 23.30 | 0.01 | [symbol] | −0.7% | 23.78 | 2.1% | 0 |
| 6 | 23.64 | 0.01 | [symbol] | 4.0% | 24.51 | [symbol] | 1 |
| 7 | 27.44 | 0.01 | 28.10 | 2.4% | 27.90 | 1.7% | 0 |
| 8 | 22.79 | 0.01 | 22.83 | 0.2% | 23.59 | 3.5% | 1 |
| 9 | 24.80 | 0.01 | 24.74 | −0.2% | 25.71 | 3.7% | 1 |
| 10 | 27.46 | 0.01 | 27.97 | 1.9% | 28.86 | 4.4% | 1 |
| 11 | 26.50 | 0.01 | 26.57 | 0.3% | 28.88 | 1.4% | 0 |
| 12 | 26.51 | 0.01 | 26.48 | −0.1% | 27.78 | 4.8% | 1 |
| 13 | 22.80 | 0.01 | 23.58 | 2.5% | 23.77 | 4.3% | 1 |
| 14 | 24.77 | 0.01 | 24.60 | −0.7% | 24.90 | 0.5% | 0 |
| Extra | 21.95 | 0.01 | 22.56 | 0.8% | 23.34 | 6.3% | 1 |
| Mean | 24.84 | | 24.88 | 0.19% | 25.4 | 2.47% | 0.6 |
| SE | 0.45 | | 0.5 | 0.80% | 0.5 | 0.80% | 0.2 |
| ANOVA | | | | | | | 0.683 |
| (Tukey's post-hoc) | | | | | | | |
| vs. Group 2 | | | | | | | |
| ANOVA | | | | | | | 0.243 |
| (Tukey's post-hoc) | | | | | | | |
| vs. Group 3 | | | | | | | |
| ANOVA | | | | | | | 0.213 |
| (Tukey's post-hoc) | | | | | | | |
| vs. Group 4 | | | | | | | |
| ANOVA | | | | | | | 0.543 |
| (Tukey's post-hoc) | | | | | | | |
| vs. Group 5 | | | | | | | |

| Group 3 | | | | | | | |
|---|---|---|---|---|---|---|---|
| AAV -6 | | | | | | | |
| Guide 1 + 2 | | | Day 2 | | Day 6 | | |
| (5 × 10\hat{ }9 of | Day −1 | Day −1 | | Body | | Body | Change in |
| each guide/knee) | Body | Dose Vol. | Body | Wt. %/Δ | Body | Wt. %/Δ | Body Weight |
| IA, Ix (D 0) | Wt. (g) | 10 μl (ml) | Wt. (g) | Baseline | Wt. (g) | Baseline | Day −1 to 6 |
| 1 | 22.36 | 0.01 | 22.47 | −0.4% | 22.42 | 1.6% | 0 |
| 2 | 28.49 | 0.01 | 24.00 | 1.8% | 29.08 | 2.1% | 1 |
| 3 | 24.65 | 0.01 | [symbol] | 0.0% | 25.06 | 1.7% | 0 |
| 4 | 23.63 | 0.01 | 23.29 | −0.4% | 24.03 | 1.8% | 0 |
| 5 | 22.78 | 0.01 | 23.19 | 1.8% | 24.14 | 6.0% | 1 |
| 6 | 27.32 | 0.01 | 27.60 | 1.0% | 28.54 | 4.5% | 1 |
| 7 | 26.83 | 0.01 | 27.49 | 2.6% | 28.00 | 4.4% | 1 |
| 8 | 26.86 | 0.01 | 26.84 | −0.4% | 28.70 | 6.5% | 2 |
| 9 | 22.80 | 0.01 | 22.88 | 0.4% | 23.66 | 3.8% | 1 |
| 10 | 22.45 | 0.01 | [symbol] | 3.0% | 23.86 | 6.3% | 1 |
| 11 | 24.83 | 0.01 | 24.53 | −1.2% | 25.42 | 2.4% | 1 |
| 12 | 25.80 | 0.01 | 26.11 | 1.2% | 26.56 | 2.9% | 1 |
| 13 | 25.10 | 0.01 | 25.59 | 1.1% | 28.14 | 3.3% | 1 |
| 14 | 23.45 | 0.01 | 23.88 | 1.8% | 24.14 | 2.9% | 1 |
| Extra | 25.84 | 0.01 | 26.43 | 2.3% | 26.29 | 1.3% | 0 |
| Mean | 24.85 | | 25.03 | 0.79% | 25.7 | 3.57% | 0.9 |
| SE | 0.53 | | 0.56 | 0.36% | 0.6 | 0.46% | 0.1 |
| ANOVA | | | | | | | 0.940 |
| (Tukey's pos-hoc) | | | | | | | |
| vs. Group 3 | | | | | | | |
| ANOVA | | | | | | | 0.019 |
| (Tukey's pos-hoc) | | | | | | | |
| vs. Group 4 | | | | | | | |
| ANOVA | | | | | | | 0.999 |
| (Tukey's pos-hoc) | | | | | | | |
| vs. Group 5 | | | | | | | |

| | | | Day 2 | | Day 6 | | |
|---|---|---|---|---|---|---|---|
| | Day −1 | Day −1 | | Body | | Body | Change in |
| Treatment | Body | Dose Vol. | Body | Wt. %/Δ | Body | Wt. %/Δ | Body Weight |
| Group | Wt. (g) | 10 μl (ml) | Wt. (g) | Baseline | Wt. (g) | Baseline | Day −1 to 6 |
| Group 3 | | | | | | | |
| PBS | | | | | | | |
| IA, Ix (D 0) | | | | | | | |
| 1 | [symbol] | 0.01 | [symbol] | 3.0% | [symbol] | 7.6% | 2 |
| 2 | [symbol] | 0.01 | [symbol] | [symbol] | [symbol] | [symbol] | 1 |
| 3 | 24.72 | 0.01 | [symbol] | [symbol] | [symbol] | [symbol] | 1 |
| 4 | [symbol] | 0.01 | [symbol] | 1.8% | [symbol] | [symbol] | 1 |
| 5 | 25.50 | 0.01 | [symbol] | 1.9% | [symbol] | [symbol] | 1 |
| 6 | 25.23 | 0.01 | [symbol] | 0.3% | [symbol] | 3.1% | 1 |
| 7 | [symbol] | 0.01 | [symbol] | [symbol] | [symbol] | [symbol] | 1 |
| 8 | [symbol] | 0.01 | [symbol] | 2.6% | 26.83 | 4.0% | 1 |
| 9 | [symbol] | 0.01 | [symbol] | 2.2% | [symbol] | [symbol] | 1 |
| 10 | [symbol] | 0.01 | [symbol] | 1.8% | 27.14 | [symbol] | 1 |
| 11 | [symbol] | 0.01 | [symbol] | [symbol] | [symbol] | [symbol] | 1 |
| 12 | [symbol] | 0.01 | [symbol] | 1.3% | [symbol] | [symbol] | 1 |
| 13 | [symbol] | 0.01 | 24.88 | [symbol] | [symbol] | [symbol] | 1 |
| 14 | [symbol] | 0.01 | [symbol] | [symbol] | 24.92 | [symbol] | 0 |
| Mean | [symbol] | | [symbol] | [symbol] | [symbol] | [symbol] | 1.1 |
| SE | [symbol] | | | 0.36% | 0.6 | [symbol] | 0.1 |
| ANOVA | | | | | | | [symbol] |
| (Tukey's pots-hoc) | | | | | | | |
| vs. Group 4 | | | | | | | |
| ANOVA | | | | | | | [symbol] |
| (Tukey's post-hoc) | | | | | | | |
| vs. Group 5 | | | | | | | |
| Group 4 | | | | | | | |
| AAV-4 | | | | | | | |
| Scramble Vector | | | | | | | |
| (1 × 10\hat{ }10 | | | | | | | |
| particles/knee) | | | | | | | |
| IA, Ix (D 0) | | | | | | | |
| 1 | [symbol] | [symbol] | 25.63 | [symbol] | [symbol] | [symbol] | 2 |
| 2 | [symbol] | [symbol] | [symbol] | [symbol] | 30.89 | [symbol] | 2 |
| 3 | [symbol] | [symbol] | [symbol] | [symbol] | [symbol] | [symbol] | 0 |
| 4 | [symbol] | [symbol] | 23.95 | [symbol] | [symbol] | [symbol] | 1 |
| 5 | [symbol] | [symbol] | [symbol] | [symbol] | [symbol] | [symbol] | 1 |
| 6 | [symbol] | [symbol] | [symbol] | 1.6% | 27.89 | 3.5% | 1 |
| 7 | [symbol] | [symbol] | 25.87 | 1.2% | [symbol] | [symbol] | 1 |
| 8 | [symbol] | [symbol] | [symbol] | [symbol] | [symbol] | [symbol] | 1 |
| 9 | [symbol] | [symbol] | [symbol] | [symbol] | 27.21 | 10.3% | 1 |
| 10 | [symbol] | [symbol] | [symbol] | [symbol] | [symbol] | 6.1% | 1 |
| 11 | [symbol] | [symbol] | [symbol] | [symbol] | [symbol] | 0.8% | 0 |
| 12 | [symbol] | [symbol] | [symbol] | [symbol] | [symbol] | 6.2% | 1 |
| 13 | [symbol] | [symbol] | [symbol] | −0.1% | [symbol] | 2.0% | 0 |
| 14 | [symbol] | [symbol] | [symbol] | [symbol] | [symbol] | 1.3% | 0 |
| Extra | [symbol] | [symbol] | [symbol] | [symbol] | [symbol] | [symbol] | 0 |
| Mean | [symbol] | | [symbol] | [symbol] | [symbol] | [symbol] | 1.1 |
| SE | 0.42 | | 8.47 | [symbol] | [symbol] | [symbol] | 0.2 |
| ANOVA | | | | | | | [symbol] |
| (Tukey's post-hoc) | | | | | | | |
| vs. Group 5 | | | | | | | |
| Group 5 | | | | | | | |
| [symbol] | | | | | | | |
| Guide 1 + 2 | | | | | | | |
| (5 × 10\hat{ }9 of | | | | | | | |
| each guide/knee) | | | | | | | |
| IA, Ix (D 0) | | | | | | | |
| 1 | [symbol] | [symbol] | [symbol] | [symbol] | [symbol] | [symbol] | 1 |
| 2 | [symbol] | [symbol] | [symbol] | [symbol] | 24.18 | 2.1% | 1 |
| 3 | [symbol] | [symbol] | 24.42 | [symbol] | 24.71 | 1.8% | 0 |
| 4 | [symbol] | [symbol] | [symbol] | 1.0% | [symbol] | [symbol] | 1 |
| 5 | [symbol] | [symbol] | [symbol] | 3.3% | 24.97 | [symbol] | 0 |
| 6 | [symbol] | [symbol] | [symbol] | [symbol] | 27.75 | [symbol] | 1 |
| 7 | 25.92 | [symbol] | [symbol] | [symbol] | [symbol] | [symbol] | 0 |
| 8 | 25.32 | [symbol] | 23.48 | [symbol] | [symbol] | [symbol] | 1 |
| 9 | [symbol] | [symbol] | 27.51 | [symbol] | [symbol] | [symbol] | 1 |
| 10 | 27.50 | [symbol] | 27.63 | [symbol] | [symbol] | [symbol] | 1 |
| 11 | [symbol] | [symbol] | [symbol] | [symbol] | [symbol] | [symbol] | 1 |
| 12 | [symbol] | [symbol] | [symbol] | [symbol] | [symbol] | [symbol] | 1 |
| 13 | [symbol] | [symbol] | [symbol] | [symbol] | 34.95 | [symbol] | 0 |
| 14 | [symbol] | [symbol] | [symbol] | [symbol] | 36.22 | [symbol] | 1 |
| Extra | [symbol] | [symbol] | [symbol] | [symbol] | [symbol] | [symbol] | 1 |
| Mean | [symbol] | | 25.29 | [symbol] | [symbol] | [symbol] | 0.9 |
| SE | [symbol] | | [symbol] | [symbol] | 0.4 | [symbol] | 0.1 |

[symbol]

indicates data missing or illegible when filed

Von Frey Methods

Von Frey analysis was performed on right hind paws at 5 time points: baseline (day −1), 6 hours post-dose (day 0), 24 hours post-dose (day 1), pre-MSU injection (0 h, day 7), and 6 hours post-MSU injection (day 7). The groups were blinded to the researcher during von Frey testing.

The von Frey method evaluates mechanical allodynia (pain due to a stimulus that does not normally provoke pain) based on the response of animals to the application of calibrated filaments (Bioseb, Vitrolles, France) to the foot. The filaments are identified by a number representing log 10 of the force in milligrams×10. The animals are habituated to the testing rack three times (45 to 60 minutes) prior to baseline evaluation. When testing, the von Frey hair is placed on the surface of the hind paw and pushed smoothly until the hair has a significant bend in it; the hair is pressed against the paw for six seconds. Responses are recoded as either a 0 (no response) or a 1 (response). A response is defined as lifting the hind paw away from the hair, jerking the leg away, walking away from the hair, etc. The starting hair is 3.22, if the animal responds the tester moves down to 2.83, if there is no response to the 3.22 hair then the tester moves up to 3.61; the tester continues to test hairs based on the response and moves up or down, as appropriate. The hair increments are as follows: 1.65, 2.36, 2.44, 2.83, 3.22, 3.61, 3.84, 4.08, 4.17. Each paw is tested 5 times, moving up and down between hairs until the final filament is reached. Data is entered into a spreadsheet and used to translate the response rate into a paw withdraw threshold. Results of testing are converted to an absolute threshold (50% response rate) in grams, using the formula 10(x+yz)/10000, where x equals the log unit value of the final tested filament, y equals the tabular value for the response pattern from Dixon's up-and-down method for small samples (Dixon, 1965), and z equals the average interval between filament values. Testing is done on the hind portions of the hind paw as the heel tends to give a more reliable and sensitive response. The testers monitor the animals for hyper-responding or freezing, in which case the animals are left alone until calm. Von Frey data can be found in Table 11.

TABLE 11
von Frey Data (MGA-UCM-2)

| | Day −1 | | Day 0.25 | | Day 1 | | Day 7 | | Day 7.25 | | |
|---|---|---|---|---|---|---|---|---|---|---|---|
| | Absolute | | Absolute | | Absolute | | Absolute | | Absolute | | |
| | Threshold | | Threshold | | Threshold | | Threshold | | Threshold | | |
| Treatment | Log | | Log | | Log | | Log | | Log | | Day −1-7 |
| Group | units | (g) | units | (g) | units | (g) | units | (g) | units | (g) | AUC |
| Group 1 | | | | | | | | | | | |
| AAV -6 | | | | | | | | | | | |
| Scramble Vector | | | | | | | | | | | |
| (1 × 10\hat{ }10 | | | | | | | | | | | |
| particles/knee) | | | | | | | | | | | |
| IA, Ix (D 0) | | | | | | | | | | | |
| 1 | 3.72500 | 0.53 | 3.96000 | 0.93 | 3.96000 | 0.91 | 3.72500 | 0.53 | 3.72500 | 0.53 | 6.05 |
| 2 | 3.96000 | 0.91 | 3.72500 | 0.53 | 3.72500 | 0.53 | 4.12500 | 1.33 | 3.41500 | 0.26 | 7.09 |
| 3 | 3.72500 | 0.53 | 3.02300 | 0.11 | 3.02500 | 0.11 | 3.96000 | 0.91 | 3.41500 | 0.26 | 3.68 |
| 4 | [symbol] | 0.91 | 3.96000 | 0.91 | 3.96000 | 0.91 | 3.96000 | 0.91 | 2.63500 | 0.04 | 7.42 |
| 5 | 3.72500 | 0.53 | 3.72500 | 0.53 | [symbol] | 0.11 | 3.72500 | 0.53 | 3.41500 | 0.26 | 2.91 |
| 6 | 3.96000 | 0.91 | 3.72500 | 0.53 | 3.96000 | 0.91 | 3.96000 | 0.91 | 3.02500 | 0.11 | 7.04 |
| 7 | 4.12500 | 1.33 | 3.72500 | 0.53 | 3.41500 | 0.26 | 3.72500 | 0.53 | 3.41500 | 0.26 | 3.93 |
| 8 | 3.96000 | 0.91 | 3.96000 | 0.91 | 3.96000 | 0.91 | 3.96000 | 0.91 | 3.41500 | 0.26 | 7.44 |
| 9 | 3.72500 | 0.53 | 3.72500 | 0.53 | 3.96000 | 0.91 | 3.72500 | 0.53 | 3.41500 | 0.26 | 3.63 |
| 10 | 3.96000 | 0.91 | 3.96000 | [symbol] | 3.96000 | 0.91 | 3.96000 | 0.91 | 2.63500 | 0.04 | 7.42 |
| 11 | 3.96000 | 0.91 | 3.96000 | 0.91 | 3.72500 | 0.53 | 3.96000 | 0.91 | [symbol] | 0.11 | 6.14 |
| 12 | 4.12500 | 1.33 | 3.72500 | 0.53 | 3.72500 | 0.53 | 3.96000 | 0.91 | 2.63500 | 0.26 | 6.04 |
| 13 | 3.96000 | 0.91 | 3.72500 | 0.53 | 3.72500 | 0.53 | 3.96000 | 0.91 | 2.41500 | 0.04 | 5.75 |
| 14 | 3.96000 | 0.91 | [symbol] | [symbol] | 3.96000 | 0.91 | 3.72500 | 0.53 | 2.63500 | 0.53 | 6.29 |
| Extra | 4.12500 | 1.33 | 3.41500 | 0.26 | 3.41500 | 0.26 | 4.12500 | 1.33 | 3.02500 | 0.11 | 6.15 |
| Mean | | 0.86 | | 0.66 | | 0.64 | | 0.81 | | 0.23 | 5.92 |
| SE | | 0.07 | | [symbol] | | 0.08 | | 0.06 | | 0.04 | 0.39 |
| ANOVA | | 0.979 | | 0.917 | | 1.800 | | 0.974 | | [symbol] | 1.800 |
| (Tukey's post-hoc) | | | | | | | | | | | |
| vs Group 2 | | | | | | | | | | | |
| ANOVA | | 0.998 | | [symbol] | | >0.9999 | | 0.987 | | >0.9999 | 0.994 |
| (Tukey's post-hoc) | | | | | | | | | | | |
| vs. Group 3 | | | | | | | | | | | |
| ANOVA | | 0.999 | | 0.943 | | 0.493 | | 0.956 | | 0.987 | 0.478 |
| (Tukey's post-hoc) | | | | | | | | | | | |
| vs. Group 4 | | | | | | | | | | | |
| ANOVA | | 0.984 | | 0.873 | | 0.995 | | 0.998 | | 0.399 | 0.996 |
| (Tukey's post-hoc) | | | | | | | | | | | |
| vs. Group 5 | | | | | | | | | | | |
| Group 2 | | | | | | | | | | | |
| [symbol] | | | | | | | | | | | |
| Guide 1 + 2 | | | | | | | | | | | |
| (5 × 10\hat{ }9 of | | | | | | | | | | | |
| each guide/knee) | | | | | | | | | | | |
| IA, Ix (D 0) | | | | | | | | | | | |
| 1 | 3.96000 | 0.91 | 3.41500 | 0.26 | 3.72500 | 0.53 | 4.12500 | 1.33 | 3.41500 | 0.26 | 6.82 |
| 2 | 3.96000 | 0.91 | 3.02500 | 0.11 | 3.96000 | 0.91 | 3.72500 | 0.53 | 3.41500 | 0.26 | 5.45 |
| 3 | 3.72500 | 0.53 | 3.72500 | 0.53 | 3.41500 | 0.26 | 3.96000 | 0.91 | 3.72500 | 0.53 | 4.66 |
| 4 | 3.72500 | 0.53 | 3.96000 | 0.91 | 3.96000 | 0.91 | 3.96000 | 0.91 | 3.41500 | 0.26 | 7.20 |
| 5 | 3.96000 | 0.91 | 3.96000 | 0.91 | 3.96000 | 0.91 | 3.72500 | 0.53 | 3.72500 | 0.53 | 6.29 |
| 6 | 3.96000 | 0.91 | 3.96000 | 0.91 | 3.96000 | 0.91 | 4.32500 | 1.33 | 3.72500 | 0.53 | 8.79 |
| 7 | 4.12500 | 1.33 | 3.32500 | 0.53 | 3.72500 | 0.53 | 3.96000 | 0.91 | 3.96000 | 0.91 | 6.12 |
| 8 | 3.72500 | 0.53 | 3.41500 | 0.26 | [symbol] | 0.11 | 3.96000 | 0.91 | 3.96000 | 0.91 | 3.91 |
| 9 | 4.12500 | 1.33 | 3.41500 | 0.26 | 3.96000 | 0.91 | 3.72500 | 0.53 | 3.72500 | 0.53 | [symbol] |
| 10 | 3.96000 | 0.91 | 3.96000 | 0.91 | [symbol] | 0.11 | 3.96000 | 0.91 | 3.41500 | 0.26 | 4.72 |
| 11 | 3.96000 | 0.91 | 3.72500 | 0.53 | 3.72500 | 0.53 | 3.72500 | 0.53 | 3.72500 | 0.53 | 4.62 |
| 12 | 3.96000 | 0.91 | 3.96000 | 0.91 | 3.41500 | 0.26 | 3.96000 | 0.91 | 3.41500 | 0.26 | 5.24 |
| 13 | 3.96000 | 0.91 | 3.72500 | 0.53 | 3.96000 | 0.91 | 3.96000 | 0.91 | [symbol] | 0.11 | 7.04 |
| 14 | 4.12500 | 1.33 | 3.72500 | 0.53 | 3.96000 | 0.91 | 3.96000 | 0.91 | 3.72500 | 0.53 | [symbol] |
| Extra | 3.72500 | 0.53 | [symbol] | 0.91 | [symbol] | 0.53 | 3.72500 | 0.53 | [symbol] | 0.26 | 4.73 |
| Mean | | 0.92 | | 0.58 | | 0.62 | | 0.86 | | 0.46 | 6.01 |
| SE | | 0.07 | | 0.08 | | 0.09 | | 0.07 | | 0.06 | 0.36 |
| ANOVA | | 0.886 | | 0.941 | | >0.9999 | | 0.998 | | 0.075 | 1.000 |
| (Tukey's post-hoc) | | | | | | | | | | | |
| vs. Group 3 | | | | | | | | | | | |
| ANOVA | | 0.924 | | >0.9999 | | 0.639 | | 0.698 | | [symbol] | 0.369 |
| (Tukey's post-hoc) | | | | | | | | | | | |
| vs. Group 4 | | | | | | | | | | | |
| ANOVA | | 0.828 | | 1.000 | | 0.999 | | 0.999 | | 0.835 | 0.981 |
| (Tukey's post-hoc) | | | | | | | | | | | |
| vs. Group 5 | | | | | | | | | | | |
| Group 3 | | | | | | | | | | | |
| PBS | | | | | | | | | | | |
| IA, Ix (D 0) | | | | | | | | | | | |
| 1 | 3.96000 | 0.91 | 3.41500 | 0.26 | 3.96000 | 0.91 | 4.12500 | 1.33 | 3.72500 | 0.53 | 8.14 |
| 2 | 3.96000 | 0.91 | 3.96000 | 0.91 | 3.96000 | 0.91 | 3.96000 | 0.91 | 2.63500 | 0.04 | 7.42 |
| 3 | 3.96000 | 0.91 | 3.72500 | 0.53 | 3.96000 | 0.91 | 3.96000 | 0.91 | 3.41500 | 0.26 | 7.06 |
| 4 | 3.72500 | 0.53 | 3.96000 | 0.91 | 3.96000 | 0.91 | 3.72500 | 0.53 | 2.63500 | 0.04 | [symbol] |
| 5 | 3.72500 | 0.53 | 3.96000 | 0.91 | 3.72500 | 0.53 | 3.96000 | 0.91 | 2.63500 | 0.04 | 5.89 |
| 6 | 3.96000 | 0.91 | 3.96000 | 0.91 | 3.96000 | 0.91 | 3.72500 | 0.53 | 3.41500 | 0.26 | 6.25 |
| 7 | 3.96000 | 0.91 | 3.72500 | 0.53 | 3.72500 | 0.53 | 3.96000 | 0.91 | 3.72500 | 0.53 | 5.81 |
| 8 | 4.12500 | 1.33 | 3.72500 | 0.53 | 3.41500 | 0.26 | 3.72500 | 0.53 | 3.41500 | 0.26 | [symbol] |
| 9 | 3.72500 | 0.53 | 3.41500 | 0.26 | 3.72500 | 0.53 | 3.96000 | 0.91 | 3.41500 | 0.26 | 5.27 |
| 10 | 3.96000 | 0.91 | 3.96000 | 0.91 | 3.72500 | 0.53 | 3.96000 | 0.91 | 3.72500 | 0.53 | 6.19 |
| 11 | 3.72500 | 0.53 | 3.72500 | 0.53 | 3.72500 | 0.53 | 3.72500 | 0.53 | 2.63500 | 0.04 | [symbol] |
| 12 | 3.96000 | 0.91 | 3.96000 | 0.91 | 3.72500 | 0.53 | 4.12500 | 1.33 | 3.02500 | 0.11 | 7.45 |
| 13 | 3.96000 | 0.91 | 3.72500 | 0.53 | 3.72500 | 0.53 | 4.12500 | 1.33 | 3.02500 | 0.11 | 7.07 |
| 14 | 3.96000 | 0.91 | 3.72500 | 0.53 | 3.41500 | 0.26 | 3.96000 | 0.91 | 3.41500 | 0.26 | 4.86 |
| Mean | | 0.83 | | 0.66 | | 0.63 | | 0.89 | | [symbol] | 6.12 |
| SE | | 0.06 | | 0.87 | | 0.06 | | [symbol] | | 0.05 | [symbol] |
| ANOVA | | >0.9999 | | 0.962 | | [symbol] | | 0.548 | | 0.985 | 0.259 |
| (Tukey's post-hoc) | | | | | | | | | | | |
| vs. Group 4 | | | | | | | | | | | |
| ANOVA | | 0.999 | | 0.901 | | [symbol] | | 0.983 | | [symbol] | 0.937 |
| (Tukey's post-hoc) | | | | | | | | | | | |
| vs. Group 5 | | | | | | | | | | | |
| Group 4 | | | | | | | | | | | |
| AAV-4 | | | | | | | | | | | |
| Scramble Vector | | | | | | | | | | | |
| (1 × 10\hat{ }10 | | | | | | | | | | | |
| particles/knee) | | | | | | | | | | | |
| IA, Ix (D 0) | | | | | | | | | | | |
| 1 | 3.72500 | 0.53 | 3.96000 | 0.91 | 3.72500 | 0.53 | 3.96000 | 0.91 | [symbol] | 0.26 | 5.92 |
| 2 | 3.96000 | 0.91 | 3.72500 | 0.53 | 3.41500 | 0.26 | 3.72500 | 0.53 | 2.63500 | 0.04 | 3.64 |
| 3 | 3.72500 | 0.53 | 3.72500 | 0.11 | 3.72500 | 0.53 | 3.96000 | 0.91 | 3.41500 | 0.26 | 5.11 |
| 4 | 3.72500 | 0.53 | 3.72500 | 0.53 | 3.72500 | 0.53 | 3.72500 | 0.53 | 2.63500 | 0.04 | 4.32 |
| 5 | 4.12500 | 1.33 | 3.96000 | 0.91 | 3.72500 | 0.53 | 3.96000 | 0.91 | 3.41500 | 0.26 | 6.42 |
| 6 | 3.96000 | 0.91 | 3.96000 | 0.91 | 3.72500 | 0.53 | 3.72500 | 0.53 | 3.41500 | 0.26 | 4.97 |
| 7 | 3.96000 | 0.91 | 3.96000 | 0.91 | 3.72500 | 0.53 | 3.96000 | 0.91 | 2.63500 | 0.04 | 6.13 |
| 8 | 3.96000 | 0.91 | 3.96000 | 0.91 | [symbol] | 0.11 | 3.72500 | 0.53 | 3.41500 | 0.11 | 3.51 |
| 9 | 3.96000 | 0.91 | 3.72500 | 0.53 | 3.96000 | 0.91 | 3.96000 | 0.91 | 2.63500 | 0.04 | 7.03 |
| 10 | 3.96000 | 0.91 | 3.72500 | 0.53 | 3.02500 | 0.11 | 3.72500 | 0.53 | [symbol] | 0.26 | 3.15 |
| 11 | 4.12500 | 1.33 | 3.02500 | 0.11 | 3.72500 | 0.53 | 3.96000 | 0.91 | 3.72500 | 0.53 | 5.65 |
| 12 | 3.72500 | 0.53 | 3.41500 | 0.26 | 3.72500 | 0.53 | 3.96000 | 0.91 | 3.02500 | 0.11 | 5.25 |
| 13 | 3.72500 | 0.53 | 3.72500 | 0.53 | 3.72500 | 0.53 | 3.72500 | 0.53 | 3.72500 | 0.53 | 4.38 |
| 14 | 3.96000 | 0.91 | 3.72500 | 0.53 | 3.72500 | 0.53 | 3.96000 | 0.91 | 2.63500 | 0.04 | 5.75 |
| Extra | 3.96000 | 0.91 | 3.96000 | 0.91 | 3.72500 | 0.53 | 3.32500 | 0.53 | 3.41500 | 0.26 | [symbol] |
| Mean | | 0.84 | | 0.59 | | 0.48 | | 0.75 | | 0.20 | 5.09 |
| SE | | 0.07 | | 0.08 | | 0.05 | | 0.05 | | 0.05 | [symbol] |
| ANOVA | | 0.999 | | 0.999 | | 0.776 | | 0.869 | | 0.411 | 0.713 |
| (Tukey's post-hoc) | | | | | | | | | | | |
| vs. Group 5 | | | | | | | | | | | |
| Group 5 | | | | | | | | | | | |
| AAV -5 Guite 1 + 2 | | | | | | | | | | | |
| (5 × 10\hat{ }9 of | | | | | | | | | | | |
| each guide/knee) | | | | | | | | | | | |
| IA, Ix (D 0) | | | | | | | | | | | |
| 1 | 3.72500 | 0.53 | 3.96000 | 0.91 | 3.96000 | 0.91 | 3.96000 | 0.91 | [symbol] | 0.26 | 7.20 |
| 2 | 3.72500 | 0.53 | 3.41500 | 0.26 | 3.96000 | 0.91 | 3.96000 | 0.91 | 3.72500 | 0.53 | 6.59 |
| 3 | 4.12500 | 1.33 | 3.96000 | 0.91 | 3.72500 | 0.53 | 3.96000 | 0.91 | 3.96000 | 0.91 | 6.30 |
| 4 | 3.72500 | 0.53 | 3.02500 | 0.11 | 3.96000 | 0.91 | 3.72500 | 0.53 | 3.41500 | 0.26 | 5.21 |
| 5 | 3.72500 | 0.53 | 3.96000 | 0.91 | 3.96000 | 0.91 | 4.12500 | 1.33 | 3.96000 | 0.91 | [symbol] |
| 6 | 3.96000 | 0.91 | 3.96000 | 0.91 | 3.02500 | 0.11 | 3.72500 | 0.53 | 2.63500 | 0.04 | 3.50 |
| 7 | 3.72500 | 0.53 | 3.72500 | 0.53 | 3.72500 | [symbol] | 4.12500 | 1.33 | 3.02500 | 0.11 | 6.83 |
| 8 | 3.96000 | 0.91 | 3.41500 | 0.26 | [symbol] | 0.26 | 3.96000 | 0.91 | 3.72500 | 0.53 | 4.62 |
| 9 | 3.96000 | 0.91 | 3.72500 | 0.53 | 3.96000 | 0.91 | 3.96000 | 0.91 | [symbol] | 0.26 | 7.06 |
| 10 | 3.96000 | 0.91 | 3.02500 | 0.11 | 3.72500 | 0.53 | 3.72500 | 0.53 | 3.41500 | 0.26 | 4.16 |
| 11 | [symbol] | 0.53 | 3.96000 | 0.91 | [symbol] | 0.11 | 3.96000 | 0.91 | [symbol] | 0.26 | 4.48 |
| 12 | 3.96000 | 0.91 | 3.41500 | 0.26 | 3.96000 | 0.91 | 3.72500 | 0.53 | [symbol] | 0.04 | 3.57 |
| 13 | 4.12500 | 1.33 | 3.96000 | 0.91 | 3.41500 | 0.26 | 3.96000 | 0.91 | [symbol] | 0.53 | 5.34 |
| 14 | 3.96000 | 0.91 | 3.41500 | 0.26 | [symbol] | 0.53 | 3.72500 | 0.53 | [symbol] | 0.11 | 4.29 |
| Extra | 3.96000 | 0.91 | 3.02500 | 0.91 | 3.72500 | 0.53 | 3.96000 | 0.91 | [symbol] | 0.11 | 5.33 |
| Mean | | [symbol] | | 0.36 | | [symbol] | | 0.84 | | [symbol] | 5.73 |
| SE | | [symbol] | | [symbol] | | 0.09 | | 0.07 | | [symbol] | 0.39 |

[symbol]

indicates data missing or illegible when filed

Caliper Methods

Caliper measurements of right knees were taken at 5 time points: baseline (day −1), 6 hours post-dose (day 0), 24 hours post-dose (day 1), pre-MSU injection (0 h, day 7), and 6 hours post-MSU injection (day 7). Knee caliper measurements were made using a spring-loaded micrometer caliper (Mitutuyo). Knee caliper measurements can be found in Table 12.

TABLE 12
Knee Caliper Data (MGA-UCM-2)

| | Day −1 | | Day 0.25 | | Day 1 | | Day 7 | | Day 7.25 | | |
|---|---|---|---|---|---|---|---|---|---|---|---|
| | Caliper | | Caliper | | Caliper | | Caliper | | Caliper | | |
| | Measurement (mm) | | Measurement (mm) | | Measurement (mm) | | Measurement (mm) | | Measurement (mm) | | Day −1-7 |
| | | Δ from | | Δ from | | Δ from | | Δ from | | Δ from | Δ from |
| Treatment | | Baseline | | Baseline | | Baseline | | Baseline | | Baseline | Baseline |
| Group | Right | (D −1) | Right | (D −1) | Right | (D −1) | Right | (D −1) | Right | (D −1) | AUC |
| Group 1 | | | | | | | | | | | |
| AAV -6 | | | | | | | | | | | |
| Scramble Vector | | | | | | | | | | | |
| (1 × 10\hat{ }10 | | | | | | | | | | | |
| particles/knee) | | | | | | | | | | | |
| IA, Ix (D 0) | | | | | | | | | | | |
| 1 | 3.63 | 0.00 | 3.65 | 0.02 | 3.63 | 0.00 | 3.63 | 0.00 | 3.68 | 0.05 | 0.03 |
| 2 | [symbol] | 0.00 | 3.92 | 0.04 | 3.88 | 0.00 | 3.85 | −0.03 | 3.90 | 0.02 | −0.05 |
| 3 | 3.67 | 0.00 | 3.72 | 0.05 | 3.67 | 0.00 | [symbol] | [symbol] | [symbol] | 0.03 | 0.05 |
| 4 | 3.65 | 0.00 | 3.70 | 0.05 | 3.65 | 0.00 | 3.84 | −0.03 | [symbol] | 0.03 | 0.02 |
| 5 | 3.58 | 0.00 | 3.62 | 0.04 | [symbol] | 0.00 | 3.58 | [symbol] | [symbol] | 0.04 | 0.05 |
| 6 | 3.60 | 0.00 | 3.61 | 0.01 | [symbol] | 0.00 | [symbol] | [symbol] | [symbol] | 0.02 | 0.01 |
| 7 | [symbol] | 0.00 | 3.94 | 0.05 | [symbol] | 0.00 | [symbol] | −0.02 | 3.91 | 0.02 | −0.01 |
| 8 | 3.73 | 0.00 | 3.74 | 0.01 | 3.73 | 0.00 | 3.73 | [symbol] | 3.78 | 0.05 | 0.02 |
| 9 | [symbol] | 0.00 | 3.73 | 0.03 | 3.70 | 0.00 | [symbol] | −0.01 | 3.74 | 0.04 | [symbol] |
| 10 | 3.66 | 0.00 | [symbol] | 0.03 | 3.66 | 0.00 | [symbol] | 0.01 | 3.75 | [symbol] | 0.07 |
| 11 | 3.65 | 0.00 | [symbol] | 0.04 | [symbol] | 0.00 | [symbol] | [symbol] | [symbol] | 0.05 | 0.05 |
| 12 | 3.63 | 0.00 | [symbol] | 0.05 | [symbol] | 0.00 | [symbol] | [symbol] | 3.67 | 0.04 | 0.06 |
| 13 | 3.72 | 0.00 | 3.72 | [symbol] | 3.72 | 0.00 | 3.72 | [symbol] | 3.75 | 0.03 | [symbol] |
| 14 | 3.78 | 0.00 | 3.80 | 0.02 | 3.78 | 0.00 | 3.76 | −0.02 | 3.81 | 0.03 | −0.04 |
| Extra | [symbol] | 0.00 | [symbol] | 0.03 | 3.34 | 0.00 | 3.34 | 0.00 | [symbol] | 0.02 | 0.03 |
| Mean | 3.70 | 0.00 | 3.73 | 0.03 | 3.70 | 0.00 | 3.69 | −0.01 | 3.74 | 0.04 | 0.02 |
| SE | 0.03 | 0.00 | 0.03 | 0.08 | [symbol] | 0.00 | 0.02 | [symbol] | 0.02 | 0.08 | 0.01 |
| ANOVA | | | | 0.129 | | | | [symbol] | | 0.927 | 0.498 |
| (Tukey's post-hoc) | | | | | | | | | | | |
| vs. Group 2 | | | | | | | | | | | |
| ANOVA | | | | [symbol] | | | | [symbol] | | 0.543 | [symbol] |
| (Tukey's post-hoc) | | | | | | | | | | | |
| vs. Group 3 | | | | | | | | | | | |
| ANOVA | | | | [symbol] | | | | [symbol] | | [symbol] | 0.999 |
| (Tukey's post-hoc) | | | | | | | | | | | |
| vs. Group 4 | | | | | | | | | | | |
| ANOVA | | | | 0.370 | | | | 0.618 | | [symbol] | [symbol] |
| (Tukey's post-hoc) | | | | | | | | | | | |
| vs. Group 5 | | | | | | | | | | | |
| Group 2 | | | | | | | | | | | |
| AAV-6 | | | | | | | | | | | |
| Guide 1 + 2 | | | | | | | | | | | |
| (5 × 10\hat{ }9 of | | | | | | | | | | | |
| each guide/knee) | | | | | | | | | | | |
| IA, Ix (D 0) | | | | | | | | | | | |
| 1 | 3.48 | [symbol] | 3.48 | 0.00 | 3.48 | 0.00 | 3.48 | 0.00 | 3.51 | 0.03 | [symbol] |
| 2 | [symbol] | [symbol] | 4.04 | 0.00 | 4.04 | 0.00 | 4.02 | −0.02 | 4.04 | [symbol] | [symbol] |
| 3 | [symbol] | [symbol] | 3.67 | 0.00 | 3.67 | 0.00 | 3.67 | 0.00 | 3.71 | 0.04 | 0.01 |
| 4 | [symbol] | [symbol] | 3.62 | 0.00 | 3.62 | 0.00 | 3.62 | 0.00 | 3.67 | 0.05 | 0.01 |
| 5 | 3.58 | [symbol] | [symbol] | 0.00 | [symbol] | 0.00 | [symbol] | 0.00 | [symbol] | 0.04 | 0.01 |
| 6 | [symbol] | [symbol] | [symbol] | [symbol] | [symbol] | 0.00 | [symbol] | 0.01 | [symbol] | 0.04 | [symbol] |
| 7 | [symbol] | [symbol] | [symbol] | 0.01 | [symbol] | 0.00 | [symbol] | 0.00 | [symbol] | 0.02 | 0.01 |
| 8 | 3.73 | [symbol] | 3.75 | [symbol] | 3.75 | 0.00 | 3.75 | 0.00 | [symbol] | 0.04 | 0.01 |
| 9 | 3.38 | [symbol] | [symbol] | 0.04 | [symbol] | 0.00 | [symbol] | 0.00 | [symbol] | [symbol] | 0.05 |
| 10 | 3.64 | [symbol] | 3.64 | [symbol] | [symbol] | 0.00 | 3.64 | [symbol] | 3.67 | [symbol] | [symbol] |
| 11 | 3.65 | [symbol] | 3.70 | [symbol] | [symbol] | 0.00 | 3.65 | 0.00 | [symbol] | [symbol] | [symbol] |
| 12 | [symbol] | [symbol] | 3.75 | [symbol] | 3.75 | 0.00 | 3.74 | −0.01 | [symbol] | 0.03 | −0.03 |
| 13 | [symbol] | [symbol] | 3.68 | [symbol] | 3.68 | 0.00 | 3.68 | 0.00 | 3.68 | [symbol] | [symbol] |
| 14 | [symbol] | [symbol] | 3.69 | 0.04 | [symbol] | 0.00 | [symbol] | 0.00 | [symbol] | [symbol] | 0.04 |
| Extra | [symbol] | [symbol] | [symbol] | 0.02 | [symbol] | 0.00 | 3.82 | [symbol] | [symbol] | [symbol] | [symbol] |
| Mean | 3.70 | [symbol] | 3.71 | 0.01 | 3.78 | [symbol] | 3.78 | [symbol] | 3.73 | 0.03 | [symbol] |
| SE | [symbol] | [symbol] | 0.04 | 0.01 | 0.04 | [symbol] | [symbol] | [symbol] | [symbol] | [symbol] | [symbol] |
| ANOVA | | | | 0.741 | | | | [symbol] | | [symbol] | [symbol] |
| (Tukey's post-hoc) | | | | | | | | | | | |
| vs. Group 3 | | | | | | | | | | | |
| ANOVA | | | | [symbol] | | | | [symbol] | | [symbol] | [symbol] |
| (Tukey's post-hoc) | | | | | | | | | | | |
| vs. Group 4 | | | | | | | | | | | |
| ANOVA | | | | [symbol] | | | | [symbol] | | [symbol] | [symbol] |
| (Tukey's post-hoc) | | | | | | | | | | | |
| vs. Group 5 | | | | | | | | | | | |
| Group 5 | | | | | | | | | | | |
| AAV -5 | | | | | | | | | | | |
| Guite 1 + 2 | | | | | | | | | | | |
| (5 × 10\hat{ }9 of | | | | | | | | | | | |
| each guide/knee) | | | | | | | | | | | |
| IA, Ix (D 0); | | | | | | | | | | | |
| 1 | 3.68 | 0.00 | [symbol] | [symbol] | [symbol] | 0.00 | [symbol] | 0.00 | 3.70 | 0.32 | [symbol] |
| 2 | [symbol] | 0.00 | 3.68 | [symbol] | [symbol] | 0.00 | 3.67 | −0.01 | 3.70 | 0.32 | [symbol] |
| 3 | [symbol] | 0.00 | 3.71 | [symbol] | [symbol] | 0.00 | 3.70 | −0.01 | 3.72 | 0.31 | [symbol] |
| 4 | [symbol] | 0.00 | [symbol] | 0.03 | [symbol] | 0.00 | 3.65 | −0.01 | [symbol] | [symbol] | [symbol] |
| 5 | [symbol] | 0.00 | [symbol] | 0.04 | [symbol] | 0.00 | [symbol] | 0.00 | 3.68 | [symbol] | [symbol] |
| 6 | [symbol] | 0.00 | [symbol] | 0.02 | [symbol] | 0.00 | 3.69 | 0.01 | [symbol] | [symbol] | [symbol] |
| 7 | [symbol] | 0.00 | [symbol] | [symbol] | [symbol] | 0.00 | 3.74 | 0.00 | [symbol] | [symbol] | [symbol] |
| 8 | 3.72 | 0.00 | 3.69 | [symbol] | [symbol] | 0.00 | [symbol] | 0.00 | [symbol] | [symbol] | [symbol] |
| 9 | [symbol] | 0.00 | 3.73 | 0.03 | 3.72 | 0.00 | [symbol] | 0.01 | [symbol] | [symbol] | [symbol] |
| 10 | [symbol] | 0.00 | 3.74 | 0.00 | [symbol] | 0.00 | [symbol] | −0.01 | 3.75 | [symbol] | [symbol] |
| 11 | [symbol] | 0.00 | [symbol] | [symbol] | [symbol] | 0.00 | [symbol] | 0.00 | [symbol] | [symbol] | [symbol] |
| 12 | [symbol] | 0.00 | [symbol] | [symbol] | [symbol] | 0.00 | [symbol] | 0.00 | 3.67 | [symbol] | 0.01 |
| 13 | [symbol] | 0.00 | 3.71 | [symbol] | [symbol] | 0.00 | [symbol] | 0.00 | [symbol] | [symbol] | 0.04 |
| 14 | 3.68 | 0.00 | [symbol] | 0.02 | 3.68 | 0.00 | [symbol] | 0.01 | 3.70 | [symbol] | 0.03 |
| Extra | 3.72 | [symbol] | 3.72 | [symbol] | [symbol] | [symbol] | 2.72 | [symbol] | 3.70 | [symbol] | [symbol] |
| Mean | 3.69 | [symbol] | [symbol] | [symbol] | [symbol] | [symbol] | [symbol] | [symbol] | 3.72 | [symbol] | 0.02 |
| SE | [symbol] | [symbol] | 0.01 | [symbol] | [symbol] | [symbol] | 0.01 | [symbol] | [symbol] | [symbol] | [symbol] |
| Group 3 | | | | | | | | | | | |
| PBS | | | | | | | | | | | |
| IA, Ix (D 0) | | | | | | | | | | | |
| 1 | [symbol] | [symbol] | 3.89 | 0.04 | 3.85 | [symbol] | 3.85 | 0.00 | [symbol] | 0.05 | 0.05 |
| 2 | [symbol] | 0.00 | 3.78 | 0.00 | 3.78 | [symbol] | 3.78 | −0.02 | [symbol] | 0.01 | [symbol] |
| 3 | [symbol] | 0.00 | 3.72 | 0.04 | 3.68 | [symbol] | 3.68 | [symbol] | 3.75 | [symbol] | [symbol] |
| 4 | 3.72 | 0.00 | [symbol] | 0.05 | 3.72 | [symbol] | 3.74 | [symbol] | 3.79 | [symbol] | 0.12 |
| 5 | 3.62 | 0.00 | 2.82 | [symbol] | 3.82 | [symbol] | 3.82 | [symbol] | [symbol] | [symbol] | 0.01 |
| 6 | [symbol] | 0.00 | 3.68 | [symbol] | 3.68 | [symbol] | 3.66 | [symbol] | [symbol] | 0.06 | [symbol] |
| 7 | 3.78 | 0.00 | 3.78 | [symbol] | 3.78 | [symbol] | 3.78 | [symbol] | [symbol] | [symbol] | 0.01 |
| 8 | [symbol] | 0.00 | 3.76 | 0.04 | [symbol] | [symbol] | 3.73 | [symbol] | [symbol] | [symbol] | 0.08 |
| 9 | [symbol] | 0.00 | [symbol] | 0.04 | [symbol] | [symbol] | 3.65 | [symbol] | [symbol] | [symbol] | [symbol] |
| 10 | [symbol] | 0.00 | [symbol] | [symbol] | 3.58 | [symbol] | 3.38 | [symbol] | 3.59 | 0.01 | [symbol] |
| 11 | 3.63 | 0.00 | [symbol] | [symbol] | [symbol] | [symbol] | [symbol] | [symbol] | [symbol] | 0.03 | [symbol] |
| 12 | [symbol] | 0.00 | 3.79 | [symbol] | 3.79 | [symbol] | 3.77 | −0.02 | 3.82 | 0.03 | [symbol] |
| 13 | 3.56 | 0.00 | 3.58 | 0.02 | 3.56 | [symbol] | 3.56 | [symbol] | 3.61 | [symbol] | [symbol] |
| 14 | 2.73 | 0.00 | 3.78 | [symbol] | [symbol] | [symbol] | 3.72 | [symbol] | [symbol] | 0.02 | [symbol] |
| Mean | [symbol] | [symbol] | 3.72 | 0.02 | 3.78 | [symbol] | 3.78 | [symbol] | [symbol] | [symbol] | [symbol] |
| SE | [symbol] | [symbol] | 0.02 | [symbol] | 0.02 | [symbol] | [symbol] | [symbol] | [symbol] | [symbol] | [symbol] |
| ANOVA | | | | [symbol] | | | | [symbol] | | 0.214 | [symbol] |
| (Tukey's post-hoc) | | | | | | | | | | | |
| vs. Group 4 | | | | | | | | | | | |
| ANOVA | | | | [symbol] | | | | 0.975 | | 0.168 | [symbol] |
| (Tukey's post-hoc) | | | | | | | | | | | |
| vs. Group 5 | | | | | | | | | | | |
| Group 4 | | | | | | | | | | | |
| AAV-4 | | | | | | | | | | | |
| Scramble Vector | | | | | | | | | | | |
| (1 × 10\hat{ }10 | | | | | | | | | | | |
| particles/knee) | | | | | | | | | | | |
| IA, Ix (D 0) | | | | | | | | | | | |
| 1 | 3.67 | 0.00 | [symbol] | 0.03 | 3.67 | [symbol] | [symbol] | [symbol] | 3.70 | [symbol] | 0.07 |
| 2 | [symbol] | 0.00 | [symbol] | 0.03 | [symbol] | [symbol] | [symbol] | −0.02 | [symbol] | 0.04 | −0.03 |
| 3 | 3.72 | 0.00 | 3.74 | 0.02 | 3.72 | [symbol] | [symbol] | 0.01 | 3.75 | [symbol] | 0.05 |
| 4 | 3.61 | 0.00 | [symbol] | 0.02 | [symbol] | [symbol] | 3.61 | [symbol] | [symbol] | 0.03 | 0.03 |
| 5 | 3.68 | 0.00 | 3.71 | 0.03 | [symbol] | [symbol] | [symbol] | [symbol] | 3.71 | 0.03 | 0.03 |
| 6 | 3.84 | 0.00 | [symbol] | [symbol] | [symbol] | [symbol] | 3.82 | −0.02 | [symbol] | [symbol] | [symbol] |
| 7 | 3.72 | 0.00 | 3.74 | 0.02 | [symbol] | [symbol] | 3.72 | [symbol] | [symbol] | [symbol] | 0.03 |
| 8 | 3.70 | 0.00 | [symbol] | 0.12 | [symbol] | [symbol] | [symbol] | 0.01 | 3.72 | 0.02 | [symbol] |
| 9 | [symbol] | 0.00 | [symbol] | 0.01 | [symbol] | [symbol] | [symbol] | 0.01 | 3.83 | 0.03 | 0.05 |
| 10 | [symbol] | 0.00 | 3.72 | [symbol] | [symbol] | [symbol] | 3.72 | [symbol] | 3.76 | 0.04 | [symbol] |
| 11 | [symbol] | 0.00 | [symbol] | [symbol] | [symbol] | [symbol] | [symbol] | [symbol] | [symbol] | 0.02 | [symbol] |
| 12 | 3.63 | 0.00 | 3.64 | [symbol] | 3.63 | [symbol] | 3.63 | [symbol] | 3.69 | 0.06 | 0.02 |
| 13 | 3.59 | 0.00 | [symbol] | [symbol] | [symbol] | [symbol] | 3.57 | −0.02 | 3.62 | 0.03 | [symbol] |
| 14 | 3.68 | 0.00 | [symbol] | [symbol] | 3.68 | [symbol] | [symbol] | [symbol] | 3.71 | 0.03 | 0.02 |
| Extra | 3.74 | 0.00 | [symbol] | [symbol] | [symbol] | [symbol] | [symbol] | −0.02 | [symbol] | [symbol] | [symbol] |
| Mean | 3.70 | 0.00 | 3.72 | [symbol] | 3.70 | [symbol] | 3.69 | [symbol] | 3.73 | 0.04 | 0.02 |
| SE | 0.02 | 0.00 | 0.82 | [symbol] | [symbol] | [symbol] | 0.02 | [symbol] | [symbol] | [symbol] | 0.01 |
| ANOVA | | | | [symbol] | | | | [symbol] | | 0.962 | [symbol] |
| (Tukey's post-hoc) | | | | | | | | | | | |
| vs. Group 5 | | | | | | | | | | | |

[symbol]

indicates data missing or illegible when filed

Moribund or Found Dead Animals

If animals were found dead no samples were taken. For animals needing to be euthanized, regardless of reason, samples were taken as they would at necropsy (see Necropsy Information section). Animals on health assessment may be given SC fluids as well as hydrogel and food on the bottom of the cage.

Study Group Designations

| | | | | Dose Level | | | | | |
|---|---|---|---|---|---|---|---|---|---|
| | | | | (particles/ | Dose | | Dose Conc. | Dose | |
| Grp | N | MSU | Treatment | knee) | Vol. | | (particles/ml) | Route | Regimen |
| 1 | 14 | Yes | AAV-6 | 1 × 10¹⁰ | 10 | μl | 1 × 10¹² | IA (right | 1x (Day 0) |
| | | | Scramble | | | | | knee) | |
| | | | Vector | | | | | | |
| 2 | 14 | Yes | AAV-6 | 5 × 10⁹ | 10 | μl | 1 × 10¹² | IA (right | 1x (Day 0) |
| | | | Guide 1 + 2 | (of each | | | | knee) | |
| | | | | guide) | | | | | |
| 3 | 14 | Yes | PBS | — | 10 | μl | — | IA (right | 1x (Day 0) |
| | | | | | | | | knee) | |
| 4 | 14 | Yes | AAV-5 | 1 × 10¹⁰ | 10 | μl | 1 × 10¹² | IA (right | 1x (Day 0) |
| | | | Scramble | | | | | knee) | |
| | | | Vector | | | | | | |
| 5 | 14 | Yes | AAV-5 | 5 × 10⁹ | 10 | μl | 1 × 10¹² | IA (right | 1x (Day 0) |
| | | | Guide 1 + 2 | (of each | | | | knee) | |
| | | | | guide) | | | | | |

Study Calendar

| Mon | Tue | Wed | Thu | Fri | Sat | Sun |
|---|---|---|---|---|---|---|
| May 4, 2020 | May 5, 2020 | May 6, 2020 | May 7, 2020 | May 8, 2020 | May 9, 2020 | May 10, 2020 |
| Day −10 | Day −9 | Day −8 | Day −7 | Day −6 | Day −5 | Day −4 |
| Distribute | | | | Acclimate to VF | | |
| animals on arrival | | | | | | |
| into groups for | | | | | | |
| acclimation | | | | | | |
| May 11, 2020 | May 12, 2020 | May 13, 2020 | May 14, 2020 | May 15, 2020 | May 16, 2020 | May 17, 2020 |
| Day −3 | Day −2 | Day −1 | Day 0 | Day 1 | Day 2 | Day 3 |
| Acclimate to VF | Acclimate to VF | Weigh & | Timed Dose, | Timed Caliper, | Weigh | |
| | | Randomize, | Timed Caliper, | Timed Behavior | | |
| | | Baseline Caliper, | Timed Behavior | | | |
| | | Baseline | | | | |
| | | Behavior. Shave | | | | |
| | | Knees | | | | |
| May 18, 2020 | May 19, 2020 | May 20, 2020 | May 21, 2020 | May 22, 2020 | May 23, 2020 | May 24, 2020 |
| Day 4 | Day 5 | Day 6 | Day 7 | Day 8 | Day 9 | Day 10 |
| | | Weigh, Shave | Timed MSU, | | | |
| | | Knees? | Timed Caliper, | | | |
| | | | Timed Behavior, | | | |
| | | | Timed Necropsy | | | |

Materials

| Name | Supplier | Cat #* |
|---|---|---|
| MSU crystals | Invivogen | Tlrl-msu-25 |
| Vehicle (PBS) | Corning | 21-031-CV |
| Gene | SgRNA | AA06-CCPCTRO1-AD01-200 |
| Gene | SgRNA | AA06-MCP001682-AD01-2-200A |
| Gene | SgRNA | AA06-MCP001682-AD01-2-200B |
| Gene | SgRNA | AA05 -CCPCTROI-AD01-200 |
| Gene | SgRNA | AA05-MCP001682-AD01-2-200-9 |
| Gene | SgRNA | AA05-MCP001682-AD01-2-200-6 |
| Isoflurane | VetOne | 502017 |
| Syringes & Needles | BD | As needed |
| Serum Separator Tubes | Greiner Bio-One | #450472 (via Fisher) |
| (if needed) | | |
| Li Hep Mini-Collect | Greiner Bio-One | #450480 (via Fisher) |
| (if needed) | | |
| EDTA Mini-Collect | Greiner Bio-One | #450477 (via Fisher) |
| (if needed) | | |
| K3EDTA (if needed) | Covidien | #8881311149 (via Fisher) |

Test Article and Vehicle Information

| | | | | | Doses | Compound | | TOTAL |
|---|---|---|---|---|---|---|---|---|
| | | | ml/ | Days of | Per | Needed | 25% | Needed |
| Grp | Cpmd | N | animal | Dosing | Day | (ml) | Overage | (ml) |
| 1 | AAV-6 Scramble Vector | 14 | 0.01 | 1 | 1 | 0.14 | 0.04 | 0.18 |
| 2 | AAV-6 Guide 1 + 2 | 14 | 0.01 | 1 | 1 | 0.14 | 0.04 | 0.18 |
| 3 | PBS | 14 | 0.01 | 1 | 1 | 0.14 | 0.04 | 0.18 |
| 4 | AAV-5 Scramble Vector | 14 | 0.01 | 1 | 1 | 0.14 | 0.04 | 0.18 |
| 5 | AAV-5 Guide 1 + 2 | 14 | 0.01 | 1 | 1 | 0.14 | 0.04 | 0.18 |

Unformulated Test Article Storage Conditions:

AAV-6 Scrambled Vector (Group 1): −80 C.
AAV-6 Guide 1 + 2 (Group 2): −80 C.
PBS (Group 3): 4 C.
AAV-5 Scrambled Vector (Group 4): −80 C.
AAV-5 Guide 1 + 2 (Group 5): −80 C.

Vehicle Information:

AAV-6 Scrambled Vector (Group 1): Sterile PBS (w/o Ca & Mg)
AAV-6 Guide 1 + 2 (Group 2): Sterile PBS (w/o Ca & Mg)
PBS (Group 3): Sterile PBS (w/o Ca & Mg)
AAV-5 Scrambled Vector (Group 4): Sterile PBS (w/o Ca & Mg)
AAV-5 Guide 1 + 2 (Group 5): Sterile PBS (w/o Ca & Mg)

Test Article Formulation Instructions & Calculations:

AAV-6 Scrambled Vector (Group 1): Dilute stock to appropriate
concentration using sterile PBS (See AAV Preparation section)
AAV-6 Guide 1 + 2 (Group 2): Dilute stock to appropriate
concentration using sterile PBS (See AAV Preparation section)
PBS (Group 3): Use sterile PBS (See AAV Preparation section)
AAV-5 Scrambled Vector (Group 4): Dilute stock to appropriate
concentration using sterile PBS (See AAV Preparation section)
AAV-5 Guide 1 + 2 (Group 5): Dilute stock to appropriate
concentration using sterile PBS (See AAV Preparation section)

Dosing Formulations and Vehicle Storage & Stability:

AAV-6 Scrambled Vector (Group 1): Dilute just prior to injecting
AAV-6 Guide 1 + 2 (Group 2): Dilute just prior to injecting
AAV-5 Scrambled Vector (Group 4): Dilute just prior to injecting
AAV-5 Guide 1 + 2 (Group 5): Dilute just prior to injecting

Disposition of Test Articles Following Dosing:

AAV-6 Scrambled Vector (Group 1): Discard formulations, retain stock
solution at −80 C. for future studies
AAV-6 Guide 1 + 2 (Group 2): Discard formulations, retain stock solution
at −80 C. for future studies
AAV-5 Scrambled Vector (Group 4): Discard formulations, retain stock
solution at −80 C. for future studies
AAV-5 Guide 1 + 2 (Group 5): Discard formulations, retain stock solution
at −80 C. for future studies

Live Phase

Live Phase Data Collection

| Type | Study Day | Grp (An) | Details |
|---|---|---|---|
| Body Weight | Day −1, 2, 6 | All | |
| Knee Caliper | Day −1 (Baseline) | All | Right Knee |
| | Day 0 (6 hr) | | Change from |
| | Day 1 (24 hour) | | baseline |
| | Day 7 (0 hr, 6 hour) | | |
| Von Frey | Day −1 (Baseline) | All | Right foot only |
| | Day 0 (6 hr) | | |
| | Day 1 (24 hour) | | |
| | Day 7 (0 hr, 6 hour) | | |

Necropsy Information

The animals were necropsied after the final behavioral testing on study day 7 (approx. 6 h post-MSU). At necropsy, the animals were bled by cardiac puncture to exsanguination and euthanized by cervical dislocation for tissue collection. Whole blood was processed for serum (≥200 μL/mouse), which was stored frozen at −80° C. for shipment to the study sponsor. Right (injected) and left (normal) knees from all animals were collected (skin, muscle, and feet were removed while keeping the knee joint intact). The joints were flash-frozen straight in 15-mL conical tubes for shipment to the sponsor.

| | |
|---|---|
| Sacrifice Schedule: | Study Day 7 (6 hours post MSU): |
| | ALL ANIMALS |
| Method of Euthanasia: | Bleed by cardiac puncture to exsanguinate |
| | followed by cervical dislocation. |
| Time Points: | After final behavior time point (6 hours |
| | post MSU) |

Necropsy Sample Collection:

| | Process | Anti- | | Final | Storage | Disposi- |
|---|---|---|---|---|---|---|
| Type | For | Coag | Gr/An | Volume | Condition | tion |
| Cardiac | Serum | N/A | All | ≥200 μl | −80 C., | Ship to |
| Puncture | | | | | Epps | Sponsor |
| Whole Blood | | | | | | |

Necropsy Tissue Sample Collection:

| Type | Gr/An | Details | Storage Condition | Disposition |
|---|---|---|---|---|
| Right (Injected) | All | Remove skin & | Flash freeze | Ship to |
| Knee | | muscle keeping | straight, place | Sponsor |
| | | knee joint intact, | in 15 ml conical | |
| | | remove foot | | |
| Left (Normal) | All | Remove skin & | Flash freeze | Ship to |
| Knee | | muscle keeping | straight, place | Sponsor |
| | | knee joint intact, | in 15 ml conical | |
| | | remove foot | | |

Statistical Analysis

Data were entered into Microsoft Excel and means and standard errors (SE) for each group were determined. The groups were compared using a one-way analysis of variance (ANOVA) or a repeated measures (RM) ANOVA with a Tukey's post-hoc analysis. ANOVA were performed using Prism v8.0.2 software (GraphPad). Unless indicated, BBP performs statistical analysis on raw (untransformed) data only. Statistical tests make certain assumptions regarding the data's normality and homogeneity of variance, and further analysis may be required if testing resulted in violations of these assumptions. Significance for all tests was set at p<0.050 with p values rounded to the third decimal place.

AAV Preparation

Standard Operating Procedure for Preparing Virus for Injection

Create a working stock that contains 5×10\hat{ }11 vg per ml for each construct (Guide 1, Guide 2). Note that for equivalence, the scramble control group needs to be injected with a total of 1×10\hat{ }10 copies of the scrambled vector. Diluent (PBS) will be used in the vehicle control group.

Materials

AA06-CCPCTR01-AD01-200: Scramble control AAV-6 particles,
100 microliter solution containing 5 × 10\hat{ }12 vg per ml.
AA06-MCP001682-AD01-2-200-Guide 1: AAVPrimeTMparticles,
AAV-6 containing Guide 1, 100 microliter solution containing
5 × 10\hat{ }12 vg per ml.
AA06-MCP001682-AD01-2-200-Guide 2: AAVPrimeTMparticles,
AAV-6 containing Guide 2, 100 microliter solution containing
5 × 10\hat{ }12 vg per ml.
AA05-CCPCTR01-AD01-200: Scramble control AAV-5 particles,
100 microliter solution containing >5 × 10\hat{ }12 vg per ml.
AA05-MCP001682-AD01-2-200-Guide 1: AAVPrimeTMparticles,
AAV-5 containing Guide 1, 100 microliter solution
containing >5 × 10\hat{ }12 vg per ml.
AA05-MCP001682-AD01-2-200-Guide 2: AAVPrimeTMparticles,
AAV-5 containing Guide 2, 100 microliter solution
containing >5 × 10\hat{ }12 vg per ml.

Procedure

Group 1: AAV-6 Scramble Control—Aliquot 400 microliters of sterile-filtered Ca- and Mg-free PBS into a sterile Eppendorf tube. Add 100 microliters (equivalent to 5×10\hat{ }11 vg) of the stock AA06-CCPCTR01-AD01-200. This results in a 0.5 ml volume of working stock containing 1×10\hat{ }12 vg/ml of the AAV-5 scramble control. Injection of 10 microliters of this solution into the knee joint delivers 1×10\hat{ }10 vg of the AAV-6 scramble control.

Group 2: Active AAV-6 Guide 1+2—Aliquot 800 microliters of sterile-filtered Ca- and Mg-free PBS into a sterile Eppendorf tube. Add 100 microliters (equivalent to 5×10\hat{ }11 vg) of each of the two active AAV-6 constructs—this means 100 microliters (equivalent to 5×10\hat{ }11 vg) of AA06-MCP001682-AD01-2-200-a and 100 microliters (equivalent to 5×10\hat{ }11 vg) of the stock AA06-MCP001682-AD01-2-200-b. This results in a 1 ml volume of working stock containing 5×10\hat{ }11 vg/ml for each of the two guides. Injection of 10 microliters of this solution into the knee joint delivers 5×10\hat{ }9 vg for each of the two AAV-6 guides.

Group 3: PBS—The sterile-filtered Ca- and Mg-free PBS used to dilute the virus stock served as the vehicle control for this study. It was dosed at 10 microliters per knee joint.

Group 4: AAV-5 Scramble Control—Aliquot 400 microliters of sterile-filtered Ca- and Mg-free PBS into a sterile Eppendorf tube. Add 100 microliters (equivalent to 5×10\hat{ }11 vg) of the stock AA05-CCPCTR01-AD01-200. This results in a 0.5 ml volume of working stock containing 1×10\hat{ }12 vg/ml of the AAV-5 scramble control. Injection of 10 microliters of this solution into the knee joint delivered 1×10\hat{ }10 vg of the AAV-5 scramble control.

Group 5: Active AAV-6 Guide 1+2—Aliquot 800 microliters of sterile-filtered Ca- and Mg-free PBS into a sterile Eppendorf tube. Add 100 microliters (equivalent to 5×10\hat{ }11 vg) of each of the two active AAV-6 constructs—this means 100 microliters (equivalent to 5×10\hat{ }11 vg) of AA05-MCP001682-AD01-2-200-a and 100 microliters (equivalent to 5×10\hat{ }11 vg) of the stock AA05-MCP001682-AD01-2-200-b. This results in a 1 ml volume of working stock containing 5×10\hat{ }11 vg/ml for each of the two guides. Injection of 10 microliters of this solution into the knee joint delivered 5×10\hat{ }9 vg for each of the two AAV-6 guides.

qPCR

Snap-frozen synovial tissues resected en bloc (including distal femur and proximal tibia) and placed in RLT buffer. Homogenised using Cyrolys Evolution tissue homogenizer (“HARD” programme cycle). RNA extracted using RNeasy or RNeasy Plus kits followed by QIAshredder (from QIAGEN). RNA quantified using Nanonstring. cDNA reverse transcribed and qPCR performed using mouse-specific primers for IL-1 beta, beta-actin and RPL13.

Results

As shown in FIG. 6, PBS control mice had mean body weight gain of 4.3% (1.1 g) over the course of the study. Body weight gain did not differ statistically across groups (Table 8, Table 10).

As shown in FIG. 7A, knee caliper measurements in all groups peaked 6 hours post-dose on study day 0, returned to baseline by 24 hours post-dose, and then peaked again 6 hours post-MSU on study day 7. Knee caliper change from baseline did not differ statistically across groups over time (Table 12). As shown in FIG. 7B, knee caliper change AUC for days −1 through 7 did not differ statistically across groups (Table 8, Table 12).

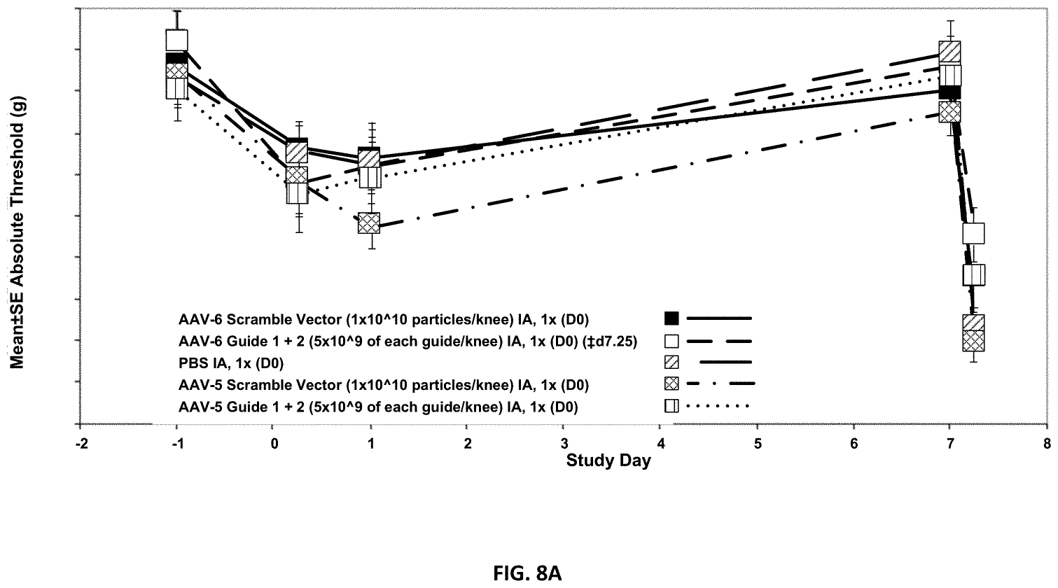

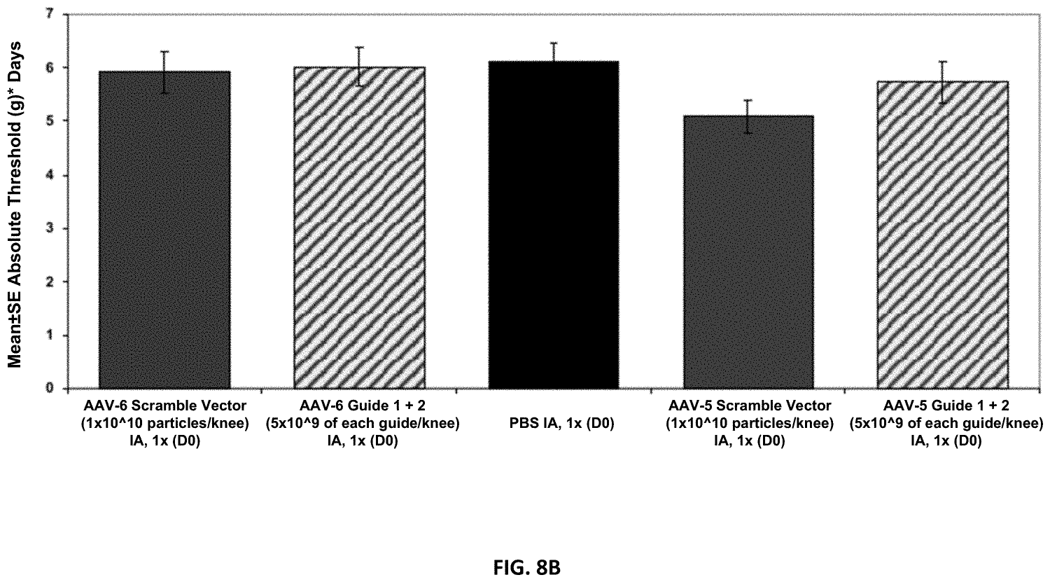

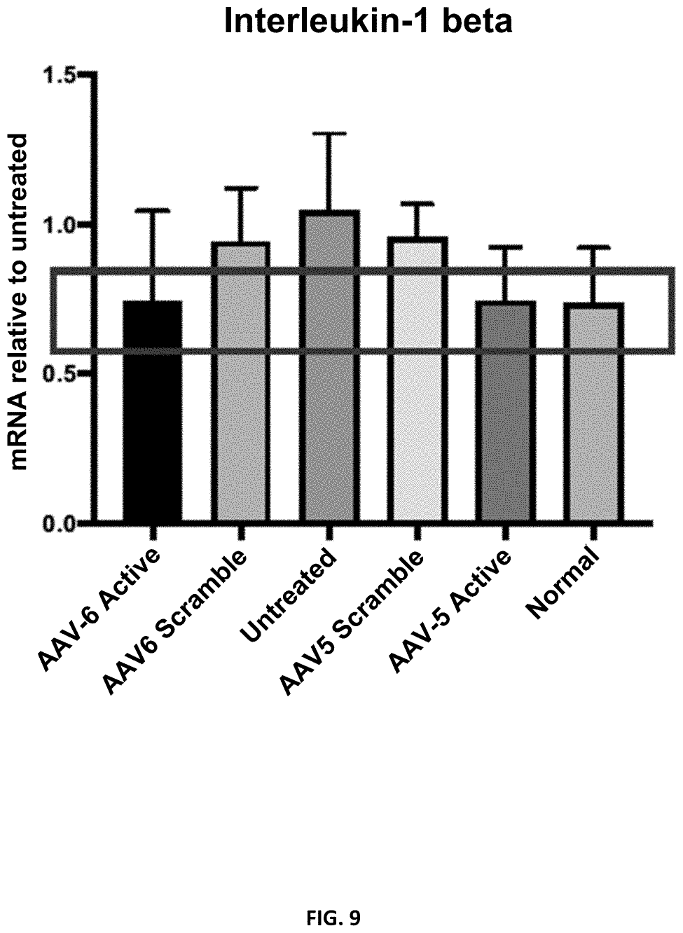

As shown in FIG. 8A, von Frey absolute thresholds decreased slightly in all groups following IA dosing on study day 0 and then trended toward baseline on study day 7 before decreasing sharply following IA injection of MSU. There were no statistical differences over time between the AAV scramble vector control groups and the PBS control group. Mice treated with AAV-6 (Guide 1+2) had von Frey absolute thresholds that were increased 6 hours post-MSU on day 7 as compared to the control groups; the increase in von Frey absolute thresholds at this time point nearly reached statistical significance as compared to the AAV-6 scramble vector and PBS control groups (p=0.051 and p=0.075, respectively) and was statistically significant as compared to the AAV-5 scramble vector control group (p=0.025). Mice treated with AAV-5 (Guide 1+2) had von Frey absolute thresholds that did not differ statistically from the control groups over time (Table 11). As shown in FIG. 8B, von Frey absolute threshold AUC for days −1 through 7 did not differ statistically across groups (Table 8, Table 11).

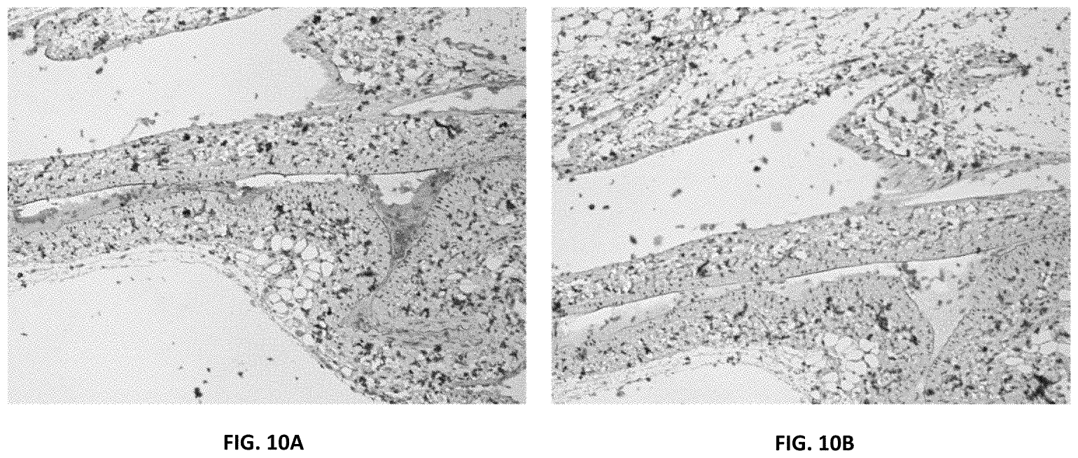

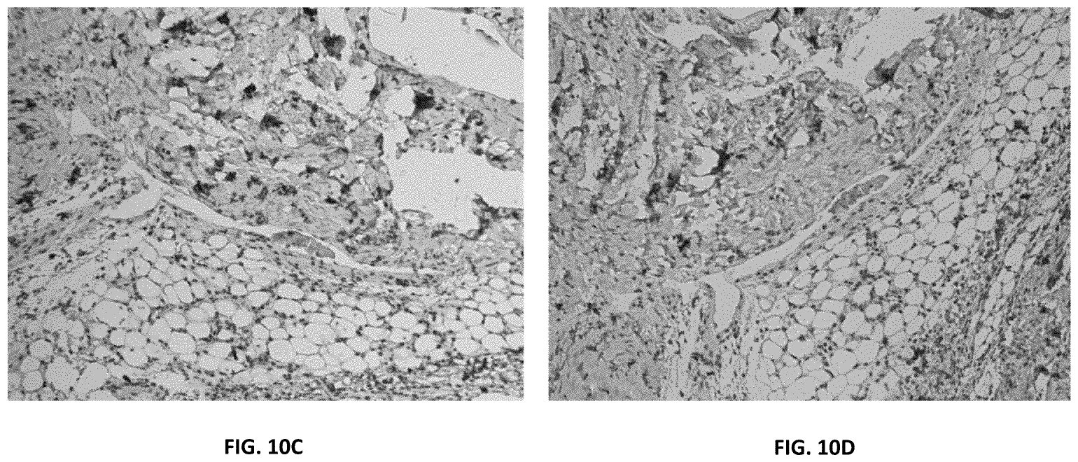

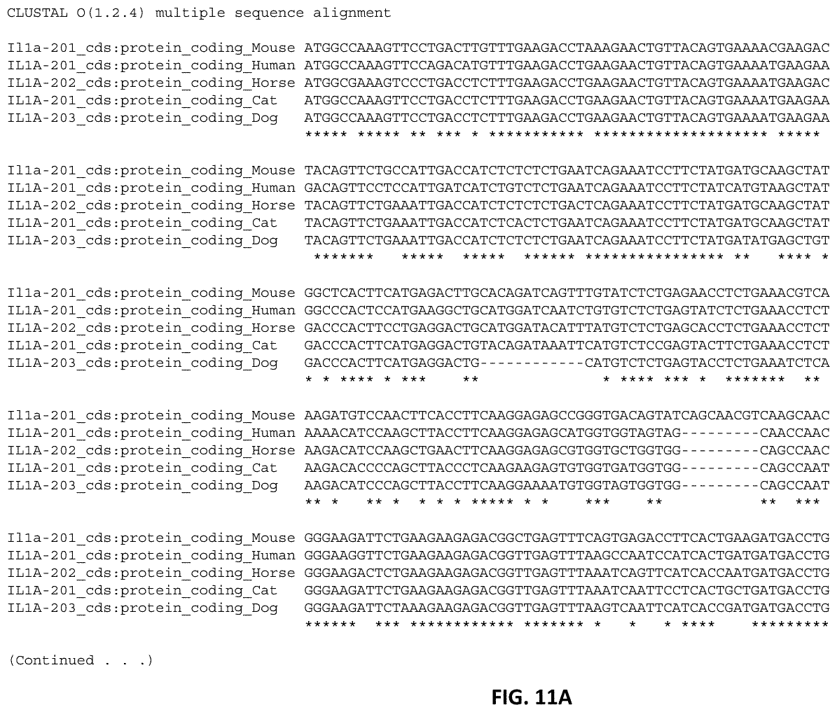

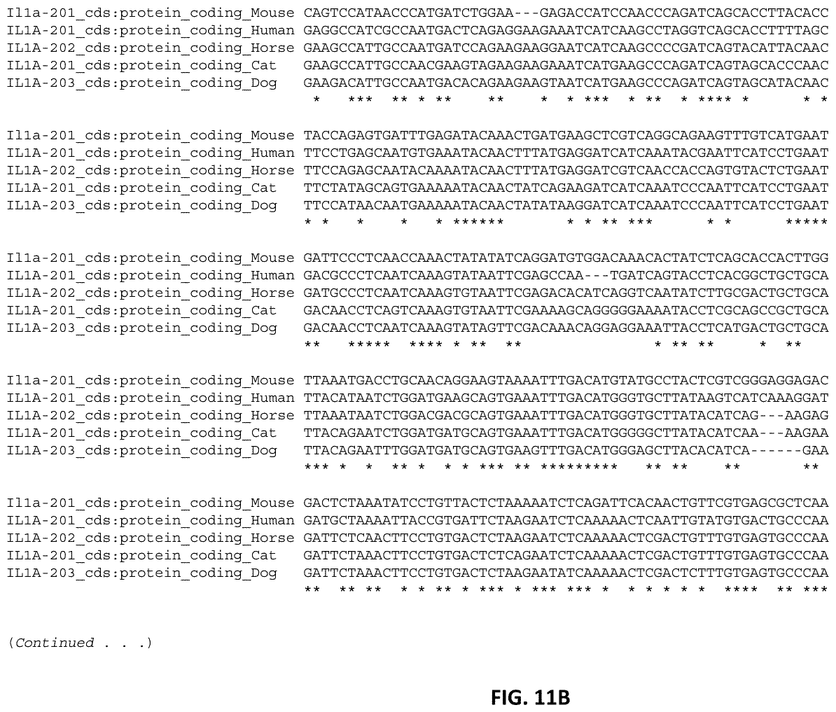

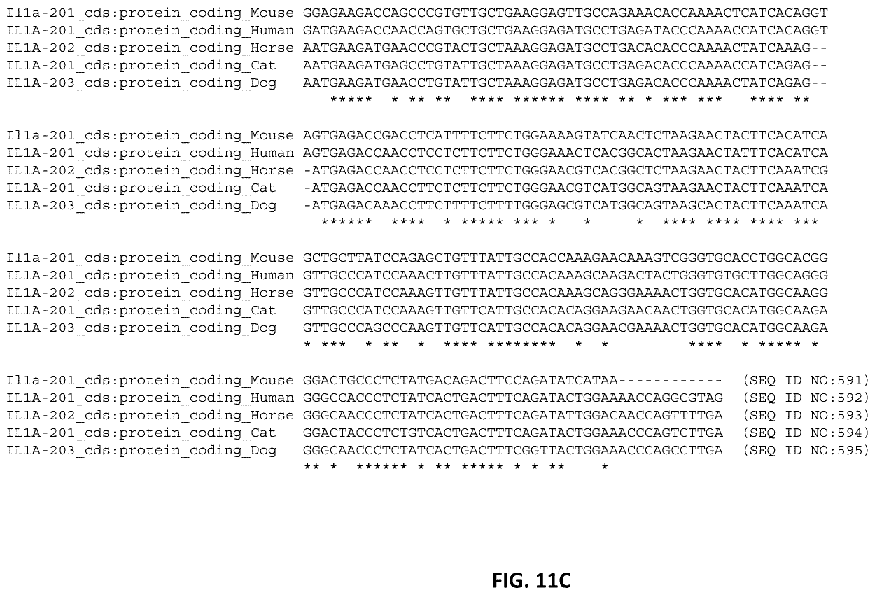

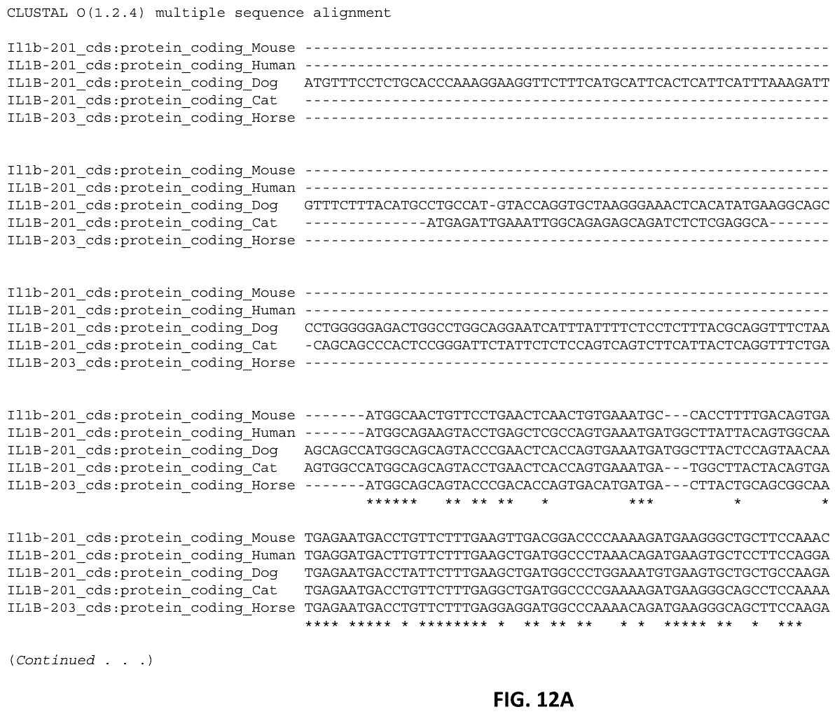

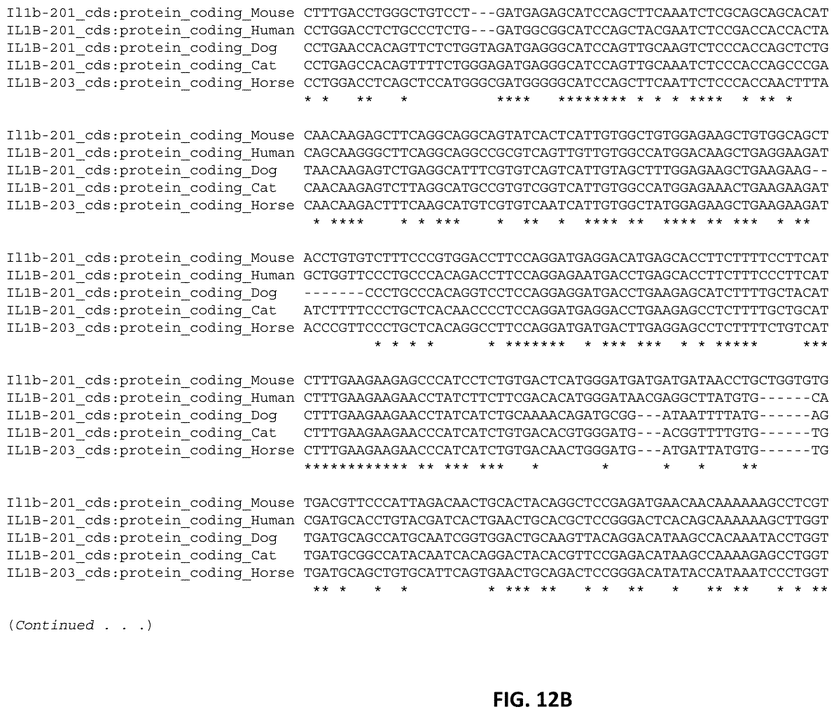

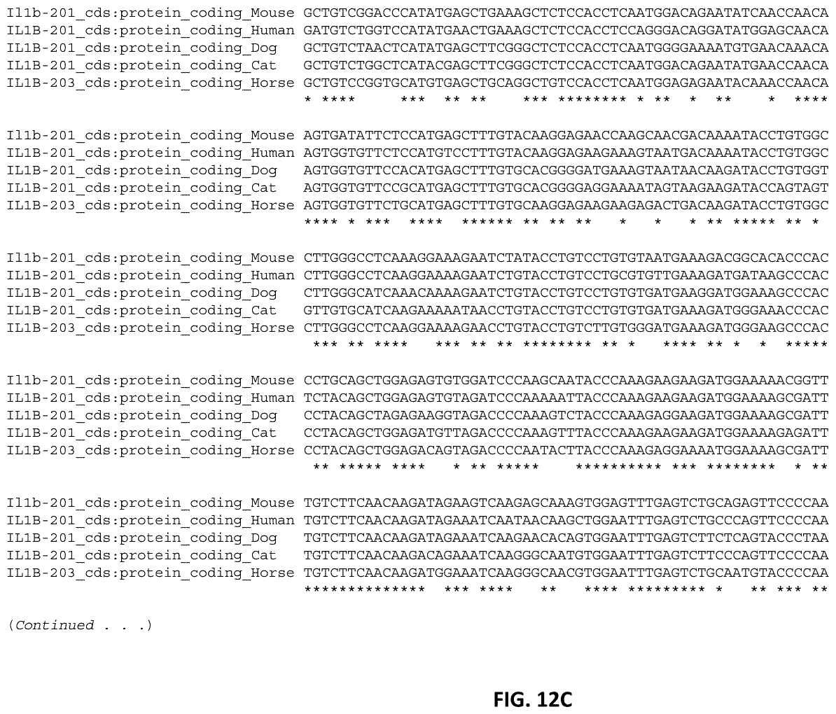

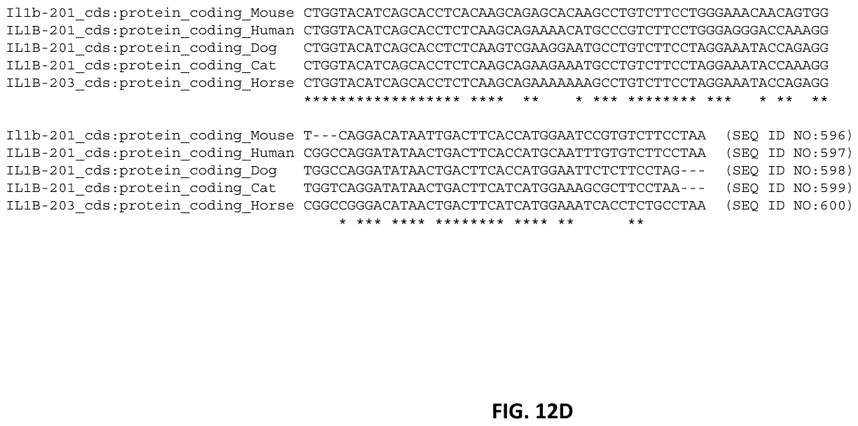

As shown in FIG. 10, immunohistochemistry data for murine IL-1β in synovial tissue showed reduced IL-1β expression in CRISPR-treated animals. (A) In MSU injected animals pre-treated with PBS there is robust expression of IL-1β (brown staining). This effect is not seen in CRISPR-treated animals (panel C). The absence of IL-1β (brown staining) in CRISPR-treated animals resembles negative antibody controls (panels B & D). All images are 10× original magnification.

Discussion & Conclusions

Mice treated IA (1× on day 0) with AAV-6 (Guide 1+2: 5×10⁹ vg/guide per knee) showed a statistically significant reduction in referred pain, as measured by von Frey testing, 6 hours after MSU injection on day 7 as compared to mice injected IA with AAV-5 scramble vector (p=0.025) with results being nearly significant as compared to the AAV-6 scramble vector and PBS control groups (p=0.051 and p=0.075, respectively). AUC calculations for von Frey assessments did not differ statistically across groups. Animal body weight gain and knee swelling did not differ statistically across groups. All animals survived to study termination.

### Example 6. Guide RNA Design

Guide RNAs targeting human IL-1α and IL-1β (Table 15) were designed according to the following procedure:

1. Identify appropriate genome assembly and gene model (Tools: Ensemble, UCSC Genome Browser);

2. Identify key functional domains to map out targeting window (Tools: Ensemble; Literature);

3. Generate list of all possible guide RNAs across key exons (Tools: Ensemble, UCSC, InDelphi);

4. Rank guides based on ML-predicted frameshifting score and exclude poor performers;

5. Exclude guides <5 bp from intron:exon boundaries and with homopolynucleotide tracts of 6×T's or greater;

6. Determine on-target (Doench 2016) and off-target (Hsu 2013) metrics for each guide (Tools: UCSC, Deskgen);

7. Filter out guides with poor on- and off-target scores to generate final list; and

8. Rank based on frameshift index.

Guide RNAs targeting cat, dog, or horse IL-1α and IL-1β (Table 16) were designed according to the following procedure:
1. Identify appropriate genome assembly and gene model (Tools: Ensemble, UCSC Genome Browser)
2. Identify key functional domains to map out targeting window (Tools: Ensemble; Literature)
3. Retrieve coding sequence from appropriate exons and relevant flanking intronic sequences (Tools: Ensemble. APE)
4. Generate list of all possible guide RNAs across key exons (Tools: Ensemble, InDelphi)
5. Rank guides based on ML-predicted frameshifting score and exclude poor performers
6. Exclude guides <5 bp from intron:exon boundaries and with homopolynucleotide tracts of 6×T's or greater
7. Determine off-target metrics for each guide (Tools: Cas Off-Finder, Excel)
8. Filter out guides with poor off-target scores to generate final list
9. Rank based on frameshift index.

Gene transcript information for all species considered are included in Table 17.

TABLE 15

| | SEQ | | | | Frame- | | | | |
|---|---|---|---|---|---|---|---|---|---|
| Gene | ID | | Chromo- | | shift | Doench | Hsu | | |
| symbol | NO: | Species | some | Genome | (%) | 2016 | 2013 | gRNA | PAM |
| IL1B | 627 | Human | chr2 | hg38 | 80.1 | 55 | 76 | AGCTGGATGCCGCCATCCAG | AGG |
| IL1B | 628 | Human | chr2 | hg38 | 78.5 | 44 | 71 | ACCACTACAGCAAGGGCTTC | AGG |
| IL1B | 629 | Human | chr2 | hg38 | 75.4 | 63 | 92 | CATGGCCACAACAACTGACG | CGG |
| IL1B | 630 | Human | chr2 | hg38 | 74 | 60 | 97 | GGTGGTCGGAGATTCGTAGC | TGG |
| IL1B | 631 | Human | chr2 | hg38 | 87.6 | 55 | 61 | CTACAGCAAGGGCTTCAGGC | AGG |
| IL1B | 632 | Human | chr2 | hg38 | 85.6 | 61 | 58 | GACCTCTGCCCTCTGGATGG | CGG |
| IL1B | 633 | Human | chr2 | hg38 | 85.2 | 35 | 62 | CTCTCCGCAGTGCTCCTTCC | AGG |
| IL1B | 634 | Human | chr2 | hg38 | 78.5 | 50 | 62 | CATTCTCCTGGAAGGTCTGT | GGG |
| IL1A | 635 | Human | chr2 | hg38 | 92.1 | 70 | 87 | ATGGTGGTAGTAGCAACCAA | CGG |
| IL1A | 636 | Human | chr2 | hg38 | 88.3 | 51 | 88 | GCCATAGCTTACATGATAGA | AGG |
| IL1A | 637 | Human | chr2 | hg38 | 87 | 68 | 88 | CAGAGACAGATGATCAATGG | AGG |
| IL1A | 638 | Human | chr2 | hg38 | 87 | 50 | 74 | ATTCAGAGACAGATGATCAA | TGG |
| IL1A | 639 | Human | chr2 | hg38 | 83.1 | 53 | 91 | TGGTGGTAGTAGCAACCAAC | GGG |
| IL1A | 640 | Human | chr2 | hg38 | 85.7 | 60 | 67 | CTCCAGGTCATCATCAGTGA | TGG |

TABLE 16

| | SEQ | | | | | Frame- | CasOff- | | |
|---|---|---|---|---|---|---|---|---|---|
| Gene | ID | | Chromo- | | | shift | Finder | | |
| symbol | NO: | Species | some | Exon | Genome | (%) | total | gRNA | PAM |
| IL1A | 641 | Horse | chr15 | 4 | EquCab3.0 | 80.9 | 21 | TGGTGCTGGTGGCAGCCAAC | GGG |
| IL1A | 642 | Horse | chr15 | 4 | EquCab3.0 | 79.2 | 14 | CTTCTTCAGAGTCTTCCCGT | TGG |
| IL1A | 643 | Horse | chr15 | 3 | EquCab3.0 | 74.9 | 37 | TTACCTGAGTCAGAGAGAGA | TGG |
| IL1A | 644 | Horse | chr15 | 4 | EquCab3.0 | 74.6 | 11 | AAGCTATGACCCACTTCCTG | AGG |
| IL1A | 645 | Horse | chr15 | 4 | EquCab3.0 | 71.5 | 14 | CCATGCAGTCCTCAGGAAGT | GGG |
| IL1A | 646 | Dog | chr17 | 3 | canFam4 | 75.7 | 7 | ACATGCAGTCCTCATGAAGT | GGG |
| IL1A | 647 | Dog | chr17 | 3 | canFam4 | 73.8 | 10 | GACATCCCAGCTTACCTTCA | AGG |
| IL1A | 648 | Dog | chr17 | 3 | canFam4 | 72.6 | 10 | GAGCTGTGACCCACTTCATG | AGG |
| IL1A | 649 | Dog | chr17 | 3 | canFam4 | 79.2 | 14 | CATTTTCCTTGAAGGTAAGC | TGG |
| IL1A | 650 | Dog | chr17 | 3 | canFam4 | 87 | 16 | TGGTAGTGGTGGCAGCCAAT | GGG |
| IL1A | 651 | Dog | chr17 | 3 | canFam4 | 76.8 | 18 | ATTTTCCTTGAAGGTAAGCT | GGG |
| IL1A | 652 | Dog | chr17 | 3 | canFam4 | 79.1 | 23 | CTTCTTTAGAATCTTCCCAT | TGG |
| IL1A | 653 | Dog | chr17 | 3 | canFam4 | 71.4 | 23 | GGATGTCTTTGAGATTTCAG | AGG |
| IL1A | 654 | Dog | chr17 | 3 | canFam4 | 88.5 | 28 | GTGGTAGTGGTGGCAGCCAA | TGG |
| IL1A | 655 | Dog | chr17 | 3 | canFam4 | 78.7 | 31 | CACTACCACATTTTCCTTGA | AGG |
| IL1A | 656 | Cat | chrA3 | 3 | Fc9.0 | 73.3 | 12 | GGTAAGCTGGGGTGTCTTAG | AGG |
| IL1A | 657 | Cat | chrA3 | 3 | Fc9.0 | 74.3 | 13 | ATTCCTCACTGCTGATGACC | TGG |
| IL1A | 658 | Cat | chrA3 | 3 | Fc9.0 | 86.7 | 15 | TGGTGATGGTGGCAGCCAAT | GGG |
| IL1A | 659 | Cat | chrA3 | 3 | Fc9.0 | 79.3 | 20 | CTTCCAGGTCATCAGCAGTG | AGG |
| IL1B | 660 | Horse | chr15 | 4 | EquCab3.0 | 87.6 | 27 | TGAAAGTCTTGTTGTAAAGT | TGG |
| IL1B | 661 | Horse | chr15 | 4 | EquCab3.0 | 79.6 | 15 | GACCTCAGCTCCATGGGCGA | TGG |
| IL1B | 662 | Horse | chr15 | 4 | EquCab3.0 | 78.5 | 13 | CTGGATGCCCCCATCGCCCA | TGG |
| IL1B | 663 | Horse | chr15 | 4 | EquCab3.0 | 75.4 | 17 | CCCCATCGCCCATGGAGCTG | AGG |
| IL1B | 664 | Horse | chr15 | 4 | EquCab3.0 | 75.2 | 12 | AAGTCTTGTTGTAAAGTTGG | TGG |
| IL1B | 665 | Dog | chr17 | 3 | canFam4 | 82.1 | 2 | CGTGTCAGTCATTGTAGCTT | TGG |
| IL1B | 666 | Dog | chr17 | 3 | canFam4 | 74.2 | 11 | CCACAGTTCTCTGGTAGATG | AGG |
| IL1B | 667 | Dog | chr17 | 3 | canFam4 | 80 | 13 | AGACCTGAACCACAGTTCTC | TGG |
| IL1B | 668 | Dog | chr17 | 3 | canFam4 | 80 | 14 | CCTCATCTACCAGAGAACTG | TGG |
| IL1B | 669 | Dog | chr17 | 3 | canFam4 | 80 | 15 | TCAGACTCTTGTTACAGAGC | TGG |
| IL1B | 670 | Cat | chrA3 | 2 | Fc9.0 | 82.4 | 8 | GTAGTAAGCCATCATTTCAC | TGG |
| IL1B | 671 | Cat | chrA3 | 4 | Fc9.0 | 82 | 4 | GAGTCTTAGGCATGCCGTGT | CGG |
| IL1B | 672 | Cat | chrA3 | 4 | Fc9.0 | 81.8 | 14 | AAACCTGAGCCACAGTTTTC | TGG |
| IL1B | 673 | Cat | chrA3 | 2 | Fc9.0 | 80.7 | 17 | ATCATTTCACTGGTGAGTTC | AGG |
| IL1B | 674 | Cat | chrA3 | 4 | Fc9.0 | 80.4 | 18 | ACTCTTGTTGTCGGGCTGGT | GGG |
| IL1B | 675 | Cat | chrA3 | 4 | Fc9.0 | 80.1 | 14 | GACTCTTGTTGTCGGGCTGG | TGG |
| IL1B | 676 | Cat | chrA3 | 4 | Fc9.0 | 76.7 | 24 | CCTCATCTCCCAGAAAACTG | TGG |
| IL1B | 677 | Cat | chrA3 | 3 | Fc9.0 | 76 | 23 | TGAGAATGACCTGTTCTTTG | AGG |
| IL1B | 678 | Cat | chrA3 | 4 | Fc9.0 | 75.7 | 5 | TAAGACTCTTGTTGTCGGGC | TGG |
| IL1B | 679 | Cat | chrA3 | 4 | Fc9.0 | 74.3 | 18 | AACCTGAGCCACAGTTTTCT | GGG |

TABLE 17

| Gene | Species | Ensemble | Transcript | Transcript_ID | Location |
|---|---|---|---|---|---|
| IL1A | Human | ENSG00000115008 | IL1A-201 | ENST00000263339.4 | Chromosome 2: 112,773,925-112,784,493 |
| IL1A | Mouse | ENSMUSG00000027399 | Il1a-201 | ENSMUST00000028882.1 | Chromosome 2: 129,299,610-129,309,972 |
| IL1A | Cat | ENSFCAG00000008095 | IL1A-201 | ENSFCAT00000008097.5 | Chromosome A3: 105,113,193-105,121,209 |
| IL1A | Dog | ENSCAFG00000007245 | IL1A-201 | ENSCAFT00000045928.3 | Chromosome 17: 36,970,573-36,978,736 |
| IL1A | Horse | ENSECAG00000023727 | IL1A-202 | ENSECAT00000025409.2 | Primary_assembly 15: 16,360,700-16,369,812 |
| IL1B | Human | ENSG00000125538 | IL1B-201 | ENST00000263341.7 | Chromosome 2: 112,829,751-112,836,779 |
| IL1B | Mouse | ENSMUSG00000027398 | Il1b-201 | ENSMUST00000028881.13 | Chromosome 2: 129,364,570-129,371,139 |
| IL1B | Cat | ENSFCAG00000005899 | IL1B-201 | ENSFCAT00000005904.5 | Chromosome A3: 105,049,334-105,056,407 |
| IL1B | Dog | ENSCAFG00000007249 | IL1B-201 | ENSCAFT00000011613.4 | Chromosome 17: 37,019,329-37,024,176 |
| IL1B | Horse | ENSECAG00000000051 | IL1B-202 | ENSECAT00000061518.2 | Primary_assembly 15: 16,418,738-16,444,697 |

### Example 7. Immunohistochemistry for Murine IL-1β

Immunohistochemistry was performed on Murine synovial tissue to detect IL-1β according to the following protocol:

Reagents & Preparation

1. 10×PBS with 0.5% (v/v) Tween-20

2. Sterile PBS

3. IHC buffer

4. Primary antibody (goat anti-mouse IL-1; AF-401-NA, R&D Systems, Inc.) reconstituted to a final concentration of 0.2 mg/ml in sterile PBS.
a. Short-term storage at +4C
b. Long-term storage at −20 to −70C

5. Reconstitute the control antibody (normal goat IgG; AB-108-C, R&D Systems Inc.) at a final concentration of 1 mg/ml in sterile PBS.
a. Short-term storage at +4C
b. Long-term storage at −20 to −70C

6. Secondary antibody (HRP conjugated donkey anti-goat IgG; ab6885; Abcam) comes as reconstituted product
a. Short-term storage at +4C
b. Long-term storage at −20 to −70C

7. Peroxidase block (BLOXALL reagent, Vector Laboratories)

8. Normal horse serum, diluted to 2.5% (v/v) (Impress Polymer Kit; Vector Laboratories)

9. DAB chromogen

Method

Antigen retrieval was performed for 1 hour (manual or automated, user preference), and then samples were transferred to PBS for short-term storage. Peroxidase block was performed for 10 minutes at room temperature, and samples were subsequently washed in IHC buffer for 5 mins. Samples were blocked with control horse serum for 60 minutes at room temperature, and exposed to primary antibody (1:100 or 1:200 diluted in 1×PBS-Tween) for 2 hours at room temperature. Samples were washed in IHC buffer twice for 5 mins (each wash). Samples were then exposed to secondary antibody (1:500 diluted in 1×PBS-Tween) for 1 hour, and washed in IHC buffer twice for 5 mins (each wash). Detection was performed with DAB chromogen for 30 seconds. Counterstaining was performed with Mayer's haematoxylin (6 minutes), and samples were returned through graded alcohol series to xylene. DPX mountant was applied, and a coverslip was attached.

As shown in FIG. 10, immunohistochemistry data for murine IL-1β in synovial tissue showed reduced IL-1β expression in CRISPR-treated animals. (A) In MSU injected animals pre-treated with PBS there is robust expression of IL-1β (brown staining). This effect is not seen in CRISPR-treated animals (panel C). The absence of IL-1β (brown staining) in CRISPR-treated animals resembles negative antibody controls (panels B & D). All images are 10× original magnification. (A) and (B) are adjacent sections taken from the same joint in the same animal, with (A) showing tissue labeled specifically for IL-1 beta, and (B) showing tissue labeled with the negative (isotope) control antibody. Differences in staining reflect demonstrable IL-1 beta expression in MSU injected animals pre-treated with PBS in this animal (e.g., a positive control animal pre-treated with PBS, then challenged with MSU crystal). (C) and (D) are similarly adjacent sections, but from an animal that was pre-treated with the CRISPR editing virus prior to MSU injection. (C) There is no obvious staining for IL-1 beta in the section treated with IL-1 antibody, and (D) the same negative pattern is seen in the section treated with the negative (isotope) control antibody. Without wishing to be bound by any particular theory, this confirms that there is no detectable IL-1 beta expression in the synovium of CRISPR-treated animals.

### Example 8. Design and Validation of CRISPR/Cas9 RNA Guides Against Canine and Human Interleukin-1 Alpha (IL-1α) and Interleukin-1 Beta (IL-1β)

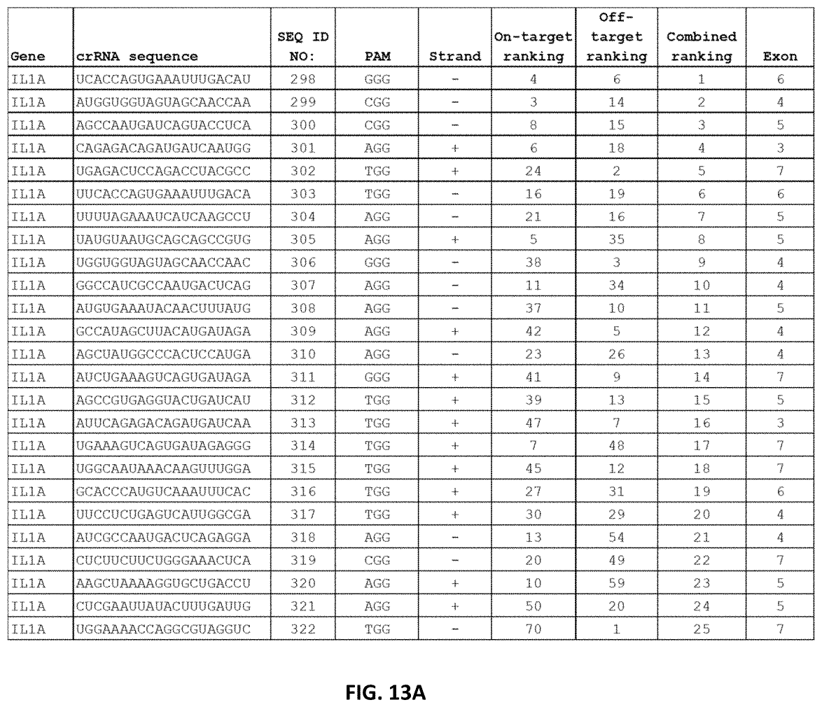

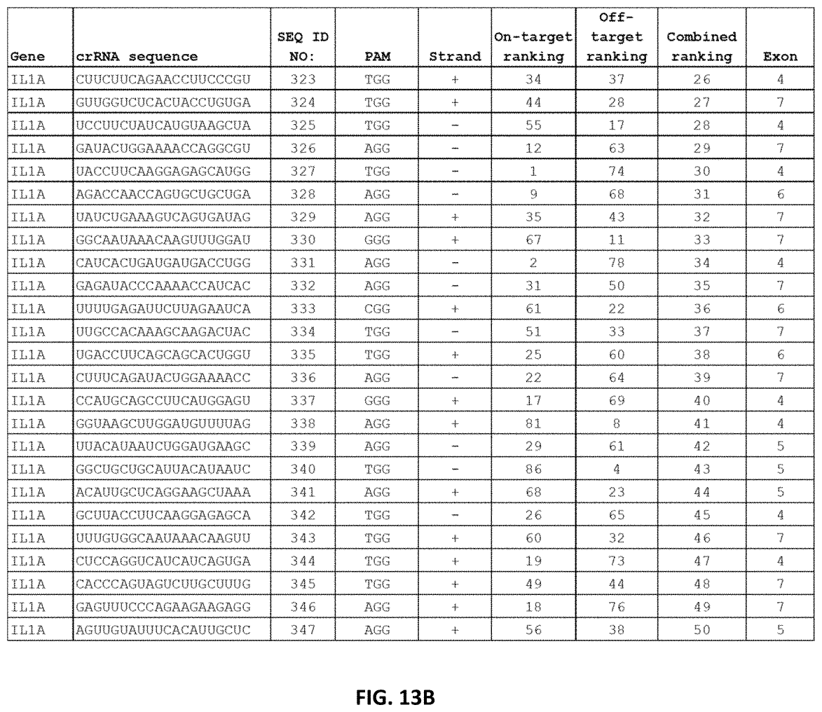

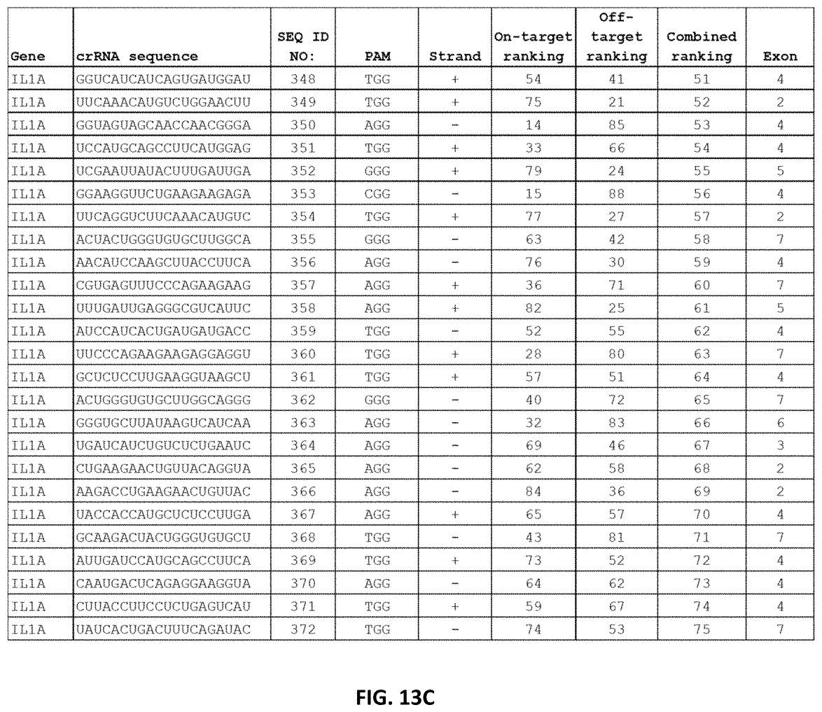

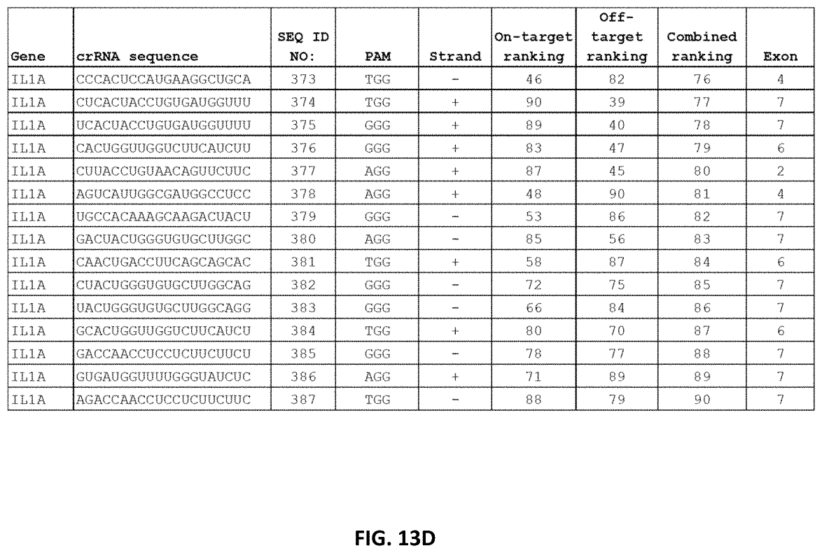

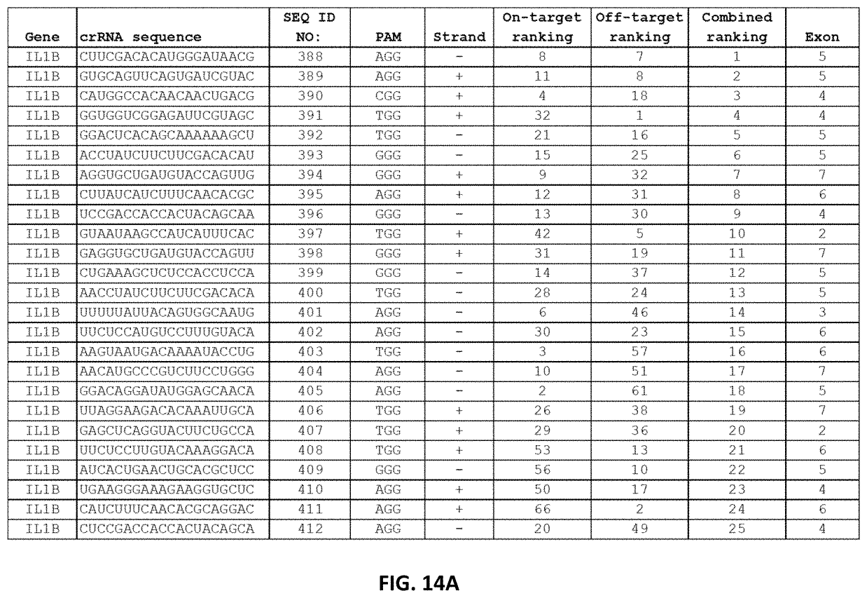

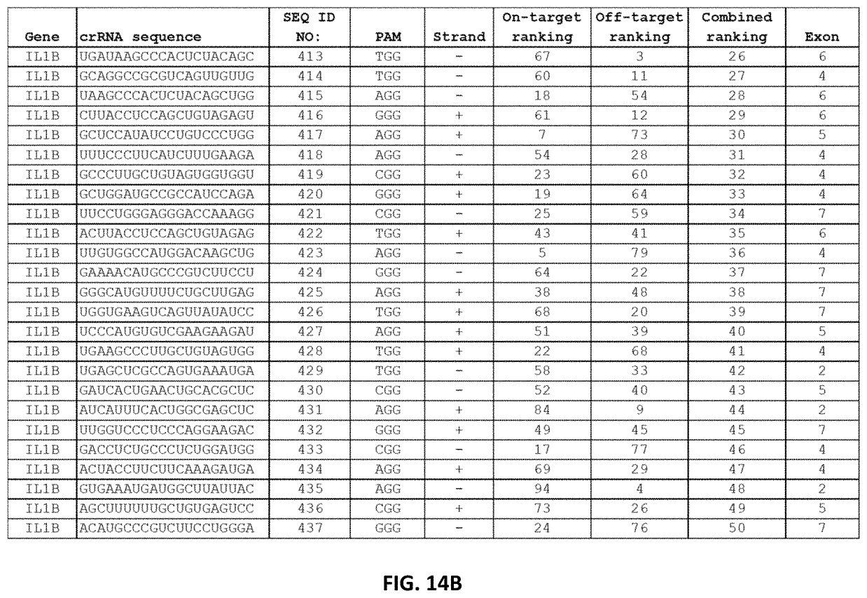

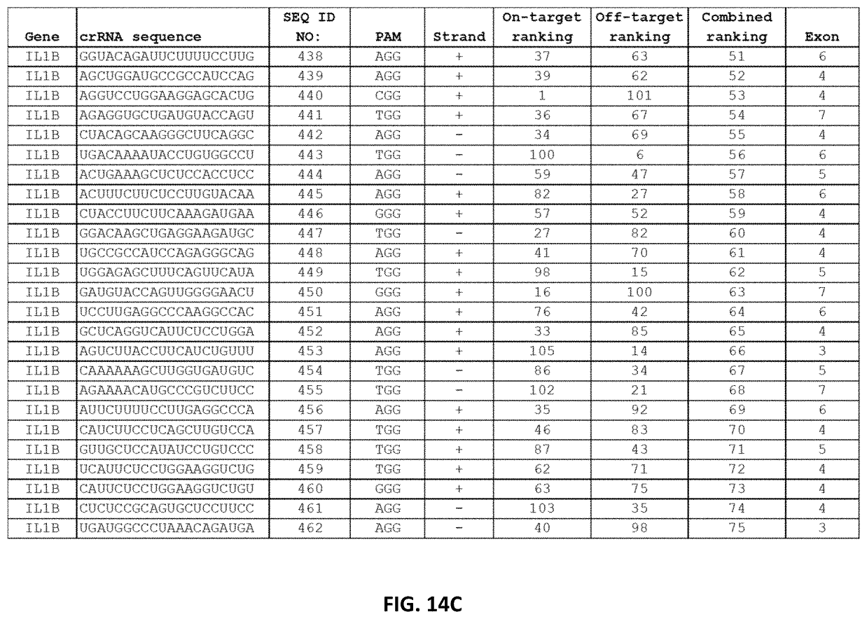

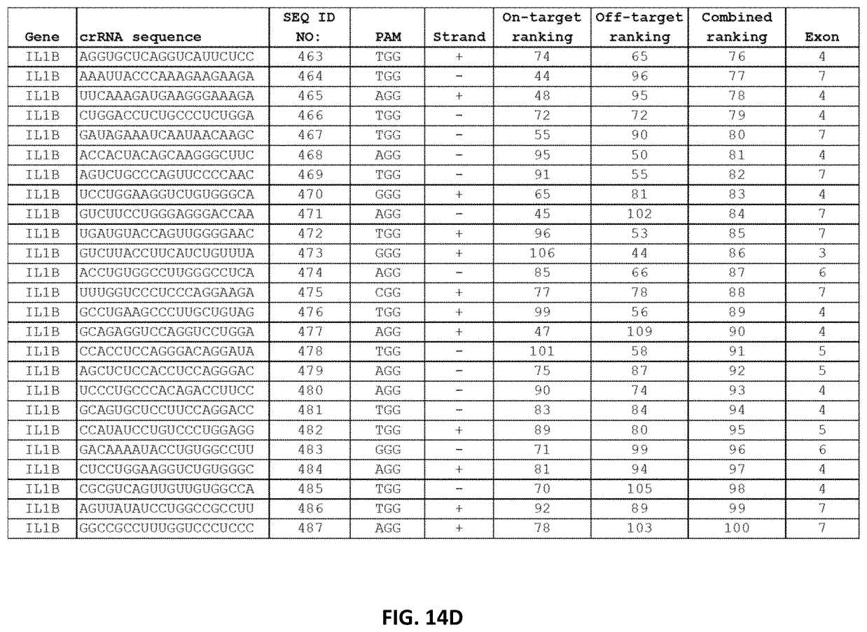

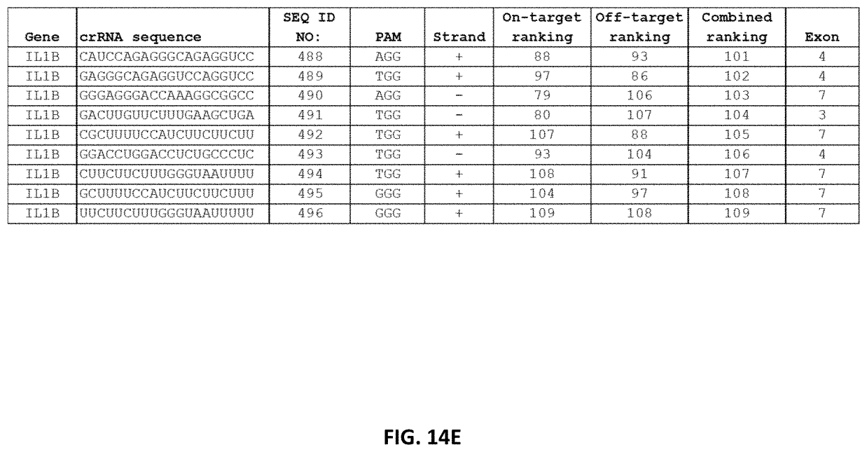

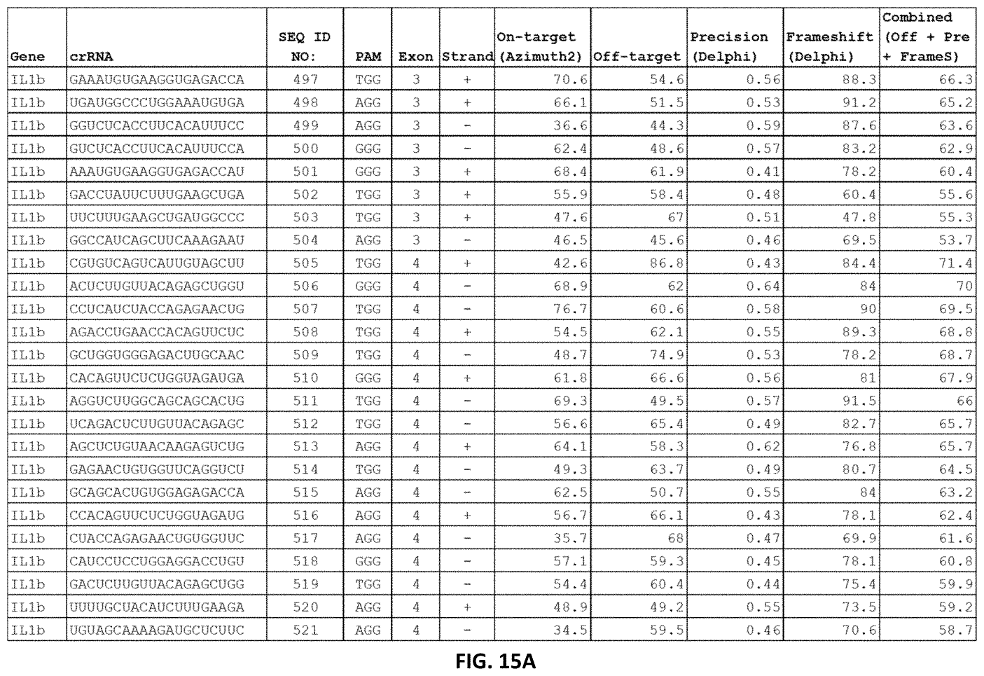

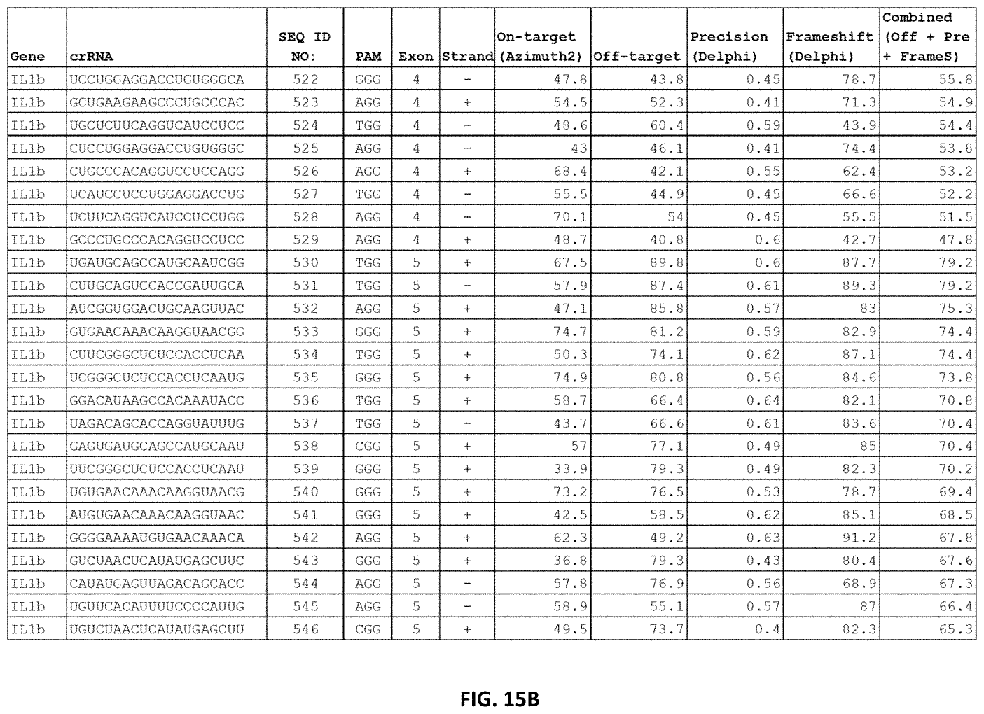

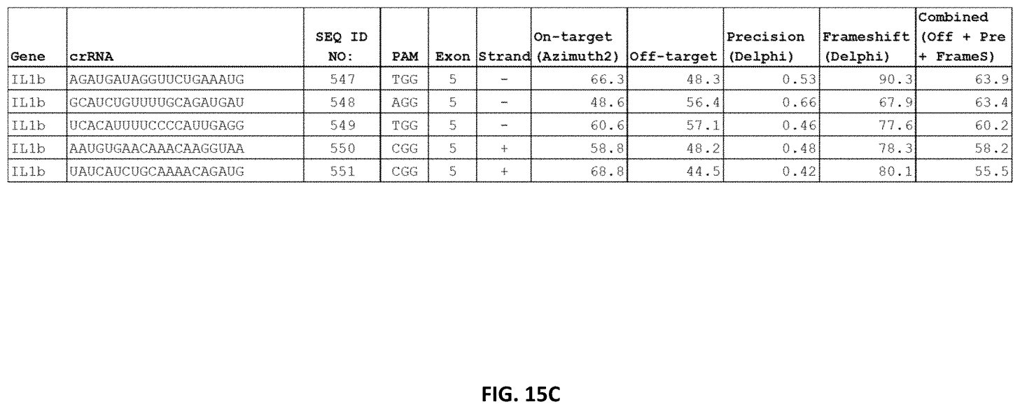

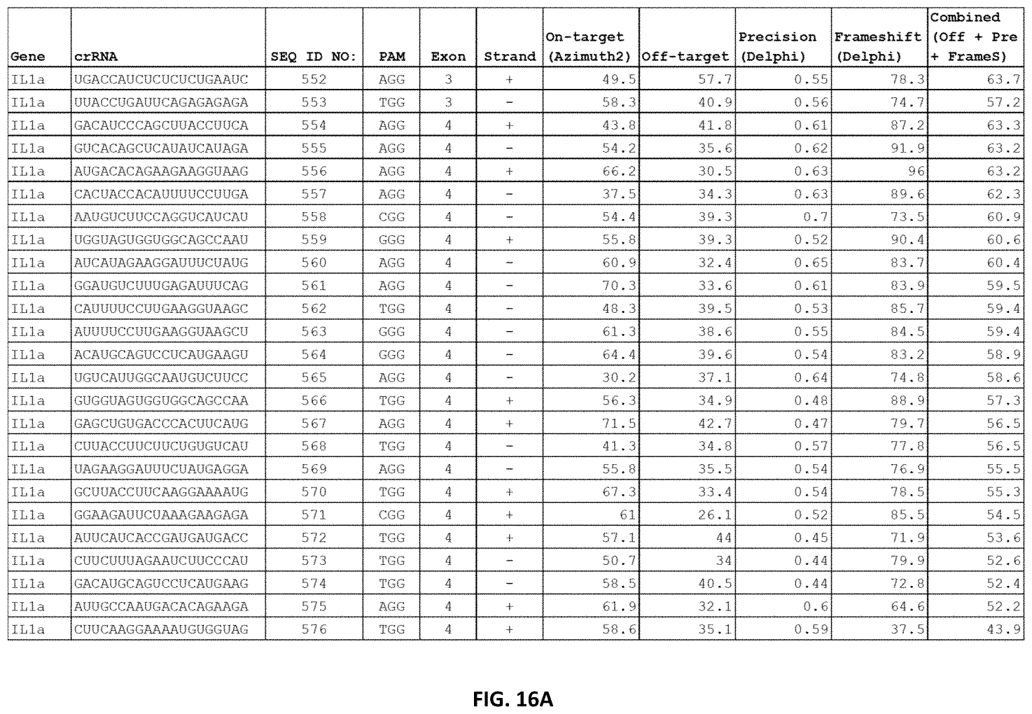

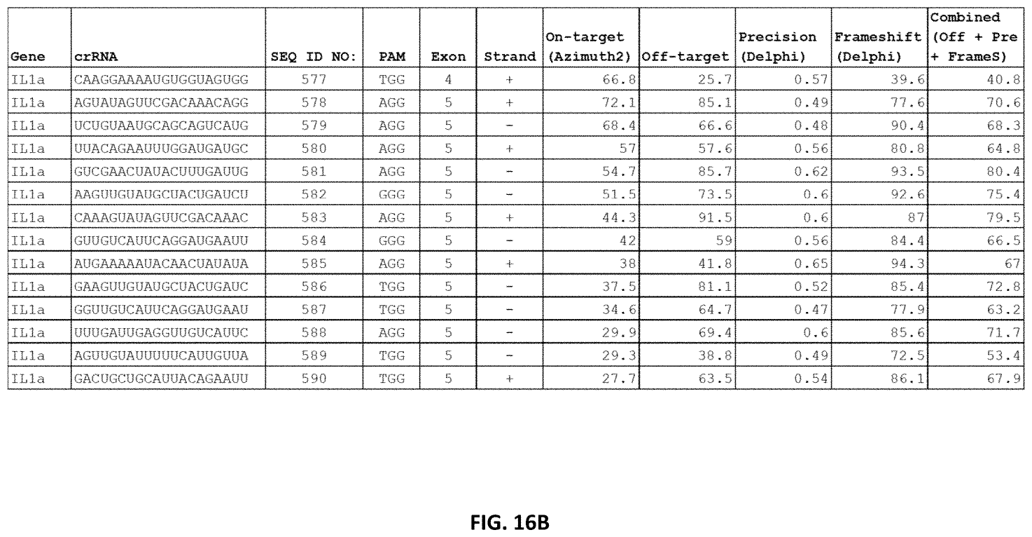

Potential crRNA sequences were identified for various exons of the human and canine interleukin-1 alpha (IL-1α) and interleukin-1 beta (IL-1β) genes. FIGS. 13A-13D show a ranked list of crRNA sequences identified from exons 2-7 of the human IL-1α gene. FIGS. 14A-14E show a ranked list of crRNA sequences identified from exons 2-7 of the human IL-1β gene. FIGS. 15A-15C show crRNA sequences identified from exons 3-5 of the canine IL-1β gene. FIGS. 16A-16B show a crRNA sequences identified from exons 3-5 of the canine IL-1α gene.

Publicly accessible genomes (human, hg38; dog, CanFam3.1), collapsed gene models (merged Ensembl/Havana), tissue-specific exon expression (gtexportal.org) and various gRNA models were then used to select two to five individual crRNA sequences per gene, targeting canine and human interleukin-1 alpha (IL-1α) and interleukin-1 beta (IL-1β). The following gRNA design rules were applied:

1. The gRNA target region was limited to the first 5-50% of the coding sequence (CDS).
2. Single gRNAs were ranked according to maximal on-target editing using Azimuth 2.0 model (10.1038/nbt.3437) and minimal off-target editing using Cutting Frequencing Determination (CFD) (10.1038/nbt.3437) and the specificity score from Hsu et al. (10.1038/nbt.2647).
3. Highly ranked sgRNA with high frameshift frequencies (>75%) and uniform DNA repair outcomes (>0.48) as predicted by inDelphi (10.1038/s41586-018-0686-x) were selected for in vitro synthesis.

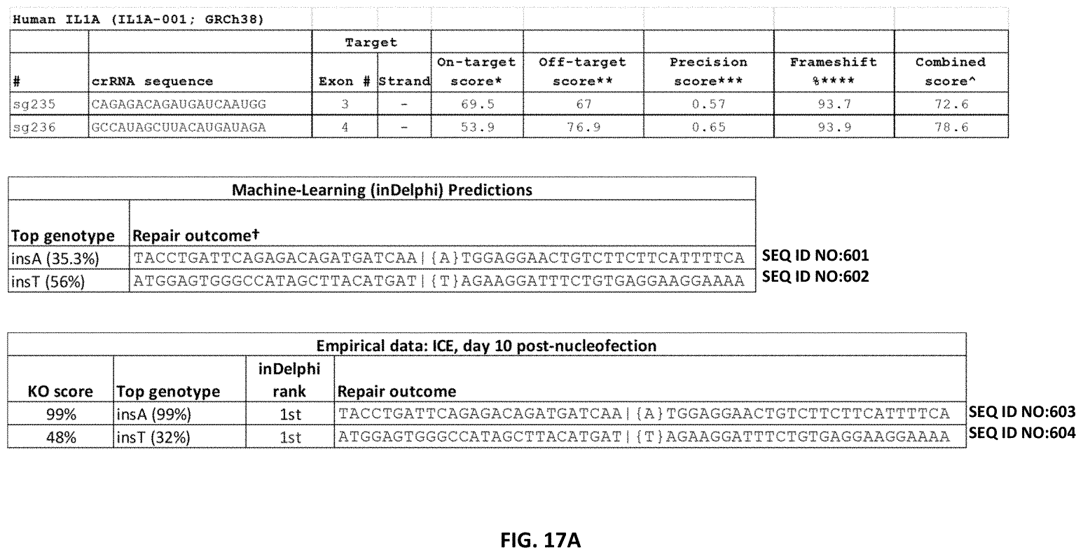

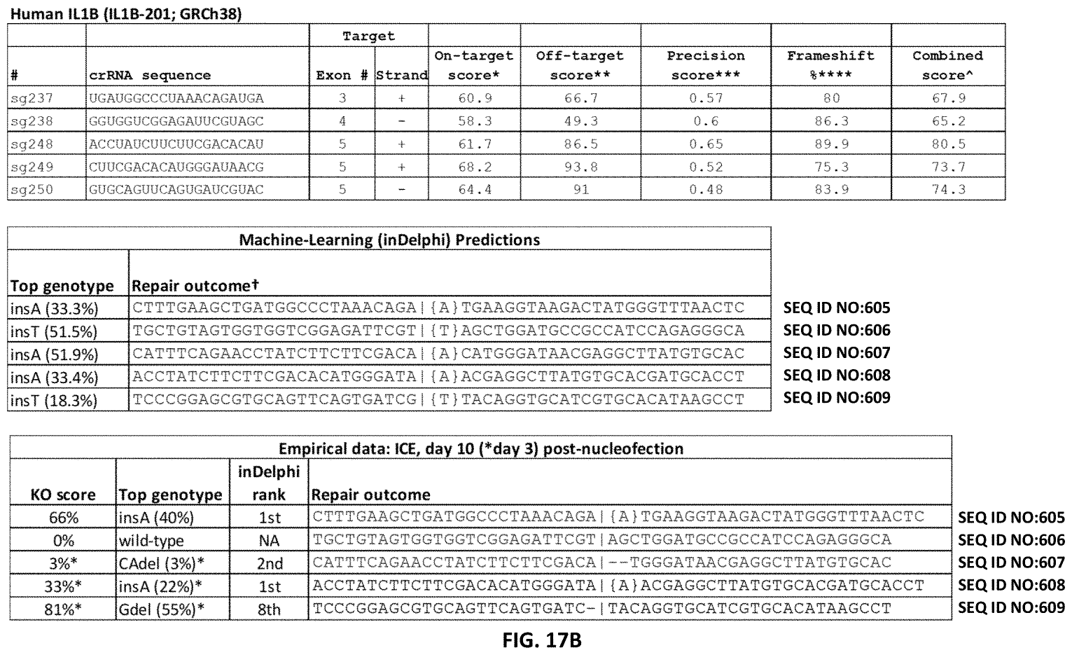

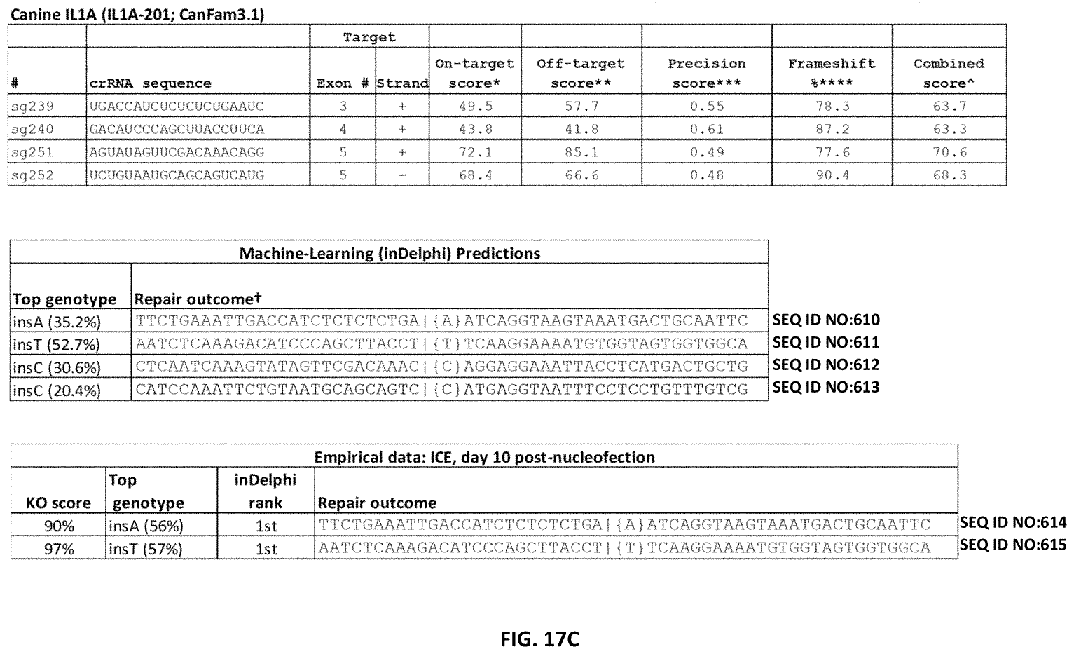

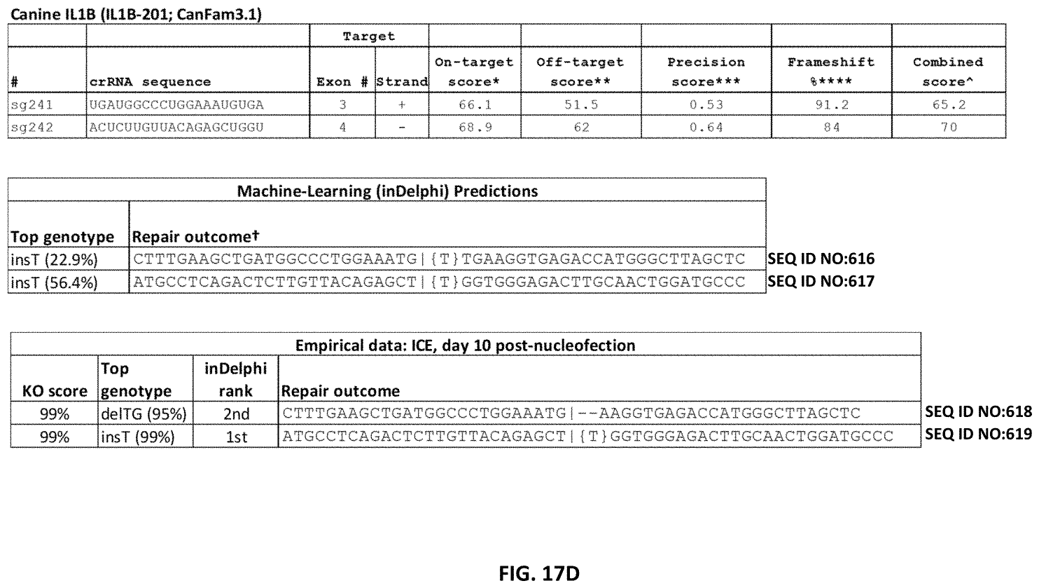

Using this selection criteria, crRNA guide sequences targeting different exons of the respective target genes were selected for further investigation. Specifically, as shown in FIG. 17A, sg235 (SEQ ID NO:301) and sg236 (SEQ ID NO:309) target exons 3 and 4 of the human IL-1α gene were selected. Likewise, as shown in FIG. 17B, sg237 (SEQ ID NO:462), sg238 (SEQ ID NO:391), sg248 (SEQ ID NO:393), sg249 (SEQ ID NO:388), and sg250 (SEQ ID NO:389) targeting exons 3, 4, and 5 of the human IL-1β gene were selected. As shown in FIG. 17C, sg239 (SEQ ID NO:552), sg240 (SEQ ID NO:554), sg251 (SEQ ID NO:578), and sg252 (SEQ ID NO:579) targeting exons 3, 4, and 5 of the canine IL-1α gene were selected. Likewise, as shown in FIG. 17D, sg241 (SEQ ID NO:498) and sg242 (SEQ ID NO:506) targeting exons 3 and 4 of the canine IL-1β gene were selected.

Single guide RNAs (sgRNAs), fusing the selected crRNA guide sequences to a scaffold sequence were then synthesised (Synthego) with scaffold modifications designed to increase their stability and decrease their cellular immunogenicity. Primers for genotyping were designed to be at least 200 bp from the target site and generate PCR amplicons <1.5 kb and synthesized (Merck).

The following quantities were used for single electroporation-based transfection using the 4D-nucleofector (Lonza, Catalog AAF-1002B and AAF-1002X) and nucleocuvette strips. 80 pmol synthesised sgRNA were pre-complexed with 4 μg Cas9 nuclease at room temperature for at least 10 min. 300-400K dissociated cells were washed with PBS before resuspending them in 20 μl supplemented P3 nucleofection solution and adding the Cas9 RNP complex. These cells were then transferred into a nucleocuvette well and electroporated using the pulse code ER-100. Directly after electroporation, the nucleocuvette was placed into the 37° C./5% CO2 incubator for 10 min for the cells to recover from the electrical voltage. Afterwards, 80 μl growth medium was added to the nucleocuvette well and cells transferred into 6-well dishes with prewarmed growth medium.

Between two- and eleven-days post-electroporation, genomic DNA was extracted from 50-200K cells using DNeasy Blood & Tissue kit (Qiagen, Catalog 69506). Single gRNA target (and off-target) regions were amplified by PCR.

PCR products were size-verified by gel electrophoresis, purified using QIAquick PCR purification kit (Qiagen, Catalog 28106) and submitted for Sanger sequencing at Source BioScience. Sanger traces (abl) were deconvoluted using ICE version 1.2 (found online at the URL github.com/synthego-open/ice) to infer CRISPR edits. In addition, machine-learning predictions of gene editing using the selected probes was generated using inDelphi. In addition, the predicted off-target sites were analysed through direct sequencing to verify whether gRNA facilitates off-target editing.

Results of the empirical experiments and machine-learning prediction of gene editing using the selected guide sequences is shown in FIG. 17.

### Example 9.—Effect of Selected CRISPR/Cas9 RNA Guides on Canine and Human Interleukin-1 Alpha (IL-1α) and Interleukin-1 Beta (IL-1β) Release

The gRNA with the highest knockout (KO) scores from Example 8 (i.e., the highest frameshift frequency) were used to generate double IL-1α/IL-1β knock out (KO) cells. Specifically, human chondrocytes were edited to achieve >99% IL-1α KO using crRNA sequence CAGAGACAGAUGAUCAAUGG (SEQ ID NO:301) and 67% IL-1β KO using crRNA sequence GUGCAGUUCAGUGAUCGUAC (SEQ ID NO:389). Canine chondrocytes were edited to achieve 97% IL-1α KO using crRNA sequence GACAUCCCAGCUUACCUUCA (SEQ ID NO:554) and 99% IL-1β KO using crRNA sequence ACUCUUGUUACAGAGCUGGU (SEQ ID NO:506).

Canine chondrocytes (Catalog Cn402K-05), human chondrocytes (Catalog 402-05a) and human fibroblast-like synovial cells (Catalog 408-05a) were purchased as frozen stocks (5×10\hat{ }5 cells) from Cell Applications, Inc., San Diego, Calif. Chondrocytes were cultured in growth medium consisting of DMEM/Ham's F12 (Gibco, Catalog 21331-020) supplemented with 20% (v/v) untreated FBS (Gibco, Catalog 10270-106) and 1× GlutaMAX (Gibco, Catalog 35050-038). Synovial cells were cultured in growth medium consisting of DMEM (Gibco, Catalog 11960-044), 10% non-treated FBS (Gibco, Catalog 10270-106) and 1× GlutaMAX (Gibco, Catalog 35050-038). Cells were confirmed as being negative for Mycoplasma spp. and subjected to STR profiling prior to use. For electroporation and subculture, cells were dissociated using 0.25% trypsin (Gibco, Catalog 25200056). Trypsin was quenched with 9 volumes of growth medium and cells were spun at 1,000 g to remove the supernatant.

Induction of IL-1 by LPS. Interleukin-1 release was induced by challenging sub-confluent monolayers of cells (edited or wild-type non-edited) with lipopolysaccharide (LPS). In brief, non-edited (control) and double IL-1α/IL-1β KO (edited) human or canine chondrocytes were seeded at density of approximately 5×10⁴ cells per well in 24-well plates. After 24-48 hours, the medium was replaced with fresh, serum-free medium containing either LPS (50 μg/ml) or PBS vehicle and the plates returned to the incubator. Plates were harvested after 6 and 24 hours for the determination of IL-1 release. Media were snap-frozen in liquid nitrogen and stored at −20° C. until they were assayed.

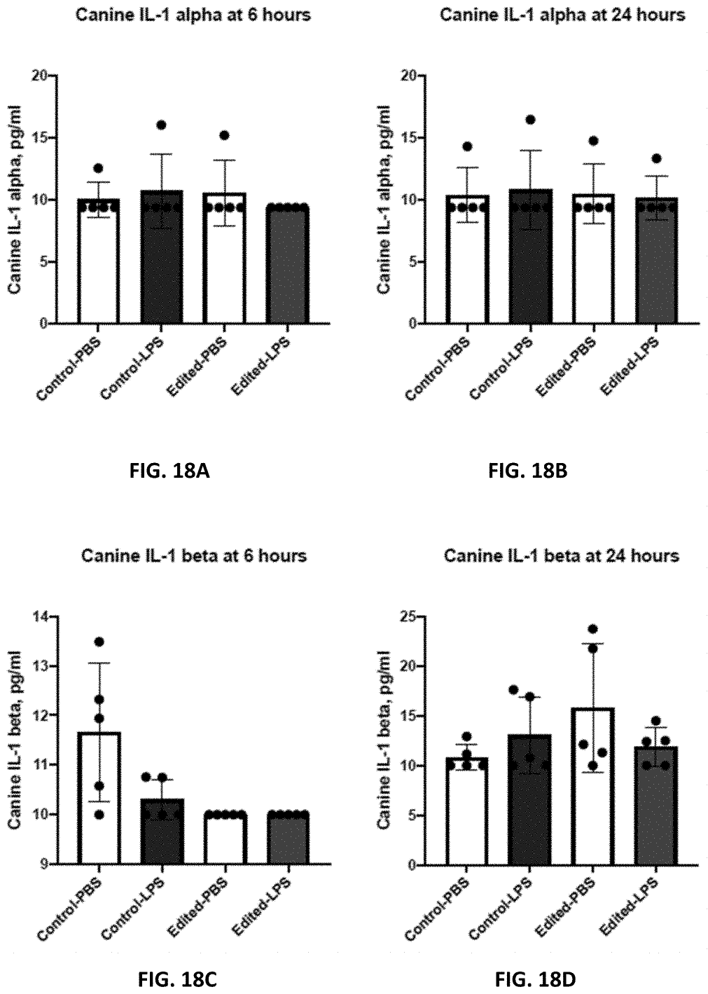

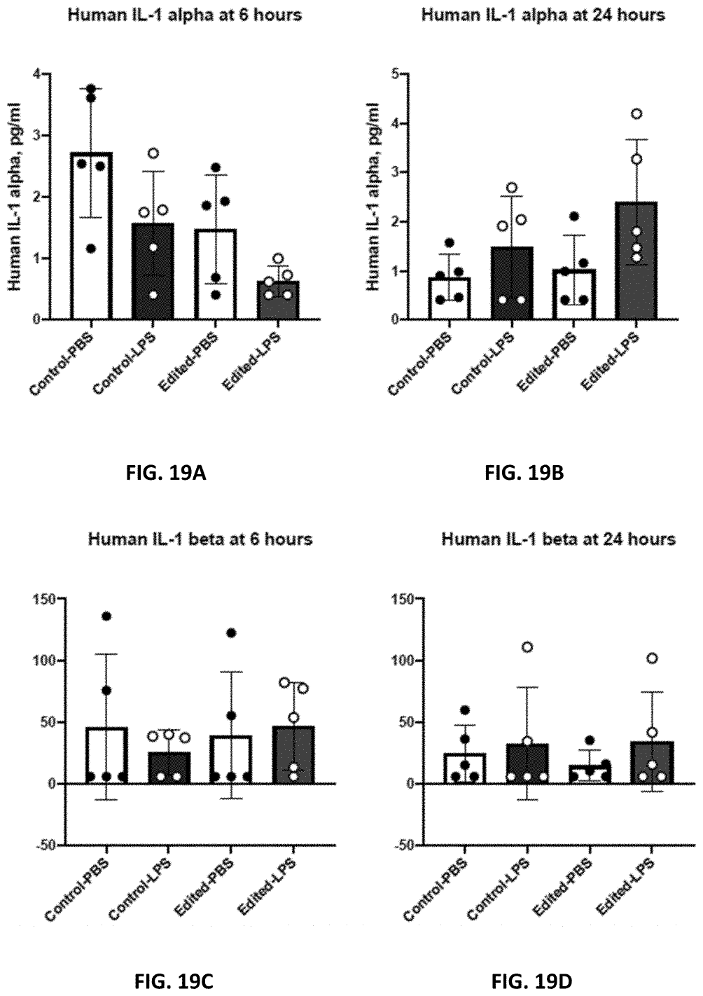

Measurement of IL-1 alpha and IL-1 beta release. The concentration of IL-1 alpha and IL-1 beta in culture medium was measured with species-specific commercial assays, following the manufacturer's instructions. Prior to measurement, frozen media were thawed and then centrifuged (1,500 g for 2 mins) in order to remove cellular debris. Aliquots of medium were measured in duplicate and the concentration of IL-1 determined from a standard curve of recombinant human or canine IL-1 alpha or beta, as appropriate. The results of IL-1 alpha release in canine cells are shown in FIGS. 18A (6 hours) and 18B (24 hours). The results of IL-1 beta release in canine cells are shown in FIGS. 18C (6 hours) and 18D (24 hours). The results of IL-1 alpha release in human cells are shown in FIGS. 19A (6 hours) and 19B (24 hours). The results of IL-1 beta release in human cells are shown in FIGS. 19C (6 hours) and 19D (24 hours).

P3 primary cell nucleofection reagents and nucleocuvette strips (Catalog V4XP-3032) were purchased from Lonza (Slough, UK). Cas9 nuclease (Catalog A36499) was purchased from Thermo Fisher Scientific. Lipopolysaccharide (LPS) from E. coli O55:B5 (Catalog L6529) was purchased from Merck. ELISA kits for human IL-1 alpha (Catalog ab214025) and human IL-1 beta (Catalog ab100560), canine IL-1 alpha (Catalog A4270) and canine IL-1 beta (Catalog ab273170) were purchased from Abcam (Cambridge, UK).

### Example 10. Increased Specificity of CRISPR/Cas9 Mediated Gene Editing

The analysis of gene editing specificity reported in Example 8 was repeated using an enhanced Specificity CRISPR associated protein 9. The eSpCas9 includes three specificity enhancing mutations: K848A, K1003A, and R1060A, as described in Slaymaker et al., Science, 351:84-88 (2016). The SpCas9 was expressed in E. coli and purified to homogeneity. The construct has a molecular weight of 161 kDa and contains N-terminal Flag-tags and a C-terminal hexa-His-tag. The sequence of the eSpCas9 is:

(SEQ ID NO: 680)
MDYKDHDGDYKDHDIDYKDDDDKMAPKKKRKVGIHGVPAADKKYSIGLD
IGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSN
FDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYY
VGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKV
MKQLKRRRYTGWGRLSRKLINGIRDKQSGKTILDFLKSDGFANRNFMQL
IHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVP
QSFLADDSIDNKVLTRSDKNRGKSDNVPSEEVVKKMKNYWRQLLNAKLI
TQRKFDNLTKAERGGLSELDKAGFIKRQLVETRQITKHVAQILDSRMNT
KYDENDKLIREVKVITLKSKLVSDFRKDFQFYKVREINNYHHAHDAYLN
AVVGTALIKKYPALESEFVYGDYKVYDVRKMIAKSEQEIGKATAKYFFY
SNIMNFFKTEITLANGEIRKAPLIETNGETGEIVWDKGRDFATVRKVLS
MPQVNIVKKTEVQTGGFSKESILPKRNSDKLIARKKDWDPKKYGGFDSP
TVAYSVLVVAKVEKGKSKKLKSVKELLGITIMERSSFEKNPIDFLEAKG
YKEVKKDLIIKLPKYSLFELENGRKRMLASAGELQKGNELALPSKYVNF
LYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDR
KRYTSTKEVLDATLIHQSITGLYETRIDLSQLGGDKRPAATKKAGQAKK
KKAAALEHHHHHH.

Briefly, the same sgRNAs used in Example 8, and shown in FIG. 17, were complexed with the eSpCas9. As shown in FIG. 17A, sg235 (SEQ ID NO:301) and sg236 (SEQ ID NO:309) target exons 3 and 4 of the human IL-1α gene were used. Likewise, as shown in FIG. 17B, sg237 (SEQ ID NO:462), sg238 (SEQ ID NO:391), sg248 (SEQ ID NO:393), sg249 (SEQ ID NO:388), and sg250 (SEQ ID NO:389) targeting exons 3, 4, and 5 of the human IL-1β gene were used. As shown in FIG. 17C, sg239 (SEQ ID NO:552), sg240 (SEQ ID NO:554), sg251 (SEQ ID NO:578), and sg252 (SEQ ID NO:579) targeting exons 3, 4, and 5 of the canine IL-1α gene were used. Likewise, as shown in FIG. 17D, sg241 (SEQ ID NO:498) and sg242 (SEQ ID NO:506) targeting exons 3 and 4 of the canine IL-1β gene were used.

Single guide RNAs (sgRNAs), fusing the selected crRNA guide sequences to a scaffold sequence were then synthesised (Synthego) with scaffold modifications designed to increase their stability and decrease their cellular immunogenicity. Primers for genotyping were designed to be at least 200 bp from the target site and generate PCR amplicons <1.5 kb and synthesized (Merck).

The following quantities were used for single electroporation-based transfection using the 4D-nucleofector (Lonza, Catalog AAF-1002B and AAF-1002X) and nucleocuvette strips. 80 pmol synthesised sgRNA were pre-complexed with eSpCas9 nuclease at room temperature for at least 10 min. 300-400K dissociated cells were washed with PBS before resuspending them in 20 μl supplemented P3 nucleofection solution and adding the Cas9 RNP complex. These cells were then transferred into a nucleocuvette well and electroporated using the pulse code ER-100. Directly after electroporation, the nucleocuvette was placed into the 37° C./5% CO2 incubator for 10 min for the cells to recover from the electrical voltage. Afterwards, 80 μl growth medium was added to the nucleocuvette well and cells transferred into 6-well dishes with prewarmed growth medium.

Between two- and eleven-days post-electroporation, genomic DNA was extracted from 50-200K cells using DNeasy Blood & Tissue kit (Qiagen, Catalog 69506). Single gRNA target (and off-target) regions were amplified by PCR.

PCR products were size-verified by gel electrophoresis, purified using QIAquick PCR purification kit (Qiagen, Catalog 28106) and submitted for Sanger sequencing at Source BioScience. Sanger traces (abl) were deconvoluted using ICE version 1.2 (found online at the URL github.com/synthego-open/ice) to infer CRISPR edits. In addition, machine-learning predictions of gene editing using the selected probes was generated using inDelphi. In addition, the predicted off-target sites were analysed through direct sequencing to verify whether gRNA facilitates off-target editing.

As compared to the Cas9 editing reported in Example 8, use of eSpCas9 reduces off-target editing without losing on-target activity. For example, the off-target editing by sgRNA #242 (targeting canine IL-1B) of three loci, having 2, 3, and 3 mismatches, respectively, were evaluated by amplifying and then sequencing the loci reported in Table 19. As shown in Table 19, the first off-target loci experienced no editing in the experiment described in Example 8, and was not tested here. The second off-target loci experienced almost complete off-target editing (98-99%) in the experiment described in Example 8, but experienced no editing when eSpCas9 was used. The third off-target loci experienced some editing (0-25%) in the experiment described in Example 8, but again experienced no editing when eSpCas9 was used. Further, as shown in Table 18, the “enhanced on-target score,” corresponding to editing using eSpCas9 as described in this example, for each sgRNA tested was as high, if not higher, than the “on-target score,” corresponding to the editing described in Example 8.

TABLE 18

| | | | On-target | | | | Off-target |
|---|---|---|---|---|---|---|---|
| | | | metrics | | | Enhanced | metrics |
| | Target | | On-target | Precision | Frameshift | on-target | Off-target |
| # | Exon # | Strand | score* | score** | %*** | score**** | score\hat{ } |
| sg235 | 3 | − | 69.5 | 0.57 | 93.7 | 72.4 | 67 |
| sg236 | 4 | − | 53.9 | 0.65 | 93.9 | 66.7 | 76.9 |

Human IL1B (IL1B-201; GRCh38)

| | | | | | | | |
|---|---|---|---|---|---|---|---|
| sg237 | 3 | + | 60.9 | 0.57 | 80 | 64.7 | 66.7 |
| sg238 | 4 | − | 58.3 | 0.6 | 86.3 | 65.7 | 49.3 |
| sg248 | 5 | + | 61.7 | 0.65 | 89.9 | 69.6 | 86.5 |
| sg249 | 5 | + | 68.2 | 0.52 | 75.3 | 65.9 | 93.8 |
| sg250 | 5 | − | 64.4 | 0.48 | 83.9 | 65.2 | 91 |

Canine IL1A (IL1A-201; CanFam3.1)

| | | | | | | | |
|---|---|---|---|---|---|---|---|
| sg239 | 3 | + | 49.5 | 0.55 | 78.3 | 58.1 | 57.7 |
| sg240 | 4 | + | 43.8 | 0.61 | 87.2 | 59.0 | 41.8 |
| sg251 | 5 | + | 72.1 | 0.49 | 77.6 | 67.7 | 85.1 |
| sg252 | 5 | − | 68.4 | 0.48 | 90.4 | 68.8 | 66.6 |

Canine IL1B (IL1B-201; CanFam3.1)

| | | | | | | | |
|---|---|---|---|---|---|---|---|
| sg241 | 3 | + | 66.1 | 0.53 | 91.2 | 69.1 | 51.5 |
| sg242 | 4 | − | 68.9 | 0.64 | 84 | 71.5 | 62 |

TABLE 19

| | | | | | | | | | CRISPR |
|---|---|---|---|---|---|---|---|---|---|
| | | | | | | | | | Edits with |
| | | | | | | | | CRISPR | enhanced |
| | | | | | | | | Edits | specificity |
| Sequence | PAM | Score | Gene | Chromosome | Strand | Position | Mismatches | with Cas9 | Cas9 |
| ACTCTTGTTACAGAGCTGGT | GGG | 100 | ENSCAFG00000007249 | chr17 | 1 | 37022194 | 0 | | |
| ACTTTTGTTTCAGAGCTGGT | CAG | 6.16161972 | | chr33 | -1 | 20234937 | 2 | 0% | |
| CCTCATGCTACAGAGCTGGT | GGG | 2.76564774 | | chr1 | -1 | 47541563 | 3 | 98-99% | 0% |
| GTGCTTGTTACAGAGCTGGT | GGG | 2.32143742 | | chr26 | -1 | 32323843 | 3 | 0-25% | 0% |

### Example 11.—Selection of gRNAs Targeting IL1a and IL1b in Human and Canine Patients

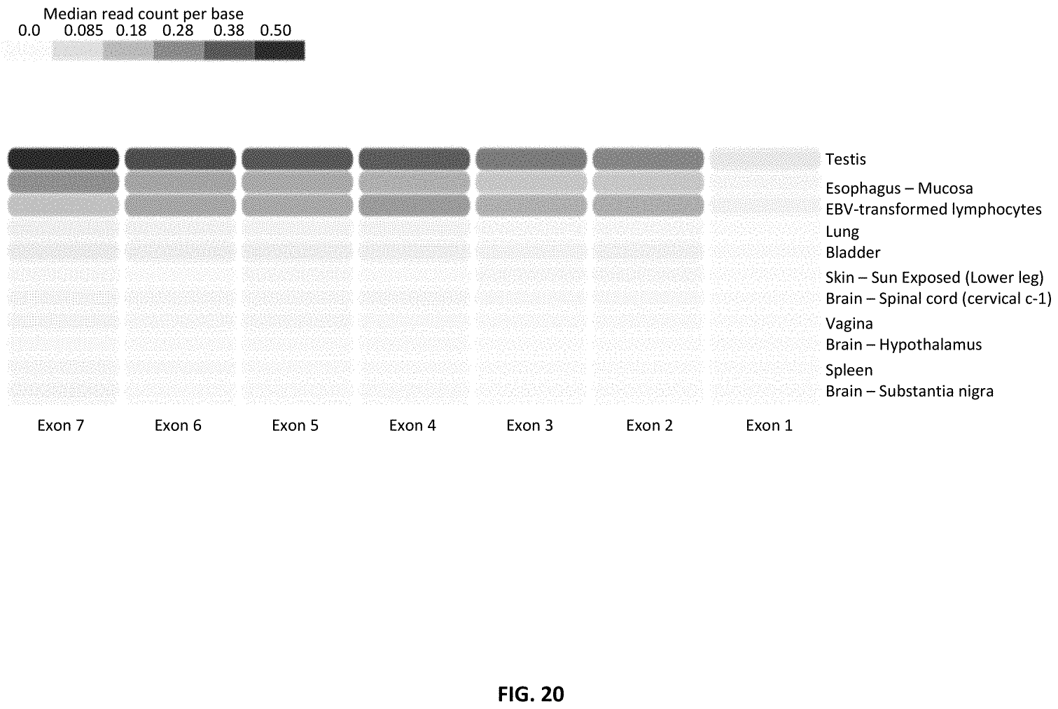

Given that the ultimate goal of CRISPR target design is fabrication of nucleotide sequences that will hybridize to genomic DNA sequences resulting in the most robust knockout of a targeted gene as part of the CRISPR/Cas system, the process begins with assessment of splicing at the target loci. Human IL-1a (hIL-1a) exhibits almost exclusively canonical splicing (i.e., no major variants) across various tissue types with the mature mRNA including exons 2-7, making each of these a potential CRISPR gRNA target (FIG. 20). Additional functional analysis (see Michlits, et al. (2020). Nature Methods, 17(7), 708-716) of the hIL-1a gene demonstrated that all functional domains cluster within Exons 5-7. In order to avoid a truncation that retains residual post-editing functionality, hIL-1a CRISPR targets were limited to those upstream of the functional domain cluster (exons 2-4). In so doing, a resultant frameshift or premature stop codon (i.e., missense mutation) at the editing site will impact all functional domains.

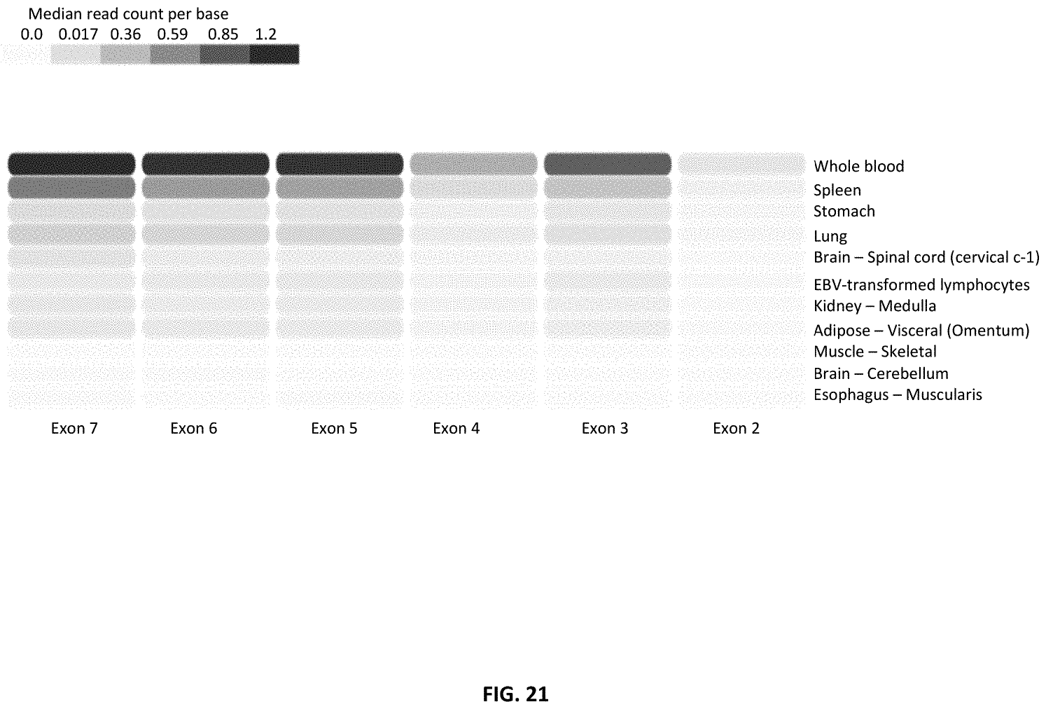

Similar analysis of human IL-1b (hIL-1b) found a stronger overall expression pattern and more variation in splicing as compared to hIL-1α (FIG. 21). However, as no tissue exhibited a variant omitting exons 2-7, each of these remained viable CRISPR targets. Application of the same functional analysis tools for hIL-1b found that functional domains cluster in exons 5-7, leaving exons 2-4 as viable CRISPR targets.

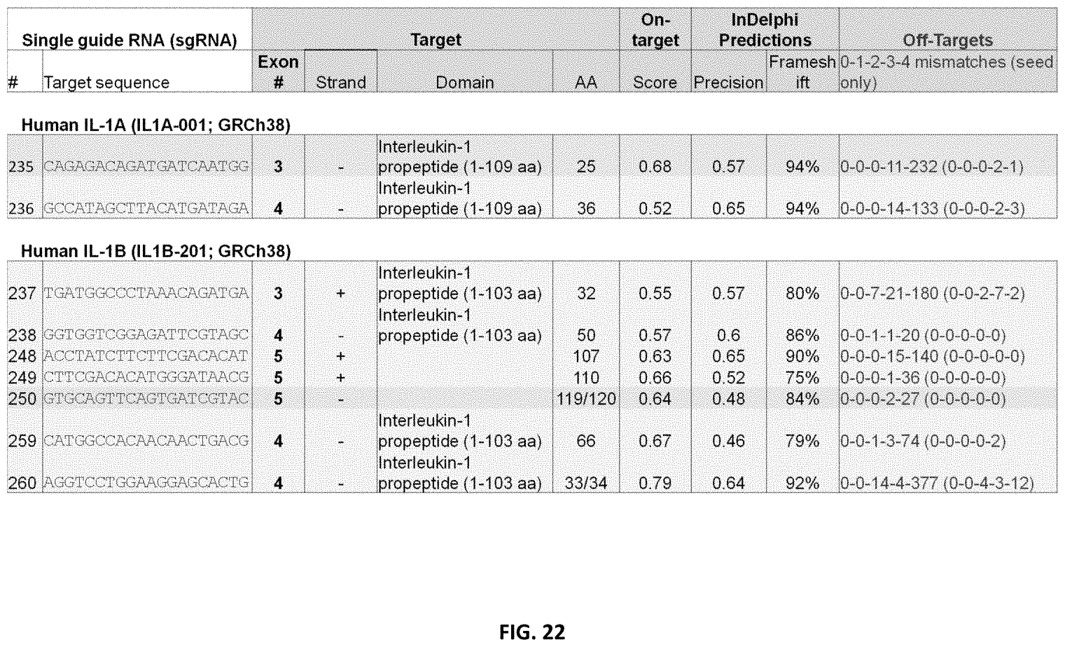

Having established the human gene targets, emphasis then shifted to gRNA targeting domain design. Generated CRISPR targeting domains were first tested in silico through at least, four separate algorithms, yielding scores assessing cutting activity (On-Target score; see Doench et al. (2016). Nature Biotechnology, 34(2), 184-191), reproducibility of the particular mutation via double-strand break repair mechanisms (Precision score; inDelphi), likelihood of creating a frameshift mutation (Frameshift score; inDelphi), and specificity of gRNA binding (Off-Target score; CRISPOR) (FIG. 22). Cutoffs were set for On-Target score at >0.30 and for Off-Target scores of 0 for 0 or 1 mismatch (first two columns).

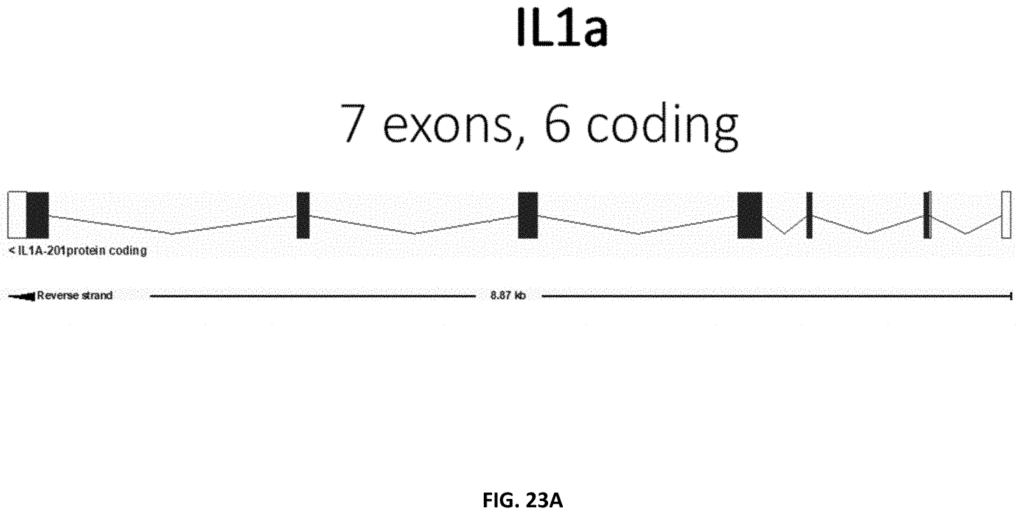

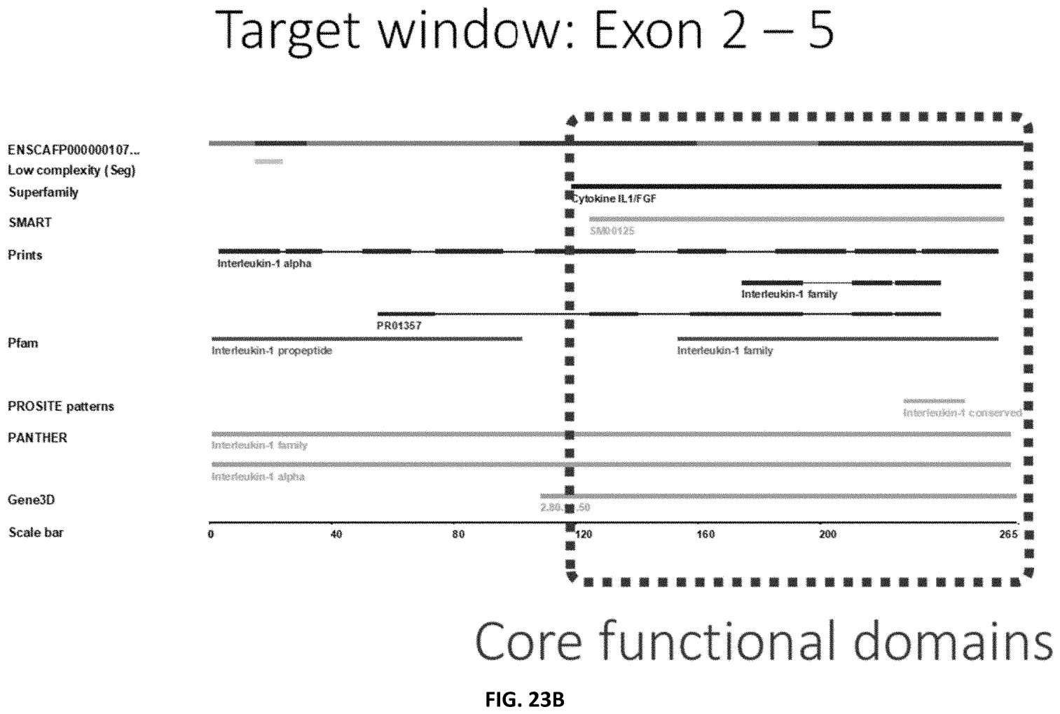

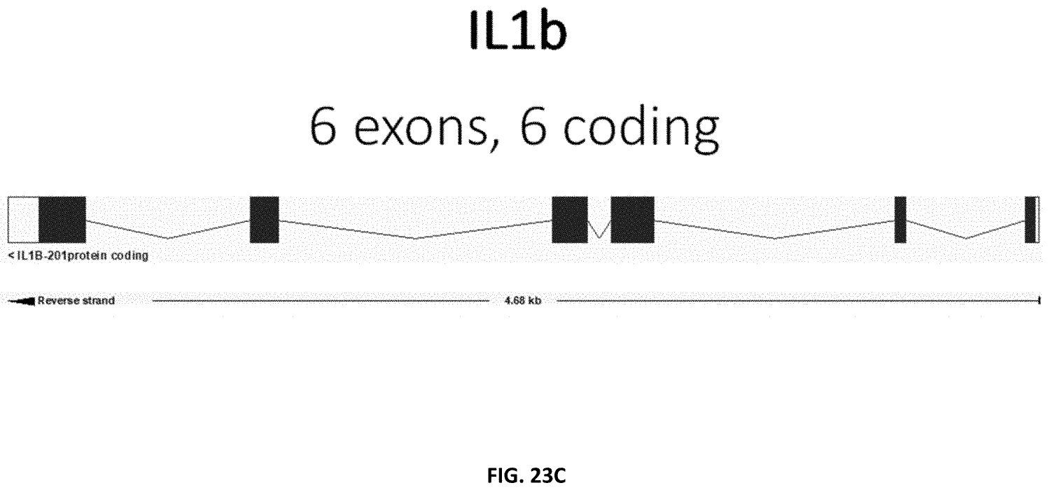

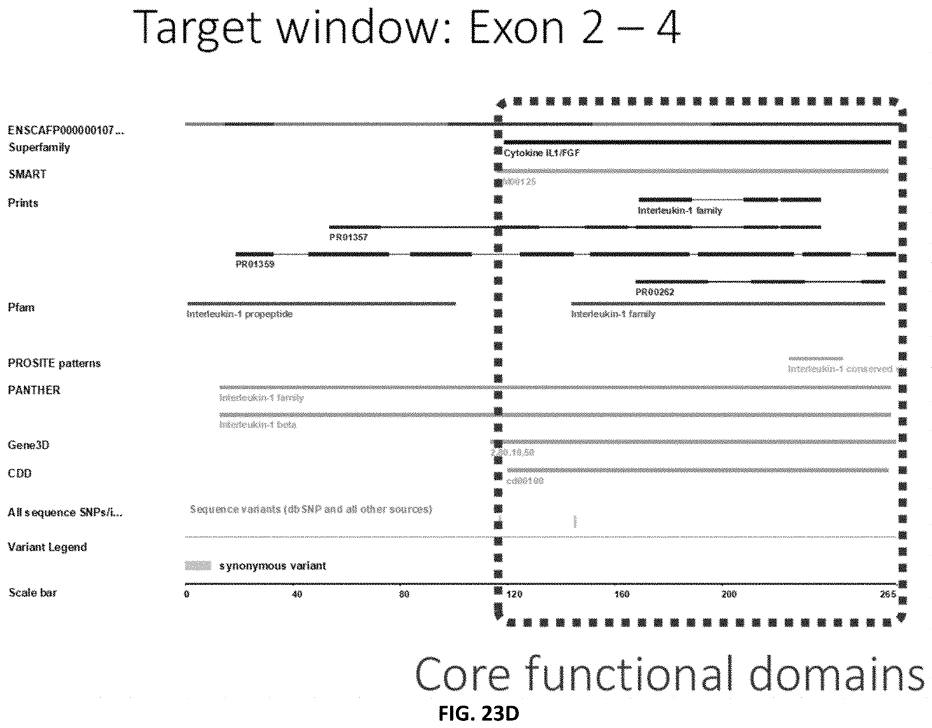

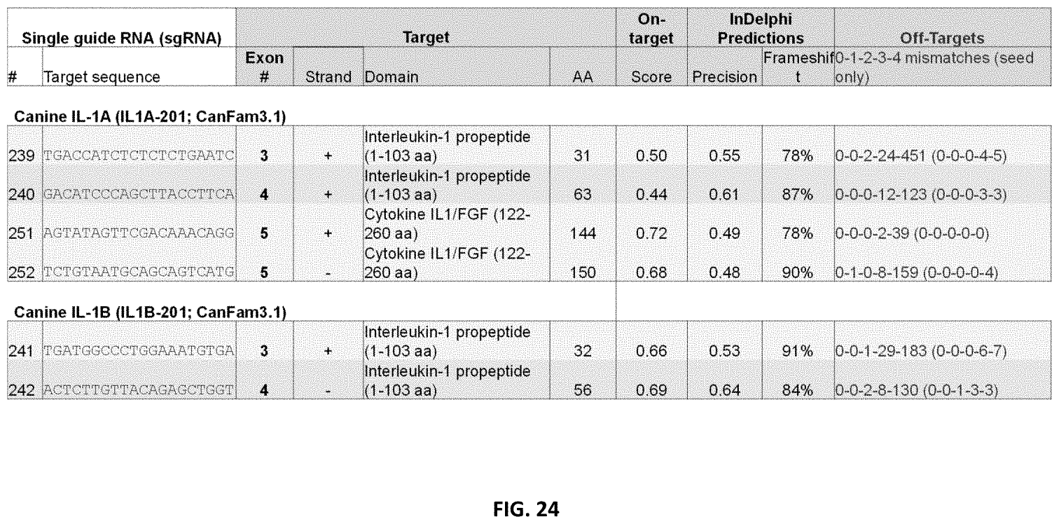

The same design process was then repeated for the orthologous canine gene targets. Splicing and functional analyses of canine IL-1a (cIL-1α) shows 6 exons (exons 2-7) incorporated in the mature mRNA, of which exons 6 and 7 contain functional domains (FIG. 23A). This leaves exons 2-5 as viable CRISPR targets. The mature mRNA of canine IL-1b (cIL-1b) contains exons 2-7, with the core function domains clustered in exons 5-7 (FIG. 23B). As such, exons 2-4 are potentially viable CRISPR targets. Generated candidate CRISPR targeting domains were then analyzed in silico; those exceeding the minimum cutoff scores are shown in FIG. 24.

### Example 12. Characterizing the Ablation of IL1a and IL1b in Primary Human and Canine Cells

Algorithm-validated gRNA targeting domains were then tested in primary cells. Briefly, plasmid DNA encoding a sgRNA with the selected targeting domain was introduced into the primary cells with an encoded Cas9 plasmid via electroporation. Pooled cell populations then underwent DNA extraction and sequencing to assess editing efficiency.

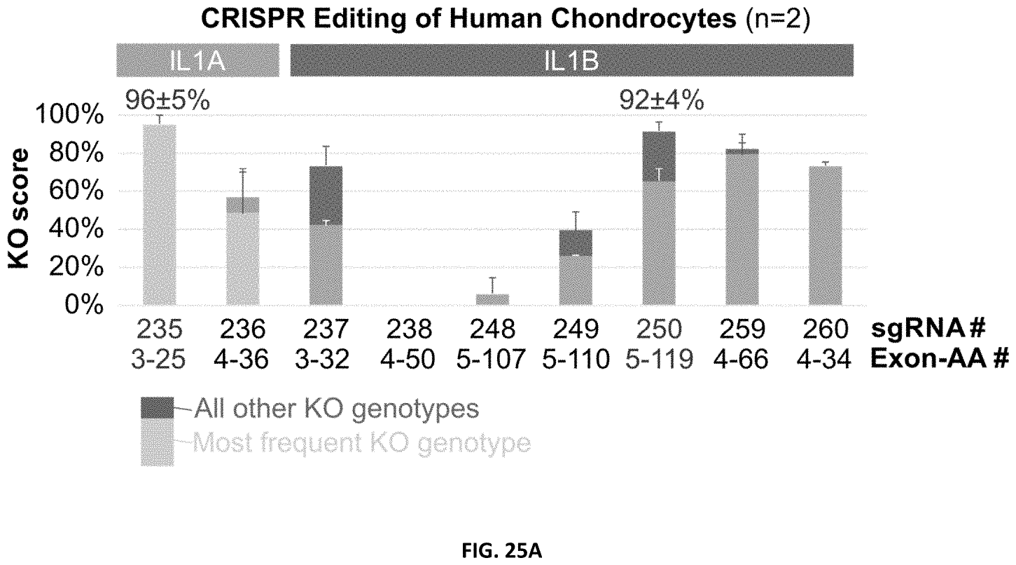

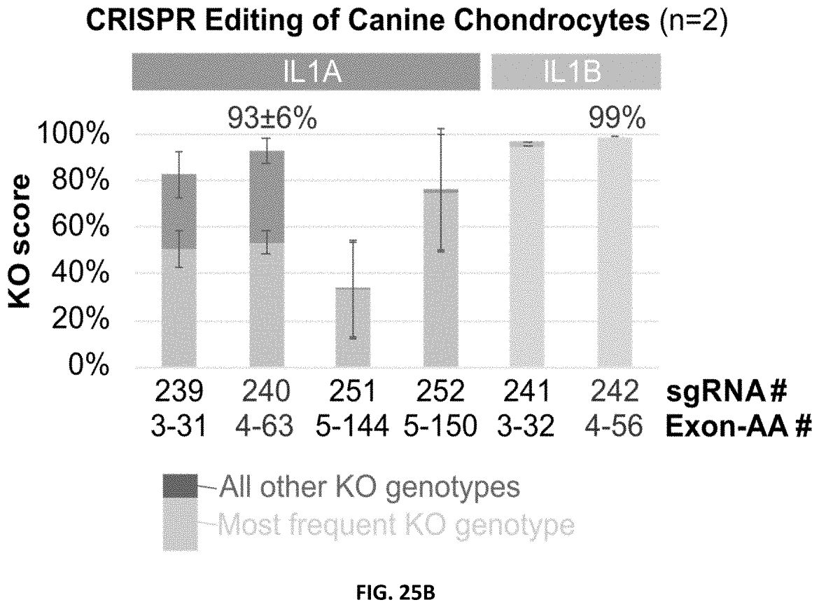

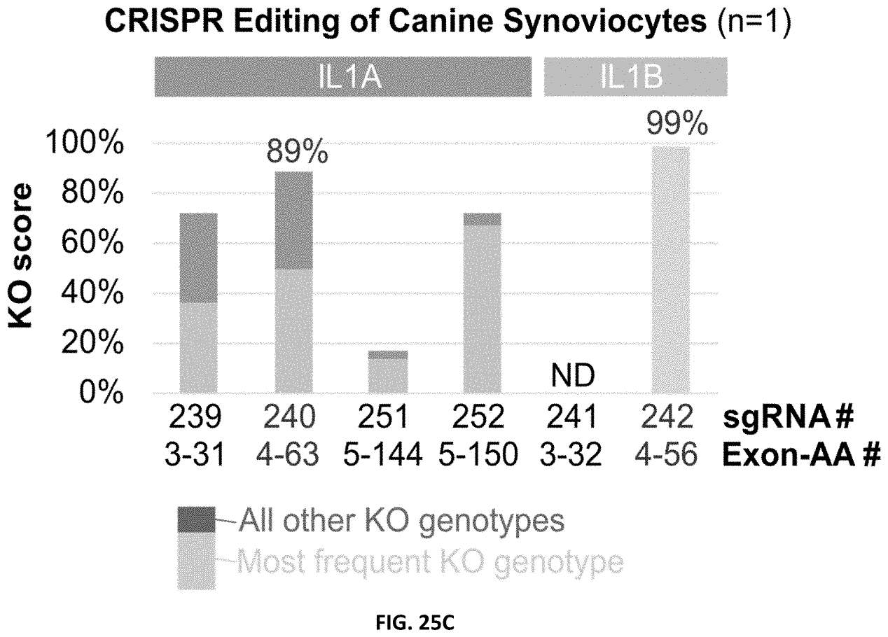

The results, shown in FIG. 25, demonstrate a wide range of editing efficiencies in both human and canine cells. Indeed, for each gene target, at least one targeting domain demonstrates effective editing (between 89% and 99%) with reproducibility between different cell types (in canine).

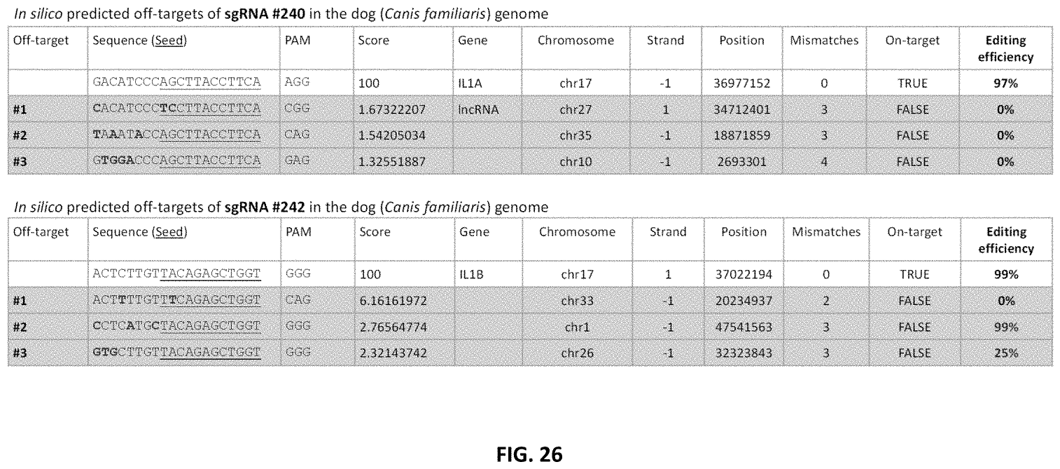

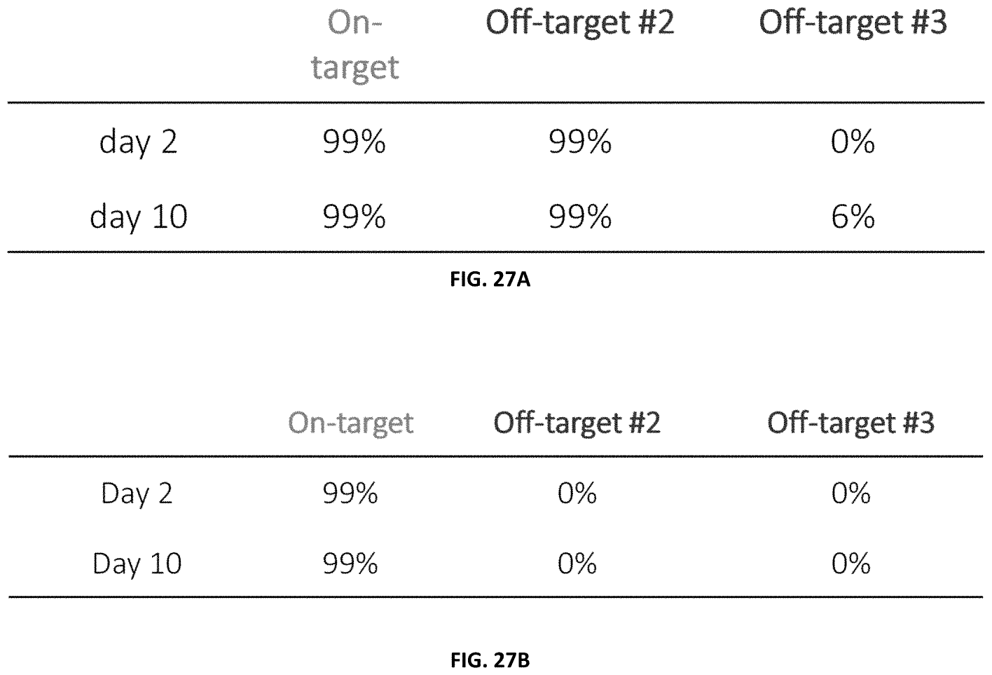

However, in addition to this robust editing of the gene target, sgRNA 242, which targets cIL-1b, also exhibited high levels of off-target editing, as anticipated by the in silico analysis (FIG. 26). To rectify these off-target effects, the experiments were repeated with an engineered, enhanced-specificity Cas9 (eSpCas9). This engineered Cas9 completely abrogated the previously-observed off-target effects while still maintain maximal editing efficiency at the target site (FIG. 27). These data demonstrate that the lead candidate sgRNA targeting domain for each gene target can safely (i.e., without off-target effects) and reliably generate genetic knockouts through creation of a primary missense mutation within the targeted locus in human (FIG. 28A) and canine (FIG. 28B) chondrocytes.

### Example 13. Co-Administration of IL1a- and IL1b-Targeted gRNAs in Primary Human and Canine Cells

Having observed robust and reproducible efficacy for each individual gRNA targeting domain, the lead candidates were next assessed in the context of co-administration in order to generate double knockouts. To do this, each sgRNA was administered to canine synoviocytes as described in Example 2 either simultaneously or sequentially (in either order). The results show that sgRNA 242 is highly effective at knocking out cIL-1b under all conditions (FIG. 29). Conversely, sgRNA 240, targeting cIL-1α, demonstrated optimal efficacy when it edits first with both simultaneous and secondary administration reducing efficacy by roughly 30%. However, such significant reduction likely remains beyond the threshold needed to impact functionality at the organismal level.

### Example 14. Extension to Additional Species

Interleukin 1 is a highly conserved gene in terms of both sequence and function among mammals (see Dinarello, C. A. (1991). Blood 77 (8): 1627-1652). As a result, the gRNA targeting domains that have been generated and characterized for specific species may result in efficient editing of the IL-1 locus of additional species. An alignment to discover conserved IL1A (FIG. 30A) and IL1B (FIG. 30B) gene target positions among humans, horses, dogs, and mice finds relatively few mismatched base pairs across all species at particular target positions. For example, sgRNA 239, targeting cIL-1α, is predicted to also edit mouse IL-1α. Given the reported flexibility within the CRISPR/Cas system to tolerate imperfect sequence alignments under certain conditions (see generally Zischewski, J., et al. (2017). Biotechnology Advances, 35(1), 95-104), cross-species reactivity for particular targeting domains is wholly anticipated.

### Example 15. Assessing Impact of Knockout on the cIL-1α Gene

Having observed CRISPR-mediated knock out of the IL-1α gene in canine chondrocytes using guides sg239, sg240, sg251, and sg252 (Example 12 and FIG. 28B), it was tested whether these guides could mediate effective and consistent IL-1A editing in a range of canine cells that are believed to mediate inflammatory effects in joint disease, namely chondrocytes, monocytes, and synoviocytes. Briefly, sgRNA was precomplexed with wild-type SpCas9 and electroporated into primary canine chondrocytes (experimental n=3), monocytes (experimental n=2), and synoviocytes (experimental n=3) with an encoded Cas9 plasmid via electroporation. Pooled cell populations then underwent DNA extraction and sequencing to assess editing efficiency.

As shown in FIG. 31A, efficient editing was achieved across all three cell types when using sg252 (average efficiency=81%), sg240 (average efficiency=90%), and sg239 (average efficiency=79%). In contrast, use of sg251 resulted in an average editing efficiency of only 25% across all three cell types, despite having a higher predicted on-target editing score than each of sg239, sg240, and sg252 (as shown in Table 18). Next, the homogeneity of observed editing was evaluated. As shown in FIG. 31A, editing with sg252 resulted in nearly homogenous incorporation of a single guanine nucleotide (96% of edits). In contrast, editing with sg239 and sg240 was less homogenous. Specifically, 54% of all edits using sg240 resulted in incorporation of a thymidine nucleotide, while 58% of all edits using sg239 resulted in incorporation of an adenine nucleotide.

Each of the most common edits caused by sg239, sg240, sg251, and sg252 is a single nucleotide addition. As shown in FIG. 31B, these single nucleotide additions cause a frameshift at amino acid position 61 that introduces a premature stop codon at amino acid position 91. Protein folding modeling using AlphaFold2 was performed to evaluate whether the truncated protein caused by the editing would retain any IL-1A structure. FIG. 31C shows the predicted structure for the wild-type IL-1A (top) and truncated polypeptide (bottom). As can be seen, the truncated protein is unstructured, suggesting that it does not retain any biological functions of the wild-type IL-1A. Taken together, these results suggest that editing of the IL-1α gene disrupts all IL-1α activity.

Finally, the most efficient guide (sgRNA240) was used to generate two double IL1A/B KO lines of canine monocytes DH82 achieving at least 96% IL1A and 99% IL1B knockout scores.

### Example 16. Comparative Analyses of Guide RNAs Targeting the cIL-1α Gene

To identify additional sgRNA targeting IL-1A with low off-target editing effects, four additional putative target sequences were identified based on in silico prediction of off-target score. FIG. 32 shows the target sequence for four previously generated anti-IL-1A sgRNAs: sg239 (renamed as OCA01), sg240 (renamed as OCA03), sg251 (renamed as OCA07), and sg252 (renamed as OCA08), as well as the target sequence for four newly generated sgRNA having low predicted off-target effects: sg358 (renamed as OCA05), sg359 (renamed as OCA04), sg360 (renamed as OCA02), and sg361 (renamed as OCA06). Briefly, putative target sequences were evaluated by multiple models for each of on-target editing efficiency, off-target editing effects, and frameshift edits. Scores for each putative target sequence are shown in FIG. 32B.

Off-target editing effects were predicted by averaging scores generated by the MIT model and the CFD model. The MIT algorithm, also known as Hsu-Zhang score. Hsu, P. D. et al. DNA targeting specificity of RNA-guided Cas9 nucleases. Nature Biotechnology 31, 827-832 (2013). This model is based on a positional penalty matrix (1×20) generated from 15 EMX1 sgRNA libraries with mismatches against target at every position. The CFD algorithm (Cutting Frequency Determination) is based on threat matrix (12×20) considering both position and mismatch type and PAM integrity (27,897 ‘CD33’ sgRNAs+10,618 negative control sgRNAs). Doench, J. G. et al. Optimized sgRNA design to maximize activity and minimize off-target effects of CRISPR-Cas9. Nature Biotechnology 34, 184-191 (2016).

On-target editing efficiencies were predicted by averaging scores generated by the Azimuth model, the DeepSpCas9 model, and the CrisprScan model. The Azimuth model is a boosted regression tree model, trained with 881 sgRNAs (MOLM13/NB4/TF1 cells+unpublished additional data) delivered by lentivirus. Doench, J. G. et al. Optimized sgRNA design to maximize activity and minimize off-target effects of CRISPR-Cas9. Nature Biotechnology 34, 184-191 (2016). DeepSpCas9 is a deep learning model trained using editing data from 12,832 sgRNA. Kim, H. K. et al. SpCas9 activity prediction by DeepSpCas9, a deep learning-based model with high generalization performance. Science Advances 5, (2019). CrisprScan is a linear regression model, trained using editing data from 1000 sgRNAs injected into zebrafish embryos targeting >100 genes. Moreno-Mateos, M. A. et al. CRISPRscan: designing highly efficient sgRNAs for CRISPR-Cas9 targeting in vivo. Nature Methods 12, 982-988 (2015).

The putative guides' potential to generate frameshift mutations were predicted by averaging scores generated by the Lindel model and the InDelphi model. Lindel is a machine learning model trained using profile data of 1.16 million independent mutational events triggered by CRISPR/Cas9-mediated cleavage and non-homologous end joining-mediated double strand break repair of 6872 synthetic target sequences, introduced into a human cell line via lentiviral infection. Chen, W. et al. Massively parallel profiling and predictive modeling of the outcomes of CRISPR/Cas9-mediated double-strand break repair. Nucleic Acids Research 47, 7989-8003 (2019). InDelphi is machine learning model trained with indels generated by 1872 sgRNAs. Shen, M. W. et al. Predictable and precise template-free CRISPR editing of pathogenic variants. Nature 563, 646-651 (2018).

The editing efficiency of these guides was then tested in canine monocytes DH82. Briefly, sgRNA was precomplexed with wild-type SpCas9 and electroporated into primary canine monocytes DH82. Pooled cell populations then underwent DNA extraction and sequencing to assess editing efficiency. As shown in FIG. 32C, OCA01-OCA05 and OCA08 mediated efficient knock-out editing. Further, OCA05 and OCA08 did so with consistent single nucleotide addition edits.

### Example 17. Comparative Analyses of Guide RNAs Targeting the cIL-1β Gene

To identify additional sgRNA targeting IL-1B with low off-target editing effects, four additional putative target sequences were identified based on in silico prediction of off-target score. FIG. 33A shows the target sequence for two previously generated anti-IL-1B sgRNAs: sg241 (renamed as OCB01) and sg242 (renamed as OCB02), as well as the target sequence for six newly generated sgRNA having low predicted off-target effects: sg352 (renamed OCB06), sg353 (renamed as OCB04), sg354 (renamed as OCB08), sg355 (renamed as OCB05), sg356 (renamed as OCB07), and sg357 (renamed as OCB03). Briefly, putative target sequences were evaluated by multiple models for each of on-target editing efficiency, off-target editing effects, and frameshift edits, as described in Example 16. Scores for each putative target sequence are shown in FIG. 33B.

The editing efficiency of these guides was then tested in canine monocytes DH82. Briefly, sgRNA was precomplexed with wild-type SpCas9 and electroporated into primary canine monocytes DH82. Pooled cell populations then underwent DNA extraction and sequencing to assess editing efficiency. As shown in FIG. 33C, OCB01-02 and OCB04-06 mediated efficient knock-out editing. Further, OCB01, OCB02, and OCB04 did so with consistent edits. The low editing efficiencies of sgRNAs OCB3 (61%) and OCB07 (0%) were predicted in silico by low on-target scores (FIG. 33B; 39 and 32 points, respectively). However, the poor performance of OCB08 could not be explained by the in silico metrics.

Finally, these genetic effects were further confirmed by performing anti-IL-1B ELISA assays on the supernatant of edited cells. As shown in FIG. 33D, each of the sgRNA that showed efficient editing by sequence analysis also resulted in significant reduction in IL-1B secretion by cells targeted by the guides. Taken together, these results confirm editing in canine monocytes by the designed guides.

### Example 18. Interrogation of Guide RNAs Targeting the cIL-1β Gene

Top sgRNA candidates were further assessed for their impact on the cIL-1β gene and protein. First, in a repeat of the in vitro sequencing assay, extremely consistent results are observed (FIG. 34A). Indeed, a single edit occurs in the edited cells in over 90% of cells, suggesting strong reproducibility when taken together with previous results. Next, both cIL-1-targeting candidates were assessed in the context of a double knockout with an sgRNA targeting cIL-1a. Not only all sgRNAs retain strong knockout efficiency (FIG. 34B, left panel), but these edits result in disruption of protein function. Supernatant ELISA results show that secretion of cIL-1β protein is significantly abrogated, with reductions of more than 95% compared to control. Taken together, these results suggest that the lead sgRNA candidates retain potency, even when combined.

### Example 19. Assessment of cIL-1β-Targeting sgRNA in Primary Monocytes

Monocytes are among the most important cells in cellular immunity (and autoimmunity), given their ability to differentiate into numerous different cell fates. As such, these cells are among the prime targets of therapies directed to joint diseases. Therefore, understanding the impact of gene editing on these particular cells becomes pivotal.

Several sgRNA candidates targeting cIL-1β were introduced to canine monocytes via electroporation. The edited cells were then subsequently challenged with LPS for either 6 or 24 hours, at which time supernatants were analyzed via ELISA. Results show that negligible amount of IL-1β are detected in LPS-challenged monocytes that had been edited with either OCB01 or OCB02 (FIGS. 35A, B). Conversely, while control cells respond robustly at both time points.

The same assay was then repeated for a broader panel of sgRNAs. Again, strong inhibition of IL-1β secretion at 24 hours is observed across the board (FIG. 36). In no instance was average inhibition less than 80% of the unedited control.

### Example 20. Impact of Mutant Cas9 Proteins with Enhanced Specificity

It was then investigated whether OCB02 caused off-targeting editing by sequencing two genomic loci in the canine monocytes edited as described in Example 17 with high sequence identity to the target loci (Off-target #2 and Off-target #3). As shown in FIG. 38, when complexed with wild-type Cas9, OCB02 caused high levels of off targeting editing at both loci.

To determine whether this off-target editing could be avoided by using Cas9 variants known to have enhanced specificity, canine monocytes were edited with either OCB01 or OCB02 precomplexed with wild-type Cas9, ES-Cas9 (Enhanced Specificity Cas9, described in Slaymaker, I. M. et al. Rationally engineered Cas9 nucleases with improved specificity. Science (1979) 351, (2016)), PE-Cas9 (PURedit® Cas9; Merck), HF-Cas9 (TrueCut® HiFi Cas9; Invitrogen), or AR-Cas9 (Alt-R® HiFi Cas9; IDT). The on-target editing efficiency of these complexes was evaluated by sequencing the IL1B gene in the edited cells. As shown in FIG. 38, all of the high efficiency Cas9 variants mediated efficient on-targeting editing (IL1B knockout) when paired with the OCB02 sgRNA. However, only some of the high fidelity Cas9 variants mediated efficient on-targeting editing (IL1B knockout) when paired with the OCB01 sgRNA. The effect of the high fidelity Cas9 proteins on off-targeting editing with the OCB02 sgRNA were then evaluated by sequencing off-target sites #2 and #3 in edited cells. As shown in FIG. 38, each of the high fidelity Cas9 proteins eliminated off-target editing at these loci.

The effect of the high-fidelity Cas9 variants on IL-1B expression/secretion was then tested, as described above. As expected from the sequencing results, editing using each of the enhanced specificity Cas9 proteins resulted in efficient inhibition of IL1B secretion 24 hours following LPS challenge (FIG. 37A). Similar results were shown for OCB01-mediated editing using AR-Cas9, as expected from the sequencing data (FIG. 37B).

The examples set forth above are provided to give those of ordinary skill in the art a complete disclosure and description of how to make and use the embodiments of the compositions, systems and methods of the disclosure, and are not intended to limit the scope of what the inventors regard as their invention. Modifications of the above-described modes for carrying out the embodiments of the disclosure that are obvious to persons of skill in the art are intended to be within the scope of the following claims. All patents and publications mentioned in the specification are indicative of the levels of skill of those skilled in the art to which the disclosure pertains.

### Example 21. Identification of Example sgRNA Targeting Human, Canine, Equine, or Feline IL-1A or IL-1β Genes

To identify additional sgRNA targeting IL-1A or IL-1B in silico predictions of on-target editing efficiency, off-target editing effects, and frameshift editing probability were generated for putative target human, canine, equine, and feline sequences as described in Example 16, except that for human guide sequences the on-target scores that were averaged also included a score generated using the Elevation model. Elevation is an end-to-end machine learning model trained by GUIDE-Seq and other aggregated data. See, Listgarten, J. et al. Prediction of off-target activities for the end-to-end design of CRISPR guide RNAs. Nature Biomedical Engineering 2, 38-47 (2018); and Tsai, S. Q. et al. GUIDE-seq enables genome-wide profiling of off-target cleavage by CRISPR-Cas nucleases. Nature Biotechnology 33, 187-197 (2015). An overall score for each putative crRNA sequence was then calculated as the average of the on-target, off-target, and frameshift score. The highest scoring crRNA sequences for each gene and species were selected. Scores for each putative target sequence are shown in FIGS. 39A-46B.

The genomic location of the exons reported in FIGS. 39A-46B is provided in Tables 20-27, below.

TABLE 20
Human IL1A

| | | | UCSC | | | | |
|---|---|---|---|---|---|---|---|
| Exon | | | Genome | | | | |
| # | Coding | Ensembl ID | Assembly ID | Chromosome | Strand | Start | End |
| 1 | No | ENSE00001325622 | hg38 | chr2 | reverse | 112,784,493 | 112,784,443 |
| 2 | Yes | ENSE00001128612 | hg38 | chr2 | reverse | 112,783,778 | 112,783,724 |
| 3 | Yes | ENSE00001145252 | hg38 | chr2 | reverse | 112,782,764 | 112,782,716 |
| 4 | Yes | ENSE00000856832 | hg38 | chr2 | reverse | 112,781,826 | 112,781,604 |
| 5 | Yes | ENSE00000773893 | hg38 | chr2 | reverse | 112,779,666 | 112,779,496 |
| 6 | Yes | ENSE00000773892 | hg38 | chr2 | reverse | 112,778,111 | 112,777,987 |
| 7 | Yes | ENSE00001128606 | hg38 | chr2 | reverse | 112,775,267 | 112,773,925 |

TABLE 21
Human IL1B

| | | | UCSC | | | | |
|---|---|---|---|---|---|---|---|
| Exon | | | Genome | | | | |
| # | Coding | Ensembl ID | Assembly ID | Chromosome | Strand | Start | End |
| 1 | No | ENSE00001000625 | hg38 | chr2 | reverse | 112,836,779 | 112,836,708 |
| 2 | Yes | ENSE00003508205 | hg38 | chr2 | reverse | 112,836,244 | 112,836,183 |
| 3 | Yes | ENSE00003647827 | hg38 | chr2 | reverse | 112,835,617 | 112,835,566 |
| 4 | Yes | ENSE00003506405 | hg38 | chr2 | reverse | 112,833,575 | 112,833,374 |
| 5 | Yes | ENSE00003582426 | hg38 | chr2 | reverse | 112,832,826 | 112,832,662 |
| 6 | Yes | ENSE00003615183 | hg38 | chr2 | reverse | 112,831,422 | 112,831,292 |
| 7 | Yes | ENSE00000856835 | hg38 | chr2 | reverse | 112,830,573 | 112,829,751 |

TABLE 22
Canine IL1A

| | | | UCSC | | | | |
|---|---|---|---|---|---|---|---|
| Exon | | | Genome | | | | |
| # | Coding | Ensembl ID | Assembly ID | Chromosome | Strand | Start | End |
| 1 | No | ENSCAFE00000309751 | canFam3 | chr17 | reverse | 36,979,435 | 36,979,356 |
| 2 | Yes | ENSCAFE00000078619 | canFam3 | chr17 | reverse | 36,978,720 | 36,978,666 |
| 3 | Yes | ENSCAFE00000078621 | canFam3 | chr17 | reverse | 36,977,683 | 36,977,635 |
| 4 | Yes | ENSCAFE00000078623 | canFam3 | chr17 | reverse | 36,977,242 | 36,977,032 |
| 5 | Yes | ENSCAFE00000078624 | canFam3 | chr17 | reverse | 36,975,266 | 36,975,093 |
| 6 | Yes | ENSCAFE00000078626 | canFam3 | chr17 | reverse | 36,973,248 | 36,973,130 |
| 7 | Yes | ENSCAFE00000078627 | canFam3 | chr17 | reverse | 36,970,929 | 36,970,573 |

TABLE 23
Canine IL1B

| | | | UCSC | | | | |
|---|---|---|---|---|---|---|---|
| Exon | | | Genome | | | | |
| # | Coding | Ensembl ID | Assembly ID | Chromosome | Strand | Start | End |
| 1 | No | NA | canFam3 | chr17 | reverse | NA | NA |
| 2 | Yes | ENSCAFE00000409713 | canFam3 | chr17 | reverse | 37,024,176 | 37,023,944 |
| 3 | Yes | ENSCAFE00000241094 | canFam3 | chr17 | reverse | 37,023,405 | 37,023,354 |
| 4 | Yes | ENSCAFE00000078700 | canFam3 | chr17 | reverse | 37,022,260 | 37,022,065 |
| 5 | Yes | ENSCAFE00000078702 | canFam3 | chr17 | reverse | 37,021,958 | 37,021,797 |
| 6 | Yes | ENSCAFE00000241077 | canFam3 | chr17 | reverse | 37,020,557 | 37,020,427 |
| 7 | Yes | ENSCAFE00000309007 | canFam3 | chr17 | reverse | 37,019,681 | 37,019,329 |

TABLE 24
Equine IL1A

| | | | UCSC | | | | |
|---|---|---|---|---|---|---|---|
| Exon | | | Genome | | | | |
| # | Coding | Ensembl ID | Assembly ID | Chromosome | Strand | Start | End |
| 1 | No | ENSECAE00000193601 | equCab3 | chr15 | reverse | 16,369,812 | 16,369,762 |
| 2 | Yes | ENSECAE00000193613 | equCab3 | chr15 | reverse | 16,369,119 | 16,369,065 |
| 3 | Yes | ENSECAE00000193626 | equCab3 | chr15 | reverse | 16,368,070 | 16,368,022 |
| 4 | Yes | ENSECAE00000193646 | equCab3 | chr15 | reverse | 16,367,110 | 16,366,888 |
| 5 | Yes | ENSECAE00000193669 | equCab3 | chr15 | reverse | 16,365,310 | 16,365,137 |
| 6 | Yes | ENSECAE00000193696 | equCab3 | chr15 | reverse | 16,364,119 | 16,363,998 |
| 7 | Yes | ENSECAE00000193723 | equCab3 | chr15 | reverse | 16,361,752 | 16,360,700 |

TABLE 25
Equine IL1B

| | | | UCSC | | | | |
|---|---|---|---|---|---|---|---|
| Exon | | | Genome | | | | |
| # | Coding | Ensembl ID | Assembly ID | Chromosome | Strand | Start | End |
| 1 | No | ENSECAE00000001011 | equCab3 | chr15 | reverse | 16,444,697 | 16,444,653 |
| 2 | Yes | ENSECAE00000000363 | equCab3 | chr15 | reverse | 16,423,448 | 16,423,390 |
| 3 | Yes | ENSECAE00000269159 | equCab3 | chr15 | reverse | 16,422,859 | 16,422,808 |
| 4 | Yes | ENSECAE00000000420 | equCab3 | chr15 | reverse | 16,422,550 | 16,422,346 |
| 5 | Yes | ENSECAE00000000449 | equCab3 | chr15 | reverse | 16,421,794 | 16,421,633 |
| 6 | Yes | ENSECAE00000000487 | equCab3 | chr15 | reverse | 16,420,363 | 16,420,233 |
| 7 | Yes | ENSECAE00000308144 | equCab3 | chr15 | reverse | 16,419,536 | 16,418,738 |

TABLE 26
Feline IL1A

| | | | UCSC | | | | |
|---|---|---|---|---|---|---|---|
| Exon | | | Genome | | | | |
| # | Coding | Ensembl ID | Assembly ID | Chromosome | Strand | Start | End |
| 1 | No | NA | felCat9 | chrA3 | forward | NA | NA |
| 2 | Yes | ENSFCAE00000082073 | felCat9 | chrA3 | forward | 105,113,193 | 105,113,247 |
| 3 | Yes | ENSFCAE00000473731 | felCat9 | chrA3 | forward | 105,114,215 | 105,114,263 |
| 4 | Yes | ENSFCAE00000345284 | felCat9 | chrA3 | forward | 105,115,053 | 105,115,275 |
| 5 | Yes | ENSFCAE00000082076 | felCat9 | chrA3 | forward | 105,117,059 | 105,117,232 |
| 6 | Yes | ENSFCAE00000082077 | felCat9 | chrA3 | forward | 105,118,594 | 105,118,715 |
| 7 | Yes | ENSFCAE00000293842 | felCat9 | chrA3 | forward | 105,120,921 | 105,121,209 |

TABLE 27
Feline IL1B

| | | | UCSC | | | | |
|---|---|---|---|---|---|---|---|
| Exon | | | Genome | | | | |
| # | Coding | Ensembl ID | Assembly ID | Chromosome | Strand | Start | End |
| 1 | Yes? | ENSFCAE00000421394 | felCat9 | chrA3 | forward | 105,049,334 | 105,050,213 |
| 2 | Yes | ENSFCAE00000060114 | felCat9 | chrA3 | forward | 105,050,633 | 105,050,694 |
| 3 | Yes | ENSFCAE00000060119 | felCat9 | chrA3 | forward | 105,051,232 | 105,051,280 |
| 4 | Yes | ENSFCAE00000060121 | felCat9 | chrA3 | forward | 105,052,908 | 105,053,112 |
| 5 | Yes | ENSFCAE00000060125 | felCat9 | chrA3 | forward | 105,053,655 | 105,053,816 |
| 6 | Yes | ENSFCAE00000060131 | felCat9 | chrA3 | forward | 105,055,063 | 105,055,193 |
| 7 | Yes | ENSFCAE00000398371 | felCat9 | chrA3 | forward | 105,056,165 | 105,056,407 |

All headings and section designations are used for clarity and reference purposes only and are not to be considered limiting in any way. For example, those of skill in the art will appreciate the usefulness of combining various aspects from different headings and sections as appropriate according to the spirit and scope of the disclosure described herein.

It is to be understood that the methods described herein are not limited to the particular methodology, protocols, subjects, and sequencing techniques described herein and as such can vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

While some embodiments have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure.

Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein can be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

1. A composition for the treatment or prevention of a joint disease or condition, comprising:
(i) an RNA-guided nuclease or a nucleic acid encoding an RNA-guided nuclease; and
(ii) at least one guide RNA or a nucleic acid encoding at least one guide RNA targeting an IL-1α gene or an IL-1β gene, wherein the guide RNA specifically binds a target sequence that is adjacent to a protospacer adjacent motif (PAM) sequence for the RNA-guided nuclease.

2. (canceled)

3. The composition of claim 1, wherein the at least one guide targets exon 2, exon 3, exon 4, exon 5, or exon 6 of the IL-1α gene or the IL-1β gene.

4. The composition of claim 3, wherein the at least one guide RNA targets a human IL-1α gene and comprises a crRNA sequence having a sequence selected from the group consisting of SEQ ID NOs: 168-187, 298-387, and 681-710.

5-6. (canceled)

7. The composition of claim 1, wherein the at least one guide RNA targets a human IL-1β gene and comprises a crRNA sequence selected from the group consisting of SEQ ID NOs: 188-201, 388-496, and 711-740.

8-9. (canceled)

10. The composition of claim 1, wherein the at least one guide RNA targets a canine IL-1α gene and comprises a crRNA sequence selected from the group consisting of SEQ ID NOs: 202-216, 552-590, and 741-770.

11-12. (canceled)

13. The composition of claim 1, wherein the at least one guide RNA targets a canine IL-1β gene and comprises a crRNA sequence selected from the group consisting of SEQ ID NOs: 217-235, 497-551, and 771-800.

14-15. (canceled)

16. The composition of claim 1, wherein the at least one guide RNA targets an equine IL-1α gene and comprises a crRNA sequence selected from the group consisting of SEQ ID NOs: 236-255 and 801-830.

17-18. (canceled)

19. The composition of claim 1, wherein the at least one guide RNA targets an equine IL-1β gene and comprises a crRNA sequence selected from the group consisting of SEQ ID NOs: 256-262 and 831-860.

20-21. (canceled)

22. The pharmaceutical composition of claim 1, wherein the at least one guide RNA targets a feline IL-1α gene and comprises a crRNA sequence selected from the group consisting of SEQ ID NOs: 861-890.

23-24. (canceled)

25. The composition of claim 1, wherein the at least one guide RNA targets a feline IL-1β gene and comprises a crRNA sequence from the group consisting of SEQ ID NOs: 891-920.

26. The composition of claim 1, wherein the RNA-guided nuclease is a CRISPR associated protein 9 (Cas9) protein.

27. The composition of claim 26, wherein the Cas9 protein is an S. pyogenes or S. aureus Cas9 protein.

28. (canceled)

29. The composition of claim 26, wherein the Cas9 protein is an enhanced specificity Cas9 variant protein.

30. The composition of claim 1, wherein the RNA-guided nuclease or a nucleic acid encoding an RNA-guided nuclease is the RNA-guided nuclease.

31. (canceled)

32. The composition of claim 1, wherein the RNA-guided nuclease or a nucleic acid encoding an RNA-guided nuclease is mRNA encoding the RNA-guided nuclease.

33-35. (canceled)

36. The composition of claim 1, wherein the at least one guide RNA is a single guide RNA (sgRNA).

37. The composition of claim 1, wherein the composition comprises one or more lipid nanoparticles (LNP) collectively encapsulating:
(i) the RNA-guided nuclease or the nucleic acid encoding the RNA-guided nuclease; and
(ii) the at least one guide RNA or the nucleic acid encoding the at least one guide RNA.

38-51. (canceled)

52. The composition of claim 1, wherein the composition is formulated for parenteral administration.

53. The composition of claim 1, wherein the composition is formulated for intra-articular injection within a joint of a subject.

54. A method for treating a joint disease or condition, the method comprising:
administering, to a subject in need thereof, a composition comprising:
(i) an RNA-guided nuclease or a nucleic acid encoding an RNA-guided nuclease; and
(ii) at least one guide RNA or a nucleic acid encoding at least one guide RNA targeting an IL-1α or IL-1β gene, wherein the guide RNA specifically binds a target sequence that is adjacent to a protospacer adjacent motif (PAM) sequence for the RNA-guided nuclease.

55-74. (canceled)
